(19) United States
(12) Patent Application Publication
(10) Pub. No.: US 2007/0178451 A1
(43) Pub. Date: Aug. 2, 2007

(54) Nucleic acid sequences from Chlorella sarokiniana and uses thereof

(76) Inventors: Molian Deng, Grover, MO (US); Robin L. Staub, Wildwood, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 09/920,953

(22) Filed: Aug. 2, 2001

(51) Int. Cl. C12Q 1/68; C07H 21/04; C12N 1/12; C07K 14/405

(52) U.S. Cl. 435/6; 435/257.2; 530/350; 536/23.7

(57) ABSTRACT

Expressed Sequence Tags (ESTs) isolated from the unicellular green algae, Chlorella sarokiniana, are disclosed. The invention encompasses nucleic acid molecules that encode Chlorella protein homologs and fragments thereof. In addition, antibodies capable of binding the proteins are encompassed by the present invention. The disclosed ESTs have particular utility in isolating genes and promoters, identifying and mapping the genes involved in developmental and metabolic pathways, and determining gene function. The ESTs provide a unique molecular tool for the targeting and isolation of novel genes for plant protection and improvement. The invention also relates to methods of using the disclosed nucleic acid molecules, proteins, fragments of proteins, and antibodies, for example, for gene identification and analysis, and preparation of constructs.

### FIELD OF THE INVENTION

The present invention is in the field of molecular biology; more particularly, the present invention relates to nucleic acid sequences from the unicellular green algae, Chlorella sarokiniana. The invention encompasses nucleic acid molecules that encode proteins and fragments of proteins. In addition, proteins and fragments of proteins so encoded and antibodies capable of binding the proteins are encompassed by the present invention. The invention also relates to methods of using the disclosed nucleic acid molecules, proteins, fragments of proteins, and antibodies, for example, for gene identification and analysis, and preparation of constructs.

### INCORPORATION OF SEQUENCE LISTING

This application contains a sequence listing, which is contained on three identical CD-ROMs: two copies of a sequence listing (Copy 1 and Copy 2) and a sequence listing Computer Readable Form (CRF), all of which are herein incorporated by reference. All three CD-ROMs each contain one file called “pa_{—}00361.rpt” which is 6,949,411 bytes in size and was created on Jul. 26, 2001.

### BACKGROUND OF THE INVENTION

The present invention relates in part to DNA sequences from cDNA libraries from the unicellular green algae, Chlorella sorokiniana. The green algal genus Chlorella includes a variety of species (Fott and Novakova, In: Studies in Phycology: A Monograph of the Genus Chlorella, Fott, B. (ed.), Prag: Verlag Acad. Sissensch., pp. 10-74 (1969), herein incorporated by reference in its entirety), some of which have long been served as model organisms in plant physiological and biochemical studies (Govindjee and Braun, In: Algal Physiology and Biochemistry, W. D. P. Stewart (ed.), University of California Press, Berkeley and Los Angeles, pp. 346-390, herein incorporated by reference in its entirety). Chlorella belongs to the eucaryotic cell category of algae and lives in fresh water as a single cell plant. Its size is approximately 2-8 microns in diameter. Species of Chlorella have been classified by cell wall sugar composition (Takeda, Phytochemistry 27: 3823-6 (1988)) as well as other physiological and biochemical characters (Kessler, Plant Syst. Evol. 125:129-38 (1976)). The name Chlorella derives from two Latin words meaning ‘leaf’ (green) and ‘small’, referring to the unusually high content of chlorophyll which gives Chlorella its characteristic deep emerald-green color. Chlorella is also rich in protein, vitamins, minerals, “C.G.F.” (Chlorella Growth Factor) and other beneficial substances. Unicellular green algae Chlorella are currently being used to produce compounds of commercial value (Behrens et al., J. Applied Phycology 6: 113-122 (1994); Running et al., J. Applied Phycology 6: 99-104 (1994), both of which are herein incorporated by reference in their entirety).

It is generally believed that land plants evolved from green algae (Graham, J. Plant Res. 109: 241-251 (1996), herein incorporated by reference in its entirety) and that during this revolution, extensive rearrangements occurred within the chloroplast genome. The complete nucleotide sequence of the chloroplast genome (150613 bp) from the unicellular green alga Chlorella vulgaris, a species related to Chlorella sorokiniana, has been determined (Wakasugi, et al., Proc. Natl. Acad. Sci. USA 94:5967-5972 (1997), herein incorporated by reference in its entirety). The chloroplast genome of Chlorella vulgaris contains one copy of rRNA gene consisting of 16S, 23S, and 5S rRNA genes; thirty one tRNA gene, sixty-nine protein genes; eight ORFs conserved with those found in land chloroplasts; two adjacent genes homologous to bacterial genes (minD and mine) involved in cell division; genes encoding ribosomal proteins L5, L12, L19 and S9; and two long ORF's related to ycf1 and ycf2 that are exclusively found in land plants (Wakasugi, et al., Proc. Natl. Acad. Sci. USA 94: 5967-5972 (1997), herein incorporated by reference in its entirety). Chlorella is closer to land plants than the red and brown algae.

It is advantageous to identify and/or isolate chlorella genes for plant genetic engineering to produce plants with argonomically important characteristics or traits. A cDNA (or complementary DNA) library, which is constructed from mRNA purified from Chlorella cell culture, can be one valuable source for isolating genes of interest. Construction of cDNA libraries is well-known in the art and a number of cloning strategies exist. Random clones from a cDNA library can be sequenced from both 3′ and 5′ ends to generate expressed sequence tags (ESTs), which can represent copies of up to the full length transcript (McCombie, et al., Nature Genetics, 1:124-130 (1992); Kurata, et al., Nature Genetics, 8: 365-372 (1994); Okubo, et al., Nature Genetics, 2: 173-179 (1992)). Typically, only single run sequence data is obtained from the cDNA library (Adams, et al., Science 252:1651-1656 (1991)). Automated single run sequencing typically results in an approximately 2-3% error or base ambiguity rate. (Boguski, et al., Nature Genetics, 4:332-333 (1993)). Between 150-450 nucleotides of sequence information is usually generated as this is the length of sequence information that is routinely and reliably produced using single run sequence data.

ESTs have been found to be useful for similarity searches and mapping (Adams, et al., Science 252:1651-1656 (1991)). Sequence comparisons and similarity analysis would allow the identification of genes of interest and then full-length cDNA constructs can be obtained using several methods (Land, et al., Nucleic Acids Res. 9:2251-2266 (1981); Okayama and Berg, Mol. Cell Biol. 2:161 -170 (1982); Coleclough, et al., Gene 34:305-314 (1985); Krawinkel, et al., Nucleic Acids Res. 14:1913 (1986); Han, et al., Nucleic Acids Res. 15:6304 (1987)). Such isolated genes of interest can be used in plant genetic engineering to engineering to produce plants with argonomically important characteristics or traits.

### SUMMARY OF THE INVENTION

The present invention provides a substantially purified nucleic acid molecule having a nucleic acid sequence selected from the group consisting of SEQ ID NO: 1 through SEQ ID NO: 9395 or complements thereof.

The present invention also provides a substantially purified nucleic acid molecule, the nucleic acid molecule capable of specifically hybridizing to a second nucleic acid molecule having a nucleic acid sequence selected from the group consisting of SEQ ID NO: 1 through SEQ ID NO: 9395 or complements thereof.

The present invention further provides a substantially purified protein, peptide, or fragment thereof encoded by a nucleic acid sequence selected from the group consisting of SEQ ID NO: 1 through SEQ ID NO:9395 or complements thereof.

The present invention also provides a substantially purified nucleic acid molecule encoding an Chlorella sarokiniana protein homologue or fragment thereof, wherein the nucleic acid molecules comprises a nucleic acid sequence selected from the group consisting of SEQ ID NO: 1 through SEQ ID NO: 9395.

The present invention also provides a transformed cell having a nucleic acid molecule which comprises: (A) an exogenous promoter region which functions in the cell to cause the production of a mRNA molecule; which is operably linked to (B) a structural nucleic acid molecule, wherein the structural nucleic acid molecule comprises a nucleic acid sequence selected from the group consisting of SEQ ID NO: 1 through SEQ ID NO:9395 or complements thereof; which is operably linked to (C) a 3′ non-translated sequence that functions in the cell to cause termination of transcription and addition of polyadenylated ribonucleotides to a 3′ end of the mRNA molecule.

The present invention also provides a plant cell, a mammalian cell, a bacterial cell, an insect cell, a fungal cell and an algal cell transformed with a nucleic acid molecule of the present invention.

The present invention also provides a computer readable medium having recorded thereon one or more of the nucleotide sequences depicted in SEQ ID NO: 1 through SEQ ID NO: 9395 or complements thereof.

### DETAILED DESCRIPTION OF THE INVENTION

Agents of the Present Invention:

(a) Nucleic Acid Molecules

Agents of the present invention include substantially purified (or isolated) nucleic acid molecules and more specifically EST nucleic acid molecules or nucleic acid fragment molecules thereof. Fragment EST nucleic acid molecules may encode significant portion(s) of, or indeed most of, the EST nucleic acid molecule. Alternatively, the fragments may comprise smaller oligonucleotides (having from about 15 to about 250 nucleotide residues, and more preferably, about 15 to about 30 nucleotide residues).

In a preferred embodiment the nucleic acid molecules of the present invention are derived from a unicellular green alga and in an even more preferred embodiment the nucleic acid molecules of the present invention are derived from unicellular green algae belonging to the genus Chlorella. In a particularly preferred embodiment the nucleic acid molecules of the present invention are derived from Chlorella sarokiniana.

The term “nucleic acid molecule” or “nucleic acid” refers to a single or double-stranded polymer of deoxyribonucleotide or ribonucleotide bases read from the 5′ to the 3′ end. Nucleic acid molecules may also optionally contain synthetic, non-natural or altered nucleotide bases that permit correct read through by a polymerase and do not alter expression of a polypeptide encoded by that nucleic acid molecule.

As used herein “a substantially purified nucleic acid” or “an isolated nucleic acid” refers to a nucleic acid that is no longer accompanied by some of materials with which it is associated in its natural state or to a nucleic acid the structure of which is not identical to that of any of naturally occurring nucleic acid. Examples of a substantially purified nucleic acid include: (1) DNAs which have the sequence of part of a naturally occurring genomic DNA molecules but are not flanked by two coding sequences that flank that part of the molecule in the genome of the organism in which it naturally occurs; (2) a nucleic acid incorporated into a vector or into the genomic DNA of a prokaryote or eukaryote in a manner such that the resulting molecule is not identical to any naturally occurring vector or genomic DNA; (3) a separate molecule such as a cDNA, a genomic fragment, a fragment produced by polymerase chain reaction (PCR), or a restriction fragment; (4) recombinant DNAs; and (5) synthetic DNAs. A substantially purified nucleic acid may also be comprised of one or more segments of cDNA, genomic DNA or synthetic DNA.

It is also contemplated by the inventors that the substantially purified (or isolated) nucleic acids of the present invention also include known types of modifications, for example, labels which are known in the art, methylation, “caps”, substitution of one or more of the naturally occurring nucleotides with an analog. Other known modifications include internucleotide modifications, for example, those with uncharged linkages (methyl phosphonates, phosphotriesters, phosphoamidates, carbamates, etc.) and with charged linkages (phosphorothioates, phosphorodithioates, etc.), those containing pendant moieties, such as, proteins (including nucleases, toxins, antibodies, signal peptides, poly-L-lysine, etc.), those with intercalators (acridine, psoralen, etc.), those containing chelators (metals, radioactive metals, boron, oxidative metals, etc.), those containing alkylators, and those with modified linkages.

It is understood that the agents of the present invention may be labeled with reagents that facilitate detection of the agent (e.g. fluorescent labels (Prober, et al., Science 238:336-340 (1987), Albarella et al., EP 144914), chemical labels (Sheldon et a., U.S. Pat. No. 4,582,789; Albarella et al., U.S. Pat. No. 4,563,417), modified bases (Miyoshi et al., EP 119448).

The term “nucleotide sequence” or “nucleic acid sequence” refers to both the sense and antisense strands of a nucleic acid as either individual single strands or in the duplex. It includes, but is not limited to, self-replicating plasmids, chromosomal sequences, and infectious polymers of DNA or RNA.

A “coding sequence”, “structural nucleotide sequence” or “structural nucleic acid molecule” is a nucleotide sequence which is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, and recombinant nucleotide sequences.

The term “recombinant DNAs” refers to DNAs that contains a genetically engineered modification through manipulation via mutagenesis, restriction enzymes, and the like.

The term “synthetic DNAs” refers to DNAs assembled from oligonucleotide building blocks that are chemically synthesized using procedures known to those skilled in the art. These building blocks are ligated and annealed to form DNA segments which are then enzymatically assembled to construct the entire DNA. “Chemically synthesized”, as related to a sequence of DNA, means that the component nucleotides were assembled in vitro. Manual chemical synthesis of DNA may be accomplished using well established procedures, or automated chemical synthesis can be performed using one of a number of commercially available machines.

The agents of the present invention will preferably be “biologically active” with respect to either a structural attribute, such as the capacity of a nucleic acid to hybridize to another nucleic acid molecule, or the ability of a protein to be bound by antibody (or to compete with another molecule for such binding). Alternatively, such an attribute may be catalytic, and thus involve the capacity of the agent to mediate a chemical reaction or response.

It is further understood, that the present invention provides bacterial, viral, microbial, and plant cells comprising the agents of the present invention.

EST nucleic acid molecules or fragment EST nucleic acid molecules are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook, et al., In: Molecular Cloning, A Laboratory Manual, 2nd Edition, Cold Spring Harbor Press, Cold Spring Harbor, N.Y. (1989), and by Haymes, et al. In: Nucleic Acid Hybridization, A Practical Approach, IRL Press, Washington, D.C. (1985), herein incorporated by reference in its entirety. Departures from complete complementarity are therefore permissible, as long as such departures do not completely preclude the capacity of the molecules to form a double-stranded structure. Thus, in order for an EST nucleic acid molecule or fragment EST nucleic acid molecule to serve as a primer or probe it need only be sufficiently complementary in sequence to be able to form a stable double-stranded structure under the particular solvent and salt concentrations employed.

Appropriate stringency conditions which promote DNA hybridization are, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6, herein incorporated by reference in its entirety. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

In a preferred embodiment, a nucleic acid of the present invention will specifically hybridize to one or more of the nucleic acid molecules set forth in SEQ ID NO: 1 through SEQ ID NO: 9395 or complements thereof under moderately stringent conditions, for example at about 2.0×SSC and about 65° C.

In a particularly preferred embodiment, a nucleic acid of the present invention will include those nucleic acid molecules that specifically hybridize to one or more of the nucleic acid molecules set forth in SEQ ID NO: 1 through SEQ ID NO: 9395 or complements thereof under high stringency conditions.

In one aspect of the present invention, the nucleic acid molecules of the present invention have one or more of the nucleic acid sequences set forth in SEQ ID NO: 1 through to SEQ ID NO:9395 or complements thereof. In another aspect of the present invention, one or more of the nucleic acid molecules of the present invention share between 100% and 90% sequence identity with one or more of the nucleic acid sequences set forth in SEQ ID NO: 1 through to SEQ ID NO:9395 or complements thereof. In a further aspect of the present invention, one or more of the nucleic acid molecules of the present invention share between 100% and 95% sequence identity with one or more of the nucleic acid sequences set forth in SEQ ID NO: 1 through to SEQ ID NO:9395 or complements thereof. In a more preferred aspect of the present invention, one or more of the nucleic acid molecules of the present invention share between 100% and 98% sequence identity with one or more of the nucleic acid sequences set forth in SEQ ID NO: 1 through to SEQ ID NO:9395 or complements thereof. In an even more preferred aspect of the present invention, one or more of the nucleic acid molecules of the present invention share between 100% and 99% sequence identity with one or more of the sequences set forth in SEQ ID NO: 1 through to SEQ ID NO:9395 or complements thereof. In a further, even more preferred aspect of the present invention, one or more of the nucleic acid molecules of the present invention exhibit 100% sequence identity with one or more nucleic acid molecules present within the cDNA library LIB3602, herein designated (Monsanto Company, St. Louis, Mo., United States of America).

“Percentage of sequence identity” is determined by comparing two optimally aligned sequences over a comparison window, wherein the portion of the nucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Polypeptides which are “substantially similar” share sequences as noted above except that residue positions which are not identical may differ by conservative amino acid changes. Conservative amino acid substitutions refer to the interchangeability of residues having similar side chains. “Conservative amino acid substitutions” refer to substitutions of one or more amino acids in a native amino acid sequence with another amino acid(s) having similar side chains, resulting in a silent change. Conserved substitutes for an amino acid within a native amino acid sequence can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine, valine-isoleucine, phenylalanine-tyrosine, lysine-arginine, alanine-valine, aspartic acid-glutamic acid, and asparagine-glutamine.

Optimal alignment of sequences for comparison can use any means to analyze sequence identity (homology) known in the art, e.g., by the progressive alignment method of termed “PILEUP” (Morrison, Mol. Biol. Evol. 14:428-441 (1997), as an example of the use of PILEUP); by the local homology algorithm of Smith & Waterman (Adv. Appl. Math. 2: 482 (1981)); by the homology alignment algorithm of Needleman & Wunsch (J. Mol. Biol. 48:443 (1970)); by the search for similarity method of Pearson (Proc. Natl. Acad. Sci. USA 85: 2444 (1988)); by computerized implementations of these algorithms (e.g., GAP, BESTFIT, FASTA, and TFASTA in the Wisconsin Genetics Software Package, Genetics Computer Group, 575 Science Dr., Madison, Wis.); ClustalW (CLUSTAL in the PC/Gene program by Intelligenetics, Mountain View, Calif., described by, e.g., Higgins, Gene 73: 237-244 (1988); Corpet, Nucleic Acids Res. 16:10881-10890 (1988); Huang, Computer Applications in the Biosciences 8:155-165 (1992); and Pearson, Methods in Mol. Biol. 24:307-331 (1994); Pfam (Sonnhammer, Nucleic Acids Res. 26:322-325 (1998); TreeAlign (Hein, Methods Mol. Biol. 25:349-364 (1994); MEG-ALIGN, and SAM sequence alignment computer programs; or, by manual visual inspection.

Another example of algorithm that is suitable for determining sequence similarity is the BLAST algorithm, which is described in Altschul et al, J. Mol. Biol. 215: 403-410 (1990). Software for performing BLAST analyses is publicly available through the National Center for Biotechnology Information, http://www.ncbi.nlm.nih.gov/; see also Zhang, Genome Res. 7:649-656 (1997) for the “PowerBLAST” variation. This algorithm involves first identifying high scoring sequence pairs (HSPs) by identifying short words of length W in the query sequence that either match or satisfy some positive valued threshold score T when aligned with a word of the same length in a database sequence. T is referred to as the neighborhood word score threshold (Altschul et al, J. Mol. Biol. 215: 403-410 (1990)). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a wordlength (W) of 11, the BLOSUM62 scoring matrix (see Henikoff, Proc. Natl. Acad. Sci. USA 89:10915-10919(1992)) alignments (B) of 50, expectation (E) of 10, M=5, N=−4, and a comparison of both strands. The term BLAST refers to the BLAST algorithm which performs a statistical analysis of the similarity between two sequences; see, e.g., Karlin, Proc. Natl. Acad. Sci. USA 90:5873-5787 (1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

In a preferred embodiment of the present invention, a nucleic acid molecule of the present invention encodes the homologue of a known protein. Table 1 sets forth a list of nucleic acid molecules that encode Chlorella sarokiniana proteins or fragments thereof which are homologues of known proteins

In a preferred embodiment of the present invention, a Chlorella sarokiniana protein or fragment thereof of the present invention is a protein homologue of another alga. In another preferred embodiment of the present invention, a Chlorella sarokiniana protein or fragment thereof of the present invention is a homologue of a fungal protein. In another preferred embodiment of the present invention, a Chlorella sarokiniana protein or fragment thereof of the present invention is a homologue of a mammalian protein. In another preferred embodiment of the present invention, a Chlorella sarokiniana protein or fragment thereof of the present invention is a homologue of a bacterial protein. In another preferred embodiment of the present invention, a Chlorella sarokiniana protein or fragment thereof of the present invention is a homologue of an insect protein. In another preferred embodiment of the present invention, a Chlorella sarokiniana protein or fragment thereof of the present invention is a homologue of a plant protein.

In a preferred embodiment of the present invention, the nucleic molecule of the present invention encodes a Chlorella sarokiniana protein or fragment thereof where a Chlorella sarokiniana protein or fragment thereof exhibits a BLAST probability score of greater than 1E-12, preferably a BLAST probability score of between about 1E-30 and about 1E-12, even more preferably a BLAST probability score of greater than 1E-30 with its homologue.

In another preferred embodiment of the present invention, the nucleic acid molecule encoding a Chlorella sarokiniana protein or fragment thereof exhibits a percent identity with its homologue of between about 25% and about 40%, more preferably of between about 40% and about 70%, even more preferably of between about 70% and about 90% and even more preferably between about 90% and 99%. In another preferred embodiment, of the present invention, a Chlorella sarokiniana protein or fragment thereof exhibits a percent identity with its homologue of 100%.

In a preferred embodiment of the present invention, the nucleic molecule of the present invention encodes a Chlorella sarokiniana protein or fragment thereof where the Chlorella sarokiniana protein exhibits a BLAST score of greater than 120, preferably a BLAST score of between about 1450 and about 120, even more preferably a BLAST score of greater than 1450 with its homologue.

The degeneracy of the genetic code, which allows different nucleotide sequences to code for the same protein or peptide, is known in the literature. (U.S. Pat. No. 4,757,006).

In an aspect of the present invention, one or more of the nucleic acid molecules of the present invention differ in nucleotide sequence from those encoding a Chlorella sarokiniana protein or fragment thereof in SEQ ID NO: 1 through SEQ ID NO: 9395 due to the degeneracy in the genetic code in that they encode the same protein but differ in nucleotide sequence.

In another further aspect of the present invention, one or more of the nucleic acid molecules of the present invention differ in nucleotide sequence from those encoding a Chlorella sarokiniana protein or fragment thereof in SEQ ID NO: 1 through SEQ ID NO: 9395 due to fact that the different nucleotide sequence encodes a protein having one or more conservative amino acid changes. It is understood that codons capable of coding for such conservative amino acid substitutions are known in the art.

It is well known in the art that one or more amino acids in a native sequence can be substituted with another amino acid(s), the charge and polarity of which are similar to that of the native amino acid, i.e., a conservative amino acid substitution, resulting in a silent change. Biologically functional equivalents of the proteins or fragments thereof of the present invention can have 10 or fewer conservative amino acid changes, more preferably seven or fewer conservative amino acid changes, and most preferably five or fewer conservative amino acid changes. The encoding nucleotide sequence will thus have corresponding base substitutions, permitting it to encode biologically functional equivalent forms of the proteins or fragments of the present invention.

It is understood that certain amino acids may be substituted for other amino acids in a protein structure without appreciable loss of interactive binding capacity with structures such as, for example, antigen-binding regions of antibodies or binding sites on substrate molecules. Because it is the interactive capacity and nature of a protein that defines that protein's biological functional activity, certain amino acid sequence substitutions can be made in a protein sequence and, of course, its underlying DNA coding sequence and, nevertheless, obtain a protein with like or superior properties. It is thus contemplated by the inventors that various changes may be made in the amino acid sequences of the proteins or fragments of the present invention, or corresponding DNA sequences that encode said polypeptides, without appreciable loss of their biological utility or activity. It is understood that codons capable of coding for such amino acid changes are known in the art.

In making such changes, the hydropathic index of amino acids may be considered. The importance of the hydropathic amino acid index in conferring interactive biological function on a protein is generally understood in the art (Kyte and Doolittle, J. Mol. Biol. 157, 105-132 (1982)). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, J. Mol. Biol. 157, 105-132 (1982)); these are isoleucine (+4.5), valine (+4.2), leucine (+3.8), phenylalanine (+2.8), cysteine/cystine (+2.5), methionine (+1.9), alanine (+1.8), glycine (−0.4), threonine (−0.7), serine (−0.8), tryptophan (−0.9), tyrosine (−1.3), proline (−1.6), histidine (−3.2), glutamate (−3.5), glutamine (−3.5), aspartate (−3.5), asparagine (−3.5), lysine (−3.9), and arginine (−4.5).

In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, states that the greatest local average hydrophilicity of a protein, as govern by the hydrophilicity of its adjacent amino acids, correlates with a biological property of the protein.

As detailed in U.S. Pat. No. 4,554,101, the following hydrophilicity values have been assigned to amino acid residues: arginine (+3.0), lysine (+3.0), aspartate (+3.0±1), glutamate (+3.0±1), serine (+0.3), asparagine (+0.2), glutamine (+0.2), glycine (0), threonine (−0.4), proline (−0.5±1), alanine (−0.5), histidine (−0.5), cysteine (−1.0), methionine (−1.3), valine (−1.5), leucine (−1.8), isoleucine (−1.8), tyrosine (−2.3), phenylalanine (−2.5), and tryptophan (−3.4).

In making such changes, the substitution of amino acids whose hydrophilicity values are within ±2 is preferred, those which are within ±1 are particularly preferred, and those within ±0.5 are even more particularly preferred.

In a further aspect of the present invention, one or more of the nucleic acid molecules of the present invention differ in nucleotide sequence from those encoding a Chlorella sarokiniana protein or fragment thereof set forth in SEQ ID NO: 1 through SEQ ID NO: 9395 or fragment thereof due to the fact that one or more codons encoding an amino acid has been substituted for a codon that encodes a nonessential substitution of the amino acid originally encoded.

Isolation and identification of nucleic acids of the present invention are described in detail in Examples. All or a substantial portion of the nucleic acids of the present invention may be used to isolate cDNAs and nucleic acids encoding Chlorella sarokiniana protein homologues or fragments thereof from the same or other species.

A “substantial portion” of a nucleotide sequence comprises enough of the sequence to afford specific identification and/or isolation of a nucleic acid fragment comprising the sequence. In general, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

Isolation of nucleic acids encoding protein homologues using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, structural nucleic acids encoding a Chlorella sarokiniana protein or fragment thereof, either as cDNAs or genomic DNAs, could be isolated directly by using all or a portion of the nucleic acids of the present invention as DNA hybridization probes to screen cDNA or genomic libraries from any desired species employing methodology well known to those skilled in the art. Methods for forming such libraries are well known in the art. Specific oligonucleotide probes based upon the nucleic acids of the present invention can be designed and synthesized by methods known in the art. Moreover, the entire sequences of the nucleic acids can be used directly to synthesize DNA probes by methods known to the skilled artisan such as random primer DNA labeling, nick translation, or end-labeling techniques, or RNA probes using available in vitro transcription systems. In addition, specific primers can be designed and used to amplify a part or all of the sequences. The resulting amplification products can be labeled directly during amplification reactions or labeled after amplification reactions, and used as probes to isolate full length cDNA or genomic DNAs under conditions of appropriate stringency.

Alternatively, the nucleic acids of interest can be amplified from nucleic acid samples using amplification techniques. For instance, the disclosed nucleic acids may be used to define a pair of primers that can be used with the polymerase chain reaction (Mullis, et al., Cold Spring Harbor Symp. Quant. Biol. 51:263-273 (1986); Erlich et al., EP 50,424; EP 84,796, EP 258,017, EP 237,362; Mullis, EP 201,184; Mullis et al., U.S. Pat. No. 4,683,202; Erlich, U.S. Pat. No. 4,582,788; and Saiki, R. et al., U.S. Pat. No. 4,683,194) to amplify and obtain any desired nucleic acid or fragment directly from mRNA, from cDNA, from genomic libraries or cDNA libraries. PCR and other in vitro amplification methods may also be useful, for example, to clone nucleic in acid sequences that code for proteins to be expressed, to make nucleic acids to use as probes for detecting the presence of the desired mRNA in samples, for nucleic acid sequencing, or for other purposes.

In addition, two short segments of the nucleic acids of the present invention may be used in polymerase chain reaction protocols to amplify longer nucleic acids encoding Chlorella sarokiniana protein homologues from DNA or RNA. For example, the skilled artisan can follow the RACE protocol (Frohman et al., Proc. Natl. Acad. Sci. USA 85:8998 (1988)) to generate cDNAs by using PCR to amplify copies of the region between a single point in the transcript and the 3′ or 5′ end. Primers oriented in the 3′ and 5′ directions can be designed from the nucleic acids of the present invention. Using commercially available 3′RACE or 5′RACE systems (Gibco BRL, Life Technologies, Gaithersburg, Maryland U.S.A.), specific 3′ or 5′ cDNA fragments can be isolated (Ohara et al., Proc. Natl. Acad. Sci. USA 86:5673 (1989); Loh et al., Science 243:217 (1989)). Products generated by the 3′ and 5′ RACE procedures can be combined to generate full-length cDNAs (Frohman and Martin, Techniques 1: 165 (1989)).

Nucleic acids of interest may also be synthesized, either completely or in part, especially where it is desirable to provide plant-preferred sequences, by well-known techniques as described in the technical literature. See, e.g., Carruthers et al., Cold Spring Harbor Symp. Quant. Biol. 47:411-418 (1982), and Adams et al., J. Am. Chem. Soc. 105:661 (1983). Thus, all or a portion of the nucleic acids of the present invention may be synthesized using codons preferred by a selected host. Species-preferred codons may be determined, for example, from the codons used most frequently in the proteins expressed in a particular host species. Other modifications of the nucleotide sequences may result in mutants having slightly altered activity.

Availability of the nucleotide sequences encoding Chlorella sarokiniana proteins or fragments thereof facilitates immunological screening of cDNA expression libraries. Synthetic polypeptides representing portions of the amino acid sequences of Chlorella sarokiniana proteins or fragments thereof may be synthesized. These polypeptides can be used to immunize animals to produce polyclonal or monoclonal antibodies with specificity for polypeptides or proteins comprising the amino acid sequences. These antibodies can be then used to screen cDNA expression libraries to isolate full-length cDNA clones of interest (Lemer, Adv. ImmunoL 36: 1 (1984); Sambrook et al., Molecular Cloning: A Laboratory Manual; Cold Spring Harbor Laboratory Press: Cold Spring Harbor, (1989)). It is understood that people skilled in the art are familiar with the standard resource materials which describe specific conditions and procedures for the construction, manipulation and isolation of antibodies (see, for example, Harlow and Lane, In Antibodies: A Laboratory Manual, Cold Spring Harbor Press, Cold Spring Harbor, N.Y. (1988)).

Another aspect of the present invention relates to a method for obtaining a nucleic acid comprising a nucleotide sequence encoding a Chlorella sarokiniana protein homologue. In a preferred embodiment, the method of the present invention for obtaining a nucleic acid encoding all or a substantial portion of the amino acid sequence of a Chlorella sarokiniana protein comprising: (a) probing a cDNA or genomic library with a hybridization probe comprising all or a portion of a nucleotide sequence selected from the group consisting of SEQ ID NO: 1 through SEQ ID NO:9395; (b) identifying a DNA clone that hybridizes with the hybridization probe; (c) isolating the DNA clone identified in step (b); and (d) sequencing the cDNA or genomic fragment that comprises the clone isolated in step (c) wherein the sequenced nucleic acid molecule encodes all or a substantial portion of the amino acid sequence of the Chlorella sarokiniana protein homologue.

In another preferred embodiment, the method of the present invention for obtaining a nucleic acid fragment encoding a substantial portion of the amino acid sequence of a Chlorella sarokiniana protein homologue comprising: (a) synthesizing a first and a second oligonucleotide primers corresponding to a portion of one of the sequences set forth in SEQ ID NO: 1 through SEQ ID NO: 9395; and (b) amplifying a cDNA insert present in a cloning vector using the first and second oligonucleotide primers of step (a) wherein the amplified nucleic acid molecule encodes all or a substantial portion of the amino acid sequence of the Chlorella sarokiniana protein homologue.

(b) Protein and Peptide Molecules

A class of agents comprises one or more of the substantially purified protein or peptide molecules encoded by SEQ ID NO: 1 through SEQ ID NO:9395 or one or more of the protein or fragment thereof or peptide molecules encoded by other nucleic acid agents of the present invention. Protein and peptide molecules can be identified using known protein or peptide molecules as a target sequence or target motif in the BLAST programs of the present invention. In a preferred embodiment the protein or fragment molecules of the present invention are derived from Chlorella sarokiniana.

The term “substantially purified protein or peptide molecule”, as used herein, refers to a protein or peptide molecule separated from substantially all other molecules normally associated. with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture.

The term “polypeptide”, “peptide”, or “protein”, as used herein, refers to a polymer composed of amino acids connected by peptide bonds. The term applies to any amino acid polymers in which one or more amino acid residue is an artificial chemical analogue of a corresponding naturally occurring amino acid, as well as to any naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. It is well known in the art that proteins or polypeptides may undergo modification, including but not limited to, disulfide bond formation, gamma-carboxylation of glutamic acid residues, glycosylation, lipid attachment, phosphorylation, oligomerization, hydroxylation and ADP-ribosylation. Exemplary modifications are described in most basic texts, such as, for example, Proteins—Structure and Molecular Properties, 2nd ed., T. E. Creighton, W. H. Freeman and Company, New York (1993). Many detailed reviews are available on this subject, such as, for example, those provided by Wold, F., Post-translational Protein Modifications. Perspectives and Prospects, pp. 1-12 in Post-translational Covalent Modification of Proteins, B. C. Johnson, Ed., Academic Press, New York (1983); Seifter et al., Meth. Enzymol. 182:626-M (1990) and Rattan et al., Protein Synthesis: Post-translational Modifications and Aging, Ann. N.Y. Acad. Sci. 663:48-62 (1992). Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E coli or other cells, prior to proteolytic processing, almost invariably will be N-formylmethionine. During post-translational modification of the polypeptide, a methionine residue at the NH₂ terminus may be deleted. Accordingly, this invention contemplates the use of both the methionine-containing and the methionin-less amino terminal variants of the protein of the invention. Thus, as used herein, the term “protein” or “polypeptide” includes any protein or polypeptide that is modified by any biological or non-biological process. The terms “amino acid” and “amino acids” refer to all naturally occurring amino acids and, unless otherwise limited, known analogs of natural amino acids that can function in a similar manner as naturally occurring amino acids. This definition is meant to include norleucine, omithine, homocysteine, and homoserine.

One or more of the protein or fragment of peptide molecules may be produced via chemical synthesis, or more preferably, by expressing in a suitable bacterial or eukaryotic host. Suitable methods for expression are described by Sambrook, et al., (In: Molecular Cloning, A Laboratory Manual, 2nd Edition, Cold Spring Harbor Press, Cold Spring Harbor, N.Y. (1989)), or similar texts.

A “protein fragment” is a peptide or polypeptide molecule whose amino acid sequence comprises a subset of the amino acid sequence of that protein. A protein or fragment thereof that comprises one or more additional peptide regions not derived from that protein is a “fusion” protein. Such molecules may be derivatized to contain carbohydrate or other moieties (such as keyhole limpet hemocyanin, etc.). Fusion proteins or peptide molecules of the present invention are preferably produced via recombinant means.

Another class of agents comprise protein or peptide molecules encoded by SEQ ID NO: 1 through SEQ ID NO:9395 or, fragments or fusions thereof in which non-essential, or not relevant, amino acid residues have been added, replaced, or deleted. Such a homologue can be obtained by any of a variety of methods. Most preferably, as indicated above, one or more of the disclosed sequences (e.g., SEQ ID NO: 1 through SEQ ID NO:9395 or complements thereof) will be used to define a pair of primers that may be used to isolate the homologue-encoding nucleic acid molecules from any desired species. Such molecules can be expressed to yield homologues by recombinant means.

(c) Antibodies

One aspect of the present invention concerns antibodies, single-chain antigen binding molecules, or other proteins that specifically bind to one or more of the protein or peptide molecules of the present invention and their homologues, fusions or fragments. Such antibodies may be used to quantitatively or qualitatively detect the protein or peptide molecules of the present invention. As used herein, an antibody or peptide is said to “specifically bind” to a protein or peptide molecule of the present invention if such binding is not competitively inhibited by the presence of non-related molecules. In a preferred embodiment the antibodies of the present invention bind to proteins of the present invention. In a more preferred embodiment the antibodies of the present invention bind to proteins derived from Chlorella sarokiniana.

Nucleic acid molecules that encode all or part of the protein of the present invention can be expressed, via recombinant means, to yield protein or peptides that can in turn be used to elicit antibodies that are capable of binding the expressed protein or peptide. Such antibodies may be used in immunoassays for that protein. Such protein-encoding molecules, or their fragments may be a “fusion” molecule (i.e., a part of a larger nucleic acid molecule) such that, upon expression, a fusion protein is produced. It is understood that any of the nucleic acid molecules of the present invention may be expressed, via recombinant means, to yield proteins or peptides encoded by these nucleic acid molecules.

The antibodies that specifically bind proteins and protein fragments of the present invention may be polyclonal or monoclonal, and may comprise intact immunoglobulins, or antigen binding portions of immunoglobulins (such as (F(ab′), F(ab′)₂) fragments, or single-chain immunoglobulins producible, for example, via recombinant means). It is understood that practitioners are familiar with the standard resource materials which describe specific conditions and procedures for the construction, manipulation and isolation of antibodies (see, for example, Harlow and Lane, In Antibodies: A Laboratory Manual, Cold Spring Harbor Press, Cold Spring Harbor, N.Y. (1988)).

As discussed below, such antibody molecules or their fragments may be used for diagnostic purposes. Where the antibodies are intended for diagnostic purposes, it may be desirable to derivatize them, for example with a ligand group (such as biotin) or a detectable marker group (such as a fluorescent group, a radioisotope or an enzyme).

The ability to produce antibodies that bind the protein or peptide molecules of the present invention permits the identification of mimetic compounds of those molecules. A “mimetic compound” is a compound that is not that compound, or a fragment of that compound, but which nonetheless exhibits an ability to specifically bind to antibodies directed against that compound.

It is understood that any of the agents of the present invention can be substantially purified and/or be biologically active and/or recombinant.

(d) Plant Constructs and Plant Transformants

The present invention also relates to a plant recombinant vector or construct comprising a structural nucleotide sequence encoding a C. sarokiniana protein or fragment thereof. The present invention also relates to a transformed plant cell or plant comprising in its genome an exogenous nucleic acid molecule encoding one or more C. sarokiniana proteins or fragments thereof. The present invention also relates to methods for creating a transgenic plant in which one or more C. sarokiniana proteins or fragments thereof are overexpressed.

By “exogenous” it is meant that a nucleic acid originates from outside a species into which the nucleic acid is introduced. An exogenous nucleic acid molecule can have a naturally occurring or non-naturally occurring nucleotide sequence. One skilled in the art understands that an exogenous nucleic acid molecule can be a heterologous nucleic acid derived from a different species than the species into which the nucleic acid is introduced or can be a nucleic acid derived from the same species as the species into which the nucleic acid is introduced.

The term “overexpression” refers to the expression of a polypeptide or protein encoded by an exogenous nucleic acid introduced into a host cell, wherein said polypeptide or protein is either not normally present in the-host cell, or wherein said polypeptide or protein thereof is present in said host cell at a higher level than that normally expressed from the endogenous gene encoding said polypeptide or protein. By “endogenous gene” refers to a native gene in its natural location in the genome of an organism.

The term “genome” as it applies to plant cells encompasses not only chromosomal DNA found within the nucleus, but organelle DNA found within subcellular components of the cell. DNAs of the present invention introduced into plant cells can therefore be either chromosomally integrated or organelle-localized. The term “genome” as it applies to bacteria encompasses both the chromosome and plasmids within a bacterial host cell. Encoding DNAs of the present invention introduced into bacterial host cells can therefore be either chromosomally integrated or plasmid-localized.

Method which are well known to those skilled in the art may be used to construct the plant recombinant construct or vector of the present invention. These method include in vitro recombinant DNA techniques, synthetic techniques, and in vivo genetic recombination. Such techniques are described in Sambrook et al., Molecular Cloning, A Laboratory Manual, Cold Spring Harbor Press, Plainview, N.Y. (1989); and Ausubel et al., Current Protocols in Molecular Biology, John Wiley & Sons, New York, N.Y. (1989).

A plant recombinant construct or vector of the present invention contains a structural nucleotide sequence encoding one or more C. sarokiniana proteins or fragments thereof and operably linked regulatory sequences or control elements.

The term “operably linked”, as used in reference to a regulatory sequence and a structural nucleotide sequence, means that the regulatory sequence causes regulated expression of the operably linked structural nucleotide sequence. “Expression” refers to the transcription and stable accumulation of sense or antisense RNA derived from the nucleic acid of the present invention. Expression may also refer to translation of mRNA into a polypeptide or protein. “Sense” RNA refers to RNA transcript that includes the mRNA and so can be translated into protein by the cell. “Antisense RNA” refers to a RNA transcript that is complementary to all or part of a target primary transcript or mRNA and that blocks the expression of a target gene (U.S. Pat. No. 5,107,065, incorporated herein by reference). The complementarity of an antisense RNA may be with any part of the specific gene transcript, i.e., at the 5′ non-coding sequence, 3′ non-translated sequence, a introns, or the coding sequence. “RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA.

“Regulatory sequences” or “control elements” refer to nucleotide sequences located upstream (5′ noncoding sequences), within, or downstream (3′ non-translated sequences) of a structural nucleotide sequence, and which influence the transcription, RNA processing or stability, or translation of the associated structural nucleotide sequence. Regulatory sequences may include promoters, translation leader sequences, introns, and polyadenylation recognition sequences.

The promoter sequence may consist of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions.

Promoters which are known or are found to cause transcription of DNA in plant cells can be used in the present invention. Such promoters may be obtained from a variety of sources such as plants and plant viruses. A number of promoters, including constitutive promoters, inducible promoters and tissue-specific promoters, that are active in plant cells have been described in the literature. It is preferred that the particular promoter selected should be capable of causing sufficient expression to result in the production of an effective amount of a protein to cause the desired phenotype. In addition to promoters that are known to cause transcription of DNA in plant cells, other promoters may be identified for use in the current invention by screening a plant cDNA library for genes that are selectively or preferably expressed in the target tissues and then determine the promoter regions.

The term “constitutive promoter” means a regulatory sequence which causes expression of a structural nucleotide sequence in most cells or tissues at most times. Constitutive promoters are active under most environmental conditions and states of development or cell differentiation. A variety of constitutive promoters are well known in the art. Examples of constitutive promoters that are active in plant cells include but are not limited to the nopaline synthase (NOS) promoters; the cauliflower mosaic virus (CaMV) 19S and 35S; the tobacco mosaic virus promoter; the figwort mosaic virus promoters; and actin promoters, such as the Arabidopsis actin gene promoter (see, e.g., Huang, Plant Mol. Biol. 33:125-139 (1997)).

The term “inducible promoter” refers to a regulatory sequence which causes conditional expression of a structural nucleotide sequence under the influence of changing environmental conditions or developmental conditions. Examples of inducible promoters include but are not limited to the light-inducible promoter from the small subunit of ribulose-1,5-bis-phosphate carboxylase (ssRUBISCO); the drought-inducible promoter of maize (Busk, Plant J. 11: 1285-1295 (1997)); the cold, drought, and high salt inducible promoter from potato (Kirch, Plant Mol. Biol. 33:897-909 (1997)); a nitrate-inducible promoter derived from the spinach nitrite reductase gene (Back et al., Plant Mol. Biol. 17:9 (1991)); salicylic acid inducible promoter (Uknes et al., Plant Cell 5:159-169 (1993); Bi et al., Plant J. 8:235-245 (1995)); the auxin-response elements E1 promoter fragment (AuxREs) in the soybean (Glycine max L.) (Liu, Plant Physiol. 115 :397-407 (1997)); the auxin-responsive Arabidopsis GST6 promoter (also responsive to salicylic acid and hydrogen peroxide) (Chen, Plant J. 10: 955-966 (1996)); the auxin-inducible parC promoter from tobacco (Sakai, 37:906-913 (1996)); a plant biotin response element (Streit, Mol. Plant Microbe Interact. 10:933-937 (1997)); the promoter responsive to the stress hormone abscisic acid (Sheen, Science 274:1900-1902 (1996)); the maize In2-2 promoter activated by benzenesulfonamide herbicide safeners (De Veylder, Plant Cell Physiol. 38:568-577 (1997)); a tetracycline-inducible promoter, such as the promoter for the Avena sativa L. (oat) arginine decarboxylase gene (Masgrau, Plant J. 11:465-473 (1997)); and a salicylic acid-responsive element (Stange, Plant J. 11: 1315-1324 (1997)).

The term “tissue-specific promoter” means a regulatory sequence that causes transcriptions or enhanced transcriptions of DNA in specific cells or tissues at specific times during plant development, such as in vegetative tissues or reproductive tissues. Examples of tissue-specific promoters under developmental control include promoters that initiate transcription only (or primarily only) in certain tissues, such as vegetative tissues, e.g., roots, leaves or stems, or reproductive tissues, such as fruit, ovules, seeds, pollen, pistols, flowers, or any embryonic tissue. Reproductive tissue specific promoters may be, e.g., ovule-specific, embryo-specific, endosperm-specific, integument-specific, seed coat-specific, pollen-specific, petal-specific, sepal-specific, or some combination thereof. One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

A variety of promoters specifically active in vegetative tissues, such as leaves, stems, roots and tubers, can also be used to express the nucleic acids of the invention. Examples of tuber-specific promoters include but are not limited to the class I and II patatin promoters (Bevan et al., EMBO J. 8: 1899-1906 (1986); Koster-Topfer et al., Mol Gen Genet. 219: 390-396 (1989); Mignery et al., Gene. 62: 27-44 (1988); Jefferson et al., Plant Mol. Biol. 14: 995-1006 (1990)), the promoter for the potato tuber ADPGPP genes, both the large and small subunits; the sucrose synthase promoter (Salanoubat and Belliard, Gene. 60: 47-56 (1987), Salanoubat and Belliard, Gene. 84: 181-185 (1989)); and the promoter for the major tuber proteins including the 22 kd protein complexes and proteinase inhibitors (Hannapel, Plant Physiol. 101: 703-704 (1993)). Examples of leaf-specific promoters include but are not limited to the ribulose biphosphate carboxylase (RBCS or RuBISCO) promoters (see, e.g., Matsuoka, Plant J. 6:311-319 (1994)); the light harvesting chlorophyll a/b binding protein gene promoter (see, e.g., Shiina, Plant Physiol. 115-.477-483 (1997); Casal, Plant Physiol. 116:1533-1538 (1998)); and the Arabidopsis thaliana myb-related gene promoter (Atmyb5) (Li, FEBS Lett. 379:117-121 (1996)). Examples of root-specific promoter include but are not limited to the promoter for the acid chitinase gene (Samac et al., Plant Mol. Biol. 25: 587-596 (1994)); the root specific subdomains of the CaMV35S promoter that have been identified (Lam et al., Proc. Natl. Acad. Sci. (U.S.A.) 86:7890-7894 (1989)); the ORF1 3 promoter from Agrobacterium rhizogenes which exhibits high activity in roots (Hansen, Mol. Gen. Genet. 254:337-343 (1997)); the promoter for the tobacco root-specific gene TobRB7 (Yamamoto, Plant Cell 3:371-382 (1991)); and the root cell specific promoters reported by Conkling et al. (Conkling et al., Plant Physiol. 93:1203-1211 (1990)).

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems (Di Laurenzio, Cell 86:423-433 (1996); Long, Nature 379:66-69 (1996)), can be used. Another example of a useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto, Plant Cell. 7:517-527 (1995)). Also another example of a useful promoter is that which controls the expression of knl-related genes from maize and other species which show meristem-specific expression (see, e.g., Granger, Plant Mol. Biol. 31:373-378 (1996); Kerstetter, Plant Cell 6:1877-1887 (1994); Hake, Philos. Trans. R. Soc. Lond. B. Biol. Sci. 350:45-51 (1995). Another example of a meristematic promoter is the Arabidopsis thaliana KNAT1 promoter. In the shoot apex, KNAT1 transcript is localized primarily to the shoot apical meristem; the expression of KNATI in the shoot meristem decreases during the floral transition and is restricted to the cortex of the inflorescence stem (see, e.g., Lincoln, Plant Cell 6:1859-1876 (1994)).

Suitable seed-specific promoters can be derived from the following genes: MAC1 from maize (Sheridan, Genetics 142:1009-1020 (1996); Cat3 from maize (GenBank No. L05934, Abler, Plant Mol. Biol. 22:10131-1038 (1993); vivparous-1 from Arabidopsis (GenBank No. U93215); Atimycl from Arabidopsis (Urao, Plant Mol. Biol. 32:571-57 (1996); Conceicao, Plant 5:493-505 (1994); napA from Brassica napus (GenBank No. J02798); the napin gene family from Brassica napus (Sjodahl, Planta 197:264-271 (1995)).

The ovule-specific BEL1 gene described in Reiser (1995) Cell 83:735-742, GenBank No. U39944, can also be used. See also Ray (1994) Proc. Natl. Acad. Sci. USA 91:5761-5765. The egg and central cell specific FIEEI promoter is also a useful reproductive tissue-specific promoter.

A maize pollen-specific promoter has been identified in maize (Guerrero (1990) Mol. Gen. Genet. 224:161-168). Other genes specifically expressed in pollen are described, e.g., by Wakeley (1998) Plant Mol. Biol. 37:187-192; Ficker (1998) Mol. Gen. Genet. 257:132-142; Kulikauskas (1997) Plant Mol. B 34:809-814; Treacy (1997) Plant Mol. Biol. 34:603-611.

Promoters derived from genes encoding embryonic storage proteins, which includes the gene encoding the 2S storage protein from Brassica napus (Dasgupta, Gene 133:301-302 (1993); the 2s seed storage protein gene family from Arabidopsis; the gene encoding oleosin 2OkD from Brassica napus (GenBank No. M63985); the genes encoding oleosin A (GenBank No. U09118) and oleosin B (GenBank No. U09119) from soybean; the gene encoding oleosin from Arabidopsis (GenBank No. Z 17657); the gene encoding oleosin 18 kD from maize (GenBank No. J05212, Lee, Plant Mol. Biol. 26:1981-1987 (1994)); and the gene encoding low molecular weight sulphur rich protein from soybean (Choi, Mol Gen, Genet. 246:266-268 (1995)), can also be used.

Promoters derived from genes encoding for zein genes (including the 15 kD, 16 kD, 19 kD, 22 kD, 27 kD, and-gamma genes) (Pedersen et al., Cell 29: 1015-1026 (1982)) can be also used. The zeins are a group of storage proteins found in maize endosperm.

Other promoters known to function, for example, in maize, include the promoters for the following genes: waxy, Brittle, Shrunken 2, Branching enzymes I and II, starch synthases, debranching enzymes, oleosins, glutelins, and sucrose synthases. A particularly preferred promoter for maize endosperm expression is the promoter for the glutelin gene from rice, more particularly the Osgt-I promoter (Zheng et al., Mol. Cell Biol. 13: 5829-5842 (1993), herein incorporated by reference in its entirety). Examples of promoters suitable for expression in wheat include those promoters for the ADPglucose pyrophosphorylase (ADPGPP) subunits, the granule bound and other starch synthases, the branching and debranching enzymes, the embryogenesis-abundant proteins, the gliadins, and the glutenins. Examples of such promoters in rice include those promoters for the ADPGPP subunits, the granule bound and other starch synthases, the branching enzymes, the debranching enzymes, sucrose synthases, and the glutelins. A particularly preferred promoter is the promoter for rice glutelin, Osgt-1. Examples of such promoters for barley include those for the ADPGPP subunits, the granule bound and other starch synthases, the branching enzymes, the debranching enzymes, sucrose synthases, the hordeins, the embryo globulins, and the aleurone specific proteins.

A tomato promoter active during fruit ripening, senescence and abscission of leaves and, to a lesser extent, of flowers can be used (Blume, Plant J. 12:731-746 (1997)). Other exemplary promoters include the pistol specific promoter in the potato (Solarium tuberosum L.) SK2 gene, encoding a pistil-specific basic endochitinase (Ficker, Plant Mol. Biol. 35:425-431 (1997)); the Blec4 gene from pea (Pisum sativum cv. Alaska), active in epidermal tissue of vegetative and floral shoot apices of transgenic alfalfa. This makes it a useful tool to target the expression of foreign genes to the epidermal layer of actively growing shoots. The tissue specific E8 promoter from tomato is also useful for directing gene expression in fruits.

It is recognized that additional promoters that may be utilized are described, for example, in U.S. Pat. Nos. 5,378,619, 5,391,725, 5,428,147, 5,447,858, 5,608,144, 5,608,144, 5,614,399, 5,633,441, 5,633,435, and 4,633,436, all of which are herein incorporated in their entirety. In addition, a tissue specific enhancer may be used (Fromm et al., The Plant Cell 1:977-984 (1989), herein incorporated by reference in its entirety). It is further recognized that since in most cases the exact boundaries of regulatory sequences have not been completely defined, DNA fragments of different lengths may have identical promoter activity.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Molecular Biotechnology 3:225).

The “3′ non-translated sequences” refer to DNA sequences located downstream of a structural nucleotide sequence and include sequences encoding polyadenylation and other regulatory signals capable of affecting mRNA processing or gene expression. The polyadenylation signal functions in plants to cause the addition of polyadenylate nucleotides to the 3′ end of the mRNA precursor. The polyadenylation sequence can be derived from the natural gene, from a variety of plant genes, or from T-DNA. An example of the polyadenylation sequence is the nopaline synthase 3′ sequence (NOS 3′; Fraley et al., Proc. Natl. Acad. Sci. USA 80: 4803-4807 (1983)). The use of different 3′ non-translated sequences is exemplified by Ingelbrecht et al., Plant Cell 1:671-680 (1989).

Generally, optimal expression in monocotyledonous and some dicotyledonous plants is obtained when an intron sequence is inserted between the promoter sequence and the structural gene sequence or, optionally, may be inserted in the structural coding sequence to provide an interrupted coding sequence. An example of such an intron sequence is the HSP 70 intron described in WO 93/19189.

A recombinant vector or construct of the present invention will typically comprise a selectable marker which confers a selectable phenotype on plant cells. Selectable markers may also be used to select for plants or plant cells that contain the exogenous nucleic acids encoding polypeptides or proteins of the present invention. The marker may encode biocide resistance, antibiotic resistance (e.g., kanamycin, G418 bleomycin, hygromycin, etc.), or herbicide resistance (e.g., glyphosate, etc.). Examples of selectable markers include, but are not limited to, a neo gene (Potrykus et al., Mol. Gen. Genet. 199: 183-188 (1985)) which codes for kanamycin resistance and can be selected for using kanamycin, G418, etc.; a bar gene which codes for bialaphos resistance; a mutant EPSP synthase gene (Hinchee et al., Bio/Technology 6:915-922 (1988)) which encodes glyphosate resistance; a nitrilase gene which confers resistance to bromoxynil (Stalker et al., J. Biol. Chem. 263:6310-6314 (1988)); a mutant acetolactate synthase gene (ALS) which confers imidazolinone or sulphonylurea resistance (European Patent Application 154,204 (Sep. 11, 1985)); and a methotrexate resistant DHFR gene (Thillet et al., J. Biol. Chem. 263:12500-12508 (1988)).

A recombinant vector or construct of the present invention may also include a screenable marker.

Screenable markers may be used to monitor expression. Exemplary screenable markers include β-glucuronidase or uidA gene (GUS) which encodes an enzyme for which various chromogenic substrates are known (Jefferson, Plant Mol. Biol, Rep. 5:387-405 (1987); Jefferson et al., EMBO J. 6:3901-3907 (1987)); an R-locus gene, which encodes a product that regulates the production of anthocyanin pigments (red color) in plant tissues (Dellaporta et al., Stadler Symposium 11:263-282 (1988)); a β-lactamase gene (Sutcliffe et al., Proc. Natl. Acad. Sci. (U.S.A.) 75:3737-3741 (1978)), a gene which encodes an enzyme for which various chromogenic substrates are known (e.g., PADAC, a chromogenic cephalosporin); a luciferase gene (Ow et al., Science 234:856-859 (1986)) a xylE gene (Zukowsky et al., Proc. Natl. Acad. Sci. (USA.) 80:1101-1105 (1983)) which encodes a catechol dioxygenase that can convert chromogenic catechols; an α-amylase gene (Ikatu et al., Bio/Technol. 8:241-242 (1990)); a tyrosinase gene (Katz et al., J. Gen. Microbiol. 129:2703-2714 (1983)) which encodes an enzyme capable of oxidizing tyrosine to DOPA and dopaquinone which in turn condenses to melanin; an a-galactosidase, which will turn a chromogenic α-galactose substrate.

Included within the terms “selectable or screenable marker genes” are also genes which encode a secretable marker whose secretion can be detected as a means of identifying or selecting for transformed cells. Examples include markers which encode a secretable antigen that can be identified by antibody interaction, or even secretable enzymes which can be detected catalytically. Secretable proteins fall into a number of classes, including small, diffusible proteins detectable, e.g., by ELISA, small active enzymes detectable in extracellular solution (e.g., α-amylase, β-lactamase, phosphinothricin transferase), or proteins which are inserted or trapped in the cell wall (such as proteins which include a leader sequence such as that found in the expression unit of extension or tobacco PR-S). Other possible selectable and/or screenable marker genes will be apparent to those of skill in the art.

In addition to a selectable marker, it may be desirous to use a reporter gene. In some instances a p reporter gene may be used with or without a selectable marker. Reporter genes are genes which are typically not present in the recipient organism or tissue and typically encode for proteins resulting in some phenotypic change or enzymatic property. Examples of such genes are provided in K. Wising et al. Ann. Rev. Genetics, 22, 421 (1988), which is incorporated herein by reference. Preferred reporter genes include the beta-glucuronidase (GUS) of the uidA locus of E. coli, the chloramphenicol acetyl transferase gene from Tn9 of E. coli, the green fluorescent protein from the bioluminescent jellyfish Aequorea victoria, and the luciferase genes from firefly Photinus pyralis. An assay for detecting reporter gene expression may then be performed at a suitable time after said gene has been introduced into recipient cells. A preferred such assay entails the use of the gene encoding beta-glucuronidase (GUS) of the uidA locus of E. coli as described by Jefferson et al., (1987 Biochem. Soc. Trans. 15, 17-19) to identify transformed cells.

In preparing the DNA constructs of the present invention, the various components of the construct or fragments thereof will normally be inserted into a convenient cloning vector, e.g., a plasmid that is capable of replication in a bacterial host, e.g., E. coli. Numerous vectors exist that have been described in the literature, many of which are commercially available. After each cloning, the cloning vector with the desired insert may be isolated and subjected to further manipulation, such as restriction digestion, insertion of new fragments or. nucleotides, ligation, deletion, mutation, resection, etc. so as to tailor the components of the desired sequence. Once the construct has been completed, it may then be transferred to an appropriate vector for further manipulation in accordance with the manner of transformation of the host cell.

A recombinant vector or construct of the present invention may also include a chloroplast transit peptide, in order to target the polypeptide or protein of the present invention to the plastid. The term “plastid” refers to the class of plant cell organelles that includes amyloplasts, chloroplasts, chromoplasts, elaioplasts, eoplasts, etioplasts, leucoplasts, and proplastids. These organelles are self-replicating, and contain what is commonly referred to as the “chloroplast genome,” a circular DNA molecule that ranges in size from about 120 to about 217 kb, depending upon the plant species, and which usually contains an inverted repeat region. Many plastid-localized proteins are expressed from nuclear genes as precursors and are targeted to the plastid by a chloroplast transit peptide (CTP), which is removed during the import steps. Examples of such chloroplast proteins include the small subunit of ribulose-1,5-biphosphate carboxylase (ssRUBISCO, SSU), 5-enolpyruvateshikimate-3-phosphate synthase (EPSPS), ferredoxin, ferredoxin oxidoreductase, the light-harvesting-complex protein I and protein II, and thioredoxin F. It has been demonstrated that non-plastid proteins may be targeted to the chloroplast by use of protein fusions with a CTP and that a CTP sequence is sufficient to target a protein to the plastid. Those skilled in the art will also recognize that various other chimeric constructs can be made that utilize the functionality of a particular plastid transit peptide to import the enzyme into the plant cell plastid depending on the promoter tissue specificity.

The present invention also provide a transgenic plant comprising in its genome an exogenous nucleic acid which comprises: (A) a 5′ non-coding sequence which functions in the cell to cause the production of a mRNA molecule; which is operably linked to (B) a structural nucleotide sequence, wherein the structural nucleotide sequence encodes a C. sarokiniana proteins or fragments thereof; which is operably linked to (C) a 3′ non-translated sequence that functions in said cell to cause termination of transcription.

The term “transgenic plant” refers to a plant that contains an exogenous nucleic acid, which can be derived from the same plant species or from a different plant species. Transgenic plants are also meant to comprise progeny (decendant, offspring, etc.) of any generation of such a transgenic plant. A seed of any generation of all such transgenic plants wherein said seed comprises a DNA sequence encoding the protein or fragment thereof of the present invention is also an important aspect of the invention.

The DNA constructs of the present invention may be introduced into the genome of a desired plant host by a variety of conventional transformation techniques, which are well known to those skilled in the art. Preferred methods of transformation of plant cells or tissues are the Agrobacterium mediated transformation method and the biolistics or particle-gun mediated transformation method. Suitable plant transformation vectors for the purpose of Agrobacterium mediated transformation include those derived from a Ti plasmid of Agrobacterium tumefaciens, as-well as those disclosed, e.g., by Herrera-Estrella et al., Nature 303:209 (1983); Bevan, Nucleic Acids Res. 12: 8711-8721 (1984); Klee et al., Bio-Technology 3(7): 637-642 (1985); and EPO publication 120,516. In addition to plant transformation vectors derived from the Ti or root-inducing (Ri) plasmids of Agrobacterium, alternative methods can be used to insert the DNA constructs of this invention into plant cells. Such methods may involve, but are not limited to, for example, the use of liposomes, electroporation, chemicals that increase free DNA uptake, free DNA delivery via microprojectile bombardment, and transformation using viruses or pollen.

A plasmid expression vector suitable for the introduction of a nucleic acid encoding a polypeptide or protein of the present invention in monocots using electroporation or particle-gun mediated transformation is composed of the following: a promoter that is constitutive or tissue-specific; an intron that provides a splice site to facilitate expression of the gene, such as the Hsp70 intron (PCT Publication WO93/19189); and a 3′ polyadenylation sequence such as the nopaline synthase 3′ sequence (NOS 3′; Fraley et al., Proc. Natl. Acad. Sci. USA 80: 4803-4807(1983)). This expression cassette may be assembled on high copy replicons suitable for the production of large quantities of DNA.

An example of a useful Ti plasmid cassette vector for plant transformation is pMON 17227. This vector is described in PCT Publication WO 92/04449 and contains a gene encoding an enzyme conferring glyphosate resistance (denominated CP4), which is an excellent selection marker gene for many plants. The gene is fused to the Arabidopsis EPSPS chloroplast transit peptide (CTP2) and expressed from the FMV promoter as described therein.

When adequate numbers of cells (or protoplasts) containing the exogenous nucleic acid encoding a polypeptide or protein of the present invention are obtained, the cells (or protoplasts) are regenerated into whole plants. Choice of methodology for the regeneration step is not critical, with suitable protocols being available for hosts from Leguminosae (alfalfa, soybean, clover, etc.), Umbelliferae (carrot, celery, parsnip), Cruciferae (cabbage, radish, canola/rapeseed, etc.), Cucurbitaceae (melons and cucumber), Gramineae (wheat, barley, rice, maize, etc.), Solanaceae (potato, tobacco, tomato, peppers), various floral crops, such as sunflower, and nut-bearing trees, such as almonds, cashews, walnuts, and pecans. See, for example, Ammirato et al., Handbook of Plant Cell Culture -Crop Species. Macmillan Publ. Co. (1984); Shimamoto et al., Nature 338:274-276 (1989); Fromm, UCLA Symposium on Molecular Strategies for Crop Improvement, Apr. 16-22, 1990. Keystone, Colo. (1990); Vasil et al., Bio/Technology 8:429-434 (1990); Vasil et al., Bio/Technology 10:667-674 (1992); Hayashimoto, Plant Physiol. 93:857-863 (1990); and Datta et al., Bio-technology 8:736-740 (1990). Regeneration can also be obtained from plant callus, explants, organs, or parts thereof. Such regenration techniques are described generally in Klee et al., Ann. Rev. Plant Phys. 38:467-486 (1987).

A transgenic plant formed using Agrobacterium transformation methods typically contains a single exogenous gene on one chromosome. Such transgenic plants can be referred to as being heterozygous for the added exogenous gene. More preferred is a transgenic plant that is homozygous for the added exogenous gene; i.e., a transgenic plant that contains two added exogenous genes, one gene at the same locus on each chromosome of a chromosome pair. A homozygous transgenic plant can be obtained by sexually mating (selfing) an independent segregant transgenic plant that contains a single exogenous gene, germinating some of the seed produced and analyzing the resulting plants produced for the exogenous gene of interest.

The development or regeneration of transgenic plants containing the exogenous nucleic acid that encodes a polypeptide or protein of interest is well known in the art. Preferably, the regenerated plants are self-pollinated to provide homozygous transgenic plants, as discussed above. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide or protein of the present invention is cultivated using methods well known to one skilled in the art.

Transgenic plants, that can be generated by practice of the present invention, include but are not limited to Acacia, alfalfa, aneth, apple, apricot, artichoke, arugula, asparagus, avocado, banana, barley, beans, beet, blackberry, blueberry, broccoli, brussels sprouts, cabbage, canola, cantaloupe, carrot, cassava, cauliflower, celery, cherry, cilantro, citrus, clementines, coffee, corn, cotton, cucumber, Douglas fir, eggplant, endive, escarole, eucalyptus, fennel, figs, gourd, grape, grapefruit, honey dew, jicama, kiwifruit, lettuce, leeks, lemon, lime, Loblolly pine, mango, melon, mushroom, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, raspberry, rice, rye, sorghum, Southern pine, soybean, spinach, squash, strawberry, sugarbeet, sugarcane, sunflower, sweet potato, sweetgum, tangerine, tea, tobacco, tomato, turf, a vine, watermelon, wheat, yams, and zucchini.

The present invention also provides parts of the transgenic plants of present invention. Plant parts, without limitation, include seed, endosperm, ovule and pollen. In a particularly preferred embodiment of the present invention, the plant part is a seed.

The present invention also further provides method for generating a transgenic plant comprising the steps of: a) introducing into the genome of the plant an exogenous nucleic acid, wherein the exogenous nucleic acid comprises in the 5′ to 3′ direction i) a promoter that functions in the cells of said plant, said promoter operably linked to; ii) a structural nucleic acid sequence encoding a C. sarokiniana protein or fragment thereof, said structural nucleic acid sequence operably linked to; iii) a 3′ non-translated nucleic acid sequence that functions in said cells of said plant to cause transcriptional termination; b) obtaining transformed plant cells containing the nucleic acid sequence of step (a); and c) regenerating from said transformed plant cells a transformed plant in which said polypeptide or protein is overexpressed.

Any of the isolated nucleic acid molecules of the present invention may be introduced into a plant cell in a permanent or transient manner in combination with other genetic elements such as vectors, promoters, enhancers etc. Further any of the nucleic acid molecules encoding a C. sarokiniana protein or fragment thereof may be introduced into a plant cell in a manner that allows for over expression of the protein or polypeptide encoded by the nucleic acid molecule.

Antibodies have been expressed in plants (Hiatt et al, Nature 342:76-78 (1989); Conrad and Fielder, Plant Mol Biol. 26:1023-1030 (1994)). Cytoplasmic expression of a scFv (single-chain Fv antibodies) has been reported to delay infection by artichoke mottled crinkle virus. Transgenic plants that express antibodies directed against endogenous proteins may exhibit a physiological effect (Philips et al, EMBO J. 16:4489-4496 (1997); Marion-Poll, Trends in Plant Science 2:447-448 (1997)). For example, expressed anti-abscisic antibodies reportedly result in a general perturbation of seed development (Philips et al, EMBO J. 16:4489-4496 (1997)).

Antibodies that are catalytic may also be expressed in plants (abzymes). The principle behind 3 abzymes is that since antibodies may be raised against many molecules, this recognition ability can be directed toward generating antibodies that bind transition states to force a chemical reaction forward (Persidas, Nature Biotechnology 15:1313-1315 (1997); Baca et al, Ann. Rev. Biophys. Biomol. Struct. 26:461-493 (1997)). The catalytic abilities of abzymes may be enhanced by site directed mutagenesis. Examples of abzymes are, for example, set forth in U.S. Pat. Nos: 5,658,753; 5,632,990; 5,631,137; 5,602,015; 5,559,538; 5,576,174; 5,500,358; 5,318,897; 5,298,409; 5,258,289 and 5,194,585, all of which are herein incorporated in their entirety.

It is understood that any of the antibodies of the present invention may be expressed in plants and that such expression can result in a physiological effect. It is also understood that any of the expressed antibodies may be catalytic.

(e) Algal Constructs and Algal Transformants

The present invention also relates to an algal recombinant vector comprising exogenous genetic material. The present invention also relates to an algal cell comprising an algal recombinant vector. The present invention also relates to methods for obtaining a recombinant algal host cell comprising introducing into an algal host cell exogenous genetic material.

Exogenous genetic material is any genetic material, whether naturally occurring or otherwise, from any source that is capable of being inserted into any organism. Exogenous genetic material may be transferred into an algal cell. In a preferred embodiment the exogenous genetic material includes a nucleic acid molecule having a sequence selected from the group consisting of SEQ ID NO: 1 through SEQ ID NO: 9395 or complements thereof.

The algal recombinant vector may be any vector which can be conveniently subjected to recombinant DNA procedures. The choice of a vector will typically depend on the compatibility of the vector with the algal host cell into which the vector is to be introduced. The vector may be a linear or a closed circular plasmid. The vector system may be a single vector or plasmid or two or more vectors or plasmids which together contain the total DNA to be introduced into the genome of the algal host.

The algal vector may be an autonomously replicating vector, ie., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the algal cell, is integrated into the genome and replicated together with the chromosome(s) into which it has been integrated. For integration, the vector may rely on the nucleic acid sequence of the vector for stable integration of the vector into the genome by homologous or nonhomologous recombination. Alternatively, the vector may contain additional nucleic acid sequences for directing integration by homologous recombination into the genome of the algal host. The additional nucleic acid sequences enable the vector to be integrated into the host cell genome at a precise location(s) in the chromosome(s). To increase the likelihood of integration at a precise location, there should be preferably two nucleic acid sequences which individually contain a sufficient number of nucleic acids, preferably 400 bp to 1500 bp, more preferably 800 bp to 1000 bp, which are highly homologous with the corresponding target sequence to enhance the probability of homologous recombination. These nucleic acid sequences may be any sequence that is homologous with a target sequence in the genome of the algal host cell, and, furthermore, may be non-encoding-or encoding sequences.

The vectors of the present invention preferably contain one or more selectable markers which permit easy selection of transformed cells. A selectable marker is a gene, the product of which confers upon an algal cell resistance to a compound to which the algal would otherwise be sensitive. The compound can be selected from the group consisting of antibiotics, fungicides, herbicides, and heavy metals. The selectable marker may be selected from any known or subsequently identified selectable markers, including markers derived from algal, fungal, and bacterial sources. Preferred selectable markers can be selected from the group including, but not limited to, amdS (acetamidase), argB (ornithine carbamoyltransferase), bar (phosphinothricin acetyltransferase), ble (bleomycin binding protein), cat (chloramphenicol acetyltransferase), hygB (hygromycin B phosphotransferase), nat (nourseothricin acetyltransferase), niaD (nitrate reductase), neo (neomycin phosphotransferase),pac (puromycin acetyltransferase), pyrG (orotidine-5′-phosphate decarboxylase), sat (streptothricin acetyltransferase), sC (sulfate adenyltransferase), trpC (anthranilate synthase), and glyphosate resistant EPSPS genes. Furthermore, selection may be accomplished by co-transformation, e.g., as described in WO 91/17243, herein incorporated by reference in its entirety.

A nucleic acid sequence of the present invention may be operably linked to a suitable promoter sequence. The promoter sequence is a nucleic acid sequence which is recognized by the algal host cell for expression of the nucleic acid sequence. The promoter sequence contains transcription and translation control sequences which mediate the expression of the protein or fragment thereof.

A promoter may be any nucleic acid sequence which shows transcriptional activity in the algal host cell of choice and may be obtained from genes encoding polypeptides either homologous or heterologous to the host cell. Examples of suitable promoters for directing the transcription of a nucleic acid construct of the invention in an algal host are light harvesting protein promoters obtained from photosynthetic organisms, Chlorella virus methyltransferase promoters, CaMV 35 S promoter, PL promoter from bacteriophage λ, nopaline synthase promoter from the Ti plasmid of Agrobacterium tumefaciens, and bacterial trp promotor.

A protein or fragment thereof encoding nucleic acid molecule of the present invention may also be operably linked to a terminator sequence at its 3′ terminus. The terminator sequence may be native to the nucleic acid sequence encoding the protein or fragment thereof or may be obtained from foreign sources. Any terminator which is functional in the algal host cell of choice may be used in the present invention.

A protein or fragment thereof encoding nucleic acid molecule of the present invention may also be operably linked to a suitable leader sequence. A leader sequence is a nontranslated region of a mRNA which is important for translation by the algal host. The leader sequence is operably linked to the 5′ terminus of the nucleic acid sequence encoding the protein or fragment thereof. The leader sequence may be native to the nucleic acid sequence encoding the protein or fragment thereof or may be obtained from foreign sources. Any leader sequence which is functional in the algal host cell of choice may be used in the present invention.

A polyadenylation sequence may also be operably linked to the 3′ terminus of the nucleic acid sequence of the present invention. The polyadenylation sequence is a sequence which when transcribed is recognized by the algal host to add polyadenosine residues to transcribed mRNA. The polyadenylation sequence may be native to the nucleic acid sequence encoding the protein or fragment thereof or may be obtained from foreign sources. Any polyadenylation sequence which is functional in the algal host of choice may be used in the present invention.

To avoid the necessity of disrupting the cell to obtain the protein or fragment thereof, and to minimize the amount of possible degradation of the expressed protein or fragment thereof within the cell, it is preferred that expression of the protein or fragment thereof gives rise to a product secreted outside the cell. To this end, the protein or fragment thereof of the present invention may be linked to a signal peptide linked to the amino terminus of the protein or fragment thereof. A signal peptide is an amino acid sequence which permits the secretion of the protein or fragment thereof from the algal host into the culture medium. The signal peptide may be native to the protein or fragment thereof of the invention or may be obtained from foreign sources. The 5′ end of the coding sequence of the nucleic acid sequence of the present invention may inherently contain a signal peptide coding region naturally linked in translation reading frame with the segment of the coding region which encodes the secreted protein or fragment thereof. Alternatively, the 5′ end of the coding sequence may contain a signal peptide coding region which is foreign to that portion of the coding sequence which encodes the secreted protein or fragment thereof. The foreign signal peptide may be required where the coding sequence does not normally contain a signal peptide coding region. Alternatively, the foreign signal peptide may simply replace the natural signal peptide to obtain enhanced secretion of the desired protein or fragment thereof. Any signal peptide capable of permitting secretion of the protein or fragment thereof in an algal host of choice may be used in the present invention.

A protein or fragment thereof encoding nucleic acid molecule of the present invention may also be linked to a propeptide coding region. A propeptide is an amino acid sequence found at the amino terminus of aproprotein or proenzyme. Cleavage of the propeptide from the proprotein yields a mature biochemically active protein. The resulting polypeptide is known as a propolypeptide or proenzyme (or a zymogen in some cases). Propolypeptides are generally inactive and can be converted to mature active polypeptides by catalytic or autocatalytic cleavage of the propeptide from the propolypeptide or proenzyme. The propeptide coding region may be native to the protein or fragment thereof or may be obtained from foreign sources. The foreign propeptide coding region may be obtained from the Saccharomyces cerevisiae alpha-factor gene or Myceliophthora thermophila laccase gene (WO 95/33836, herein incorporated by reference in its entirety).

The procedures used to ligate the elements described above to construct the recombinant expression vector of the present invention are well known to one skilled in the art (see, for example, Sambrook, 2nd ed., et al., Molecular Cloning, A Laboratory Manual Cold Spring Harbor, N.Y., (1989), herein incorporated by reference in its entirety).

The present invention also relates to recombinant algal host cells produced by the methods of the present invention which are advantageously used with the recombinant vector of the present invention. The cell is preferably transformed with a vector comprising a. nucleic acid sequence of the invention followed by integration of the vector into the host chromosome. The choice of algal host cells will to a large extent depend upon the gene encoding the protein or fragment thereof and its source.

Algal cells may be transformed by a variety of known techniques, including but not limit to, microprojectile bombardment, protoplast fusion, electroporation, microinjection, and vigorous agitation in the presence of glass beads. Suitable procedures for transformation of green algal host cells are described in EP 108 580, herein incorporated by reference in its entirety. A suitable method of transforming Chlorella species is described by Jarvis and Brown, Curr. Genet. 19: 317-321 (1991), herein incorporated by reference in its entirety. A suitable method of transforming cells of diatom Phaeodactylum tricornutum species is described in WO 97/39106, herein incorporated by reference in its entirety. Chlorophyll C-containing algae may be transformed using the procedures described in U.S. Pat. No. 5,661,017, herein incorporated by reference in its entirety.

The expressed protein or fragment thereof may be detected using methods known in the art that are specific for the particular protein or fragment. These detection methods may include the use of specific antibodies, formation of an enzyme product, or disappearance of an enzyme substrate. For example, if the protein or fragment thereof has enzymatic activity, an enzyme assay may be used. Alternatively, if polyclonal or monoclonal antibodies specific to the protein or fragment thereof are available, immunoassays may be employed using the antibodies to the protein or fragment thereof. The techniques of enzyme assay and immunoassay are well known to those skilled in the art.

The resulting protein or fragment thereof may be recovered by methods known in the arts. For example, the protein or fragment thereof may be recovered from the nutrient medium by conventional procedures including, but not limited to, centrifugation, filtration, extraction, spray-drying, evaporation, or precipitation. The recovered protein or fragment thereof may then be further purified by a variety of chromatographic procedures, e.g., ion exchange chromatography, gel filtration chromatography, affinity chromatography, or the like.

(f) Microbial Constructs and Transformed Microbial Cells

The nucleotide sequences of the present invention may be introduced into a wide variety of prokaryotic and eukaryotic microorganism hosts to express the C. sarokiniana proteins or fragments thereof of interest. The term “microorganism” includes prokaryotic and eukaryotic microbial species such as bacteria and fungi. Fungi include yeast and filamentous fungi. Illustrative prokaryotes, both Gram-negative and Gram-positive, include Enterobacteriaceae, such as Escherichia, Erwinia, Shigella, Salmonella, and Proteus; Bacillaceae; Rhizobiceae, such as Rhizobium; Spirillaceae, such as photobacterium, Zymomonas, Serratia, Aeromonas, Vibrio, Desulfovibrio, Spirillum; Lactobacillaceae; Pseudomonadaceae, such as Pseudomonas and Acetobacter; Azotobacteraceae, Actinomycetales, and Nitrobacteraceae. Among eukaryotes are fungi, such as Phycomycetes and Ascomycetes, which includes yeast, such as Saccharomyces and Schizosaccharomyces; and Basidiomycetes yeast, such as Rhodotorula, Aureobasidium, Sporobolomyces, and the like.

It is well known that exogenous nucleic acids encoding polypeptides of interest can be introduced into a microbial host cell, such as a bacterial cell or a fungal cell, using a recombinant construct. The present invention also relates to a fungal or bacterial recombinant construct comprising a structural nucleotide sequence encoding a C. sarokiniana protein or fragment thereof. The present invention also relates to a bacterial or fungal cell comprising a bacterial or fungal recombinant vector of the present invention. The present invention also relates to methods for obtaining a recombinant bacterial or fungal host cell, comprising introducing into a bacterial or fungal host cell an exogenous nucleic acid molecule of the present invention.

The bacterial recombinant vector may be a linear or a closed circular plasmid. The vector system may be a single vector or plasmid or two or more vectors or plasmids which together contain the total DNA to be introduced into the genome of the bacterial host. In addition, the bacterial vector may be an expression by vector. Nucleic acid molecules encoding C. sarokiniana proteins or fragments thereof can, for example, be suitably inserted into a replicable vector for expression in a bacterium under the control of a suitable promoter for that bacterium. Many vectors are available for this purpose, and selection of the appropriate vector will depend mainly on the size of the nucleic acid to be inserted into the vector and the particular host cell to be transformed with the vector. Each vector contains various components depending on its function (amplification of DNA or expression of DNA) and the particular host cell with which it is compatible. The vector components for bacterial transformation generally include, but are not limited to, one or more of the following: a signal sequence, an origin of replication, one or more selectable marker genes, and an inducible promoter allowing the expression of exogenous DNA.

In general, plasmid vectors containing replicon and control sequences that are derived from species compatible with the host cell are used in connection with bacterial hosts. The vector ordinarily carries a replication site, as well as marking sequences that are capable of providing phenotypic selection in transformed cells. For example, E. coli is typically transformed using pBR322, a plasmid derived from an E. coli species (see, e.g., Bolivar et al., Gene 2:95 (1977)). pBR322 contains genes for ampicillin and tetracycline resistance and thus provides easy means for identifying transformed cells. The pBR322 plasmid, or other microbial plasmid or phage, also generally contains, or is modified to contain, promoters that can be used by the microbial organism for expression of the selectable marker genes.

Nucleic acid molecules encoding C. sarokiniana proteins or fragments thereof may be expressed not only directly, but also as a fusion with another polypeptide, preferably a signal sequence or other polypeptide having a specific cleavage site at the N-terminus of the mature polypeptide. In general, the signal sequence may be a component of the vector, or it may be a part of the polypeptide encoding DNA that is inserted into the vector. The heterologous signal sequence selected should be one that is recognized and processed (i.e., cleaved by a signal peptidase) by the host cell. For bacterial host cells that do not recognize and process the native polypeptide signal sequence, the signal sequence is substituted by a bacterial signal sequence selected, for example, from the group consisting of the alkaline phosphatase, β-lactamase, , or heat-stable enterotoxin II leaders and the like.

Both expression and cloning vectors contain a nucleotide sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria.

Expression and cloning vectors also generally contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. One example of a selection scheme utilizes a drug to arrest growth of a host cell. Those cells that are successfully transformed with a heterologous protein or fragment thereof produce a protein conferring drug resistance and thus survive the selection regimen.

The expression vector for producing a polypeptide can also contains an inducible promoter that is recognized by the host bacterial organism and is operably linked to the nucleic acid encoding, for example, the nucleic acid molecule encoding the C. sarokiniana protein or fragment thereof of interest. Inducible promoters suitable for use with bacterial hosts include the β-lactamase, E. coli λ phage PL and PR, and E. coli galactose, arabinose, alkaline phosphatase, tryptophan (trp), and lactose operon promoter systems and variations thereof(Chang et al., Nature 275:615 (1978); Goeddel et al., Nature 281:544 (1979); Guzman et al., J. Bacteriol. 174:7716-7728 (1992); Goeddel, Nucleic Acids Res. 8:4057 (1980); EP 36,776) and hybrid promoters such as the tac promoter (deBoer et al., Proc. Natl. Acad. Sci. (USA) 80:21-25 (1983)). However, other known bacterial inducible promoters are suitable (Siebenlist et al., Cell 20:269 (1980)).

Promoters for use in bacterial systems also generally contain a Shine-Dalgarno (S.D.) sequence or a consensus sequence thereof operably linked to the DNA encoding the polypeptide of interest. The promoter can be removed from the bacterial source DNA by restriction enzyme digestion and inserted into the vector containing the desired DNA coding sequence, or vice versa.

Alternatively, the expression constructs can be integrated into the bacterial genome with an integrating vector. Integrating vectors typically contain at least one sequence homologous to the bacterial chromosome that allows the vector to integrate. Integrations appear to result from recombinations between homologous DNA in the vector and the bacterial chromosome. For example, integrating vectors constructed with DNA from various Bacillus strains integrate into the Bacillus chromosome (E.P.O. Pub. No. 127,328). Integrating vectors may also be comprised of bacteriophage or transposon sequences.

Construction of suitable vectors containing one or more of the above-listed components employs standard recombinant DNA techniques. Isolated plasmids or DNA fragments are cleaved, tailored, and re-ligated in the form desired to generate the plasmids required. Examples of available bacterial expression vectors include, but are not limited to, the multifunctional E. coli cloning and expression vectors such as Bluescript™ (Stratagene, La Jolla, Calif.), in which, for example, a C. sarokiniana protein or fragment thereof, may be ligated into the vector in frame with sequences for the amino-terminal Met and the subsequent 7 residues of β-galactosidase so that a hybrid protein is produced; pfN vectors (Van Heeke and Schuster J. Biol. Chem. 264:5503-5509 (1989)); and the like. pGEX vectors (Promega, Madison Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems are designed to include heparin, thrombin or factor XA protease cleavage sites so that the cloned polypeptide of interest can be released from the GST moiety at will.

It is, of course, necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. E. coli strain W3110 is a preferred host or parent host because it is a common host strain for recombinant DNA product fermentations. Preferably, the host cell should secrete minimal amounts of proteolytic enzymes.

Host cells are transfected and preferably transformed with the above-described vectors and cultured in conventional nutrient media modified as appropriate for inducing promoters, selecting transformants, or amplifying the genes encoding the desired sequences.

Numerous methods of transfection are known to the ordinarily skilled artisan, for example, calcium phosphate and electroporation. Depending on the host cell used, transformation is done using standard techniques appropriate to such cells. The calcium treatment employing calcium chloride, as described in section 1.82 of Sambrook et al., Molecular Cloning: A Laboratory Manual, New York: Cold Spring Harbor Laboratory Press, (1989), is generally used for bacterial cells that contain substantial cell-wall barriers. Another method for transformation employs polyethylene glycol/DMSO, as described in Chung and Miller (Chung and Miller, Nucleic Acids Res. 16:3580 (1988)). Yet another method is the use of the technique termed electroporation. In addition, bacterial cells can be readily transformed using various forms of phages (i.e., transducing, temperate, lytic and lysogenic), suicide vectors for inserting DNA directly into the chromosome, and through homologous recombination using either phages, suicide vectors or linear DNA.

Bacterial cells used to produce the polypeptide of interest for purposes of this invention are cultured in suitable media in which the promoters for the nucleic acid encoding the heterologous polypeptide can be artificially induced as described generally, e.g., in Sambrook et al., Molecular Cloning: A Laboratory Manual, New York: Cold Spring Harbor Laboratory Press, (1989). Examples of suitable media are given in U.S. Pat. Nos. 5,304,472 and 5,342,763.

A yeast recombinant construct can typically include one or more of the following: a promoter sequence, fusion partner sequence, leader sequence, transcription termination sequence, a selectable marker. These elements can be combined into an expression cassette, which may be maintained in a replicon, such as an extrachromosomal element (e.g., plasmids) capable of stable maintenance in a host, such as yeast or bacteria. The replicon may have two replication systems, thus allowing it to be maintained, for example, in yeast for expression and in a procaryotic host for cloning and amplification. Examples of such yeast-bacteria shuttle vectors include YEp24 (Botstein et al., Gene, 8:17-24 (1979)), pC1/1 (Brake et al., Proc. Natl. Acad. Sci USA, 81:4642-4646 (1984)), and YRpl7 (Stinchcomb et al., J. Mol. Biol., 158:157 (1982)). In addition, a replicon may be either a high or low copy number plasmid. A high copy number plasmid will generally have a copy number ranging from about 5 to about 200, and typically about 10 to about 150. A host containing a high copy number plasmid will preferably have at least about 10, and more preferably at least about 20.

Useful yeast promoter sequences can be derived from genes encoding enzymes in the metabolic pathway. Examples of such genes include alcohol dehydrogenase (ADH) (E.P.O. Pub. No. 284044), enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK) (E.P.O. Pub. No. 329203). The yeast PHO5 gene, encoding acid phosphatase, also provides useful promoter sequences (Myanohara et al., Proc. Natl. Acad. Sci. USA, 80:1 (1983)). In addition, synthetic promoters which do not occur in nature also function as yeast promoters. Examples of such hybrid promoters include the ADH regulatory sequence linked to the GAP transcription activation region (U.S. Pat. Nos. 4,876,197 and 4,880,734). Other examples of hybrid promoters include promoters which consist of the regulatory sequences of either the ADH2, GAL4, GAL10, or PHO5 genes, combined with the transcriptional activation region of a glycolytic enzyme gene such as GAP or PyK (E.P.O. Pub. No. 164556). Furthermore, a yeast promoter can include naturally occurring promoters of non-yeast origin that have the ability to bind yeast RNA polymerase and initiate transcription. Examples of such promoters include, inter alia, (Cohen et al., Proc. Natl. Acad. Sci. USA, 77:1078 (1980); Henikoffet al., Nature 283:835 (1981); Hollenberg et al., Curr.

Topics Microbiol. Immunol., 96:119 (1981); Mercerau-Puigalon et al., Gene, 11:163 (1980); and Panthier et al., Curr. Genet., 2:109 (1980)).

Intracellularly expressed fusion proteins provide an alternative to direct expression of the polypeptides of interest. Typically, a DNA sequence encoding the N-terminal portion of a stable protein, a fusion partner, is fused to the 5′ end of heterologous structural nucleotide sequence encoding the desired polypeptide. Upon expression, this construct will provide a fusion of the two amino acid sequences. The DNA sequence at the junction of the two amino acid sequences may or may not encode a cleavable site. See, e.g., E.P.O. Pub. No. 196056. Another example is a ubiquitin fusion protein. Such a ubiquitin fusion protein preferably retains a site for a processing enzyme (e.g. ubiquitin-specific processing protease) to cleave the ubiquitin from the polypeptide of the present invention. Through this method, therefore, a mature polypeptide can be isolated [see, P.C.T. WO 88/024066].

Alternatively, polypeptides or proteins can also be secreted from the cell into the growth media by creating chimeric DNA molecules that encode a fusion protein comprised of a leader sequence fragment that provides for secretion in yeast of the polypeptides. Preferably, there are processing sites encoded between the leader fragment and the polypeptide-encoding sequence fragment that can be cleaved either in vivo or in vitro. The leader sequence fragment typically encodes a signal peptide comprised of hydrophobic amino acids which direct the secretion of the protein from the cell.

DNA encoding suitable signal sequences can be derived from genes for secreted yeast proteins, such as the yeast invertase gene (E.P.O. Pub. No. 12873; J.P.O. Pub. No. 62,096,086) and the A-factor gene (U.S. Pat. No. 4,588,684). Alternatively, leaders of non-yeast origin, such as an interferon leader, exist that also provide for secretion in yeast (E.P.O. Pub. No. 60057).

A preferred class of secretion leaders are those that employ a fragment of the yeast alpha-factor gene, which contains both a “pre” signal sequence, and a “pro” region. The types of alpha-factor fragments that can be employed include the full-length pre-pro alpha factor leader (about 83 amino acid residues) as well as truncated alpha-factor leaders (typically about 25 to about 50 amino acid residues) (U.S. Pat. Nos. 4,546,083 and 4,870,008; and E.P.O. Pub. No. 324274). Additional leaders employing an alpha-factor leader fragment that provides for secretion include hybrid alpha-factor leaders made with a pre-sequence of a first yeast, but a pro-region from a second yeast alpha factor. See, e.g., P.C.T. WO 89/02463.

Examples of transcription terminator sequence and other yeast-recognized termination sequences, such as those coding for glycolytic enzymes, are known to those of skill in the art.

Alternatively, the expression constructs can be integrated into the yeast genome with an integrating vector. Integrating vectors typically contain at least one sequence homologous to a yeast chromosome that allows the vector to integrate, and preferably contain two homologous sequences flanking the expression construct. Integrations appear to result from recombinations between homologous DNA in the vector and the yeast chromosome (Orr-Weaver et al., Methods in Enzymol., 101:228-245 (1983)). An integrating vector may be directed to a specific locus in yeast by selecting the appropriate homologous sequence for inclusion in the vector. See Orr-Weaver et al., supra. One or more expression constructs may integrate, possibly affecting levels of recombinant protein produced (Rine et al., Proc. Natl. Acad. Sci. USA, 80:6750 (1983)). The chromosomal sequences included in the vector can occur either as a single segment in the vector, which results in the integration of the entire vector, or as two segments homologous to adjacent segments in the chromosome and flanking the expression construct in the vector, which results in the stable integration of only the expression construct.

Expression and transformation vectors, either extrachromosomal replicons or integrating vectors, have been developed for transformation into many yeasts. For example, expression vectors have been developed for, inter alia, the following yeasts: Candida albicans (Kurtz, et al., Mol. Cell. Biol., 6:142 (1986)), Candida maltosa (Kunze et al., J. Basic Microbiol., 25:141 (1985)); Hansenula polymorpha (Gleeson et al., J. Gen. Microbiol. 132:3459 (1986); Roggenkamp et al., Mol. Gen. Genet. 202:302 (1986)); Kluyveromyces fragilis (Das et al., J. Bacteriol. 158:1165 (1984)); Kluyveromyces lactis (De Louvencourt et al., J. Bacteriol. 154:737 (1983); Van den Berg et al., Bio/Technology 8:135 (1990)); Pichia guillerimondii (Kunze et al., J. Basic Microbi-ol. 25:141 (1985)); Pichia pastoris (Cregg et al., Mol. Cell. Biol. 5:3376 (1985); U.S. Pat. Nos. 4,837,148 and 4,929,555); Saccharomyces cerevisiae (Hinnen et al., Proc. Natl. Acad. Sci. USA 75:1929 (1978); Ito et al., J. Bacteriol. 153:163 (1983)); Schizosaccharomyces pombe (Beach and Nurse, Nature 300:706 (1981)); and Yarrowia lipolytica (Davidow, et al., Curr. Genet. 10:380471 (1985); and Gaillardin et al., Curr. Genet. 10:49 (1985)).

Methods of introducing exogenous nucleic acids into yeast hosts are well-known in the art, and typically include either the transformation of spheroplasts or of intact yeast cells treated with alkali cations. Transformation procedures usually vary with the yeast species to be transformed. See e.g., Kurtz et al., Mol. Cell. Biol. 6:142 (1986); Kunzeetal., J. Basic Microbiol. 25:141 (1985) for Candida. See, e.g., Gleeson et al., J. Gen. Microbiol. 132:3459 (1986); Roggenkamp et al., Mol. Gen. Genet. 202:302 (1986) for Hansenula. See, e.g., Das et al., J. Bacteriol. 158:1165 (1984); De Louvencourt et al., J. Bacteriol. 154:1165 (1983); Van den Berg et al., Bio/Technology 8:135 (1990) for Kluyveromyces. See, e.g., Cregg et al., Mol. Cell. Biol. 5:3376 (1985); Kunze et al., J. Basic Microbiol. 25:141 (1985); U.S. Pat. Nos. 4,837,148 and 4,929,555 for Pichia. See, e.g., Hinnen et al., Proc. Natl. Acad. Sci. USA 75:1929 (1978); Ito et al., J. Bacteriol. 153:163 (1983) for Saccharomyces. See, e.g., Beach and Nurse, Nature 300:706 (1981) for Schizosaccharomyces. See, e.g., Davidow et al., Curr. Genet. 10:39 (1985); Gaillardin et al., Curr. Genet. 10:49 (1985) for Yarrowia.

In order to obtain expression polypeptides or proteins of interest, recombinant microbial host cells derived from the transformants are incubated under conditions which allow expression of the recombinant polypeptide-encoding sequence. These conditions will vary, dependent upon the host cell selected. However, the conditions are readily ascertainable to those of ordinary skill and knowledge in the art.

Detection of polypeptides expressed in the transformed host cell may be performed by several methods. For example, a polypeptide or protein may be detected by its immunological reactivity with antibodies.

Polypeptides or proteins of the present invention may be isolated from the cell by lysis, if formed intracellularly, or isolated from the culture medium, if secreted, by conventional methods.

(g) Mammalian Constructs and Transformed Mammalian Cells

The present invention also relates to a mammalian recombinant expression vector comprising exogenous genetic material. The present invention also relates to a mammalian cell comprising a mammalian recombinant expression vector. The present invention also relates to methods for obtaining a recombinant mammalian host cell, comprising introducing into a mammalian cell exogenous genetic material.

The mammalian recombinant expression vector may be any vector which can be conveniently subjected to recombinant DNA procedures. Many vectors are available for this purpose, and a suitable expression vector is one that is compatible with the desired function (e.g., transient expression, long term expression, integration, replication, amplification) and in which the control elements are compatible with the host cell. The control elements are those non-translated regions of the vector -promoters, enhancers, 5′ and 3′ untranslated regions—which interact with host celluar proteins to carry out transcription and translation.

Vectors suitable for replication in mammalian cells may include viral replicons, or sequences that ensure integration of the sequence encoding C. sarokiniana protein homologues or fragments thereof into the host genome. Suitable vectors may include, for example, those derived from simian virus SV40, retroviruses, bovine papilloma virus, vaccinia virus, and adenovirus. The components of the vectors, e.g. replicons, selection genes, enhancers, promoters, and the like, may be obtained from natural sources or synthesized by known procedures. (See, Kaufman et al, J. Mol. Biol., 159:511-521 (1982); and Kaufman, Proc. Nati. Acad. Sci., USA, 82:689-693 (1985)).

A suitable vector may be one derived from vaccinia viruses. In this case, a nucleic acid molecule encoding a C. sarokiniana protein homologue or fragment thereof is inserted into the vaccinia genome. Techniques for the insertion of foreign DNA into the vaccinia virus genome are known in the art, and utilize, for example, homologous recombination. The insertion of the foreign DNA is generally into a gene which is non-essential in nature, for example, the thymidine kinase gene (tk), which also provides a selectable marker. Plasmid shuttle vectors that greatly facilitate the construction of recombinant viruses have been described (see, for example, Mackett et al, J. Virol. 49: 857 (1984); Chakrabarti et al., Mol. Cell. Biol. 5: 3403 (1985); Moss, In: Gene Transfer Vectors For Mammalian Cells (Miller and Calos, eds., Cold Spring Harbor Laboratory, N.Y., p. 10, (1987)). Expression of the C. sarokiniana protein homologues or fragments thereof then occurs in cells or animals which are infected with the live recombinant vaccinia virus.

Suitable mammalian expression vectors usually contain one or more eukaryotic control elements that are capable of expression in mammalian cells. The control element is comprised of at least a promoter to mediate transcription of foreign DNA sequences. Suitable promoters for mammalian cells are known in the art and include viral promoters such as that from simian virus 40 (SV40), cytomegalovirus (CMV), Rous sarcoma virus (RSV), adenovirus (ADV), and bovine papilloma virus (BPV).

In addition, the control element may also be comprised of a termination sequence and poly(A) addition sequences which are operably linked to nucleotide sequences encoding C. sarokiniana protein homologues or fragments thereof. The control element may also be comprised of an enhancer sequence which increases the expression of C. sarokiniana protein homologues or fragments thereof.

Furthermore, the control element may also be comprised of an enhancer, which is any regulatory DNA sequence that can stimulate transcription up to 1000-fold when linked to endogenous or heterologous promoters, with synthesis beginning at the normal mRNA start site. Enhancers are also active when they are placed upstream or downstream from the transcription initiation site, in either normal or flipped orientation, or at a distance of more than 1000 nucleotides from the promoter (Maniatis et al. Science, 236:1237 (1987); Alberts et al., Molecular Biology of the Cell, 2nd ed. (1989)). Enhancers derived from viruses may be particularly useful, because they typically have a broader host range. Examples include the SV40 early gene enhancer (Dijkema et al, EMBO J., 4:761 (1985)) and the enhancer/promoters derived from the long terminal repeat (LTR) of the Rous Sarcoma Virus (Gorman et al., Proc. Natl. Acad. Sci. 79:6777 (1982b)) and from human cytomegalovirus (Boshart et al., Cell, 41:521 (1985)). Additionally, some enhancers are regulatable and become active only in the presence of an inducer, such as a hormone or metal ion (Sassone-Corsi and Borelli, Trends Genet. 2:215 (1986); Maniatis et al. Science, 236:1237 (1987)).

Where selection is intended, sequences which encode selectable markers may also be included in the vector. Selectable markers for mammalian cells are known in the art, and include for example, thymidine kinase, dihydrofolate reductase (together with methotrexate as a DHFR amplifier), aminoglycoside phosphotransferase, hygromycin B phosphotransferase, asparagine synthetase, adenosine deaminase, metallothionien, and antibiotic resistant genes such as neomycin.

For homologous recombination, constructs can be prepared where the amplifiable gene will be flanked, normally on both sides with DNA homologous with the DNA of the target region. Depending upon the nature of the integrating DNA and the purpose of the integration, the homologous DNA will generally be within 100 kb, usually 50 kb, preferably about 25 kb, of the transcribed region of the target gene, more preferably within 2 kb of the target gene. Where modeling of the gene is intended, homology will usually be present proximal to the site of the mutation. By gene is intended the coding region and those sequences required for transcription of a mature mRNA. The homologous DNA may include the 5′-upstream region outside of the transcriptional regulatory region or comprising any enhancer sequences, transcriptional initiation sequences, adjacent sequences, or the like. The homologous region may include a portion of the coding region, where the coding region may be comprised only of an open reading frame or combination of no exons and introns. The homologous region may comprise all or a portion of an intron, where all or a portion of one or more exons may also be present. Alternatively, the homologous region may comprise the 3′-region, so as to comprise all or a portion of the transcriptional termination region, or the region 3′ of this region. The homologous regions may extend over all or a portion of the target gene or be outside the target gene comprising all or a portion of the transcriptional regulatory regions and/or the structural gene.

The integrating constructs may be prepared in accordance with conventional ways, where sequences may be synthesized, isolated from natural sources, manipulated, cloned, ligated, subjected to in vitro mutagenesis, primer repair, or the like. At various stages, the joined sequences may be cloned, and analyzed by restriction analysis, sequencing, or the like. Usually during the preparation of a construct where various fragments are joined, the fragments, intermediate constructs and constructs will be carried on a cloning vector comprising a replication system functional in a prokaryotic host, e.g., E. Coli, and a marker for selection, e.g., biocide resistance, complementation to an auxotrophic host, etc. Other functional sequences may also be present, such as polylinkers, for ease of introduction and excision of the construct or portions thereof, or the like. A large number of cloning vectors are available such as pBR322, the pUC series, etc. These constructs may then be used for integration into the primary mammalian host.

The mammalian cell expression vectors described herein may be synthesized by techniques well known to those skilled in this art. Other appropriate expression vectors of which numerous types are known in the art for mammalian expression can also be used for this purpose.

Mammalian cell lines available as hosts for expression are known in the art and include many immortalized cell lines available from the American Type Culture Collection (ATCC). Exemplary mammalian host cells include particularly primate cell lines and rodent cell lines, including transformed cell lines. Suitable cell lines include, but are not limited to, Chinese hamster ovary (CHO) cells, HeLa cells, baby hamster kidney (BHK) cells, monkey kidney cells (COS-1), human hepatocellular carcinoma cells (e.g., Hep G2), human adenovirus transformed 293 cells, mouse L-929 cells, HaK hamster cell lines, murine 3T3 cells derived from Swiss, Balb-c or NIH mice and a number of other cell lines.

The DNA can be introduced into the expression host by a variety of techniques that include calcium phosphate/DNA co-precipitates, microinjection of DNA into the nucleus, electroporation, yeast protoplast fusion with intact cells, transfection, polycations, e.g., polybrene, polyornithine, etc., or the like. The DNA may be single or double stranded DNA, linear or circular. The various techniques for transforming mammalian cells are well known (see Keown et al., Methods Enzymol. (1989), Keown et al., Methods Enzymol. 185:527-537 (1990); Mansour et al., Nature 336:348-352, (1988)).

(h) Insect Constructs and Transformed Insect Cells

The present invention also relates to an insect recombinant expression vectors comprising exogenous genetic material. The present invention also relates to an insect cell comprising an insect recombinant vector. The present invention also relates to methods for obtaining a recombinant insect host cell, comprising introducing into an insect cell exogenous genetic material.

The insect recombinant vector may be any vector which can be conveniently subjected to recombinant DNA procedures and can bring about the expression of the nucleic acid sequence. The choice of a vector will typically depend on the compatibility of the vector with the insect host cell into which the vector is to be introduced. The vector may be a linear or a closed circular plasmid. The vector system may be a single vector or plasmid or two or more vectors or plasmids which together contain the total DNA to be introduced into the genome of the insect host. In addition, the insect vector may be an expression vector. Nucleic acid molecules can be suitable inserted into a replication vector for expression in the insect cell under a suitable promoter for insect cells. Many vectors are available for this purpose, and selection of the appropriate vector will depend mainly on the size of the nucleic acid molecule to be inserted into the vector and the particular host cell to be transformed with the vector. Each vector contains various components depending on its function (amplification of DNA or expression of DNA) and the particular host cell with which it is compatible. The vector components for insect cell transformation generally include, but not limited to, one or more of the following: a signal sequence, and origin of replication, one or more marker genes, and an inducible promoter.

The insect vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the insect cell, is integrated into the genome and replicated together with the chromosome(s) into which it has been integrated. For integration, the vector may rely on the nucleic acid sequence of the vector for stable integration of the vector into the genome by homologous or nonhomologous recombination. Alternatively, the vector may contain additional nucleic acid sequences for directing integration by homologous recombination into the genome of the insect host. The additional nucleic acid sequences enable the vector to be integrated into the host cell genome at a precise location(s) in the chromosome(s). To increase the likelihood of integration at a precise location, there should be preferably two nucleic acid sequences which individually contain a sufficient number of nucleic acids, preferably 400 bp to 1500 bp, more preferably 800 bp to 1000 bp, which are highly homologous with the corresponding target sequence to enhance the probability of homologous recombination. These nucleic acid sequences may be any sequence that is homologous with a target sequence in the genome of the insect host cell, and, furthermore, may be non-encoding or encoding sequences.

Baculovirus expression vectors (BEVs) have become important tools for the expression of foreign genes, both for basic research and for the production of proteins with direct clinical applications in human and veterinary medicine (Doerfler, Curr. Top. Microbiol. Immunol. 131: 51-68 (1968); Luckow and Summers, Bio/Technology 6: 47-55 (1988a); Miller, Annual Review of Microbiol. 42: 177-199 (1988); Summers, Curr. Comm. Molecular Biology, Cold Spring Harbor Press, Cold Spring Harbor, N.Y. (1988); all of which are herein incorporated by reference in their entirety). BEVs are recombinant insect viruses in which the coding sequence for a chosen foreign gene has been inserted behind a baculovirus promoter in place of the viral gene, e.g., polyhedrin (Smith and Summers, U.S. Pat. No., 4,745,051, herein incorporated by reference in its entirety).

The use of baculovirus vectors relies upon the host cells being derived from Lepidopteran insects such as Spodoptera frugiperda or Trichoplusia ni. The preferred Spodoptera frugiperda cell line is the cell line Sf9. The Spodoptera frugiperda Sf9 cell line was obtained from American Type Culture Collection (Manassas, Va.) and is assigned accession number ATCC CRL 1711 (Summers and Smith, A Manual of Methods for Baculovirus Vectors and Insect Cell Culture Procedures, Texas Ag. Exper. Station Bulletin No. 1555 (1988), herein incorporated by reference in its entirety). Other insect cell systems, such as the silkworm B. mori may also be used.

The proteins expressed by the BEVs are, therefore, synthesized, modified and transported in host cells derived from Lepidopteran insects. Most of the genes that have been inserted and produced in the baculovirus expression vector system have been derived from vertebrate species. Other baculovirus genes in addition to the polyhedrin promoter may be employed to advantage in a baculovirus expression system. These include immediate-early (alpha), delayed-early (beta), late (gamma), or very late (delta), according to the phase of the viral infection during which they are expressed. The expression of these genes occurs sequentially, probably as the result of a “cascade” mechanism of transcriptional regulation. (Guarino and Summers, J. Virol. 57:563-571 (1986); Guarino and Summers, J. Virol. 61:2091-2099 (1987); Guarino and Summers, Virol. 162:444-451 (1988); all of which are herein incorporated by reference in their entirety).

Insect recombinant vectors are useful as an intermediates for the infection or transformation of insect cell systems. For example, an insect recombinant vector containing a nucleic acid molecule encoding a baculovirus transcriptional promoter followed downstream by an insect signal DNA sequence is capable of directing the secretion of the desired biologically active protein from the insect cell. The vector may utilize a baculovirus transcriptional promoter region derived from any of the over 500 baculoviruses generally infecting insects, such as for example the Orders Lepidoptera, Diptera, Orthoptera, Coleoptera and Hymenoptera, including for example but not limited to the viral DNAs of Autographa californica MNPV, Bombyx mori NPV, Trichoplusia ni MNPV, Rachiplusia ou MNPV or Galleria mellonella MNPV, wherein said baculovirus transcriptional promoter is a baculovirus immediate-early gene IE1 or IEN promoter; an immediate-early gene in combination with a baculovirus delayed-early gene promoter region selected from the group consisting of 39K and a HindIII-k fragment delayed-early gene; or a baculovirus late gene promoter. The immediate-early or delayed-early promoters can be enhanced with transcriptional enhancer elements. The insect signal DNA sequence may code for a signal peptide of a Lepidopteran adipokinetic hormone precursor or a signal peptide of the Manduca sexta adipokinetic hormone precursor (Summers, U.S. Pat. No. 5,155,037; herein incorporated by reference in its entirety). Other insect signal DNA sequences include a signal peptide of the Orthoptera Schistocerca gregaria locust adipokinetic hormone precurser and the Drosophila melanogaster cuticle genes CP1, CP2, CP3 or CP4 or for an insect signal peptide having substantially a similar chemical composition and function (Summers, U.S. Pat. No. 5,155,037).

Insect cells are distinctly different from animal cells. Insects have a unique life cycle and have distinct cellular properties such as the lack of intracellular plasminogen activators in insect cells which are present in vertebrate cells. Another difference is the high expression levels of protein products ranging from 1 to greater than 500 mg/liter and the ease at which cDNA can be cloned into cells (Frasier, In Vitro Cell. Dev. Biol. 25:225 (1989); Summers and Smith, In: A Manual of Methodsfor Baculovirus Vectors and Insect Cell Culture Procedures, Texas Ag. Exper. Station Bulletin No. 1555 (1988), both of which are incorporated by reference in their entirety).

Recombinant protein expression in insect cells is achieved by viral infection or stable transformation. For viral infection, the desired gene is cloned into baculovirus at the site of the wild-type polyhedron gene (Webb and Summers, Technique 2:173 (1990); Bishop and Posse, Adv. Gene Technol 1:55 (1990); both of which are incorporated by reference in their entirety). The polyhedron gene is a component of a protein coat in occlusions which encapsulate virus particles. Deletion or insertion in the polyhedron gene results the failure to form occlusion bodies. Occlusion negative viruses are morphologically different from occlusion positive viruses and enable one skilled in the art to identify and purify recombinant viruses.

The vectors of present invention preferably contain one or more selectable markers which permit easy selection of transformed cells. A-selectable marker is a gene the product of which provides, for example biocide or viral resistance, resistance to heavy metals, prototrophy to auxotrophs, and the like. Selection may be accomplished by co-transformation, e.g., as described in WO 91/17243, a nucleic acid sequence of the present invention may be operably linked to a suitable promoter sequence. The promoter sequence is a nucleic acid sequence which is recognized by the insect host cell for expression of the nucleic acid sequence. The promoter sequence contains transcription and translation control sequences which mediate the expression of the protein or fragment thereof. The promoter may be any nucleic acid sequence which shows transcriptional activity in the insect host cell of choice and may be obtained from genes encoding polypeptides either homologous or heterologous to the host cell.

For example, a nuclec acid molecule encoding a C. sarokiniana protein homologue or fragment thereof may also be operably linked to a suitable leader sequence. A leader sequence is a nontranslated region of a mRNA which is important for translation by the insect host. The leader sequence is operably linked to the 5′ terminus of the nucleic acid sequence encoding the protein or fragment thereof. The leader sequence may be native to the nucleic acid sequence encoding the protein or fragment thereof or may be obtained from foreign sources. Any leader sequence which is functional in the insect host cell of choice may be used in the present invention.

A polyadenylation sequence may also be operably linked to the 3′ terminus of the nucleic acid sequence of the present invention. The polyadenylation sequence is a sequence which when transcribed is recognized by the insect host to add polyadenosine residues to transcribed mRNA. The polyadenylation sequence may be native to the nucleic acid sequence encoding the protein or fragment thereof or may be obtained from foreign sources. Any polyadenylation sequence which is functional in the fungal host of choice may be used in the present invention.

To avoid the necessity of disrupting the cell to obtain the protein or fragment thereof, and to minimize the amount of possible degradation of the expressed polypeptide within the cell, it is preferred that expression of the polypeptide gene gives rise to a product secreted outside the cell. To this end, the protein or fragment thereof of the present invention may be linked to a signal peptide linked to the amino terminus of the protein or fragment thereof. A signal peptide is an amino acid sequence which permits the secretion of the protein or fragment thereof from the insect host into the culture medium. The signal peptide may be native to the protein or fragment thereof of the invention or may be obtained from foreign sources. The 5′ end of the coding sequence of the nucleic acid sequence of the present invention may inherently contain a signal peptide coding region naturally linked in translation reading frame with the segment of the coding region which encodes the secreted protein or fragment thereof.

At present, a mode of achieving secretion of a foreign gene product in insect cells is by way of the foreign gene's native signal peptide. Because the foreign genes are usually from non-insect organisms, their signal sequences may be poorly recognized by insect cells, and hence, levels of expression may be suboptimal. However, the efficiency of expression of foreign gene products seems to depend primarily on the characteristics of the foreign protein. On average, nuclear localized or non-structural proteins are most highly expressed, secreted proteins are intermediate, and integral membrane proteins are the least expressed. One factor generally affecting the efficiency of the production of foreign gene products in a heterologous host system is the presence of native signal sequences (also termed presequences, targeting signals, or leader sequences) associated with the foreign gene. The signal sequence is generally coded by a DNA sequence immediately following (5′ to 3′) the translation start site of the desired foreign gene.

The expression dependence on the type of signal sequence associated with a gene product can be represented by the following example: If a foreign gene is inserted at a site downstream from the translational start site of the baculovirus polyhedrin gene so as to produce a fusion protein (containing the N-terminus of the polyhedrin structural gene), the fused gene is highly expressed. But less expression is achieved when a foreign gene is inserted in a baculovirus expression vector immediately following the transcriptional start site and totally replacing the polyhedrin structural gene.

Insertions into the region −50 to −1 significantly alter (reduce) steady state transcription which, in turn, reduces translation of the foreign gene product. Use of the pVL941 vector optimizes transcription of foreign genes to the level of the polyhedrin gene transcription. Even though the transcription of a foreign gene may be optimal, optimal translation may vary because of several factors involving processing: signal peptide recognition, mRNA and ribosome binding, glycosylation, disulfide bond formation, sugar processing, oligomerization, for example.

The properties of the insect signal peptide are expected to be more optimal for the efficiency of the translation process in insect cells than those from vertebrate proteins. This phenomenon can generally be explained by the fact that proteins secreted from cells are synthesized as precursor molecules containing hydrophobic N-terminal signal peptides. The signal peptides direct transport of the select protein to its target membrane and are then cleaved by a peptidase on the membrane, such as the endoplasmic reticulum, when the protein passes through it.

Another exemplary insect signal sequence is the sequence encoding for Drosophila cuticle proteins such as CP1, CP2, CP3 or CP4 (Summers, U.S. Pat. No. 5,278,050; herein incorporated by reference in its entirety). Most of the 9 kb region of the Drosophila genome contains genes for the cuticle proteins has been sequenced. Four of the five cuticle genes contain a signal peptide coding sequence interrupted by a short intervening sequence (about 60 base pairs) at a conserved site. Conserved sequences occur in the 5′ mRNA untranslated region, in the adjacent 35 base pairs of upstream flanking sequence and at −200 base pairs from the mRNA start position in each of the cuticle genes.

Standard methods of insect cell culture, cotransfection and preparation of plasmids are set forth in Summers and Smith (Summers and Smith, A Manual of Methods for Baculovirus Vectors and Insect Cell Culture Procedures, Texas Agricultural Experiment Station Bulletin No. 1555, Texas A&M University (1987)). Procedures for the cultivation of viruses and cells are described in Volkman and Summers, J. Virol 19: 820-832 (1975) and Volkman et al., J. Virol 19: 820-832 (1976); both of which are herein incorporate by reference in their entirety.

(i) Computer Media

The nucleotide sequence provided in SEQ ID NO: 1, through SEQ ID NO:9395 or fragment thereof, or complement thereof, or a nucleotide sequence at least 90% identical, preferably 95%, identical even more preferably 99% or 100% identical to the sequence provided in SEQ ID NO: 1 through SEQ ID NO:9395 or fragment thereof, or complement thereof, can be “provided” in a variety of mediums to facilitate use. Such a medium can also provide a subset thereof in a form that allows a skilled artisan to examine the sequences.

In one application of this embodiment, a nucleotide sequence of the present invention can be recorded on computer readable media. As used herein, “computer readable media” refers to any medium that can be read and accessed directly by a computer. Such media include, but are not limited to: magnetic storage media, such as floppy discs, hard disc, storage medium, and magnetic tape: optical storage media such as CD-ROM; electrical storage media such as RAM and ROM; and hybrids of these categories such as magnetic/optical storage media. A skilled artisan can readily appreciate how any of the presently known computer readable mediums can be used to create a manufacture comprising computer readable medium having recorded thereon a nucleotide sequence of the present invention.

As used herein, “recorded” refers to a process for storing information on computer readable medium. A skilled artisan can readily adopt any of the presently known methods for recording information on computer readable medium to generate media comprising the nucleotide sequence information of the present invention. A variety of data storage structures are available to a skilled artisan for creating a computer readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of data processor structuring formats (e.g. text file or database) in order to obtain computer readable medium having recorded thereon the nucleotide sequence information of the present invention.

By providing one or more of nucleotide sequences of the present invention, a skilled artisan can routinely access the sequence information for a variety of purposes. Computer software is publicly available which allows a skilled artisan to access sequence information provided in a computer readable medium. The examples which follow demonstrate how software which implements the BLAST (Altschul et al., J. Mol. Biol. 215: 403-410 (1990), herein incorporated by reference in its entirety) and BLAZE (Brutlag, et al., Comp. Chem. 17: 203-207 (1993), herein incorporated by reference in its entirety) search algorithms on a Sybase system can be used to identify open reading frames (ORFs) within the genome that contain homology to ORFs or proteins from other organisms. Such ORFs are protein-encoding fragments within the sequences of the present invention and are useful in producing commercially important proteins such as enzymes used in amino acid biosynthesis, metabolism, transcription, translation, RNA processing, nucleic acid and a protein degradation, protein modification, and DNA replication, restriction, modification, recombination, and repair.

The present invention further provides systems, particularly computer-based systems, which contain the sequence information described herein. Such systems are designed to identify commercially important fragments of the nucleic acid molecule of the present invention. As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based system are suitable for use in the present invention.

As indicated above, the computer-based systems of the present invention comprise a data storage means having stored therein a nucleotide sequence of the present invention and the necessary hardware means and software means for supporting and implementing a search means. As used herein, “data storage means” refers to memory that can store nucleotide sequence information of the present invention, or a memory access means which can access manufactures having recorded thereon the nucleotide sequence information of the present invention. As used herein, “search means” refers to one or more programs which are implemented on the computer-based system to compare a target sequence or target structural motif with the sequence information stored within the data storage means. Search means are used to identify fragments or regions of the sequence of the present invention that match a particular target sequence or target motif. A variety of known algorithms are disclosed publicly and a variety of commercially available software for conducting search means are available can be used in the computer-based systems of the present invention. Examples of such software include, but are not limited to, MacPattern (EMBL), BLASTIN and BLASTIX (NCBIA). One of the available algorithms or implementing software packages for conducting homology searches can be adapted for use in the present computer-based systems.

The most preferred sequence length of a target sequence is from about 10 to 100 amino acids or from about 30 to 300 nucleotide residues. However, it is well recognized that during searches for commercially important fragments of the nucleic acid molecules of the present invention, such as sequence fragments involved in gene expression and protein processing, may be of shorter length.

As used herein, “a target structural motif,” or “target motif,” refers to any rationally selected sequence or combination of sequences in which the sequences the sequence(s) are chosen based on a three-dimensional configuration which is formed upon the folding of the target motif. There are a variety of target motifs known in the art. Protein target motifs include, but are not limited to, enzymatic active sites and signal sequences. Nucleic acid target motifs include, but are not limited to, promoter sequences, cis elements, hairpin structures and inducible expression elements (protein binding sequences).

Thus, the present invention further provides an input means for receiving a target sequence, a data storage means for storing the target sequences of the present invention sequence identified using a search means as described above, and an output means for outputting the identified homologous sequences. A variety of structural formats for the input and output means can be used to input and output information in the computer-based systems of the present invention. A preferred format for an output means ranks fragments of the sequence of the present invention by varying degrees of homology to the target sequence or target motif. Such presentation provides a skilled artisan with a ranking of sequences which contain various amounts of the target sequence or target motif and identifies the degree of homology contained in the identified fragment.

A variety of comparing means can be used to compare a target sequence or target motif with the data storage means to identify sequence fragments sequence of the present invention. For example, implementing software which implement the BLAST and BLAZE algorithms (Altschul et al., J. Mol. Biol. 215: 403-410 (1990), herein incorporated by reference in its entirety) can be used to identify open frames within the nucleic acid molecules of the present invention. A skilled artisan can readily recognize that any one of the publicly available homology search programs can be used as the search means for the computer-based systems of the present invention.

Uses of the Agents of the Present Invention

Nucleic acid molecules and fragments thereof of the present invention may be employed to obtain other nucleic acid molecules from the same species. Such nucleic acid molecules include the nucleic acid molecules that encode the complete coding sequence of a protein and promoters and flanking sequences of such molecules. In addition, such nucleic acid molecules include nucleic acid molecules that encode for other isozymes or gene family members. Such molecules can be readily obtained by using the above-described nucleic acid molecules or fragments thereof to screen cDNA or genomic libraries obtained from C. sarokiniana. Methods for forming such libraries are well known in the art.

Nucleic acid molecules and fragments thereof of the present invention may also be employed to obtain other nucleic acid molecules such as nucleic acid homologues. Such homologues include the nucleic acid molecules that encode, in whole or in part, protein homologues of other species, plants or other organisms. Such molecules can be readily obtained by using the above-described nucleic acid molecules or El-fragments thereof to screen cDNA or genomic libraries. Methods for forming such libraries are well known in the art. Such homologue molecules may differ in their nucleotide sequences from those found in one or more of SEQ ID NO: 1 through SEQ ID NO:9395 or complements thereof because complete complementarity is not needed for stable hybridization. The nucleic acid molecules of the present invention therefore also include molecules that, although capable of specifically hybridizing with the nucleic acid molecules may lack “complete complementarity.” In a particular embodiment, methods or 3′ or 5′RACE may be used to obtain such sequences (Frohman, M. A. et al., Proc. Natl. Acad. Sci. (U.S.A.) 85:8998-9002 (1988); Ohara, 0. et al., Proc. Natl. Acad. Sci. (U.S.A.) 86:5673-5677 (1989), both of which are herein incorporated by reference in their entirety).

Any of a variety of methods may be used to obtain one or more of the above-described nucleic acid molecules (Zamechik et al, Proc. Natl. Acad. Sci. (U.S.A.) 83: 4143-4146 (1986); Goodchild et al., Proc. Natl. Acad. Sci. (U.S.A.) 85: 5507-5511 (1988); Wickstrom et al., Proc. Nati. Acad. Sci. (U.S.A.) 85: 1028-1032 (1988),; Holt et al., Molec. Cell. Biol. 8: 963-973 (1988); Gerwirtz et al., Science 242: 1303-1306 (1988); Anfossi et al., Proc. Natl. Acad. Sci. (U.S.A.) 86: 3379-3383 (1989); Becker et al., EMBO J. 8: 36853691 (1989); all of which are herein incorporated by reference in their entirety). Automated nucleic acid synthesizers may be employed for this purpose. In lieu of such synthesis, the disclosed nucleic acid molecules may be used to define a pair of primers that can be used with the polymerase chain reaction (Mullis et al., Cold Spring Harbor Symp. Quant. Biol. 51: 263-273 (1986); Erlich et al., European Patent 50,424; European Patent 84,796, European Patent 258,017, European Patent 237,362; Mullis, European Patent 201,184; Mullis et al., U.S. Pat. No. 4,683,202; Erlich, U.S. Pat. No. 4,582,788; and Saiki, R. et al., U.S. Pat. No. 4,683,194, all of which are herein incorporated by reference in their entirety) to amplify and obtain any desired nucleic acid molecule or fragment.

Promoter sequence(s) and other genetic elements including but not limited to transcriptional regulatory elements associated with one or more of the disclosed nucleic acid sequences can also be obtained using the disclosed nucleic acid sequences provided herein. In one embodiment, such sequences are obtained by incubating EST nucleic acid molecules or preferably fragments thereof with members of genomic libraries and recovering clones that hybridize to the EST nucleic acid molecule or fragment thereof. In a second embodiment, methods of “chromosome walking,” or inverse PCR may be used to obtain such sequences (Frohman, et al., Proc. Natl. Acad. Sci. (U.S.A.) 85:8998-9002 (1988); Ohara, et al., Proc. Natl. Acad. Sci. (U.S.A.) 86: 5673-5677 (1989); Pang et al., Biotechniques, 22(6); 1046-1048 (1977); Huang et al., Methods Mol. Biol. 69: 89-96 (1977); Hartl et al., Methods Mol. Biol. 58: 293-301(1996), all of which are herein incorporated by reference in their entirety). In one embodiment, the disclosed ESTs are used to identify cDNAs whose analogous genes contain promoters with desirable expression patterns. Isolation and functional analysis of the 5′ flanking promoter sequences of these genes from genomic libraries, for example, using genomic screening methods and PCR techniques would result in the isolation of useful promoters and transcriptional regulatory elements. These methods are known to those of skill in the art and have been described (See for example Birren et al., Genome Analysis:Analyzing DNA, 1, (1997), Cold Spring Harbor Laboratory Press, Cold Spring Harbor, N.Y., herein incorporated by reference in its entirety). Promoters obtained utilizing the ESTs of the present invention could also be modified to affect their control characteristics. Examples of such modifications would include but are not limited to enhancer sequences as reported by Kay et al., Science 236:1299 (1987), herein incorporated by reference in its entirety.

In an aspect of the present invention, one or more of the agents of the present invention may be used to detecting the presence, absence or level of a organism, preferably a green alga and more preferably Chlorella, and even more preferably an C. sarokiniana in a sample. In another aspect of the present invention, one or more of the nucleic molecules of the present invention are used to determine the level (i.e., the concentration of mRNA in a sample, etc.) or pattern (i.e., the kinetics of expression, rate of decomposition, stability profile, etc.) of the expression encoded in part or whole by one or more of the nucleic acid molecule of the present invention (collectively, the “Expression Response” of a cell or tissue). As used herein, the Expression Response manifested by a cell or tissue is said to be “altered” if it differs from the Expression Response of cells or tissues of organisms not exhibiting the phenotype. To determine whether a Expression Response is altered, the Expression Response manifested by the cell or tissue of the organism exhibiting the phenotype is compared with that of a similar cell or tissue sample of a organism not exhibiting the phenotype. As will be appreciated, it is not necessary to re-determine the Expression Response of the cell or tissue sample of organisms not exhibiting the phenotype each time such a comparison is made; rather, the Expression Response of a particular organism may be compared with previously obtained values of normal organism. As used herein, the phenotype of the organism is any of one or more characteristics of an organism.

In one sub-aspect, such an analysis is conducted by determining the presence and/or identity of polymorphism(s) by one or more of the nucleic acid molecules of the present invention and more specifically, one or more of the EST nucleic acid molecule or fragment thereof which are associated with phenotype, or a predisposition to phenotype.

Any of a variety of molecules can be used to identify such polymorphism(s). In one embodiment, one or more of the EST nucleic acid molecules (or a sub-fragment thereof) may be employed as a marker nucleic acid molecule to identify such polymorphism(s). Alternatively, such polymorphisms can be detected through the use of a marker nucleic acid molecule or a marker protein that is genetically linked to (i.e., a polynucleotide that co-segregates with) such polymorphism(s).

In an alternative embodiment, such polymorphisms can be detected through the use of a marker nucleic acid molecule that is physically linked to such polymorphism(s). For this purpose, marker nucleic acid molecules comprising a nucleotide sequence of a polynucleotide located within 1 mb of the polymorphism(s), and more preferably within 100 kb of the polymorphism(s), and most preferably within 10 kb of the polymorphism(s) can be employed.

The genomes of animals and plants naturally undergo spontaneous mutation in the course of their continuing evolution (Gusella, Ann. Rev. Biochem. 55:831-854 (1986), herein incorporated by reference in its entirety). A “polymorphism” is a variation or difference in the sequence of the gene or its flanking regions that arises in some of the members of a species. The variant sequence and the “original” sequence co-exist in the species' population. In some instances, such co-existence is in stable or quasi-stable equilibrium.

A polymorphism is thus said to be “allelic,” in that, due to the existence of the polymorphism, some members of a species may have the original sequence (i.e., the original “allele”) whereas other members may have the variant sequence (i.e., the variant “allele”). In the simplest case, only one variant sequence may exist, and the polymorphism is thus said to be di-allelic. In other cases, the species' population may contain multiple alleles, and the polymorphism is termed tri-allelic, etc. A single gene may have multiple different unrelated polymorphisms. For example, it may have a di-allelic polymorphism at one site, and a multi-allelic polymorphism at another site.

The variation that defines the polymorphism may range from a single nucleotide variation to the insertion or deletion of extended regions within a gene. In some cases, the DNA sequence variations are in regions of the genome that are characterized by short tandem repeats (STRs) that include tandem di- or tri-nucleotide repeated motifs of nucleotides. Polymorphisms characterized by such tandem repeats are referred to as “variable number tandem repeat” (“VNTR”) polymorphisms. VNTRs have been used in identity analysis (Weber, U.S. Pat. No. 5,075,217; Armour, et al., FEBS Lett. 307:113-115 (1992); Jones, et al., Eur. J. Haematol. 39:144-147 (1987); Horn, et al., PCT Application WO91/14003; Jeffreys, European Patent Application 370,719; Jeffreys, U.S. Pat. No. 5,175,082; Jeffreys. et al., Amer. J. Hum. Genet. 39:11-24 (1986); Jeffreys. et al., Nature 316:76-79 (1985); Gray, et al., Proc. k Acad. Soc. Lond. 243:241-253 (1991); Moore, et al., Genomics 10:654-660 (1991); Jeffreys, et al., Anim. Genet. 18:1-15 (1987); Hillel, et al., Anim. Genet. 20:145-155 (1989); Hillel, et al., Genet. 124:783-789 (1990), all of which are herein incorporated by reference in their entirety).

The detection of polymorphic sites in a sample of DNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means.

The most preferred method of achieving such amplification employs the polymerase chain reaction (“PCR”) (Mullis, et al., Cold Spring Harbor Symp. Quant. Biol. 51:263-273 (1986); Erlich, et al., European Patent Appln. 50,424; European Patent Appln. 84,796, European Patent Application 258,017, European Patent Appln. 237,362; Mullis, European Patent Appln. 201,184; Mullis, etal., U.S. Pat. No. 4,683,202; Erlich., U.S. Pat. No. 4,582,788; and Saiki, et al., U.S. Pat. No. 4,683,194, all of which are herein incorporated by reference in their entirety), using primer pairs that are capable of hybridizing to the proximal sequences that define a polymorphism in its double-stranded form.

In lieu of PCR, alternative methods, such as the “Ligase Chain Reaction” (“LCR”) may be used (Barany, Proc. Natl. Acad. Sci. (U.S.A.) 88:189-193 (1991), herein incorporated by reference in its entirety). LCR uses two pairs of oligonucleotide probes to exponentially amplify a specific target. The sequences of each pair of oligonucleotides is selected to permit the pair to hybridize to abutting sequences of the same strand of the target. Such hybridization forms a substrate for a template-dependent ligase. As with PCR, the resulting products thus serve as a template in subsequent cycles and an exponential amplification of the desired sequence is obtained.

LCR can be performed with oligonucleotides having the proximal and distal sequences of the same strand of a polymorphic site. In one embodiment, either oligonucleotide will be designed to include the actual polymorphic site of the polymorphism. In such an embodiment, the reaction conditions are selected such that the oligonucleotides can be ligated together only if the target molecule either contains or lacks the specific nucleotide that is complementary to the polymorphic site present on the oligonucleotide. Alternatively, the oligonucleotides may be selected such that they do not include the polymorphic site (see, Segev, PCT Application WO 90/01069, herein incorporated by reference in its entirety).

The “Oligonucleotide Ligation Assay” (“OLA”) may alternatively be employed (Landegren, et al., Science 241:1077-1080 (1988), herein incorporated by reference in its entirety). The OLA protocol uses two oligonucleotides which are designed to be capable of hybridizing to abutting sequences of a single strand of a target. OLA, like LCR, is particularly suited for the detection of point mutations. Unlike LCR, however, OLA results in “linear” rather than exponential amplification of the target sequence.

Nickerson, et al. have described a nucleic acid detection assay that combines attributes of PCR and OLA (Nickerson, et al., Proc. Natl. Acad. Sci. (U.S.A.) 87:8923-8927 (1990), herein incorporated by reference in its entirety). In this method, PCR is used to achieve the exponential amplification of target DNA, which is then detected using OLA. In addition to requiring multiple, and separate, processing steps, one problem associated with such combinations is that they inherit all of the problems associated with PCR and OLA.

Schemes based on ligation of two (or more) oligonucleotides in the presence of nucleic acid having the sequence of the resulting “di-oligonucleotide”, thereby amplifying the di-oligonuclebtide, are also known (Wu et al., Genomics 4:560 (1989), herein incorporated by reference in its entirety), and may be readily adapted to the purposes of the present invention.

Other known nucleic acid amplification procedures, such as allele-specific oligomers, branched DNA technology, transcription-based amplification systems, or isothermal amplification methods may also be used to amplify and analyze such polymorphisms (Malek, et al., U.S. Pat. No. 5,130,238; Davey, et al., European Patent Application 329,822; Schuster et al., U.S. Pat. No. 5,169,766; Miller, et al., PCT Application WO 89/06700; Kwoh, et al., Proc. Natl. Acad. Sci. (U.S.A.) 86:1173-1177 (1989); Gingeras, et al., PCT Application WO 88/10315; Walker, et al., Proc. Natl. Acad. Sci. (U.S.A.) 89:392-396 (1992), all of which are herein incorporated by reference in their entirety).

The identification of a polymorphism can be determined in a variety of ways. By correlating the presence or absence of it in an plant with the presence or absence of a phenotype, it is possible to predict the phenotype of that plant. If a polymorphism creates or destroys a restriction endonuclease cleavage site, or if it results in the loss or insertion of DNA (e.g., a VNTR polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms” (“RFLPs”). RFLPs have been widely used in human and plant genetic analyses (Glassberg, UK Patent Application 2135774; Skolnick, et al., Cytogen. Cell Genet. 32:58-67 (1982); Botstein, etal., Ann. J. Hum. Genet. 32:314-331 (1980); Fischer, etal. PCT Application WO90/13668; Uhlen, PCT Application WO90/11369, all of which are herein incorporated by reference in their entirety).

Polymorphisms can also be identified by Single Strand Conformation Polymorphism (SSCP) analysis. The SSCP technique is a method capable of identifying most sequence variations in a single strand of DNA, typically between 150 and 250 nucleotides in length (Elles, Methods in Molecular Medicine: Molecular Diagnosis of Genetic Diseases, Humana Press (1996); Orita et al., Genomics 5: 874-879 (1989), both of which are herein incorporated by reference in their entirety). Under denaturing conditions a single strand of DNA will adopt a conformation that is uniquely dependent on its sequence conformation. This conformation usually will be different, even if only a single base is changed. Most conformations have been reported to alter the physical configuration or size sufficiently to be detectable by electrophoresis. A number of protocols have been described for SSCP including, but not limited to Lee et al., Anal. Biochem. 205: 289-293 (1992); Suzuki et al., Anal. Biochem. 192: 82-84 (1991); Lo et al., Nucleic Acids Research 20: 1005-1009 (1992); Sarkar et al., Genomics 13: 441-443 (1992), all of which are herein incorporated by reference in their entirety). It is understood that one or more of the nucleic acids of the present invention, may be utilized as markers or probes to detect polymorphisms by SSCP analysis.

Polymorphisms may also be found using a DNA fingerprinting technique called amplified fragment length polymorphism (AFLP), which is based on the selective PCR amplification of restriction fragments from a total digest of genomic DNA to profile that DNA (Vos, et al., Nucleic Acids Res. 23:4407-4414 (1995), herein incorporated by reference in its entirety). This method allows for the specific co-amplification of high numbers of restriction fragments, which can be visualized by PCR without knowledge of the nucleic acid sequence.

AFLP employs basically three steps. Initially, a sample of genomic DNA is cut with restriction enzymes and oligonucleotide adapters are ligated to the restriction fragments of the DNA. The restriction fragments are then amplified using PCR by using the adapter and restriction sequence as target sites for primer annealing. The selective amplification is achieved by the use of primers that extend into the restriction fragments, amplifying only those fragments in which the primer extensions match the nucleotide flanking the restriction sites. These amplified fragments are then visualized on a denaturing polyacrylamide gel.

AFLP analysis has been performed on Salix (Beismann, et al., Mol. Ecol. 6:989-993 (1997); Acinetobacter (Janssen, et al., Int. J. Syst. Bacteriol 47:1179-1187 (1997), both of which are herein incorporated by reference in their entirety), Aeromonas popoffi (Huys, et al., Int. J. Syst. Bacteriol. 47:1165-1171 (1997), herein incorporated by reference in its entirety), rice (McCouch, et al., Plant Mol. Biol. 35:89-99 (1997); Nandi, et al., Mol. Gen. Genet. 255:1-8 (1997); Cho, et al., Genome 39:373-378 (1996), all of which are herein incorporated by reference in their entirety), barley (Hordeum vulgare) (Simons, et al., Genomics 44:61-70 (1997); Waugh, et al., Mol. Gen. Genet. 255:311-321 (1997); Qi, et al., Mol. Genet. 254:330-336 (1997); Becker, et al., Mol. Gen. Genet. 249:65-73 (1995), all of which are herein incorporated by reference in their entirety), potato (Van der Voort, et al., Mol. Gen. Genet. 255:438-447 (1997); Meksem, et al., Mol. Gen. Genet. 249:74-81 (1995), both of which are herein incorporated by reference in their entirety), Phytophthora infestans (Van der Lee, et al., Fungal Genet. Biol. 21:278-291 (1997), herein incorporated by reference in its entirety), Bacillus anthracis (Keim, et al., J. Bacteriol. 179:818-824 (1997), herein incorporated by reference in its entirety), Astragalus cremnophylax (Travis, et al., Mol. Ecol. 5:735-745 (1996), herein incorporated by reference in its entirety), Arabidopsis (Cnops, et al., Mol. Gen. Genet. 253:32-41 (1996), herein incorporated by reference in its entirety), Escherichia coli (Lin, et al., Nucleic Acids Res. 24:3649-3650 (1996), herein incorporated by reference in its entirety), Aeromonas (Huys, et al., Int. J. Syst. Bacteriol. 46:572-580 (1996), herein incorporated by reference in its entirety), nematode (Folkertsma, et al., Mol. Plant Microbe Interact. 9:47-54 (1996), herein incorporated by reference in its entirety), tomato (Thomas, et al., Plant J. 8:785-794 (1995), herein incorporated by reference in its entirety), and human (Latorra, et al., PCR Methods Appl. 3:351-358 (1994), herein incorporated by reference in its entirety). AFLP analysis has also been used for fingerprinting mRNA (Money, et al., Nucleic Acids Res. 24:2616-2617 (1996); Bachem, et al., Plant J. 9:745-753 (1996), both of which are herein incorporated by 30 reference in their entirety). It is understood that one or more of the nucleic acid molecules of the present invention, may be utilized as markers or probes to detect polymorphisms by AFLP analysis for fingerprinting mRNA.

Polymorphisms may also be found using random amplified polymorphic DNA (RAPD) (Williams et al., Nucl. Acids Res. 18: 6531-6535 (1990), herein incorporated by reference in its entirety) and cleaveable amplified polymorphic sequences (CAPS) (Lyamichev et al., Science 260: 778-783 (1993), herein incorporated by reference in its entirety). It is understood that one or more of the nucleic acid molecules of the present invention, may be utilized as markers or probes to detect polymorphisms by RAPD or CAPS analysis.

Polymorphisms are useful, through linkage analysis, to define the genetic distances or physical distances between polymorphic traits. A physical map or ordered array of genomic DNA fragments in the desired region containing the gene may be used to characterize and isolate genes corresponding to desirable traits. For this purpose, yeast artificial chromosomes (YACs), bacterial artificial chromosomes (BACs), and cosmids are appropriate vectors for cloning large segments of DNA molecules. Although fewer clones are needed to make a contig for a specific genomic region by using YACs (Agyare et al., Genome Res. 7: 1-9 (1997); James et al., Genomics 32: 425-430 (1996), both of which are herein incorporated by reference in their entirety), chimerism in the inserted DNA fragment can arise. Cosmids are convenient for handling smaller-size DNA molecules and may be used for transformation in developing transgenic plants. BACs also carry DNA fragments and are less prone to chimerism.

Through genetic mapping, a fine scale linkage map can be developed using DNA markers, and, then, a genomic DNA library of large-sized fragments can be screened with molecular markers linked to the desired trait. Molecular markers are advantageous for agronomic traits that are otherwise difficult to tag, such as resistance to pathogens, insects and nematodes, tolerance to abiotic stresses, quality parameters and quantitative traits. The essential requirements for marker-assisted selection in a plant breeding program are: (1) the marker(s) should co-segregate or be closely linked with the desired trait; (2) an efficient means of screening large populations for the molecular marker(s) should be available; and (3) the screening technique should have high reproducibility across laboratories, be economical to use and be user-friendly. Molecular marker studies using near-isogenic lines (NILs) (Martin et al., Proc. Natl. Acad. Sci. (U.S.A). 88: 2336-2340 (1991); Young et al., Genetics 120: 579-585. (1988), both of which are herein incorporated by reference in their entirety), bulked segregant analysis (Michelmore et al., Proc. Natl. Acad. Sci. (U.S.A) 88: 9828-9832 (1991), herein incorporated by reference in its entirety) or recombinant inbred lines (Mohan et al., Theor. Appl. Genet. 87: 782-788 (1994), herein incorporated by reference in its entirety) have been used to map genes in different plant species (Coe and Neuffer, In: Genetic maps: locus maps of complex genomes, ed. S. J. O'Brien, Cold Spring Harbor Laboratory, Cold Spring Harbor, N.Y., 157-189 (1993), herein incorporated by reference in its entirety). It is understood that one or more of the nucleic acid molecules of the present invention may be used as molecular markers.

In accordance with this aspect of the present invention, a sample nucleic acid is obtained from cells. Any source of nucleic acid may be used. Preferably, the nucleic acid is genomic DNA. The nucleic acid is subjected to restriction endonuclease digestion. For example, one or more EST nucleic acid molecule or fragment thereof can be used as a probe in accordance with the above-described polymorphic methods. The polymorphism obtained in this approach can then be cloned to identify the mutation at the coding region which alters the protein's structure or regulatory region of the gene which affects its expression level.

In one aspect of the present invention, an evaluation can be conducted to determine whether a particular mRNA molecule is present. One or more of the nucleic acid molecules of the present invention, preferably one or more of the EST nucleic acid molecules of the present invention are utilized to detect the presence or quantity of the mRNA species. Such molecules are then incubated with cell or tissue extracts of a plant under conditions sufficient to permit nucleic acid hybridization. The detection of double-stranded probe-mRNA hybrid molecules is indicative of the presence of the mRNA; the amount of such hybrid formed is proportional to the amount of mRNA. Thus, such probes may be used to ascertain the level and extent of the mRNA production in a plant's cells or tissues. Such nucleic acid hybridization may be conducted under quantitative conditions (thereby providing a numerical value of the amount of the mRNA present). Alternatively, the assay may be conducted as a qualitative assay that indicates either that the mRNA is present, or that its level exceeds a user set, predefined value.

A principle of in situ hybridization is that a labeled, single-stranded nucleic acid probe will hybridize to a complementary strand of cellular DNA or RNA and, under the appropriate conditions, these molecules will form a stable hybrid. When nucleic acid hybridization is combined with histological techniques, specific DNA or RNA sequences can be identified within a single cell. An advantage of in situ hybridization over more conventional techniques for the detection of nucleic acids is that it allows an investigator to determine the precise spatial population (Angerer et al., Dev. Biol. 101: 477-484 (1984); Angerer et al., Dev. Biol. 112: 157-166 (1985); Dixon etal., EMBO J. 10: 1317-1324 (1991), all of which are herein incorporated by reference in their entirety). In situ hybridization may be used to measure the steady-state level of RNA accumulation. It is a sensitive technique and RNA sequences present in as few as 5-10 copies per cell can be detected (Hardin et al., J. Mol. Biol. 202: 417-431. (1989), herein incorporated by reference in its entirety). A number of protocols have been devised for in situ hybridization, each with tissue preparation, hybridization, and washing conditions (Meyerowitz, Plant Mol. Biol. Rep. 5: 242-250 (1987); Cox and Goldberg, In: Plant Molecular Biology: A Practical Approach (ed. C. H. Shaw), pp. 1-35. IRL Press, Oxford (1988); Raikhel et al., In situ RNA hybridization in plant tissues. In Plant Molecular Biology Manual, vol. B9: 1-32. Kluwer Academic Publisher, Dordrecht, Belgium (1989), all of which are herein incorporated by reference in their entirety).

In situ hybridization also allows for the localization of proteins within a tissue or cell (Wilkinson, In Situ Hybridization, Oxford University Press, Oxford (1992); Langdale, In Situ Hybridization 165-179 In: The Maize Handbook, eds. Freeling and Walbot, Springer-Verlag, New York (1994), both of which are herein incorporated by reference in their entirety). It is understood that one or more of the molecules of the present invention, preferably one or more of the EST nucleic acid molecules of the present invention or one or more of the antibodies of the present invention may be utilized to detect the expression level or pattern of a protein or mRNA thereof by in situ hybridization.

Fluorescent in situ hybridization also enables the localization of a particular DNA sequence along a chromosome which is useful, among other uses, for gene mapping, following chromosomes in hybrid lines or detecting chromosomes with translocations, transversions or deletions. In situ hybridization has been used to identify chromosomes in several plant species (Griffor et al., Plant Mol. Biol. 17: 101-109 (1991); Gustafson et al., Proc. Nat'l. Acad. Sci. (U.S.A). 87: 1899-1902 (1990); Mukai and Gill, Genome 34: 448-452. (1991); Schwarzacher and Heslop-Harrison, Genome 34: 317-323 (1991); Wang et al., Jpn. J. Genet. 66: 313-316 (1991); Parra and Windle, Nature Genetics, 5: 17-21 (1993), all of which are herein incorporated by reference in their entirety). It is understood that the nucleic acid molecules of the present L invention may be used as probes or markers to localize sequences along a chromosome.

It is also understood that one or more of the molecules of the present invention, preferably one or more of the EST nucleic acid molecules of the present invention or one or more of the antibodies of the present invention may be utilized to detect the expression level or pattern of a protein or mRNA thereof by in situ hybridization.

Further, it is also understood that any of the nucleic acid molecules of the present invention may be used as marker nucleic acids and or probes in connection with methods that require probes or marker nucleic acids. As used herein, a probe is an agent that is utilized to determine an attribute or feature (e.g. presence or absence, location, correlation, identity, etc.) or a molecule, cell, tissue or plant. As used herein, a marker nucleic acid is a nucleic acid molecule that is utilized to determine an attribute or feature (e.g., presence or absence, location, correlation, etc.) or a molecule, cell, tissue or plant.

Nucleic acid molecules of the present invention can be used to monitor expression. A microarray-based method for high-throughput monitoring of gene expression may be utilized to measure gene-specific hybridization targets. This ‘chip’-based approach involves using microarrays of nucleic acid molecules as gene-specific hybridization targets to quantitatively measure expression of the corresponding genes (Schena et al., Science 270: 467-470 (1995); Shalon, Ph.D. Thesis, Stanford University (1996), both of which are herein incorporated by reference in their entirety). Every nucleotide in a large sequence can be queried at the same time. Hybridization can be used to efficiently analyze nucleotide sequences.

Several microarray methods have been described. One method compares the sequences to be analyzed by hybridization to a set of oligonucleotides or cDNA molecules representing all possible subsequences (Bains and Smith, J. Theor. Biol. 135: 303 (1989), herein incorporated by reference in its entirety). A second method hybridizes the sample to an array of oligonucleotide or cDNA probes. An array consisting of oligonucleotides or cDNA molecules complementary to subsequences of a target sequence can be used to determine the identity of a target sequence, measure its amount, and detect differences between the target and a reference sequence. Nucleic acid molecules microarrays may also be screened with protein molecules or fragments thereof to determine nucleic acid molecules that specifically bind protein molecules or fragments thereof.

The microarray approach may also be used with polypeptide targets (U.S. Pat. Nos. 5,445,934; 5,143,854; 5,079,600; 4,923,901, all of which are herein incorporated by reference in their entirety). Essentially, polypeptides are synthesized on a substrate (microarray) and these polypeptides can be screened with either protein molecules or fragments thereof or nucleic acid molecules in order to screen for either protein molecules or fragments thereof or nucleic acid molecules that specifically bind the target polypeptides (Fodor et al., Science 251: 767-773 (1991), herein incorporated by reference in its entirety).

It is understood that one or more of the molecules of the present invention, preferably one or more of the nucleic acid molecules or protein molecules or fragments thereof of the present invention may be utilized in a microarray based method. In a preferred embodiment of the present invention, one or more of the C. sarokiniana nucleic acid molecules or protein molecules or fragments thereof of the present invention may be utilized in a microarray based method. A particular preferred microarray embodiment of the present invention is a microarray comprising nucleic acid molecules encoding genes or fragments thereof that are homologues of known genes or nucleic acid molecules that comprise genes or fragment thereof that elicit only limited or no matches to known genes. A further preferred microarray embodiment of the present invention is a microarray comprising nucleic acid molecules having genes or fragments thereof that are homologues of known genes and nucleic acid molecules that comprise genes or fragment thereof that elicit only limited or no matches to known genes.

Nucleic acid molecules of the present invention may be used in site directed mutagenesis. Site-directed mutagenesis may be utilized to modify nucleic acid sequences, particularly as it is a technique that allows one or more of the amino acids encoded by a nucleic acid molecule to be altered (e.g. a threonine to be replaced by a methionine). Three basic methods for site-directed mutagenesis are often employed. These are cassette mutagenesis (Wells et al., Gene 34: 315-23 (1985), herein incorporated by reference in its entirety), primer extension (Gilliam et al., Gene 12: 129-137 (1980); Zoller and Smith, Methods Enzymol, 100: 468-500 (1983); Dalbadie-McFarland et al., Proc. Natl. Acad. Sci. (U.S.A). 79: 6409-6413 (1982), all of which are herein incorporated by reference in their entirety) and methods based upon PCR (Scharf et al., Science 233: 1076-1078 (1986); Higuchi et al., Nucleic Acids Res. 16: 7351-7367 (1988), both of which are herein incorporated by reference in their entirety). Site-directed mutagenesis approaches are also described in EP 0 385 962, EP 0 359 472, and PCT Patent Application WO 93/07278, all of which are herein incorporated by reference by reference in their entirety.

Site-directed mutagenesis strategies have been applied to plants for both in vitro as well as in vivo site-directed mutagenesis (Lanz et al., J. Biol. Chem. 266: 9971-9976 (1991); Kovgan and Zhdanov, Biotekhnologiya 5: 148-154, No. 207160n, Chemical Abstracts 110: 225 (1989); Ge et al., Proc. Natl. Acad. Sci. (U.S.A.) 86: 4037-4041 (1989); Zhu et al., J. Biol. Chem. 271: 18494-18498 (1996); Chu et al., Biochemistry 33: 6150-6157 (1994), Small et al., EMBO J. 11: 1291-1296 (1992); Cho et al., Mol. Biotechnol. 8: 13-16 (1997); Kita et al., J. Biol. Chem. 271: 26529-26535 (1996); Jin et al., Mol. Microbiol 7: 555-562 (1993); Hatfield and Vierstra, J. Biol. Chem. 267: 14799-14803 (1992); Zhao et al., Biochemistry 31: 5093-5099 (1992), all of which are herein incorporated by reference in their entirety).

Any of the nucleic acid molecules of the present invention may either be modified by site-directed mutagenesis or used as, for example, nucleic acid molecules that are used to target other nucleic acid molecules for modification. It is understood that mutants with more than one altered nucleotide can be constructed using techniques that practitioners skilled in the art are familiar with such as isolating restriction fragments and ligating such fragments into an expression vector (see, for example, Sambrook et al., Molecular Cloning: A Laboratory Manual, Cold Spring Harbor Press (1989)). In a preferred embodiment of the present invention, one or more of the nucleic acid molecules or fragments thereof of the present invention may be modified by site-directed mutagenesis.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant organisms and the screening and isolating of clones, (see for example, Sambrook et al., Molecular Cloning: A Laboratory Manual, Cold Spring Harbor Press (1989); Mailga et al., Methods in Plant Molecular Biology, Cold Spring Harbor Press (1995); Birren et al., Genome Analysis: Analyzing DNA, 1, Cold Spring Harbor, N.Y., all of which are herein incorporated by reference in their entirety).

Having now generally described the invention, the same will be more readily understood through reference to the following examples which are provided by way of illustration, and are not intended to be limiting of the present invention, unless specified.

### EXAMPLE 1

The cDNA library (LIB3602) is prepared from the cell cultures of the eukaryotic unicellular green alga Chlorella sarokiniana. Chlorella cultures were grown under light in ammonium media (29 mM, NH₄Cl, 18.4 mM KH₂PO₄, 0.3 mM EDTA, 6.0 mM K₂SO₄, 0.34 mM CaCl₂·2 H ₂O, 1.5 mM MgCl·6 H₂O, 0.733 mM ZnCl₂, 0.189 mM CoCl₂·6H₂O, 0.351 mM CuCl₂·2 H₂O, 10.11 mM MnCl·4 H₂O, 0.190 mM NiCl₂·6 H₂O, 0.196 mM NH₄VO₃, 0.190 mM SnCl₂·2 H₂O, 38.82 mM H₃BO₃, 4.186 mM (NH₄)₆Mo₇O₂₄·4H₂O). The illuminated cultures were bubbled continuously with a CO₂-air mixture and kept in constant motion with a stir bar powered by a magnetic stirrer. Cells were harvested by centrifugation

Total RNA was purified from the harvested cell culture using Trizol reagent from Life Technologies (Gibco BRL, Life Technologies, Gaithersburg, Md. U.S.A.), essentially as recommended by the manufacturer. Poly A+ mRNA was purified by oligo dT chromatography (Oligotex kit by Qiagen).

Construction of cDNA libraries is well-known in the art and a number of cloning strategies exist. A number of cDNA library construction kits are commercially available. The Superscript™ Plasmid System for cDNA synthesis and Plasmid Cloning (Gibco BRL, Life Technologies, Gaithersburg, Md. U.S.A.) was used, following the conditions suggested by the manufacturer. The cDNA was ligated into the pSPORT 1 cloning vector.

### EXAMPLE 2

The cDNA library of the present invention, LIB3602, is plated on LB agar containing the appropriate antibiotics for selection and incubated at 37° C. for a sufficient time to allow the growth of individual colonies. Single colonies are individually placed in each well of 96-well microtiter plates containing LB liquid including the selective antibiotics. The plates are incubated overnight at approximately 37° C. with gentle shaking to promote growth of the cultures. The plasmid DNA is isolated from each clone using a commercially available kit such as Qiaprep plasmid isolation kits, using the conditions recommended by the manufacturer (Qiagen Inc., Santa Clarita, Calif.). Avariety of plasmid isolation kits are commercially available.

The template plasmid DNA clones are used for subsequent sequencing. For sequencing the cDNA library LIB3602, a commercially available sequencing kit, such as the ABI PRISM dRhodamine Terminator Cycle Sequencing Ready Reaction Kit with AmpliTaq® DNA Polymerase, FS, is used under the conditions recommended by the manufacturer (PE Applied Biosystems, Foster City, Calif.). The ESTs of the present invention are generated by sequencing initiated from the 5′ end of each cDNA clone.

Two basic methods can be used for DNA sequencing, the chain termination method of Sanger et al., Proc. Natl. Acad. Sci. (U.S.A.) 74: 5463-5467 (1977), herein incorporated by reference in its entirety and the chemical degradation method of Maxam and Gilbert, Proc. Natl. Acad. Sci. (U.S.A.) 74: 560-564 (1977), herein incorporated by reference in its entirety. Automation and advances in technology such as the replacement of radioisotopes with fluorescence-based sequencing have reduced the effort required to sequence DNA (Craxton, Method, 2: 20-26 (1991); Ju et al., Proc. Natl. Acad. Sci. (U.S.A.) 92: 4347-4351 (1995); Tabor and Richardson, Proc. Natl. Acad. Sci. (U.S.A.) 92: 6339-6343 (1995), all of which are herein incorporated by reference in their entirety). Automated sequencers are available from, for example, Pharmacia Biotech, Inc., Piscataway, N.J. (Pharmacia ALF), LI-COR, Inc., Lincoln, Nebr. (LI-COR 4,000) and Millipore, Bedford, Mass. (Millipore BaseStation).

In addition, advances in capillary gel electrophoresis have also reduced the effort required to sequence DNA and such advances provide a rapid high resolution approach for sequencing DNA samples (Swerdlow and Gesteland, Nucleic Acids Res. 18: 1415-1419(1990); Smith, Nature 349: 812-813 (1991); Luckey et al., Methods Enzymol. 218: 154-172 (1993); Lu et al., J. Chromatog. A. 680: 497-501 (1994); Carson etal., Anal. Chem. 65:3219-3226(1993); Huang etal., Anal. Chem. 64:2149-2154(1992); Kheterpal et al., Electrophoresis 17: 1852-1859 (1996); Quesada and Zhang, Electrophoresis 17: 1841-1851 (1996); Baba, Yakugaku Zasshi 117: 265-281 (1997), all of which are herein incorporated by reference in their entirety).

A number of sequencing techniques are known in the art, including fluorescence-based sequencing methodologies. These methods have the detection, automation and instrumentation capability necessary for the analysis of large volumes of sequence data. Currently, the 377 DNA Sequencer (Perkin-Elmer Corp., Applied Biosystems Div., Foster City, Calif.) allows the most rapid electrophoresis and data collection. With u these types of automated systems, fluorescent dye-labeled sequence reaction products are detected and data entered directly into the computer, producing a chromatogram that is subsequently viewed, stored, and analyzed using the corresponding software programs. These methods are known to those of skill in the art and have been described and reviewed (Birren et al., Genome Analysis: Analyzing DNA, 1, Cold Spring Harbor, N.Y., herein incorporated by reference in its entirety).

### EXAMPLE 3

This example illustrates sequence comparison to determine the similarity/identitiy of the test or query sequence with sequences in publicly available or proprietary databases. A characteristic feature of a protein or DNA sequence is that it can be compared with other known protein or DNA sequences. Sequence comparisons can be undertaken by determining the similarity of the test or query sequence with sequences in publicly available or proprietary databases (“similarity analysis”) or by searching for certain motifs (“intrinsic sequence analysis”)(e.g. cis elements)(Coulson, Trends in Biotechnology, 12: 76-80 (1994); Birren, et al., Genome Analysis, 1: 543-559 (1997); both of which are herein incorporated by reference in their entirety).

Similarity analysis includes database search and alignment. Examples of public databases include the DNA Database of Japan (DDBJ)(http://www.ddbj.nig.ac.jp/); GeneBank (http://www.ncbi.nlm.nih.gov/web/GenBank/Index.htlm); and the European Molecular Biology Laboratory Nucleotide sequence Database (EMBL) (http://www.ebi.ac.uk/ebi docs/embl db.html).

A number of different search algorithms have been developed, one example of which are the suite of programs referred to as BLAST programs. There are five implementations of BLAST, three designed for nucleotide sequences queries (BLASTN, BLASTX, and TBLASTX) and two designed for protein sequence queries (BLASTP and TBLASTN) (Coulson, Trends in Biotechnology, 12: 76-80 (1994); Birren, et al., Genome Analysis, 1: 543-559 (1997)).

BLASTN takes a nucleotide sequence (the query sequence) and its reverse complement and searches them against a nucleotide sequence database. BLASTN was designed for speed, not maximum sensitivity, and may not find distantly related coding sequences. BLASTX takes a nucleotide sequence, translates it in three forward reading frames and three reverse complement reading frames, and then compares the six translations against a protein sequence database. BLASTX is useful for sensitive analysis of preliminary (single-pass) sequence data and is tolerant of sequencing errors (Gish and States, Nature Genetics, 3: 266-272 (1993), herein incorporated by reference). BLASTN and BLASTX may be used in concert for analyzing EST data (Coulson, Trends in Biotechnology, 12: 76-80 (1994); Birren et al., Genome Analysis, 1: 543-559 (1997)).

Given a coding nucleotide sequence and the protein it encodes, it is often preferable to use the protein as the query sequence to search a database because of the greatly increased sensitivity to detect more subtle relationships. This is due to the larger alphabet of proteins (20 amino acids) compared with the alphabet of nucleotide sequences (4 bases), where it is far easier to obtain a match by chance. In addition, with nucleotide alignments, only a match (positive score) or a mismatch (negative score) is obtained, but with proteins, the presence of conservative amino acid substitutions can be taken into account. Here, a mismatch may yield a positive score if the non-identical residue has physicauchemical properties similar to the one it replaced. Various scoring matrices are used to supply the substitution scores of all possible amino acid pairs. A general purpose scoring system is the BLOSUM62 matrix (Henikoff and Henikoff, Proteins, 17: 49-61 (1993), herein incorporated by reference in its entirety), which is currently the default choice for BLAST programs. BLOSUM62 is tailored for alignments of moderately diverged sequences and thus may not yield the best results under all conditions. Altschul, J. Mol. Biol. 36: 290-300 (1993), herein incorporated by reference in its entirety, uses a combination of three matrices to cover all contingencies. This may improve sensitivity, but at the expense of slower searches. In practice, a single BLOSUM62 matrix is often used but others (PAM40 and PAM250) may be attempted when additional analysis is necessary. Low PAM matrices are directed at detecting very strong but localized sequence similarities, whereas high PAM matrices are directed at detecting long but weak alignments between very distantly related sequences.

Homologues in other organisms are available that can be used for comparative sequence analysis. Multiple alignments are performed to study similarities and differences in a group of related sequences. CLUSTAL W is a multiple sequence alignment package available that performs progressive multiple sequence alignments based on the method of Feng and Doolittle, J. Mol. Evol. 25: 351-360 (1987), the entirety of which is herein incorporated by reference. Each pair of sequences is aligned and the distance between each pair is calculated; from this distance matrix, a guide tree is calculated, and all of the sequences are progressively aligned based on this tree. A feature of the program is its sensitivity to the effect of gaps on the alignment; gap penalties are varied to encourage the insertion of gaps in probable loop regions instead of in the middle of structured regions. Users can specify gap penalties, choose between a number of scoring matrices, or supply their own scoring matrix for both the pairwise alignments and the multiple alignments. CLUSTAL W for UNIX and VMS systems is available at: ftp.ebi.ac.uk. Another program is MACAW (Schuler et al., Proteins, Struct. Func. Genet, 9:180-190 (1991), the entirety of which is herein incorporated by reference, for which both Macintosh and Microsoft Windows versions are available. MACAW uses a graphical interface, provides a choice of several alignment algorithms, and is available by anonymous ftp at: ncbi.nlm.nih.gov (directory/pub/macaw).

Sequence motifs are derived from multiple alignments and can be used to examine individual sequences or an entire database for subtle patterns. With motifs, it is sometimes possible to detect distant relationships that may not be demonstrable based on comparisons of primary sequences alone. Currently, the largest collection of sequence motifs in the world is PROSITE (Bairoch and Bucher, Nucleic Acid Research, 22: 3583-3589 (1994), the entirety of which is herein incorporated by reference.) PROSITE may be accessed via either the ExPASy server on the World Wide Web or anonymous ftp site. Many commercial sequence analysis packages also provide search programs that use PROSITE data.

A resource for searching protein motifs is the BLOCKS E-mail server developed by S. Henikoff, Trends Biochem Sci., 18:267-268 (1993); Henikoff and Henikoff, Nucleic Acid Research, 19:6565-6572 (1991); Henikoff and Henikoff, Proteins, 17: 49-61 (1993); all of which are herein incorporated by reference in their entirety). BLOCKS searches a protein or nucleotide sequence against a database of protein motifs or “blocks.” Blocks are defined as short, ungapped multiple alignments that represent highly conserved protein patterns. The blocks themselves are derived from entries in PROSITE as well as other sources. Either a protein or nucleotide query can be submitted to the BLOCKS server; if a nucleotide sequence is submitted, the sequence is translated in all six reading frames and motifs are sought in these conceptual translations. Once the search is completed, the server will return a ranked list of significant matches, along with an alignment of the query sequence to the matched BLOCKS entries.

Conserved protein domains can be represented by two-dimensional matrices, which measure either the frequency or probability of the occurrences of each amino acid residue and deletions or insertions in each position of the domain. This type of model, when used to search against protein databases, is sensitive and usually yields more accurate results than simple motif searches. Two popular implementations of this approach are profile searches (such as GCG program ProfileSearch) and Hidden Markov Models (HMMs)(Krough et al., J. Mol. Biol. 235:1501-1531 (1994); Eddy, Current Opinion in Structural Biology 6:361-365 (1996), both of which are herein incorporated by reference in their entirety). In both cases, a large number of common protein domains have been converted into profiles, as present in the PROSITE library, or HHM models, as in the Pfam protein domain library (Sonnhammer et al., Proteins 28:405-420 (1997), the entirety of which is herein incorporated by reference). Pfam contains more than 500 HMM models for enzymes, transcription factors, signal transduction molecules, and structural proteins. Protein databases can be queried with these profiles or HMM models, which will identify proteins containing the w domain of interest. For example, HMMSW or HMMFS, two programs in a public domain package called HMMER (Sonnhammer et al., Proteins 28:405-420 (1997)) can be used.

PROSITE and BLOCKS represent collected families of protein motifs. Thus, searching these -databases entails submitting a single sequence to determine whether or not that sequence is similar to the members of an established family. Programs working in the opposite direction compare a collection of sequences with individual entries in the protein databases. An example of such a program is the Motif Search Tool, or MoST (Tatusov et al. Proc. Natl. Acad. Sci. 91: 12091-12095 (1994), the entirety of which is herein incorporated by reference.) On the basis of an aligned set of input sequences, a weight matrix is calculated by using one of four methods (selected by the user); a weight matrix is simply a representation, position by position in an alignment, of how likely a particular amino acid will appear. The calculated weight matrix is then used to search the databases. To increase sensitivity, newly found sequences are added to the original data set, the weight matrix is recalculated, and the search is performed again. This procedure continues until no new sequences are found.

Table 1 lists the nucleic acid molecules encoding homologs of known proteins.

TABLE 1

| SEQ | | | | BLAST | | % | |
|---|---|---|---|---|---|---|---|
| ID No. | Clone ID | NCBI gi | Method | Score | E value | Ident | NCBI gi Description |
| 1 | LIB3602-077-Q6-K6-F1 | 2500354 | BLASTX | 866 | 3.00E−93 | 81 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 2 | LIB3602-006-Q1-K1-B6 | 2500354 | BLASTX | 862 | 8.00E−93 | 81 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 3 | LIB3602-076-Q6-K6-F1 | 2500354 | BLASTX | 844 | 1.00E−90 | 84 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 4 | LIB3602-092-Q6-K6-H1 | 2500354 | BLASTX | 844 | 1.00E−90 | 84 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 5 | LIB3602-066-Q1-K6-G9 | 2500354 | BLASTX | 840 | 3.00E−90 | 82 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 6 | LIB3602-074-Q1-K1-B8 | 2500354 | BLASTX | 828 | 8.00E−89 | 80 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 7 | LIB3602-018-Q6-K1-D3 | 2500354 | BLASTX | 807 | 2.00E−86 | 81 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 8 | LIB3602-049-Q6-K1-H3 | 2500354 | BLASTX | 769 | 6.00E−82 | 81 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 9 | LIB3602-093-Q6-K6-G1 | 2500354 | BLASTX | 758 | 1.00E−80 | 79 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 10 | LIB3602-080-Q6-K6-A8 | 2500354 | BLASTX | 758 | 2.00E−80 | 79 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 11 | LIB3602-090-Q6-K6-H7 | 2500354 | BLASTX | 757 | 2.00E−80 | 79 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 12 | LIB3602-028-Q6-K1-D12 | 2500354 | BLASTX | 715 | 1.00E−75 | 81 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 13 | LIB3602-091-Q6-K6-H7 | 2500354 | BLASTX | 595 | 2.00E−75 | 79 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 14 | LIB3602-094-Q6-K6-H7 | 2500354 | BLASTX | 583 | 2.00E−75 | 78 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 15 | LIB3602-058-Q6-K1-G2 | 2500354 | BLASTX | 694 | 3.00E−73 | 80 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 16 | LIB3602-106-Q1-K1-F7 | 2500354 | BLASTX | 666 | 3.00E−73 | 78 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 17 | LIB3602-093-Q6-K6-B12 | 2500354 | BLASTX | 666 | 2.00E−72 | 78 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 18 | LIB3602-046-Q6-K1-H6 | 2500354 | BLASTX | 660 | 3.00E−69 | 80 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 19 | LIB3602-018-Q6-K1-C3 | 2500354 | BLASTX | 620 | 1.00E−64 | 73 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 20 | LIB3602-095-Q6-K6-A11 | 2500354 | BLASTX | 586 | 1.00E−64 | 78 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 21 | LIB3602-057-Q6-K1-F8 | 2500354 | BLASTX | 609 | 3.00E−63 | 78 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 22 | LIB3602-076-Q6-K6-A12 | 2500354 | BLASTX | 584 | 3.00E−60 | 80 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 23 | LIB3602-034-Q6-K1-F12 | 2500354 | BLASTX | 581 | 4.00E−60 | 80 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 24 | LIB3602-094-Q6-K6-A11 | 2500354 | BLASTX | 572 | 8.00E−59 | 76 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 25 | LIB3602-032-Q6-K1-A5 | 2500354 | BLASTX | 523 | 3.00E−53 | 78 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 26 | LIB3602-058-Q6-K1-E12 | 2500354 | BLASTX | 502 | 9.00E−51 | 77 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 27 | LIB3602-035-Q1-K1-F12 | 2500354 | BLASTX | 494 | 9.00E−50 | 77 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 28 | LIB3602-038-Q6-K1-B9 | 2500354 | BLASTX | 481 | 2.00E−48 | 76 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 29 | LIB3602-056-Q6-K1-D4 | 2500354 | BLASTX | 466 | 1.00E−46 | 76 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 30 | LIB3602-070-Q1-K1-E9 | 2500354 | BLASTX | 462 | 6.00E−46 | 72 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 31 | LIB3602-023-Q6-K1-H11 | 2500354 | BLASTX | 440 | 1.00E−43 | 75 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 32 | LIB3602-095-Q6-K6-D8 | 2500354 | BLASTX | 419 | 4.00E−41 | 74 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 33 | LIB3602-002-P1-K6-E9 | 2500354 | BLASTX | 419 | 6.00E−41 | 74 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 34 | LIB3602-079-Q6-K6-E1 | 2500354 | BLASTX | 390 | 1.00E−37 | 66 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 35 | LIB3602-055-Q6-K1-A8 | 2500354 | BLASTX | 367 | 5.00E−35 | 71 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 36 | LIB3602-032-Q6-K1-B3 | 2500354 | BLASTX | 335 | 3.00E−31 | 70 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 37 | LIB3602-106-Q1-K1-D7 | 2500354 | BLASTX | 186 | 4.00E−29 | 66 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 38 | LIB3602-018-Q6-K1-B7 | 2500354 | BLASTX | 311 | 1.00E−28 | 69 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 39 | LIB3602-093-Q6-K6-E6 | 2500354 | BLASTX | 301 | 2.00E−27 | 69 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 40 | LIB3602-008-Q6-K1-G11 | 2500354 | BLASTX | 274 | 3.00E−24 | 68 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 41 | LIB3602-018-Q6-K1-B1 | 2500354 | BLASTX | 202 | 1.00E−15 | 50 | 60S RIBOSOMAL PROTEIN L10 (EQM) |
| | | | | | | | >gi\|1902894\|dbj\|BAA19462.1\| (AB001891) QM |
| | | | | | | | family protein [Solanum melongena] |
| 42 | LIB3602-028-Q6-K1-H6 | 2851508 | BLASTX | 541 | 3.00E−55 | 62 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 43 | LIB3602-101-Q1-K1-B12 | 2851508 | BLASTX | 541 | 3.00E−55 | 62 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 44 | LIB3602-062-Q6-K1-G12 | 2851508 | BLASTX | 541 | 3.00E−55 | 62 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 45 | LIB3602-009-Q6-K1-D1 | 2851508 | BLASTX | 541 | 3.00E−55 | 62 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 46 | LIB3602-078-Q6-K6-G1 | 2851508 | BLASTX | 541 | 4.00E−55 | 62 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 47 | LIB3602-073-Q1-K1-E10 | 2851508 | BLASTX | 536 | 1.00E−54 | 61 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 48 | LIB3602-067-Q1-K1-G5 | 2851508 | BLASTX | 536 | 1.00E−54 | 61 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 49 | LIB3602-072-Q1-K1-D10 | 2851508 | BLASTX | 536 | 1.00E−54 | 61 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 50 | LIB3602-036-Q6-K1-H11 | 2851508 | BLASTX | 536 | 1.00E−54 | 61 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 51 | LIB3602-112-Q1-K1-B2 | 2851508 | BLASTX | 536 | 1.00E−54 | 61 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 52 | LIB3602-092-Q6-K6-C1 | 2851508 | BLASTX | 531 | 5.00E−54 | 61 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 53 | LIB3602-058-Q6-K1-E10 | 2851508 | BLASTX | 521 | 6.00E−53 | 62 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 54 | LIB3602-009-Q6-K1-B10 | 2851508 | BLASTX | 497 | 4.00E−50 | 65 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 55 | LIB3602-020-Q6-K1-B7 | 2851508 | BLASTX | 495 | 6.00E−50 | 60 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 56 | LIB3602-102-Q1-K1-B7 | 2851508 | BLASTX | 494 | 8.00E−50 | 66 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 57 | LIB3602-070-Q1-K1-F1 | 2851508 | BLASTX | 490 | 3.00E−49 | 60 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 58 | LIB3602-056-Q6-K1-B2 | 2851508 | BLASTX | 485 | 9.00E−49 | 65 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 59 | LIB3602-040-Q6-K1-G5 | 2851508 | BLASTX | 468 | 9.00E−47 | 60 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 60 | LIB3602-116-Q1-K1-G12 | 2851508 | BLASTX | 468 | 1.00E−46 | 60 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 61 | LIB3602-108-Q1-K1-B3 | 2851508 | BLASTX | 364 | 7.00E−45 | 65 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 62 | LIB3602-051-Q6-K1-E3 | 2851508 | BLASTX | 443 | 9.00E−44 | 58 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 63 | LIB3602-020-Q6-K1-A2 | 2851508 | BLASTX | 435 | 5.00E−43 | 64 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 64 | LIB3602-095-Q6-K6-G12 | 2851508 | BLASTX | 429 | 4.00E−42 | 58 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 65 | LIB3602-044-Q6-K1-C10 | 2851508 | BLASTX | 418 | 4.00E−41 | 67 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 66 | LIB3602-004-Q1-K1-G2 | 2851508 | BLASTX | 419 | 5.00E−41 | 60 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 67 | LIB3602-082-Q6-K6-G1 | 2851508 | BLASTX | 416 | 1.00E−40 | 59 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 68 | LIB3602-109-Q1-K1-D10 | 2851508 | BLASTX | 411 | 4.00E−40 | 60 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 69 | LIB3602-049-Q6-K1-D8 | 2851508 | BLASTX | 394 | 2.00E−38 | 71 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 70 | LIB3602-015-Q6-K1-E7 | 2851508 | BLASTX | 391 | 9.00E−38 | 59 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 71 | LIB3602-093-Q6-K6-C9 | 2851508 | BLASTX | 374 | 1.00E−35 | 52 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 72 | LIB3602-021-Q6-K1-H6 | 2851508 | BLASTX | 347 | 5.00E−33 | 71 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 73 | LIB3602-102-Q1-K1-C1 | 2851508 | BLASTX | 327 | 3.00E−30 | 56 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 74 | LIB3602-102-Q1-K1-D2 | 2851508 | BLASTX | 261 | 1.00E−22 | 55 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 75 | LIB3602-059-Q6-K1-A8 | 2851508 | BLASTX | 255 | 7.00E−22 | 54 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 76 | LIB3602-040-Q6-K1-G9 | 2851508 | BLASTX | 246 | 6.00E−21 | 53 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 77 | LIB3602-109-Q1-K1-H11 | 2851508 | BLASTX | 215 | 1.00E−17 | 57 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 78 | LIB3602-009-Q6-K1-D2 | 2851508 | BLASTX | 200 | 1.00E−15 | 62 | 60S RIBOSOMAL PROTEIN L21 |
| | | | | | | | >gi\|2160162\|gb\|AAB60725.1\| (AC000132) Similar |
| | | | | | | | to ribosomal protein L21 (gb\|L38826). ESTs |
| | | | | | | | gb\|AA395597, gb\|ATTS5197 come from this gene. |
| | | | | | | | [Arabidopsis thaliana] |
| | | | | | | | >gi\|3482935\|gb\|AAC33220.1\| (AC003970) Putative |
| | | | | | | | ribosomal protein L21 [Ar |
| 79 | LIB3602-080-Q6-K6-H6 | 4139216 | BLASTX | 800 | 1.00E−85 | 79 | (AF104630) light harvesting complex II protein |
| | | | | | | | precursor [Chlamydomonas reinhardtii] |
| 80 | LIB3602-088-Q6-K6-C6 | 4139216 | BLASTX | 732 | 1.00E−77 | 75 | (AF104630) light harvesting complex II protein |
| | | | | | | | precursor [Chlamydomonas reinhardtii] |
| 81 | LIB3602-090-Q6-K6-H2 | 4139216 | BLASTX | 713 | 2.00E−75 | 75 | (AF104630) light harvesting complex II protein |
| | | | | | | | precursor [Chlamydomonas reinhardtii] |
| 82 | LIB3602-071-Q1-K1-C6 | 4139216 | BLASTX | 707 | 1.00E−74 | 74 | (AF104630) light harvesting complex II protein |
| | | | | | | | precursor [Chlamydomonas reinhardtii] |
| 83 | LIB3602-027-Q6-K1-E8 | 4139216 | BLASTX | 694 | 3.00E−73 | 74 | (AF104630) light harvesting complex II protein |
| | | | | | | | precursor [Chlamydomonas reinhardtii] |
| 84 | LIB3602-074-Q1-K1-F10 | 4139216 | BLASTX | 689 | 2.00E−72 | 75 | (AF104630) light harvesting complex II protein |
| | | | | | | | precursor [Chlamydomonas reinhardtii] |
| 85 | LIB3602-028-Q6-K1-F7 | 4139216 | BLASTX | 688 | 2.00E−72 | 75 | (AF104630) light harvesting complex II protein |
| | | | | | | | precursor [Chlamydomonas reinhardtii] |
| 86 | LIB3602-106-Q1-K1-E11 | 4139216 | BLASTX | 686 | 3.00E−72 | 74 | (AF104630) light harvesting complex II protein |
| | | | | | | | precursor [Chlamydomonas reinhardtii] |
| 87 | LIB3602-113-Q1-K1-A8 | 4139216 | BLASTX | 668 | 5.00E−70 | 72 | (AF104630) light harvesting complex II protein |
| | | | | | | | precursor [Chlamydomonas reinhardtii] |
| 88 | LIB3602-063-Q1-K6-B5 | 4139216 | BLASTX | 656 | 1.00E−68 | 74 | (AF104630) light harvesting complex II protein |
| | | | | | | | precursor [Chlamydomonas reinhardtii] |
| 89 | LIB3602-092-Q6-K6-A8 | 4139216 | BLASTX | 643 | 4.00E−67 | 72 | (AF104630) light harvesting complex II protein |
| | | | | | | | precursor [Chlamydomonas reinhardtii] |
| 90 | LIB3602-118-Q1-K1-H5 | 4139216 | BLASTX | 479 | 4.00E−65 | 77 | (AF104630) light harvesting complex II protein |
| | | | | | | | precursor [Chlamydomonas reinhardtii] |
| 91 | LIB3602-103-Q1-K1-E7 | 4139216 | BLASTX | 611 | 2.00E−63 | 77 | (AF104630) light harvesting complex II protein |
| | | | | | | | precursor [Chlamydomonas reinhardtii] |
| 92 | LIB3602-046-Q6-K1-B10 | 4139216 | BLASTX | 605 | 8.00E−63 | 78 | (AF104630) light harvesting complex II protein |
| | | | | | | | precursor [Chlamydomonas reinhardtii] |
| 93 | LIB3602-040-Q6-K1-H5 | 4139216 | BLASTX | 603 | 2.00E−62 | 78 | (AF104630) light harvesting complex II protein |
| | | | | | | | precursor [Chlamydomonas reinhardtii] |
| 94 | LIB3602-015-Q6-K1-D11 | 4139216 | BLASTX | 583 | 4.00E−60 | 80 | (AF104630) light harvesting complex II protein |
| | | | | | | | precursor [Chlamydomonas reinhardtii] |
| 95 | LIB3602-035-Q1-K1-D5 | 4139216 | BLASTX | 580 | 8.00E−60 | 80 | (AF104630) light harvesting complex II protein |
| | | | | | | | precursor [Chlamydomonas reinhardtii] |
| 96 | LIB3602-063-Q1-K6-F12 | 4139216 | BLASTX | 569 | 2.00E−58 | 68 | (AF104630) light harvesting complex II protein |
| | | | | | | | precursor [Chlamydomonas reinhardtii] |
| 97 | LIB3602-088-Q6-K6-A2 | 4139216 | BLASTX | 561 | 1.00E−57 | 64 | (AF104630) light harvesting complex II protein |
| | | | | | | | precursor [Chlamydomonas reinhardtii] |
| 98 | LIB3602-111-Q1-K1-D10 | 4139216 | BLASTX | 437 | 4.00E−57 | 68 | (AF104630) light harvesting complex II protein |
| | | | | | | | precursor [Chlamydomonas reinhardtii] |
| 99 | LIB3602-094-Q6-K6-D5 | 4139216 | BLASTX | 544 | 2.00E−55 | 71 | (AF104630) light harvesting complex II protein |
| | | | | | | | precursor [Chlamydomonas reinhardtii] |
| 100 | LIB3602-011-Q6-K1-H9 | 4139216 | BLASTX | 538 | 7.00E−55 | 71 | (AF104630) light harvesting complex II protein |
| | | | | | | | precursor [Chlamydomonas reinhardtii] |
| 101 | LIB3602-073-Q1-K1-B7 | 4139216 | BLASTX | 538 | 8.00E−55 | 71 | (AF104630) light harvesting complex II protein |
| | | | | | | | precursor [Chlamydomonas reinhardtii] |
| 102 | LIB3602-037-Q6-K1-G7 | 4139216 | BLASTX | 530 | 4.00E−54 | 74 | (AF104630) light harvesting complex II protein |
| | | | | | | | precursor [Chlamydomonas reinhardtii] |
| 103 | LIB3602-113-Q1-K1-E12 | 4139216 | BLASTX | 531 | 5.00E−54 | 71 | (AF104630) light harvesting complex II protein |
| | | | | | | | precursor [Chlamydomonas reinhardtii] |
| 104 | LIB3602-071-Q1-K1-B11 | 4139216 | BLASTX | 517 | 2.00E−52 | 74 | (AF104630) light harvesting complex II protein |
| | | | | | | | precursor [Chlamydomonas reinhardtii] |
| 105 | LIB3602-026-Q6-K1-E12 | 4139216 | BLASTX | 503 | 9.00E−51 | 71 | (AF104630) light harvesting complex II protein |
| | | | | | | | precursor [Chlamydomonas reinhardtii] |
| 106 | LIB3602-085-Q6-K1-F8 | 4139216 | BLASTX | 499 | 3.00E−50 | 71 | (AF104630) light harvesting complex II protein |
| | | | | | | | precursor [Chlamydomonas reinhardtii] |
| 107 | LIB3602-034-Q6-K1-D5 | 4139216 | BLASTX | 469 | 4.00E−47 | 85 | (AF104630) light harvesting complex II protein |
| | | | | | | | precursor [Chlamydomonas reinhardtii] |
| 108 | LIB3602-120-Q1-K1-E1 | 4139216 | BLASTX | 450 | 2.00E−44 | 70 | (AF104630) light harvesting complex II protein |
| | | | | | | | precursor [Chlamydomonas reinhardtii] |
| 109 | LIB3602-108-Q1-K1-D3 | 4139216 | BLASTX | 446 | 3.00E−44 | 62 | (AF104630) light harvesting complex II protein |
| | | | | | | | precursor [Chlamydomonas reinhardtii] |
| 110 | LIB3602-117-Q1-K1-D10 | 4139216 | BLASTX | 215 | 1.00E−21 | 73 | (AF104630) light harvesting complex II protein |
| | | | | | | | precursor [Chlamydomonas reinhardtii] |
| 111 | LIB3602-043-Q6-K1-G6 | 548774 | BLASTX | 665 | 1.00E−69 | 59 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 112 | LIB3602-074-Q1-K1-G11 | 548774 | BLASTX | 634 | 5.00E−66 | 59 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 113 | LIB3602-041-Q6-K1-D5 | 548774 | BLASTX | 631 | 9.00E−66 | 62 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 114 | LIB3602-086-Q6-K1-E6 | 548774 | BLASTX | 598 | 7.00E−62 | 64 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 115 | LIB3602-012-Q6-K1-A6 | 548774 | BLASTX | 596 | 1.00E−61 | 60 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 116 | LIB3602-039-Q6-K1-G5 | 548774 | BLASTX | 592 | 3.00E−61 | 65 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 117 | LIB3602-107-Q1-K1-B7 | 548774 | BLASTX | 570 | 1.00E−58 | 59 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 118 | LIB3602-047-Q6-K1-C12 | 548774 | BLASTX | 568 | 2.00E−58 | 65 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 119 | LIB3602-094-Q6-K6-H9 | 548774 | BLASTX | 546 | 9.00E−56 | 56 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 120 | LIB3602-091-Q6-K6-H9 | 548774 | BLASTX | 539 | 6.00E−55 | 55 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 121 | LIB3602-108-Q1-K1-C12 | 548774 | BLASTX | 530 | 6.00E−54 | 58 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 122 | LIB3602-077-Q6-K6-H6 | 548774 | BLASTX | 526 | 2.00E−53 | 55 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 123 | LIB3602-116-Q1-K1-H9 | 548774 | BLASTX | 414 | 2.00E−53 | 62 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 124 | LIB3602-043-Q6-K1-G4 | 548774 | BLASTX | 524 | 3.00E−53 | 59 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 125 | LIB3602-052-Q6-K1-F5 | 548774 | BLASTX | 494 | 8.00E−50 | 64 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 126 | LIB3602-001-P1-K6-G6 | 548774 | BLASTX | 471 | 5.00E−47 | 59 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 127 | LIB3602-007-Q1-K1-C12 | 548774 | BLASTX | 459 | 2.00E−45 | 52 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 128 | LIB3602-006-Q1-K1-H5 | 548774 | BLASTX | 444 | 8.00E−44 | 52 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 129 | LIB3602-059-Q6-K1-F12 | 548774 | BLASTX | 427 | 7.00E−42 | 56 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 130 | LIB3602-103-Q1-K1-C12 | 548774 | BLASTX | 383 | 2.00E−41 | 59 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 131 | LIB3602-029-Q6-K1-F9 | 548774 | BLASTX | 414 | 2.00E−40 | 62 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 132 | LIB3602-019-Q6-K1-B5 | 548774 | BLASTX | 389 | 1.00E−37 | 72 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 133 | LIB3602-056-Q6-K1-B8 | 548774 | BLASTX | 386 | 3.00E−37 | 58 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 134 | LIB3602-080-Q6-K6-B9 | 548774 | BLASTX | 298 | 4.00E−36 | 60 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 135 | LIB3602-048-Q6-K1QA-B9 | 548774 | BLASTX | 374 | 8.00E−36 | 62 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 136 | LIB3602-053-Q6-K1-H1 | 548774 | BLASTX | 366 | 8.00E−35 | 63 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 137 | LIB3602-076-Q6-K6-H10 | 548774 | BLASTX | 357 | 1.00E−33 | 62 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 138 | LIB3602-063-Q1-K6-F1 | 548774 | BLASTX | 348 | 8.00E−33 | 54 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 139 | LIB3602-032-Q6-K1-E6 | 548774 | BLASTX | 315 | 6.00E−29 | 76 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 140 | LIB3602-077-Q6-K6-C12 | 548774 | BLASTX | 304 | 2.00E−27 | 62 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 141 | LIB3602-081-Q6-K6-E9 | 548774 | BLASTX | 166 | 2.00E−11 | 57 | 60S RIBOSOMAL PROTEIN L7A |
| | | | | | | | >gi\|542158\|pir\|\|S38360 ribosomal protein L7a, |
| | | | | | | | cytosolic - rice >gi\|303855\|dbj\|BAA02156.1\| |
| | | | | | | | (D12631) ribosomal protein L7A [Oryza sativa] |
| 142 | LIB3602-094-Q6-K6-B5 | 6440817 | BLASTX | 391 | 1.00E−37 | 62 | (AU066512) hypothetical protein [Chlamydomonas |
| | | | | | | | sp.] |
| 143 | LIB3602-054-Q6-K1-E10 | 6440817 | BLASTX | 383 | 8.00E−37 | 61 | (AU066512) hypothetical protein [Chlamydomonas |
| | | | | | | | sp.] |
| 144 | LIB3602-045-Q6-K1-E3 | 6440817 | BLASTX | 383 | 9.00E−37 | 61 | (AU066512) hypothetical protein [Chlamydomonas |
| | | | | | | | sp.] |
| 145 | LIB3602-103-Q1-K1-B9 | 6440817 | BLASTX | 383 | 9.00E−37 | 61 | (AU066512) hypothetical protein [Chlamydomonas |
| | | | | | | | sp.] |
| 146 | LIB3602-070-Q1-K1-D3 | 6440817 | BLASTX | 383 | 1.00E−36 | 61 | (AU066512) hypothetical protein [Chlamydomonas |
| | | | | | | | sp.] |
| 147 | LIB3602-094-Q6-K6-G5 | 6440817 | BLASTX | 383 | 1.00E−36 | 61 | (AU066512) hypothetical protein [Chlamydomonas |
| | | | | | | | sp.] |
| 148 | LIB3602-087-Q6-K1-G6 | 6440817 | BLASTX | 383 | 1.00E−36 | 61 | (AU066512) hypothetical protein [Chlamydomonas |
| | | | | | | | sp.] |
| 149 | LIB3602-104-Q1-K1-E12 | 6440817 | BLASTX | 383 | 1.00E−36 | 61 | (AU066512) hypothetical protein [Chlamydomonas |
| | | | | | | | sp.] |
| 150 | LIB3602-029-Q6-K1-F4 | 6440817 | BLASTX | 383 | 1.00E−36 | 61 | (AU066512) hypothetical protein [Chlamydomonas |
| | | | | | | | sp.] |
| 151 | LIB3602-065-Q1-K6-E9 | 6440817 | BLASTX | 383 | 1.00E−36 | 61 | (AU066512) hypothetical protein [Chlamydomonas |
| | | | | | | | sp.] |
| 152 | LIB3602-084-Q6-K1-E1 | 6440817 | BLASTX | 379 | 3.00E−36 | 60 | (AU066512) hypothetical protein [Chlamydomonas |
| | | | | | | | sp.] |
| 153 | LIB3602-066-Q1-K6-A4 | 6440817 | BLASTX | 379 | 4.00E−36 | 62 | (AU066512) hypothetical protein [Chlamydomonas |
| | | | | | | | sp.] |
| 154 | LIB3602-107-Q1-K1-C11 | 6440817 | BLASTX | 378 | 4.00E−36 | 64 | (AU066512) hypothetical protein [Chlamydomonas |
| | | | | | | | sp.] |
| 155 | LIB3602-060-Q6-K1-H8 | 6440817 | BLASTX | 377 | 5.00E−36 | 61 | (AU066512) hypothetical protein [Chlamydomonas |
| | | | | | | | sp.] |
| 156 | LIB3602-060-Q6-K1-F2 | 6440817 | BLASTX | 370 | 3.00E−35 | 60 | (AU066512) hypothetical protein [Chlamydomonas |
| | | | | | | | sp.] |
| 157 | LIB3602-058-Q6-K1-H10 | 6440817 | BLASTX | 365 | 1.00E−34 | 62 | (AU066512) hypothetical protein [Chlamydomonas |
| | | | | | | | sp.] |
| 158 | LIB3602-005-Q1-K1-E11 | 6440817 | BLASTX | 360 | 6.00E−34 | 63 | (AU066512) hypothetical protein [Chlamydomonas |
| | | | | | | | sp.] |
| 159 | LIB3602-048-Q6-K1QA-G5 | 6440817 | BLASTX | 354 | 2.00E−33 | 64 | (AU066512) hypothetical protein [Chlamydomonas |
| | | | | | | | sp.] |
| 160 | LIB3602-037-Q6-K1-H2 | 6440817 | BLASTX | 350 | 6.00E−33 | 73 | (AU066512) hypothetical protein [Chlamydomonas |
| | | | | | | | sp.] |
| 161 | LIB3602-111-Q1-K1-E3 | 6440817 | BLASTX | 339 | 6.00E−33 | 63 | (AU066512) hypothetical protein [Chlamydomonas |
| | | | | | | | sp.] |
| 162 | LIB3602-085-Q6-K1-E8 | 6440817 | BLASTX | 289 | 2.00E−29 | 66 | (AU066512) hypothetical protein [Chlamydomonas |
| | | | | | | | sp.] |
| 163 | LIB3602-079-Q6-K6-H11 | 6440817 | BLASTX | 290 | 8.00E−26 | 70 | (AU066512) hypothetical protein [Chlamydomonas |
| | | | | | | | sp.] |
| 164 | LIB3602-059-Q6-K1-B9 | 6440817 | BLASTX | 285 | 1.00E−25 | 72 | (AU066512) hypothetical protein [Chlamydomonas |
| | | | | | | | sp.] |
| 165 | LIB3602-071-Q1-K1-E7 | 6440817 | BLASTX | 277 | 2.00E−24 | 69 | (AU066512) hypothetical protein [Chlamydomonas |
| | | | | | | | sp.] |
| 166 | LIB3602-116-Q1-K1-B5 | 6440817 | BLASTX | 271 | 2.00E−23 | 78 | (AU066512) hypothetical protein [Chlamydomonas |
| | | | | | | | sp.] |
| 167 | LIB3602-012-Q6-K1-D10 | 6440817 | BLASTX | 263 | 1.00E−22 | 69 | (AU066512) hypothetical protein [Chlamydomonas |
| | | | | | | | sp.] |
| 168 | LIB3602-045-Q6-K1-E4 | 6440817 | BLASTX | 196 | 6.00E−15 | 44 | (AU066512) hypothetical protein [Chlamydomonas |
| | | | | | | | sp.] |
| 169 | LIB3602-049-Q6-K1-F3 | 6440817 | BLASTX | 184 | 2.00E−13 | 65 | (AU066512) hypothetical protein [Chlamydomonas |
| | | | | | | | sp.] |
| 170 | LIB3602-029-Q6-K1-F5 | 6440817 | BLASTX | 155 | 5.00E−10 | 75 | (AU066512) hypothetical protein [Chlamydomonas |
| | | | | | | | sp.] |
| 171 | LIB3602-022-Q6-K1-C12 | 485518 | BLASTX | 620 | 1.00E−64 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 172 | LIB3602-054-Q6-K1-C6 | 485518 | BLASTX | 620 | 1.00E−64 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 173 | LIB3602-079-Q6-K6-F2 | 485518 | BLASTX | 620 | 2.00E−64 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 174 | LIB3602-034-Q6-K1-F6 | 485518 | BLASTX | 620 | 2.00E−64 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 175 | LIB3602-038-Q6-K1-H6 | 485518 | BLASTX | 620 | 2.00E−64 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 176 | LIB3602-042-Q6-K1-D8 | 485518 | BLASTX | 620 | 2.00E−64 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 177 | LIB3602-077-Q6-K6-C3 | 485518 | BLASTX | 620 | 2.00E−64 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 178 | LIB3602-029-Q6-K1-G1 | 485518 | BLASTX | 620 | 2.00E−64 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 179 | LIB3602-111-Q1-K1-G1 | 485518 | BLASTX | 614 | 9.00E−64 | 95 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 180 | LIB3602-112-Q1-K1-E1 | 485518 | BLASTX | 611 | 2.00E−63 | 95 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 181 | LIB3602-056-Q6-K1-G9 | 485518 | BLASTX | 594 | 1.00E−61 | 90 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 182 | LIB3602-035-Q1-K1-F6 | 485518 | BLASTX | 588 | 1.00E−60 | 94 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 183 | LIB3602-023-Q6-K1-B9 | 485518 | BLASTX | 582 | 5.00E−60 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 184 | LIB3602-091-Q6-K6-B8 | 485518 | BLASTX | 577 | 2.00E−59 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 185 | LIB3602-041-Q6-K1-D4 | 485518 | BLASTX | 577 | 2.00E−59 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 186 | LIB3602-085-Q6-K1-F1 | 485518 | BLASTX | 572 | 8.00E−59 | 95 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 187 | LIB3602-005-Q1-K1-C4 | 485518 | BLASTX | 560 | 2.00E−57 | 93 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 188 | LIB3602-108-Q1-K1-B6 | 485518 | BLASTX | 411 | 2.00E−57 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 189 | LIB3602-047-Q6-K1-D10 | 485518 | BLASTX | 553 | 1.00E−56 | 95 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 190 | LIB3602-101-Q1-K1-D1 | 485518 | BLASTX | 420 | 2.00E−56 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 191 | LIB3602-009-Q6-K1-B12 | 485518 | BLASTX | 511 | 9.00E−52 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 192 | LIB3602-094-Q6-K6-D11 | 485518 | BLASTX | 504 | 7.00E−51 | 95 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 193 | LIB3602-113-Q1-K1-A5 | 485518 | BLASTX | 482 | 2.00E−48 | 94 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 194 | LIB3602-112-Q1-K1-A5 | 485518 | BLASTX | 482 | 3.00E−48 | 94 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 195 | LIB3602-108-Q1-K1-D2 | 485518 | BLASTX | 298 | 3.00E−47 | 93 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 196 | LIB3602-032-Q6-K1-F10 | 485518 | BLASTX | 470 | 5.00E−47 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 197 | LIB3602-025-Q6-K1-G12 | 485518 | BLASTX | 456 | 2.00E−45 | 89 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 198 | LIB3602-038-Q6-K1-F12 | 485518 | BLASTX | 423 | 8.00E−42 | 92 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 199 | LIB3602-014-Q6-K1-G3 | 485518 | BLASTX | 386 | 3.00E−37 | 96 | ubiquitin/ribosomal protein CEP52 - rice |
| | | | | | | | >gi\|303857\|dbj\|BAA02154.1\| (D12629) |
| | | | | | | | ubiquitin/ribosomal polyprotein [Oryza sativa] |
| 200 | LIB3602-079-Q6-K6-B12 | 7440926 | BLASTX | 715 | 1.00E−75 | 73 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 201 | LIB3602-065-Q1-K6-E3 | 7440926 | BLASTX | 712 | 3.00E−75 | 72 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 202 | LIB3602-002-P1-K6-B1 | 7440926 | BLASTX | 712 | 3.00E−75 | 72 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 203 | LIB3602-023-Q6-K1-F5 | 7440926 | BLASTX | 690 | 1.00E−72 | 74 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 204 | LIB3602-105-Q1-K1-B12 | 7440926 | BLASTX | 669 | 3.00E−70 | 68 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 205 | LIB3602-028-Q6-K1-B11 | 7440926 | BLASTX | 646 | 1.00E−67 | 74 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 206 | LIB3602-045-Q6-K1-A3 | 7440926 | BLASTX | 625 | 4.00E−65 | 76 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 207 | LIB3602-045-Q6-K1-B11 | 7440926 | BLASTX | 611 | 2.00E−63 | 77 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 208 | LIB3602-056-Q6-K1-D10 | 7440926 | BLASTX | 603 | 1.00E−62 | 75 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 209 | LIB3602-091-Q6-K6-H8 | 7440926 | BLASTX | 410 | 2.00E−61 | 69 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 210 | LIB3602-094-Q6-K6-H8 | 7440926 | BLASTX | 420 | 5.00E−61 | 71 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 211 | LIB3602-032-Q6-K1-A3 | 7440926 | BLASTX | 588 | 7.00E−61 | 79 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 212 | LIB3602-115-Q1-K1-H9 | 7440926 | BLASTX | 420 | 1.00E−60 | 71 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 213 | LIB3602-019-Q6-K1-B10 | 7440926 | BLASTX | 566 | 3.00E−58 | 74 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 214 | LIB3602-101-Q1-K1-B10 | 7440926 | BLASTX | 547 | 4.00E−56 | 74 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 215 | LIB3602-072-Q1-K1-D6 | 7440926 | BLASTX | 420 | 5.00E−55 | 71 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 216 | LIB3602-037-Q6-K1-G3 | 7440926 | BLASTX | 503 | 1.00E−54 | 79 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 217 | LIB3602-068-Q1-K1-C4 | 7440926 | BLASTX | 364 | 6.00E−52 | 65 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 218 | LIB3602-028-Q6-K1-A7 | 7440926 | BLASTX | 492 | 1.00E−49 | 72 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 219 | LIB3602-046-Q6-K1-G4 | 7440926 | BLASTX | 489 | 3.00E−49 | 71 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 220 | LIB3602-023-Q6-K1-F3 | 7440926 | BLASTX | 395 | 4.00E−38 | 71 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 221 | LIB3602-015-Q6-K1-D2 | 7440926 | BLASTX | 333 | 7.00E−31 | 69 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 222 | LIB3602-028-Q6-K1-H3 | 7440926 | BLASTX | 333 | 7.00E−31 | 69 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 223 | LIB3602-021-Q6-K1-A12 | 7440926 | BLASTX | 320 | 8.00E−30 | 73 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 224 | LIB3602-028-Q6-K1-B5 | 7440926 | BLASTX | 253 | 3.00E−22 | 83 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 225 | LIB3602-003-Q1-K1-H9 | 7440926 | BLASTX | 254 | 9.00E−22 | 64 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 226 | LIB3602-089-Q6-K6-A11 | 7440926 | BLASTX | 171 | 5.00E−12 | 56 | ribosomal protein L18a, cytosolic - Arabidopsis |
| | | | | | | | thaliana >gi\|3128228\|gb\|AAC26708.1\| (AC004077) |
| | | | | | | | 60S ribosomal protein L18A [Arabidopsis thaliana] |
| 227 | LIB3602-094-Q6-K6-B6 | 266945 | BLASTX | 606 | 8.00E−63 | 64 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 228 | LIB3602-095-Q6-K6-B6 | 266945 | BLASTX | 601 | 3.00E−62 | 64 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 229 | LIB3602-089-Q6-K6-B6 | 266945 | BLASTX | 595 | 2.00E−61 | 64 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 230 | LIB3602-018-Q6-K1-C6 | 266945 | BLASTX | 587 | 1.00E−60 | 63 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 231 | LIB3602-015-Q6-K1-D1 | 266945 | BLASTX | 586 | 2.00E−60 | 65 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 232 | LIB3602-073-Q1-K1-F6 | 266945 | BLASTX | 584 | 3.00E−60 | 64 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 233 | LIB3602-113-Q1-K1-F7 | 266945 | BLASTX | 583 | 4.00E−60 | 64 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 234 | LIB3602-018-Q6-K1-C5 | 266945 | BLASTX | 581 | 6.00E−60 | 63 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 235 | LIB3602-073-Q1-K1-C6 | 266945 | BLASTX | 579 | 1.00E−59 | 64 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 236 | LIB3602-091-Q6-K6-G2 | 266945 | BLASTX | 567 | 3.00E−58 | 64 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 237 | LIB3602-004-Q1-K1-B9 | 266945 | BLASTX | 566 | 5.00E−58 | 65 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 238 | LIB3602-069-Q1-K1-B6 | 266945 | BLASTX | 533 | 3.00E−54 | 66 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 239 | LIB602-015-Q6-K1-D8 | 266945 | BLASTX | 528 | 1.00E−53 | 66 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 240 | LIB3602-076-Q6-K6-E6 | 266945 | BLASTX | 521 | 8.00E−53 | 66 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 241 | LIB3602-011-Q6-K1-B8 | 266945 | BLASTX | 518 | 2.00E−52 | 62 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 242 | LIB3602-102-Q1-K1-A8 | 266945 | BLASTX | 515 | 3.00E−52 | 66 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 243 | LIB3602-070-Q1-K1-B6 | 266945 | BLASTX | 513 | 7.00E−52 | 66 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 244 | LIB3602-045-Q6-K1-A6 | 266945 | BLASTX | 505 | 4.00E−51 | 65 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 245 | LIB3602-041-Q6-K1-H1 | 266945 | BLASTX | 494 | 9.00E−50 | 67 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 246 | LIB3602-045-Q6-K1-G6 | 266945 | BLASTX | 487 | 6.00E−49 | 63 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 247 | LIB3602-013-Q6-K1-F2 | 266945 | BLASTX | 486 | 7.00E−49 | 63 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 248 | LIB3602-012-Q6-K1-C2 | 266945 | BLASTX | 463 | 5.00E−46 | 62 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 249 | LIB3602-092-Q6-K6-D9 | 266945 | BLASTX | 203 | 4.00E−29 | 57 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 250 | LIB3602-015-Q6-K1-D9 | 266945 | BLASTX | 214 | 2.00E−21 | 42 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 251 | LIB3602-115-Q1-K1-E1 | 266945 | BLASTX | 192 | 9.00E−15 | 74 | 60S RIBOSOMAL PROTEIN L9 (GIBBERELLIN- |
| | | | | | | | REGULATED PROTEIN GA) |
| | | | | | | | >gi\|100065\|pir\|\|S19978 ribosomal protein L9, |
| | | | | | | | cytosolic - garden pea >gi\|20727\|emb\|CAA46273.1\| |
| | | | | | | | (X65155) GA [Pisum sativum] |
| 252 | LIB3602-100-Q1-K1-B9 | 115826 | BLASTX | 489 | 4.00E−49 | 77 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 253 | LIB3602-107-Q1-K1-D5 | 115826 | BLASTX | 464 | 4.00E−46 | 77 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 254 | LIB3602-044-Q6-K1-B10 | 115826 | BLASTX | 463 | 4.00E−46 | 77 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 255 | LIB3602-115-Q1-K1-D6 | 115826 | BLASTX | 414 | 4.00E−41 | 80 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 256 | LIB3602-070-Q1-K1-G8 | 115826 | BLASTX | 414 | 1.00E−40 | 80 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 257 | LIB3602-102-Q1-K1-A2 | 115826 | BLASTX | 302 | 2.00E−40 | 82 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 258 | LIB3602-118-Q1-K1-D5 | 115826 | BLASTX | 211 | 3.00E−34 | 86 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 259 | LIB3602-044-Q6-K1-F3 | 115826 | BLASTX | 344 | 3.00E−32 | 76 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 260 | LIB3602-052-Q6-K1-D11 | 115826 | BLASTX | 331 | 9.00E−31 | 77 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 261 | LIB3602-083-Q6-K6-E3 | 115826 | BLASTX | 330 | 1.00E−30 | 61 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 262 | LIB3602-118-Q1-K1-F7 | 115826 | BLASTX | 329 | 2.00E−30 | 79 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 263 | LIB3602-037-Q6-K1-C3 | 115826 | BLASTX | 329 | 2.00E−30 | 79 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 264 | LIB3602-022-Q6-K1-H7 | 115826 | BLASTX | 319 | 2.00E−29 | 76 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 265 | LIB3602-112-Q1-K1-A10 | 115826 | BLASTX | 319 | 2.00E−29 | 76 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 266 | LIB3602-089-Q6-K6-A10 | 115826 | BLASTX | 315 | 6.00E−29 | 75 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 267 | LIB3602-017-Q6-K1-A10 | 115826 | BLASTX | 293 | 2.00E−26 | 75 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 268 | LIB3602-004-Q1-K1-B10 | 115826 | BLASTX | 278 | 1.00E−24 | 57 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 269 | LIB3602-027-Q6-K1-G9 | 115826 | BLASTX | 266 | 3.00E−23 | 78 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 270 | LIB3602-106-Q1-K1-B9 | 115826 | BLASTX | 225 | 2.00E−20 | 49 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 271 | LIB3602-044-Q6-K1-G12 | 115826 | BLASTX | 224 | 2.00E−18 | 76 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 272 | LIB3602-063-Q1-K6-E3 | 115826 | BLASTX | 223 | 3.00E−18 | 74 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 273 | LIB3602-067-Q1-K1-A10 | 115826 | BLASTX | 193 | 9.00E−16 | 70 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 274 | LIB3602-058-Q6-K1-C11 | 115826 | BLASTX | 201 | 1.00E−15 | 74 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 275 | LIB3602-029-Q6-K1-B10 | 115826 | BLASTX | 201 | 1.00E−15 | 74 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 276 | LIB3602-054-Q6-K1-E4 | 115826 | BLASTX | 162 | 5.00E−11 | 70 | CHLOROPHYLL A-B BINDING PROTEIN OF |
| | | | | | | | LHCII TYPE I PRECURSOR (CAB) (LHCP) |
| | | | | | | | >gi\|99371\|pir\|\|S14518 chlorophyll a/b-binding |
| | | | | | | | protein - Chlamydomonas moewusii |
| | | | | | | | >gi\|18063\|emb\|CAA38635.1\| (X54856) chlorophyll |
| | | | | | | | a/b-binding protein [Chlamydomonas moewusii] |
| 277 | LIB3602-015-Q6-K1-G2 | 7440560 | BLASTX | 296 | 8.00E−27 | 73 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 278 | LIB3602-017-Q6-K1-G10 | 7440560 | BLASTX | 292 | 2.00E−26 | 72 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 279 | LIB3602-107-Q1-K1-C9 | 7440560 | BLASTX | 272 | 6.00E−24 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 280 | LIB3602-039-Q6-K1-H8 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 281 | LIB3602-049-Q6-K1-D5 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 282 | LIB3602-042-Q6-K1-B2 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 283 | LIB3602-061-Q6-K1-H5 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 284 | LIB3602-077-Q6-K6-F9 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 285 | LIB3602-079-Q6-K6-A3 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 286 | LIB3602-047-Q6-K1-A2 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 287 | LIB3602-017-Q6-K1-F8 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 288 | LIB3602-017-Q6-K1-C12 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 289 | LIB3602-018-Q6-K1-E12 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 290 | LIB3602-012-Q6-K1-E12 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 291 | LIB3602-026-Q6-K1-A12 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 292 | LIB3602-026-Q6-K1-A5 | 7440560 | BLASTX | 247 | 6.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 293 | LIB3602-015-Q6-K1-C12 | 7440560 | BLASTX | 247 | 7.00E−21 | 71 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 294 | LIB3602-109-Q1-K1-H2 | 7440560 | BLASTX | 239 | 5.00E−20 | 69 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 295 | LIB3602-109-Q1-K1-C2 | 7440560 | BLASTX | 238 | 6.00E−20 | 69 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 296 | LIB3602-101-Q1-K1-A1 | 7440560 | BLASTX | 237 | 9.00E−20 | 68 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 297 | LIB3602-087-Q6-K1-H9 | 7440560 | BLASTX | 229 | 1.00E−18 | 70 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 298 | LIB3602-021-Q6-K1-D5 | 7440560 | BLASTX | 215 | 2.00E−17 | 76 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 299 | LIB3602-106-Q1-K1-D12 | 7440560 | BLASTX | 212 | 1.00E−16 | 74 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 300 | LIB3602-058-Q6-K1-C7 | 7440560 | BLASTX | 160 | 3.00E−11 | 67 | ribosomal protein S25, cytosolic - Arabidopsis |
| | | | | | | | thaliana >gi\|4914432\|emb\|CAB43635.1\| |
| | | | | | | | (AL050351) ribosomal protein S25 [Arabidopsis |
| | | | | | | | thaliana] >gi\|7270903\|emb\|CAB80583.1\| |
| | | | | | | | (AL161594) ribosomal protein S25 [Arabidopsis |
| | | | | | | | thaliana] |
| 301 | LIB3602-017-Q6-K1-E11 | 6094037 | BLASTX | 595 | 1.00E−61 | 81 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 302 | LIB3602-067-Q1-K1-H1 | 6094037 | BLASTX | 595 | 2.00E−61 | 81 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 303 | LIB3602-074-Q1-K1-H1 | 6094037 | BLASTX | 595 | 2.00E−61 | 81 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 304 | LIB3602-041-Q6-K1-G12 | 6094037 | BLASTX | 591 | 5.00E−61 | 81 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 305 | LIB3602-007-Q1-K1-B11 | 6094037 | BLASTX | 573 | 5.00E−59 | 84 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 306 | LIB3602-069-Q1-K1-H8 | 6094037 | BLASTX | 564 | 7.00E−58 | 85 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 307 | LIB3602-050-Q6-K1-A1 | 6094037 | BLASTX | 560 | 1.00E−57 | 84 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 308 | LIB3602-009-Q6-K1-H7 | 6094037 | BLASTX | 536 | 8.00E−55 | 82 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 309 | LIB3602-053-Q6-K1-G11 | 6094037 | BLASTX | 526 | 1.00E−53 | 73 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 310 | LIB3602-014-Q6-K1-F6 | 6094037 | BLASTX | 513 | 4.00E−52 | 80 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 311 | LIB3602-009-Q6-K1-H8 | 6094037 | BLASTX | 487 | 5.00E−49 | 85 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 312 | LIB3602-023-Q6-K1-H4 | 6094037 | BLASTX | 487 | 6.00E−49 | 85 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 313 | LIB3602-042-Q6-K1-D1 | 6094037 | BLASTX | 473 | 2.00E−47 | 84 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 314 | LIB3602-101-Q1-K1-A6 | 6094037 | BLASTX | 391 | 1.00E−46 | 87 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 315 | LIB3602-002-P1-K6-B12 | 6094037 | BLASTX | 450 | 1.00E−44 | 73 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 316 | LIB3602-039-Q6-K1-C12 | 6094037 | BLASTX | 437 | 3.00E−43 | 85 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 317 | LIB3602-028-Q6-K1-D5 | 6094037 | BLASTX | 437 | 3.00E−43 | 85 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 318 | LIB3602-018-Q6-K1-B9 | 6094037 | BLASTX | 386 | 2.00E−37 | 86 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 319 | LIB3602-060-Q6-K1-C8 | 6094037 | BLASTX | 369 | 3.00E−35 | 79 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 320 | LIB3602-004-Q1-K1-D2 | 6094037 | BLASTX | 345 | 2.00E−32 | 80 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 321 | LIB3602-071-Q1-K1-A3 | 6094037 | BLASTX | 342 | 7.00E−32 | 84 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 322 | LIB3602-106-Q1-K1-G9 | 6094037 | BLASTX | 291 | 4.00E−26 | 72 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 323 | LIB3602-001-P1-K6-H2 | 6094037 | BLASTX | 261 | 7.00E−23 | 84 | 60S RIBOSOMAL PROTEIN L23 |
| | | | | | | | >gi\|7440854\|pir\|\|T11639 ribosomal protein L17 - |
| | | | | | | | fission yeast (Schizosaccharomyces pombe) |
| | | | | | | | >gi\|7490001\|pir\|\|T40942 60s ribosomal protein - |
| | | | | | | | fission yeast (Schizosaccharomyces pombe) |
| | | | | | | | >gi\|2706454\|emb\|CAA15912.1\| (AL021046) 60s |
| | | | | | | | riboso |
| 324 | LIB3602-081-Q6-K6-E6 | 730557 | BLASTX | 388 | 2.00E−37 | 62 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 325 | LIB3602-019-Q6-K1-B8 | 730557 | BLASTX | 348 | 1.00E−32 | 64 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 326 | LIB3602-043-Q6-K1-D3 | 730557 | BLASTX | 316 | 6.00E−29 | 61 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 327 | LIB3602-046-Q6-K1-A4 | 730557 | BLASTX | 316 | 6.00E−29 | 61 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 328 | LIB3602-061-Q6-K1-G10 | 730557 | BLASTX | 316 | 6.00E−29 | 61 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 329 | LIB3602-074-Q1-K1-G5 | 730557 | BLASTX | 316 | 6.00E−29 | 61 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 330 | LIB3602-062-Q6-K1-F4 | 730557 | BLASTX | 316 | 6.00E−29 | 61 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 331 | LIB3602-008-Q6-K1-C9 | 730557 | BLASTX | 316 | 6.00E−29 | 61 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 332 | LIB3602-022-Q6-K1-D8 | 730557 | BLASTX | 316 | 6.00E−29 | 61 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 333 | LIB3602-076-Q6-K6-D11 | 730557 | BLASTX | 316 | 7.00E−29 | 61 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 334 | LIB3602-011-Q6-K1-H4 | 730557 | BLASTX | 316 | 7.00E−29 | 61 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 335 | LIB3602-041-Q6-K1-F12 | 730557 | BLASTX | 316 | 7.00E−29 | 61 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 336 | LIB3602-028-Q6-K1-H4 | 730557 | BLASTX | 316 | 7.00E−29 | 61 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 337 | LIB3602-063-Q1-K6-H3 | 730557 | BLASTX | 314 | 1.00E−28 | 57 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 338 | LIB3602-074-Q1-K1-B3 | 730557 | BLASTX | 311 | 2.00E−28 | 60 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 339 | LIB3602-087-Q6-K1-D4 | 730557 | BLASTX | 247 | 2.00E−22 | 55 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 340 | LIB3602-119-Q1-K1-G1 | 730557 | BLASTX | 248 | 2.00E−21 | 54 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 341 | LIB3602-041-Q6-K1-C10 | 730557 | BLASTX | 229 | 8.00E−19 | 52 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 342 | LIB3602-069-Q1-K1-B3 | 730557 | BLASTX | 226 | 2.00E−18 | 57 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 343 | LIB3602-085-Q6-K1-D11 | 730557 | BLASTX | 189 | 4.00E−14 | 51 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 344 | LIB3602-102-Q1-K1-E11 | 730557 | BLASTX | 150 | 6.00E−10 | 78 | 60S RIBOSOMAL PROTEIN L34 |
| | | | | | | | >gi\|2119150\|pir\|\|S60476 ribosomal protein L34, |
| | | | | | | | cytosolic - garden pea >gi\|498908\|gb\|AAA86953.1\| |
| | | | | | | | (U10047) ribosomal protein L34 homolog [Pisum |
| | | | | | | | sativum] |
| 345 | LIB3602-067-Q1-K1-E6 | 2894306 | BLASTX | 338 | 2.00E−31 | 46 | (AJ223329) ubiquitin extension protein [Nicotiana |
| | | | | | | | tabacum] |
| 346 | LIB3602-023-Q6-K1-C11 | 2894306 | BLASTX | 337 | 2.00E−31 | 47 | (AJ223329) ubiquitin extension protein [Nicotiana |
| | | | | | | | tabacum] |
| 347 | LIB3602-007-Q1-K1-C11 | 2894306 | BLASTX | 338 | 3.00E−31 | 46 | (AJ223329) ubiquitin extension protein [Nicotiana |
| | | | | | | | tabacum] |
| 348 | LIB3602-094-Q6-K6-E10 | 2894306 | BLASTX | 337 | 3.00E−31 | 47 | (AJ223329) ubiquitin extension protein [Nicotiana |
| | | | | | | | tabacum] |
| 349 | LIB3602-041-Q6-K1-B5 | 2894306 | BLASTX | 337 | 3.00E−31 | 47 | (AJ223329) ubiquitin extension protein [Nicotiana |
| | | | | | | | tabacum] |
| 350 | LIB3602-062-Q6-K1-G10 | 2894306 | BLASTX | 317 | 6.00E−29 | 45 | (AJ223329) ubiquitin extension protein [Nicotiana |
| | | | | | | | tabacum] |
| 351 | LIB3602-113-Q1-K1-F8 | 2894306 | BLASTX | 271 | 1.00E−23 | 50 | (AJ223329) ubiquitin extension protein [Nicotiana |
| | | | | | | | tabacum] |
| 352 | LIB3602-020-Q6-K1-E6 | 2894306 | BLASTX | 253 | 2.00E−21 | 50 | (AJ223329) ubiquitin extension protein [Nicotiana |
| | | | | | | | tabacum] |
| 353 | LIB3602-046-Q6-K1-F2 | 2894306 | BLASTX | 249 | 4.00E−21 | 50 | (AJ223329) ubiquitin extension protein [Nicotiana |
| | | | | | | | tabacum] |
| 354 | LIB3602-101-Q1-K1-H7 | 2894306 | BLASTX | 213 | 1.00E−19 | 47 | (AJ223329) ubiquitin extension protein [Nicotiana |
| | | | | | | | tabacum] |
| 355 | LIB3602-039-Q6-K1-H3 | 2894306 | BLASTX | 218 | 2.00E−17 | 48 | (AJ223329) ubiquitin extension protein [Nicotiana |
| | | | | | | | tabacum] |
| 356 | LIB3602-115-Q1-K1-H6 | 2894306 | BLASTX | 217 | 3.00E−17 | 47 | (AJ223329) ubiquitin extension protein [Nicotiana |
| | | | | | | | tabacum] |
| 357 | LIB3602-002-P1-K6-C12 | 2894306 | BLASTX | 215 | 4.00E−17 | 49 | (AJ223329) ubiquitin extension protein [Nicotiana |
| | | | | | | | tabacum] |
| 358 | LIB3602-045-Q6-K1-A12 | 2894306 | BLASTX | 215 | 4.00E−17 | 49 | (AJ223329) ubiquitin extension protein [Nicotiana |
| | | | | | | | tabacum] |
| 359 | LIB3602-018-Q6-K1-G11 | 2894306 | BLASTX | 210 | 2.00E−16 | 50 | (AJ223329) ubiquitin extension protein [Nicotiana |
| | | | | | | | tabacum] |
| 360 | LIB3602-063-Q1-K6-D10 | 2894306 | BLASTX | 208 | 3.00E−16 | 50 | (AJ223329) ubiquitin extension protein [Nicotiana |
| | | | | | | | tabacum] |
| 361 | LIB3602-090-Q6-K6-D11 | 2894306 | BLASTX | 208 | 3.00E−16 | 50 | (AJ223329) ubiquitin extension protein [Nicotiana |
| | | | | | | | tabacum] |
| 362 | LIB3602-094-Q6-K6-G4 | 2894306 | BLASTX | 208 | 3.00E−16 | 50 | (AJ223329) ubiquitin extension protein [Nicotiana |
| | | | | | | | tabacum] |
| 363 | LIB3602-066-Q1-K6-C10 | 2894306 | BLASTX | 208 | 3.00E−16 | 50 | (AJ223329) ubiquitin extension protein [Nicotiana |
| | | | | | | | tabacum] |
| 364 | LIB3602-100-Q1-K1-F12 | 2894306 | BLASTX | 203 | 8.00E−16 | 79 | (AJ223329) ubiquitin extension protein [Nicotiana |
| | | | | | | | tabacum] |
| 365 | LIB3602-060-Q6-K1-F12 | 4204192 | BLASTX | 461 | 6.00E−46 | 82 | (AB008095) ribosomal protein L41 [Candida |
| | | | | | | | maltosa] |
| 366 | LIB3602-032-Q6-K1-D4 | 4204192 | BLASTX | 433 | 9.00E−43 | 82 | (AB008095) ribosomal protein L41 [Candida |
| | | | | | | | maltosa] |
| 367 | LIB3602-055-Q6-K1-B3 | 4204192 | BLASTX | 433 | 1.00E−42 | 82 | (AB008095) ribosomal protein L41 [Candida |
| | | | | | | | maltosa] |
| 368 | LIB3602-101-Q1-K1-F11 | 4204192 | BLASTX | 433 | 1.00E−42 | 82 | (AB008095) ribosomal protein L41 [Candida |
| | | | | | | | maltosa] |
| 369 | LIB3602-061-Q6-K1-F1 | 4204192 | BLASTX | 433 | 1.00E−42 | 82 | (AB008095) ribosomal protein L41 [Candida |
| | | | | | | | maltosa] |
| 370 | LIB3602-041-Q6-K1-C7 | 4204192 | BLASTX | 433 | 1.00E−42 | 82 | (AB008095) ribosomal protein L41 [Candida |
| | | | | | | | maltosa] |
| 371 | LIB3602-004-Q1-K1-G6 | 4204192 | BLASTX | 433 | 1.00E−42 | 82 | (AB008095) ribosomal protein L41 [Candida |
| | | | | | | | maltosa] |
| 372 | LIB3602-009-Q6-K1-H11 | 4204192 | BLASTX | 433 | 1.00E−42 | 82 | (AB008095) ribosomal protein L41 [Candida |
| | | | | | | | maltosa] |
| 373 | LIB3602-012-Q6-K1-A5 | 4204192 | BLASTX | 433 | 1.00E−42 | 82 | (AB008095) ribosomal protein L41 [Candida |
| | | | | | | | maltosa] |
| 374 | LIB3602-013-Q6-K1-F7 | 4204192 | BLASTX | 433 | 1.00E−42 | 82 | (AB008095) ribosomal protein L41 [Candida |
| | | | | | | | maltosa] |
| 375 | LIB3602-023-Q6-K1-H12 | 4204192 | BLASTX | 433 | 1.00E−42 | 82 | (AB008095) ribosomal protein L41 [Candida |
| | | | | | | | maltosa] |
| 376 | LIB3602-025-Q6-K1-C11 | 4204192 | BLASTX | 428 | 5.00E−42 | 82 | (AB008095) ribosomal protein L41 [Candida |
| | | | | | | | maltosa] |
| 377 | LIB3602-102-Q1-K1-E1 | 4204192 | BLASTX | 424 | 1.00E−41 | 82 | (AB008095) ribosomal protein L41 [Candida |
| | | | | | | | maltosa] |
| 378 | LIB3602-041-Q6-K1-E6 | 4204192 | BLASTX | 424 | 1.00E−41 | 80 | (AB008095) ribosomal protein L41 [Candida |
| | | | | | | | maltosa] |
| 379 | LIB3602-038-Q6-K1-F4 | 4204192 | BLASTX | 424 | 2.00E−41 | 82 | (AB008095) ribosomal protein L41 [Candida |
| | | | | | | | maltosa] |
| 380 | LIB3602-065-Q1-K6-A7 | 4204192 | BLASTX | 418 | 1.00E−40 | 80 | (AB008095) ribosomal protein L41 [Candida |
| | | | | | | | maltosa] |
| 381 | LIB3602-051-Q6-K1-H9 | 4204192 | BLASTX | 408 | 1.00E−39 | 79 | (AB008095) ribosomal protein L41 [Candida |
| | | | | | | | maltosa] |
| 382 | LIB3602-085-Q6-K1-H4 | 4204192 | BLASTX | 397 | 2.00E−38 | 79 | (AB008095) ribosomal protein L41 [Candida |
| | | | | | | | maltosa] |
| 383 | LIB3602-067-Q1-K1-E8 | 4204192 | BLASTX | 397 | 2.00E−38 | 79 | (AB008095) ribosomal protein L41 [Candida |
| | | | | | | | maltosa] |
| 384 | LIB3602-108-Q1-K1-E12 | 4204192 | BLASTX | 205 | 2.00E−27 | 75 | (AB008095) ribosomal protein L41 [Candida |
| | | | | | | | maltosa] |
| 385 | LIB3602-008-Q6-K1-F7 | 464707 | BLASTX | 582 | 4.00E−60 | 80 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 386 | LIB3602-039-Q6-K1-C9 | 464707 | BLASTX | 582 | 5.00E−60 | 80 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 387 | LIB3602-070-Q1-K1-C1 | 464707 | BLASTX | 582 | 5.00E−60 | 80 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 388 | LIB3602-038-Q6-K1-D2 | 464707 | BLASTX | 582 | 5.00E−60 | 80 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 389 | LIB3602-105-Q1-K1-D4 | 464707 | BLASTX | 582 | 5.00E−60 | 80 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 390 | LIB3602-076-Q6-K6-C7 | 464707 | BLASTX | 582 | 6.00E−60 | 80 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 391 | LIB3602-112-Q1-K1-G5 | 464707 | BLASTX | 582 | 6.00E−60 | 80 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 392 | LIB3602-093-Q6-K6-H10 | 464707 | BLASTX | 565 | 6.00E−58 | 80 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 393 | LIB3602-057-Q6-K1-G6 | 464707 | BLASTX | 457 | 2.00E−54 | 80 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 394 | LIB3602-107-Q1-K1-F3 | 464707 | BLASTX | 481 | 7.00E−48 | 80 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 395 | LIB3602-038-Q6-K1-D3 | 464707 | BLASTX | 470 | 7.00E−47 | 75 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 396 | LIB3602-109-Q1-K1-G3 | 464707 | BLASTX | 460 | 9.00E−46 | 80 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 397 | LIB3602-071-Q1-K1-C10 | 464707 | BLASTX | 421 | 4.00E−41 | 80 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 398 | LIB3602-060-Q6-K1-C12 | 464707 | BLASTX | 414 | 1.00E−40 | 82 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 399 | LIB3602-087-Q6-K1-B11 | 464707 | BLASTX | 367 | 8.00E−35 | 75 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 400 | LIB3602-104-Q1-K1-H10 | 464707 | BLASTX | 342 | 7.00E−32 | 55 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 401 | LIB3602-014-Q6-K1-C1 | 464707 | BLASTX | 340 | 9.00E−32 | 83 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 402 | LIB3602-037-Q6-K1-D10 | 464707 | BLASTX | 310 | 3.00E−28 | 87 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 403 | LIB3602-054-Q6-K1-B12 | 464707 | BLASTX | 217 | 2.00E−17 | 88 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 404 | LIB3602-108-Q1-K1-E5 | 464707 | BLASTX | 150 | 9.00E−10 | 93 | 40S RIBOSOMAL PROTEIN S18 |
| | | | | | | | >gi\|480908\|pir\|\|S37496 ribosomal protein S18.A, |
| | | | | | | | cytosolic - Arabidopsis thaliana |
| | | | | | | | >gi\|405613\|emb\|CAA80684.1\| (Z23165) ribosomal |
| | | | | | | | protein S18A [Arabidopsis thaliana] |
| | | | | | | | >gi\|434343\|emb\|CAA82273.1\| (Z28701) S18 |
| | | | | | | | ribosomal protein [Arabido |
| 405 | LIB3602-094-Q6-K6-G9 | 7413650 | BLASTX | 309 | 4.00E−28 | 55 | (AL162971) ribosomal protein L35-like |
| | | | | | | | [Arabidopsis thaliana] |
| 406 | LIB3602-091-Q6-K6-G9 | 7413650 | BLASTX | 309 | 4.00E−28 | 55 | (AL162971) ribosomal protein L35-like |
| | | | | | | | [Arabidopsis thaliana] |
| 407 | LIB3602-055-Q6-K1-F5 | 7413650 | BLASTX | 309 | 4.00E−28 | 55 | (AL162971) ribosomal protein L35-like |
| | | | | | | | [Arabidopsis thaliana] |
| 408 | LIB3602-079-Q6-K6-G1 | 7413650 | BLASTX | 309 | 4.00E−28 | 55 | (AL162971) ribosomal protein L35-like |
| | | | | | | | [Arabidopsis thaliana] |
| 409 | LIB3602-012-Q6-K1-C9 | 7413650 | BLASTX | 309 | 4.00E−28 | 55 | (AL162971) ribosomal protein L35-like |
| | | | | | | | [Arabidopsis thaliana] |
| 410 | LIB3602-032-Q6-K1-C9 | 7413650 | BLASTX | 298 | 8.00E−27 | 55 | (AL162971) ribosomal protein L35-like |
| | | | | | | | [Arabidopsis thaliana] |
| 411 | LIB3602-104-Q1-K1-E5 | 7413650 | BLASTX | 290 | 6.00E−26 | 54 | (AL162971) ribosomal protein L35-like |
| | | | | | | | [Arabidopsis thaliana] |
| 412 | LIB3602-014-Q6-K1-E12 | 7413650 | BLASTX | 288 | 1.00E−25 | 66 | (AL162971) ribosomal protein L35-like |
| | | | | | | | [Arabidopsis thaliana] |
| 413 | LIB3602-090-Q6-K6-G2 | 7413650 | BLASTX | 286 | 2.00E−25 | 53 | (AL162971) ribosomal protein L35-like |
| | | | | | | | [Arabidopsis thaliana] |
| 414 | LIB3602-058-Q6-K1-H4 | 7413650 | BLASTX | 282 | 4.00E−25 | 65 | (AL162971) ribosomal protein L35-like |
| | | | | | | | [Arabidopsis thaliana] |
| 415 | LIB3602-094-Q6-K6-G12 | 7413650 | BLASTX | 271 | 1.00E−23 | 51 | (AL162971) ribosomal protein L35-like |
| | | | | | | | [Arabidopsis thaliana] |
| 416 | LIB3602-085-Q6-K1-G11 | 7413650 | BLASTX | 259 | 3.00E−22 | 62 | (AL162971) ribosomal protein L35-like |
| | | | | | | | [Arabidopsis thaliana] |
| 417 | LIB3602-043-Q6-K1-E8 | 7413650 | BLASTX | 245 | 6.00E−21 | 62 | (AL162971) ribosomal protein L35-like |
| | | | | | | | [Arabidopsis thaliana] |
| 418 | LIB3602-039-Q6-K1-B1 | 7413650 | BLASTX | 241 | 2.00E−20 | 62 | (AL162971) ribosomal protein L35-like |
| | | | | | | | [Arabidopsis thaliana] |
| 419 | LIB3602-114-Q1-K1-A4 | 7413650 | BLASTX | 231 | 4.00E−19 | 59 | (AL162971) ribosomal protein L35-like |
| | | | | | | | [Arabidopsis thaliana] |
| 420 | LIB3602-043-Q6-K1-H6 | 7413650 | BLASTX | 223 | 3.00E−18 | 60 | (AL162971) ribosomal protein L35-like |
| | | | | | | | [Arabidopsis thaliana] |
| 421 | LIB3602-022-Q6-K1-B4 | 7413650 | BLASTX | 219 | 6.00E−18 | 60 | (AL162971) ribosomal protein L35-like |
| | | | | | | | [Arabidopsis thaliana] |
| 422 | LIB3602-119-Q1-K1-A4 | 7413650 | BLASTX | 143 | 3.00E−09 | 68 | (AL162971) ribosomal protein L35-like |
| | | | | | | | [Arabidopsis thaliana] |
| 423 | LIB3602-105-Q1-K1-H2 | 1350976 | BLASTX | 813 | 4.00E−87 | 78 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 424 | LIB3602-089-Q6-K6-D7 | 1350976 | BLASTX | 721 | 3.00E−76 | 80 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 425 | LIB3602-118-Q1-K1-G8 | 1350976 | BLASTX | 515 | 2.00E−70 | 78 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 426 | LIB3602-012-Q6-K1-G12 | 1350976 | BLASTX | 633 | 5.00E−66 | 77 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 427 | LIB3602-112-Q1-K1-E9 | 1350976 | BLASTX | 539 | 6.00E−55 | 78 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 428 | LIB3602-003-Q1-K1-G12 | 1350976 | BLASTX | 507 | 3.00E−51 | 77 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 429 | LIB3602-120-Q1-K1-F12 | 1350976 | BLASTX | 325 | 2.00E−50 | 74 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 430 | LIB3602-029-Q6-K1-H11 | 1350976 | BLASTX | 491 | 2.00E−49 | 76 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 431 | LIB3602-028-Q6-K1-G9 | 1350976 | BLASTX | 418 | 8.00E−41 | 75 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 432 | LIB3602-101-Q1-K1-C8 | 1350976 | BLASTX | 413 | 2.00E−40 | 75 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 433 | LIB3602-017-Q6-K1-B7 | 1350976 | BLASTX | 409 | 9.00E−40 | 75 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 434 | LIB3602-004-Q1-K1-H8 | 1350976 | BLASTX | 293 | 3.00E−26 | 72 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 435 | LIB3602-046-Q6-K1-E5 | 1350976 | BLASTX | 278 | 2.00E−24 | 71 | 40S RIBOSOMAL PROTEIN S2 |
| | | | | | | | >gi\|939718\|gb\|AAA74091.1\| (U30454) ribosomal |
| | | | | | | | protein S2 [Urechis caupo] |
| 436 | LIB3602-020-Q6-K1-D12 | 1350976 | BLASTX | 257 | 4.00E−22 | 72 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 437 | LIB3602-003-Q1-K1-C12 | 1350976 | BLASTX | 245 | 1.00E−20 | 74 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 438 | LIB3602-111-Q1-K1-B7 | 1350976 | BLASTX | 243 | 2.00E−20 | 74 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 439 | LIB3602-021-Q6-K1-C7 | 1350976 | BLASTX | 172 | 2.00E−12 | 75 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 440 | LIB3602-032-Q6-K1-E7 | 1350976 | BLASTX | 155 | 3.00E−10 | 75 | 40S RIBOSOMAL PROTEIN S2 |
| | | | | | | | >gi\|939718\|gb\|AAA74095.1\| (U30454) ribosomal |
| | | | | | | | protein S2 [Urechis caupo] |
| 441 | LIB3602-059-Q6-K1-C5 | 400991 | BLASTX | 450 | 1.00E−44 | 66 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 442 | LIB3602-044-Q6-K1-A10 | 400991 | BLASTX | 450 | 1.00E−44 | 66 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 443 | LIB3602-041-Q6-K1-F10 | 400991 | BLASTX | 450 | 1.00E−44 | 66 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 444 | LIB3602-077-Q6-K6-H3 | 400991 | BLASTX | 450 | 1.00E−44 | 66 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 445 | LIB3602-078-Q6-K6-D10 | 400991 | BLASTX | 450 | 1.00E−44 | 66 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 446 | LIB3602-010-Q6-K1-C8 | 400991 | BLASTX | 450 | 1.00E−44 | 66 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 447 | LIB3602-029-Q6-K1-D9 | 400991 | BLASTX | 450 | 1.00E−44 | 66 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 448 | LIB3602-068-Q1-K1-C5 | 400991 | BLASTX | 450 | 2.00E−44 | 66 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 449 | LIB3602-053-Q6-K1-B9 | 400991 | BLASTX | 444 | 7.00E−44 | 66 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 450 | LIB3602-067-Q1-K1-A7 | 400991 | BLASTX | 428 | 5.00E−42 | 64 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 451 | LIB3602-022-Q6-K1-G9 | 400991 | BLASTX | 426 | 7.00E−42 | 64 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 452 | LIB3602-100-Q1-K1-C9 | 400991 | BLASTX | 412 | 4.00E−40 | 63 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 453 | LIB3602-038-Q6-K1-H9 | 400991 | BLASTX | 408 | 7.00E−40 | 70 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 454 | LIB3602-101-Q1-K1-B2 | 400991 | BLASTX | 287 | 5.00E−39 | 71 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 455 | LIB3602-071-Q1-K1-C7 | 400991 | BLASTX | 209 | 2.00E−30 | 58 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 456 | LIB3602-101-Q1-K1-H3 | 400991 | BLASTX | 235 | 1.00E−19 | 64 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 457 | LIB3602-086-Q6-K1-E7 | 400991 | BLASTX | 231 | 8.00E−19 | 45 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 458 | LIB3602-006-Q1-K1-E9 | 400991 | BLASTX | 224 | 2.00E−18 | 55 | 60S RIBOSOMAL PROTEIN L27 |
| | | | | | | | >gi\|282737\|pir\|\|S26612 ribosomal protein L27.e, |
| | | | | | | | cytosolic - green alga (Pyrobotrys stellata) |
| | | | | | | | >gi\|18267\|emb\|CAA48289.1\| (X68202) ribosomal |
| | | | | | | | protein L27 [Pyrobotrys stellata] |
| 459 | LIB3602-083-Q6-K6-A8 | 6939227 | BLASTX | 384 | 7.00E−37 | 66 | (AC020579) putative ribosomal protein |
| | | | | | | | [Arabidopsis thaliana] |
| 460 | LIB3602-070-Q1-K1-H2 | 6939227 | BLASTX | 384 | 7.00E−37 | 66 | (AC020579) putative ribosomal protein |
| | | | | | | | [Arabidopsis thaliana] |
| 461 | LIB3602-029-Q6-K1-E5 | 6939227 | BLASTX | 384 | 7.00E−37 | 66 | (AC020579) putative ribosomal protein |
| | | | | | | | [Arabidopsis thaliana] |
| 462 | LIB3602-058-Q6-K1-C8 | 6939227 | BLASTX | 384 | 7.00E−37 | 66 | (AC020579) putative ribosomal protein |
| | | | | | | | [Arabidopsis thaliana] |
| 463 | LIB3602-016-Q6-K1-A11 | 6939227 | BLASTX | 384 | 7.00E−37 | 66 | (AC020579) putative ribosomal protein |
| | | | | | | | [Arabidopsis thaliana] |
| 464 | LIB3602-012-Q6-K1-G11 | 6939227 | BLASTX | 384 | 7.00E−37 | 66 | (AC020579) putative ribosomal protein |
| | | | | | | | [Arabidopsis thaliana] |
| 465 | LIB3602-019-Q6-K1-H1 | 6939227 | BLASTX | 382 | 7.00E−37 | 64 | (AC020579) putative ribosomal protein |
| | | | | | | | [Arabidopsis thaliana] |
| 466 | LIB3602-065-Q1-K6-D10 | 6939227 | BLASTX | 384 | 1.00E−36 | 66 | (AC020579) putative ribosomal protein |
| | | | | | | | [Arabidopsis thaliana] |
| 467 | LIB3602-018-Q6-K1-H7 | 6939227 | BLASTX | 375 | 7.00E−36 | 65 | (AC020579) putative ribosomal protein |
| | | | | | | | [Arabidopsis thaliana] |
| 468 | LIB3602-109-Q1-K1-A8 | 6939227 | BLASTX | 363 | 2.00E−34 | 61 | (AC020579) putative ribosomal protein |
| | | | | | | | [Arabidopsis thaliana] |
| 469 | LIB3602-039-Q6-K1-B11 | 6939227 | BLASTX | 348 | 1.00E−32 | 63 | (AC020579) putative ribosomal protein |
| | | | | | | | [Arabidopsis thaliana] |
| 470 | LIB3602-100-Q1-K1-H2 | 6939227 | BLASTX | 284 | 3.00E−25 | 53 | (AC020579) putative ribosomal protein |
| | | | | | | | [Arabidopsis thaliana] |
| 471 | LIB3602-058-Q6-K1-B9 | 6939227 | BLASTX | 207 | 2.00E−16 | 64 | (AC020579) putative ribosomal protein |
| | | | | | | | [Arabidopsis thaliana] |
| 472 | LIB3602-091-Q6-K6-G11 | 6939227 | BLASTX | 169 | 3.00E−15 | 54 | (AC020579) putative ribosomal protein |
| | | | | | | | [Arabidopsis thaliana] |
| 473 | LIB3602-115-Q1-K1-B6 | 6939227 | BLASTX | 169 | 3.00E−15 | 54 | (AC020579) putative ribosomal protein |
| | | | | | | | [Arabidopsis thaliana] |
| 474 | LIB3602-088-Q6-K6-B6 | 6939227 | BLASTX | 169 | 3.00E−15 | 54 | (AC020579) putative ribosomal protein |
| | | | | | | | [Arabidopsis thaliana] |
| 475 | LIB3602-052-Q6-K1-D2 | 6939227 | BLASTX | 185 | 6.00E−14 | 64 | (AC020579) putative ribosomal protein |
| | | | | | | | [Arabidopsis thaliana] |
| 476 | LIB3602-057-Q6-K1-F11 | 6939227 | BLASTX | 184 | 1.00E−13 | 64 | (AC020579) putative ribosomal protein |
| | | | | | | | [Arabidopsis thaliana] |
| 477 | LIB3602-013-Q6-K1-C10 | 1710521 | BLASTX | 405 | 2.00E−39 | 63 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 478 | LIB3602-027-Q6-K1-F6 | 1710521 | BLASTX | 393 | 4.00E−38 | 62 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 479 | LIB3602-013-Q6-K1-D2 | 1710521 | BLASTX | 367 | 7.00E−35 | 60 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 480 | LIB3602-025-Q6-K1-G7 | 1710521 | BLASTX | 367 | 7.00E−35 | 60 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 481 | LIB3602-001-P1-K6-B9 | 1710521 | BLASTX | 367 | 8.00E−35 | 60 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 482 | LIB3602-001-P1-K6-D12 | 1710521 | BLASTX | 367 | 8.00E−35 | 60 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 483 | LIB3602-056-Q6-K1-F11 | 1710521 | BLASTX | 361 | 3.00E−34 | 59 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 484 | LIB3602-081-Q6-K6-A4 | 1710521 | BLASTX | 353 | 4.00E−33 | 59 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 485 | LIB3602-019-Q6-K1-G2 | 1710521 | BLASTX | 351 | 5.00E−33 | 58 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 486 | LIB3602-112-Q1-K1-B8 | 1710521 | BLASTX | 328 | 3.00E−30 | 57 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 487 | LIB3602-100-Q1-K1-E9 | 1710521 | BLASTX | 220 | 2.00E−29 | 58 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 488 | LIB3602-045-Q6-K1-B7 | 1710521 | BLASTX | 258 | 3.00E−22 | 56 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 489 | LIB3602-030-Q6-K1-F12 | 1710521 | BLASTX | 258 | 4.00E−22 | 56 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 490 | LIB3602-004-Q1-K1-G8 | 1710521 | BLASTX | 235 | 2.00E−19 | 56 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 491 | LIB3602-108-Q1-K1-C2 | 1710521 | BLASTX | 173 | 3.00E−12 | 57 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 492 | LIB3602-019-Q6-K1-G3 | 1710521 | BLASTX | 161 | 3.00E−11 | 44 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 493 | LIB3602-028-Q6-K1-C6 | 1710521 | BLASTX | 161 | 5.00E−11 | 56 | 60S RIBOSOMAL PROTEIN L24 |
| | | | | | | | >gi\|7440778\|pir\|\|T06178 ribosomal protein L24 - |
| | | | | | | | barley >gi\|1154859\|emb\|CAA63960.1\| (X94296) |
| | | | | | | | L24 ribosomal protein [Hordeum vulgare] |
| 494 | LIB3602-043-Q6-K1-G10 | 5902584 | BLASTX | 494 | 8.00E−50 | 71 | (AF110780) oxygen-evolving enhancer protein 1 |
| | | | | | | | precursor [Volvox carteri f. nagariensis] |
| 495 | LIB3602-060-Q6-K1-H10 | 5902584 | BLASTX | 467 | 1.00E−46 | 72 | (AF110780) oxygen-evolving enhancer protein 1 |
| | | | | | | | precursor [Volvox carteri f. nagariensis] |
| 496 | LIB3602-054-Q6-K1-B1 | 5902584 | BLASTX | 427 | 5.00E−42 | 78 | (AF110780) oxygen-evolving enhancer protein 1 |
| | | | | | | | precursor [Volvox carteri f. nagariensis] |
| 497 | LIB3602-014-Q6-K1-D12 | 5902584 | BLASTX | 419 | 4.00E−41 | 79 | (AF110780) oxygen-evolving enhancer protein 1 |
| | | | | | | | precursor [Volvox carteri f. nagariensis] |
| 498 | LIB3602-080-Q6-K6-F4 | 5902584 | BLASTX | 354 | 6.00E−37 | 77 | (AF110780) oxygen-evolving enhancer protein 1 |
| | | | | | | | precursor [Volvox carteri f. nagariensis] |
| 499 | LIB3602-058-Q6-K1-H6 | 5902584 | BLASTX | 375 | 6.00E−36 | 78 | (AF110780) oxygen-evolving enhancer protein 1 |
| | | | | | | | precursor [Volvox carteri f. nagariensis] |
| 500 | LIB3602-065-Q1-K6-F2 | 5902584 | BLASTX | 368 | 4.00E−35 | 76 | (AF110780) oxygen-evolving enhancer protein 1 |
| | | | | | | | precursor [Volvox carteri f. nagariensis] |
| 501 | LIB3602-092-Q6-K6-F12 | 5902584 | BLASTX | 354 | 4.00E−34 | 77 | (AF110780) oxygen-evolving enhancer protein 1 |
| | | | | | | | precursor [Volvox carteri f. nagariensis] |
| 502 | LIB3602-079-Q6-K6-F9 | 5902584 | BLASTX | 354 | 1.00E−33 | 77 | (AF110780) oxygen-evolving enhancer protein 1 |
| | | | | | | | precursor [Volvox carteri f. nagariensis] |
| 503 | LIB3602-088-Q6-K6-F3 | 5902584 | BLASTX | 350 | 7.00E−33 | 75 | (AF110780) oxygen-evolving enhancer protein 1 |
| | | | | | | | precursor [Volvox carteri f. nagariensis] |
| 504 | LIB3602-104-Q1-K1-E8 | 5902584 | BLASTX | 347 | 1.00E−32 | 76 | (AF110780) oxygen-evolving enhancer protein 1 |
| | | | | | | | precursor [Volvox carteri f. nagariensis] |
| 505 | LIB3602-064-Q1-K6-H7 | 5902584 | BLASTX | 346 | 1.00E−32 | 77 | (AF110780) oxygen-evolving enhancer protein 1 |
| | | | | | | | precursor [Volvox carteri f. nagariensis] |
| 506 | LIB3602-103-Q1-K1-E2 | 5902584 | BLASTX | 225 | 2.00E−32 | 82 | (AF110780) oxygen-evolving enhancer protein 1 |
| | | | | | | | precursor [Volvox carteri f. nagariensis] |
| 507 | LIB3602-093-Q6-K6-D7 | 5902584 | BLASTX | 271 | 7.00E−29 | 71 | (AF110780) oxygen-evolving enhancer protein 1 |
| | | | | | | | precursor [Volvox carteri f. nagariensis] |
| 508 | LIB3602-050-Q6-K1-A10 | 5902584 | BLASTX | 286 | 2.00E−25 | 76 | (AF110780) oxygen-evolving enhancer protein 1 |
| | | | | | | | precursor [Volvox carteri f. nagariensis] |
| 509 | LIB3602-077-Q6-K6-H7 | 5902584 | BLASTX | 253 | 7.00E−22 | 78 | (AF110780) oxygen-evolving enhancer protein 1 |
| | | | | | | | precursor [Volvox carteri f. nagariensis] |
| 510 | LIB3602-036-Q6-K1-F9 | 5902584 | BLASTX | 159 | 6.00E−11 | 87 | (AF110780) oxygen-evolving enhancer protein 1 |
| | | | | | | | precursor [Volvox carteri f. nagariensis] |
| 511 | LIB3602-030-Q6-K1-G9 | 6831602 | BLASTX | 408 | 8.00E−40 | 83 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 512 | LIB3602-014-Q6-K1-H8 | 6831602 | BLASTX | 408 | 8.00E−40 | 83 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 513 | LIB3602-034-Q6-K1-E1 | 6831602 | BLASTX | 408 | 8.00E−40 | 83 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 514 | LIB3602-017-Q6-K1-F11 | 6831602 | BLASTX | 387 | 2.00E−37 | 83 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 515 | LIB3602-027-Q6-K1-H10 | 6831602 | BLASTX | 382 | 7.00E−37 | 83 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 516 | LIB3602-035-Q1-K1-E1 | 6831602 | BLASTX | 378 | 3.00E−36 | 82 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 517 | LIB3602-069-Q1-K1-C12 | 6831602 | BLASTX | 378 | 3.00E−36 | 82 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 518 | LIB3602-087-Q6-K1-E1 | 6831602 | BLASTX | 378 | 3.00E−36 | 82 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 519 | LIB3602-021-Q6-K1-D6 | 6831602 | BLASTX | 371 | 1.00E−35 | 84 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 520 | LIB3602-021-Q6-K1-B12 | 6831602 | BLASTX | 369 | 2.00E−35 | 81 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 521 | LIB3602-019-Q6-K1-A12 | 6831602 | BLASTX | 365 | 7.00E−35 | 80 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 522 | LIB3602-021-Q6-K1-F9 | 6831602 | BLASTX | 327 | 1.00E−30 | 81 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 523 | LIB3602-067-Q1-K1-D5 | 6831602 | BLASTX | 182 | 1.00E−25 | 92 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 524 | LIB3602-054-Q6-K1-F9 | 6831602 | BLASTX | 275 | 2.00E−24 | 79 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 525 | LIB3602-060-Q6-K1-D12 | 6831602 | BLASTX | 239 | 3.00E−20 | 78 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 526 | LIB3602-043-Q6-K1-B11 | 6831602 | BLASTX | 207 | 8.00E−17 | 64 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 527 | LIB3602-011-Q6-K1-A6 | 6831602 | BLASTX | 204 | 8.00E−16 | 53 | 60S RIBOSOMAL PROTEIN L37A |
| | | | | | | | >gi\|4741896\|gb\|AAD28753.1\|AF127042_1 |
| | | | | | | | (AF127042) 60S ribosomal protein L37a |
| | | | | | | | [Gossypium hirsutum] |
| 528 | LIB3602-102-Q1-K1-H6 | 6174959 | BLASTX | 441 | 3.00E−50 | 71 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 529 | LIB3602-056-Q6-K1-B4 | 6174959 | BLASTX | 488 | 4.00E−49 | 69 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 530 | LIB3602-113-Q1-K1-B5 | 6174959 | BLASTX | 488 | 6.00E−49 | 70 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 531 | LIB3602-070-Q1-K1-F5 | 6174959 | BLASTX | 484 | 2.00E−48 | 70 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 532 | LIB3602-093-Q6-K6-E5 | 6174959 | BLASTX | 484 | 2.00E−48 | 70 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 533 | LIB3602-012-Q6-K1-C7 | 6174959 | BLASTX | 484 | 2.00E−48 | 70 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 534 | LIB3602-112-Q1-K1-B5 | 6174959 | BLASTX | 484 | 2.00E−48 | 70 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 535 | LIB3602-116-Q1-K1-B6 | 6174959 | BLASTX | 470 | 4.00E−47 | 71 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 536 | LIB3602-002-P1-K6-G8 | 6174959 | BLASTX | 452 | 8.00E−45 | 72 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 537 | LIB3602-059-Q6-K1-D12 | 6174959 | BLASTX | 331 | 3.00E−41 | 65 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 538 | LIB3602-038-Q6-K1-G10 | 6174959 | BLASTX | 416 | 1.00E−40 | 73 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 539 | LIB3602-042-Q6-K1-G2 | 6174959 | BLASTX | 408 | 1.00E−39 | 68 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 540 | LIB3602-120-Q1-K1-B5 | 6174959 | BLASTX | 292 | 1.00E−35 | 68 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 541 | LIB3602-114-Q1-K1-A1 | 6174959 | BLASTX | 350 | 4.00E−33 | 66 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 542 | LIB3602-072-Q1-K1-E9 | 6174959 | BLASTX | 274 | 3.00E−32 | 74 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 543 | LIB3602-006-Q1-K1-F11 | 6174959 | BLASTX | 199 | 4.00E−15 | 41 | 60S RIBOSOMAL PROTEIN L9 |
| | | | | | | | >gi\|7440702\|pir\|\|T03761 probable ribosomal protein |
| | | | | | | | L9 - rice >gi\|2058273\|dbj\|BAA19798.1\| (D83527) |
| | | | | | | | YK426 [Oryza sativa] |
| 544 | LIB3602-027-Q6-K1-H11 | 7440314 | BLASTX | 425 | 1.00E−41 | 60 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 545 | LIB3602-089-Q6-K6-E9 | 7440314 | BLASTX | 425 | 1.00E−41 | 60 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 546 | LIB3602-079-Q6-K6-A6 | 7440314 | BLASTX | 422 | 3.00E−41 | 60 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 547 | LIB3602-109-Q1-K1-B1 | 7440314 | BLASTX | 419 | 6.00E−41 | 61 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 548 | LIB3602-009-Q6-K1-F5 | 7440314 | BLASTX | 409 | 8.00E−40 | 75 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 549 | LIB3602-043-Q6-K1-B10 | 7440314 | BLASTX | 409 | 8.00E−40 | 75 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 550 | LIB3602-037-Q6-K1-D12 | 7440314 | BLASTX | 407 | 1.00E−39 | 74 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 551 | LIB3602-100-Q1-K1-D6 | 7440314 | BLASTX | 404 | 3.00E−39 | 74 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 552 | LIB3602-064-Q1-K6-C9 | 7440314 | BLASTX | 361 | 4.00E−36 | 58 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 553 | LIB3602-072-Q1-K1-G11 | 7440314 | BLASTX | 356 | 5.00E−36 | 57 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 554 | LIB3602-101-Q1-K1-C1 | 7440314 | BLASTX | 323 | 1.00E−35 | 71 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 555 | LIB3602-083-Q6-K6-A5 | 7440314 | BLASTX | 336 | 3.00E−31 | 56 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 556 | LIB3602-084-Q6-K1-B6 | 7440314 | BLASTX | 330 | 2.00E−30 | 54 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 557 | LIB3602-109-Q1-K1-A5 | 7440314 | BLASTX | 195 | 7.00E−24 | 76 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 558 | LIB3602-064-Q1-K6-F3 | 7440314 | BLASTX | 197 | 4.00E−18 | 44 | ribosomal protein S10, cytosolic - Arabidopsis |
| | | | | | | | thaliana >gi\|4539292\|emb\|CAB39595.1\| |
| | | | | | | | (AL049480) putative ribosomal protein S10 |
| | | | | | | | [Arabidopsis thaliana] |
| | | | | | | | >gi\|7269424\|emb\|CAB81384.1\| (AL161563) |
| | | | | | | | putative ribosomal protein S10 [Arabidopsis |
| | | | | | | | thaliana] |
| 559 | LIB3602-008-Q6-K1-C11 | 6683481 | BLASTX | 566 | 3.00E−58 | 81 | (AB018560) wrp15a [Citrullus lanatus] |
| 560 | LIB3602-025-Q6-K1-C10 | 6683481 | BLASTX | 566 | 3.00E−58 | 81 | (AB018560) wrp15a [Citrullus lanatus] |
| 561 | LIB3602-069-Q1-K1-B4 | 6683481 | BLASTX | 438 | 7.00E−52 | 79 | (AB018560) wrp15a [Citrullus lanatus] |
| 562 | LIB3602-068-Q1-K1-B4 | 6683481 | BLASTX | 438 | 8.00E−52 | 79 | (AB018560) wrp15a [Citrullus lanatus] |
| 563 | LIB3602-103-Q1-K1-C2 | 6683481 | BLASTX | 510 | 1.00E−51 | 81 | (AB018560) wrp15a [Citrullus lanatus] |
| 564 | LIB3602-105-Q1-K1-B5 | 6683481 | BLASTX | 438 | 8.00E−51 | 79 | (AB018560) wrp15a [Citrullus lanatus] |
| 565 | LIB3602-084-Q6-K1-B2 | 6683481 | BLASTX | 438 | 9.00E−50 | 79 | (AB018560) wrp15a [Citrullus lanatus] |
| 566 | LIB3602-047-Q6-K1-D6 | 6683481 | BLASTX | 443 | 8.00E−44 | 82 | (AB018560) wrp15a [Citrullus lanatus] |
| 567 | LIB3602-077-Q6-K6-H5 | 6683481 | BLASTX | 438 | 3.00E−43 | 79 | (AB018560) wrp15a [Citrullus lanatus] |
| 568 | LIB3602-055-Q6-K1-E12 | 6683481 | BLASTX | 428 | 4.00E−42 | 82 | (AB018560) wrp15a [Citrullus lanatus] |
| 569 | LIB3602-071-Q1-K1-F3 | 6683481 | BLASTX | 419 | 5.00E−41 | 79 | (AB018560) wrp15a [Citrullus lanatus] |
| 570 | LIB3602-074-Q1-K1-E7 | 6683481 | BLASTX | 341 | 4.00E−35 | 75 | (AB018560) wrp15a [Citrullus lanatus] |
| 571 | LIB3602-086-Q6-K1-H6 | 6683481 | BLASTX | 258 | 2.00E−32 | 66 | (AB018560) wrp15a [Citrullus lanatus] |
| 572 | LIB3602-077-Q6-K6-D5 | 6683481 | BLASTX | 319 | 2.00E−29 | 70 | (AB018560) wrp15a [Citrullus lanatus] |
| 573 | LIB3602-023-Q6-K1-G5 | 115778 | BLASTX | 653 | 2.00E−68 | 72 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 574 | LIB3602-094-Q6-K6-F3 | 115778 | BLASTX | 653 | 3.00E−68 | 71 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 575 | LIB3602-015-Q6-K1-G9 | 115778 | BLASTX | 536 | 1.00E−54 | 70 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 576 | LIB3602-008-Q6-K1-A6 | 115778 | BLASTX | 495 | 7.00E−50 | 70 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 577 | LIB3602-013-Q6-K1-H5 | 115778 | BLASTX | 478 | 8.00E−48 | 70 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 578 | LIB3602-090-Q6-K6-B4 | 115778 | BLASTX | 471 | 6.00E−47 | 71 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 579 | LIB3602-105-Q1-K1-F2 | 115778 | BLASTX | 464 | 4.00E−46 | 71 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 580 | LIB3602-025-Q6-K1-H12 | 115778 | BLASTX | 459 | 1.00E−45 | 71 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 581 | LIB3602-105-Q1-K1-B7 | 115778 | BLASTX | 431 | 2.00E−42 | 70 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 582 | LIB3602-029-Q6-K1-B7 | 115778 | BLASTX | 409 | 9.00E−40 | 70 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 583 | LIB3602-050-Q6-K1-B9 | 115778 | BLASTX | 392 | 7.00E−38 | 71 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 584 | LIB3602-027-Q6-K1-A7 | 115778 | BLASTX | 364 | 2.00E−34 | 61 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 585 | LIB3602-018-Q6-K1-A1 | 115778 | BLASTX | 319 | 2.00E−33 | 59 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 586 | LIB3602-115-Q1-K1-D2 | 115778 | BLASTX | 151 | 1.00E−13 | 63 | CHLOROPHYLL A-B BINDING PROTEIN 1 |
| | | | | | | | PRECURSOR (LHCII TYPE I CAB-1) (LHCP) |
| | | | | | | | >gi\|282896\|pir\|\|S22511 chlorophyll a/b-binding |
| | | | | | | | protein precursor - white mustard |
| | | | | | | | >gi\|21138\|emb\|CAA34459.1\| (X16436) chlorophyll |
| | | | | | | | a/b-binding protein (AA 1-266) [Sinapis alba] |
| | | | | | | | >gi\|21140 |
| 587 | LIB3602-103-Q1-K1-C4 | 548852 | BLASTX | 303 | 2.00E−27 | 65 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 588 | LIB3602-037-Q6-K1-C5 | 548852 | BLASTX | 303 | 2.00E−27 | 65 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 589 | LIB3602-053-Q6-K1-C7 | 548852 | BLASTX | 303 | 2.00E−27 | 65 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 590 | LIB3602-066-Q1-K6-C8 | 548852 | BLASTX | 303 | 2.00E−27 | 65 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 591 | LIB3602-030-Q6-K1-G1 | 548852 | BLASTX | 303 | 2.00E−27 | 65 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 592 | LIB3602-063-Q1-K6-D8 | 548852 | BLASTX | 298 | 7.00E−27 | 65 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 593 | LIB3602-093-Q6-K6-A11 | 548852 | BLASTX | 298 | 1.00E−26 | 65 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 594 | LIB3602-034-Q6-K1-D2 | 548852 | BLASTX | 291 | 4.00E−26 | 65 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 595 | LIB3602-104-Q1-K1-A11 | 548852 | BLASTX | 286 | 3.00E−25 | 61 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 596 | LIB3602-048-Q6-K1QA-H5 | 548852 | BLASTX | 257 | 4.00E−22 | 66 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 597 | LIB3602-016-Q6-K1-H5 | 548852 | BLASTX | 254 | 9.00E−22 | 65 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 598 | LIB3602-115-Q1-K1-H12 | 548852 | BLASTX | 229 | 1.00E−21 | 60 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 599 | LIB3602-073-Q1-K1-G7 | 548852 | BLASTX | 240 | 4.00E−20 | 63 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 600 | LIB3602-101-Q1-K1-C4 | 548852 | BLASTX | 145 | 6.00E−20 | 69 | 40S RIBOSOMAL PROTEIN S21 |
| | | | | | | | >gi\|481227\|pir\|\|S38357 ribosomal protein S21, |
| | | | | | | | cytosolic - rice >gi\|303839\|dbj\|BAA02158.1\| |
| | | | | | | | (D12633) 40S subunit ribosomal protein [Oryza |
| | | | | | | | sativa] |
| 601 | LIB3602-115-Q1-K1-D5 | 115832 | BLASTX | 264 | 5.00E−23 | 87 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 602 | LIB3602-095-Q6-K6-F1 | 115832 | BLASTX | 266 | 6.00E−23 | 73 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 603 | LIB3602-009-Q6-K1-F11 | 115832 | BLASTX | 261 | 2.00E−22 | 75 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 604 | LIB3602-009-Q6-K1-E8 | 115832 | BLASTX | 261 | 2.00E−22 | 75 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 605 | LIB3602-034-Q6-K1-A4 | 115832 | BLASTX | 258 | 3.00E−22 | 87 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 606 | LIB3602-063-Q1-K6-E1 | 115832 | BLASTX | 258 | 5.00E−22 | 69 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 607 | LIB3602-035-Q1-K1-A4 | 115832 | BLASTX | 244 | 2.00E−20 | 86 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 608 | LIB3602-061-Q6-K1-A5 | 115832 | BLASTX | 205 | 6.00E−16 | 71 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 609 | LIB3602-025-Q6-K1-B2 | 115832 | BLASTX | 205 | 7.00E−16 | 71 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 610 | LIB3602-053-Q6-K1-A4 | 115832 | BLASTX | 178 | 6.00E−13 | 86 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 611 | LIB3602-018-Q6-K1-G6 | 115832 | BLASTX | 174 | 1.00E−12 | 86 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 612 | LIB3602-006-Q1-K1-B9 | 115832 | BLASTX | 145 | 2.00E−10 | 62 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 613 | LIB3602-082-Q6-K6-E4 | 115832 | BLASTX | 143 | 7.00E−09 | 65 | CHLOROPHYLL A-B BINDING PROTEIN |
| | | | | | | | PRECURSOR (LHCII TYPE I CAB) (LHCP) |
| | | | | | | | >gi\|81414\|pir\|\|JS0171 chlorophyll a/b-binding |
| | | | | | | | protein precursor - moss (Physcomitrella patens) |
| | | | | | | | >gi\|169020\|gb\|AAA33636.1\| (M23532) major |
| | | | | | | | chlorophyll binding protein [Physcomitrella patens] |
| 614 | LIB3602-073-Q1-K1-G12 | 4262180 | BLASTX | 823 | 3.00E−88 | 86 | (AC005508) 29621 [Arabidopsis thaliana] |
| 615 | LIB3602-113-Q1-K1-H1 | 4262180 | BLASTX | 822 | 4.00E−88 | 84 | (AC005508) 29621 [Arabidopsis thaliana] |
| 616 | LIB3602-106-Q1-K1-A5 | 4262180 | BLASTX | 807 | 2.00E−86 | 84 | (AC005508) 29621 [Arabidopsis thaliana] |
| 617 | LIB3602-007-Q1-K1-F8 | 4262180 | BLASTX | 791 | 2.00E−84 | 86 | (AC005508) 29621 [Arabidopsis thaliana] |
| 618 | LIB3602-070-Q1-K1-B1 | 4262180 | BLASTX | 732 | 1.00E−77 | 80 | (AC005508) 29621 [Arabidopsis thaliana] |
| 619 | LIB3602-007-Q1-K1-B10 | 4262180 | BLASTX | 701 | 5.00E−74 | 88 | (AC005508) 29621 [Arabidopsis thaliana] |
| 620 | LIB3602-032-Q6-K1-E1 | 4262180 | BLASTX | 701 | 5.00E−74 | 88 | (AC005508) 29621 [Arabidopsis thaliana] |
| 621 | LIB3602-047-Q6-K1-D2 | 4262180 | BLASTX | 701 | 5.00E−74 | 88 | (AC005508) 29621 [Arabidopsis thaliana] |
| 622 | LIB3602-111-Q1-K1-G4 | 4262180 | BLASTX | 543 | 1.00E−68 | 86 | (AC005508) 29621 [Arabidopsis thaliana] |
| 623 | LIB3602-054-Q6-K1-B7 | 4262180 | BLASTX | 648 | 7.00E−68 | 81 | (AC005508) 29621 [Arabidopsis thaliana] |
| 624 | LIB3602-102-Q1-K1-D8 | 4262180 | BLASTX | 507 | 8.00E−65 | 87 | (AC005508) 29621 [Arabidopsis thaliana] |
| 625 | LIB3602-120-Q1-K1-H1 | 4262180 | BLASTX | 514 | 3.00E−52 | 87 | (AC005508) 29621 [Arabidopsis thaliana] |
| 626 | LIB3602-119-Q1-K1-B12 | 4262180 | BLASTX | 288 | 1.00E−43 | 81 | (AC005508) 29621 [Arabidopsis thaliana] |
| 627 | LIB3602-102-Q1-K1-F9 | 6984222 | BLASTX | 572 | 6.00E−59 | 75 | (AF227979) 40S ribosomal protein S16 [Euphorbia |
| | | | | | | | esula] |
| 628 | LIB3602-054-Q6-K1-H4 | 6984222 | BLASTX | 572 | 6.00E−59 | 75 | (AF227979) 40S ribosomal protein S16 [Euphorbia |
| | | | | | | | esula] |
| 629 | LIB3602-018-Q6-K1-E4 | 6984222 | BLASTX | 572 | 7.00E−59 | 75 | (AF227979) 40S ribosomal protein S16 [Euphorbia |
| | | | | | | | esula] |
| 630 | LIB3602-074-Q1-K1-B4 | 6984222 | BLASTX | 572 | 7.00E−59 | 75 | (AF227979) 40S ribosomal protein S16 [Euphorbia |
| | | | | | | | esula] |
| 631 | LIB3602-023-Q6-K1-D4 | 6984222 | BLASTX | 572 | 7.00E−59 | 75 | (AF227979) 40S ribosomal protein S16 [Euphorbia |
| | | | | | | | esula] |
| 632 | LIB3602-051-Q6-K1-E1 | 6984222 | BLASTX | 568 | 2.00E−58 | 74 | (AF227979) 40S ribosomal protein S16 [Euphorbia |
| | | | | | | | esula] |
| 633 | LIB3602-007-Q1-K1-F2 | 6984222 | BLASTX | 544 | 2.00E−55 | 72 | (AF227979) 40S ribosomal protein S16 [Euphorbia |
| | | | | | | | esula] |
| 634 | LIB3602-069-Q1-K1-C5 | 6984222 | BLASTX | 523 | 6.00E−54 | 73 | (AF227979) 40S ribosomal protein S16 [Euphorbia |
| | | | | | | | esula] |
| 635 | LIB3602-070-Q1-K1-D10 | 6984222 | BLASTX | 523 | 6.00E−54 | 73 | (AF227979) 40S ribosomal protein S16 [Euphorbia |
| | | | | | | | esula] |
| 636 | LIB3602-116-Q1-K1-E12 | 6984222 | BLASTX | 431 | 1.00E−53 | 79 | (AF227979) 40S ribosomal protein S16 [Euphorbia |
| | | | | | | | esula] |
| 637 | LIB3602-034-Q6-K1-B1 | 6984222 | BLASTX | 510 | 1.00E−51 | 69 | (AF227979) 40S ribosomal protein S16 [Euphorbia |
| | | | | | | | esula] |
| 638 | LIB3602-034-Q6-K1-A1 | 6984222 | BLASTX | 225 | 1.00E−18 | 59 | (AF227979) 40S ribosomal protein S16 [Euphorbia |
| | | | | | | | esula] |
| 639 | LIB3602-068-Q1-K1-C7 | 6984222 | BLASTX | 198 | 2.00E−15 | 86 | (AF227979) 40S ribosomal protein S16 [Euphorbia |
| | | | | | | | esula] |
| 640 | LIB3602-104-Q1-K1-E1 | 2440160 | BLASTX | 279 | 2.00E−24 | 50 | (Y14836) beta-galactosidase [Phagemid cloning |
| | | | | | | | vector pTZ19U] |
| 641 | LIB3602-112-Q1-K1-D6 | 2440160 | BLASTX | 274 | 8.00E−24 | 53 | (Y14836) beta-galactosidase [Phagemid cloning |
| | | | | | | | vector pTZ19U] |
| 642 | LIB3602-061-Q6-K1-H10 | 2440160 | BLASTX | 242 | 3.00E−20 | 56 | (Y14836) beta-galactosidase [Phagemid cloning |
| | | | | | | | vector pTZ19U] |
| 643 | LIB3602-094-Q6-K6-C4 | 2440160 | BLASTX | 219 | 2.00E−17 | 41 | (Y14836) beta-galactosidase [Phagemid cloning |
| | | | | | | | vector pTZ19U] |
| 644 | LIB3602-004-Q1-K1-F8 | 2440160 | BLASTX | 216 | 4.00E−17 | 43 | (Y14836) beta-galactosidase [Phagemid cloning |
| | | | | | | | vector pTZ19U] |
| 645 | LIB3602-076-Q6-K6-F12 | 2440160 | BLASTX | 200 | 4.00E−15 | 40 | (Y14836) beta-galactosidase [Phagemid cloning |
| | | | | | | | vector pTZ19U] |
| 646 | LIB3602-041-Q6-K1-B7 | 2440160 | BLASTX | 161 | 5.00E−15 | 55 | (Y14836) beta-galactosidase [Phagemid cloning |
| | | | | | | | vector pTZ19U] |
| 647 | LIB3602-006-Q1-K1-A5 | 2440160 | BLASTX | 191 | 3.00E−14 | 41 | (Y14836) beta-galactosidase [Phagemid cloning |
| | | | | | | | vector pTZ19U] |
| 648 | LIB3602-001-P1-K6-D9 | 2440160 | BLASTX | 177 | 1.00E−12 | 36 | (Y14836) beta-galactosidase [Phagemid cloning |
| | | | | | | | vector pTZ19U] |
| 649 | LIB3602-115-Q1-K1-F9 | 2440160 | BLASTX | 172 | 4.00E−12 | 36 | (Y14836) beta-galactosidase [Phagemid cloning |
| | | | | | | | vector pTZ19U] |
| 650 | LIB3602-036-Q6-K1-E11 | 2440160 | BLASTX | 152 | 1.00E−09 | 36 | (Y14836) beta-galactosidase [Phagemid cloning |
| | | | | | | | vector pTZ19U] |
| 651 | LIB3602-062-Q6-K1-A5 | 2440160 | BLASTX | 150 | 2.00E−09 | 36 | (Y14836) beta-galactosidase [Phagemid cloning |
| | | | | | | | vector pTZ19U] |
| 652 | LIB3602-052-Q6-K1-B3 | 2440160 | BLASTX | 144 | 9.00E−09 | 38 | (Y14836) beta-galactosidase [Phagemid cloning |
| | | | | | | | vector pTZ19U] |
| 653 | LIB3602-018-Q6-K1-E8 | 7440313 | BLASTX | 611 | 2.00E−63 | 73 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 654 | LIB3602-104-Q1-K1-H4 | 7440313 | BLASTX | 611 | 2.00E−63 | 68 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 655 | LIB3602-073-Q1-K1-H12 | 7440313 | BLASTX | 435 | 4.00E−53 | 65 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 656 | LIB3602-107-Q1-K1-H5 | 7440313 | BLASTX | 475 | 2.00E−47 | 64 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 657 | LIB3602-115-Q1-K1-B8 | 7440313 | BLASTX | 463 | 5.00E−46 | 56 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 658 | LIB3602-004-Q1-K1-G1 | 7440313 | BLASTX | 451 | 7.00E−45 | 51 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 659 | LIB3602-053-Q6-K1-C5 | 7440313 | BLASTX | 417 | 9.00E−41 | 62 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 660 | LIB3602-001-P1-K6-E9 | 7440313 | BLASTX | 409 | 9.00E−40 | 62 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 661 | LIB3602-020-Q6-K1-E3 | 7440313 | BLASTX | 386 | 4.00E−37 | 60 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 662 | LIB3602-012-Q6-K1-E10 | 7440313 | BLASTX | 234 | 2.00E−19 | 53 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 663 | LIB3602-079-Q6-K6-H5 | 7440313 | BLASTX | 159 | 8.00E−17 | 65 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 664 | LIB3602-060-Q6-K1-F7 | 7440313 | BLASTX | 154 | 4.00E−10 | 64 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 665 | LIB3602-119-Q1-K1-G3 | 7440313 | BLASTX | 149 | 2.00E−09 | 77 | ribosomal protein S6.1, cytosolic - maize |
| | | | | | | | >gi\|1917019\|gb\|AAB51304.1\| (U92045) ribosomal |
| | | | | | | | protein S6 RPS6-1 [Zea mays] |
| 666 | LIB3602-026-Q6-K1-F6 | 7293042 | BLASTX | 371 | 1.00E−35 | 80 | (AE003498) CG9091 gene product [Drosophila |
| | | | | | | | melanogaster] |
| 667 | LIB3602-068-Q1-K1-D10 | 7293042 | BLASTX | 371 | 2.00E−35 | 80 | (AE003498) CG9091 gene product [Drosophila |
| | | | | | | | melanogaster] |
| 668 | LIB3602-077-Q6-K6-A6 | 7293042 | BLASTX | 371 | 2.00E−35 | 80 | (AE003498) CG9091 gene product [Drosophila |
| | | | | | | | melanogaster] |
| 669 | LIB3602-079-Q6-K6-B2 | 7293042 | BLASTX | 371 | 2.00E−35 | 80 | (AE003498) CG9091 gene product [Drosophila |
| | | | | | | | melanogaster] |
| 670 | LIB3602-105-Q1-K1-D5 | 7293042 | BLASTX | 371 | 2.00E−35 | 80 | (AE003498) CG9091 gene product [Drosophila |
| | | | | | | | melanogaster] |
| 671 | LIB3602-068-Q1-K1-B9 | 7293042 | BLASTX | 371 | 2.00E−35 | 80 | (AE003498) CG9091 gene product [Drosophila |
| | | | | | | | melanogaster] |
| 672 | LIB3602-037-Q6-K1-F8 | 7293042 | BLASTX | 371 | 2.00E−35 | 80 | (AE003498) CG9091 gene product [Drosophila |
| | | | | | | | melanogaster] |
| 673 | LIB3602-027-Q6-K1-D1 | 7293042 | BLASTX | 371 | 2.00E−35 | 80 | (AE003498) CG9091 gene product [Drosophila |
| | | | | | | | melanogaster] |
| 674 | LIB3602-057-Q6-K1-E9 | 7293042 | BLASTX | 364 | 1.00E−34 | 79 | (AE003498) CG9091 gene product [Drosophila |
| | | | | | | | melanogaster] |
| 675 | LIB3602-053-Q6-K1-F11 | 7293042 | BLASTX | 351 | 4.00E−33 | 80 | (AE003498) CG9091 gene product [Drosophila |
| | | | | | | | melanogaster] |
| 676 | LIB3602-053-Q6-K1-F5 | 7293042 | BLASTX | 348 | 6.00E−33 | 79 | (AE003498) CG9091 gene product [Drosophila |
| | | | | | | | melanogaster] |
| 677 | LIB3602-106-Q1-K1-F5 | 7293042 | BLASTX | 245 | 2.00E−24 | 82 | (AE003498) CG9091 gene product [Drosophila |
| | | | | | | | melanogaster] |
| 678 | LIB3602-120-Q1-K1-E11 | 7293042 | BLASTX | 232 | 2.00E−19 | 80 | (AE003498) CG9091 gene product [Drosophila |
| | | | | | | | melanogaster] |
| 679 | LIB3602-055-Q6-K1-A6 | 445613 | BLASTX | 542 | 2.00E−55 | 75 | ribosomal protein L7 [Solanum tuberosum] |
| 680 | LIB3602-089-Q6-K6-E7 | 445613 | BLASTX | 542 | 3.00E−55 | 75 | ribosomal protein L7 [Solanum tuberosum] |
| 681 | LIB3602-057-Q6-K1-C4 | 445613 | BLASTX | 533 | 3.00E−54 | 73 | ribosomal protein L7 [Solanum tuberosum] |
| 682 | LIB3602-034-Q6-K1-D4 | 445613 | BLASTX | 532 | 3.00E−54 | 74 | ribosomal protein L7 [Solanum tuberosum] |
| 683 | LIB3602-059-Q6-K1-G10 | 445613 | BLASTX | 521 | 7.00E−53 | 73 | ribosomal protein L7 [Solanum tuberosum] |
| 684 | LIB3602-035-Q1-K1-D4 | 445613 | BLASTX | 497 | 4.00E−50 | 72 | ribosomal protein L7 [Solanum tuberosum] |
| 685 | LIB3602-070-Q1-K1-D9 | 445613 | BLASTX | 473 | 3.00E−47 | 62 | ribosomal protein L7 [Solanum tuberosum] |
| 686 | LIB3602-061-Q6-K1-F6 | 445613 | BLASTX | 442 | 7.00E−44 | 71 | ribosomal protein L7 [Solanum tuberosum] |
| 687 | LIB3602-040-Q6-K1-G8 | 445613 | BLASTX | 412 | 2.00E−40 | 71 | ribosomal protein L7 [Solanum tuberosum] |
| 688 | LIB3602-111-Q1-K1-A5 | 445613 | BLASTX | 236 | 8.00E−36 | 68 | ribosomal protein L7 [Solanum tuberosum] |
| 689 | LIB3602-040-Q6-K1-A3 | 445613 | BLASTX | 349 | 7.00E−33 | 70 | ribosomal protein L7 [Solanum tuberosum] |
| 690 | LIB3602-025-Q6-K1-B5 | 445613 | BLASTX | 339 | 1.00E−31 | 70 | ribosomal protein L7 [Solanum tuberosum] |
| 691 | LIB3602-018-Q6-K1-D9 | 445613 | BLASTX | 292 | 3.00E−26 | 69 | ribosomal protein L7 [Solanum tuberosum] |
| 692 | LIB3602-047-Q6-K1-B8 | 6441068 | BLASTX | 675 | 5.00E−71 | 77 | (AU066549) 60S ribosomal protein L4 |
| | | | | | | | [Chlamydomonas sp.] |
| 693 | LIB3602-072-Q1-K1-B7 | 6441068 | BLASTX | 572 | 8.00E−59 | 59 | (AU066549) 60S ribosomal protein L4 |
| | | | | | | | [Chlamydomonas sp.] |
| 694 | LIB3602-061-Q6-K1-A1 | 6441068 | BLASTX | 564 | 5.00E−58 | 71 | (AU066549) 60S ribosomal protein L4 |
| | | | | | | | [Chlamydomonas sp.] |
| 695 | LIB3602-113-Q1-K1-G4 | 6441068 | BLASTX | 531 | 5.00E−54 | 59 | (AU066549) 60S ribosomal protein L4 |
| | | | | | | | [Chlamydomonas sp.] |
| 696 | LIB3602-059-Q6-K1-A5 | 6441068 | BLASTX | 507 | 3.00E−51 | 70 | (AU066549) 60S ribosomal protein L4 |
| | | | | | | | [Chlamydomonas sp.] |
| 697 | LIB3602-063-Q1-K6-D4 | 6441068 | BLASTX | 505 | 4.00E−51 | 70 | (AU066549) 60S ribosomal protein L4 |
| | | | | | | | [Chlamydomonas sp.] |
| 698 | LIB3602-046-Q6-K1-B7 | 6441068 | BLASTX | 473 | 2.00E−47 | 71 | (AU066549) 60S ribosomal protein L4 |
| | | | | | | | [Chlamydomonas sp.] |
| 699 | LIB3602-046-Q6-K1-E3 | 6441068 | BLASTX | 335 | 3.00E−31 | 54 | (AU066549) 60S ribosomal protein L4 |
| | | | | | | | [Chlamydomonas sp.] |
| 700 | LIB3602-059-Q6-K1-A6 | 6441068 | BLASTX | 202 | 3.00E−30 | 60 | (AU066549) 60S ribosomal protein L4 |
| | | | | | | | [Chlamydomonas sp.] |
| 701 | LIB3602-107-Q1-K1-H11 | 6441068 | BLASTX | 297 | 1.00E−26 | 48 | (AU066549) 60S ribosomal protein L4 |
| | | | | | | | [Chlamydomonas sp.] |
| 702 | LIB3602-093-Q6-K6-D10 | 6441068 | BLASTX | 292 | 1.00E−26 | 73 | (AU066549) 60S ribosomal protein L4 |
| | | | | | | | [Chlamydomonas sp.] |
| 703 | LIB3602-020-Q6-K1-B6 | 6441068 | BLASTX | 177 | 1.00E−24 | 69 | (AU066549) 60S ribosomal protein L4 |
| | | | | | | | [Chlamydomonas sp.] |
| 704 | LIB3602-057-Q6-K1-F4 | 6441068 | BLASTX | 166 | 2.00E−22 | 40 | (AU066549) 60S ribosomal protein L4 |
| | | | | | | | [Chlamydomonas sp.] |
| 705 | LIB3602-071-Q1-K1-D6 | 6320978 | BLASTX | 276 | 4.00E−24 | 59 | Ribosomal protein S26B; Rps26bp |
| | | | | | | | >gi\|730459\|sp\|P39939\|R26B_YEAST 40S |
| | | | | | | | RIBOSOMAL PROTEIN S26-B |
| | | | | | | | >gi\|1071981\|pir\|\|S50634 ribosomal protein S26.e.B, |
| | | | | | | | cytosolic - yeast (Saccharomyces cerevisiae) |
| | | | | | | | >gi\|603370\|gb\|AAC03229.1\| (U18916) Rps26bp |
| | | | | | | | [Saccharomyces cerevisia |
| 706 | LIB3602-106-Q1-K1-D11 | 6320978 | BLASTX | 276 | 4.00E−24 | 59 | Ribosomal protein S26B; Rps26bp |
| | | | | | | | >gi\|730459\|sp\|P39939\|R26B_YEAST 40S |
| | | | | | | | RIBOSOMAL PROTEIN S26-B |
| | | | | | | | >gi\|1071981\|pir\|\|S50634 ribosomal protein S26.e.B, |
| | | | | | | | cytosolic - yeast (Saccharomyces cerevisiae) |
| | | | | | | | >gi\|603370\|gb\|AAC03229.1\| (U18916) Rps26bp |
| | | | | | | | [Saccharomyces cerevisia |
| 707 | LIB3602-067-Q1-K1-D10 | 6320978 | BLASTX | 276 | 4.00E−24 | 59 | Ribosomal protein S26B; Rps26bp |
| | | | | | | | >gi\|730459\|sp\|P39939\|R26B_YEAST 40S |
| | | | | | | | RIBOSOMAL PROTEIN S26-B |
| | | | | | | | >gi\|1071981\|pir\|\|S50634 ribosomal protein S26.e.B, |
| | | | | | | | cytosolic - yeast (Saccharomyces cerevisiae) |
| | | | | | | | >gi\|603370\|gb\|AAC03229.1\| (U18916) Rps26bp |
| | | | | | | | [Saccharomyces cerevisia |
| 708 | LIB3602-084-Q6-K1-A12 | 6320978 | BLASTX | 271 | 1.00E−23 | 59 | Ribosomal protein S26B; Rps26bp |
| | | | | | | | >gi\|730459\|sp\|P39939\|R26B_YEAST 40S |
| | | | | | | | RIBOSOMAL PROTEIN S26-B |
| | | | | | | | >gi\|1071981\|pir\|\|S50634 ribosomal protein S26.e.B, |
| | | | | | | | cytosolic - yeast (Saccharomyces cerevisiae) |
| | | | | | | | >gi\|603370\|gb\|AAC03229.1\| (U18916) Rps26bp |
| | | | | | | | [Saccharomyces cerevisia |
| 709 | LIB3602-053-Q6-K1-A2 | 6320978 | BLASTX | 269 | 1.00E−23 | 59 | Ribosomal protein S26B; Rps26bp |
| | | | | | | | >gi\|730459\|sp\|P39939\|R26B_YEAST 40S |
| | | | | | | | RIBOSOMAL PROTEIN S26-B |
| | | | | | | | >gi\|1071981\|pir\|\|S50634 ribosomal protein S26.e.B, |
| | | | | | | | cytosolic - yeast (Saccharomyces cerevisiae) |
| | | | | | | | >gi\|603370\|gb\|AAC03229.1\| (U18916) Rps26bp |
| | | | | | | | [Saccharomyces cerevisia |
| 710 | LIB3602-027-Q6-K1-H8 | 6320978 | BLASTX | 269 | 2.00E−23 | 59 | Ribosomal protein S26B; Rps26bp |
| | | | | | | | >gi\|730459\|sp\|P39939\|R26B_YEAST 40S |
| | | | | | | | RIBOSOMAL PROTEIN S26-B |
| | | | | | | | >gi\|1071981\|pir\|\|S50634 ribosomal protein S26.e.B, |
| | | | | | | | cytosolic - yeast (Saccharomyces cerevisiae) |
| | | | | | | | >gi\|603370\|gb\|AAC03229.1\| (U18916) Rps26bp |
| | | | | | | | [Saccharomyces cerevisia |
| 711 | LIB3602-043-Q6-K1-E5 | 6320978 | BLASTX | 269 | 2.00E−23 | 59 | Ribosomal protein S26B; Rps26bp |
| | | | | | | | >gi\|730459\|sp\|P39939\|R26B_YEAST 40S |
| | | | | | | | RIBOSOMAL PROTEIN S26-B |
| | | | | | | | >gi\|1071981\|pir\|\|S50634 ribosomal protein S26.e.B, |
| | | | | | | | cytosolic - yeast (Saccharomyces cerevisiae) |
| | | | | | | | >gi\|603370\|gb\|AAC03229.1\| (U18916) Rps26bp |
| | | | | | | | [Saccharomyces cerevisia |
| 712 | LIB3602-041-Q6-K1-C3 | 6320978 | BLASTX | 269 | 3.00E−23 | 59 | Ribosomal protein S26B; Rps26bp |
| | | | | | | | >gi\|730459\|sp\|P39939\|R26B_YEAST 40S |
| | | | | | | | RIBOSOMAL PROTEIN S26-B |
| | | | | | | | >gi\|1071981\|pir\|\|S50634 ribosomal protein S26.e.B, |
| | | | | | | | cytosolic - yeast (Saccharomyces cerevisiae) |
| | | | | | | | >gi\|603370\|gb\|AAC03229.1\| (U18916) Rps26bp |
| | | | | | | | [Saccharomyces cerevisia |
| 713 | LIB3602-017-Q6-K1-D12 | 6320978 | BLASTX | 262 | 2.00E−22 | 58 | Ribosomal protein S26B; Rps26bp |
| | | | | | | | >gi\|730459\|sp\|P39939\|R26B_YEAST 40S |
| | | | | | | | RIBOSOMAL PROTEIN S26-B |
| | | | | | | | >gi\|1071981\|pir\|\|S50634 ribosomal protein S26.e.B, |
| | | | | | | | cytosolic - yeast (Saccharomyces cerevisiae) |
| | | | | | | | >gi\|603370\|gb\|AAC03229.1\| (U18916) Rps26bp |
| | | | | | | | [Saccharomyces cerevisia |
| 714 | LIB3602-029-Q6-K1-A2 | 6320978 | BLASTX | 260 | 3.00E−22 | 57 | Ribosomal protein S26B; Rps26bp |
| | | | | | | | >gi\|730459\|sp\|P39939\|R26B_YEAST 40S |
| | | | | | | | RIBOSOMAL PROTEIN S26-B |
| | | | | | | | >gi\|1071981\|pir\|\|S50634 ribosomal protein S26.e.B, |
| | | | | | | | cytosolic - yeast (Saccharomyces cerevisiae) |
| | | | | | | | >gi\|603370\|gb\|AAC03229.1\| (U18916) Rps26bp |
| | | | | | | | [Saccharomyces cerevisia |
| 715 | LIB3602-111-Q1-K1-A3 | 6320978 | BLASTX | 212 | 4.00E−19 | 58 | Ribosomal protein S26B; Rps26bp |
| | | | | | | | >gi\|730459\|sp\|P39939\|R26B_YEAST 40S |
| | | | | | | | RIBOSOMAL PROTEIN S26-B |
| | | | | | | | >gi\|1071981\|pir\|\|S50634 ribosomal protein S26.e.B, |
| | | | | | | | cytosolic - yeast (Saccharomyces cerevisiae) |
| | | | | | | | >gi\|603370\|gb\|AAC03229.1\| (U18916) Rps26bp |
| | | | | | | | [Saccharomyces cerevisia |
| 716 | LIB3602-107-Q1-K1-H9 | 6320978 | BLASTX | 195 | 7.00E−15 | 59 | Ribosomal protein S26B; Rps26bp |
| | | | | | | | >gi\|730459\|sp\|P39939\|R26B_YEAST 40S |
| | | | | | | | RIBOSOMAL PROTEIN S26-B |
| | | | | | | | >gi\|1071981\|pir\|\|S50634 ribosomal protein S26.e.B, |
| | | | | | | | cytosolic - yeast (Saccharomyces cerevisiae) |
| | | | | | | | >gi\|603370\|gb\|AAC03229.1\| (U18916) Rps26bp |
| | | | | | | | [Saccharomyces cerevisia |
| 717 | LIB3602-090-Q6-K6-H12 | 3122703 | BLASTX | 485 | 1.00E−48 | 75 | 60S RIBOSOMAL PROTEIN L23A |
| | | | | | | | >gi\|2641201\|gb\|AAB86852.1\| (AF031542) |
| | | | | | | | ribosomal protein L23a [Fritillaria agrestis] |
| 718 | LIB3602-045-Q6-K1-D4 | 3122703 | BLASTX | 479 | 5.00E−48 | 73 | 60S RIBOSOMAL PROTEIN L23A |
| | | | | | | | >gi\|2641201\|gb\|AAB86852.1\| (AF031542) |
| | | | | | | | ribosomal protein L23a [Fritillaria agrestis] |
| 719 | LIB3602-091-Q6-K6-H12 | 3122703 | BLASTX | 479 | 6.00E−48 | 74 | 60S RIBOSOMAL PROTEIN L23A |
| | | | | | | | >gi\|2641201\|gb\|AAB86852.1\| (AF031542) |
| | | | | | | | ribosomal protein L23a [Fritillaria agrestis] |
| 720 | LIB3602-055-Q6-K1-D4 | 3122703 | BLASTX | 473 | 2.00E−47 | 75 | 60S RIBOSOMAL PROTEIN L23A |
| | | | | | | | >gi\|2641201\|gb\|AAB86852.1\| (AF031542) |
| | | | | | | | ribosomal protein L23a [Fritillaria agrestis] |
| 721 | LIB3602-094-Q6-K6-G1 | 3122703 | BLASTX | 473 | 3.00E−47 | 75 | 60S RIBOSOMAL PROTEIN L23A |
| | | | | | | | >gi\|2641201\|gb\|AAB86852.1\| (AF031542) |
| | | | | | | | ribosomal protein L23a [Fritillaria agrestis] |
| 722 | LIB3602-115-Q1-K1-E5 | 3122703 | BLASTX | 473 | 3.00E−47 | 75 | 60S RIBOSOMAL PROTEIN L23A |
| | | | | | | | >gi\|2641201\|gb\|AAB86852.1\| (AF031542) |
| | | | | | | | ribosomal protein L23a [Fritillaria agrestis] |
| 723 | LIB3602-114-Q1-K1-H1 | 3122703 | BLASTX | 473 | 3.00E−47 | 75 | 60S RIBOSOMAL PROTEIN L23A |
| | | | | | | | >gi\|2641201\|gb\|AAB86852.1\| (AF031542) |
| | | | | | | | ribosomal protein L23a [Fritillaria agrestis] |
| 724 | LIB3602-029-Q6-K1-D4 | 3122703 | BLASTX | 473 | 3.00E−47 | 75 | 60S RIBOSOMAL PROTEIN L23A |
| | | | | | | | >gi\|2641201\|gb\|AAB86852.1\| (AF031542) |
| | | | | | | | ribosomal protein L23a [Fritillaria agrestis] |
| 725 | LIB3602-091-Q6-K6-G1 | 3122703 | BLASTX | 473 | 3.00E−47 | 75 | 60S RIBOSOMAL PROTEIN L23A |
| | | | | | | | >gi\|2641201\|gb\|AAB86852.1\| (AF031542) |
| | | | | | | | ribosomal protein L23a [Fritillaria agrestis] |
| 726 | LIB3602-111-Q1-K1-H2 | 3122703 | BLASTX | 456 | 3.00E−45 | 76 | 60S RIBOSOMAL PROTEIN L23A |
| | | | | | | | >gi\|2641201\|gb\|AAB86852.1\| (AF031542) |
| | | | | | | | ribosomal protein L23a [Fritillaria agrestis] |
| 727 | LIB3602-108-Q1-K1-C10 | 3122703 | BLASTX | 439 | 1.00E−44 | 75 | 60S RIBOSOMAL PROTEIN L23A |
| | | | | | | | >gi\|2641201\|gb\|AAB86852.1\| (AF031542) |
| | | | | | | | ribosomal protein L23a [Fritillaria agrestis] |
| 728 | LIB3602-093-Q6-K6-F1 | 3122703 | BLASTX | 201 | 4.00E−21 | 47 | 60S RIBOSOMAL PROTEIN L23A |
| | | | | | | | >gi\|2641201\|gb\|AAB86852.1\| (AF031542) |
| | | | | | | | ribosomal protein L23a [Fritillaria agrestis] |
| 729 | LIB3602-060-Q6-K1-C10 | 1173222 | BLASTX | 565 | 4.00E−58 | 71 | 40S RIBOSOMAL PROTEIN S11 |
| | | | | | | | >gi\|7440474\|pir\|\|T10730 ribosomal protein S11 - |
| | | | | | | | green alga (Dunaliella tertiolecta) |
| | | | | | | | >gi\|534959\|emb\|CAA46835.1\| (X66036) ribosomal |
| | | | | | | | protein S11 [Dunaliella tertiolecta] |
| 730 | LIB3602-094-Q6-K6-H2 | 1173222 | BLASTX | 519 | 5.00E−54 | 78 | 40S RIBOSOMAL PROTEIN S11 |
| | | | | | | | >gi\|7440474\|pir\|\|T10730 ribosomal protein S11 - |
| | | | | | | | green alga (Dunaliella tertiolecta) |
| | | | | | | | >gi\|534959\|emb\|CAA46835.1\| (X66036) ribosomal |
| | | | | | | | protein S11 [Dunaliella tertiolecta] |
| 731 | LIB3602-104-Q1-K1-H7 | 1173222 | BLASTX | 519 | 1.00E−52 | 78 | 40S RIBOSOMAL PROTEIN S11 |
| | | | | | | | >gi\|7440474\|pir\|\|T10730 ribosomal protein S11 - |
| | | | | | | | green alga (Dunaliella tertiolecta) |
| | | | | | | | >gi\|534959\|emb\|CAA46835.1\| (X66036) ribosomal |
| | | | | | | | protein S11 [Dunaliella tertiolecta] |
| 732 | LIB3602-111-Q1-K1-H9 | 1173222 | BLASTX | 516 | 2.00E−52 | 76 | 40S RIBOSOMAL PROTEIN S11 |
| | | | | | | | >gi\|7440474\|pir\|\|T10730 ribosomal protein S11 - |
| | | | | | | | green alga (Dunaliella tertiolecta) |
| | | | | | | | >gi\|534959\|emb\|CAA46835.1\| (X66036) ribosomal |
| | | | | | | | protein S11 [Dunaliella tertiolecta] |
| 733 | LIB3602-088-Q6-K6-G4 | 1173222 | BLASTX | 445 | 1.00E−47 | 81 | 40S RIBOSOMAL PROTEIN S11 |
| | | | | | | | >gi\|7440474\|pir\|\|T10730 ribosomal protein S11 - |
| | | | | | | | green alga (Dunaliella tertiolecta) |
| | | | | | | | >gi\|534959\|emb\|CAA46835.1\| (X66036) ribosomal |
| | | | | | | | protein S11 [Dunaliella tertiolecta] |
| 734 | LIB3602-114-Q1-K1-E2 | 1173222 | BLASTX | 446 | 5.00E−44 | 80 | 40S RIBOSOMAL PROTEIN S11 |
| | | | | | | | >gi\|7440474\|pir\|\|T10730 ribosomal protein S11 - |
| | | | | | | | green alga (Dunaliella tertiolecta) |
| | | | | | | | >gi\|534959\|emb\|CAA46835.1\| (X66036) ribosomal |
| | | | | | | | protein S11 [Dunaliella tertiolecta] |
| 735 | LIB3602-088-Q6-K6-C11 | 1173222 | BLASTX | 445 | 6.00E−44 | 80 | 40S RIBOSOMAL PROTEIN S11 |
| | | | | | | | >gi\|7440474\|pir\|\|T10730 ribosomal protein S11 - |
| | | | | | | | green alga (Dunaliella tertiolecta) |
| | | | | | | | >gi\|534959\|emb\|CAA46835.1\| (X66036) ribosomal |
| | | | | | | | protein S11 [Dunaliella tertiolecta] |
| 736 | LIB3602-108-Q1-K1-E7 | 1173222 | BLASTX | 421 | 7.00E−43 | 75 | 40S RIBOSOMAL PROTEIN S11 |
| | | | | | | | >gi\|7440474\|pir\|\|T10730 ribosomal protein S11 - |
| | | | | | | | green alga (Dunaliella tertiolecta) |
| | | | | | | | >gi\|534959\|emb\|CAA46835.1\| (X66036) ribosomal |
| | | | | | | | protein S11 [Dunaliella tertiolecta] |
| 737 | LIB3602-060-Q6-K1-E9 | 1173222 | BLASTX | 418 | 5.00E−41 | 68 | 40S RIBOSOMAL PROTEIN S11 |
| | | | | | | | >gi\|7440474\|pir\|\|T10730 ribosomal protein S11 - |
| | | | | | | | green alga (Dunaliella tertiolecta) |
| | | | | | | | >gi\|534959\|emb\|CAA46835.1\| (X66036) ribosomal |
| | | | | | | | protein S11 [Dunaliella tertiolecta] |
| 738 | LIB3602-090-Q6-K6-H4 | 1173222 | BLASTX | 278 | 2.00E−40 | 83 | 40S RIBOSOMAL PROTEIN S11 |
| | | | | | | | >gi\|7440474\|pir\|\|T10730 ribosomal protein S11 - |
| | | | | | | | green alga (Dunaliella tertiolecta) |
| | | | | | | | >gi\|534959\|emb\|CAA46835.1\| (X66036) ribosomal |
| | | | | | | | protein S11 [Dunaliella tertiolecta] |
| 739 | LIB3602-077-Q6-K6-G4 | 1173222 | BLASTX | 311 | 3.00E−28 | 80 | 40S RIBOSOMAL PROTEIN S11 |
| | | | | | | | >gi\|7440474\|pir\|\|T10730 ribosomal protein S11 - |
| | | | | | | | green alga (Dunaliella tertiolecta) |
| | | | | | | | >gi\|534959\|emb\|CAA46835.1\| (X66036) ribosomal |
| | | | | | | | protein S11 [Dunaliella tertiolecta] |
| 740 | LIB3602-108-Q1-K1-F11 | 1173222 | BLASTX | 297 | 1.00E−27 | 62 | 40S RIBOSOMAL PROTEIN S11 |
| | | | | | | | >gi\|7440474\|pir\|\|T10730 ribosomal protein S11 - |
| | | | | | | | green alga (Dunaliella tertiolecta) |
| | | | | | | | >gi\|534959\|emb\|CAA46835.1\| (X66036) ribosomal |
| | | | | | | | protein S11 [Dunaliella tertiolecta] |
| 741 | LIB3602-043-Q6-K1-G12 | 4038471 | BLASTX | 313 | 1.00E−28 | 74 | (AF111029) 40S ribosomal protein S27 homolog |
| | | | | | | | [Zea mays] |
| 742 | LIB3602-011-Q6-K1-D1 | 4038471 | BLASTX | 313 | 2.00E−28 | 74 | (AF111029) 40S ribosomal protein S27 homolog |
| | | | | | | | [Zea mays] |
| 743 | LIB3602-062-Q6-K1-F6 | 4038471 | BLASTX | 313 | 2.00E−28 | 74 | (AF111029) 40S ribosomal protein S27 homolog |
| | | | | | | | [Zea mays] |
| 744 | LIB3602-020-Q6-K1-D6 | 4038471 | BLASTX | 303 | 2.00E−27 | 88 | (AF111029) 40S ribosomal protein S27 homolog |
| | | | | | | | [Zea mays] |
| 745 | LIB3602-055-Q6-K1-B10 | 4038471 | BLASTX | 298 | 8.00E−27 | 74 | (AF111029) 40S ribosomal protein S27 homolog |
| | | | | | | | [Zea mays] |
| 746 | LIB3602-032-Q6-K1-E12 | 4038471 | BLASTX | 287 | 1.00E−25 | 89 | (AF111029) 40S ribosomal protein S27 homolog |
| | | | | | | | [Zea mays] |
| 747 | LIB3602-039-Q6-K1-H9 | 4038471 | BLASTX | 259 | 4.00E−22 | 90 | (AF111029) 40S ribosomal protein S27 homolog |
| | | | | | | | [Zea mays] |
| 748 | LIB3602-056-Q6-K1-C2 | 4038471 | BLASTX | 249 | 4.00E−21 | 89 | (AF111029) 40S ribosomal protein S27 homolog |
| | | | | | | | [Zea mays] |
| 749 | LIB3602-090-Q6-K6-E4 | 4038471 | BLASTX | 229 | 1.00E−18 | 91 | (AF111029) 40S ribosomal protein S27 homolog |
| | | | | | | | [Zea mays] |
| 750 | LIB3602-113-Q1-K1-G9 | 4038471 | BLASTX | 206 | 6.00E−16 | 90 | (AF111029) 40S ribosomal protein S27 homolog |
| | | | | | | | [Zea mays] |
| 751 | LIB3602-055-Q6-K1-B11 | 4038471 | BLASTX | 165 | 3.00E−11 | 64 | (AF111029) 40S ribosomal protein S27 homolog |
| | | | | | | | [Zea mays] |
| 752 | LIB3602-001-P1-K6-F4 | 4038471 | BLASTX | 156 | 4.00E−10 | 90 | (AF111029) 40S ribosomal protein S27 homolog |
| | | | | | | | [Zea mays] |
| 753 | LIB3602-060-Q6-K1-G5 | 115829 | BLASTX | 684 | 5.00E−72 | 78 | CHLOROPHYLL A-B BINDING PROTEIN OF |
| | | | | | | | LHCII TYPE I PRECURSOR (CAB) (LHCP) |
| | | | | | | | >gi\|99364\|pir\|\|JW0040 chlorophyll a/b-binding |
| | | | | | | | protein 28.5K precursor - green alga (Dunaliella |
| | | | | | | | tertiolecta) >gi\|167985\|gb\|AAA62772.1\| (M60049) |
| | | | | | | | 28.5 kDa LHCII apoprotein [Dunaliella ter |
| 754 | LIB3602-085-Q6-K1-C6 | 115829 | BLASTX | 635 | 3.00E−66 | 75 | CHLOROPHYLL A-B BINDING PROTEIN OF |
| | | | | | | | LHCII TYPE I PRECURSOR (CAB) (LHCP) |
| | | | | | | | >gi\|99364\|pir\|\|JW0040 chlorophyll a/b-binding |
| | | | | | | | protein 28.5K precursor - green alga (Dunaliella |
| | | | | | | | tertiolecta) >gi\|167985\|gb\|AAA62772.1\| (M60049) |
| | | | | | | | 28.5 kDa LHCII apoprotein [Dunaliella ter |
| 755 | LIB3602-054-Q6-K1-A8 | 115829 | BLASTX | 603 | 1.00E−62 | 77 | CHLOROPHYLL A-B BINDING PROTEIN OF |
| | | | | | | | LHCII TYPE I PRECURSOR (CAB) (LHCP) |
| | | | | | | | >gi\|99364\|pir\|\|JW0040 chlorophyll a/b-binding |
| | | | | | | | protein 28.5K precursor - green alga (Dunaliella |
| | | | | | | | tertiolecta) >gi\|167985\|gb\|AAA62772.1\| (M60049) |
| | | | | | | | 28.5 kDa LHCII apoprotein [Dunaliella ter |
| 756 | LIB3602-094-Q6-K6-A5 | 115829 | BLASTX | 547 | 5.00E−61 | 75 | CHLOROPHYLL A-B BINDING PROTEIN OF |
| | | | | | | | LHCII TYPE I PRECURSOR (CAB) (LHCP) |
| | | | | | | | >gi\|99364\|pir\|\|JW0040 chlorophyll a/b-binding |
| | | | | | | | protein 28.5K precursor - green alga (Dunaliella |
| | | | | | | | tertiolecta) >gi\|167985\|gb\|AAA62772.1\| (M60049) |
| | | | | | | | 28.5 kDa LHCII apoprotein [Dunaliella ter |
| 757 | LIB3602-018-Q6-K1-F7 | 115829 | BLASTX | 564 | 6.00E−58 | 64 | CHLOROPHYLL A-B BINDING PROTEIN OF |
| | | | | | | | LHCII TYPE I PRECURSOR (CAB) (LHCP) |
| | | | | | | | >gi\|99364\|pir\|\|JW0040 chlorophyll a/b-binding |
| | | | | | | | protein 28.5K precursor - green alga (Dunaliella |
| | | | | | | | tertiolecta) >gi\|167985\|gb\|AAA62772.1\| (M60049) |
| | | | | | | | 28.5 kDa LHCII apoprotein [Dunaliella ter |
| 758 | LIB3602-071-Q1-K1-D3 | 115829 | BLASTX | 558 | 4.00E−57 | 75 | CHLOROPHYLL A-B BINDING PROTEIN OF |
| | | | | | | | LHCII TYPE I PRECURSOR (CAB) (LHCP) |
| | | | | | | | >gi\|99364\|pir\|\|JW0040 chlorophyll a/b-binding |
| | | | | | | | protein 28.5K precursor - green alga (Dunaliella |
| | | | | | | | tertiolecta) >gi\|167985\|gb\|AAA62772.1\| (M60049) |
| | | | | | | | 28.5 kDa LHCII apoprotein [Dunaliella ter |
| 759 | LIB3602-002-P1-K6-D8 | 115829 | BLASTX | 451 | 1.00E−44 | 75 | CHLOROPHYLL A-B BINDING PROTEIN OF |
| | | | | | | | LHCII TYPE I PRECURSOR (CAB) (LHCP) |
| | | | | | | | >gi\|99364\|pir\|\|JW0040 chlorophyll a/b-binding |
| | | | | | | | protein 28.5K precursor - green alga (Dunaliella |
| | | | | | | | tertiolecta) >gi\|167985\|gb\|AAA62772.1\| (M60049) |
| | | | | | | | 28.5 kDa LHCII apoprotein [Dunaliella ter |
| 760 | LIB3602-051-Q6-K1-C2 | 115829 | BLASTX | 358 | 5.00E−42 | 74 | CHLOROPHYLL A-B BINDING PROTEIN OF |
| | | | | | | | LHCII TYPE I PRECURSOR (CAB) (LHCP) |
| | | | | | | | >gi\|99364\|pir\|\|JW0040 chlorophyll a/b-binding |
| | | | | | | | protein 28.5K precursor - green alga (Dunaliella |
| | | | | | | | tertiolecta) >gi\|167985\|gb\|AAA62772.1\| (M60049) |
| | | | | | | | 28.5 kDa LHCII apoprotein [Dunaliella ter |
| 761 | LIB3602-111-Q1-K1-E11 | 115829 | BLASTX | 297 | 7.00E−42 | 75 | CHLOROPHYLL A-B BINDING PROTEIN OF |
| | | | | | | | LHCII TYPE I PRECURSOR (CAB) (LHCP) |
| | | | | | | | >gi\|99364\|pir\|\|JW0040 chlorophyll a/b-binding |
| | | | | | | | protein 28.5K precursor - green alga (Dunaliella |
| | | | | | | | tertiolecta) >gi\|167985\|gb\|AAA62772.1\| (M60049) |
| | | | | | | | 28.5 kDa LHCII apoprotein [Dunaliella ter |
| 762 | LIB3602-090-Q6-K6-A12 | 115829 | BLASTX | 256 | 5.00E−33 | 67 | CHLOROPHYLL A-B BINDING PROTEIN OF |
| | | | | | | | LHCII TYPE I PRECURSOR (CAB) (LHCP) |
| | | | | | | | >gi\|99364\|pir\|\|JW0040 chlorophyll a/b-binding |
| | | | | | | | protein 28.5K precursor - green alga (Dunaliella |
| | | | | | | | tertiolecta) >gi\|167985\|gb\|AAA62772.1\| (M60049) |
| | | | | | | | 28.5 kDa LHCII apoprotein [Dunaliella ter |
| 763 | LIB3602-023-Q6-K1-H1 | 115829 | BLASTX | 275 | 3.00E−30 | 44 | CHLOROPHYLL A-B BINDING PROTEIN OF |
| | | | | | | | LHCII TYPE I PRECURSOR (CAB) (LHCP) |
| | | | | | | | >gi\|99364\|pir\|\|JW0040 chlorophyll a/b-binding |
| | | | | | | | protein 28.5K precursor - green alga (Dunaliella |
| | | | | | | | tertiolecta) >gi\|167985\|gb\|AAA62772.1\| (M60049) |
| | | | | | | | 28.5 kDa LHCII apoprotein [Dunaliella ter |
| 764 | LIB3602-001-P1-K6-D6 | 115829 | BLASTX | 315 | 5.00E−29 | 77 | CHLOROPHYLL A-B BINDING PROTEIN OF |
| | | | | | | | LHCII TYPE I PRECURSOR (CAB) (LHCP) |
| | | | | | | | >gi\|99364\|pir\|\|JW0040 chlorophyll a/b-binding |
| | | | | | | | protein 28.5K precursor - green alga (Dunaliella |
| | | | | | | | tertiolecta) >gi\|167985\|gb\|AAA62772.1\| (M60049) |
| | | | | | | | 28.5 kDa LHCII apoprotein [Dunaliella ter |
| 765 | LIB3602-042-Q6-K1-G9 | 1172977 | BLASTX | 716 | 1.00E−75 | 74 | 60S RIBOSOMAL PROTEIN L18 |
| | | | | | | | >gi\|606970\|gb\|AAA69928.1\| (U15741) cytoplasmic |
| | | | | | | | ribosomal protein L18 [Arabidopsis thaliana] |
| 766 | LIB3602-042-Q6-K1-G10 | 1172977 | BLASTX | 709 | 8.00E−75 | 74 | 60S RIBOSOMAL PROTEIN L18 |
| | | | | | | | >gi\|606970\|gb\|AAA69928.1\| (U15741) cytoplasmic |
| | | | | | | | ribosomal protein L18 [Arabidopsis thaliana] |
| 767 | LIB3602-067-Q1-K1-D6 | 1172977 | BLASTX | 694 | 4.00E−73 | 74 | 60S RIBOSOMAL PROTEIN L18 |
| | | | | | | | >gi\|606970\|gb\|AAA69928.1\| (U15741) cytoplasmic |
| | | | | | | | ribosomal protein L18 [Arabidopsis thaliana] |
| 768 | LIB3602-089-Q6-K6-B5 | 1172977 | BLASTX | 688 | 2.00E−72 | 73 | 60S RIBOSOMAL PROTEIN L18 |
| | | | | | | | >gi\|606970\|gb\|AAA69928.1\| (U15741) cytoplasmic |
| | | | | | | | ribosomal protein L18 [Arabidopsis thaliana] |
| 769 | LIB3602-084-Q6-K1-A10 | 1172977 | BLASTX | 673 | 1.00E−70 | 73 | 60S RIBOSOMAL PROTEIN L18 |
| | | | | | | | >gi\|606970\|gb\|AAA69928.1\| (U15741) cytoplasmic |
| | | | | | | | ribosomal protein L18 [Arabidopsis thaliana] |
| 770 | LIB3602-069-Q1-K1-D5 | 1172977 | BLASTX | 669 | 3.00E−70 | 76 | 60S RIBOSOMAL PROTEIN L18 |
| | | | | | | | >gi\|606970\|gb\|AAA69928.1\| (U15741) cytoplasmic |
| | | | | | | | ribosomal protein L18 [Arabidopsis thaliana] |
| 771 | LIB3602-068-Q1-K1-G4 | 1172977 | BLASTX | 638 | 2.00E−66 | 76 | 60S RIBOSOMAL PROTEIN L18 |
| | | | | | | | >gi\|606970\|gb\|AAA69928.1\| (U15741) cytoplasmic |
| | | | | | | | ribosomal protein L18 [Arabidopsis thaliana] |
| 772 | LIB3602-092-Q6-K6-E6 | 1172977 | BLASTX | 630 | 1.00E−65 | 73 | 60S RIBOSOMAL PROTEIN L18 |
| | | | | | | | >gi\|606970\|gb\|AAA69928.1\| (U15741) cytoplasmic |
| | | | | | | | ribosomal protein L18 [Arabidopsis thaliana] |
| 773 | LIB3602-003-Q1-K1-D12 | 1172977 | BLASTX | 573 | 5.00E−59 | 75 | 60S RIBOSOMAL PROTEIN L18 |
| | | | | | | | >gi\|606970\|gb\|AAA69928.1\| (U15741) cytoplasmic |
| | | | | | | | ribosomal protein L18 [Arabidopsis thaliana] |
| 774 | LIB3602-003-Q1-K1-G6 | 1172977 | BLASTX | 546 | 6.00E−56 | 75 | 60S RIBOSOMAL PROTEIN L18 |
| | | | | | | | >gi\|606970\|gb\|AAA69928.1\| (U15741) cytoplasmic |
| | | | | | | | ribosomal protein L18 [Arabidopsis thaliana] |
| 775 | LIB3602-057-Q6-K1-F7 | 1172977 | BLASTX | 439 | 6.00E−54 | 75 | 60S RIBOSOMAL PROTEIN L18 |
| | | | | | | | >gi\|606970\|gb\|AAA69928.1\| (U15741) cytoplasmic |
| | | | | | | | ribosomal protein L18 [Arabidopsis thaliana] |
| 776 | LIB3602-056-Q6-K1-C4 | 1172977 | BLASTX | 498 | 3.00E−50 | 70 | 60S RIBOSOMAL PROTEIN L18 |
| | | | | | | | >gi\|606970\|gb\|AAA69928.1\| (U15741) cytoplasmic |
| | | | | | | | ribosomal protein L18 [Arabidopsis thaliana] |
| 777 | LIB3602-052-Q6-K1-F1 | 1350717 | BLASTX | 376 | 5.00E−36 | 67 | 60S RIBOSOMAL PROTEIN L30 |
| | | | | | | | >gi\|422700\|pir\|\|S34608 ribosomal protein L30, |
| | | | | | | | cytosolic - chicken >gi\|402507\|dbj\|BAA03394.1\| |
| | | | | | | | (D14521) ribosomal protein L30 [Gallus gallus] |
| 778 | LIB3602-011-Q6-K1-H2 | 1350717 | BLASTX | 339 | 1.00E−31 | 61 | 60S RIBOSOMAL PROTEIN L30 |
| | | | | | | | >gi\|422700\|pir\|\|S34608 ribosomal protein L30, |
| | | | | | | | cytosolic - chicken >gi\|402507\|dbj\|BAA03394.1\| |
| | | | | | | | (D14521) ribosomal protein L30 [Gallus gallus] |
| 779 | LIB3602-066-Q1-K6-H12 | 1350717 | BLASTX | 339 | 1.00E−31 | 61 | 60S RIBOSOMAL PROTEIN L30 |
| | | | | | | | >gi\|422700\|pir\|\|S34608 ribosomal protein L30, |
| | | | | | | | cytosolic - chicken >gi\|402507\|dbj\|BAA03394.1\| |
| | | | | | | | (D14521) ribosomal protein L30 [Gallus gallus] |
| 780 | LIB3602-066-Q1-K6-C7 | 1350717 | BLASTX | 339 | 1.00E−31 | 61 | 60S RIBOSOMAL PROTEIN L30 |
| | | | | | | | >gi\|422700\|pir\|\|S34608 ribosomal protein L30, |
| | | | | | | | cytosolic - chicken >gi\|402507\|dbj\|BAA03394.1\| |
| | | | | | | | (D14521) ribosomal protein L30 [Gallus gallus] |
| 781 | LIB3602-108-Q1-K1-A9 | 1350717 | BLASTX | 336 | 2.00E−31 | 60 | 60S RIBOSOMAL PROTEIN L30 |
| | | | | | | | >gi\|422700\|pir\|\|S34608 ribosomal protein L30, |
| | | | | | | | cytosolic - chicken >gi\|402507\|dbj\|BAA03394.1\| |
| | | | | | | | (D14521) ribosomal protein L30 [Gallus gallus] |
| 782 | LIB3602-106-Q1-K1-D4 | 1350717 | BLASTX | 336 | 3.00E−31 | 65 | 60S RIBOSOMAL PROTEIN L30 |
| | | | | | | | >gi\|422700\|pir\|\|S34608 ribosomal protein L30, |
| | | | | | | | cytosolic - chicken >gi\|402507\|dbj\|BAA03394.1\| |
| | | | | | | | (D14521) ribosomal protein L30 [Gallus gallus] |
| 783 | LIB3602-071-Q1-K1-H12 | 1350717 | BLASTX | 336 | 3.00E−31 | 65 | 60S RIBOSOMAL PROTEIN L30 |
| | | | | | | | >gi\|422700\|pir\|\|S34608 ribosomal protein L30, |
| | | | | | | | cytosolic - chicken >gi\|402507\|dbj\|BAA03394.1\| |
| | | | | | | | (D14521) ribosomal protein L30 [Gallus gallus] |
| 784 | LIB3602-056-Q6-K1-F12 | 1350717 | BLASTX | 331 | 8.00E−31 | 59 | 60S RIBOSOMAL PROTEIN L30 |
| | | | | | | | >gi\|422700\|pir\|\|S34608 ribosomal protein L30, |
| | | | | | | | cytosolic - chicken >gi\|402507\|dbj\|BAA03394.1\| |
| | | | | | | | (D14521) ribosomal protein L30 [Gallus gallus] |
| 785 | LIB3602-004-Q1-K1-C10 | 1350717 | BLASTX | 327 | 2.00E−30 | 65 | 60S RIBOSOMAL PROTEIN L30 |
| | | | | | | | >gi\|422700\|pir\|\|S34608 ribosomal protein L30, |
| | | | | | | | cytosolic - chicken >gi\|402507\|dbj\|BAA03394.1\| |
| | | | | | | | (D14521) ribosomal protein L30 [Gallus gallus] |
| 786 | LIB3602-049-Q6-K1-E8 | 1350717 | BLASTX | 276 | 1.00E−24 | 73 | 60S RIBOSOMAL PROTEIN L30 |
| | | | | | | | >gi\|422700\|pir\|\|S34608 ribosomal protein L30, |
| | | | | | | | cytosolic - chicken >gi\|402507\|dbj\|BAA03394.1\| |
| | | | | | | | (D14521) ribosomal protein L30 [Gallus gallus] |
| 787 | LIB3602-006-Q1-K1-G5 | 1350717 | BLASTX | 177 | 8.00E−13 | 68 | 60S RIBOSOMAL PROTEIN L30 |
| | | | | | | | >gi\|422700\|pir\|\|S34608 ribosomal protein L30, |
| | | | | | | | cytosolic - chicken >gi\|402507\|dbj\|BAA03394.1\| |
| | | | | | | | (D14521) ribosomal protein L30 [Gallus gallus] |
| 788 | LIB3602-039-Q6-K1-G7 | 3717978 | BLASTX | 790 | 2.00E−84 | 80 | (Y12431) 5S ribosomal protein [Mus musculus] |
| 789 | LIB3602-041-Q6-K1-F5 | 3717978 | BLASTX | 767 | 1.00E−81 | 80 | (Y12431) 5S ribosomal protein [Mus musculus] |
| 790 | LIB3602-017-Q6-K1-C3 | 3717978 | BLASTX | 744 | 5.00E−79 | 81 | (Y12431) 5S ribosomal protein [Mus musculus] |
| 791 | LIB3602-003-Q1-K1-G8 | 3717978 | BLASTX | 733 | 1.00E−77 | 79 | (Y12431) 5S ribosomal protein [Mus musculus] |
| 792 | LIB3602-107-Q1-K1-A1 | 3717978 | BLASTX | 723 | 1.00E−76 | 78 | (Y12431) 5S ribosomal protein [Mus musculus] |
| 793 | LIB3602-028-Q6-K1-E1 | 3717978 | BLASTX | 695 | 2.00E−73 | 79 | (Y12431) 5S ribosomal protein [Mus musculus] |
| 794 | LIB3602-062-Q6-K1-C3 | 3717978 | BLASTX | 690 | 1.00E−72 | 78 | (Y12431) 5S ribosomal protein [Mus musculus] |
| 795 | LIB3602-048-Q6-K1QA-C2 | 3717978 | BLASTX | 667 | 5.00E−70 | 77 | (Y12431) 5S ribosomal protein [Mus musculus] |
| 796 | LIB3602-045-Q6-K1-H5 | 3717978 | BLASTX | 585 | 2.00E−60 | 76 | (Y12431) 5S ribosomal protein [Mus musculus] |
| 797 | LIB3602-108-Q1-K1-E8 | 3717978 | BLASTX | 418 | 4.00E−51 | 77 | (Y12431) 5S ribosomal protein [Mus musculus] |
| 798 | LIB3602-051-Q6-K1-C12 | 3717978 | BLASTX | 366 | 8.00E−35 | 55 | (Y12431) 5S ribosomal protein [Mus musculus] |
| 799 | LIB3602-093-Q6-K6-F4 | 1710551 | BLASTX | 187 | 3.00E−14 | 79 | 60S RIBOSOMAL PROTEIN L39 |
| | | | | | | | >gi\|7440765\|pir\|\|T03943 ribosomal protein L39 - |
| | | | | | | | maize >gi\|1177369\|emb\|CAA64728.1\| (X95458) |
| | | | | | | | ribosomal protein L39 [Zea mays] |
| 800 | LIB3602-084-Q6-K1-F4 | 1710551 | BLASTX | 187 | 4.00E−14 | 79 | 60S RIBOSOMAL PROTEIN L39 |
| | | | | | | | >gi\|7440765\|pir\|\|T03943 ribosomal protein L39 - |
| | | | | | | | maize >gi\|1177369\|emb\|CAA64728.1\| (X95458) |
| | | | | | | | ribosomal protein L39 [Zea mays] |
| 801 | LIB3602-062-Q6-K1-D1 | 1710551 | BLASTX | 167 | 8.00E−12 | 64 | 60S RIBOSOMAL PROTEIN L39 |
| | | | | | | | >gi\|7440765\|pir\|\|T03943 ribosomal protein L39 - |
| | | | | | | | maize >gi\|1177369\|emb\|CAA64728.1\| (X95458) |
| | | | | | | | ribosomal protein L39 [Zea mays] |
| 802 | LIB3602-012-Q6-K1-H11 | 1710551 | BLASTX | 167 | 8.00E−12 | 64 | 60S RIBOSOMAL PROTEIN L39 |
| | | | | | | | >gi\|7440765\|pir\|\|T03943 ribosomal protein L39 - |
| | | | | | | | maize >gi\|1177369\|emb\|CAA64728.1\| (X95458) |
| | | | | | | | ribosomal protein L39 [Zea mays] |
| 803 | LIB3602-040-Q6-K1-B8 | 1710551 | BLASTX | 167 | 8.00E−12 | 64 | 60S RIBOSOMAL PROTEIN L39 |
| | | | | | | | >gi\|7440765\|pir\|\|T03943 ribosomal protein L39 - |
| | | | | | | | maize >gi\|1177369\|emb\|CAA64728.1\| (X95458) |
| | | | | | | | ribosomal protein L39 [Zea mays] |
| 804 | LIB3602-014-Q6-K1-E2 | 1710551 | BLASTX | 167 | 8.00E−12 | 64 | 60S RIBOSOMAL PROTEIN L39 |
| | | | | | | | >gi\|7440765\|pir\|\|T03943 ribosomal protein L39 - |
| | | | | | | | maize >gi\|1177369\|emb\|CAA64728.1\| (X95458) |
| | | | | | | | ribosomal protein L39 [Zea mays] |
| 805 | LIB3602-022-Q6-K1-E5 | 1710551 | BLASTX | 167 | 8.00E−12 | 64 | 60S RIBOSOMAL PROTEIN L39 |
| | | | | | | | >gi\|7440765\|pir\|\|T03943 ribosomal protein L39 - |
| | | | | | | | maize >gi\|1177369\|emb\|CAA64728.1\| (X95458) |
| | | | | | | | ribosomal protein L39 [Zea mays] |
| 806 | LIB3602-032-Q6-K1-G2 | 1710551 | BLASTX | 167 | 8.00E−12 | 64 | 60S RIBOSOMAL PROTEIN L39 |
| | | | | | | | >gi\|7440765\|pir\|\|T03943 ribosomal protein L39 - |
| | | | | | | | maize >gi\|1177369\|emb\|CAA64728.1\| (X95458) |
| | | | | | | | ribosomal protein L39 [Zea mays] |
| 807 | LIB3602-102-Q1-K1-D12 | 1710551 | BLASTX | 167 | 9.00E−12 | 64 | 60S RIBOSOMAL PROTEIN L39 |
| | | | | | | | >gi\|7440765\|pir\|\|T03943 ribosomal protein L39 - |
| | | | | | | | maize >gi\|1177369\|emb\|CAA64728.1\| (X95458) |
| | | | | | | | ribosomal protein L39 [Zea mays] |
| 808 | LIB3602-045-Q6-K1-D8 | 1710551 | BLASTX | 167 | 9.00E−12 | 64 | 60S RIBOSOMAL PROTEIN L39 |
| | | | | | | | >gi\|7440765\|pir\|\|T03943 ribosomal protein L39 - |
| | | | | | | | maize >gi\|1177369\|emb\|CAA64728.1\| (X95458) |
| | | | | | | | ribosomal protein L39 [Zea mays] |
| 809 | LIB3602-055-Q6-K1-E3 | 1710551 | BLASTX | 167 | 9.00E−12 | 64 | 60S RIBOSOMAL PROTEIN L39 |
| | | | | | | | >gi\|7440765\|pir\|\|T03943 ribosomal protein L39 - |
| | | | | | | | maize >gi\|1177369\|emb\|CAA64728.1\| (X95458) |
| | | | | | | | ribosomal protein L39 [Zea mays] |
| 810 | LIB3602-043-Q6-K1-F7 | 7433374 | BLASTX | 1032 | 1.00E−112 | 85 | ferredoxin--NADP+ reductase (EC 1.18.1.2) |
| | | | | | | | precursor - Volvox carteri |
| | | | | | | | >gi\|732532\|gb\|AAB40978.1\| (U22328) ferredoxin- |
| | | | | | | | NADP+ reductase [Volvox carteri] |
| 811 | LIB3602-043-Q6-K1-E3 | 7433374 | BLASTX | 928 | 1.00E−100 | 85 | ferredoxin--NADP+ reductase (EC 1.18.1.2) |
| | | | | | | | precursor - Volvox carteri |
| | | | | | | | >gi\|732532\|gb\|AAB40978.1\| (U22328) ferredoxin- |
| | | | | | | | NADP+ reductase [Volvox carteri] |
| 812 | LIB3602-014-Q6-K1-F3 | 7433374 | BLASTX | 928 | 1.00E−100 | 85 | ferredoxin--NADP+ reductase (EC 1.18.1.2) |
| | | | | | | | precursor - Volvox carteri |
| | | | | | | | >gi\|732532\|gb\|AAB40978.1\| (U22328) ferredoxin- |
| | | | | | | | NADP+ reductase [Volvox carteri] |
| 813 | LIB3602-026-Q6-K1-H1 | 7433374 | BLASTX | 526 | 3.00E−82 | 89 | ferredoxin--NADP+ reductase (EC 1.18.1.2) |
| | | | | | | | precursor - Volvox carteri |
| | | | | | | | >gi\|732532\|gb\|AAB40978.1\| (U22328) ferredoxin- |
| | | | | | | | NADP+ reductase [Volvox carteri] |
| 814 | LIB3602-048-Q6-K1QA-D4 | 7433374 | BLASTX | 759 | 8.00E−81 | 86 | ferredoxin- NADP+ reductase (EC 1.18.1.2) |
| | | | | | | | precursor - Volvox carteri |
| | | | | | | | >gi\|732532\|gb\|AAB40978.1\| (U22328) ferredoxin- |
| | | | | | | | NADP+ reductase [Volvox carteri] |
| 815 | LIB3602-057-Q6-K1-A11 | 7433374 | BLASTX | 709 | 9.00E−75 | 84 | ferredoxin--NADP+ reductase (EC 1.18.1.2) |
| | | | | | | | precursor - Volvox carteri |
| | | | | | | | >gi\|732532\|gb\|AAB40978.1\| (U22328) ferredoxin- |
| | | | | | | | NADP+ reductase [Volvox carteri] |
| 816 | LIB3602-108-Q1-K1-F6 | 7433374 | BLASTX | 571 | 9.00E−59 | 85 | ferredoxin--NADP+ reductase (EC 1.18.1.2) |
| | | | | | | | precursor - Volvox carteri |
| | | | | | | | >gi\|732532\|gb\|AAB40978.1\| (U22328) ferredoxin- |
| | | | | | | | NADP+ reductase [Volvox carteri] |
| 817 | LIB3602-073-Q1-K1-C12 | 7433374 | BLASTX | 565 | 5.00E−58 | 78 | ferredoxin--NADP+ reductase (EC 1.18.1.2) |
| | | | | | | | precursor - Volvox carteri |
| | | | | | | | >gi\|732532\|gb\|AAB40978.1\| (U22328) ferredoxin- |
| | | | | | | | NADP+ reductase [Volvox carteri] |
| 818 | LIB3602-018-Q6-K1-A4 | 7433374 | BLASTX | 420 | 5.00E−41 | 67 | ferredoxin--NADP+ reductase (EC 1.18.1.2) |
| | | | | | | | precursor - Volvox carteri |
| | | | | | | | >gi\|732532\|gb\|AAB40978.1\| (U22328) ferredoxin- |
| | | | | | | | NADP+ reductase [Volvox carteri] |
| 819 | LIB3602-019-Q6-K1-D5 | 7433374 | BLASTX | 294 | 2.00E−26 | 84 | ferredoxin--NADP+ reductase (EC 1.18.1.2) |
| | | | | | | | precursor - Volvox carteri |
| | | | | | | | >gi\|732532\|gb\|AAB40978.1\| (U22328) ferredoxin- |
| | | | | | | | NADP+ reductase [Volvox carteri] |
| 820 | LIB3602-109-Q1-K1-A3 | 7433374 | BLASTX | 166 | 1.00E−11 | 60 | ferredoxin--NADP+ reductase (EC 1.18.1.2) |
| | | | | | | | precursor - Volvox carteri |
| | | | | | | | >gi\|732532\|gb\|AAB40978.1\| (U22328) ferredoxin- |
| | | | | | | | NADP+ reductase [Volvox carteri] |
| 821 | LIB3602-062-Q6-K1-E10 | 6831665 | BLASTX | 705 | 2.00E−74 | 83 | 40S RIBOSOMAL PROTEIN S5 |
| | | | | | | | >gi\|3043428\|emb\|CAA06491.1\| (AJ005346) 40S |
| | | | | | | | ribosomal protein S5 [Cicer arietinum] |
| 822 | LIB3602-002-P1-K6-E10 | 6831665 | BLASTX | 651 | 4.00E−68 | 88 | 40S RIBOSOMAL PROTEIN S5 |
| | | | | | | | >gi\|3043428\|emb\|CAA06491.1\| (AJ005346) 40S |
| | | | | | | | ribosomal protein S5 [Cicer arietinum] |
| 823 | LIB3602-061-Q6-K1-C9 | 6831665 | BLASTX | 642 | 4.00E−67 | 87 | 40S RIBOSOMAL PROTEIN S5 |
| | | | | | | | >gi\|3043428\|emb\|CAA06491.1\| (AJ005346) 40S |
| | | | | | | | ribosomal protein S5 [Cicer arietinum] |
| 824 | LIB3602-120-Q1-K1-B8 | 6831665 | BLASTX | 512 | 4.00E−65 | 84 | 40S RIBOSOMAL PROTEIN S5 |
| | | | | | | | >gi\|3043428\|emb\|CAA06491.1\| (AJ005346) 40S |
| | | | | | | | ribosomal protein S5 [Cicer arietinum] |
| 825 | LIB3602-089-Q6-K6-G12 | 6831665 | BLASTX | 603 | 2.00E−62 | 87 | 40S RIBOSOMAL PROTEIN S5 |
| | | | | | | | >gi\|3043428\|emb\|CAA06491.1\| (AJ005346) 40S |
| | | | | | | | ribosomal protein S5 [Cicer arietinum] |
| 826 | LIB3602-120-Q1-K1-C11 | 6831665 | BLASTX | 422 | 9.00E−60 | 91 | 40S RIBOSOMAL PROTEIN S5 |
| | | | | | | | >gi\|3043428\|emb\|CAA06491.1\| (AJ005346) 40S |
| | | | | | | | ribosomal protein S5 [Cicer arietinum] |
| 827 | LIB3602-073-Q1-K1-F1 | 6831665 | BLASTX | 500 | 2.00E−50 | 86 | 40S RIBOSOMAL PROTEIN S5 |
| | | | | | | | >gi\|3043428\|emb\|CAA06491.1\| (AJ005346) 40S |
| | | | | | | | ribosomal protein S5 [Cicer arietinum] |
| 828 | LIB3602-093-Q6-K6-E4 | 6831665 | BLASTX | 453 | 1.00E−46 | 80 | 40S RIBOSOMAL PROTEIN S5 |
| | | | | | | | >gi\|3043428\|emb\|CAA06491.1\| (AJ005346) 40S |
| | | | | | | | ribosomal protein S5 [Cicer arietinum] |
| 829 | LIB3602-102-Q1-K1-E3 | 6831665 | BLASTX | 310 | 7.00E−46 | 92 | 40S RIBOSOMAL PROTEIN S5 |
| | | | | | | | >gi\|3043428\|emb\|CAA06491.1\| (AJ005346) 40S |
| | | | | | | | ribosomal protein S5 [Cicer arietinum] |
| 830 | LIB3602-042-Q6-K1-D12 | 6831665 | BLASTX | 458 | 1.00E−45 | 89 | 40S RIBOSOMAL PROTEIN S5 |
| | | | | | | | >gi\|3043428\|emb\|CAA06491.1\| (AJ005346) 40S |
| | | | | | | | ribosomal protein S5 [Cicer arietinum] |
| 831 | LIB3602-003-Q1-K1-D1 | 6831665 | BLASTX | 387 | 2.00E−37 | 61 | 40S RIBOSOMAL PROTEIN S5 |
| | | | | | | | >gi\|3043428\|emb\|CAA06491.1\| (AJ005346) 40S |
| | | | | | | | ribosomal protein S5 [Cicer arietinum] |
| 832 | LIB3602-001-P1-K6-E10 | 4582468 | BLASTX | 827 | 9.00E−89 | 88 | (AC007071) 40S ribosomal protein; contains C- |
| | | | | | | | terminal domain [Arabidopsis thaliana] |
| 833 | LIB3602-018-Q6-K1-E3 | 4582468 | BLASTX | 733 | 9.00E−78 | 84 | (AC007071) 40S ribosomal protein; contains C- |
| | | | | | | | terminal domain [Arabidopsis thaliana] |
| 834 | LIB3602-044-Q6-K1-D7 | 4582468 | BLASTX | 725 | 7.00E−77 | 89 | (AC007071) 40S ribosomal protein; contains C- |
| | | | | | | | terminal domain [Arabidopsis thaliana] |
| 835 | LIB3602-088-Q6-K6-G12 | 4582468 | BLASTX | 693 | 5.00E−73 | 84 | (AC007071) 40S ribosomal protein; contains C- |
| | | | | | | | terminal domain [Arabidopsis thaliana] |
| 836 | LIB3602-118-Q1-K1-G12 | 4582468 | BLASTX | 551 | 2.00E−71 | 85 | (AC007071) 40S ribosomal protein; contains C- |
| | | | | | | | terminal domain [Arabidopsis thaliana] |
| 837 | LIB3602-007-Q1-K1-F1 | 4582468 | BLASTX | 584 | 3.00E−62 | 81 | (AC007071) 40S ribosomal protein; contains C- |
| | | | | | | | terminal domain [Arabidopsis thaliana] |
| 838 | LIB3602-038-Q6-K1-D4 | 4582468 | BLASTX | 484 | 1.00E−48 | 85 | (AC007071) 40S ribosomal protein; contains C- |
| | | | | | | | terminal domain [Arabidopsis thaliana] |
| 839 | LIB3602-030-Q6-K1-G10 | 4582468 | BLASTX | 349 | 6.00E−33 | 80 | (AC007071) 40S ribosomal protein; contains C- |
| | | | | | | | terminal domain [Arabidopsis thaliana] |
| 840 | LIB3602-019-Q6-K1-H6 | 4582468 | BLASTX | 341 | 5.00E−32 | 79 | (AC007071) 40S ribosomal protein; contains C- |
| | | | | | | | terminal domain [Arabidopsis thaliana] |
| 841 | LIB3602-048-Q6-K1QA-F6 | 4582468 | BLASTX | 295 | 4.00E−27 | 82 | (AC007071) 40S ribosomal protein; contains C- |
| | | | | | | | terminal domain [Arabidopsis thaliana] |
| 842 | LIB3602-015-Q6-K1-B4 | 4582468 | BLASTX | 210 | 1.00E−16 | 76 | (AC007071) 40S ribosomal protein; contains C- |
| | | | | | | | terminal domain [Arabidopsis thaliana] |
| 843 | LIB3602-015-Q6-K1-A2 | 5902596 | BLASTX | 443 | 1.00E−43 | 58 | (AF110786) light harvesting complex a protein |
| | | | | | | | [Volvox carteri f. nagariensis] |
| 844 | LIB3602-046-Q6-K1-A9 | 5902596 | BLASTX | 423 | 2.00E−41 | 57 | (AF110786) light harvesting complex a protein |
| | | | | | | | [Volvox carteri f. nagariensis] |
| 845 | LIB3602-027-Q6-K1-D2 | 5902596 | BLASTX | 344 | 4.00E−32 | 46 | (AF110786) light harvesting complex a protein |
| | | | | | | | [Volvox carteri f. nagariensis] |
| 846 | LIB3602-059-Q6-K1-A12 | 5902596 | BLASTX | 338 | 2.00E−31 | 56 | (AF110786) light harvesting complex a protein |
| | | | | | | | [Volvox carteri f. nagariensis] |
| 847 | LIB3602-007-Q1-K1-E5 | 5902596 | BLASTX | 336 | 2.00E−31 | 56 | (AF110786) light harvesting complex a protein |
| | | | | | | | [Volvox carteri f. nagariensis] |
| 848 | LIB3602-005-Q1-K1-D6 | 5902596 | BLASTX | 260 | 2.00E−22 | 42 | (AF110786) light harvesting complex a protein |
| | | | | | | | [Volvox carteri f. nagariensis] |
| 849 | LIB3602-057-Q6-K1-H11 | 5902596 | BLASTX | 211 | 1.00E−17 | 58 | (AF110786) light harvesting complex a protein |
| | | | | | | | [Volvox carteri f. nagariensis] |
| 850 | LIB3602-076-Q6-K6-E11 | 5902596 | BLASTX | 207 | 1.00E−16 | 67 | (AF110786) light harvesting complex a protein |
| | | | | | | | [Volvox carteri f. nagariensis] |
| 851 | LIB3602-095-Q6-K6-B3 | 5902596 | BLASTX | 206 | 4.00E−16 | 57 | (AF110786) light harvesting complex a protein |
| | | | | | | | [Volvox carteri f. nagariensis] |
| 852 | LIB3602-016-Q6-K1-E3 | 5902596 | BLASTX | 205 | 5.00E−16 | 67 | (AF110786) light harvesting complex a protein |
| | | | | | | | [Volvox carteri f. nagariensis] |
| 853 | LIB3602-018-Q6-K1-F11 | 5902596 | BLASTX | 194 | 1.00E−14 | 67 | (AF110786) light harvesting complex a protein |
| | | | | | | | [Volvox carteri f. nagariensis] |
| 854 | LIB3602-072-Q1-K1-F5 | 6094476 | BLASTX | 559 | 3.00E−69 | 69 | THIAZOLE BIOSYNTHETIC ENZYME |
| | | | | | | | PRECURSOR >gi\|7446894\|pir\|\|T10474 thiamin |
| | | | | | | | biosynthesis protein thi1 - sweet orange |
| | | | | | | | >gi\|2582665\|emb\|CAB05370.1\| (Z82983) thi |
| | | | | | | | [Citrus sinensis] |
| 855 | LIB3602-062-Q6-K1-H3 | 6094476 | BLASTX | 650 | 5.00E−68 | 68 | THIAZOLE BIOSYNTHETIC ENZYME |
| | | | | | | | PRECURSOR >gi\|7446894\|pir\|\|T10474 thiamin |
| | | | | | | | biosynthesis protein thi1 - sweet orange |
| | | | | | | | >gi\|2582665\|emb\|CAB05370.1\| (Z82983) thi |
| | | | | | | | [Citrus sinensis] |
| 856 | LIB3602-059-Q6-K1-C7 | 6094476 | BLASTX | 622 | 9.00E−65 | 67 | THIAZOLE BIOSYNTHETIC ENZYME |
| | | | | | | | PRECURSOR >gi\|7446894\|pir\|\|T10474 thiamin |
| | | | | | | | biosynthesis protein thi1 - sweet orange |
| | | | | | | | >gi\|2582665\|emb\|CAB05370.1\| (Z82983) thi |
| | | | | | | | [Citrus sinensis] |
| 857 | LIB3602-065-Q1-K6-B5 | 6094476 | BLASTX | 598 | 6.00E−62 | 66 | THIAZOLE BIOSYNTHETIC ENZYME |
| | | | | | | | PRECURSOR >gi\|7446894\|pir\|\|T10474 thiamin |
| | | | | | | | biosynthesis protein thi1 - sweet orange |
| | | | | | | | >gi\|2582665\|emb\|CAB05370.1 (Z82983) thi |
| | | | | | | | [Citrus sinensis] |
| 858 | LIB3602-103-Q1-K1-G3 | 6094476 | BLASTX | 585 | 2.00E−60 | 66 | THIAZOLE BIOSYNTHETIC ENZYME |
| | | | | | | | PRECURSOR >gi\|7446894\|pir\|\|T10474 thiamin |
| | | | | | | | biosynthesis protein thi1 - sweet orange |
| | | | | | | | >gi\|2582665\|emb\|CAB05370.1\| (Z82983) thi |
| | | | | | | | [Citrus sinensis] |
| 859 | LIB3602-111-Q1-K1-D5 | 6094476 | BLASTX | 482 | 3.00E−59 | 72 | THIAZOLE BIOSYNTHETIC ENZYME |
| | | | | | | | PRECURSOR >gi\|7446894\|pir\|\|T10474 thiamin |
| | | | | | | | biosynthesis protein thi1 - sweet orange |
| | | | | | | | >gi\|2582665\|emb\|CAB05370.1\| (Z82983) thi |
| | | | | | | | [Citrus sinensis] |
| 860 | LIB3602-044-Q6-K1-G8 | 6094476 | BLASTX | 553 | 1.00E−56 | 70 | THIAZOLE BIOSYNTHETIC ENZYME |
| | | | | | | | PRECURSOR >gi\|7446894\|pir\|\|T10474 thiamin |
| | | | | | | | biosynthesis protein thi1 - sweet orange |
| | | | | | | | >gi\|2582665\|emb\|CAB05370.1\| (Z82983) thi |
| | | | | | | | [Citrus sinensis] |
| 861 | LIB3602-030-Q6-K1-G5 | 6094476 | BLASTX | 533 | 2.00E−54 | 65 | THIAZOLE BIOSYNTHETIC ENZYME |
| | | | | | | | PRECURSOR >gi\|7446894\|pir\|\|T10474 thiamin |
| | | | | | | | biosynthesis protein thi1 - sweet orange |
| | | | | | | | >gi\|2582665\|emb\|CAB05370.1\| (Z82983) thi |
| | | | | | | | [Citrus sinensis] |
| 862 | LIB3602-054-Q6-K1-B11 | 6094476 | BLASTX | 448 | 2.00E−44 | 73 | THIAZOLE BIOSYNTHETIC ENZYME |
| | | | | | | | PRECURSOR >gi\|7446894\|pir\|\|T10474 thiamin |
| | | | | | | | biosynthesis protein thi1 - sweet orange |
| | | | | | | | >gi\|2582665\|emb\|CAB05370.1\| (Z82983) thi |
| | | | | | | | [Citrus sinensis] |
| 863 | LIB3602-107-Q1-K1-G8 | 6094476 | BLASTX | 160 | 2.00E−10 | 73 | THIAZOLE BIOSYNTHETIC ENZYME |
| | | | | | | | PRECURSOR >gi\|7446894\|pir\|\|T10474 thiamin |
| | | | | | | | biosynthesis protein thi1 - sweet orange |
| | | | | | | | >gi\|2582665\|emb\|CAB05370.1\| (Z82983) thi |
| | | | | | | | [Citrus sinensis] |
| 864 | LIB3602-008-Q6-K1-H7 | 1173024 | BLASTX | 364 | 1.00E−34 | 66 | 60S RIBOSOMAL PROTEIN L31 |
| | | | | | | | >gi\|99421\|pir\|\|S24989 ribosomal protein L31.e, |
| | | | | | | | cytosolic - Chlamydomonas reinhardtii |
| | | | | | | | >gi\|18209\|emb\|CAA47044.1\| (X66413) ribosomal |
| | | | | | | | protein L31 [Chlamydomonas reinhardtii] |
| 865 | LIB3602-071-Q1-K1-F2 | 1173024 | BLASTX | 360 | 4.00E−34 | 65 | 60S RIBOSOMAL PROTEIN L31 |
| | | | | | | | >gi\|99421\|pir\|\|S24989 ribosomal protein L31.e, |
| | | | | | | | cytosolic - Chlamydomonas reinhardtii |
| | | | | | | | >gi\|18209\|emb\|CAA47044.1\| (X66413) ribosomal |
| | | | | | | | protein L31 [Chlamydomonas reinhardtii] |
| 866 | LIB3602-095-Q6-K6-B11 | 1173024 | BLASTX | 360 | 5.00E−34 | 65 | 60S RIBOSOMAL PROTEIN L31 |
| | | | | | | | >gi\|99421\|pir\|\|S24989 ribosomal protein L31.e, |
| | | | | | | | cytosolic - Chlamydomonas reinhardtii |
| | | | | | | | >gi\|18209\|emb\|CAA47044.1\| (X66413) ribosomal |
| | | | | | | | protein L31 [Chlamydomonas reinhardtii] |
| 867 | LIB3602-087-Q6-K1-C1 | 1173024 | BLASTX | 358 | 8.00E−34 | 67 | 60S RIBOSOMAL PROTEIN L31 |
| | | | | | | | >gi\|99421\|pir\|\|S24989 ribosomal protein L31.e, |
| | | | | | | | cytosolic - Chlamydomonas reinhardtii |
| | | | | | | | >gi\|18209\|emb\|CAA47044.1\| (X66413) ribosomal |
| | | | | | | | protein L31 [Chlamydomonas reinhardtii] |
| 868 | LIB3602-067-Q1-K1-C11 | 1173024 | BLASTX | 358 | 8.00E−34 | 67 | 60S RIBOSOMAL PROTEIN L31 |
| | | | | | | | >gi\|99421\|pir\|\|S24989 ribosomal protein L31.e, |
| | | | | | | | cytosolic - Chlamydomonas reinhardtii |
| | | | | | | | >gi\|18209\|emb\|CAA47044.1\| (X66413) ribosomal |
| | | | | | | | protein L31 [Chlamydomonas reinhardtii] |
| 869 | LIB3602-105-Q1-K1-D2 | 1173024 | BLASTX | 342 | 7.00E−32 | 66 | 60S RIBOSOMAL PROTEIN L31 |
| | | | | | | | >gi\|99421\|pir\|\|S24989 ribosomal protein L31.e, |
| | | | | | | | cytosolic - Chlamydomonas reinhardtii |
| | | | | | | | >gi\|18209\|emb\|CAA47044.1\| (X66413) ribosomal |
| | | | | | | | protein L31 [Chlamydomonas reinhardtii] |
| 870 | LIB3602-105-Q1-K1-C12 | 1173024 | BLASTX | 340 | 1.00E−31 | 62 | 60S RIBOSOMAL PROTEIN L31 |
| | | | | | | | >gi\|99421\|pir\|\|S24989 ribosomal protein L31.e, |
| | | | | | | | cytosolic - Chlamydomonas reinhardtii |
| | | | | | | | >gi\|18209\|emb\|CAA47044.1\| (X66413) ribosomal |
| | | | | | | | protein L31 [Chlamydomonas reinhardtii] |
| 871 | LIB3602-020-Q6-K1-D9 | 1173024 | BLASTX | 269 | 1.00E−23 | 67 | 60S RIBOSOMAL PROTEIN L31 |
| | | | | | | | >gi\|99421\|pir\|\|S24989 ribosomal protein L31.e, |
| | | | | | | | cytosolic - Chlamydomonas reinhardtii |
| | | | | | | | >gi\|18209\|emb\|CAA47044.1\| (X66413) ribosomal |
| | | | | | | | protein L31 [Chlamydomonas reinhardtii] |
| 872 | LIB3602-057-Q6-K1-E11 | 1173024 | BLASTX | 221 | 7.00E−18 | 51 | 60S RIBOSOMAL PROTEIN L31 |
| | | | | | | | >gi\|99421\|pir\|\|S24989 ribosomal protein L31.e, |
| | | | | | | | cytosolic - Chlamydomonas reinhardtii |
| | | | | | | | >gi\|18209\|emb\|CAA47044.1\| (X66413) ribosomal |
| | | | | | | | protein L31 [Chlamydomonas reinhardtii] |
| 873 | LIB3602-022-Q6-K1-E6 | 1173024 | BLASTX | 177 | 9.00E−13 | 48 | 60S RIBOSOMAL PROTEIN L31 |
| | | | | | | | >gi\|99421\|pir\|\|S24989 ribosomal protein L31.e, |
| | | | | | | | cytosolic - Chlamydomonas reinhardtii |
| | | | | | | | >gi\|18209\|emb\|CAA47044.1\| (X66413) ribosomal |
| | | | | | | | protein L31 [Chlamydomonas reinhardtii] |
| 874 | LIB3602-047-Q6-K1-A9 | 7269837 | BLASTX | 261 | 2.00E−22 | 84 | (AL161574) RIBOSOMAL PROTEIN S30 |
| | | | | | | | homolog [Arabidopsis thaliana] |
| 875 | LIB3602-043-Q6-K1-B4 | 7269837 | BLASTX | 261 | 2.00E−22 | 84 | (AL161574) RIBOSOMAL PROTEIN S30 |
| | | | | | | | homolog [Arabidopsis thaliana] |
| 876 | LIB3602-042-Q6-K1-F1 | 7269837 | BLASTX | 261 | 2.00E−22 | 84 | (AL161574) RIBOSOMAL PROTEIN S30 |
| | | | | | | | homolog [Arabidopsis thaliana] |
| 877 | LIB3602-032-Q6-K1-H4 | 7269837 | BLASTX | 261 | 2.00E−22 | 84 | (AL161574) RIBOSOMAL PROTEIN S30 |
| | | | | | | | homolog [Arabidopsis thaliana] |
| 878 | LIB3602-058-Q6-K1-B12 | 7269837 | BLASTX | 261 | 2.00E−22 | 84 | (AL161574) RIBOSOMAL PROTEIN S30 |
| | | | | | | | homolog [Arabidopsis thaliana] |
| 879 | LIB3602-019-Q6-K1-C8 | 7269837 | BLASTX | 258 | 3.00E−22 | 84 | (AL161574) RIBOSOMAL PROTEIN S30 |
| | | | | | | | homolog [Arabidopsis thaliana] |
| 880 | LIB3602-111-Q1-K1-G2 | 7269837 | BLASTX | 258 | 3.00E−22 | 83 | (AL161574) RIBOSOMAL PROTEIN S30 |
| | | | | | | | homolog [Arabidopsis thaliana] |
| 881 | LIB3602-109-Q1-K1-B11 | 7269837 | BLASTX | 252 | 2.00E−21 | 83 | (AL161574) RIBOSOMAL PROTEIN S30 |
| | | | | | | | homolog [Arabidopsis thaliana] |
| 882 | LIB3602-032-Q6-K1-A4 | 7269837 | BLASTX | 170 | 6.00E−12 | 78 | (AL161574) RIBOSOMAL PROTEIN S30 |
| | | | | | | | homolog [Arabidopsis thaliana] |
| 883 | LIB3602-046-Q6-K1-F8 | 7269837 | BLASTX | 156 | 3.00E−10 | 65 | (AL161574) RIBOSOMAL PROTEIN S30 |
| | | | | | | | homolog [Arabidopsis thaliana] |
| 884 | LIB3602-104-Q1-K1-G6 | 133808 | BLASTX | 494 | 1.00E−49 | 77 | 40S RIBOSOMAL PROTEIN S16 |
| | | | | | | | >gi\|70921\|pir\|\|R3YL16 ribosomal protein S16, |
| | | | | | | | cytosolic - large-leaved lupine |
| | | | | | | | >gi\|19512\|emb\|CAA36068.1\| (X51766) rps16 gene |
| | | | | | | | product (AA 1-145) [Lupinus polyphyllus] |
| 885 | LIB3602-113-Q1-K1-C8 | 133808 | BLASTX | 445 | 5.00E−44 | 81 | 40S RIBOSOMAL PROTEIN S16 |
| | | | | | | | >gi\|70921\|pir\|\|R3YL16 ribosomal protein S16, |
| | | | | | | | cytosolic - large-leaved lupine |
| | | | | | | | >gi\|19512\|emb\|CAA36068.1\| (X51766) rps16 gene |
| | | | | | | | product (AA 1-145) [Lupinus polyphyllus] |
| 886 | LIB3602-065-Q1-K6-A4 | 133808 | BLASTX | 434 | 9.00E−43 | 83 | 40S RIBOSOMAL PROTEIN S16 |
| | | | | | | | >gi\|70921\|pir\|\|R3YL16 ribosomal protein S16, |
| | | | | | | | cytosolic - large-leaved lupine |
| | | | | | | | >gi\|19512\|emb\|CAA36068.1\| (X51766) rps16 gene |
| | | | | | | | product (AA 1-145) [Lupinus polyphyllus] |
| 887 | LIB3602-120-Q1-K1-C8 | 133808 | BLASTX | 270 | 6.00E−41 | 87 | 40S RIBOSOMAL PROTEIN S16 |
| | | | | | | | >gi\|70921\|pir\|\|R3YL16 ribosomal protein S16, |
| | | | | | | | cytosolic - large-leaved lupine |
| | | | | | | | >gi\|19512\|emb\|CAA36068.1\| (X51766) rps16 gene |
| | | | | | | | product (AA 1-145) [Lupinus polyphyllus] |
| 888 | LIB3602-052-Q6-K1-G8 | 133808 | BLASTX | 354 | 9.00E−39 | 87 | 40S RIBOSOMAL PROTEIN S16 |
| | | | | | | | >gi\|70921\|pir\|\|R3YL16 ribosomal protein S16, |
| | | | | | | | cytosolic - large-leaved lupine |
| | | | | | | | >gi\|19512\|emb\|CAA36068.1\| (X51766) rps16 gene |
| | | | | | | | product (AA 1-145) [Lupinus polyphyllus] |
| 889 | LIB3602-027-Q6-K1-F8 | 133808 | BLASTX | 365 | 9.00E−35 | 86 | 40S RIBOSOMAL PROTEIN S16 |
| | | | | | | | >gi\|70921\|pir\|\|R3YL16 ribosomal protein S16, |
| | | | | | | | cytosolic - large-leaved lupine |
| | | | | | | | >gi\|19512\|emb\|CAA36068.1\| (X51766) rps16 gene |
| | | | | | | | product (AA 1-145) [Lupinus polyphyllus] |
| 890 | LIB3602-042-Q6-K1-A8 | 133808 | BLASTX | 354 | 2.00E−33 | 87 | 40S RIBOSOMAL PROTEIN S16 |
| | | | | | | | >gi\|70921\|pir\|\|R3YL16 ribosomal protein S16, |
| | | | | | | | cytosolic - large-leaved lupine |
| | | | | | | | >gi\|19512\|emb\|CAA36068.1\| (X51766) rps16 gene |
| | | | | | | | product (AA 1-145) [Lupinus polyphyllus] |
| 891 | LIB3602-088-Q6-K6-E2 | 133808 | BLASTX | 240 | 2.00E−31 | 80 | 40S RIBOSOMAL PROTEIN S16 |
| | | | | | | | >gi\|70921\|pir\|\|R3YL16 ribosomal protein S16, |
| | | | | | | | cytosolic - large-leaved lupine |
| | | | | | | | >gi\|19512\|emb\|CAA36068.1\| (X51766) rps16 gene |
| | | | | | | | product (AA 1-145) [Lupinus polyphyllus] |
| 892 | LIB3602-076-Q6-K6-A9 | 133808 | BLASTX | 265 | 7.00E−23 | 87 | 40S RIBOSOMAL PROTEIN S16 |
| | | | | | | | >gi\|70921\|pir\|\|R3YL16 ribosomal protein S16, |
| | | | | | | | cytosolic - large-leaved lupine |
| | | | | | | | >gi\|19512\|emb\|CAA36068.1\| (X51766) rps16 gene |
| | | | | | | | product (AA 1-145) [Lupinus polyphyllus] |
| 893 | LIB3602-095-Q6-K6-A2 | 133808 | BLASTX | 256 | 3.00E−22 | 90 | 40S RIBOSOMAL PROTEIN S16 |
| | | | | | | | >gi\|70921\|pir\|\|R3YL16 ribosomal protein S16, |
| | | | | | | | cytosolic - large-leaved lupine |
| | | | | | | | >gi\|19512\|emb\|CAA36068.1\| (X51766) rps16 gene |
| | | | | | | | product (AA 1-145) [Lupinus polyphyllus] |
| 894 | LIB3602-061-Q6-K1-A11 | 130263 | BLASTX | 407 | 1.00E−39 | 76 | PLASTOCYANIN >gi\|65845\|pir\|CUKLCF |
| | | | | | | | plastocyanin - Chlorella fusca |
| 895 | LIB3602-113-Q1-K1-E2 | 130263 | BLASTX | 407 | 2.00E−39 | 76 | PLASTOCYANIN >gi\|65845\|pir\|\|CUKLCF |
| | | | | | | | plastocyanin - Chlorella fusca |
| 896 | LIB3602-035-Q1-K1-A9 | 130263 | BLASTX | 407 | 2.00E−39 | 76 | PLASTOCYANIN >gi\|65845\|pir\|\|CUKLCF |
| | | | | | | | plastocyanin - Chlorella fusca |
| 897 | LIB3602-054-Q6-K1-D4 | 130263 | BLASTX | 401 | 6.00E−39 | 75 | PLASTOCYANIN >gi\|65845\|pir\|\|CUKLCF |
| | | | | | | | plastocyanin - Chlorella fusca |
| 898 | LIB3602-092-Q6-K6-G3 | 130263 | BLASTX | 399 | 2.00E−38 | 75 | PLASTOCYANIN >gi\|65845\|pir\|\|CUKLCF |
| | | | | | | | plastocyanin - Chlorella fusca |
| 899 | LIB3602-089-Q6-K6-A3 | 130263 | BLASTX | 397 | 2.00E−38 | 75 | PLASTOCYANIN >gi\|65845\|pir\|\|CUKLCF |
| | | | | | | | plastocyanin - Chlorella fusca |
| 900 | LIB3602-073-Q1-K1-A5 | 130263 | BLASTX | 323 | 4.00E−34 | 78 | PLASTOCYANIN >gi\|65845\|pir\|\|CUKLCF |
| | | | | | | | plastocyanin - Chlorella fusca |
| 901 | LIB3602-087-Q6-K1-G9 | 130263 | BLASTX | 232 | 5.00E−32 | 68 | PLASTOCYANIN >gi\|65845\|pir\|\|CUKLCF |
| | | | | | | | plastocyanin - Chlorella fusca |
| 902 | LIB3602-034-Q6-K1-A9 | 130263 | BLASTX | 302 | 2.00E−27 | 76 | PLASTOCYANIN >gi\|65845\|pir\|\|CUKLCF |
| | | | | | | | plastocyanin - Chlorella fusca |
| 903 | LIB3602-062-Q6-K1-C6 | 130263 | BLASTX | 232 | 3.00E−19 | 71 | PLASTOCYANIN >gi\|65845\|pir\|\|CUKLCF |
| | | | | | | | plastocyanin - Chlorella fusca |
| 904 | LIB3602-078-Q6-K6-E10 | 7441093 | BLASTX | 500 | 2.00E−50 | 72 | ribosomal protein L32, cytosolic - Arabidopsis |
| | | | | | | | thaliana >gi\|5816996\|emb\|CAB53651.1\| |
| | | | | | | | (AL110123) ribosomal protein L32-like protein |
| | | | | | | | [Arabidopsis thaliana] |
| | | | | | | | >gi\|7268562\|emb\|CAB78812.1\| (AL161547) |
| | | | | | | | ribosomal protein L32-like protein [Arabidopsis |
| | | | | | | | thaliana] |
| 905 | LIB3602-053-Q6-K1-F1 | 7441093 | BLASTX | 500 | 2.00E−50 | 72 | ribosomal protein L32, cytosolic - Arabidopsis |
| | | | | | | | thaliana >gi\|5816996\|emb\|CAB53651.1\| |
| | | | | | | | (AL110123) ribosomal protein L32-like protein |
| | | | | | | | [Arabidopsis thaliana] |
| | | | | | | | >gi\|7268562\|emb\|CAB78812.1\| (AL161547) |
| | | | | | | | ribosomal protein L32-like protein [Arabidopsis |
| | | | | | | | thaliana] |
| 906 | LIB3602-066-Q1-K6-G5 | 7441093 | BLASTX | 492 | 2.00E−49 | 72 | ribosomal protein L32, cytosolic - Arabidopsis |
| | | | | | | | thaliana >gi\|5816996\|emb\|CAB53651.1\| |
| | | | | | | | (AL110123) ribosomal protein L32-like protein |
| | | | | | | | [Arabidopsis thaliana] |
| | | | | | | | >gi\|7268562\|emb\|CAB78812.1\| (AL161547) |
| | | | | | | | ribosomal protein L32-like protein [Arabidopsis |
| | | | | | | | thaliana] |
| 907 | LIB3602-077-Q6-K6-G8 | 7441093 | BLASTX | 373 | 5.00E−45 | 73 | ribosomal protein L32, cytosolic - Arabidopsis |
| | | | | | | | thaliana >gi\|5816996\|emb\|CAB53651.1\| |
| | | | | | | | (AL110123) ribosomal protein L32-like protein |
| | | | | | | | [Arabidopsis thaliana] |
| | | | | | | | >gi\|7268562\|emb\|CAB78812.1\| (AL161547) |
| | | | | | | | ribosomal protein L32-like protein [Arabidopsis |
| | | | | | | | thaliana] |
| 908 | LIB3602-040-Q6-K1-A12 | 7441093 | BLASTX | 452 | 6.00E−45 | 77 | ribosomal protein L32, cytosolic - Arabidopsis |
| | | | | | | | thaliana >gi\|5816996\|emb\|CAB53651.1\| |
| | | | | | | | (AL110123) ribosomal protein L32-like protein |
| | | | | | | | [Arabidopsis thaliana] |
| | | | | | | | >gi\|7268562\|emb\|CAB78812.1\| (AL161547) |
| | | | | | | | ribosomal protein L32-like protein [Arabidopsis |
| | | | | | | | thaliana] |
| 909 | LIB3602-003-Q1-K1-C10 | 7441093 | BLASTX | 356 | 9.00E−34 | 73 | ribosomal protein L32, cytosolic - Arabidopsis |
| | | | | | | | thaliana >gi\|5816996\|emb\|CAB53651.1\| |
| | | | | | | | (AL110123) ribosomal protein L32-like protein |
| | | | | | | | [Arabidopsis thaliana] |
| | | | | | | | >gi\|7268562\|emb\|CAB78812.1\| (AL161547) |
| | | | | | | | ribosomal protein L32-like protein [Arabidopsis |
| | | | | | | | thaliana] |
| 910 | LIB3602-065-Q1-K6-D6 | 7441093 | BLASTX | 278 | 3.00E−33 | 70 | ribosomal protein L32, cytosolic - Arabidopsis |
| | | | | | | | thaliana >gi\|5816996\|emb\|CAB53651.1\| |
| | | | | | | | (AL110123) ribosomal protein L32-like protein |
| | | | | | | | [Arabidopsis thaliana] |
| | | | | | | | >gi\|7268562\|emb\|CAB78812.1\| (AL161547) |
| | | | | | | | ribosomal protein L32-like protein [Arabidopsis |
| | | | | | | | thaliana] |
| 911 | LIB3602-003-Q1-K1-C11 | 7441093 | BLASTX | 283 | 3.00E−25 | 67 | ribosomal protein L32, cytosolic - Arabidopsis |
| | | | | | | | thaliana >gi\|5816996\|emb\|CAB53651.1\| |
| | | | | | | | (AL110123) ribosomal protein L32-like protein |
| | | | | | | | [Arabidopsis thaliana] |
| | | | | | | | >gi\|7268562\|emb\|CAB78812.1\| (AL161547) |
| | | | | | | | ribosomal protein L32-like protein [Arabidopsis |
| | | | | | | | thaliana] |
| 912 | LIB3602-032-Q6-K1-E2 | 7441093 | BLASTX | 201 | 1.00E−15 | 75 | ribosomal protein L32, cytosolic - Arabidopsis |
| | | | | | | | thaliana >gi\|5816996\|emb\|CAB53651.1\| |
| | | | | | | | (AL110123) ribosomal protein L32-like protein |
| | | | | | | | [Arabidopsis thaliana] |
| | | | | | | | >gi\|7268562\|emb\|CAB78812.1\| (AL161547) |
| | | | | | | | ribosomal protein L32-like protein [Arabidopsis |
| | | | | | | | thaliana] |
| 913 | LIB3602-043-Q6-K1-A12 | 7441093 | BLASTX | 199 | 1.00E−15 | 76 | ribosomal protein L32, cytosolic - Arabidopsis |
| | | | | | | | thaliana >gi\|5816996\|emb\|CAB53651.1\| |
| | | | | | | | (AL110123) ribosomal protein L32-like protein |
| | | | | | | | [Arabidopsis thaliana] |
| | | | | | | | >gi\|7268562\|emb\|CAB78812.1\| (AL161547) |
| | | | | | | | ribosomal protein L32-like protein [Arabidopsis |
| | | | | | | | thaliana] |
| 914 | LIB3602-077-Q6-K6-E8 | 3914685 | BLASTX | 563 | 9.00E−58 | 69 | 60S RIBOSOMAL PROTEIN L17 |
| | | | | | | | >gi\|7440981\|pir\|\|T01410 ribosomal protein L17 - |
| | | | | | | | maize >gi\|2668748\|gb\|AAB88619.1\| (AF034948) |
| | | | | | | | ribosomal protein L17 [Zea mays] |
| 915 | LIB3602-058-Q6-K1-A8 | 3914685 | BLASTX | 556 | 5.00E−57 | 71 | 60S RIBOSOMAL PROTEIN L17 |
| | | | | | | | >gi\|7440981\|pir\|\|T01410 ribosomal protein L17 - |
| | | | | | | | maize >gi\|2668748\|gb\|AAB88619.1\| (AF034948) |
| | | | | | | | ribosomal protein L17 [Zea mays] |
| 916 | LIB3602-073-Q1-K1-G2 | 3914685 | BLASTX | 556 | 6.00E−57 | 71 | 60S RIBOSOMAL PROTEIN L17 |
| | | | | | | | >gi\|7440981\|pir\|\|T01410 ribosomal protein L17 - |
| | | | | | | | maize >gi\|2668748\|gb\|AAB88619.1\| (AF034948) |
| | | | | | | | ribosomal protein L17 [Zea mays] |
| 917 | LIB3602-028-Q6-K1-E6 | 3914685 | BLASTX | 545 | 1.00E−55 | 72 | 60S RIBOSOMAL PROTEIN L17 |
| | | | | | | | >gi\|7440981\|pir\|\|T01410 ribosomal protein L17 - |
| | | | | | | | maize >gi\|2668748\|gb\|AAB88619.1\| (AF034948) |
| | | | | | | | ribosomal protein L17 [Zea mays] |
| 918 | LIB3602-102-Q1-K1-G3 | 3914685 | BLASTX | 542 | 2.00E−55 | 72 | 60S RIBOSOMAL PROTEIN L17 |
| | | | | | | | >gi\|7440981\|pir\|\|T01410 ribosomal protein L17 - |
| | | | | | | | maize >gi\|2668748\|gb\|AAB88619.1\| (AF034948) |
| | | | | | | | ribosomal protein L17 [Zea mays] |
| 919 | LIB3602-041-Q6-K1-E2 | 3914685 | BLASTX | 542 | 2.00E−55 | 72 | 60S RIBOSOMAL PROTEIN L17 |
| | | | | | | | >gi\|7440981\|pir\|\|T01410 ribosomal protein L17 - |
| | | | | | | | maize >gi\|2668748\|gb\|AAB88619.1\| (AF034948) |
| | | | | | | | ribosomal protein L17 [Zea mays] |
| 920 | LIB3602-079-Q6-K6-F1 | 3914685 | BLASTX | 542 | 3.00E−55 | 72 | 60S RIBOSOMAL PROTEIN L17 |
| | | | | | | | >gi\|7440981\|pir\|\|T01410 ribosomal protein L17 - |
| | | | | | | | maize >gi\|2668748\|gb\|AAB88619.1\| (AF034948) |
| | | | | | | | ribosomal protein L17 [Zea mays] |
| 921 | LIB3602-088-Q6-K6-C3 | 3914685 | BLASTX | 495 | 3.00E−51 | 70 | 60S RIBOSOMAL PROTEIN L17 |
| | | | | | | | >gi\|7440981\|pir\|\|T01410 ribosomal protein L17 - |
| | | | | | | | maize >gi\|2668748\|gb\|AAB88619.1\| (AF034948) |
| | | | | | | | ribosomal protein L17 [Zea mays] |
| 922 | LIB3602-100-Q1-K1-H8 | 3914685 | BLASTX | 382 | 5.00E−50 | 74 | 60S RIBOSOMAL PROTEIN L17 |
| | | | | | | | >gi\|7440981\|pir\|\|T01410 ribosomal protein L17 - |
| | | | | | | | maize >gi\|2668748\|gb\|AAB88619.1\| (AF034948) |
| | | | | | | | ribosomal protein L17 [Zea mays] |
| 923 | LIB3602-014-Q6-K1-C5 | 3914685 | BLASTX | 450 | 1.00E−44 | 71 | 60S RIBOSOMAL PROTEIN L17 |
| | | | | | | | >gi\|7440981\|pir\|\|T01410 ribosomal protein L17 - |
| | | | | | | | maize >gi\|2668748\|gb\|AAB88619.1\| (AF034948) |
| | | | | | | | ribosomal protein L17 [Zea mays] |
| 924 | LIB3602-017-Q6-K1-B1 | 6984142 | BLASTX | 315 | 8.00E−29 | 82 | (AF227626) 40S ribosomal protein S11 [Euphorbia |
| | | | | | | | esula] |
| 925 | LIB3602-102-Q1-K1-C10 | 6984142 | BLASTX | 311 | 2.00E−28 | 83 | (AF227626) 40S ribosomal protein S11 [Euphorbia |
| | | | | | | | esula] |
| 926 | LIB3602-078-Q6-K6-H10 | 6984142 | BLASTX | 309 | 4.00E−28 | 75 | (AF227626) 40S ribosomal protein S11 [Euphorbia |
| | | | | | | | esula] |
| 927 | LIB3602-070-Q1-K1-A8 | 6984142 | BLASTX | 306 | 9.00E−28 | 83 | (AF227626) 40S ribosomal protein S11 [Euphorbia |
| | | | | | | | esula] |
| 928 | LIB3602-066-Q1-K6-D11 | 6984142 | BLASTX | 306 | 9.00E−28 | 83 | (AF227626) 40S ribosomal protein S11 [Euphorbia |
| | | | | | | | esula] |
| 929 | LIB3602-100-Q1-K1-H4 | 6984142 | BLASTX | 202 | 4.00E−23 | 81 | (AF227626) 40S ribosomal protein S11 [Euphorbia |
| | | | | | | | esula] |
| 930 | LIB3602-067-Q1-K1-B2 | 6984142 | BLASTX | 203 | 5.00E−23 | 82 | (AF227626) 40S ribosomal protein S11 [Euphorbia |
| | | | | | | | esula] |
| 931 | LIB3602-064-Q1-K6-G6 | 6984142 | BLASTX | 251 | 2.00E−21 | 77 | (AF227626) 40S ribosomal protein S11 [Euphorbia |
| | | | | | | | esula] |
| 932 | LIB3602-079-Q6-K6-D11 | 6984142 | BLASTX | 211 | 3.00E−17 | 77 | (AF227626) 40S ribosomal protein S11 [Euphorbia |
| | | | | | | | esula] |
| 933 | LIB3602-053-Q6-K1-D9 | 121026 | BLASTX | 942 | 1.00E−102 | 88 | GUANINE NUCLEOTIDE-BINDING PROTEIN |
| | | | | | | | BETA SUBUNIT-LIKE PROTEIN |
| | | | | | | | >gi\|99395\|pir\|\|S11904 GTP-binding regulatory |
| | | | | | | | protein beta chain homolog - Chlamydomonas |
| | | | | | | | reinhardtii >gi\|18145\|emb\|CAA37638.1\| (X53574) |
| | | | | | | | putative protein has homology to G protein beta |
| | | | | | | | subunit [Ch |
| 934 | LIB3602-105-Q1-K1-B4 | 121026 | BLASTX | 845 | 9.00E−95 | 91 | GUANINE NUCLEOTIDE-BINDING PROTEIN |
| | | | | | | | BETA SUBUNIT-LIKE PROTEIN |
| | | | | | | | >gi\|99395\|pir\|\|S11904 GTP-binding regulatory |
| | | | | | | | protein beta chain homolog - Chlamydomonas |
| | | | | | | | reinhardtii >gi\|18145\|emb\|CAA37638.1\| (X53574) |
| | | | | | | | putative protein has homology to G protein beta |
| | | | | | | | subunit [Ch |
| 935 | LIB3602-072-Q1-K1-E1 | 121026 | BLASTX | 792 | 1.00E−84 | 87 | GUANINE NUCLEOTIDE-BINDING PROTEIN |
| | | | | | | | BETA SUBUNIT-LIKE PROTEIN |
| | | | | | | | >gi\|99395\|pir\|\|S11904 GTP-binding regulatory |
| | | | | | | | protein beta chain homolog - Chlamydomonas |
| | | | | | | | reinhardtii >gi\|18145\|emb\|CAA37638.1\| (X53574) |
| | | | | | | | putative protein has homology to G protein beta |
| | | | | | | | subunit [Ch |
| 936 | LIB3602-053-Q6-K1-F7 | 121026 | BLASTX | 700 | 6.00E−74 | 86 | GUANINE NUCLEOTIDE-BINDING PROTEIN |
| | | | | | | | BETA SUBUNIT-LIKE PROTEIN |
| | | | | | | | >gi\|99395\|pir\|\|S11904 GTP-binding regulatory |
| | | | | | | | protein beta chain homolog - Chlamydomonas |
| | | | | | | | reinhardtii >gi\|18145\|emb\|CAA37638.1\| (X53574) |
| | | | | | | | putative protein has homology to G protein beta |
| | | | | | | | subunit [Ch |
| 937 | LIB3602-043-Q6-K1-D1 | 121026 | BLASTX | 570 | 1.00E−58 | 88 | GUANINE NUCLEOTIDE-BINDING PROTEIN |
| | | | | | | | BETA SUBUNIT-LIKE PROTEIN |
| | | | | | | | >gi\|99395\|pir\|\|S11904 GTP-binding regulatory |
| | | | | | | | protein beta chain homolog - Chlamydomonas |
| | | | | | | | reinhardtii >gi\|18145\|emb\|CAA37638.1\| (X53574) |
| | | | | | | | putative protein has homology to G protein beta |
| | | | | | | | subunit [Ch |
| 938 | LIB3602-059-Q6-K1-G12 | 121026 | BLASTX | 566 | 4.00E−58 | 88 | GUANINE NUCLEOTIDE-BINDING PROTEIN |
| | | | | | | | BETA SUBUNIT-LIKE PROTEIN |
| | | | | | | | >gi\|99395\|pir\|\|S11904 GTP-binding regulatory |
| | | | | | | | protein beta chain homolog - Chlamydomonas |
| | | | | | | | reinhardtii >gi\|18145\|emb\|CAA37638.1\| (X53574) |
| | | | | | | | putative protein has homology to G protein beta |
| | | | | | | | subunit [Ch |
| 939 | LIB3602-101-Q1-K1-E1 | 121026 | BLASTX | 244 | 2.00E−20 | 83 | GUANINE NUCLEOTIDE-BINDING PROTEIN |
| | | | | | | | BETA SUBUNIT-LIKE PROTEIN |
| | | | | | | | >gi\|99395\|pir\|\|S11904 GTP-binding regulatory |
| | | | | | | | protein beta chain homolog - Chlamydomonas |
| | | | | | | | reinhardtii >gi\|18145\|emb\|CAA37638.1\| (X53574) |
| | | | | | | | putative protein has homology to G protein beta |
| | | | | | | | subunit [Ch |
| 940 | LIB3602-071-Q1-K1-A1 | 121026 | BLASTX | 214 | 6.00E−17 | 82 | GUANINE NUCLEOTIDE-BINDING PROTEIN |
| | | | | | | | BETA SUBUNIT-LIKE PROTEIN |
| | | | | | | | >gi\|99395\|pir\|\|S11904 GTP-binding regulatory |
| | | | | | | | protein beta chain homolog - Chlamydomonas |
| | | | | | | | reinhardtii >gi\|18145\|emb\|CAA37638.1\| (X53574) |
| | | | | | | | putative protein has homology to G protein beta |
| | | | | | | | subunit [Ch |
| 941 | LIB3602-106-Q1-K1-C5 | 121026 | BLASTX | 160 | 1.00E−12 | 85 | GUANINE NUCLEOTIDE-BINDING PROTEIN |
| | | | | | | | BETA SUBUNIT-LIKE PROTEIN |
| | | | | | | | >gi\|99395\|pir\|\|S11904 GTP-binding regulatory |
| | | | | | | | protein beta chain homolog - Chlamydomonas |
| | | | | | | | reinhardtii >gi\|18145\|emb\|CAA37638.1\| (X53574) |
| | | | | | | | putative protein has homology to G protein beta |
| | | | | | | | subunit [Ch |
| 942 | LIB3602-044-Q6-K1-F8 | 4506697 | BLASTX | 475 | 1.00E−47 | 78 | ribosomal protein S20 |
| | | | | | | | >gi\|133875\|sp\|P17075\|RS20_HUMAN 40S |
| | | | | | | | RIBOSOMAL PROTEIN S20 |
| | | | | | | | >gi\|70925\|pir\|\|R3RT20 ribosomal protein S20, |
| | | | | | | | cytosolic - rat >gi\|423075\|pir\|\|S33710 ribosomal |
| | | | | | | | protein S20, cytosolic - human |
| | | | | | | | >gi\|57720\|emb\|CAA35917.1\| (X51537) ribosomal |
| | | | | | | | prote |
| 943 | LIB3602-085-Q6-K1-B1 | 4506697 | BLASTX | 475 | 1.00E−47 | 78 | ribosomal protein S20 |
| | | | | | | | >gi\|133875\|sp\|P17075\|RS20_HUMAN 40S |
| | | | | | | | RIBOSOMAL PROTEIN S20 |
| | | | | | | | >gi\|70925\|pir\|\|R3RT20 ribosomal protein S20, |
| | | | | | | | cytosolic - rat >gi\|423075\|pir\|\|S33710 ribosomal |
| | | | | | | | protein S20, cytosolic - human |
| | | | | | | | >gi\|57720\|emb\|CAA35917.1\| (X51537) ribosomal |
| | | | | | | | prote |
| 944 | LIB3602-042-Q6-K1-D2 | 4506697 | BLASTX | 475 | 2.00E−47 | 78 | ribosomal protein S20 |
| | | | | | | | >gi\|133875\|sp\|P17075\|RS20_HUMAN 40S |
| | | | | | | | RIBOSOMAL PROTEIN S20 |
| | | | | | | | >gi\|70925\|pir\|\|R3RT20 ribosomal protein S20, |
| | | | | | | | cytosolic - rat >gi\|423075\|pir\|\|S33710 ribosomal |
| | | | | | | | protein S20, cytosolic - human |
| | | | | | | | >gi\|57720\|emb\|CAA35917.1\| (X51537) ribosomal |
| | | | | | | | prote |
| 945 | LIB3602-027-Q6-K1-G1 | 4506697 | BLASTX | 475 | 2.00E−47 | 78 | ribosomal protein S20 |
| | | | | | | | >gi\|133875\|sp\|P17075\|RS20_HUMAN 40S |
| | | | | | | | RIBOSOMAL PROTEIN S20 |
| | | | | | | | >gi\|70925\|pir\|\|R3RT20 ribosomal protein S20, |
| | | | | | | | cytosolic - rat >gi\|423075\|pir\|\|S33710 ribosomal |
| | | | | | | | protein S20, cytosolic - human |
| | | | | | | | >gi\|57720\|emb\|CAA35917.1\| (X51537) ribosomal |
| | | | | | | | prote |
| 946 | LIB3602-007-Q1-K1-A7 | 4506697 | BLASTX | 472 | 3.00E−47 | 84 | ribosomal protein S20 |
| | | | | | | | >gi\|133875\|sp\|P17075\|RS20_HUMAN 40S |
| | | | | | | | RIBOSOMAL PROTEIN S20 |
| | | | | | | | >gi\|70925\|pir\|\|R3RT20 ribosomal protein S20, |
| | | | | | | | cytosolic - rat >gi\|423075\|pir\|\|S33710 ribosomal |
| | | | | | | | protein S20, cytosolic - human |
| | | | | | | | >gi\|57720\|emb\|CAA35917.1\| (X51537) ribosomal |
| | | | | | | | prote |
| 947 | LIB3602-022-Q6-K1-C2 | 4506697 | BLASTX | 451 | 7.00E−45 | 76 | ribosomal protein S20 |
| | | | | | | | >gi\|133875\|sp\|P17075\|RS20_HUMAN 40S |
| | | | | | | | RIBOSOMAL PROTEIN S20 |
| | | | | | | | >gi\|70925\|pir\|\|R3RT20 ribosomal protein S20, |
| | | | | | | | cytosolic - rat >gi\|423075\|pir\|\|S33710 ribosomal |
| | | | | | | | protein S20, cytosolic - human |
| | | | | | | | >gi\|57720\|emb\|CAA35917.1\| (X51537) ribosomal |
| | | | | | | | prote |
| 948 | LIB3602-003-Q1-K1-F12 | 4506697 | BLASTX | 450 | 1.00E−44 | 74 | ribosomal protein S20 |
| | | | | | | | >gi\|133875\|sp\|P17075\|RS20_HUMAN 40S |
| | | | | | | | RIBOSOMAL PROTEIN S20 |
| | | | | | | | >gi\|70925\|pir\|\|R3RT20 ribosomal protein S20, |
| | | | | | | | cytosolic - rat >gi\|423075\|pir\|\|S33710 ribosomal |
| | | | | | | | protein S20, cytosolic - human |
| | | | | | | | >gi\|57720\|emb\|CAA35917.1\| (X51537) ribosomal |
| | | | | | | | prote |
| 949 | LIB3602-108-Q1-K1-G4 | 4506697 | BLASTX | 311 | 3.00E−42 | 82 | ribosomal protein S20 |
| | | | | | | | >gi\|133875\|sp\|P17075\|RS20_HUMAN 40S |
| | | | | | | | RIBOSOMAL PROTEIN S20 |
| | | | | | | | >gi\|70925\|pir\|\|R3RT20 ribosomal protein S20, |
| | | | | | | | cytosolic - rat >gi\|423075\|pir\|\|S33710 ribosomal |
| | | | | | | | protein S20, cytosolic - human |
| | | | | | | | >gi\|57720\|emb\|CAA35917.1\| (X51537) ribosomal |
| | | | | | | | prote |
| 950 | LIB3602-007-Q1-K1-C3 | 4506697 | BLASTX | 428 | 5.00E−42 | 73 | ribosomal protein S20 |
| | | | | | | | >gi\|133875\|sp\|P17075\|RS20_HUMAN 40S |
| | | | | | | | RIBOSOMAL PROTEIN S20 |
| | | | | | | | >gi\|70925\|pir\|\|R3RT20 ribosomal protein S20, |
| | | | | | | | cytosolic - rat >gi\|423075\|pir\|\|S33710 ribosomal |
| | | | | | | | protein S20, cytosolic - human |
| | | | | | | | >gi\|57720\|emb\|CAA35917.1\| (X51537) ribosomal |
| | | | | | | | prote |
| 951 | LIB3602-078-Q6-K6-F6 | 6521012 | BLASTX | 634 | 4.00E−66 | 82 | (AB031739) cytoplasmic ribosomal protein S13 |
| | | | | | | | [Arabidopsis thaliana] |
| 952 | LIB3602-089-Q6-K6-C6 | 6521012 | BLASTX | 631 | 9.00E−66 | 82 | (AB031739) cytoplasmic ribosomal protein S13 |
| | | | | | | | [Arabidopsis thaliana] |
| 953 | LIB3602-083-Q6-K6-H6 | 6521012 | BLASTX | 538 | 8.00E−61 | 78 | (AB031739) cytoplasmic ribosomal protein S13 |
| | | | | | | | [Arabidopsis thaliana] |
| 954 | LIB3602-010-Q6-K1-A4 | 6521012 | BLASTX | 540 | 4.00E−55 | 82 | (AB031739) cytoplasmic ribosomal protein S13 |
| | | | | | | | [Arabidopsis thaliana] |
| 955 | LIB3602-028-Q6-K1-F9 | 6521012 | BLASTX | 538 | 7.00E−55 | 82 | (AB031739) cytoplasmic ribosomal protein S13 |
| | | | | | | | [Arabidopsis thaliana] |
| 956 | LIB3602-010-Q6-K1-A5 | 6521012 | BLASTX | 511 | 1.00E−51 | 84 | (AB031739) cytoplasmic ribosomal protein S13 |
| | | | | | | | [Arabidopsis thaliana] |
| 957 | LIB3602-105-Q1-K1-G3 | 6521012 | BLASTX | 483 | 2.00E−48 | 86 | (AB031739) cytoplasmic ribosomal protein S13 |
| | | | | | | | [Arabidopsis thaliana] |
| 958 | LIB3602-118-Q1-K1-A1 | 6521012 | BLASTX | 311 | 8.00E−43 | 83 | (AB031739) cytoplasmic ribosomal protein S13 |
| | | | | | | | [Arabidopsis thaliana] |
| 959 | LIB3602-023-Q6-K1-B5 | 6521012 | BLASTX | 218 | 2.00E−17 | 71 | (AB031739) cytoplasmic ribosomal protein S13 |
| | | | | | | | [Arabidopsis thaliana] |
| 960 | LIB3602-078-Q6-K6-H9 | 1351014 | BLASTX | 655 | 1.00E−68 | 68 | 40S RIBOSOMAL PROTEIN S8 |
| | | | | | | | >gi\|7440236\|pir\|\|T04082 probable ribosomal protein |
| | | | | | | | S8 - rice >gi\|968902\|dbj\|BAA07207.1\| (D38010) |
| | | | | | | | ribosomal protein S8 [Oryza sativa] |
| 961 | LIB3602-039-Q6-K1-A12 | 1351014 | BLASTX | 572 | 7.00E−59 | 65 | 40S RIBOSOMAL PROTEIN S8 |
| | | | | | | | >gi\|7440236\|pir\|\|T04082 probable ribosomal protein |
| | | | | | | | S8 - rice >gi\|968902\|dbj\|BAA07207.1\| (D38010) |
| | | | | | | | ribosomal protein S8 [Oryza sativa] |
| 962 | LIB3602-010-Q6-K1-D8 | 1351014 | BLASTX | 518 | 1.00E−52 | 66 | 40S RIBOSOMAL PROTEIN S8 |
| | | | | | | | >gi\|7440236\|pir\|\|T04082 probable ribosomal protein |
| | | | | | | | S8 - rice >gi\|968902\|dbj\|BAA07207.1\| (D38010) |
| | | | | | | | ribosomal protein S8 [Oryza sativa] |
| 963 | LIB3602-003-Q1-K1-E12 | 1351014 | BLASTX | 515 | 3.00E−52 | 64 | 40S RIBOSOMAL PROTEIN S8 |
| | | | | | | | >gi\|7440236\|pir\|\|T04082 probable ribosomal protein |
| | | | | | | | S8 - rice >gi\|968902\|dbj\|BAA07207.1\| (D38010) |
| | | | | | | | ribosomal protein S8 [Oryza sativa] |
| 964 | LIB3602-083-Q6-K6-H5 | 1351014 | BLASTX | 509 | 2.00E−51 | 63 | 40S RIBOSOMAL PROTEIN S8 |
| | | | | | | | >gi\|7440236\|pir\|\|T04082 probable ribosomal protein |
| | | | | | | | S8 - rice >gi\|968902\|dbj\|BAA07207.1\| (D38010) |
| | | | | | | | ribosomal protein S8 [Oryza sativa] |
| 965 | LIB3602-109-Q1-K1-H5 | 1351014 | BLASTX | 334 | 1.00E−44 | 61 | 40S RIBOSOMAL PROTEIN S8 |
| | | | | | | | >gi\|7440236\|pir\|\|T04082 probable ribosomal protein |
| | | | | | | | S8 - rice >gi\|968902\|d$$ BAA07207.1\| (D38010) |
| | | | | | | | ribosomal protein S8 [Oryza sativa] |
| 966 | LIB3602-014-Q6-K1-C9 | 1351014 | BLASTX | 257 | 5.00E−22 | 55 | 40S RIBOSOMAL PROTEIN S8 |
| | | | | | | | >gi\|7440236\|pir\|\|T04082 probable ribosomal protein |
| | | | | | | | S8 - rice >gi\|968902\|dbj\|BAA07207.1\| (D38010) |
| | | | | | | | ribosomal protein S8 [Oryza sativa] |
| 967 | LIB3602-037-Q6-K1-B1 | 1351014 | BLASTX | 242 | 8.00E−21 | 68 | 40S RIBOSOMAL PROTEIN S8 |
| | | | | | | | >gi\|7440236\|pir\|\|T04082 probable ribosomal protein |
| | | | | | | | S8 - rice >gi\|968902\|dbj\|BAA07207.1\| (D38010) |
| | | | | | | | ribosomal protein S8 [Oryza sativa] |
| 968 | LIB3602-011-Q6-K1-D7 | 1351014 | BLASTX | 225 | 3.00E−18 | 69 | 40S RIBOSOMAL PROTEIN S8 |
| | | | | | | | >gi\|7440236\|pir\|\|T04082 probable ribosomal protein |
| | | | | | | | S8 - rice >gi\|968902\|dbj\|BAA07207.1\| (D38010) |
| | | | | | | | ribosomal protein S8 [Oryza sativa] |
| 969 | LIB3602-095-Q6-K6-F2 | 6174958 | BLASTX | 778 | 6.00E−83 | 72 | 60S RIBOSOMAL PROTEIN L7 |
| | | | | | | | >gi\|7440720\|pir\|\|T00692 ribosomal protein L7, |
| | | | | | | | cytosolic - Arabidopsis thaliana |
| | | | | | | | >gi\|3212879\|gb\|AAC23430.1\| (AC004005) 60S |
| | | | | | | | ribosomal protein L7 [Arabidopsis thaliana] |
| 970 | LIB3602-089-Q6-K6-F2 | 6174958 | BLASTX | 753 | 5.00E−80 | 71 | 60S RIBOSOMAL PROTEIN L7 |
| | | | | | | | >gi\|7440720\|pir\|\|T00692 ribosomal protein L7, |
| | | | | | | | cytosolic - Arabidopsis thaliana |
| | | | | | | | >gi\|3212879\|gb\|AAC23430.1\| (AC004005) 60S |
| | | | | | | | ribosomal protein L7 [Arabidopsis thaliana] |
| 971 | LIB3602-076-Q6-K6-F4 | 6174958 | BLASTX | 677 | 4.00E−71 | 67 | 60S RIBOSOMAL PROTEIN L7 |
| | | | | | | | >gi\|7440720\|pir\|\|T00692 ribosomal protein L7, |
| | | | | | | | cytosolic - Arabidopsis thaliana |
| | | | | | | | >gi\|3212879\|gb\|AAC23430.1\| (AC004005) 60S |
| | | | | | | | ribosomal protein L7 [Arabidopsis thaliana] |
| 972 | LIB3602-044-Q6-K1-G11 | 6174958 | BLASTX | 633 | 4.00E−66 | 75 | 60S RIBOSOMAL PROTEIN L7 |
| | | | | | | | >gi\|7440720\|pir\|\|T00692 ribosomal protein L7, |
| | | | | | | | cytosolic - Arabidopsis thaliana |
| | | | | | | | >gi\|3212879\|gb\|AAC23430.1\| (AC004005) 60S |
| | | | | | | | ribosomal protein L7 [Arabidopsis thaliana] |
| 973 | LIB3602-006-Q1-K1-C1 | 6174958 | BLASTX | 632 | 7.00E−66 | 67 | 60S RIBOSOMAL PROTEIN L7 |
| | | | | | | | >gi\|7440720\|pir\|\|T00692 ribosomal protein L7, |
| | | | | | | | cytosolic - Arabidopsis thaliana |
| | | | | | | | >gi\|3212879\|gb\|AAC23430.1\| (AC004005) 60S |
| | | | | | | | ribosomal protein L7 [Arabidopsis thaliana] |
| 974 | LIB3602-043-Q6-K1-D11 | 6174958 | BLASTX | 622 | 1.00E−64 | 74 | 60S RIBOSOMAL PROTEIN L7 |
| | | | | | | | >gi\|7440720\|pir\|\|T00692 ribosomal protein L7, |
| | | | | | | | cytosolic - Arabidopsis thaliana |
| | | | | | | | >gi\|3212879\|gb\|AAC23430.1\| (AC004005) 60S |
| | | | | | | | ribosomal protein L7 [Arabidopsis thaliana] |
| 975 | LIB3602-027-Q6-K1-E10 | 6174958 | BLASTX | 609 | 3.00E−63 | 75 | 60S RIBOSOMAL PROTEIN L7 |
| | | | | | | | >gi\|7440720\|pir\|\|T00692 ribosomal protein L7, |
| | | | | | | | cytosolic - Arabidopsis thaliana |
| | | | | | | | >gi\|3212879\|gb\|AAC23430.1\| (AC004005) 60S |
| | | | | | | | ribosomal protein L7 [Arabidopsis thaliana] |
| 976 | LIB3602-060-Q6-K1-H2 | 6174958 | BLASTX | 598 | 6.00E−62 | 75 | 60S RIBOSOMAL PROTEIN L7 |
| | | | | | | | >gi\|7440720\|pir\|\|T00692 ribosomal protein L7, |
| | | | | | | | cytosolic - Arabidopsis thaliana |
| | | | | | | | >gi\|3212879\|gb\|AAC23430.1\| (AC004005) 60S |
| | | | | | | | ribosomal protein L7 [Arabidopsis thaliana] |
| 977 | LIB3602-054-Q6-K1-F11 | 6174958 | BLASTX | 206 | 5.00E−16 | 33 | 60S RIBOSOMAL PROTEIN L7 |
| | | | | | | | >gi\|7440720\|pir\|\|T00692 ribosomal protein L7, |
| | | | | | | | cytosolic - Arabidopsis thaliana |
| | | | | | | | >gi\|3212879\|gb\|AAC23430.1\| (AC004005) 60S |
| | | | | | | | ribosomal protein L7 [Arabidopsis thaliana] |
| 978 | LIB3602-046-Q6-K1-A11 | 1173187 | BLASTX | 677 | 3.00E−71 | 90 | 40S RIBOSOMAL PROTEIN S23 (S12) |
| | | | | | | | >gi\|1362041\|pir\|\|S56673 ribosomal protein S23.e, |
| | | | | | | | cytosolic (clone RJ3) - garden strawberry |
| | | | | | | | >gi\|643074\|gb\|AAA79921.1\| (U19940) putative 40S |
| | | | | | | | ribosomal protein s12 [Fragaria x ananassa] |
| 979 | LIB3602-030-Q6-K1-C8 | 1173187 | BLASTX | 677 | 3.00E−71 | 90 | 40S RIBOSOMAL PROTEIN S23 (S12) |
| | | | | | | | >gi\|1362041\|pir\|\|S56673 ribosomal protein S23.e, |
| | | | | | | | cytosolic (clone RJ3) - garden strawberry |
| | | | | | | | >gi\|643074\|gb\|AAA79921.1\| (U19940) putative 40S |
| | | | | | | | ribosomal protein s12 [Fragaria x ananassa] |
| 980 | LIB3602-039-Q6-K1-E1 | 1173187 | BLASTX | 677 | 4.00E−71 | 90 | 40S RIBOSOMAL PROTEIN S23 (S12) |
| | | | | | | | >gi\|1362041\|pir\|\|S56673 ribosomal protein S23.e, |
| | | | | | | | cytosolic (clone RJ3) - garden strawberry |
| | | | | | | | >gi\|643074\|gb\|AAA79921.1\| (U19940) putative 40S |
| | | | | | | | ribosomal protein s12 [Fragaria x ananassa] |
| 981 | LIB3602-067-Q1-K1-F6 | 1173187 | BLASTX | 674 | 9.00E−71 | 89 | 40S RIBOSOMAL PROTEIN S23 (S12) |
| | | | | | | | >gi\|1362041\|pir\|\|S56673 ribosomal protein S23.e, |
| | | | | | | | cytosolic (clone RJ3) - garden strawberry |
| | | | | | | | >gi\|643074\|gb\|AAA79921.1\| (U19940) putative 40S |
| | | | | | | | ribosomal protein s12 [Fragaria x ananassa] |
| 982 | LIB3602-100-Q1-K1-F9 | 1173187 | BLASTX | 488 | 7.00E−66 | 94 | 40S RIBOSOMAL PROTEIN S23 (S12) |
| | | | | | | | >gi\|1362041\|pir\|\|S56673 ribosomal protein S23.e, |
| | | | | | | | cytosolic (clone RJ3) - garden strawberry |
| | | | | | | | >gi\|643074\|gb\|AAA79921.1\| (U19940) putative 40S |
| | | | | | | | ribosomal protein s12 [Fragaria x ananassa] |
| 983 | LIB3602-037-Q6-K1-B2 | 1173187 | BLASTX | 582 | 3.00E−60 | 90 | 40S RIBOSOMAL PROTEIN S23 (S12) |
| | | | | | | | >gi\|1362041\|pir\|\|S56673 ribosomal protein S23.e, |
| | | | | | | | cytosolic (clone RJ3) - garden strawberry |
| | | | | | | | >gi\|643074\|gb\|AAA79921.1\| (U19940) putative 40S |
| | | | | | | | ribosomal protein s12 [Fragaria x ananassa] |
| 984 | LIB3602-022-Q6-K1-C8 | 1173187 | BLASTX | 582 | 4.00E−60 | 90 | 40S RIBOSOMAL PROTEIN S23 (S12) |
| | | | | | | | >gi\|1362041\|pir\|\|S56673 ribosomal protein S23.e, |
| | | | | | | | cytosolic (clone RJ3) - garden strawberry |
| | | | | | | | >gi\|643074\|gb\|AAA79921.1\| (U19940) putative 40S |
| | | | | | | | ribosomal protein s12 [Fragaria x ananassa] |
| 985 | LIB3602-046-Q6-K1-A12 | 1173187 | BLASTX | 536 | 7.00E−55 | 87 | 40S RIBOSOMAL PROTEIN S23 (S12) |
| | | | | | | | >gi\|1362041\|pir\|\|S56673 ribosomal protein S23.e, |
| | | | | | | | cytosolic (clone RJ3) - garden strawberry |
| | | | | | | | >gi\|643074\|gb\|AAA79921.1\| (U19940) putative 40S |
| | | | | | | | ribosomal protein s12 [Fragaria x ananassa] |
| 986 | LIB3602-021-Q6-K1-D3 | 1173187 | BLASTX | 344 | 1.00E−32 | 87 | 40S RIBOSOMAL PROTEIN S23 (S12) |
| | | | | | | | >gi\|1362041\|pir\|\|S56673 ribosomal protein S23.e, |
| | | | | | | | cytosolic (clone RJ3) - garden strawberry |
| | | | | | | | >gi\|643074\|gb\|AAA79921.1\| (U19940) putative 40S |
| | | | | | | | ribosomal protein s12 [Fragaria x ananassa] |
| 987 | LIB3602-068-Q1-K1-D1 | 6671950 | BLASTX | 439 | 2.00E−43 | 90 | (AC016795) putative 40S ribosomal protein S5 |
| | | | | | | | [Arabidopsis thaliana] |
| 988 | LIB3602-081-Q6-K6-G1 | 6671950 | BLASTX | 405 | 2.00E−39 | 96 | (AC016795) putative 40S ribosomal protein S5 |
| | | | | | | | [Arabidopsis thaliana] |
| 989 | LIB3602-086-Q6-K1-E5 | 6671950 | BLASTX | 405 | 2.00E−39 | 96 | (AC016795) putative 40S ribosomal protein S5 |
| | | | | | | | [Arabidopsis thaliana] |
| 990 | LIB3602-023-Q6-K1-G12 | 6671950 | BLASTX | 376 | 5.00E−36 | 96 | (AC016795) putative 40S ribosomal protein S5 |
| | | | | | | | [Arabidopsis thaliana] |
| 991 | LIB3602-117-Q1-K1-B8 | 6671950 | BLASTX | 190 | 1.00E−30 | 97 | (AC016795) putative 40S ribosomal protein S5 |
| | | | | | | | [Arabidopsis thaliana] |
| 992 | LIB3602-055-Q6-K1-C8 | 6671950 | BLASTX | 318 | 3.00E−29 | 91 | (AC016795) putative 40S ribosomal protein S5 |
| | | | | | | | [Arabidopsis thaliana] |
| 993 | LIB3602-057-Q6-K1-E12 | 6671950 | BLASTX | 186 | 2.00E−28 | 90 | (AC016795) putative 40S ribosomal protein S5 |
| | | | | | | | [Arabidopsis thaliana] |
| 994 | LIB3602-026-Q6-K1-F5 | 6671950 | BLASTX | 240 | 1.00E−21 | 84 | (AC016795) putative 40S ribosomal protein S5 |
| | | | | | | | [Arabidopsis thaliana] |
| 995 | LIB3602-007-Q1-K1-B2 | 6671950 | BLASTX | 209 | 5.00E−17 | 89 | (AC016795) putative 40S ribosomal protein S5 |
| | | | | | | | [Arabidopsis thaliana] |
| 996 | LIB3602-036-Q6-K1-A8 | 99487 | BLASTX | 449 | 2.00E−44 | 60 | chlorophyll a/b-binding protein (clone pINEab 43) - |
| | | | | | | | Scotch pine >gi\|20794\|emb\|CAA41407.1\| (X58517) |
| | | | | | | | Type III chlorophyll a/b-binding protein [Pinus |
| | | | | | | | sylvestris] |
| 997 | LIB3602-068-Q1-K1-B5 | 99487 | BLASTX | 398 | 2.00E−38 | 60 | chlorophyll a/b-binding protein (clone pINEab 43) - |
| | | | | | | | Scotch pine >gi\|20794\|emb\|CAA41407.1\| (X58517) |
| | | | | | | | Type III chlorophyll a/b-binding protein [Pinus |
| | | | | | | | sylvestris] |
| 998 | LIB3602-052-Q6-K1-G12 | 99487 | BLASTX | 381 | 1.00E−36 | 58 | chlorophyll a/b-binding protein (clone pINEab 43) - |
| | | | | | | | Scotch pine >gi\|20794\|emb\|CAA41407.1\| (X58517) |
| | | | | | | | Type III chlorophyll a/b-binding protein [Pinus |
| | | | | | | | sylvestris] |
| 999 | LIB3602-048-Q6-K1QA- | 99487 | BLASTX | 348 | 8.00E−33 | 61 | chlorophyll a/b-binding protein (clone pINEab 43) - |
| | H11 | | | | | | Scotch pine >gi\|20794\|emb\|CAA41407.1\| (X58517) |
| | | | | | | | Type III chlorophyll a/b-binding protein [Pinus |
| | | | | | | | sylvestris] |
| 1000 | LIB3602-020-Q6-K1-F5 | 99487 | BLASTX | 343 | 4.00E−32 | 61 | chlorophyll a/b-binding protein (clone pINEab 43) - |
| | | | | | | | Scotch pine >gi\|20794\|emb\|CAA41407.1\| (X58517) |
| | | | | | | | Type III chlorophyll a/b-binding protein [Pinus |
| | | | | | | | sylvestris] |
| 1001 | LIB3602-112-Q1-K1-F10 | 99487 | BLASTX | 329 | 3.00E−30 | 62 | chlorophyll a/b-binding protein (clone pINEab 43) - |
| | | | | | | | Scotch pine >gi\|20794\|emb\|CAA41407.1\| (X58517) |
| | | | | | | | Type III chlorophyll a/b-binding protein [Pinus |
| | | | | | | | sylvestris] |
| 1002 | LIB3602-089-Q6-K6-F10 | 99487 | BLASTX | 320 | 3.00E−29 | 60 | chlorophyll a/b-binding protein (clone pINEab 43) - |
| | | | | | | | Scotch pine >gi\|20794\|emb\|CAA41407.1\| (X58517) |
| | | | | | | | Type III chlorophyll a/b-binding protein [Pinus |
| | | | | | | | sylvestris] |
| 1003 | LIB3602-021-Q6-K1-H2 | 99487 | BLASTX | 157 | 1.00E−10 | 58 | chlorophyll a/b-binding protein (clone pINEab 43) - |
| | | | | | | | Scotch pine >gi\|20794\|emb\|CAA41407.1\| (X58517) |
| | | | | | | | Type III chlorophyll a/b-binding protein [Pinus |
| | | | | | | | sylvestris] |
| 1004 | LIB3602-118-Q1-K1-C11 | 99487 | BLASTX | 157 | 3.00E−10 | 58 | chlorophyll a/b-binding protein (clone pINEab 43) - |
| | | | | | | | Scotch pine >gi\|20794\|emb\|CAA41407.1\| (X58517) |
| | | | | | | | Type III chlorophyll a/b-binding protein [Pinus |
| | | | | | | | sylvestris] |
| 1005 | LIB3602-030-Q6-K1-B7 | 3953473 | BLASTX | 271 | 1.00E−23 | 54 | (AC002328) F22O2.18 [Arabidopsis thaliana] |
| | | | | | | | >gi\|5734520\|emb\|CAB52748.1\| (AJ245630) |
| | | | | | | | photosystem I subunit V precursor [Arabidopsis |
| | | | | | | | thaliana] |
| 1006 | LIB3602-083-Q6-K6-C9 | 3953473 | BLASTX | 271 | 1.00E−23 | 54 | (AC002328) F22O2.18 [Arabidopsis thaliana] |
| | | | | | | | >gi\|5734520\|emb\|CAB52748.1\| (AJ245630) |
| | | | | | | | photosystem I subunit V precursor [Arabidopsis |
| | | | | | | | thaliana] |
| 1007 | LIB3602-054-Q6-K1-G2 | 3953473 | BLASTX | 271 | 1.00E−23 | 54 | (AC002328) F22O2.18 [Arabidopsis thaliana] |
| | | | | | | | >gi\|5734520\|emb\|CAB52748.1\| (AJ245630) |
| | | | | | | | photosystem I subunit V precursor [Arabidopsis |
| | | | | | | | thaliana] |
| 1008 | LIB3602-109-Q1-K1-C9 | 3953473 | BLASTX | 265 | 7.00E−23 | 59 | (AC002328) F22O2.18 [Arabidopsis thaliana] |
| | | | | | | | >gi\|5734520\|emb\|CAB52748.1\| (AJ245630) |
| | | | | | | | photosystem I subunit V precursor [Arabidopsis |
| | | | | | | | thaliana] |
| 1009 | LIB3602-053-Q6-K1-D12 | 3953473 | BLASTX | 264 | 1.00E−22 | 53 | (AC002328) F22O2.18 [Arabidopsis thaliana] |
| | | | | | | | >gi\|5734520\|emb\|CAB52748.1\| (AJ245630) |
| | | | | | | | photosystem I subunit V precursor [Arabidopsis |
| | | | | | | | thaliana] |
| 1010 | LIB3602-079-Q6-K6-A1 | 3953473 | BLASTX | 240 | 4.00E−20 | 57 | (AC002328) F22O2.18 [Arabidopsis thaliana] |
| | | | | | | | >gi\|5734520\|emb\|CAB52748.1\| (AJ245630) |
| | | | | | | | photosystem I subunit V precursor [Arabidopsis |
| | | | | | | | thaliana] |
| 1011 | LIB3602-067-Q1-K1-B12 | 3953473 | BLASTX | 216 | 2.00E−17 | 60 | (AC002328) F22O2.18 [Arabidopsis thaliana] |
| | | | | | | | >gi\|5734520\|emb\|CAB52748.1\| (AJ245630) |
| | | | | | | | photosystem I subunit V precursor [Arabidopsis |
| | | | | | | | thaliana] |
| 1012 | LIB3602-087-Q6-K1-E2 | 3953473 | BLASTX | 216 | 2.00E−17 | 60 | (AC002328) F22O2.18 [Arabidopsis thaliana] |
| | | | | | | | >gi\|5734520\|emb\|CAB52748.1\| (AJ245630) |
| | | | | | | | photosystem I subunit V precursor [Arabidopsis |
| | | | | | | | thaliana] |
| 1013 | LIB3602-056-Q6-K1-F2 | 3953473 | BLASTX | 145 | 2.00E−13 | 57 | (AC002328) F22O2.18 [Arabidopsis thaliana] |
| | | | | | | | >gi\|5734520\|emb\|CAB52748.1\| (AJ245630) |
| | | | | | | | photosystem I subunit V precursor [Arabidopsis |
| | | | | | | | thaliana] |
| 1014 | LIB3602-054-Q6-K1-F10 | 131395 | BLASTX | 383 | 8.00E−37 | 59 | OXYGEN-EVOLVING ENHANCER PROTEIN 3 |
| | | | | | | | PRECURSOR (OEE3) >gi\|81254\|pir\|\|S05509 |
| | | | | | | | photosystem II oxygen-evolving complex protein 3 |
| | | | | | | | precursor - Chlamydomonas reinhardtii |
| | | | | | | | >gi\|18164\|emb\|CAA32061.1\| (X13832) OEE3 |
| | | | | | | | precursor protein [Chlamydomonas reinhardtii] |
| | | | | | | | >gi\|2286 |
| 1015 | LIB3602-071-Q1-K1-G3 | 131395 | BLASTX | 260 | 7.00E−30 | 55 | OXYGEN-EVOLVING ENHANCER PROTEIN 3 |
| | | | | | | | PRECURSOR (OEE3) >gi\|81254\|pir\|\|S05509 |
| | | | | | | | photosystem II oxygen-evolving complex protein 3 |
| | | | | | | | precursor - Chlamydomonas reinhardtii |
| | | | | | | | >gi\|18164\|emb\|CAA32061.1\| (X13832) OEE3 |
| | | | | | | | precursor protein [Chlamydomonas reinhardtii] |
| | | | | | | | >gi\|2286 |
| 1016 | LIB3602-102-Q1-K1-F3 | 131395 | BLASTX | 297 | 8.00E−27 | 57 | OXYGEN-EVOLVING ENHANCER PROTEIN 3 |
| | | | | | | | PRECURSOR (OEE3) >gi\|81254\|pir\|\|S05509 |
| | | | | | | | photosystem II oxygen-evolving complex protein 3 |
| | | | | | | | precursor - Chlamydomonas reinhardtii |
| | | | | | | | >gi\|18164\|emb\|CAA32061.1\| (X13832) OEE3 |
| | | | | | | | precursor protein [Chlamydomonas reinhardtii] |
| | | | | | | | >gi\|2286 |
| 1017 | LIB3602-016-Q6-K1-C7 | 131395 | BLASTX | 292 | 4.00E−26 | 57 | OXYGEN-EVOLVING ENHANCER PROTEIN 3 |
| | | | | | | | PRECURSOR (OEE3) >gi\|81254\|pir\|\|S05509 |
| | | | | | | | photosystem II oxygen-evolving complex protein 3 |
| | | | | | | | precursor - Chlamydomonas reinhardtii |
| | | | | | | | >gi\|18164\|emb\|CAA32061.1\| (X13832) OEE3 |
| | | | | | | | precursor protein [Chlamydomonas reinhardtii] |
| | | | | | | | >gi\|2286 |
| 1018 | LIB3602-073-Q1-K1-H1 | 131395 | BLASTX | 260 | 3.00E−22 | 55 | OXYGEN-EVOLVING ENHANCER PROTEIN 3 |
| | | | | | | | PRECURSOR (OEE3) >gi\|81254\|pir\|\|S05509 |
| | | | | | | | photosystem II oxygen-evolving complex protein 3 |
| | | | | | | | precursor - Chlamydomonas reinhardtii |
| | | | | | | | >gi\|18164\|emb\|CAA32061.1\| (X13832) OEE3 |
| | | | | | | | precursor protein [Chlamydomonas reinhardtii] |
| | | | | | | | >gi\|2286 |
| 1019 | LIB3602-109-Q1-K1-D7 | 131395 | BLASTX | 148 | 7.00E−16 | 60 | OXYGEN-EVOLVING ENHANCER PROTEIN 3 |
| | | | | | | | PRECURSOR (OEE3) >gi\|81254\|pir\|\|S05509 |
| | | | | | | | photosystem II oxygen-evolving complex protein 3 |
| | | | | | | | precursor - Chlamydomonas reinhardtii |
| | | | | | | | >gi\|18164\|emb\|CAA32061.1\| (X13832) OEE3 |
| | | | | | | | precursor protein [Chlamydomonas reinhardtii] |
| | | | | | | | >gi\|2286 |
| 1020 | LIB3602-119-Q1-K1-E1 | 131395 | BLASTX | 199 | 2.00E−15 | 54 | OXYGEN-EVOLVING ENHANCER PROTEIN 3 |
| | | | | | | | PRECURSOR (OEE3) >gi\|81254\|pir\|\|S05509 |
| | | | | | | | photosystem II oxygen-evolving complex protein 3 |
| | | | | | | | precursor - Chlamydomonas reinhardtii |
| | | | | | | | >gi\|18164\|emb\|CAA32061.1\| (X13832) OEE3 |
| | | | | | | | precursor protein [Chlamydomonas reinhardtii] |
| | | | | | | | >gi\|2286 |
| 1021 | LIB3602-092-Q6-K6-G4 | 131395 | BLASTX | 199 | 5.00E−15 | 54 | OXYGEN-EVOLVING ENHANCER PROTEIN 3 |
| | | | | | | | PRECURSOR (OEE3) >gi\|81254\|pir\|\|S05509 |
| | | | | | | | photosystem II oxygen-evolving complex protein 3 |
| | | | | | | | precursor - Chlamydomonas reinhardtii |
| | | | | | | | >gi\|18164\|emb\|CAA32061.1\| (X13832) OEE3 |
| | | | | | | | precursor protein [Chlamydomonas reinhardtii] |
| | | | | | | | >gi\|2286 |
| 1022 | LIB3602-054-Q6-K1-F5 | 131395 | BLASTX | 145 | 6.00E−09 | 48 | OXYGEN-EVOLVING ENHANCER PROTEIN 3 |
| | | | | | | | PRECURSOR (OEE3) >gi\|81254\|pir\|\|S05509 |
| | | | | | | | photosystem II oxygen-evolving complex protein 3 |
| | | | | | | | precursor - Chlamydomonas reinhardtii |
| | | | | | | | >gi\|18164\|emb\|CAA32061.1\| (X13832) OEE3 |
| | | | | | | | precursor protein [Chlamydomonas reinhardtii] |
| | | | | | | | >gi\|2286 |
| 1023 | LIB3602-047-Q6-K1-F1 | 7413571 | BLASTX | 505 | 4.00E−51 | 94 | (AL162973) putative protein [Arabidopsis thaliana] |
| 1024 | LIB3602-084-Q6-K1-B10 | 7413571 | BLASTX | 455 | 1.00E−46 | 92 | (AL162973) putative protein [Arabidopsis thaliana] |
| 1025 | LIB3602-008-Q6-K1-C6 | 7413571 | BLASTX | 422 | 2.00E−41 | 92 | (AL162973) putative protein [Arabidopsis thaliana] |
| 1026 | LIB3602-083-Q6-K6-A7 | 7413571 | BLASTX | 413 | 3.00E−40 | 94 | (AL162973) putative protein [Arabidopsis thaliana] |
| 1027 | LIB3602-008-Q6-K1-C3 | 7413571 | BLASTX | 411 | 5.00E−40 | 90 | (AL162973) putative protein [Arabidopsis thaliana] |
| 1028 | LIB3602-091-Q6-K6-A3 | 7413571 | BLASTX | 299 | 8.00E−27 | 81 | (AL162973) putative protein [Arabidopsis thaliana] |
| 1029 | LIB3602-095-Q6-K6-A3 | 7413571 | BLASTX | 298 | 1.00E−26 | 91 | (AL162973) putative protein [Arabidopsis thaliana] |
| 1030 | LIB3602-094-Q6-K6-A3 | 7413571 | BLASTX | 213 | 5.00E−21 | 88 | (AL162973) putative protein [Arabidopsis thaliana] |
| 1031 | LIB3602-039-Q6-K1-E2 | 7413571 | BLASTX | 222 | 4.00E−18 | 48 | (AL162973) putative protein [Arabidopsis thaliana] |
| 1032 | LIB3602-113-Q1-K1-G7 | 5821436 | BLASTX | 721 | 3.00E−76 | 69 | (AB031546) chloroplast w6 desaturase |
| | | | | | | | [Chlamydomonas sp. W80] |
| 1033 | LIB3602-015-Q6-K1-H9 | 5821436 | BLASTX | 689 | 1.00E−72 | 65 | (AB031546) chloroplast w6 desaturase |
| | | | | | | | [Chlamydomonas sp. W80] |
| 1034 | LIB3602-108-Q1-K1-C5 | 5821436 | BLASTX | 616 | 4.00E−64 | 70 | (AB031546) chloroplast w6 desaturase |
| | | | | | | | [Chlamydomonas sp. W80] |
| 1035 | LIB3602-120-Q1-K1-G7 | 5821436 | BLASTX | 601 | 2.00E−62 | 72 | (AB031546) chloroplast w6 desaturase |
| | | | | | | | [Chlamydomonas sp. W80] |
| 1036 | LIB3602-113-Q1-K1-C2 | 5821436 | BLASTX | 560 | 2.00E−57 | 66 | (AB031546) chloroplast w6 desaturase |
| | | | | | | | [Chlamydomonas sp. W80] |
| 1037 | LIB3602-120-Q1-K1-C2 | 5821436 | BLASTX | 466 | 1.00E−46 | 66 | (AB031546) chloroplast w6 desaturase |
| | | | | | | | [Chlamydomonas sp. W80] |
| 1038 | LIB3602-092-Q6-K6-D2 | 5821436 | BLASTX | 424 | 2.00E−41 | 74 | (AB031546) chloroplast w6 desaturase |
| | | | | | | | [Chlamydomonas sp. W80] |
| 1039 | LIB3602-021-Q6-K1-E5 | 5821436 | BLASTX | 314 | 4.00E−29 | 69 | (AB031546) chloroplast w6 desaturase |
| | | | | | | | [Chlamydomonas sp. W80] |
| 1040 | LIB3602-104-Q1-K1-D3 | 1173209 | BLASTX | 554 | 1.00E−56 | 78 | 40S RIBOSOMAL PROTEIN S16 |
| | | | | | | | >gi\|541835\|pir\|\|S41193 ribosomal protein S16 |
| | | | | | | | protein - upland cotton |
| | | | | | | | >gi\|439654\|emb\|CAA53567.1\| (X75954) RS16 |
| | | | | | | | protein, 40S subunit [Gossypium hirsutum] |
| 1041 | LIB3602-051-Q6-K1-E2 | 1173209 | BLASTX | 538 | 6.00E−55 | 74 | 40S RIBOSOMAL PROTEIN S16 |
| | | | | | | | >gi\|541835\|pir\|\|S41193 ribosomal protein S16 |
| | | | | | | | protein - upland cotton |
| | | | | | | | >gi\|439654\|emb\|CAA53567.1\| (X75954) RS16 |
| | | | | | | | protein, 40S subunit [Gossypium hirsutum] |
| 1042 | LIB3602-035-Q1-K1-B1 | 1173209 | BLASTX | 530 | 8.00E−54 | 75 | 40S RIBOSOMAL PROTEIN S16 |
| | | | | | | | >gi\|541835\|pir\|\|S41193 ribosomal protein S16 |
| | | | | | | | protein - upland cotton |
| | | | | | | | >gi\|439654\|emb\|CAA53567.1\| (X75954) RS16 |
| | | | | | | | protein, 40S subunit [Gossypium hirsutum] |
| 1043 | LIB3602-063-Q1-K6-C12 | 1173209 | BLASTX | 530 | 8.00E−54 | 75 | 40S RIBOSOMAL PROTEIN S16 |
| | | | | | | | >gi\|541835\|pir\|\|S41193 ribosomal protein S16 |
| | | | | | | | protein - upland cotton |
| | | | | | | | >gi\|439654\|emb\|CAA53567.1\| (X75954) RS16 |
| | | | | | | | protein, 40S subunit [Gossypium hirsutum] |
| 1044 | LIB3602-101-Q1-K1-H1 | 1173209 | BLASTX | 429 | 5.00E−53 | 82 | 40S RIBOSOMAL PROTEIN S16 |
| | | | | | | | >gi\|541835\|pir\|\|S41193 ribosomal protein S16 |
| | | | | | | | protein - upland cotton |
| | | | | | | | >gi\|439654\|emb\|CAA53567.1\| (X75954) RS16 |
| | | | | | | | protein, 40S subunit [Gossypium hirsutum] |
| 1045 | LIB3602-022-Q6-K1-H6 | 1173209 | BLASTX | 515 | 2.00E−52 | 77 | 40S RIBOSOMAL PROTEIN S16 |
| | | | | | | | >gi\|541835\|pir\|\|S41193 ribosomal protein S16 |
| | | | | | | | protein - upland cotton |
| | | | | | | | >gi\|439654\|emb\|CAA53567.1\| (X75954) RS16 |
| | | | | | | | protein, 40S subunit [Gossypium hirsutum] |
| 1046 | LIB3602-107-Q1-K1-D4 | 1173209 | BLASTX | 275 | 9.00E−50 | 66 | 40S RIBOSOMAL PROTEIN S16 |
| | | | | | | | >gi\|541835\|pir\|\|S41193 ribosomal protein S16 |
| | | | | | | | protein - upland cotton |
| | | | | | | | >gi\|439654\|emb\|CAA53567.1\| (X75954) RS16 |
| | | | | | | | protein, 40S subunit [Gossypium hirsutum] |
| 1047 | LIB3602-066-Q1-K6-D10 | 1173209 | BLASTX | 281 | 7.00E−25 | 65 | 40S RIBOSOMAL PROTEIN S16 |
| | | | | | | | >gi\|541835\|pir\|\|S41193 ribosomal protein S16 |
| | | | | | | | protein - upland cotton |
| | | | | | | | >gi\|439654\|emb\|CAA53567.1\| (X75954) RS16 |
| | | | | | | | protein, 40S subunit [Gossypium hirsutum] |
| 1048 | LIB3602-093-Q6-K6-C12 | 5932542 | BLASTX | 473 | 2.00E−47 | 77 | (AC009465) putative ribosomal protein s19 or s24 |
| | | | | | | | [Arabidopsis thaliana] |
| 1049 | LIB3602-012-Q6-K1-G9 | 5932542 | BLASTX | 469 | 7.00E−47 | 78 | (AC009465) putative ribosomal protein s19 or s24 |
| | | | | | | | [Arabidopsis thaliana] |
| 1050 | LIB3602-045-Q6-K1-H11 | 5932542 | BLASTX | 469 | 7.00E−47 | 78 | (AC009465) putative ribosomal protein s19 or s24 |
| | | | | | | | [Arabidopsis thaliana] |
| 1051 | LIB3602-060-Q6-K1-A8 | 5932542 | BLASTX | 466 | 2.00E−46 | 77 | (AC009465) putative ribosomal protein s19 or s24 |
| | | | | | | | [Arabidopsis thaliana] |
| 1052 | LIB3602-087-Q6-K1-F5 | 5932542 | BLASTX | 441 | 1.00E−43 | 76 | (AC009465) putative ribosomal protein s19 or s24 |
| | | | | | | | [Arabidopsis thaliana] |
| 1053 | LIB3602-025-Q6-K1-D8 | 5932542 | BLASTX | 341 | 4.00E−32 | 75 | (AC009465) putative ribosomal protein s19 or s24 |
| | | | | | | | [Arabidopsis thaliana] |
| 1054 | LIB3602-062-Q6-K1-H12 | 5932542 | BLASTX | 333 | 4.00E−31 | 74 | (AC009465) putative ribosomal protein s19 or s24 |
| | | | | | | | [Arabidopsis thaliana] |
| 1055 | LIB3602-119-Q1-K1-C12 | 5932542 | BLASTX | 290 | 2.00E−26 | 80 | (AC009465) putative ribosomal protein s19 or s24 |
| | | | | | | | [Arabidopsis thaliana] |
| 1056 | LIB3602-107-Q1-K1-F9 | 7428635 | BLASTX | 261 | 2.00E−22 | 42 | cyanoglobin - Synechocystis sp. (strain PCC 6803) |
| | | | | | | | >gi\|1653074\|dbj\|BAA17991.1\| (D90910) |
| | | | | | | | cyanoglobin [Synechocystis sp.] |
| 1057 | LIB3602-001-P1-K6-A10 | 7428635 | BLASTX | 261 | 2.00E−22 | 42 | cyanoglobin - Synechocystis sp. (strain PCC 6803) |
| | | | | | | | >gi\|1653074\|dbj\|BAA17991.1\| (D90910) |
| | | | | | | | cyanoglobin [Synechocystis sp.] |
| 1058 | LIB3602-089-Q6-K6-E2 | 7428635 | BLASTX | 259 | 4.00E−22 | 45 | cyanoglobin - Synechocystis sp. (strain PCC 6803) |
| | | | | | | | >gi\|1653074\|dbj\|BAA17991.1\| (D90910) |
| | | | | | | | cyanoglobin [Synechocystis sp.] |
| 1059 | LIB3602-056-Q6-K1-A1 | 7428635 | BLASTX | 194 | 1.00E−14 | 42 | cyanoglobin - Synechocystis sp. (strain PCC 6803) |
| | | | | | | | >gi\|1653074\|dbj\|BAA17991.1\| (D90910) |
| | | | | | | | cyanoglobin [Synechocystis sp.] |
| 1060 | LIB3602-023-Q6-K1-B2 | 7428635 | BLASTX | 183 | 3.00E−13 | 41 | cyanoglobin - Synechocystis sp. (strain PCC 6803) |
| | | | | | | | >gi\|1653074\|dbj\|BAA17991.1\| (D90910) |
| | | | | | | | cyanoglobin [Synechocystis sp.] |
| 1061 | LIB3602-050-Q6-K1-F8 | 7428635 | BLASTX | 171 | 6.00E−12 | 45 | cyanoglobin - Synechocystis sp. (strain PCC 6803) |
| | | | | | | | >gi\|1653074\|dbj\|BAA17991.1\| (D90910) |
| | | | | | | | cyanoglobin [Synechocystis sp.] |
| 1062 | LIB3602-100-Q1-K1-G12 | 7428635 | BLASTX | 166 | 2.00E−11 | 42 | cyanoglobin - Synechocystis sp. (strain PCC 6803) |
| | | | | | | | >gi\|1653074\|dbj\|BAA17991.1\| (D90910) |
| | | | | | | | cyanoglobin [Synechocystis sp.] |
| 1063 | LIB3602-053-Q6-K1-H5 | 7428635 | BLASTX | 151 | 1.00E−09 | 43 | cyanoglobin - Synechocystis sp. (strain PCC 6803) |
| | | | | | | | >gi\|1653074\|dbj\|BAA17991.1\| (D90910) |
| | | | | | | | cyanoglobin [Synechocystis sp.] |
| 1064 | LIB3602-078-Q6-K6-G8 | 2407314 | BLASTX | 691 | 9.00E−73 | 74 | (AF017998) chlorophyll a/b binding protein |
| | | | | | | | [Tetraselmis sp. RG-15] |
| 1065 | LIB3602-088-Q6-K6-H5 | 2407314 | BLASTX | 524 | 2.00E−67 | 70 | (AF017998) chlorophyll a/b binding protein |
| | | | | | | | [Tetraselmis sp. RG-15] |
| 1066 | LIB3602-023-Q6-K1-D5 | 2407314 | BLASTX | 591 | 4.00E−61 | 72 | (AF017998) chlorophyll a/b binding protein |
| | | | | | | | [Tetraselmis sp. RG-15] |
| 1067 | LIB3602-049-Q6-K1-F1 | 2407314 | BLASTX | 544 | 1.00E−55 | 76 | (AF017998) chlorophyll a/b binding protein |
| | | | | | | | [Tetraselmis sp. RG-15] |
| 1068 | LIB3602-032-Q6-K1-H5 | 2407314 | BLASTX | 539 | 4.00E−55 | 76 | (AF017998) chlorophyll a/b binding protein |
| | | | | | | | [Tetraselmis sp. RG-15] |
| 1069 | LIB3602-056-Q6-K1-G1 | 2407314 | BLASTX | 524 | 2.00E−53 | 76 | (AF017998) chlorophyll a/b binding protein |
| | | | | | | | [Tetraselmis sp. RG-15] |
| 1070 | LIB3602-052-Q6-K1-G5 | 2407314 | BLASTX | 479 | 4.00E−48 | 78 | (AF017998) chlorophyll a/b binding protein |
| | | | | | | | [Tetraselmis sp. RG-15] |
| 1071 | LIB3602-043-Q6-K1-A1 | 2407314 | BLASTX | 349 | 5.00E−33 | 75 | (AF017998) chlorophyll a/b binding protein |
| | | | | | | | [Tetraselmis sp. RG-15] |
| 1072 | LIB3602-092-Q6-K6-F7 | 131389 | BLASTX | 724 | 1.00E−76 | 69 | OXYGEN-EVOLVING ENHANCER PROTEIN 2 |
| | | | | | | | PRECURSOR (OEE2) >gi\|81253\|pir\|\|S00413 |
| | | | | | | | photosystem II oxygen-evolving complex protein 2 |
| | | | | | | | precursor - Chlamydomonas reinhardtii |
| | | | | | | | >gi\|167428\|gb\|AAA33088.1\| (M15187) oxygen- |
| | | | | | | | evolving enhancer protein 2 [Chlamydomonas |
| | | | | | | | reinhardt |
| 1073 | LIB3602-015-Q6-K1-H5 | 131389 | BLASTX | 694 | 4.00E−73 | 69 | OXYGEN-EVOLVING ENHANCER PROTEIN 2 |
| | | | | | | | PRECURSOR (OEE2) >gi\|81253\|pir\|\|S00413 |
| | | | | | | | photosystem II oxygen-evolving complex protein 2 |
| | | | | | | | precursor - Chlamydomonas reinhardtii |
| | | | | | | | >gi\|167428\|gb\|AAA33088.1\| (M15187) oxygen- |
| | | | | | | | evolving enhancer protein 2 [Chlamydomonas |
| | | | | | | | reinhardt |
| 1074 | LIB3602-081-Q6-K6-D8 | 131389 | BLASTX | 568 | 2.00E−58 | 70 | OXYGEN-EVOLVING ENHANCER PROTEIN 2 |
| | | | | | | | PRECURSOR (OEE2) >gi\|81253\|pir\|\|S00413 |
| | | | | | | | photosystem II oxygen-evolving complex protein 2 |
| | | | | | | | precursor - Chlamydomonas reinhardtii |
| | | | | | | | >gi\|167428\|gb\|AAA33088.1\| (M15187) oxygen- |
| | | | | | | | evolving enhancer protein 2 [Chlamydomonas |
| | | | | | | | reinhardt |
| 1075 | LIB3602-009-Q6-K1-H12 | 131389 | BLASTX | 391 | 8.00E−38 | 57 | OXYGEN-EVOLVING ENHANCER PROTEIN 2 |
| | | | | | | | PRECURSOR (OEE2) >gi\|81253\|pir\|\|S00413 |
| | | | | | | | photosystem II oxygen-evolving complex protein 2 |
| | | | | | | | precursor - Chlamydomonas reinhardtii |
| | | | | | | | >gi\|167428\|gb\|AAA33088.1\| (M15187) oxygen- |
| | | | | | | | evolving enhancer protein 2 [Chlamydomonas |
| | | | | | | | reinhardt |
| 1076 | LIB3602-017-Q6-K1-C9 | 131389 | BLASTX | 374 | 1.00E−35 | 77 | OXYGEN-EVOLVING ENHANCER PROTEIN 2 |
| | | | | | | | PRECURSOR (OEE2) >gi\|81253\|pir\|\|S00413 |
| | | | | | | | photosystem II oxygen-evolving complex protein 2 |
| | | | | | | | precursor - Chlamydomonas reinhardtii |
| | | | | | | | >gi\|167428\|gb\|AAA33088.1\| (M15187) oxygen- |
| | | | | | | | evolving enhancer protein 2 [Chlamydomonas |
| | | | | | | | reinhardt |
| 1077 | LIB3602-047-Q6-K1-E7 | 131389 | BLASTX | 356 | 1.00E−33 | 58 | OXYGEN-EVOLVING ENHANCER PROTEIN 2 |
| | | | | | | | PRECURSOR (OEE2) >gi\|81253\|pir\|\|S00413 |
| | | | | | | | photosystem II oxygen-evolving complex protein 2 |
| | | | | | | | precursor - Chlamydomonas reinhardtii |
| | | | | | | | >gi\|167428\|gb\|AAA33088.1\| (M15187) oxygen- |
| | | | | | | | evolving enhancer protein 2 [Chlamydomonas |
| | | | | | | | reinhardt |
| 1078 | LIB3602-040-Q6-K1-D4 | 131389 | BLASTX | 323 | 9.00E−30 | 59 | OXYGEN-EVOLVING ENHANCER PROTEIN 2 |
| | | | | | | | PRECURSOR (OEE2) >gi\|81253\|pir\|\|S00413 |
| | | | | | | | photosystem II oxygen-evolving complex protein 2 |
| | | | | | | | precursor - Chlamydomonas reinhardtii |
| | | | | | | | >gi\|167428\|gb\|AAA33088.1\| (M15187) oxygen- |
| | | | | | | | evolving enhancer protein 2 [Chlamydomonas |
| | | | | | | | reinhardt |
| 1079 | LIB3602-037-Q6-K1-C4 | 131389 | BLASTX | 277 | 2.00E−24 | 80 | OXYGEN-EVOLVING ENHANCER PROTEIN 2 |
| | | | | | | | PRECURSOR (OEE2) >gi\|81253\|pir\|\|S00413 |
| | | | | | | | photosystem II oxygen-evolving complex protein 2 |
| | | | | | | | precursor - Chlamydomonas reinhardtii |
| | | | | | | | >gi\|167428\|gb\|AAA33088.1\| (M15187) oxygen- |
| | | | | | | | evolving enhancer protein 2 [Chlamydomonas |
| | | | | | | | reinhardt |
| 1080 | LIB3602-104-Q1-K1-B9 | 6723436 | BLASTX | 439 | 3.00E−43 | 64 | (AL132965) 60S RIBOSOMAL PROTEIN-like |
| | | | | | | | [Arabidopsis thaliana] |
| 1081 | LIB3602-088-Q6-K6-D6 | 6723436 | BLASTX | 439 | 3.00E−43 | 64 | (AL132965) 60S RIBOSOMAL PROTEIN-like |
| | | | | | | | [Arabidopsis thaliana] |
| 1082 | LIB3602-048-Q6-K1QA-C7 | 6723436 | BLASTX | 439 | 3.00E−43 | 64 | (AL132965) 60S RIBOSOMAL PROTEIN-like |
| | | | | | | | [Arabidopsis thaliana] |
| 1083 | LIB3602-011-Q6-K1-E12 | 6723436 | BLASTX | 431 | 2.00E−42 | 63 | (AL132965) 60S RIBOSOMAL PROTEIN-like |
| | | | | | | | [Arabidopsis thaliana] |
| 1084 | LIB3602-017-Q6-K1-A2 | 6723436 | BLASTX | 268 | 2.00E−23 | 59 | (AL132965) 60S RIBOSOMAL PROTEIN-like |
| | | | | | | | [Arabidopsis thaliana] |
| 1085 | LIB3602-068-Q1-K1-D7 | 6723436 | BLASTX | 222 | 9.00E−18 | 89 | (AL132965) 60S RIBOSOMAL PROTEIN-like |
| | | | | | | | [Arabidopsis thaliana] |
| 1086 | LIB3602-078-Q6-K6-B9 | 6723436 | BLASTX | 197 | 3.00E−15 | 54 | (AL132965) 60S RIBOSOMAL PROTEIN-like |
| | | | | | | | [Arabidopsis thaliana] |
| 1087 | LIB3602-066-Q1-K6-B9 | 6723436 | BLASTX | 173 | 6.00E−13 | 88 | (AL132965) 60S RIBOSOMAL PROTEIN-like |
| | | | | | | | [Arabidopsis thaliana] |
| 1088 | LIB3602-029-Q6-K1-E7 | 7488205 | BLASTX | 205 | 2.00E−20 | 43 | probable serine/threonine-specific protein kinase |
| | | | | | | | (EC 2.7.1.—) T22A6.310 - Arabidopsis thaliana |
| | | | | | | | >gi\|5051790\|emb\|CAB45083.1\| (AL078637) |
| | | | | | | | putative protein kinase [Arabidopsis thaliana] |
| | | | | | | | >gi\|7269298\|emb\|CAB79358.1\| (AL161561) |
| | | | | | | | putative protein kinase [Arabidops |
| 1089 | LIB3602-034-Q6-K1-B4 | 7488205 | BLASTX | 187 | 8.00E−14 | 40 | probable serine/threonine-specific protein kinase |
| | | | | | | | (EC 2.7.1.—) T22A6.310 - Arabidopsis thaliana |
| | | | | | | | >gi\|5051790\|emb\|CAB45083.1\| (AL078637) |
| | | | | | | | putative protein kinase [Arabidopsis thaliana] |
| | | | | | | | >gi\|7269298\|emb\|CAB79358.1\| (AL161561) |
| | | | | | | | putative protein kinase [Arabidops |
| 1090 | LIB3602-010-Q6-K1-H10 | 7488205 | BLASTX | 187 | 1.00E−13 | 38 | probable serine/threonine-specific protein kinase |
| | | | | | | | (EC 2.7.1.—) T22A6.310 - Arabidopsis thaliana |
| | | | | | | | >gi\|5051790\|emb\|CAB45083.1\| (AL078637) |
| | | | | | | | putative protein kinase [Arabidopsis thaliana] |
| | | | | | | | >gi\|7269298\|emb\|CAB79358.1\| (AL161561) |
| | | | | | | | putative protein kinase [Arabidops |
| 1091 | LIB3602-035-Q1-K1-B4 | 7488205 | BLASTX | 181 | 5.00E−13 | 40 | probable serine/threonine-specific protein kinase |
| | | | | | | | (EC 2.7.1.—) T22A6.310 - Arabidopsis thaliana |
| | | | | | | | >gi\|5051790\|emb\|CAB45083.1\| (AL078637) |
| | | | | | | | putative protein kinase [Arabidopsis thaliana] |
| | | | | | | | >gi\|7269298\|emb\|CAB79358.1\| (AL161561) |
| | | | | | | | putative protein kinase [Arabidops |
| 1092 | LIB3602-019-Q6-K1-C7 | 7488205 | BLASTX | 181 | 5.00E−13 | 28 | probable serine/threonine-specific protein kinase |
| | | | | | | | (EC 2.7.1.—) T22A6.310 - Arabidopsis thaliana |
| | | | | | | | >gi\|5051790\|emb\|CAB45083.1\| (AL078637) |
| | | | | | | | putative protein kinase [Arabidopsis thaliana] |
| | | | | | | | >gi\|7269298\|emb\|CAB79358.1\| (AL161561) |
| | | | | | | | putative protein kinase [Arabidops |
| 1093 | LIB3602-073-Q1-K1-B12 | 7488205 | BLASTX | 180 | 6.00E−13 | 39 | probable serine/threonine-specific protein kinase |
| | | | | | | | (EC 2.7.1.—) T22A6.310 - Arabidopsis thaliana |
| | | | | | | | >gi\|5051790\|emb\|CAB45083.1\| (AL078637) |
| | | | | | | | putative protein kinase [Arabidopsis thaliana] |
| | | | | | | | >gi\|7269298\|emb\|CAB79358.1\| (AL161561) |
| | | | | | | | putative protein kinase [Arabidops |
| 1094 | LIB3602-025-Q6-K1-G5 | 7488205 | BLASTX | 171 | 7.00E−12 | 31 | probable serine/threonine-specific protein kinase |
| | | | | | | | (EC 2.7.1.—) T22A6.310 - Arabidopsis thaliana |
| | | | | | | | >gi\|5051790\|emb\|CAB45083.1\| (AL078637) |
| | | | | | | | putative protein kinase [Arabidopsis thaliana] |
| | | | | | | | >gi\|7269298\|emb\|CAB79358.1\| (AL161561) |
| | | | | | | | putative protein kinase [Arabidops |
| 1095 | LIB3602-058-Q6-K1-A7 | 7488205 | BLASTX | 150 | 2.00E−09 | 35 | probable serine/threonine-specific protein kinase |
| | | | | | | | (EC 2.7.1.—) T22A6.310 - Arabidopsis thaliana |
| | | | | | | | >gi\|5051790\|emb\|CAB45083.1\| (AL078637) |
| | | | | | | | putative protein kinase [Arabidopsis thaliana] |
| | | | | | | | >gi\|7269298\|emb\|CAB79358.1\| (AL161561) |
| | | | | | | | putative protein kinase [Arabidops |
| 1096 | LIB3602-023-Q6-K1-B3 | 7630007 | BLASTX | 783 | 1.00E−83 | 89 | (AL132960) ribosomal protein S3a homolog |
| | | | | | | | [Arabidopsis thaliana] |
| 1097 | LIB3602-115-Q1-K1-B10 | 7630007 | BLASTX | 737 | 3.00E−78 | 89 | (AL132960) ribosomal protein S3a homolog |
| | | | | | | | [Arabidopsis thaliana] |
| 1098 | LIB3602-018-Q6-K1-G12 | 7630007 | BLASTX | 722 | 2.00E−76 | 88 | (AL132960) ribosomal protein S3a homolog |
| | | | | | | | [Arabidopsis thaliana] |
| 1099 | LIB3602-058-Q6-K1-F9 | 7630007 | BLASTX | 708 | 7.00E−75 | 89 | (AL132960) ribosomal protein S3a homolog |
| | | | | | | | [Arabidopsis thaliana] |
| 1100 | LIB3602-010-Q6-K1-C7 | 7630007 | BLASTX | 695 | 3.00E−73 | 89 | (AL132960) ribosomal protein S3a homolog |
| | | | | | | | [Arabidopsis thaliana] |
| 1101 | LIB3602-047-Q6-K1-D9 | 7630007 | BLASTX | 665 | 8.00E−70 | 89 | (AL132960) ribosomal protein S3a homolog |
| | | | | | | | [Arabidopsis thaliana] |
| 1102 | LIB3602-065-Q1-K6-A10 | 7630007 | BLASTX | 664 | 1.00E−69 | 91 | (AL132960) ribosomal protein S3a homolog |
| | | | | | | | [Arabidopsis thaliana] |
| 1103 | LIB3602-065-Q1-K6-H1 | 7630007 | BLASTX | 632 | 6.00E−66 | 86 | (AL132960) ribosomal protein S3a homolog |
| | | | | | | | [Arabidopsis thaliana] |
| 1104 | LIB3602-012-Q6-K1-C6 | 6729494 | BLASTX | 680 | 2.00E−71 | 78 | (AL132966) 60S RIBOSOMAL PROTEIN L12- |
| | | | | | | | like [Arabidopsis thaliana] |
| 1105 | LIB3602-057-Q6-K1-B6 | 6729494 | BLASTX | 680 | 2.00E−71 | 78 | (AL132966) 60S RIBOSOMAL PROTEIN L12- |
| | | | | | | | like [Arabidopsis thaliana] |
| 1106 | LIB3602-072-Q1-K1-H8 | 6729494 | BLASTX | 667 | 6.00E−70 | 78 | (AL132966) 60S RIBOSOMAL PROTEIN L12- |
| | | | | | | | like [Arabidopsis thaliana] |
| 1107 | LIB3602-015-Q6-K1-B5 | 6729494 | BLASTX | 448 | 2.00E−44 | 75 | (AL132966) 60S RIBOSOMAL PROTEIN L12- |
| | | | | | | | like [Arabidopsis thaliana] |
| 1108 | LIB3602-020-Q6-K1-A4 | 6729494 | BLASTX | 399 | 9.00E−39 | 74 | (AL132966) 60S RIBOSOMAL PROTEIN L12- |
| | | | | | | | like [Arabidopsis thaliana] |
| 1109 | LIB3602-059-Q6-K1-A1 | 6729494 | BLASTX | 285 | 1.00E−25 | 65 | (AL132966) 60S RIBOSOMAL PROTEIN L12- |
| | | | | | | | like [Arabidopsis thaliana] |
| 1110 | LIB3602-043-Q6-K1-C5 | 6729494 | BLASTX | 176 | 4.00E−13 | 64 | (AL132966) 60S RIBOSOMAL PROTEIN L12- |
| | | | | | | | like [Arabidopsis thaliana] |
| 1111 | LIB3602-078-Q6-K6-F12 | 5762260 | BLASTX | 693 | 5.00E−73 | 75 | (AB008848) Csf-3 [Cucumis sativus] |
| 1112 | LIB3602-112-Q1-K1-C2 | 5762260 | BLASTX | 655 | 1.00E−68 | 74 | (AB008848) Csf-3 [Cucumis sativus] |
| 1113 | LIB3602-093-Q6-K6-E1 | 5762260 | BLASTX | 266 | 5.00E−45 | 65 | (AB008848) Csf-3 [Cucumis sativus] |
| 1114 | LIB3602-086-Q6-K1-H5 | 5762260 | BLASTX | 312 | 9.00E−43 | 74 | (AB008848) Csf-3 [Cucumis sativus] |
| 1115 | LIB3602-026-Q6-K1-C10 | 5762260 | BLASTX | 406 | 2.00E−39 | 72 | (AB008848) Csf-3 [Cucumis sativus] |
| 1116 | LIB3602-088-Q6-K6-B11 | 5762260 | BLASTX | 312 | 1.00E−31 | 74 | (AB008848) Csf-3 [Cucumis sativus] |
| 1117 | LIB3602-118-Q1-K1-B11 | 5762260 | BLASTX | 206 | 7.00E−30 | 70 | (AB008848) Csf-3 [Cucumis sativus] |
| 1118 | LIB3602-116-Q1-K1-G11 | 1702984 | BLASTX | 762 | 1.00E−100 | 90 | 14-3-3-LIKE PROTEIN >gi\|1361946\|pir\|\|S57283 |
| | | | | | | | 14-3-3 brain protein homolog - Chlamydomonas |
| | | | | | | | reinhardtii >gi\|1015462\|emb\|CAA55964.1\| |
| | | | | | | | (X79445) 14-3-3 protein [Chlamydomonas |
| | | | | | | | reinhardtii] |
| 1119 | LIB3602-094-Q6-K6-G11 | 1702984 | BLASTX | 879 | 2.00E−94 | 92 | 14-3-3-LIKE PROTEIN >gi\|1361946\|pir\|\|S57283 |
| | | | | | | | 14-3-3 brain protein homolog - Chlamydomonas |
| | | | | | | | reinhardtii >gi\|1015462\|emb\|CAA55964.1\| |
| | | | | | | | (X79445) 14-3-3 protein [Chlamydomonas |
| | | | | | | | reinhardtii] |
| 1120 | LIB3602-014-Q6-K1-C8 | 1702984 | BLASTX | 725 | 8.00E−77 | 96 | 14-3-3-LIKE PROTEIN >gi\|1361946\|pir\|\|S57283 |
| | | | | | | | 14-3-3 brain protein homolog - Chlamydomonas |
| | | | | | | | reinhardtii >gi\|1015462\|emb\|CAA55964.1\| |
| | | | | | | | (X79445) 14-3-3 protein [Chlamydomonas |
| | | | | | | | reinhardtii] |
| 1121 | LIB3602-050-Q6-K1-C10 | 1702984 | BLASTX | 331 | 1.00E−30 | 96 | 14-3-3-LIKE PROTEIN >gi\|1361946\|pir\|\|S57283 |
| | | | | | | | 14-3-3 brain protein homolog - Chlamydomonas |
| | | | | | | | reinhardtii >gi\|1015462\|emb\|CAA55964.1\| |
| | | | | | | | (X79445) 14-3-3 protein [Chlamydomonas |
| | | | | | | | reinhardtii] |
| 1122 | LIB3602-016-Q6-K1-A9 | 1702984 | BLASTX | 291 | 5.00E−26 | 98 | 14-3-3-LIKE PROTEIN >gi\|1361946\|pir\|\|S57283 |
| | | | | | | | 14-3-3 brain protein homolog - Chlamydomonas |
| | | | | | | | reinhardtii >gi\|1015462\|emb\|CAA55964.1\| |
| | | | | | | | (X79445) 14-3-3 protein [Chlamydomonas |
| | | | | | | | reinhardtii] |
| 1123 | LIB3602-026-Q6-K1-F8 | 1702984 | BLASTX | 291 | 6.00E−26 | 98 | 14-3-3-LIKE PROTEIN >gi\|1361946\|pir\|\|S57283 |
| | | | | | | | 14-3-3 brain protein homolog - Chlamydomonas |
| | | | | | | | reinhardtii >gi\|1015462\|emb\|CAA55964.1\| |
| | | | | | | | (X79445) 14-3-3 protein [Chlamydomonas |
| | | | | | | | reinhardtii] |
| 1124 | LIB3602-048-Q6-K1QA- | 1702984 | BLASTX | 277 | 3.00E−24 | 98 | 14-3-3-LIKE PROTEIN >gi\|1361946\|pir\|\|S57283 |
| | G12 | | | | | | 14-3-3 brain protein homolog - Chlamydomonas |
| | | | | | | | reinhardtii >gi\|1015462\|emb\|CAA55964.1\| |
| | | | | | | | (X79445) 14-3-3 protein [Chlamydomonas |
| | | | | | | | reinhardtii] |
| 1125 | LIB3602-015-Q6-K1-H7 | 1710565 | BLASTX | 428 | 5.00E−42 | 81 | 60S RIBOSOMAL PROTEIN L44 (L41) |
| | | | | | | | >gi\|1255906\|dbj\|BAA11057.1\| (D67040) ribosomal |
| | | | | | | | protein L41 [Candida utilis] |
| 1126 | LIB3602-047-Q6-K1-G7 | 1710565 | BLASTX | 377 | 4.00E−36 | 83 | 60S RIBOSOMAL PROTEIN L44 (L41) |
| | | | | | | | >gi\|1255906\|dbj\|BAA11057.1\| (D67040) ribosomal |
| | | | | | | | protein L41 [Candida utilis] |
| 1127 | LIB3602-090-Q6-K6-A9 | 1710565 | BLASTX | 376 | 7.00E−36 | 79 | 60S RIBOSOMAL PROTEIN L44 (L41) |
| | | | | | | | >gi\|1255906\|dbj\|BAA11057.1\| (D67040) ribosomal |
| | | | | | | | protein L41 [Candida utilis] |
| 1128 | LIB3602-049-Q6-K1-H6 | 1710565 | BLASTX | 342 | 5.00E−32 | 82 | 60S RIBOSOMAL PROTEIN L44 (L41) |
| | | | | | | | >gi\|1255906\|dbj\|BAA11057.1\| (D67040) ribosomal |
| | | | | | | | protein L41 [Candida utilis] |
| 1129 | LIB3602-082-Q6-K6-A2 | 1710565 | BLASTX | 234 | 1.00E−28 | 74 | 60S RIBOSOMAL PROTEIN L44 (L41) |
| | | | | | | | >gi\|1255906\|dbj\|BAA11057.1\| (D67040) ribosomal |
| | | | | | | | protein L41 [Candida utilis] |
| 1130 | LIB3602-048-Q6-K1QA-E5 | 1710565 | BLASTX | 282 | 6.00E−25 | 62 | 60S RIBOSOMAL PROTEIN L44 (L41) |
| | | | | | | | >gi\|1255906\|dbj\|BAA11057.1\| (D67040) ribosomal |
| | | | | | | | protein L41 [Candida utilis] |
| 1131 | LIB3602-088-Q6-K6-F8 | 1710565 | BLASTX | 275 | 5.00E−24 | 80 | 60S RIBOSOMAL PROTEIN L44 (L41) |
| | | | | | | | >gi\|1255906\|dbj\|BAA11057.1\| (D67040) ribosomal |
| | | | | | | | protein L41 [Candida utilis] |
| 1132 | LIB3602-107-Q1-K1-C8 | 119167 | BLASTX | 415 | 1.00E−40 | 83 | ELONGATION FACTOR 2 (EF-2) |
| | | | | | | | >gi\|421771\|pir\|\|S32819 translation elongation factor |
| | | | | | | | eEF-2 - Chlorella kessleri |
| | | | | | | | >gi\|167245\|gb\|AAA33028.1\| (M68064) elongation |
| | | | | | | | factor 2 [Chlorella kessleri] |
| | | | | | | | >gi\|228693\|prf\|\|1808323A elongation factor 2 |
| | | | | | | | [Chlorella kessleri] |
| 1133 | LIB3602-021-Q6-K1-D11 | 119167 | BLASTX | 400 | 3.00E−39 | 91 | ELONGATION FACTOR 2 (EF-2) |
| | | | | | | | >gi\|421771\|pir\|\|S32819 translation elongation factor |
| | | | | | | | eEF-2 - Chlorella kessleri |
| | | | | | | | >gi\|167245\|gb\|AAA33028.1\| (M68064) elongation |
| | | | | | | | factor 2 [Chlorella kessleri] |
| | | | | | | | >gi\|228693\|prf\|\|1808323A elongation factor 2 |
| | | | | | | | [Chlorella kessleri] |
| 1134 | LIB3602-086-Q6-K1-C8 | 119167 | BLASTX | 372 | 2.00E−35 | 58 | ELONGATION FACTOR 2 (EF-2) |
| | | | | | | | >gi\|421771\|pir\|\|S32819 translation elongation factor |
| | | | | | | | eEF-2 - Chlorella kessleri |
| | | | | | | | >gi\|167245\|gb\|AAA33028.1\| (M68064) elongation |
| | | | | | | | factor 2 [Chlorella kessleri] |
| | | | | | | | >gi\|228693\|prf\|\|1808323A elongation factor 2 |
| | | | | | | | [Chlorella kessleri] |
| 1135 | LIB3602-071-Q1-K1-A12 | 119167 | BLASTX | 338 | 1.00E−31 | 79 | ELONGATION FACTOR 2 (EF-2) |
| | | | | | | | >gi\|421771\|pir\|\|S32819 translation elongation factor |
| | | | | | | | eEF-2 - Chlorella kessleri |
| | | | | | | | >gi\|167245\|gb\|AAA33028.1\| (M68064) elongation |
| | | | | | | | factor 2 [Chlorella kessleri] |
| | | | | | | | >gi\|228693\|prf\|\|1808323A elongation factor 2 |
| | | | | | | | [Chlorella kessleri] |
| 1136 | LIB3602-066-Q1-K6-A12 | 119167 | BLASTX | 318 | 3.00E−29 | 76 | ELONGATION FACTOR 2 (EF-2) |
| | | | | | | | >gi\|421771\|pir\|\|S32819 translation elongation factor |
| | | | | | | | eEF-2 - Chlorella kessleri |
| | | | | | | | >gi\|167245\|gb\|AAA33028.1\| (M68064) elongation |
| | | | | | | | factor 2 [Chlorella kessleri] |
| | | | | | | | >gi\|228693\|prf\|\|1808323A elongation factor 2 |
| | | | | | | | [Chlorella kessleri] |
| 1137 | LIB3602-014-Q6-K1-C3 | 119167 | BLASTX | 215 | 2.00E−17 | 78 | ELONGATION FACTOR 2 (EF-2) |
| | | | | | | | >gi\|421771\|pir\|\|S32819 translation elongation factor |
| | | | | | | | eEF-2 - Chlorella kessleri |
| | | | | | | | >gi\|167245\|gb\|AAA33028.1\| (M68064) elongation |
| | | | | | | | factor 2 [Chlorella kessleri] |
| | | | | | | | >gi\|228693\|prf\|\|1808323A elongation factor 2 |
| | | | | | | | [Chlorella kessleri] |
| 1138 | LIB3602-014-Q6-K1-E6 | 119167 | BLASTX | 170 | 1.00E−11 | 41 | ELONGATION FACTOR 2 (EF-2) |
| | | | | | | | >gi\|421771\|pir\|\|S32819 translation elongation factor |
| | | | | | | | eEF-2 - Chlorella kessleri |
| | | | | | | | >gi\|167245\|gb\|AAA33028.1\| (M68064) elongation |
| | | | | | | | factor 2 [Chlorella kessleri] |
| | | | | | | | >gi\|228693\|prf\|\|1808323A elongation factor 2 |
| | | | | | | | [Chlorella kessleri] |
| 1139 | LIB3602-076-Q6-K6-E12 | 6225750 | BLASTX | 524 | 3.00E−53 | 68 | NUCLEOSIDE DIPHOSPHATE KINASE I (NDK |
| | | | | | | | I) (NDP KINASE I) (NDPK I) |
| | | | | | | | >gi\|3309053\|gb\|AAC25999.1\| (AF072289) |
| | | | | | | | nucleoside diphosphate kinase I |
| | | | | | | | [Mesembryanthemum crystallinum] |
| 1140 | LIB3602-092-Q6-K6-D6 | 6225750 | BLASTX | 524 | 3.00E−53 | 68 | NUCLEOSIDE DIPHOSPHATE KINASE I (NDK |
| | | | | | | | I) (NDP KINASE I) (NDPK I) |
| | | | | | | | >gi\|3309053\|gb\|AAC25999.1\| (AF072289) |
| | | | | | | | nucleoside diphosphate kinase I |
| | | | | | | | [Mesembryanthemum crystallinum] |
| 1141 | LIB3602-115-Q1-K1-H3 | 6225750 | BLASTX | 427 | 8.00E−42 | 68 | NUCLEOSIDE DIPHOSPHATE KINASE I (NDK |
| | | | | | | | I) (NDP KINASE I) (NDPK I) |
| | | | | | | | >gi\|3309053\|gb\|AAC25999.1\| (AF072289) |
| | | | | | | | nucleoside diphosphate kinase I |
| | | | | | | | [Mesembryanthemum crystallinum] |
| 1142 | LIB3602-114-Q1-K1-B8 | 6225750 | BLASTX | 378 | 5.00E−36 | 73 | NUCLEOSIDE DIPHOSPHATE KINASE I (NDK |
| | | | | | | | I) (NDP KINASE I) (NDPK I) |
| | | | | | | | >gi\|3309053\|gb\|AAC25999.1\| (AF072289) |
| | | | | | | | nucleoside diphosphate kinase I |
| | | | | | | | [Mesembryanthemum crystallinum] |
| 1143 | LIB3602-105-Q1-K1-F6 | 6225750 | BLASTX | 281 | 1.00E−24 | 69 | NUCLEOSIDE DIPHOSPHATE KINASE I (NDK |
| | | | | | | | I) (NDP KINASE I) (NDPK I) |
| | | | | | | | >gi\|3309053\|gb\|AAC25999.1\| (AF072289) |
| | | | | | | | nucleoside diphosphate kinase I |
| | | | | | | | [Mesembryanthemum crystallinum] |
| 1144 | LIB3602-062-Q6-K1-F1 | 6225750 | BLASTX | 262 | 1.00E−22 | 68 | NUCLEOSIDE DIPHOSPHATE KINASE I (NDK |
| | | | | | | | I) (NDP KINASE I) (NDPK I) |
| | | | | | | | >gi\|3309053\|gb\|AAC25999.1\| (AF072289) |
| | | | | | | | nucleoside diphosphate kinase I |
| | | | | | | | [Mesembryanthemum crystallinum] |
| 1145 | LIB3602-093-Q6-K6-B8 | 6225750 | BLASTX | 152 | 1.00E−09 | 53 | NUCLEOSIDE DIPHOSPHATE KINASE I (NDK |
| | | | | | | | I) (NDP KINASE I) (NDPK I) |
| | | | | | | | >gi\|3309053\|gb\|AAC25999.1\| (AF072289) |
| | | | | | | | nucleoside diphosphate kinase I |
| | | | | | | | [Mesembryanthemum crystallinum] |
| 1146 | LIB3602-062-Q6-K1-E12 | 7461899 | BLASTX | 580 | 8.00E−60 | 62 | translation elongation factor EF-3 homolog A666L - |
| | | | | | | | Chlorella virus PBCV-1 |
| | | | | | | | >gi\|2447123\|gb\|AAC96981.1\| (U42580) Chlorella |
| | | | | | | | virus CVK2 translation elongation factor-3 |
| | | | | | | | homolog, refer to GenBank Accession Number |
| | | | | | | | D16505 [Paramecium bursaria Chlorella virus 1] |
| 1147 | LIB3602-066-Q1-K6-G12 | 7461899 | BLASTX | 511 | 1.00E−51 | 59 | translation elongation factor EF-3 homolog A666L - |
| | | | | | | | Chlorella virus PBCV-1 |
| | | | | | | | >gi\|2447123\|gb\|AAC96981.1\| (U42580) Chlorella |
| | | | | | | | virus CVK2 translation elongation factor-3 |
| | | | | | | | homolog, refer to GenBank Accession Number |
| | | | | | | | D16505 [Paramecium bursaria Chlorella virus 1] |
| 1148 | LIB3602-009-Q6-K1-C12 | 7461899 | BLASTX | 459 | 1.00E−45 | 51 | translation elongation factor EF-3 homolog A666L - |
| | | | | | | | Chlorella virus PBCV-1 |
| | | | | | | | >gi\|2447123\|gb\|AAC96981.1\| (U42580) Chlorella |
| | | | | | | | virus CVK2 translation elongation factor-3 |
| | | | | | | | homolog, refer to GenBank Accession Number |
| | | | | | | | D16505 [Paramecium bursaria Chlorella virus 1] |
| 1149 | LIB3602-045-Q6-K1-A7 | 7461899 | BLASTX | 435 | 7.00E−43 | 61 | translation elongation factor EF-3 homolog A666L - |
| | | | | | | | Chlorella virus PBCV-1 |
| | | | | | | | >gi\|2447123\|gb\|AAC96981.1\| (U42580) Chlorella |
| | | | | | | | virus CVK2 translation elongation factor-3 |
| | | | | | | | homolog, refer to GenBank Accession Number |
| | | | | | | | D16505 [Paramecium bursaria Chlorella virus 1] |
| 1150 | LIB3602-089-Q6-K6-A1 | 7461899 | BLASTX | 336 | 4.00E−38 | 45 | translation elongation factor EF-3 homolog A666L - |
| | | | | | | | Chlorella virus PBCV-1 |
| | | | | | | | >gi\|2447123\|gb\|AAC96981.1\| (U42580) Chlorella |
| | | | | | | | virus CVK2 translation elongation factor-3 |
| | | | | | | | homolog, refer to GenBank Accession Number |
| | | | | | | | D16505 [Paramecium bursaria Chlorella virus 1] |
| 1151 | LIB3602-029-Q6-K1-G9 | 7461899 | BLASTX | 269 | 2.00E−23 | 43 | translation elongation factor EF-3 homolog A666L - |
| | | | | | | | Chlorella virus PBCV-1 |
| | | | | | | | >gi\|2447123\|gb\|AAC96981.1\| (U42580) Chlorella |
| | | | | | | | virus CVK2 translation elongation factor-3 |
| | | | | | | | homolog, refer to GenBank Accession Number |
| | | | | | | | D16505 [Paramecium bursaria Chlorella virus 1] |
| 1152 | LIB3602-059-Q6-K1-G9 | 7461899 | BLASTX | 153 | 7.00E−10 | 33 | translation elongation factor EF-3 homolog A666L - |
| | | | | | | | Chlorella virus PBCV-1 |
| | | | | | | | >gi\|2447123\|gb\|AAC96981.1\| (U42580) Chlorella |
| | | | | | | | virus CVK2 translation elongation factor-3 |
| | | | | | | | homolog, refer to GenBank Accession Number |
| | | | | | | | D16505 [Paramecium bursaria Chlorella virus 1] |
| 1153 | LIB3602-029-Q6-K1-F12 | 7268983 | BLASTX | 638 | 1.00E−66 | 74 | (AL161494) putative ribosomal protein L19 |
| | | | | | | | [Arabidopsis thaliana] |
| 1154 | LIB3602-008-Q6-K1-A5 | 7268983 | BLASTX | 631 | 8.00E−66 | 74 | (AL161494) putative ribosomal protein L19 |
| | | | | | | | [Arabidopsis thaliana] |
| 1155 | LIB3602-070-Q1-K1-C3 | 7268983 | BLASTX | 582 | 5.00E−60 | 72 | (AL161494) putative ribosomal protein L19 |
| | | | | | | | [Arabidopsis thaliana] |
| 1156 | LIB3602-088-Q6-K6-E1 | 7268983 | BLASTX | 516 | 3.00E−52 | 64 | (AL161494) putative ribosomal protein L19 |
| | | | | | | | [Arabidopsis thaliana] |
| 1157 | LIB3602-008-Q6-K1-F11 | 7268983 | BLASTX | 432 | 2.00E−42 | 70 | (AL161494) putative ribosomal protein L19 |
| | | | | | | | [Arabidopsis thaliana] |
| 1158 | LIB3602-083-Q6-K6-E9 | 7268983 | BLASTX | 297 | 1.00E−26 | 66 | (AL161494) putative ribosomal protein L19 |
| | | | | | | | [Arabidopsis thaliana] |
| 1159 | LIB3602-051-Q6-K1-C3 | 7268983 | BLASTX | 175 | 7.00E−14 | 73 | (AL161494) putative ribosomal protein L19 |
| | | | | | | | [Arabidopsis thaliana] |
| 1160 | LIB3602-019-Q6-K1-G12 | 6525246 | BLASTX | 200 | 1.00E−15 | 57 | (U12390) beta-galactosidase alpha peptide [Cloning |
| | | | | | | | vector pSport1] |
| 1161 | LIB3602-040-Q6-K1-F7 | 6525246 | BLASTX | 171 | 4.00E−12 | 52 | (U12390) beta-galactosidase alpha peptide [Cloning |
| | | | | | | | vector pSport1] |
| 1162 | LIB3602-037-Q6-K1-B7 | 6525246 | BLASTX | 166 | 1.00E−11 | 54 | (U12390) beta-galactosidase alpha peptide [Cloning |
| | | | | | | | vector pSport1] |
| 1163 | LIB3602-013-Q6-K1-A12 | 6525246 | BLASTX | 163 | 3.00E−11 | 45 | (U12390) beta-galactosidase alpha peptide [Cloning |
| | | | | | | | vector pSport1] |
| 1164 | LIB3602-118-Q1-K1-B6 | 6525246 | BLASTX | 161 | 6.00E−11 | 46 | (U12390) beta-galactosidase alpha peptide [Cloning |
| | | | | | | | vector pSport1] |
| 1165 | LIB3602-047-Q6-K1-B6 | 6525246 | BLASTX | 158 | 2.00E−10 | 45 | (U12390) beta-galactosidase alpha peptide [Cloning |
| | | | | | | | vector pSport1] |
| 1166 | LIB3602-011-Q6-K1-F12 | 541546 | BLASTX | 850 | 1.00E−91 | 98 | polyubiquitin 5 - Volvox carteri |
| | | | | | | | >gi\|395295\|emb\|CAA52290.1\| (X74214) |
| | | | | | | | polyubiquitin [Volvox carteri] |
| 1167 | LIB3602-045-Q6-K1-E12 | 541546 | BLASTX | 763 | 2.00E−81 | 99 | polyubiquitin 5 - Volvox carteri |
| | | | | | | | >gi\|395295\|emb\|CAA52290.1\| (X74214) |
| | | | | | | | polyubiquitin [Volvox carteri] |
| 1168 | LIB3602-030-Q6-K1-F7 | 541546 | BLASTX | 762 | 3.00E−81 | 100 | polyubiquitin 5 - Volvox carteri |
| | | | | | | | >gi\|395295\|emb\|CAA52290.1\| (X74214) |
| | | | | | | | polyubiquitin [Volvox carteri] |
| 1169 | LIB3602-061-Q6-K1-D11 | 541546 | BLASTX | 670 | 2.00E−70 | 98 | polyubiquitin 5 - Volvox carteri |
| | | | | | | | >gi\|395295\|emb\|CAA52290.1\| (X74214) |
| | | | | | | | polyubiquitin [Volvox carteri] |
| 1170 | LIB3602-030-Q6-K1-D10 | 541546 | BLASTX | 662 | 2.00E−69 | 100 | polyubiquitin 5 - Volvox carteri |
| | | | | | | | >gi\|395295\|emb\|CAA52290.1\| (X74214) |
| | | | | | | | polyubiquitin [Volvox carteri] |
| 1171 | LIB3602-063-Q1-K6-B2 | 541546 | BLASTX | 515 | 3.00E−52 | 99 | polyubiquitin 5 - Volvox carteri |
| | | | | | | | >gi\|395295\|emb\|CAA52290.1\| (X74214) |
| | | | | | | | polyubiquitin [Volvox carteri] |
| 1172 | LIB3602-027-Q6-K1-G7 | 7413634 | BLASTX | 342 | 4.00E−32 | 72 | (AL162971) 60S ribosomal protein-like |
| | | | | | | | [Arabidopsis thaliana] |
| 1173 | LIB3602-022-Q6-K1-B11 | 7413634 | BLASTX | 342 | 5.00E−32 | 72 | (AL162971) 60S ribosomal protein-like |
| | | | | | | | [Arabidopsis thaliana] |
| 1174 | LIB3602-060-Q6-K1-G4 | 7413634 | BLASTX | 342 | 6.00E−32 | 72 | (AL162971) 60S ribosomal protein-like |
| | | | | | | | [Arabidopsis thaliana] |
| 1175 | LIB3602-043-Q6-K1-C11 | 7413634 | BLASTX | 342 | 6.00E−32 | 72 | (AL162971) 60S ribosomal protein-like |
| | | | | | | | [Arabidopsis thaliana] |
| 1176 | LIB3602-034-Q6-K1-D7 | 7413634 | BLASTX | 272 | 6.00E−24 | 76 | (AL162971) 60S ribosomal protein-like |
| | | | | | | | [Arabidopsis thaliana] |
| 1177 | LIB3602-094-Q6-K6-E8 | 7413634 | BLASTX | 219 | 1.00E−17 | 86 | (AL162971) 60S ribosomal protein-like |
| | | | | | | | [Arabidopsis thaliana] |
| 1178 | LIB3602-030-Q6-K1-H12 | 7486582 | BLASTX | 229 | 9.00E−19 | 43 | hypothetical protein F7H19.70 - Arabidopsis |
| | | | | | | | thaliana >gi\|3292814\|emb\|CAA19804.1\| |
| | | | | | | | (AL031018) putative protein [Arabidopsis thaliana] |
| | | | | | | | >gi\|7269136\|emb\|CAB79244.1\| (AL161558) |
| | | | | | | | putative protein [Arabidopsis thaliana] |
| 1179 | LIB3602-105-Q1-K1-H3 | 7486582 | BLASTX | 224 | 5.00E−18 | 50 | hypothetical protein F7H19.70 - Arabidopsis |
| | | | | | | | thaliana >gi\|3292814\|emb\|CAA19804.1\| |
| | | | | | | | (AL031018) putative protein [Arabidopsis thaliana] |
| | | | | | | | >gi\|7269136\|emb\|CAB79244.1\| (AL161558) |
| | | | | | | | putative protein [Arabidopsis thaliana] |
| 1180 | LIB3602-058-Q6-K1-E5 | 7486582 | BLASTX | 223 | 5.00E−18 | 41 | hypothetical protein F7H19.70 - Arabidopsis |
| | | | | | | | thaliana >gi\|3292814\|emb\|CAA19804.1\| |
| | | | | | | | (AL031018) putative protein [Arabidopsis thaliana] |
| | | | | | | | >gi\|7269136\|emb\|CAB79244.1\| (AL161558) |
| | | | | | | | putative protein [Arabidopsis thaliana] |
| 1181 | LIB3602-114-Q1-K1-H7 | 7486582 | BLASTX | 217 | 3.00E−17 | 35 | hypothetical protein F7H19.70 - Arabidopsis |
| | | | | | | | thaliana >gi\|3292814\|emb\|CAA19804.1\| |
| | | | | | | | (AL031018) putative protein [Arabidopsis thaliana] |
| | | | | | | | >gi\|7269136\|emb\|CAB79244.1\| (AL161558) |
| | | | | | | | putative protein [Arabidopsis thaliana] |
| 1182 | LIB3602-084-Q6-K1-G8 | 7486582 | BLASTX | 190 | 4.00E−14 | 58 | hypothetical protein F7H19.70 - Arabidopsis |
| | | | | | | | thaliana >gi\|3292814\|emb\|CAA19804.1\| |
| | | | | | | | (AL031018) putative protein [Arabidopsis thaliana] |
| | | | | | | | >gi\|7269136\|emb\|CAB79244.1\| (AL161558) |
| | | | | | | | putative protein [Arabidopsis thaliana] |
| 1183 | LIB3602-062-Q6-K1-A2 | 7486582 | BLASTX | 164 | 4.00E−11 | 56 | hypothetical protein F7H19.70 - Arabidopsis |
| | | | | | | | thaliana >gi\|3292814\|emb\|CAA19804.1\| |
| | | | | | | | (AL031018) putative protein [Arabidopsis thaliana] |
| | | | | | | | >gi\|7269136\|emb\|CAB79244.1\| (AL161558) |
| | | | | | | | putative protein [Arabidopsis thaliana] |
| 1184 | LIB3602-068-Q1-K1-F1 | 7486992 | BLASTX | 316 | 9.00E−29 | 42 | hypothetical protein T13L16.5 - Arabidopsis |
| | | | | | | | thaliana >gi\|6598526\|gb\|AAD20114.2\| (AC006201) |
| | | | | | | | hypothetical protein [Arabidopsis thaliana] |
| 1185 | LIB3602-009-Q6-K1-F7 | 7486992 | BLASTX | 244 | 2.00E−20 | 38 | hypothetical protein T13L16.5 - Arabidopsis |
| | | | | | | | thaliana >gi\|6598526\|gb\|AAD20114.2\| (AC006201) |
| | | | | | | | hypothetical protein [Arabidopsis thaliana] |
| 1186 | LIB3602-057-Q6-K1-F3 | 7486992 | BLASTX | 185 | 3.00E−18 | 35 | hypothetical protein T13L16.5 - Arabidopsis |
| | | | | | | | thaliana >gi\|6598526\|gb\|AAD20114.2\| (AC006201) |
| | | | | | | | hypothetical protein [Arabidopsis thaliana] |
| 1187 | LIB3602-085-Q6-K1-C5 | 7486992 | BLASTX | 218 | 2.00E−17 | 35 | hypothetical protein T13L16.5 - Arabidopsis |
| | | | | | | | thaliana >gi\|6598526\|gb\|AAD20114.2\| (AC006201) |
| | | | | | | | hypothetical protein [Arabidopsis thaliana] |
| 1188 | LIB3602-086-Q6-K1-C6 | 7486992 | BLASTX | 193 | 2.00E−14 | 34 | hypothetical protein T13L16.5 - Arabidopsis |
| | | | | | | | thaliana >gi\|6598526\|gb\|AAD20114.2\| (AC006201) |
| | | | | | | | hypothetical protein [Arabidopsis thaliana] |
| 1189 | LIB3602-089-Q6-K6-H12 | 7486992 | BLASTX | 177 | 1.00E−12 | 36 | hypothetical protein T13L16.5 - Arabidopsis |
| | | | | | | | thaliana >gi\|6598526\|gb\|AAD20114.2\| (AC006201) |
| | | | | | | | hypothetical protein [Arabidopsis thaliana] |
| 1190 | LIB3602-070-Q1-K1-B2 | 7441117 | BLASTX | 766 | 1.00E−81 | 77 | ribosomal protein S3a, cytosolic - Arabidopsis |
| | | | | | | | thaliana >gi\|2661422\|emb\|CAA04689.1\| |
| | | | | | | | (AJ001342) Putative S-phase-specific ribosomal |
| | | | | | | | protein [Arabidopsis thaliana] |
| | | | | | | | >gi\|3096936\|emb\|CAA18846.1\| (AL023094) |
| | | | | | | | Putative S-phase-specific ribosomal protein |
| | | | | | | | [Arabidops |
| 1191 | LIB3602-043-Q6-K1-A9 | 7441117 | BLASTX | 654 | 2.00E−68 | 78 | ribosomal protein S3a, cytosolic - Arabidopsis |
| | | | | | | | thaliana >gi\|2661422\|emb\|CAA04689.1\| |
| | | | | | | | (AJ001342) Putative S-phase-specific ribosomal |
| | | | | | | | protein [Arabidopsis thaliana] |
| | | | | | | | >gi\|3096936\|emb\|CAA18846.1\| (AL023094) |
| | | | | | | | Putative S-phase-specific ribosomal protein |
| | | | | | | | [Arabidops |
| 1192 | LIB3602-081-Q6-K6-H1 | 7441117 | BLASTX | 633 | 6.00E−66 | 71 | ribosomal protein S3a, cytosolic - Arabidopsis |
| | | | | | | | thaliana >gi\|2661422\|emb\|CAA04689.1\| |
| | | | | | | | (AJ001342) Putative S-phase-specific ribosomal |
| | | | | | | | protein [Arabidopsis thaliana] |
| | | | | | | | >gi\|3096936\|emb\|CAA18846.1\| (AL023094) |
| | | | | | | | Putative S-phase-specific ribosomal protein |
| | | | | | | | [Arabidops |
| 1193 | LIB3602-070-Q1-K1-G11 | 7441117 | BLASTX | 600 | 4.00E−62 | 71 | ribosomal protein S3a, cytosolic - Arabidopsis |
| | | | | | | | thaliana >gi\|2661422\|emb\|CAA04689.1\| |
| | | | | | | | (AJ001342) Putative S-phase-specific ribosomal |
| | | | | | | | protein [Arabidopsis thaliana] |
| | | | | | | | >gi\|3096936\|emb\|CAA18846.1\| (AL023094) |
| | | | | | | | Putative S-phase-specific ribosomal protein |
| | | | | | | | [Arabidops |
| 1194 | LIB3602-044-Q6-K1-H3 | 7441117 | BLASTX | 520 | 8.00E−53 | 81 | ribosomal protein S3a, cytosolic - Arabidopsis |
| | | | | | | | thaliana >gi\|2661422\|emb\|CAA04689.1\| |
| | | | | | | | (AJ001342) Putative S-phase-specific ribosomal |
| | | | | | | | protein [Arabidopsis thaliana] |
| | | | | | | | >gi\|3096936\|emb\|CAA18846.1\| (AL023094) |
| | | | | | | | Putative S-phase-specific ribosomal protein |
| | | | | | | | [Arabidops |
| 1195 | LIB3602-054-Q6-K1-F8 | 7441117 | BLASTX | 473 | 2.00E−47 | 81 | ribosomal protein S3a, cytosolic - Arabidopsis |
| | | | | | | | thaliana >gi\|2661422\|emb\|CAA04689.1\| |
| | | | | | | | (AJ001342) Putative S-phase-specific ribosomal |
| | | | | | | | protein [Arabidopsis thaliana] |
| | | | | | | | >gi\|3096936\|emb\|CAA18846.1\| (AL023094) |
| | | | | | | | Putative S-phase-specific ribosomal protein |
| | | | | | | | [Arabidops |
| 1196 | LIB3602-046-Q6-K1-C2 | 1710530 | BLASTX | 577 | 2.00E−59 | 71 | 60S RIBOSOMAL PROTEIN L27A |
| | | | | | | | >gi\|2129719\|pir\|\|S71256 ribosomal protein L27a, |
| | | | | | | | cytosolic - Arabidopsis thaliana |
| | | | | | | | >gi\|1107487\|emb\|CAA63025.1\| (X91959) 60S |
| | | | | | | | ribosomal protein L27a [Arabidopsis thaliana] |
| | | | | | | | >gi\|6175150\|gb\|AAF04877.1\|AC010796_{—13} |
| | | | | | | | (AC010796) 60S ribosom |
| 1197 | LIB3602-049-Q6-K1-E3 | 1710530 | BLASTX | 563 | 8.00E−58 | 71 | 60S RIBOSOMAL PROTEIN L27A |
| | | | | | | | >gi\|2129719\|pir\|\|S71256 ribosomal protein L27a, |
| | | | | | | | cytosolic - Arabidopsis thaliana |
| | | | | | | | >gi\|1107487\|emb\|CAA63025.1\| (X91959) 60S |
| | | | | | | | ribosomal protein L27a [Arabidopsis thaliana] |
| | | | | | | | >gi\|6175150\|gb\|AAF04877.1\|AC010796_13 |
| | | | | | | | (AC010796) 60S ribosom |
| 1198 | LIB3602-062-Q6-K1-B1 | 1710530 | BLASTX | 339 | 1.00E−31 | 62 | 60S RIBOSOMAL PROTEIN L27A |
| | | | | | | | >gi\|2129719\|pir\|\|S71256 ribosomal protein L27a, |
| | | | | | | | cytosolic - Arabidopsis thaliana |
| | | | | | | | >gi\|1107487\|emb\|CAA63025.1\| (X91959) 60S |
| | | | | | | | ribosomal protein L27a [Arabidopsis thaliana] |
| | | | | | | | >gi\|6175150\|gb\|AAF04877.1\|AC010796_13 |
| | | | | | | | (AC010796) 60S ribosom |
| 1199 | LIB3602-120-Q1-K1-F11 | 1710530 | BLASTX | 160 | 3.00E−22 | 68 | 60S RIBOSOMAL PROTEIN L27A |
| | | | | | | | >gi\|2129719\|pir\|\|S71256 ribosomal protein L27a, |
| | | | | | | | cytosolic - Arabidopsis thaliana |
| | | | | | | | >gi\|1107487\|emb\|CAA63025.1\| (X91959) 60S |
| | | | | | | | ribosomal protein L27a [Arabidopsis thaliana] |
| | | | | | | | >gi\|6175150\|gb\|AAF04877.1\|AC010796_13 |
| | | | | | | | (AC010796) 60S ribosom |
| 1200 | LIB3602-113-Q1-K1-F11 | 1710530 | BLASTX | 258 | 4.00E−22 | 56 | 60S RIBOSOMAL PROTEIN L27A |
| | | | | | | | >gi\|2129719\|pir\|\|S71256 ribosomal protein L27a, |
| | | | | | | | cytosolic - Arabidopsis thaliana |
| | | | | | | | >gi\|1107487\|emb\|CAA63025.1\| (X91959) 60S |
| | | | | | | | ribosomal protein L27a [Arabidopsis thaliana] |
| | | | | | | | >gi\|6175150\|gb\|AAF04877.1\|AC010796_13 |
| | | | | | | | (AC010796) 60S ribosom |
| 1201 | LIB3602-119-Q1-K1-B11 | 1710530 | BLASTX | 189 | 1.00E−14 | 71 | 60S RIBOSOMAL PROTEIN L27A |
| | | | | | | | >gi\|2129719\|pir\|\|S71256 ribosomal protein L27a, |
| | | | | | | | cytosolic - Arabidopsis thaliana |
| | | | | | | | >gi\|1107487\|emb\|CAA63025.1\| (X91959) 60S |
| | | | | | | | ribosomal protein L27a [Arabidopsis thaliana] |
| | | | | | | | >gi\|6175150\|gb\|AAF04877.1\|AC010796_13 |
| | | | | | | | (AC010796) 60S ribosom |
| 1202 | LIB3602-077-Q6-K6-E7 | 4139222 | BLASTX | 353 | 4.00E−33 | 59 | (AF104633) light harvesting complex I protein |
| | | | | | | | precursor [Chlamydomonas reinhardtii] |
| 1203 | LIB3602-076-Q6-K6-G3 | 4139222 | BLASTX | 344 | 4.00E−32 | 56 | (AF104633) light harvesting complex I protein |
| | | | | | | | precursor [Chlamydomonas reinhardtii] |
| 1204 | LIB3602-048-Q6-K1QA-H3 | 4139222 | BLASTX | 299 | 6.00E−27 | 57 | (AF104633) light harvesting complex I protein |
| | | | | | | | precursor [Chlamydomonas reinhardtii] |
| 1205 | LIB3602-113-Q1-K1-A4 | 4139222 | BLASTX | 208 | 1.00E−17 | 72 | (AF104633) light harvesting complex I protein |
| | | | | | | | precursor [Chlamydomonas reinhardtii] |
| 1206 | LIB3602-013-Q6-K1-H4 | 4139222 | BLASTX | 213 | 7.00E−17 | 63 | (AF104633) light harvesting complex I protein |
| | | | | | | | precursor [Chlamydomonas reinhardtii] |
| 1207 | LIB3602-105-Q1-K1-E12 | 4139222 | BLASTX | 181 | 2.00E−16 | 70 | (AF104633) light harvesting complex I protein |
| | | | | | | | precursor [Chlamydomonas reinhardtii] |
| 1208 | LIB3602-011-Q6-K1-B4 | 5758041 | BLASTX | 303 | 3.00E−27 | 49 | (AF133814) ferritin 1 [Solanum tuberosum] |
| 1209 | LIB3602-018-Q6-K1-A6 | 5758041 | BLASTX | 303 | 3.00E−27 | 49 | (AF133814) ferritin 1 [Solanum tuberosum] |
| 1210 | LIB3602-001-P1-K6-B3 | 5758041 | BLASTX | 303 | 3.00E−27 | 49 | (AF133814) ferritin 1 [Solanum tuberosum] |
| 1211 | LIB3602-014-Q6-K1-A11 | 5758041 | BLASTX | 295 | 2.00E−26 | 50 | (AF133814) ferritin 1 [Solanum tuberosum] |
| 1212 | LIB3602-107-Q1-K1-E12 | 5758041 | BLASTX | 275 | 5.00E−24 | 49 | (AF133814) ferritin 1 [Solanum tuberosum] |
| 1213 | LIB3602-051-Q6-K1-D11 | 5758041 | BLASTX | 148 | 3.00E−21 | 45 | (AF133814) ferritin 1 [Solanum tuberosum] |
| 1214 | LIB3602-020-Q6-K1-H3 | 3122724 | BLASTX | 289 | 6.00E−26 | 81 | 60S RIBOSOMAL PROTEIN L38 |
| | | | | | | | >gi\|2289009\|gb\|AAB64338.1\| (AC002335) 60S |
| | | | | | | | ribosomal protein L38 [Arabidopsis thaliana] |
| | | | | | | | >gi\|6996290\|emb\|CAB75451.1\| (AL138659) 60S |
| | | | | | | | RIBOSOMAL PROTEIN L38-like protein |
| | | | | | | | [Arabidopsis thaliana] |
| 1215 | LIB3602-072-Q1-K1-E10 | 3122724 | BLASTX | 283 | 3.00E−25 | 79 | 60S RIBOSOMAL PROTEIN L38 |
| | | | | | | | >gi\|2289009\|gb\|AAB64338.1\| (AC002335) 60S |
| | | | | | | | ribosomal protein L38 [Arabidopsis thaliana] |
| | | | | | | | >gi\|6996290\|emb\|CAB75451.1\| (AL138659) 60S |
| | | | | | | | RIBOSOMAL PROTEIN L38-like protein |
| | | | | | | | [Arabidopsis thaliana] |
| 1216 | LIB3602-100-Q1-K1-A5 | 3122724 | BLASTX | 158 | 1.00E−20 | 79 | 60S RIBOSOMAL PROTEIN L38 |
| | | | | | | | >gi\|2289009\|gb\|AAB64338.1\| (AC002335) 60S |
| | | | | | | | ribosomal protein L38 [Arabidopsis thaliana] |
| | | | | | | | >gi\|6996290\|emb\|CAB75451.1\| (AL138659) 60S |
| | | | | | | | RIBOSOMAL PROTEIN L38-like protein |
| | | | | | | | [Arabidopsis thaliana] |
| 1217 | LIB3602-067-Q1-K1-A12 | 3122724 | BLASTX | 244 | 2.00E−20 | 79 | 60S RIBOSOMAL PROTEIN L38 |
| | | | | | | | >gi\|2289009\|gb\|AAB64338.1\| (AC002335) 60S |
| | | | | | | | ribosomal protein L38 [Arabidopsis thaliana] |
| | | | | | | | >gi\|6996290\|emb\|CAB75451.1\| (AL138659) 60S |
| | | | | | | | RIBOSOMAL PROTEIN L38-like protein |
| | | | | | | | [Arabidopsis thaliana] |
| 1218 | LIB3602-108-Q1-K1-A5 | 3122724 | BLASTX | 148 | 2.00E−19 | 81 | 60S RIBOSOMAL PROTEIN L38 |
| | | | | | | | >gi\|2289009\|gb\|AAB64338.1\| (AC002335) 60S |
| | | | | | | | ribosomal protein L38 [Arabidopsis thaliana] |
| | | | | | | | >gi\|6996290\|emb\|CAB75451.1\| (AL138659) 60S |
| | | | | | | | RIBOSOMAL PROTEIN L38-like protein |
| | | | | | | | [Arabidopsis thaliana] |
| 1219 | LIB3602-020-Q6-K1-G5 | 3122724 | BLASTX | 155 | 1.00E−10 | 82 | 60S RIBOSOMAL PROTEIN L38 |
| | | | | | | | >gi\|2289009\|gb\|AAB64338.1\| (AC002335) 60S |
| | | | | | | | ribosomal protein L38 [Arabidopsis thaliana] |
| | | | | | | | >gi\|6996290\|emb\|CAB75451.1\| (AL138659) 60S |
| | | | | | | | RIBOSOMAL PROTEIN L38-like protein |
| | | | | | | | [Arabidopsis thaliana] |
| 1220 | LIB3602-066-Q1-K6-H8 | 132100 | BLASTX | 570 | 1.00E−58 | 66 | RIBULOSE BISPHOSPHATE CARBOXYLASE |
| | | | | | | | SMALL CHAIN 3 PRECURSOR (RUBISCO |
| | | | | | | | SMALL SUBUNIT 3) >gi\|68108\|pir\|\|RKJK3C |
| | | | | | | | ribulose-bisphosphate carboxylase (EC 4.1.1.39) |
| | | | | | | | small chain 3 precursor - Acetabularia cliftonii |
| | | | | | | | >gi\|732546\|emb\|CAA36105.1\| (X51808) ribulose |
| | | | | | | | bisphos |
| 1221 | LIB3602-049-Q6-K1-G9 | 132100 | BLASTX | 556 | 5.00E−57 | 66 | RIBULOSE BISPHOSPHATE CARBOXYLASE |
| | | | | | | | SMALL CHAIN 3 PRECURSOR (RUBISCO |
| | | | | | | | SMALL SUBUNIT 3) >gi\|68108\|pir\|\|RKJK3C |
| | | | | | | | ribulose-bisphosphate carboxylase (EC 4.1.1.39) |
| | | | | | | | small chain 3 precursor - Acetabularia cliftonii |
| | | | | | | | >gi\|732546\|emb\|CAA36105.1\| (X51808) ribulose |
| | | | | | | | bisphos |
| 1222 | LIB3602-012-Q6-K1-G3 | 132100 | BLASTX | 546 | 9.00E−56 | 68 | RIBULOSE BISPHOSPHATE CARBOXYLASE |
| | | | | | | | SMALL CHAIN 3 PRECURSOR (RUBISCO |
| | | | | | | | SMALL SUBUNIT 3) >gi\|68108\|pir\|\|RKJK3C |
| | | | | | | | ribulose-bisphosphate carboxylase (EC 4.1.1.39) |
| | | | | | | | small chain 3 precursor - Acetabularia cliftonii |
| | | | | | | | >gi\|732546\|emb\|CAA36105.1\| (X51808) ribulose |
| | | | | | | | bisphos |
| 1223 | LIB3602-005-Q1-K1-E10 | 132100 | BLASTX | 520 | 1.00E−52 | 68 | RIBULOSE BISPHOSPHATE CARBOXYLASE |
| | | | | | | | SMALL CHAIN 3 PRECURSOR (RUBISCO |
| | | | | | | | SMALL SUBUNIT 3) >gi\|68108\|pir\|\|RKJK3C |
| | | | | | | | ribulose-bisphosphate carboxylase (EC 4.1.1.39) |
| | | | | | | | small chain 3 precursor - Acetabularia cliftonii |
| | | | | | | | >gi\|732546\|emb\|CAA36105.1\| (X51808) ribulose |
| | | | | | | | bisphos |
| 1224 | LIB3602-100-Q1-K1-A12 | 132100 | BLASTX | 502 | 1.00E−50 | 69 | RIBULOSE BISPHOSPHATE CARBOXYLASE |
| | | | | | | | SMALL CHAIN 3 PRECURSOR (RUBISCO |
| | | | | | | | SMALL SUBUNIT 3) >gi\|68108\|pir\|\|RKJK3C |
| | | | | | | | ribulose-bisphosphate carboxylase (EC 4.1.1.39) |
| | | | | | | | small chain 3 precursor - Acetabularia cliftonii |
| | | | | | | | >gi\|732546\|emb\|CAA36105.1\| (X51808) ribulose |
| | | | | | | | bisphos |
| 1225 | LIB3602-018-Q6-K1-F5 | 132100 | BLASTX | 454 | 5.00E−45 | 68 | RIBULOSE BISPHOSPHATE CARBOXYLASE |
| | | | | | | | SMALL CHAIN 3 PRECURSOR (RUBISCO |
| | | | | | | | SMALL SUBUNIT 3) >gi\|68108\|pir\|\|RKJK3C |
| | | | | | | | ribulose-bisphosphate carboxylase (EC 4.1.1.39) |
| | | | | | | | small chain 3 precursor - Acetabularia cliftonii |
| | | | | | | | >gi\|732546\|emb\|CAA36105.1\| (X51808) ribulose |
| | | | | | | | bisphos |
| 1226 | LIB3602-017-Q6-K1-A9 | 1709786 | BLASTX | 377 | 5.00E−36 | 55 | PYRROLINE-5-CARBOXYLATE REDUCTASE |
| | | | | | | | (P5CR) (P5C REDUCTASE) |
| | | | | | | | >gi\|541894\|pir\|\|JQ2334 pyrroline-5-carboxylate |
| | | | | | | | reductase (EC 1.5.1.2) - Arabidopsis thaliana |
| | | | | | | | >gi\|166815\|gb\|AAA61346.1\| (M76538) pyrroline |
| | | | | | | | carboxylate reductase [Arabidopsis thaliana] |
| | | | | | | | >gi\|1632776\|emb |
| 1227 | LIB3602-006-Q1-K1-A2 | 1709786 | BLASTX | 369 | 5.00E−35 | 68 | PYRROLINE-5-CARBOXYLATE REDUCTASE |
| | | | | | | | (P5CR) (P5C REDUCTASE) |
| | | | | | | | >gi\|541894\|pir\|\|JQ2334 pyrroline-5-carboxylate |
| | | | | | | | reductase (EC 1.5.1.2) - Arabidopsis thaliana |
| | | | | | | | >gi\|166815\|gb\|AAA61346.1\| (M76538) pyrroline |
| | | | | | | | carboxylate reductase [Arabidopsis thaliana] |
| | | | | | | | >gi\|1632776\|emb |
| 1228 | LIB3602-106-Q1-K1-C9 | 1709786 | BLASTX | 288 | 2.00E−25 | 55 | PYRROLINE-5-CARBOXYLATE REDUCTASE |
| | | | | | | | (P5CR) (P5C REDUCTASE) |
| | | | | | | | >gi\|541894\|pir\|\|JQ2334 pyrroline-5-carboxylate |
| | | | | | | | reductase (EC 1.5.1.2) - Arabidopsis thaliana |
| | | | | | | | >gi\|166815\|gb\|AAA61346.1\| (M76538) pyrroline |
| | | | | | | | carboxylate reductase [Arabidopsis thaliana] |
| | | | | | | | >gi\|1632776\|emb |
| 1229 | LIB3602-060-Q6-K1-C1 | 1709786 | BLASTX | 230 | 4.00E−19 | 61 | PYRROLINE-5-CARBOXYLATE REDUCTASE |
| | | | | | | | (P5CR) (P5C REDUCTASE) |
| | | | | | | | >gi\|541894\|pir\|\|JQ2334 pyrroline-5-carboxylate |
| | | | | | | | reductase (EC 1.5.1.2) - Arabidopsis thaliana |
| | | | | | | | >gi\|166815\|gb\|AAA61346.1\| (M76538) pyrroline |
| | | | | | | | carboxylate reductase [Arabidopsis thaliana] |
| | | | | | | | >gi\|1632776\|emb |
| 1230 | LIB3602-046-Q6-K1-F7 | 1709786 | BLASTX | 206 | 5.00E−16 | 67 | PYRROLINE-5-CARBOXYLATE REDUCTASE |
| | | | | | | | (P5CR) (P5C REDUCTASE) |
| | | | | | | | >gi\|541894\|pir\|\|JQ2334 pyrroline-5-carboxylate |
| | | | | | | | reductase (EC 1.5.1.2) - Arabidopsis thaliana |
| | | | | | | | >gi\|166815\|gb\|AAA61346.1\| (M76538) pyrroline |
| | | | | | | | carboxylate reductase [Arabidopsis thaliana] |
| | | | | | | | >gi\|1632776\|emb |
| 1231 | LIB3602-007-Q1-K1-G11 | 1709786 | BLASTX | 158 | 3.00E−10 | 34 | PYRROLINE-5-CARBOXYLATE REDUCTASE |
| | | | | | | | (P5CR) (P5C REDUCTASE) |
| | | | | | | | >gi\|541894\|pir\|\|JQ2334 pyrroline-5-carboxylate |
| | | | | | | | reductase (EC 1.5.1.2) - Arabidopsis thaliana |
| | | | | | | | >gi\|166815\|gb\|AAA61346.1\| (M76538) pyrroline |
| | | | | | | | carboxylate reductase [Arabidopsis thaliana] |
| | | | | | | | >gi\|1632776\|emb |
| 1232 | LIB3602-017-Q6-K1-B10 | 4590398 | BLASTX | 387 | 3.00E−37 | 71 | (AF124243) ribosomal protein homolog |
| | | | | | | | [Cryptosporidium parvum] |
| 1233 | LIB3602-109-Q1-K1-G11 | 4590398 | BLASTX | 207 | 7.00E−31 | 68 | (AF124243) ribosomal protein homolog |
| | | | | | | | [Cryptosporidium parvum] |
| 1234 | LIB3602-109-Q1-K1-D2 | 4590398 | BLASTX | 218 | 5.00E−29 | 70 | (AF124243) ribosomal protein homolog |
| | | | | | | | [Cryptosporidium parvum] |
| 1235 | LIB3602-006-Q1-K1-F4 | 4590398 | BLASTX | 309 | 3.00E−28 | 65 | (AF124243) ribosomal protein homolog |
| | | | | | | | [Cryptosporidium parvum] |
| 1236 | LIB3602-009-Q6-K1-A8 | 4590398 | BLASTX | 302 | 2.00E−27 | 69 | (AF124243) ribosomal protein homolog |
| | | | | | | | [Cryptosporidium parvum] |
| 1237 | LIB3602-049-Q6-K1-C4 | 1168732 | BLASTX | 378 | 4.00E−36 | 60 | CINNAMYL-ALCOHOL DEHYDROGENASE |
| | | | | | | | (CAD) >gi\|421914\|pir\|\|S28045 cinnamyl-alcohol |
| | | | | | | | dehydrogenase (EC 1.1.1.195) ELI3 - parsley |
| | | | | | | | (fragment) >gi\|836638\|emb\|CAA48028.1\| (X67817) |
| | | | | | | | Eli3 [Petroselinum crispum] |
| 1238 | LIB3602-038-Q6-K1-C5 | 1168732 | BLASTX | 249 | 1.00E−34 | 57 | CINNAMYL-ALCOHOL DEHYDROGENASE |
| | | | | | | | (CAD) >gi\|421914\|pir\|\|S28045 cinnamyl-alcohol |
| | | | | | | | dehydrogenase (EC 1.1.1.195) ELI3 - parsley |
| | | | | | | | (fragment) >gi\|836638\|emb\|CAA48028.1\| (X67817) |
| | | | | | | | Eli3 [Petroselinum crispum] |
| 1239 | LIB3602-038-Q6-K1-C4 | 1168732 | BLASTX | 249 | 1.00E−34 | 57 | CINNAMYL-ALCOHOL DEHYDROGENASE |
| | | | | | | | (CAD) >gi\|421914\|pir\|\|S28045 cinnamyl-alcohol |
| | | | | | | | dehydrogenase (EC 1.1.1.195) ELI3 - parsley |
| | | | | | | | (fragment) >gi\|836638\|emb\|CAA48028.1\| (X67817) |
| | | | | | | | Eli3 [Petroselinum crispum] |
| 1240 | LIB3602-067-Q1-K1-A6 | 1168732 | BLASTX | 304 | 2.00E−27 | 56 | CINNAMYL-ALCOHOL DEHYDROGENASE |
| | | | | | | | (CAD) >gi\|421914\|pir\|\|S28045 cinnamyl-alcohol |
| | | | | | | | dehydrogenase (EC 1.1.1.195) ELI3 - parsley |
| | | | | | | | (fragment) >gi\|836638\|emb\|CAA48028.1\| (X67817) |
| | | | | | | | Eli3 [Petroselinum crispum] |
| 1241 | LIB3602-001-P1-K6-B6 | 1168732 | BLASTX | 301 | 4.00E−27 | 57 | CINNAMYL-ALCOHOL DEHYDROGENASE |
| | | | | | | | (CAD) >gi\|421914\|pir\|\|S28045 cinnamyl-alcohol |
| | | | | | | | dehydrogenase (EC 1.1.1.195) ELI3 - parsley |
| | | | | | | | (fragment) >gi\|836638\|emb\|CAA48028.1\| (X67817) |
| | | | | | | | Eli3 [Petroselinum crispum] |
| 1242 | LIB3602-006-Q1-K1-D4 | 7443234 | BLASTX | 364 | 2.00E−34 | 50 | light harvesting complex A protein precursor - |
| | | | | | | | Volvox carteri >gi\|732534\|gb\|AAB40979.1\| |
| | | | | | | | (U22329) light harvesting complex a [Volvox |
| | | | | | | | carteri] |
| 1243 | LIB3602-090-Q6-K6-E6 | 7443234 | BLASTX | 335 | 5.00E−31 | 52 | light harvesting complex A protein precursor - |
| | | | | | | | Volvox carteri >gi\|732534\|gb\|AAB40979.1\| |
| | | | | | | | (U22329) light harvesting complex a [Volvox |
| | | | | | | | carteri] |
| 1244 | LIB3602-070-Q1-K1-G4 | 7443234 | BLASTX | 237 | 4.00E−21 | 52 | light harvesting complex A protein precursor - |
| | | | | | | | Volvox carteri >gi\|732534\|gb\|AAB40979.1\| |
| | | | | | | | (U22329) light harvesting complex a [Volvox |
| | | | | | | | carteri] |
| 1245 | LIB3602-091-Q6-K6-E1 | 7443234 | BLASTX | 230 | 3.00E−19 | 52 | light harvesting complex A protein precursor - |
| | | | | | | | Volvox carteri >gi\|732534\|gb\|AAB40979.1\| |
| | | | | | | | (U22329) light harvesting complex a [Volvox |
| | | | | | | | carteri] |
| 1246 | LIB3602-105-Q1-K1-A9 | 7443234 | BLASTX | 225 | 4.00E−18 | 62 | light harvesting complex A protein precursor - |
| | | | | | | | Volvox carteri >gi\|732534\|gb\|AAB40979.1\| |
| | | | | | | | (U22329) light harvesting complex a [Volvox |
| | | | | | | | carteri] |
| 1247 | LIB3602-015-Q6-K1-A1 | 7430859 | BLASTX | 228 | 1.00E−18 | 47 | glutaredoxin - rice >gi\|2114207\|dbj\|BAA20071.1\| |
| | | | | | | | (D86744) glutaredoxin [Oryza sativa] |
| 1248 | LIB3602-068-Q1-K1-D5 | 7430859 | BLASTX | 228 | 1.00E−18 | 47 | glutaredoxin - rice >gi\|2114207\|dbj\|BAA20071.1\| |
| | | | | | | | (D86744) glutaredoxin [Oryza sativa] |
| 1249 | LIB3602-006-Q1-K1-G7 | 7430859 | BLASTX | 228 | 2.00E−18 | 47 | glutaredoxin - rice >gi\|2114207\|dbj\|BAA20071.1\| |
| | | | | | | | (D86744) glutaredoxin [Oryza sativa] |
| 1250 | LIB3602-079-Q6-K6-E5 | 7430859 | BLASTX | 228 | 2.00E−18 | 47 | glutaredoxin - rice >gi\|2114207\|dbj\|BAA20071.1\| |
| | | | | | | | (D86744) glutaredoxin [Oryza sativa] |
| 1251 | LIB3602-074-Q1-K1-H6 | 7430859 | BLASTX | 152 | 1.00E−11 | 41 | glutaredoxin - rice >gi\|2114207\|dbj\|BAA20071.1\| |
| | | | | | | | (D86744) glutaredoxin [Oryza sativa] |
| 1252 | LIB3602-005-Q1-K1-A8 | 927428 | BLASTX | 658 | 3.00E−71 | 68 | (X86733) fis1 [Linum usitatissimum] |
| 1253 | LIB3602-010-Q6-K1-B9 | 927428 | BLASTX | 671 | 2.00E−70 | 69 | (X86733) fis1 [Linum usitatissimum] |
| 1254 | LIB3602-025-Q6-K1-F4 | 927428 | BLASTX | 487 | 6.00E−49 | 73 | (X86733) fis1 [Linum usitatissimum] |
| 1255 | LIB3602-070-Q1-K1-B7 | 927428 | BLASTX | 487 | 7.00E−49 | 73 | (X86733) fis1 [Linum usitatissimum] |
| 1256 | LIB3602-047-Q6-K1-E12 | 927428 | BLASTX | 431 | 2.00E−42 | 76 | (X86733) fis1 [Linum usitatissimum] |
| 1257 | LIB3602-093-Q6-K6-A5 | 6730739 | BLASTX | 300 | 6.00E−27 | 55 | (AC018849) hypothetical protein [Arabidopsis |
| | | | | | | | thaliana] |
| 1258 | LIB3602-113-Q1-K1-F5 | 6730739 | BLASTX | 300 | 6.00E−27 | 55 | (AC018849) hypothetical protein [Arabidopsis |
| | | | | | | | thaliana] |
| 1259 | LIB3602-015-Q6-K1-H8 | 6730739 | BLASTX | 240 | 4.00E−20 | 47 | (AC018849) hypothetical protein [Arabidopsis |
| | | | | | | | thaliana] |
| 1260 | LIB3602-017-Q6-K1-C5 | 6730739 | BLASTX | 171 | 4.00E−12 | 60 | (AC018849) hypothetical protein [Arabidopsis |
| | | | | | | | thaliana] |
| 1261 | LIB3602-043-Q6-K1-C10 | 6730739 | BLASTX | 145 | 4.00E−09 | 55 | (AC018849) hypothetical protein [Arabidopsis |
| | | | | | | | thaliana] |
| 1262 | LIB3602-042-Q6-K1-F12 | 3075487 | BLASTN | 41 | 2.00E−13 | 91 | Oryza sativa chlorophyll a/b-binding protein |
| | | | | | | | (RCABP69) mRNA, complete cds |
| 1263 | LIB3602-093-Q6-K6-A3 | 3075487 | BLASTN | 41 | 2.00E−13 | 91 | Oryza sativa chlorophyll a/b-binding protein |
| | | | | | | | (RCABP69) mRNA, complete cds |
| 1264 | LIB3602-049-Q6-K1-H11 | 3075487 | BLASTN | 39 | 3.00E−12 | 91 | Oryza sativa chlorophyll a/b-binding protein |
| | | | | | | | (RCABP69) mRNA, complete cds |
| 1265 | LIB3602-056-Q6-K1-G10 | 3075487 | BLASTN | 37 | 5.00E−11 | 90 | Oryza sativa chlorophyll a/b-binding protein |
| | | | | | | | (RCABP69) mRNA, complete cds |
| 1266 | LIB3602-104-Q1-K1-A3 | 3075487 | BLASTN | 34 | 3.00E−09 | 90 | Oryza sativa chlorophyll a/b-binding protein |
| | | | | | | | (RCABP69) mRNA, complete cds |
| 1267 | LIB3602-039-Q6-K1-G4 | 100524 | BLASTX | 768 | 8.00E−82 | 98 | polyubiquitin 6 - common sunflower |
| | | | | | | | >gi\|18824\|emb\|CAA40324.1\| (X57004) |
| | | | | | | | hexaubiquitin protein [Helianthus annuus] |
| | | | | | | | >gi\|1204096\|emb\|CAA40325.1\| (X57005) |
| | | | | | | | hexaubiquitin protein [Helianthus annuus] |
| 1268 | LIB3602-112-Q1-K1-A9 | 100524 | BLASTX | 700 | 7.00E−74 | 96 | polyubiquitin 6 - common sunflower |
| | | | | | | | >gi\|18824\|emb\|CAA40324.1\| (X57004) |
| | | | | | | | hexaubiquitin protein [Helianthus annuus] |
| | | | | | | | >gi\|1204096\|emb\|CAA40325.1\| (X57005) |
| | | | | | | | hexaubiquitin protein [Helianthus annuus] |
| 1269 | LIB3602-023-Q6-K1-C1 | 100524 | BLASTX | 537 | 7.00E−55 | 99 | polyubiquitin 6 - common sunflower |
| | | | | | | | >gi\|18824\|emb\|CAA40324.1\| (X57004) |
| | | | | | | | hexaubiquitin protein [Helianthus annuus] |
| | | | | | | | >gi\|1204096\|emb\|CAA40325.1\| (X57005) |
| | | | | | | | hexaubiquitin protein [Helianthus annuus] |
| 1270 | LIB3602-116-Q1-K1-G10 | 100524 | BLASTX | 226 | 8.00E−36 | 95 | polyubiquitin 6 - common sunflower |
| | | | | | | | >gi\|18824\|emb\|CAA40324.1\| (X57004) |
| | | | | | | | hexaubiquitin protein [Helianthus annuus] |
| | | | | | | | >gi\|1204096\|emb\|CAA40325.1\| (X57005) |
| | | | | | | | hexaubiquitin protein [Helianthus annuus] |
| 1271 | LIB3602-089-Q6-K6-G10 | 100524 | BLASTX | 265 | 3.00E−35 | 98 | polyubiquitin 6 - common sunflower |
| | | | | | | | >gi\|18824\|emb\|CAA40324.1\| (X57004) |
| | | | | | | | hexaubiquitin protein [Helianthus annuus] |
| | | | | | | | >gi\|1204096\|emb\|CAA40325.1\| (X57005) |
| | | | | | | | hexaubiquitin protein [Helianthus annuus] |
| 1272 | LIB3602-071-Q1-K1-D5 | 131382 | BLASTX | 751 | 8.00E−80 | 72 | OXYGEN-EVOLVING ENHANCER PROTEIN 1 |
| | | | | | | | PRECURSOR (OEE1) >gi\|81252\|pir\|\|S05508 |
| | | | | | | | photosystem II oxygen-evolving complex protein 1 |
| | | | | | | | precursor - Chlamydomonas reinhardtii |
| | | | | | | | >gi\|18160\|emb\|CAA32053.1\| (X13826) OEE1 |
| | | | | | | | precursor protein [Chlamydomonas reinhardtii] |
| | | | | | | | >gi\|2286 |
| 1273 | LIB3602-010-Q6-K1-C9 | 131382 | BLASTX | 665 | 8.00E−70 | 72 | OXYGEN-EVOLVING ENHANCER PROTEIN 1 |
| | | | | | | | PRECURSOR (OEE1) >gi\|81252\|pir\|\|S05508 |
| | | | | | | | photosystem II oxygen-evolving complex protein 1 |
| | | | | | | | precursor - Chlamydomonas reinhardtii |
| | | | | | | | >gi\|18160\|emb\|CAA32053.1\| (X13826) OEE1 |
| | | | | | | | precursor protein [Chlamydomonas reinhardtii] |
| | | | | | | | >gi\|2286 |
| 1274 | LIB3602-058-Q6-K1-G8 | 131382 | BLASTX | 647 | 1.00E−67 | 73 | OXYGEN-EVOLVING ENHANCER PROTEIN 1 |
| | | | | | | | PRECURSOR (OEE1) >gi\|81252\|pir\|\|S05508 |
| | | | | | | | photosystem II oxygen-evolving complex protein 1 |
| | | | | | | | precursor - Chlamydomonas reinhardtii |
| | | | | | | | >gi\|18160\|emb\|CAA32053.1\| (X13826) OEE1 |
| | | | | | | | precursor protein [Chlamydomonas reinhardtii] |
| | | | | | | | >gi\|2286 |
| 1275 | LIB3602-043-Q6-K1-F10 | 131382 | BLASTX | 434 | 6.00E−43 | 67 | OXYGEN-EVOLVING ENHANCER PROTEIN 1 |
| | | | | | | | PRECURSOR (OEE1) >gi\|81252\|pir\|\|S05508 |
| | | | | | | | photosystem II oxygen-evolving complex protein 1 |
| | | | | | | | precursor - Chlamydomonas reinhardtii |
| | | | | | | | >gi\|18160\|emb\|CAA32053.1\| (X13826) OEE1 |
| | | | | | | | precursor protein [Chlamydomonas reinhardtii] |
| | | | | | | | >gi\|2286 |
| 1276 | LIB3602-070-Q1-K1-D1 | 131382 | BLASTX | 219 | 2.00E−30 | 64 | OXYGEN-EVOLVING ENHANCER PROTEIN 1 |
| | | | | | | | PRECURSOR (OEE1) >gi\|81252\|pir\|\|S05508 |
| | | | | | | | photosystem II oxygen-evolving complex protein 1 |
| | | | | | | | precursor - Chlamydomonas reinhardtii |
| | | | | | | | >gi\|18160\|emb\|CAA32053.1\| (X13826) OEE1 |
| | | | | | | | precursor protein [Chlamydomonas reinhardtii] |
| | | | | | | | >gi\|2286 |
| 1277 | LIB3602-019-Q6-K1-A7 | 1657621 | BLASTX | 523 | 3.00E−53 | 64 | (U72505) G6p [Arabidopsis thaliana] |
| | | | | | | | >gi\|3068711\|gb\|AAC14411.1\| (AF049236) putative |
| | | | | | | | acyl-coA dehydrogenase [Arabidopsis thaliana] |
| | | | | | | | >gi\|5478795\|dbj\|BAA82478.1\| (AB017643) Short- |
| | | | | | | | chain acyl CoA oxidase [Arabidopsis thaliana] |
| 1278 | LIB3602-019-Q6-K1-A1 | 1657621 | BLASTX | 509 | 1.00E−51 | 64 | (U72505) G6p [Arabidopsis thaliana] |
| | | | | | | | >gi\|3068711\|gb\|AAC14411.1\| (AF049236) putative |
| | | | | | | | acyl-coA dehydrogenase [Arabidopsis thaliana] |
| | | | | | | | >gi\|5478795\|dbj\|BAA82478.1\| (AB017643) Short- |
| | | | | | | | chain acyl CoA oxidase [Arabidopsis thaliana] |
| 1279 | LIB3602-074-Q1-K1-A1 | 1657621 | BLASTX | 318 | 4.00E−29 | 64 | (U72505) G6p [Arabidopsis thaliana] |
| | | | | | | | >gi\|3068711\|gb\|AAC14411.1\| (AF049236) putative |
| | | | | | | | acyl-coA dehydrogenase [Arabidopsis thaliana] |
| | | | | | | | >gi\|5478795\|dbj\|BAA82478.1\| (AB017643) Short- |
| | | | | | | | chain acyl CoA oxidase [Arabidopsis thaliana] |
| 1280 | LIB3602-087-Q6-K1-D2 | 1657621 | BLASTX | 293 | 4.00E−26 | 60 | (U72505) G6p [Arabidopsis thaliana] |
| | | | | | | | >gi\|3068711\|gb\|AAC14411.1\| (AF049236) putative |
| | | | | | | | acyl-coA dehydrogenase [Arabidopsis thaliana] |
| | | | | | | | >gi\|5478795\|dbj\|BAA82478.1\| (AB017643) Short- |
| | | | | | | | chain acyl CoA oxidase [Arabidopsis thaliana] |
| 1281 | LIB3602-040-Q6-K1-E4 | 1657621 | BLASTX | 203 | 1.00E−15 | 68 | (U72505) G6p [Arabidopsis thaliana] |
| | | | | | | | >gi\|3068711\|gb\|AAC14411.1\| (AF049236) putative |
| | | | | | | | acyl-coA dehydrogenase [Arabidopsis thaliana] |
| | | | | | | | >gi\|5478795\|dbj\|BAA82478.1\| (AB017643) Short- |
| | | | | | | | chain acyl CoA oxidase [Arabidopsis thaliana] |
| 1282 | LIB3602-071-Q1-K1-C3 | 1172635 | BLASTX | 799 | 4.00E−90 | 90 | 26S PROTEASE REGULATORY SUBUNIT 4 |
| | | | | | | | HOMOLOG (TAT-BINDING PROTEIN |
| | | | | | | | HOMOLOG 2) >gi\|7435744\|pir\|\|T03776 tat binding |
| | | | | | | | protein homolog - rice >gi\|556558\|dbj\|BAA04615.1\| |
| | | | | | | | (D17789) rice homologue of Tat binding protein |
| | | | | | | | [Oryza sativa] |
| 1283 | LIB3602-049-Q6-K1-A5 | 1172635 | BLASTX | 744 | 5.00E−79 | 86 | 26S PROTEASE REGULATORY SUBUNIT 4 |
| | | | | | | | HOMOLOG (TAT-BINDING PROTEIN |
| | | | | | | | HOMOLOG 2) >gi\|7435744\|pir\|\|T03776 tat binding |
| | | | | | | | protein homolog - rice >gi\|556558\|dbj\|BAA04615.1\| |
| | | | | | | | (D17789) rice homologue of Tat binding protein |
| | | | | | | | [Oryza sativa] |
| 1284 | LIB3602-049-Q6-K1-A6 | 1172635 | BLASTX | 719 | 4.00E−76 | 84 | 26S PROTEASE REGULATORY SUBUNIT 4 |
| | | | | | | | HOMOLOG (TAT-BINDING PROTEIN |
| | | | | | | | HOMOLOG 2) >gi\|7435744\|pir\|\|T03776 tat binding |
| | | | | | | | protein homolog - rice >gi\|556558\|dbj\|\|BAA04615.1\| |
| | | | | | | | (D17789) rice homologue of Tat binding protein |
| | | | | | | | [Oryza sativa] |
| 1285 | LIB3602-106-Q1-K1-H10 | 1172635 | BLASTX | 467 | 1.00E−46 | 72 | 26S PROTEASE REGULATORY SUBUNIT 4 |
| | | | | | | | HOMOLOG (TAT-BINDING PROTEIN |
| | | | | | | | HOMOLOG 2) >gi\|7435744\|pir\|\|T03776 tat binding |
| | | | | | | | protein homolog - rice >gi\|556558\|dbj\|BAA04615.1\| |
| | | | | | | | (D17789) rice homologue of Tat binding protein |
| | | | | | | | [Oryza sativa] |
| 1286 | LIB3602-013-Q6-K1-C6 | 1172635 | BLASTX | 246 | 5.00E−25 | 66 | 26S PROTEASE REGULATORY SUBUNIT 4 |
| | | | | | | | HOMOLOG (TAT-BINDING PROTEIN |
| | | | | | | | HOMOLOG 2) >gi\|7435744\|pir\|\|T03776 tat binding |
| | | | | | | | protein homolog - rice >gi\|556558\|dbj\|BAA04615.1\| |
| | | | | | | | (D17789) rice homologue of Tat binding protein |
| | | | | | | | [Oryza sativa] |
| 1287 | LIB3602-114-Q1-K1-F1 | 131772 | BLASTX | 595 | 1.00E−61 | 85 | 40S RIBOSOMAL PROTEIN S14 (CLONE |
| | | | | | | | MCH1) >gi\|82723\|pir\|\|A30097 ribosomal protein |
| | | | | | | | S14 (clone MCH1) - maize |
| 1288 | LIB3602-039-Q6-K1-F12 | 131772 | BLASTX | 595 | 1.00E−61 | 85 | 40S RIBOSOMAL PROTEIN S14 (CLONE |
| | | | | | | | MCH1) >gi\|82723\|pir\|\|A30097 ribosomal protein |
| | | | | | | | S14 (clone MCH1) - maize |
| 1289 | LIB3602-034-Q6-K1-F7 | 131772 | BLASTX | 488 | 3.00E−49 | 91 | 40S RIBOSOMAL PROTEIN S14 (CLONE |
| | | | | | | | MCH1) >gi\|82723\|pir\|\|A30097 ribosomal protein |
| | | | | | | | S14 (clone MCH1) - maize |
| 1290 | LIB3602-059-Q6-K1-H5 | 131772 | BLASTX | 485 | 1.00E−48 | 90 | 40S RIBOSOMAL PROTEIN S14 (CLONE |
| | | | | | | | MCH1) >gi\|82723\|pir\|\|A30097 ribosomal protein |
| | | | | | | | S14 (clone MCH1) - maize |
| 1291 | LIB3602-035-Q1-K1-F7 | 131772 | BLASTX | 460 | 1.00E−45 | 91 | 40S RIBOSOMAL PROTEIN S14 (CLONE |
| | | | | | | | MCH1) >gi\|82723\|pir\|\|A30097 ribosomal protein |
| | | | | | | | S14 (clone MCH1) - maize |
| 1292 | LIB3602-053-Q6-K1-C3 | 4757371 | BLASTX | 272 | 1.00E−23 | 76 | (AB026686) chlorophyll a/b-binding protein |
| | | | | | | | precursor [Physcomitrella patens] |
| 1293 | LIB3602-102-Q1-K1-G9 | 4757371 | BLASTX | 268 | 3.00E−23 | 85 | (AB026686) chlorophyll a/b-binding protein |
| | | | | | | | precursor [Physcomitrella patens] |
| 1294 | LIB3602-070-Q1-K1-B10 | 4757371 | BLASTX | 268 | 3.00E−23 | 85 | (AB026686) chlorophyll a/b-binding protein |
| | | | | | | | precursor [Physcomitrella patens] |
| 1295 | LIB3602-102-Q1-K1-G10 | 4757371 | BLASTX | 188 | 6.00E−14 | 84 | (AB026686) chlorophyll a/b-binding protein |
| | | | | | | | precursor [Physcomitrella patens] |
| 1296 | LIB3602-084-Q6-K1-C12 | 4757371 | BLASTX | 161 | 6.00E−11 | 69 | (AB026686) chlorophyll a/b-binding protein |
| | | | | | | | precursor [Physcomitrella patens] |
| 1297 | LIB3602-002-P1-K6-A7 | 6015604 | BLASTX | 466 | 1.00E−48 | 62 | (X97322) ribosomal protein L9 [Pisum sativum] |
| 1298 | LIB3602-052-Q6-K1-F7 | 6015604 | BLASTX | 409 | 7.00E−40 | 58 | (X97322) ribosomal protein L9 [Pisum sativum] |
| 1299 | LIB3602-056-Q6-K1-C10 | 6015604 | BLASTX | 399 | 1.00E−38 | 58 | (X97322) ribosomal protein L9 [Pisum sativum] |
| 1300 | LIB3602-020-Q6-K1-F9 | 6015604 | BLASTX | 319 | 2.00E−29 | 60 | (X97322) ribosomal protein L9 [Pisum sativum] |
| 1301 | LIB3602-038-Q6-K1-G9 | 6015604 | BLASTX | 312 | 1.00E−28 | 59 | (X97322) ribosomal protein L9 [Pisum sativum] |
| 1302 | LIB3602-069-Q1-K1-F5 | 7109466 | BLASTX | 481 | 4.00E−48 | 57 | (AC016662) putative 60S ribosomal protein L6 |
| | | | | | | | [Arabidopsis thaliana] |
| 1303 | LIB3602-092-Q6-K6-H4 | 7109466 | BLASTX | 453 | 8.00E−45 | 61 | (AC016662) putative 60S ribosomal protein L6 |
| | | | | | | | [Arabidopsis thaliana] |
| 1304 | LIB3602-027-Q6-K1-B9 | 7109466 | BLASTX | 443 | 9.00E−44 | 58 | (AC016662) putative 60S ribosomal protein L6 |
| | | | | | | | [Arabidopsis thaliana] |
| 1305 | LIB3602-047-Q6-K1-C9 | 7109466 | BLASTX | 432 | 2.00E−42 | 66 | (AC016662) putative 60S ribosomal protein L6 |
| | | | | | | | [Arabidopsis thaliana] |
| 1306 | LIB3602-046-Q6-K1-C9 | 7109466 | BLASTX | 409 | 7.00E−40 | 60 | (AC016662) putative 60S ribosomal protein L6 |
| | | | | | | | [Arabidopsis thaliana] |
| 1307 | LIB3602-030-Q6-K1-E2 | 6225817 | BLASTX | 507 | 2.00E−51 | 66 | DELTA 1-PYRROLINE-5-CARBOXYLATE |
| | | | | | | | SYNTHETASE (P5CS) [INCLUDES: |
| | | | | | | | GLUTAMATE 5-KINASE (GAMMA- |
| | | | | | | | GLUTAMYL KINASE) (GK); GAMMA- |
| | | | | | | | GLUTAMYL PHOSPHATE REDUCTASE (GPR) |
| | | | | | | | (GLUTAMATE-5-SEMIALDEHYDE |
| | | | | | | | DEHYDROGENASE) (GLUTAMYL-GAMMA- |
| | | | | | | | SEMIALDEHYDE DEHYDROGENASE)] |
| | | | | | | | >gi\|7488983\|pir\|\|T |
| 1308 | LIB3602-030-Q6-K1-B9 | 6225817 | BLASTX | 500 | 2.00E−50 | 65 | DELTA 1-PYRROLINE-5-CARBOXYLATE |
| | | | | | | | SYNTHETASE (P5CS) [INCLUDES: |
| | | | | | | | GLUTAMATE 5-KINASE (GAMMA- |
| | | | | | | | GLUTAMYL KINASE) (GK); GAMMA- |
| | | | | | | | GLUTAMYL PHOSPHATE REDUCTASE (GPR) |
| | | | | | | | (GLUTAMATE-5-SEMIALDEHYDE |
| | | | | | | | DEHYDROGENASE) (GLUTAMYL-GAMMA- |
| | | | | | | | SEMIALDEHYDE DEHYDROGENASE)] |
| | | | | | | | >gi\|7488983\|pir\|\|T |
| 1309 | LIB3602-101-Q1-K1-E10 | 6225817 | BLASTX | 351 | 4.00E−37 | 77 | DELTA 1-PYRROLINE-5-CARBOXYLATE |
| | | | | | | | SYNTHETASE (P5CS) [INCLUDES: |
| | | | | | | | GLUTAMATE 5-KINASE (GAMMA- |
| | | | | | | | GLUTAMYL KINASE) (GK); GAMMA- |
| | | | | | | | GLUTAMYL PHOSPHATE REDUCTASE (GPR) |
| | | | | | | | (GLUTAMATE-5-SEMIALDEHYDE |
| | | | | | | | DEHYDROGENASE) (GLUTAMYL-GAMMA- |
| | | | | | | | SEMIALDEHYDE DEHYDROGENASE)] |
| | | | | | | | >gi\|7488983\|pir\|\|T |
| 1310 | LIB3602-048-Q6-K1QA-B4 | 6225817 | BLASTX | 289 | 1.00E−36 | 67 | DELTA 1-PYRROLINE-5-CARBOXYLATE |
| | | | | | | | SYNTHETASE (P5CS) [INCLUDES: |
| | | | | | | | GLUTAMATE 5-KINASE (GAMMA- |
| | | | | | | | GLUTAMYL KINASE) (GK); GAMMA- |
| | | | | | | | GLUTAMYL PHOSPHATE REDUCTASE (GPR) |
| | | | | | | | (GLUTAMATE-5-SEMIALDEHYDE |
| | | | | | | | DEHYDROGENASE) (GLUTAMYL-GAMMA- |
| | | | | | | | SEMIALDEHYDE DEHYDROGENASE)] |
| | | | | | | | >gi\|7488983\|pir\|\|T |
| 1311 | LIB3602-087-Q6-K1-D12 | 6225817 | BLASTX | 348 | 1.00E−33 | 71 | DELTA 1-PYRROLINE-5-CARBOXYLATE |
| | | | | | | | SYNTHETASE (P5CS) [INCLUDES: |
| | | | | | | | GLUTAMATE 5-KINASE (GAMMA- |
| | | | | | | | GLUTAMYL KINASE) (GK); GAMMA- |
| | | | | | | | GLUTAMYL PHOSPHATE REDUCTASE (GPR) |
| | | | | | | | (GLUTAMATE-5-SEMIALDEHYDE |
| | | | | | | | DEHYDROGENASE) (GLUTAMYL-GAMMA- |
| | | | | | | | SEMIALDEHYDE DEHYDROGENASE)] |
| | | | | | | | >gi\|7488983\|pir\|\|T |
| 1312 | LIB3602-058-Q6-K1-C6 | 7019398 | BLASTN | 37 | 5.00E−11 | 100 | Homo Sapiens gonadotropin-regulated testicular |
| | | | | | | | RNA helicase (GRTH), mRNA |
| 1313 | LIB3602-077-Q6-K6-C7 | 7019398 | BLASTN | 36 | 7.00E−11 | 100 | Homo Sapiens gonadotropin-regulated testicular |
| | | | | | | | RNA helicase (GRTH), mRNA |
| 1314 | LIB3602-040-Q6-K1-A11 | 7019398 | BLASTN | 36 | 2.00E−10 | 100 | Homo Sapiens gonadotropin-regulated testicular |
| | | | | | | | RNA helicase (GRTH), mRNA |
| 1315 | LIB3602-079-Q6-K6-A4 | 7019398 | BLASTN | 36 | 2.00E−10 | 100 | Homo Sapiens gonadotropin-regulated testicular |
| | | | | | | | RNA helicase (GRTH), mRNA |
| 1316 | LIB3602-118-Q1-K1-B10 | 7019398 | BLASTN | 36 | 2.00E−10 | 100 | Homo Sapiens gonadotropin-regulated testicular |
| | | | | | | | RNA helicase (GRTH), mRNA |
| 1317 | LIB3602-069-Q1-K1-F2 | 4581162 | BLASTX | 499 | 3.00E−50 | 78 | (AC006220) putative microtubule-associated |
| | | | | | | | protein [Arabidopsis thaliana] |
| 1318 | LIB3602-068-Q1-K1-F2 | 4581162 | BLASTX | 499 | 3.00E−50 | 78 | (AC006220) putative microtubule-associated |
| | | | | | | | protein [Arabidopsis thaliana] |
| 1319 | LIB3602-039-Q6-K1-E5 | 4581162 | BLASTX | 499 | 3.00E−50 | 78 | (AC006220) putative microtubule-associated |
| | | | | | | | protein [Arabidopsis thaliana] |
| 1320 | LIB3602-047-Q6-K1-F7 | 4581162 | BLASTX | 476 | 1.00E−47 | 77 | (AC006220) putative microtubule-associated |
| | | | | | | | protein [Arabidopsis thaliana] |
| 1321 | LIB3602-086-Q6-K1-F1 | 4581162 | BLASTX | 353 | 3.00E−33 | 58 | (AC006220) putative microtubule-associated |
| | | | | | | | protein [Arabidopsis thaliana] |
| 1322 | LIB3602-045-Q6-K1-B9 | 968987 | BLASTX | 232 | 4.00E−19 | 52 | (U31648) ferritin [Glycine max] |
| 1323 | LIB3602-102-Q1-K1-A4 | 968987 | BLASTX | 229 | 1.00E−18 | 51 | (U31648) ferritin [Glycine max] |
| 1324 | LIB3602-105-Q1-K1-G6 | 968987 | BLASTX | 218 | 2.00E−17 | 54 | (U31648) ferritin [Glycine max] |
| 1325 | LIB3602-067-Q1-K1-A11 | 968987 | BLASTX | 218 | 2.00E−17 | 54 | (U31648) ferritin [Glycine max] |
| 1326 | LIB3602-070-Q1-K1-H9 | 968987 | BLASTX | 201 | 2.00E−15 | 52 | (U31648) ferritin [Glycine max] |
| 1327 | LIB3602-095-Q6-K6-H11 | 4966342 | BLASTX | 224 | 5.00E−18 | 46 | (AC006341) ESTs gb\|T04357 and gb\|AA595092 |
| | | | | | | | come from this gene. [Arabidopsis thaliana] |
| 1328 | LIB3602-079-Q6-K6-C9 | 4966342 | BLASTX | 224 | 5.00E−18 | 46 | (AC006341) ESTs gb\|T04357 and gb\|AA595092 |
| | | | | | | | come from this gene. [Arabidopsis thaliana] |
| 1329 | LIB3602-116-Q1-K1-H11 | 4966342 | BLASTX | 224 | 6.00E−18 | 46 | (AC006341) ESTs gb\|T04357 and gb\|AA595092 |
| | | | | | | | come from this gene. [Arabidopsis thaliana] |
| 1330 | LIB3602-112-Q1-K1-H11 | 4966342 | BLASTX | 207 | 5.00E−16 | 46 | (AC006341) ESTs gb\|T04357 and gb\|AA595092 |
| | | | | | | | come from this gene. [Arabidopsis thaliana] |
| 1331 | LIB3602-089-Q6-K6-H11 | 4966342 | BLASTX | 203 | 1.00E−15 | 46 | (AC006341) ESTs gb\|T04357 and gb\|AA595092 |
| | | | | | | | come from this gene. [Arabidopsis thaliana] |
| 1332 | LIB3602-066-Q1-K6-C9 | 6714454 | BLASTX | 336 | 4.00E−31 | 61 | (AC011620) putative 60S ribosomal protein L22 |
| | | | | | | | [Arabidopsis thaliana] |
| 1333 | LIB3602-051-Q6-K1-D10 | 6714454 | BLASTX | 276 | 3.00E−24 | 61 | (AC011620) putative 60S ribosomal protein L22 |
| | | | | | | | [Arabidopsis thaliana] |
| 1334 | LIB3602-008-Q6-K1-H9 | 6714454 | BLASTX | 255 | 8.00E−22 | 60 | (AC011620) putative 60S ribosomal protein L22 |
| | | | | | | | [Arabidopsis thaliana] |
| 1335 | LIB3602-103-Q1-K1-F5 | 6714454 | BLASTX | 158 | 8.00E−14 | 54 | (AC011620) putative 60S ribosomal protein L22 |
| | | | | | | | [Arabidopsis thaliana] |
| 1336 | LIB3602-018-Q6-K1-A9 | 6714454 | BLASTX | 163 | 5.00E−11 | 57 | (AC011620) putative 60S ribosomal protein L22 |
| | | | | | | | [Arabidopsis thaliana] |
| 1337 | LIB3602-066-Q1-K6-H11 | 1805719 | BLASTX | 497 | 4.00E−60 | 70 | (Y10657) Rubisco activase [Chlorococcum littorale] |
| 1338 | LIB3602-106-Q1-K1-E4 | 1805719 | BLASTX | 497 | 4.00E−60 | 70 | (Y10657) Rubisco activase [Chlorococcum littorale] |
| 1339 | LIB3602-029-Q6-K1-A5 | 1805719 | BLASTX | 558 | 3.00E−57 | 71 | (Y10657) Rubisco activase [Chlorococcum littorale] |
| 1340 | LIB3602-040-Q6-K1-A10 | 1805719 | BLASTX | 554 | 6.00E−57 | 81 | (Y10657) Rubisco activase [Chlorococcum littorale] |
| 1341 | LIB3602-041-Q6-K1-H5 | 1805719 | BLASTX | 361 | 3.00E−34 | 64 | (Y10657) Rubisco activase [Chlorococcum littorale] |
| 1342 | LIB3602-048-Q6-K1QA-F3 | 595768 | BLASTX | 157 | 2.00E−10 | 66 | (U13866) non-functional lacZ alpha peptide |
| | | | | | | | [unidentified cloning vector] |
| 1343 | LIB3602-016-Q6-K1-E12 | 595768 | BLASTX | 157 | 3.00E−10 | 65 | (U13866) non-functional lacZ alpha peptide |
| | | | | | | | [unidentified cloning vector] |
| 1344 | LIB3602-056-Q6-K1-B5 | 595768 | BLASTX | 151 | 1.00E−09 | 63 | (U13866) non-functional lacZ alpha peptide |
| | | | | | | | [unidentified cloning vector] |
| 1345 | LIB3602-088-Q6-K6-A11 | 595768 | BLASTX | 148 | 4.00E−09 | 56 | (U13866) non-functional lacZ alpha peptide |
| | | | | | | | [unidentified cloning vector] |
| 1346 | LIB3602-017-Q6-K1-F3 | 7019661 | BLASTX | 318 | 3.00E−29 | 86 | (AL132954) ribosomal L23a-like protein |
| | | | | | | | [Arabidopsis thaliana] |
| 1347 | LIB3602-115-Q1-K1-G4 | 7019661 | BLASTX | 203 | 9.00E−16 | 61 | (AL132954) ribosomal L23a-like protein |
| | | | | | | | [Arabidopsis thaliana] |
| 1348 | LIB3602-107-Q1-K1-E9 | 7019661 | BLASTX | 172 | 4.00E−12 | 75 | (AL132954) ribosomal L23a-like protein |
| | | | | | | | [Arabidopsis thaliana] |
| 1349 | LIB3602-067-Q1-K1-D9 | 7019661 | BLASTX | 149 | 2.00E−10 | 90 | (AL132954) ribosomal L23a-like protein |
| | | | | | | | [Arabidopsis thaliana] |
| 1350 | LIB3602-073-Q1-K1-A7 | 5706704 | BLASTX | 341 | 8.00E−32 | 64 | (AF112440) ribosomal protein S26 [Pisum sativum] |
| 1351 | LIB3602-111-Q1-K1-D1 | 5706704 | BLASTX | 236 | 2.00E−19 | 64 | (AF112440) ribosomal protein S26 [Pisum sativum] |
| 1352 | LIB3602-044-Q6-K1-B5 | 5706704 | BLASTX | 231 | 5.00E−19 | 63 | (AF112440) ribosomal protein S26 [Pisum sativum] |
| 1353 | LIB3602-053-Q6-K1-A7 | 5706704 | BLASTX | 152 | 2.00E−10 | 55 | (AF112440) ribosomal protein S26 [Pisum sativum] |
| 1354 | LIB3602-059-Q6-K1-C4 | 399014 | BLASTX | 685 | 4.00E−72 | 74 | ADP, ATP CARRIER PROTEIN (ADP/ATP |
| | | | | | | | TRANSLOCASE) (ADENINE NUCLEOTIDE |
| | | | | | | | TRANSLOCATOR) (ANT) >gi\|99444\|pir\|\|A41677 |
| | | | | | | | ADP, ATP carrier protein - Chlorella kessleri |
| | | | | | | | >gi\|516597\|gb\|AAA33027.1\| (M76669) ATP/ADP |
| | | | | | | | translocator [Chlorella kessleri] |
| 1355 | LIB3602-050-Q6-K1-F7 | 399014 | BLASTX | 656 | 8.00E−69 | 81 | ADP, ATP CARRIER PROTEIN (ADP/ATP |
| | | | | | | | TRANSLOCASE) (ADENINE NUCLEOTIDE |
| | | | | | | | TRANSLOCATOR) (ANT) >gi\|99444\|pir\|\|A41677 |
| | | | | | | | ADP, ATP carrier protein - Chlorella kessleri |
| | | | | | | | >gi\|516597\|gb\|AAA33027.1\| (M76669) ATP/ADP |
| | | | | | | | translocator [Chlorella kessleri] |
| 1356 | LIB3602-018-Q6-K1-E11 | 399014 | BLASTX | 379 | 3.00E−36 | 72 | ADP, ATP CARRIER PROTEIN (ADP/ATP |
| | | | | | | | TRANSLOCASE) (ADENINE NUCLEOTIDE |
| | | | | | | | TRANSLOCATOR) (ANT) >gi\|99444\|pir\|\|A41677 |
| | | | | | | | ADP, ATP carrier protein - Chlorella kessleri |
| | | | | | | | >gi\|516597\|gb\|AAA33027.1\| (M76669) ATP/ADP |
| | | | | | | | translocator [Chlorella kessleri] |
| 1357 | LIB3602-044-Q6-K1-E11 | 399014 | BLASTX | 176 | 1.00E−12 | 81 | ADP, ATP CARRIER PROTEIN (ADP/ATP |
| | | | | | | | TRANSLOCASE) (ADENINE NUCLEOTIDE |
| | | | | | | | TRANSLOCATOR) (ANT) >gi\|99444\|pir\|\|A41677 |
| | | | | | | | ADP, ATP carrier protein - Chlorella kessleri |
| | | | | | | | >gi\|516597\|gb\|AAA33027.1\| (M76669) ATP/ADP |
| | | | | | | | translocator [Chlorella kessleri] |
| 1358 | LIB3602-067-Q1-K1-E2 | 7294504 | BLASTX | 338 | 1.00E−31 | 63 | (AE003539) RpS4 gene product [alt 2] [Drosophila |
| | | | | | | | melanogaster] >gi\|7294505\|gb\|AAF49847.1\| |
| | | | | | | | (AE003539) RpS4 gene product [alt 1] [Drosophila |
| | | | | | | | melanogaster] |
| 1359 | LIB3602-051-Q6-K1-A7 | 7294504 | BLASTX | 272 | 5.00E−24 | 69 | (AE003539) RpS4 gene product [alt 2] [Drosophila |
| | | | | | | | melanogaster] >gi\|7294505\|gb\|AAF49847.1\| |
| | | | | | | | (AE003539) RpS4 gene product [alt 1] [Drosophila |
| | | | | | | | melanogaster] |
| 1360 | LIB3602-003-Q1-K1-E9 | 7294504 | BLASTX | 261 | 9.00E−23 | 67 | (AE003539) RpS4 gene product [alt 2] [Drosophila |
| | | | | | | | melanogaster] >gi\|7294505\|gb\|AAF49847.1\| |
| | | | | | | | (AE003539) RpS4 gene product [alt 1] [Drosophila |
| | | | | | | | melanogaster] |
| 1361 | LIB3602-006-Q1-K1-E3 | 7294504 | BLASTX | 214 | 7.00E−21 | 68 | (AE003539) RpS4 gene product [alt 2] [Drosophila |
| | | | | | | | melanogaster] >gi\|7294505\|gb\|AAF49847.1\| |
| | | | | | | | (AE003539) RpS4 gene product [alt 1] [Drosophila |
| | | | | | | | melanogaster] |
| 1362 | LIB3602-054-Q6-K1-C12 | 7440943 | BLASTX | 498 | 3.00E−50 | 72 | ribosomal protein L19.T2H3.3 - Arabidopsis |
| | | | | | | | thaliana >gi\|3377797\|gb\|AAC28170.1\| (AF075597) |
| | | | | | | | Similar to 60S ribosome protein L19; coded for by |
| | | | | | | | A. thaliana cDNA T04719; coded for by A. thaliana |
| | | | | | | | cDNA H36046; coded for by A. thaliana cDNA |
| | | | | | | | T44067; coded for by A |
| 1363 | LIB3602-043-Q6-K1-E10 | 7440943 | BLASTX | 341 | 6.00E−32 | 68 | ribosomal protein L19.T2H3.3 - Arabidopsis |
| | | | | | | | thaliana >gi\|3377797\|gb\|AAC28170.1\| (AF075597) |
| | | | | | | | Similar to 60S ribosome protein L19; coded for by |
| | | | | | | | A. thaliana cDNA T04719; coded for by A. thaliana |
| | | | | | | | cDNA H36046; coded for by A. thaliana cDNA |
| | | | | | | | T44067; coded for by A |
| 1364 | LIB3602-044-Q6-K1-H8 | 7440943 | BLASTX | 251 | 2.00E−21 | 63 | ribosomal protein L19.T2H3.3 - Arabidopsis |
| | | | | | | | thaliana >gi\|3377797\|gb\|AAC28170.1\| (AF075597) |
| | | | | | | | Similar to 60S ribosome protein L19; coded for by |
| | | | | | | | A. thaliana cDNA T04719; coded for by A. thaliana |
| | | | | | | | cDNA H36046; coded for by A. thaliana cDNA |
| | | | | | | | T44067; coded for by A |
| 1365 | LIB3602-039-Q6-K1-B8 | 7440943 | BLASTX | 190 | 3.00E−14 | 75 | ribosomal protein L19.T2H3.3 - Arabidopsis |
| | | | | | | | thaliana >gi\|3377797\|gb\|AAC28170.1\| (AF075597) |
| | | | | | | | Similar to 60S ribosome protein L19; coded for by |
| | | | | | | | A. thaliana cDNA T04719; coded for by A. thaliana |
| | | | | | | | cDNA H36046; coded for by A. thaliana cDNA |
| | | | | | | | T44067; coded for by A |
| 1366 | LIB3602-022-Q6-K1-E8 | 7433282 | BLASTX | 575 | 2.00E−59 | 74 | omega 6 desaturase (EC 1.14.99.-) precursor, |
| | | | | | | | chloroplast - Chlamydomonas reinhardtii |
| | | | | | | | >gi\|2696717\|dbj\|BAA23881.1\| (AB007640) |
| | | | | | | | chloroplast w6 desaturase [Chlamydomonas |
| | | | | | | | reinhardtii] |
| 1367 | LIB3602-012-Q6-K1-F3 | 7433282 | BLASTX | 394 | 4.00E−38 | 68 | omega 6 desaturase (EC 1.14.99.-) precursor, |
| | | | | | | | chloroplast - Chlamydomonas reinhardtii |
| | | | | | | | >gi\|2696717\|dbj\|BAA23881.1\| (AB007640) |
| | | | | | | | chloroplast w6 desaturase [Chlamydomonas |
| | | | | | | | reinhardtii] |
| 1368 | LIB3602-016-Q6-K1-D3 | 7433282 | BLASTX | 151 | 7.00E−10 | 59 | omega 6 desaturase (EC 1.14.99.-) precursor, |
| | | | | | | | chloroplast - Chlamydomonas reinhardtii |
| | | | | | | | >gi\|2696717\|dbj\|BAA23881.1\| (AB007640) |
| | | | | | | | chloroplast w6 desaturase [Chlamydomonas |
| | | | | | | | reinhardtii] |
| 1369 | LIB3602-053-Q6-K1-B7 | 7433282 | BLASTX | 151 | 1.00E−09 | 59 | omega 6 desaturase (EC 1.14.99.-) precursor, |
| | | | | | | | chloroplast - Chlamydomonas reinhardtii |
| | | | | | | | >gi\|2696717\|dbj\|BAA23881.1\| (AB007640) |
| | | | | | | | chloroplast w6 desaturase [Chlamydomonas |
| | | | | | | | reinhardtii] |
| 1370 | LIB3602-083-Q6-K6-C6 | 7484374 | BLASTX | 433 | 2.00E−42 | 51 | envelope protein LIP-36G1, low CO2 inducible - |
| | | | | | | | Chlamydomonas reinhardtii |
| | | | | | | | >gi\|2459573\|gb\|AAB71743.1\| (U75345) envelope |
| | | | | | | | protein [Chlamydomonas reinhardtii] |
| 1371 | LIB3602-118-Q1-K1-D1 | 7484374 | BLASTX | 334 | 4.00E−40 | 68 | envelope protein LIP-36G1, low CO2 inducible - |
| | | | | | | | Chlamydomonas reinhardtii |
| | | | | | | | >gi\|2459573\|gb\|AAB71743.1\| (U75345) envelope |
| | | | | | | | protein [Chlamydomonas reinhardtii] |
| 1372 | LIB3602-109-Q1-K1-C6 | 7484374 | BLASTX | 393 | 7.00E−38 | 50 | envelope protein LIP-36G1, low CO2 inducible - |
| | | | | | | | Chlamydomonas reinhardtii |
| | | | | | | | >gi\|2459573\|gb\|AAB71743.1\| (U75345) envelope |
| | | | | | | | protein [Chlamydomonas reinhardtii] |
| 1373 | LIB3602-064-Q1-K6-F7 | 7484374 | BLASTX | 153 | 6.00E−10 | 42 | envelope protein LIP-36G1, low CO2 inducible - |
| | | | | | | | Chlamydomonas reinhardtii |
| | | | | | | | >gi\|2459573\|gb\|AAB71743.1\| (U75345) envelope |
| | | | | | | | protein [Chlamydomonas reinhardtii] |
| 1374 | LIB3602-112-Q1-K1-G4 | 3776572 | BLASTX | 307 | 9.00E−28 | 38 | (AC005388) ESTs gb\|R65052, gb\|AA712146, |
| | | | | | | | gb\|H76533, gb\|H76282, gb\|AA650771, gb\|H76287, |
| | | | | | | | gb\|AA650887, gb\|N37383, gb\|Z29721 and |
| | | | | | | | gb\|Z29722 come from this gene. [Arabidopsis |
| | | | | | | | thaliana] |
| 1375 | LIB3602-032-Q6-K1-G3 | 3776572 | BLASTX | 188 | 6.00E−14 | 38 | (AC005388) ESTs gb\|R65052, gb\|AA712146, |
| | | | | | | | gb\|H76533, gb\|H76282, gb\|AA650771, gb\|H76287, |
| | | | | | | | gb\|AA650887, gb\|N37383, gb\|Z29721 and |
| | | | | | | | gb\|Z29722 come from this gene. [Arabidopsis |
| | | | | | | | thaliana] |
| 1376 | LIB3602-032-Q6-K1-F12 | 3776572 | BLASTX | 187 | 8.00E−14 | 39 | (AC005388) ESTs gb\|R65052, gb\|AA712146, |
| | | | | | | | gb\|H76533, gb\|H76282, gb\|AA650771, gb\|H76287, |
| | | | | | | | gb\|AA650887, gb\|N37383, gb\|Z29721 and |
| | | | | | | | gb\|Z29722 come from this gene. [Arabidopsis |
| | | | | | | | thaliana] |
| 1377 | LIB3602-044-Q6-K1-E4 | 3776572 | BLASTX | 185 | 1.00E−13 | 39 | (AC005388) ESTs gb\|R65052, gb\|AA712146, |
| | | | | | | | gb\|H76533, gb\|H76282, gb\|AA650771, gb\|H76287, |
| | | | | | | | gb\|AA650887, gb\|N37383, gb\|Z29721 and |
| | | | | | | | gb\|Z29722 come from this gene. [Arabidopsis |
| | | | | | | | thaliana] |
| 1378 | LIB3602-027-Q6-K1-C9 | 2492870 | BLASTX | 474 | 2.00E−47 | 52 | SERINE--GLYOXYLATE |
| | | | | | | | AMINOTRANSFERASE (SGAT) |
| 1379 | LIB3602-046-Q6-K1-F9 | 2492870 | BLASTX | 422 | 2.00E−41 | 51 | SERINE--GLYOXYLATE |
| | | | | | | | AMINOTRANSFERASE (SGAT) |
| 1380 | LIB3602-013-Q6-K1-B5 | 2492870 | BLASTX | 419 | 5.00E−41 | 51 | SERINE--GLYOXYLATE |
| | | | | | | | AMINOTRANSFERASE (SGAT) |
| 1381 | LIB3602-045-Q6-K1-D2 | 2492870 | BLASTX | 259 | 3.00E−22 | 50 | SERINE--GLYOXYLATE |
| | | | | | | | AMINOTRANSFERASE (SGAT) |
| 1382 | LIB3602-076-Q6-K6-G4 | 6513924 | BLASTX | 182 | 3.00E−20 | 42 | (AC011664) putative 40S ribosomal protein S19 |
| | | | | | | | [Arabidopsis thaliana] |
| 1383 | LIB3602-085-Q6-K1-E2 | 6513924 | BLASTX | 200 | 3.00E−15 | 35 | (AC011664) putative 40S ribosomal protein S19 |
| | | | | | | | [Arabidopsis thaliana] |
| 1384 | LIB3602-077-Q6-K6-G2 | 6513924 | BLASTX | 146 | 4.00E−09 | 55 | (AC011664) putative 40S ribosomal protein S19 |
| | | | | | | | [Arabidopsis thaliana] |
| 1385 | LIB3602-065-Q1-K6-C7 | 6513924 | BLASTX | 146 | 4.00E−09 | 55 | (AC011664) putative 40S ribosomal protein S19 |
| | | | | | | | [Arabidopsis thaliana] |
| 1386 | LIB3602-113-Q1-K1-A2 | 4406134 | BLASTX | 320 | 2.00E−29 | 62 | (AF083392) promoter active fragment E3 |
| | | | | | | | [Synechococcus PCC7942] |
| 1387 | LIB3602-112-Q1-K1-A2 | 4406134 | BLASTX | 320 | 3.00E−29 | 62 | (AF083392) promoter active fragment E3 |
| | | | | | | | [Synechococcus PCC7942] |
| 1388 | LIB3602-093-Q6-K6-A6 | 4406134 | BLASTX | 320 | 3.00E−29 | 62 | (AF083392) promoter active fragment E3 |
| | | | | | | | [Synechococcus PCC7942] |
| 1389 | LIB3602-044-Q6-K1-B9 | 4406134 | BLASTX | 272 | 9.00E−24 | 66 | (AF083392) promoter active fragment E3 |
| | | | | | | | [Synechococcus PCC7942] |
| 1390 | LIB3602-007-Q1-K1-F9 | 7448603 | BLASTX | 650 | 7.00E−68 | 53 | flavoprotein, 63.5K - Synechocystis sp. (strain PCC |
| | | | | | | | 6803) >gi\|1001242\|dbj\|BAA10483.1\| (D64003) |
| | | | | | | | flavoprotein [Synechocystis sp.] |
| 1391 | LIB3602-072-Q1-K1-G1 | 7448603 | BLASTX | 504 | 8.00E−51 | 59 | flavoprotein, 63.5K - Synechocystis sp. (strain PCC |
| | | | | | | | 6803) >gi\|1001242\|dbj\|BAA10483.1\| (D64003) |
| | | | | | | | flavoprotein [Synechocystis sp.] |
| 1392 | LIB3602-004-Q1-K1-G7 | 7448603 | BLASTX | 226 | 2.00E−18 | 57 | flavoprotein, 63.5K - Synechocystis sp. (strain PCC |
| | | | | | | | 6803) >gi\|1001242\|dbj\|BAA10483.1\| (D64003) |
| | | | | | | | flavoprotein [Synechocystis sp.] |
| 1393 | LIB3602-107-Q1-K1-B1 | 7448603 | BLASTX | 141 | 2.00E−14 | 65 | flavoprotein, 63.5K - Synechocystis sp. (strain PCC |
| | | | | | | | 6803) >gi\|1001242\|dbj\|BAA10483.1\| (D64003) |
| | | | | | | | flavoprotein [Synechocystis sp.] |
| 1394 | LIB3602-090-Q6-K6-B12 | 2290991 | BLASTX | 425 | 1.00E−41 | 57 | (AF006000) unknown [Bordetella pertussis] |
| 1395 | LIB3602-091-Q6-K6-B12 | 2290991 | BLASTX | 425 | 1.00E−41 | 57 | (AF006000) unknown [Bordetella pertussis] |
| 1396 | LIB3602-094-Q6-K6-B12 | 2290991 | BLASTX | 347 | 6.00E−34 | 55 | (AF006000) unknown [Bordetella pertussis] |
| 1397 | LIB3602-039-Q6-K1-D1 | 2290991 | BLASTX | 297 | 1.00E−26 | 55 | (AF006000) unknown [Bordetella pertussis] |
| 1398 | LIB3602-074-Q1-K1-A5 | 6714451 | BLASTX | 319 | 1.00E−37 | 80 | (AC011620) putative 60S ribosomal protein L18 |
| | | | | | | | [Arabidopsis thaliana] |
| 1399 | LIB3602-037-Q6-K1-F9 | 6714451 | BLASTX | 370 | 2.00E−35 | 78 | (AC011620) putative 60S ribosomal protein L18 |
| | | | | | | | [Arabidopsis thaliana] |
| 1400 | LIB3602-069-Q1-K1-D4 | 6714451 | BLASTX | 308 | 2.00E−28 | 81 | (AC011620) putative 60S ribosomal protein L18 |
| | | | | | | | [Arabidopsis thaliana] |
| 1401 | LIB3602-086-Q6-K1-B2 | 6714451 | BLASTX | 163 | 3.00E−11 | 85 | (AC011620) putative 60S ribosomal protein L18 |
| | | | | | | | [Arabidopsis thaliana] |
| 1402 | LIB3602-056-Q6-K1-B11 | 3851636 | BLASTX | 482 | 2.00E−48 | 60 | (AF098519) unknown [Avicennia marina] |
| | | | | | | | >gi\|4128206\|gb\|AAD03501.1\| (AF056316) 40S |
| | | | | | | | ribosome protein S7 [Avicennia marina] |
| 1403 | LIB3602-047-Q6-K1-D3 | 3851636 | BLASTX | 300 | 3.00E−27 | 67 | (AF098519) unknown [Avicennia marina] |
| | | | | | | | >gi\|4128206\|gb\|AAD03501.1\| (AF056316) 40S |
| | | | | | | | ribosome protein S7 [Avicennia marina] |
| 1404 | LIB3602-084-Q6-K1-A1 | 3851636 | BLASTX | 192 | 2.00E−14 | 40 | (AF098519) unknown [Avicennia marina] |
| | | | | | | | >gi\|4128206\|gb\|AAD03501.1\| (AF056316) 40S |
| | | | | | | | ribosome protein S7 [Avicennia marina] |
| 1405 | LIB3602-091-Q6-K6-B3 | 3851636 | BLASTX | 146 | 3.00E−14 | 62 | (AF098519) unknown [Avicennia marina] |
| | | | | | | | >gi\|4128206\|gb\|AAD03501.1\| (AF056316) 40S |
| | | | | | | | ribosome protein S7 [Avicennia marina] |
| 1406 | LIB3602-071-Q1-K1-D7 | 4836876 | BLASTX | 565 | 5.00E−58 | 66 | (AC007260) Similar to dTDP-D-glucose 4,6- |
| | | | | | | | dehydratase [Arabidopsis thaliana] |
| 1407 | LIB3602-010-Q6-K1-B3 | 4836876 | BLASTX | 441 | 2.00E−43 | 57 | (AC007260) Similar to dTDP-D-glucose 4,6- |
| | | | | | | | dehydratase [Arabidopsis thaliana] |
| 1408 | LIB3602-037-Q6-K1-F10 | 4836876 | BLASTX | 334 | 6.00E−31 | 52 | (AC007260) Similar to dTDP-D-glucose 4,6- |
| | | | | | | | dehydratase [Arabidopsis thaliana] |
| 1409 | LIB3602-057-Q6-K1-A3 | 4836876 | BLASTX | 170 | 2.00E−24 | 52 | (AC007260) Similar to dTDP-D-glucose 4,6- |
| | | | | | | | dehydratase [Arabidopsis thaliana] |
| 1410 | LIB3602-062-Q6-K1-D10 | 5902590 | BLASTX | 577 | 2.00E−59 | 69 | (AF110783) rieske iron-sulfur protein precursor |
| | | | | | | | [Volvox carteri f. nagariensis] |
| 1411 | LIB3602-013-Q6-K1-D4 | 5902590 | BLASTX | 540 | 3.00E−55 | 77 | (AF110783) rieske iron-sulfur protein precursor |
| | | | | | | | [Volvox carteri f. nagariensis] |
| 1412 | LIB3602-008-Q6-K1-B11 | 5902590 | BLASTX | 458 | 2.00E−45 | 81 | (AF110783) rieske iron-sulfur protein precursor |
| | | | | | | | [Volvox carteri f. nagariensis] |
| 1413 | LIB3602-008-Q6-K1-H6 | 5902590 | BLASTX | 378 | 4.00E−36 | 57 | (AF110783) rieske iron-sulfur protein precursor |
| | | | | | | | [Volvox carteri f. nagariensis] |
| 1414 | LIB3602-050-Q6-K1-B2 | 7488227 | BLASTX | 566 | 3.00E−58 | 74 | prohibitin-like protein F20O9.200 - Arabidopsis |
| | | | | | | | thaliana >gi\|2842494\|emb\|CAA16891.1\| |
| | | | | | | | (AL021749) prohibitin-like protein [Arabidopsis |
| | | | | | | | thaliana] >gi\|4097688\|gb\|AAD00155.1\| (U66591) |
| | | | | | | | prohibitin 1 [Arabidopsis thaliana] |
| | | | | | | | >gi\|4097694\|gb\|AAD00158.1\| (U66594) proh |
| 1415 | LIB3602-011-Q6-K1-F1 | 7488227 | BLASTX | 483 | 2.00E−48 | 71 | prohibitin-like protein F20O9.200 - Arabidopsis |
| | | | | | | | thaliana >gi\|2842494\|emb\|CAA16891.1\| |
| | | | | | | | (AL021749) prohibitin-like protein [Arabidopsis |
| | | | | | | | thaliana] >gi\|4097688\|gb\|AAD00155.1\| (U66591) |
| | | | | | | | prohibitin 1 [Arabidopsis thaliana] |
| | | | | | | | >gi\|4097694\|gb\|AAD00158.1\| (U66594) proh |
| 1416 | LIB3602-086-Q6-K1-G3 | 7488227 | BLASTX | 451 | 1.00E−44 | 70 | prohibitin-like protein F20O9.200 - Arabidopsis |
| | | | | | | | thaliana >gi\|2842494\|emb\|CAA16891.1\| |
| | | | | | | | (AL021749) prohibitin-like protein [Arabidopsis |
| | | | | | | | thaliana] >gi\|4097688\|gb\|AAD00155.1\| (U66591) |
| | | | | | | | prohibitin 1 [Arabidopsis thaliana] |
| | | | | | | | >gi\|4097694\|gb\|AAD00158.1\| (U66594) proh |
| 1417 | LIB3602-013-Q6-K1-B6 | 7488227 | BLASTX | 398 | 2.00E−38 | 73 | prohibitin-like protein F20O9.200 - Arabidopsis |
| | | | | | | | thaliana >gi\|2842494\|emb\|CAA16891.1\| |
| | | | | | | | (AL021749) prohibitin-like protein [Arabidopsis |
| | | | | | | | thaliana] >gi\|4097688\|gb\|AAD00155.1\| (U66591) |
| | | | | | | | prohibitin 1 [Arabidopsis thaliana] |
| | | | | | | | >gi\|4097694\|gb\|AAD00158.1\| (U66594) proh |
| 1418 | LIB3602-005-Q1-K1-F3 | 7484402 | BLASTX | 398 | 2.00E−38 | 68 | protochlorophyllide reductase (EC 1.3.1.33) |
| | | | | | | | precursor - Chlamydomonas reinhardtii |
| | | | | | | | >gi\|1408176\|gb\|AAB04951.1\| (U36752) |
| | | | | | | | NADPH: protochlorophyllide oxidoreductase |
| | | | | | | | [Chlamydomonas reinhardtii] |
| 1419 | LIB3602-085-Q6-K1-C11 | 7484402 | BLASTX | 387 | 4.00E−37 | 66 | protochlorophyllide reductase (EC 1.3.1.33) |
| | | | | | | | precursor - Chlamydomonas reinhardtii |
| | | | | | | | >gi\|1408176\|gb\|AAB04951.1\| (U36752) |
| | | | | | | | NADPH: protochlorophyllide oxidoreductase |
| | | | | | | | [Chlamydomonas reinhardtii] |
| 1420 | LIB3602-112-Q1-K1-F1 | 7484402 | BLASTX | 222 | 6.00E−18 | 61 | protochlorophyllide reductase (EC 1.3.1.33) |
| | | | | | | | precursor - Chlamydomonas reinhardtii |
| | | | | | | | >gi\|1408176\|gb\|AAB04951.1\| (U36752) |
| | | | | | | | NADPH: protochlorophyllide oxidoreductase |
| | | | | | | | [Chlamydomonas reinhardtii] |
| 1421 | LIB3602-093-Q6-K6-C7 | 7484402 | BLASTX | 222 | 6.00E−18 | 61 | protochlorophyllide reductase (EC 1.3.1.33) |
| | | | | | | | precursor - Chlamydomonas reinhardtii |
| | | | | | | | >gi\|1408176\|gb\|AAB04951.1\| (U36752) |
| | | | | | | | NADPH: protochlorophyllide oxidoreductase |
| | | | | | | | [Chlamydomonas reinhardtii] |
| 1422 | LIB3602-050-Q6-K1-D11 | 7488810 | BLASTX | 276 | 3.00E−24 | 47 | hydroxyproline-rich glycoprotein 1 - garden pea |
| | | | | | | | (fragment) >gi\|1711036\|gb\|AAB97098.1\| (U78952) |
| | | | | | | | hydroxyproline rich glycoprotein PsHRGP1 [Pisum |
| | | | | | | | sativum] |
| 1423 | LIB3602-058-Q6-K1-B3 | 7488810 | BLASTX | 224 | 4.00E−18 | 40 | hydroxyproline-rich glycoprotein 1 - garden pea |
| | | | | | | | (fragment) >gi\|1711036\|gb\|AAB97098.1\| (U78952) |
| | | | | | | | hydroxyproline rich glycoprotein PsHRGP1 [Pisum |
| | | | | | | | sativum] |
| 1424 | LIB3602-055-Q6-K1-C6 | 7488810 | BLASTX | 224 | 4.00E−18 | 40 | hydroxyproline-rich glycoprotein 1 - garden pea |
| | | | | | | | (fragment) >gi\|1711036\|gb\|AAB97098.1\| (U78952) |
| | | | | | | | hydroxyproline rich glycoprotein PsHRGP1 [Pisum |
| | | | | | | | sativum] |
| 1425 | LIB3602-067-Q1-K1-E10 | 7488810 | BLASTX | 217 | 3.00E−17 | 39 | hydroxyproline-rich glycoprotein 1 - garden pea |
| | | | | | | | (fragment) >gi\|1711036\|gb\|AAB97098.1\| (U78952) |
| | | | | | | | hydroxyproline rich glycoprotein PsHRGP1 [Pisum |
| | | | | | | | sativum] |
| 1426 | LIB3602-091-Q6-K6-E10 | 3914425 | BLASTX | 696 | 2.00E−73 | 73 | PROTEASOME EPSILON CHAIN PRECURSOR |
| | | | | | | | (MACROPAIN EPSILON CHAIN) |
| | | | | | | | (MULTICATALYTIC ENDOPEPTIDASE |
| | | | | | | | COMPLEX EPSILON CHAIN) |
| | | | | | | | >gi\|2511596\|emb\|CAA74029.1\| (Y13695) |
| | | | | | | | multicatalytic endopeptidase complex, proteasome |
| | | | | | | | precursor, beta subunit [Arabidopsis thaliana] |
| | | | | | | | >gi\|3421 |
| 1427 | LIB3602-073-Q1-K1-E1 | 3914425 | BLASTX | 560 | 2.00E−57 | 71 | PROTEASOME EPSILON CHAIN PRECURSOR |
| | | | | | | | (MACROPAIN EPSILON CHAIN) |
| | | | | | | | (MULTICATALYTIC ENDOPEPTIDASE |
| | | | | | | | COMPLEX EPSILON CHAIN) |
| | | | | | | | >gi\|2511596\|emb\|CAA74029.1\| (Y13695) |
| | | | | | | | multicatalytic endopeptidase complex, proteasome |
| | | | | | | | precursor, beta subunit [Arabidopsis thaliana] |
| | | | | | | | >gi\|3421 |
| 1428 | LIB3602-102-Q1-K1-D3 | 3914425 | BLASTX | 560 | 2.00E−57 | 71 | PROTEASOME EPSILON CHAIN PRECURSOR |
| | | | | | | | (MACROPAIN EPSILON CHAIN) |
| | | | | | | | (MULTICATALYTIC ENDOPEPTIDASE |
| | | | | | | | COMPLEX EPSILON CHAIN) |
| | | | | | | | >gi\|2511596\|emb\|CAA74029.1\| (Y13695) |
| | | | | | | | multicatalytic endopeptidase complex, proteasome |
| | | | | | | | precursor, beta subunit [Arabidopsis thaliana] |
| | | | | | | | >gi\|3421 |
| 1429 | LIB3602-057-Q6-K1-D11 | 3914425 | BLASTX | 251 | 5.00E−37 | 69 | PROTEASOME EPSILON CHAIN PRECURSOR |
| | | | | | | | (MACROPAIN EPSILON CHAIN) |
| | | | | | | | (MULTICATALYTIC ENDOPEPTIDASE |
| | | | | | | | COMPLEX EPSILON CHAIN) |
| | | | | | | | >gi\|2511596\|emb\|CAA74029.1\| (Y13695) |
| | | | | | | | multicatalytic endopeptidase complex, proteasome |
| | | | | | | | precursor, beta subunit [Arabidopsis thaliana] |
| | | | | | | | >gi\|3421 |
| 1430 | LIB3602-050-Q6-K1-E4 | 131220 | BLASTX | 249 | 4.00E−21 | 48 | PHOTOSYSTEM I REACTION CENTRE |
| | | | | | | | SUBUNIT X PRECURSOR (LIGHT- |
| | | | | | | | HARVESTING COMPLEX I 8.4 KD PROTEIN) |
| | | | | | | | (PSI-K) (P37 PROTEIN) >gi\|81243\|pir\|\|S06684 |
| | | | | | | | photosystem I 8.4K protein precursor - |
| | | | | | | | Chlamydomonas reinhardtii >gi\|81249\|pir\|\|JQ0372 |
| | | | | | | | photosystem I protein P37 - Ch |
| 1431 | LIB3602-050-Q6-K1-E3 | 131220 | BLASTX | 248 | 6.00E−21 | 60 | PHOTOSYSTEM I REACTION CENTRE |
| | | | | | | | SUBUNIT X PRECURSOR (LIGHT- |
| | | | | | | | HARVESTING COMPLEX I 8.4 KD PROTEIN) |
| | | | | | | | (PSI-K) (P37 PROTEIN) >gi\|81243\|pir\|\|S06684 |
| | | | | | | | photosystem I 8.4K protein precursor - |
| | | | | | | | Chlamydomonas reinhardtii >gi\|81249\|pir\|\|JQ0372 |
| | | | | | | | photosystem I protein P37 - Ch |
| 1432 | LIB3602-011-Q6-K1-H6 | 131220 | BLASTX | 237 | 1.00E−19 | 58 | PHOTOSYSTEM I REACTION CENTRE |
| | | | | | | | SUBUNIT X PRECURSOR (LIGHT- |
| | | | | | | | HARVESTING COMPLEX I 8.4 KD PROTEIN) |
| | | | | | | | (PSI-K) (P37 PROTEIN) >gi\|81243\|pir\|\|S06684 |
| | | | | | | | photosystem I 8.4K protein precursor - |
| | | | | | | | Chlamydomonas reinhardtii >gi\|81249\|pir\|\|JQ0372 |
| | | | | | | | photosystem I protein P37 - Ch |
| 1433 | LIB3602-053-Q6-K1-G4 | 131220 | BLASTX | 202 | 8.00E−16 | 62 | PHOTOSYSTEM I REACTION CENTRE |
| | | | | | | | SUBUNIT X PRECURSOR (LIGHT- |
| | | | | | | | HARVESTING COMPLEX I 8.4 KD PROTEIN) |
| | | | | | | | (PSI-K) (P37 PROTEIN) >gi\|81243\|pir\|\|S06684 |
| | | | | | | | photosystem I 8.4K protein precursor - |
| | | | | | | | Chlamydomonas reinhardtii >gi\|81249\|pir\|\|JQ0372 |
| | | | | | | | photosystem I protein P37 - Ch |
| 1434 | LIB3602-003-Q1-K1-F1 | 7211992 | BLASTX | 537 | 7.00E−55 | 68 | (AC004809) Strong simialrity to the disulfide |
| | | | | | | | isomerase precursor homolog T21L14.14 |
| | | | | | | | gi\|2702281 from A. thaliana on BAC gb\|AC003033. |
| | | | | | | | [Arabidopsis thaliana] |
| 1435 | LIB3602-018-Q6-K1-G7 | 7211992 | BLASTX | 354 | 3.00E−33 | 48 | (AC004809) Strong simialrity to the disulfide |
| | | | | | | | isomerase precursor homolog T21L14.14 |
| | | | | | | | gi\|2702281 from A. thaliana on BAC gb\|AC003033. |
| | | | | | | | [Arabidopsis thaliana] |
| 1436 | LIB3602-063-Q1-K6-D12 | 7211992 | BLASTX | 344 | 4.00E−32 | 44 | (AC004809) Strong simialrity to the disulfide |
| | | | | | | | isomerase precursor homolog T21L14.14 |
| | | | | | | | gi\|2702281 from A. thaliana on BAC gb\|AC003033. |
| | | | | | | | [Arabidopsis thaliana] |
| 1437 | LIB3602-052-Q6-K1-C2 | 7211992 | BLASTX | 322 | 1.00E−29 | 48 | (AC004809) Strong simialrity to the disulfide |
| | | | | | | | isomerase precursor homolog T21L14.14 |
| | | | | | | | gi\|2702281 from A. thaliana on BAC gb\|AC003033. |
| | | | | | | | [Arabidopsis thaliana] |
| 1438 | LIB3602-115-Q1-K1-C12 | 2754849 | BLASTX | 565 | 5.00E−58 | 61 | (AF039000) putative serine-glyoxylate |
| | | | | | | | aminotransferase [Fritillaria agrestis] |
| 1439 | LIB3602-088-Q6-K6-C12 | 2754849 | BLASTX | 559 | 3.00E−57 | 59 | (AF039000) putative serine-glyoxylate |
| | | | | | | | aminotransferase [Fritillaria agrestis] |
| 1440 | LIB3602-104-Q1-K1-F7 | 2754849 | BLASTX | 557 | 5.00E−57 | 62 | (AF039000) putative serine-glyoxylate |
| | | | | | | | aminotransferase [Fritillaria agrestis] |
| 1441 | LIB3602-049-Q6-K1-G1 | 2754849 | BLASTX | 152 | 2.00E−12 | 39 | (AF039000) putative serine-glyoxylate |
| | | | | | | | aminotransferase [Fritillaria agrestis] |
| 1442 | LIB3602-035-Q1-K1-B2 | 2582351 | BLASTX | 473 | 3.00E−47 | 56 | (AF018639) unknown [Dictyostelium discoideum] |
| 1443 | LIB3602-034-Q6-K1-B2 | 2582351 | BLASTX | 456 | 3.00E−45 | 57 | (AF018639) unknown [Dictyostelium discoideum] |
| 1444 | LIB3602-008-Q6-K1-B1 | 2582351 | BLASTX | 277 | 2.00E−24 | 53 | (AF018639) unknown [Dictyostelium discoideum] |
| 1445 | LIB3602-008-Q6-K1-D5 | 2582351 | BLASTX | 212 | 9.00E−17 | 50 | (AF018639) unknown [Dictyostelium discoideum] |
| 1446 | LIB3602-113-Q1-K1-A1 | 3914473 | BLASTX | 456 | 2.00E−45 | 64 | PHOTOSYSTEM I REACTION CENTRE |
| | | | | | | | SUBUNIT XI PRECURSOR (SUBUNIT V) (PSI- |
| | | | | | | | L) >gi\|479684\|pir\|\|S35151 photosystem I chain XI |
| | | | | | | | precursor - spinach >gi\|396275\|emb\|CAA45775.1\| |
| | | | | | | | (X64445) subunit XI of photosystem I reaction |
| | | | | | | | center [Spinacia oleracea] |
| 1447 | LIB3602-115-Q1-K1-F8 | 3914473 | BLASTX | 404 | 4.00E−39 | 59 | PHOTOSYSTEM I REACTION CENTRE |
| | | | | | | | SUBUNIT XI PRECURSOR (SUBUNIT V) (PSI- |
| | | | | | | | L) >gi\|479684\|pir\|\|S35151 photosystem I chain XI |
| | | | | | | | precursor - spinach >gi\|396275\|emb\|CAA45775.1\| |
| | | | | | | | (X64445) subunit XI of photosystem I reaction |
| | | | | | | | center [Spinacia oleracea] |
| 1448 | LIB3602-106-Q1-K1-G12 | 3914473 | BLASTX | 372 | 2.00E−35 | 59 | PHOTOSYSTEM I REACTION CENTRE |
| | | | | | | | SUBUNIT XI PRECURSOR (SUBUNIT V) (PSI- |
| | | | | | | | L) >gi\|479684\|pir\|\|S35151 photosystem I chain XI |
| | | | | | | | precursor - spinach >gi\|396275\|emb\|CAA45775.1\| |
| | | | | | | | (X64445) subunit XI of photosystem I reaction |
| | | | | | | | center [Spinacia oleracea] |
| 1449 | LIB3602-019-Q6-K1-A5 | 3914473 | BLASTX | 324 | 5.00E−30 | 84 | PHOTOSYSTEM I REACTION CENTRE |
| | | | | | | | SUBUNIT XI PRECURSOR (SUBUNIT V) (PSI- |
| | | | | | | | L) >gi\|479684\|pir\|\|S35151 photosystem I chain XI |
| | | | | | | | precursor - spinach >gi\|396275\|emb\|CAA45775.1\| |
| | | | | | | | (X64445) subunit XI of photosystem I reaction |
| | | | | | | | center [Spinacia oleracea] |
| 1450 | LIB3602-034-Q6-K1-C8 | 1172816 | BLASTX | 647 | 1.00E−67 | 80 | 60S RIBOSOMAL PROTEIN L11A (L16A) |
| | | | | | | | >gi\|624938\|emb\|CAA57395.1\| (X81799) ribosomal |
| | | | | | | | protein L16 [Arabidopsis thaliana] |
| 1451 | LIB3602-079-Q6-K6-D10 | 1172816 | BLASTX | 613 | 1.00E−63 | 79 | 60S RIBOSOMAL PROTEIN L11A (L16A) |
| | | | | | | | >gi\|624938\|emb\|CAA57395.1\| (X81799) ribosomal |
| | | | | | | | protein L16 [Arabidopsis thaliana] |
| 1452 | LIB3602-077-Q6-K6-E5 | 1172816 | BLASTX | 613 | 1.00E−63 | 79 | 60S RIBOSOMAL PROTEIN L11A (L16A) |
| | | | | | | | >gi\|624938\|emb\|CAA57395.1\| (X81799) ribosomal |
| | | | | | | | protein L16 [Arabidopsis thaliana] |
| 1453 | LIB3602-035-Q1-K1-C8 | 1172816 | BLASTX | 613 | 1.00E−63 | 79 | 60S RIBOSOMAL PROTEIN L11A (L16A) |
| | | | | | | | >gi\|624938\|emb\|CAA57395.1\| (X81799) ribosomal |
| | | | | | | | protein L16 [Arabidopsis thaliana] |
| 1454 | LIB3602-035-Q1-K1-H8 | 3747050 | BLASTX | 435 | 9.00E−43 | 83 | (AF093540) ribosomal protein L26 [Zea mays] |
| 1455 | LIB3602-064-Q1-K6-C4 | 3747050 | BLASTX | 425 | 1.00E−41 | 83 | (AF093540) ribosomal protein L26 [Zea mays] |
| 1456 | LIB3602-036-Q6-K1-B6 | 3747050 | BLASTX | 256 | 1.00E−21 | 85 | (AF093540) ribosomal protein L26 [Zea mays] |
| 1457 | LIB3602-034-Q6-K1-H8 | 3747050 | BLASTX | 244 | 3.00E−21 | 81 | (AF093540) ribosomal protein L26 [Zea mays] |
| 1458 | LIB3602-112-Q1-K1-G6 | 118241 | BLASTX | 327 | 4.00E−30 | 49 | DIHYDRODIPICOLINATE SYNTHASE |
| | | | | | | | PRECURSOR (DHDPS) |
| 1459 | LIB3602-092-Q6-K6-G11 | 118241 | BLASTX | 327 | 4.00E−30 | 49 | DIHYDRODIPICOLINATE SYNTHASE |
| | | | | | | | PRECURSOR (DHDPS) |
| 1460 | LIB3602-029-Q6-K1-D5 | 118241 | BLASTX | 295 | 2.00E−26 | 55 | DIHYDRODIPICOLINATE SYNTHASE |
| | | | | | | | PRECURSOR (DHDPS) |
| 1461 | LIB3602-028-Q6-K1-C9 | 118241 | BLASTX | 279 | 1.00E−24 | 55 | DIHYDRODIPICOLINATE SYNTHASE |
| | | | | | | | PRECURSOR (DHDPS) |
| 1462 | LIB3602-076-Q6-K6-B11 | 7239379 | BLASTX | 647 | 6.00E−73 | 72 | (AF234984) putative pseudouridine synthase |
| | | | | | | | [Arabidopsis thaliana] |
| 1463 | LIB3602-070-Q1-K1-A4 | 7239379 | BLASTX | 653 | 2.00E−68 | 66 | (AF234984) putative pseudouridine synthase |
| | | | | | | | [Arabidopsis thaliana] |
| 1464 | LIB3602-074-Q1-K1-C7 | 7239379 | BLASTX | 577 | 2.00E−59 | 73 | (AF234984) putative pseudouridine synthase |
| | | | | | | | [Arabidopsis thaliana] |
| 1465 | LIB3602-105-Q1-K1-B9 | 7239379 | BLASTX | 349 | 5.00E−53 | 72 | (AF234984) putative pseudouridine synthase |
| | | | | | | | [Arabidopsis thaliana] |
| 1466 | LIB3602-067-Q1-K1-A3 | 6093871 | BLASTX | 724 | 1.00E−76 | 74 | 60S RIBOSOMAL PROTEIN L15-1 |
| | | | | | | | >gi\|2982249\|gb\|AAC32112.1\| (AF051207) probable |
| | | | | | | | 60S ribosomal protein L15 [Picea mariana] |
| 1467 | LIB3602-028-Q6-K1-B1 | 6093871 | BLASTX | 676 | 4.00E−71 | 72 | 60S RIBOSOMAL PROTEIN L15-1 |
| | | | | | | | >gi\|2982249\|gb\|AAC32112.1\| (AF051207) probable |
| | | | | | | | 60S ribosomal protein L15 [Picea mariana] |
| 1468 | LIB3602-044-Q6-K1-A9 | 6093871 | BLASTX | 621 | 1.00E−64 | 73 | 60S RIBOSOMAL PROTEIN L15-1 |
| | | | | | | | >gi\|2982249\|gb\|AAC32112.1\| (AF051207) probable |
| | | | | | | | 60S ribosomal protein L15 [Picea mariana] |
| 1469 | LIB3602-015-Q6-K1-A9 | 6093871 | BLASTX | 346 | 4.00E−40 | 58 | 60S RIBOSOMAL PROTEIN L15-1 |
| | | | | | | | >gi\|2982249\|gb\|AAC32112.1\| (AF051207) probable |
| | | | | | | | 60S ribosomal protein L15 [Picea mariana] |
| 1470 | LIB3602-030-Q6-K1-G7 | 115828 | BLASTX | 438 | 3.00E−43 | 76 | CHLOROPHYLL A-B BINDING PROTEIN OF |
| | | | | | | | LHCII TYPE I PRECURSOR (CAB) (LHCP) |
| | | | | | | | >gi\|81223\|pir\|\|JS0172 chlorophyll a/b-binding |
| | | | | | | | protein precursor - green alga (Dunaliella salina) |
| | | | | | | | >gi\|167987\|gb\|AAA33278.1\| (M23531) major |
| | | | | | | | chlorophyll binding protein [Dunaliella salina |
| 1471 | LIB3602-037-Q6-K1-E7 | 115828 | BLASTX | 438 | 3.00E−43 | 76 | CHLOROPHYLL A-B BINDING PROTEIN OF |
| | | | | | | | LHCII TYPE I PRECURSOR (CAB) (LHCP) |
| | | | | | | | >gi\|81223\|pir\|\|JS0172 chlorophyll a/b-binding |
| | | | | | | | protein precursor - green alga (Dunaliella salina) |
| | | | | | | | >gi\|167987\|gb\|AAA33278.1\| (M23531) major |
| | | | | | | | chlorophyll binding protein [Dunaliella salina |
| 1472 | LIB3602-020-Q6-K1-G11 | 115828 | BLASTX | 427 | 7.00E−42 | 76 | CHLOROPHYLL A-B BINDING PROTEIN OF |
| | | | | | | | LHCII TYPE I PRECURSOR (CAB) (LHCP) |
| | | | | | | | >gi\|81223\|pir\|\|JS0172 chlorophyll a/b-binding |
| | | | | | | | protein precursor - green alga (Dunaliella salina) |
| | | | | | | | >gi\|167987\|gb\|AAA33278.1\| (M23531) major |
| | | | | | | | chlorophyll binding protein [Dunaliella salina |
| 1473 | LIB3602-101-Q1-K1-D10 | 115828 | BLASTX | 393 | 8.00E−38 | 75 | CHLOROPHYLL A-B BINDING PROTEIN OF |
| | | | | | | | LHCII TYPE I PRECURSOR (CAB) (LHCP) |
| | | | | | | | >gi\|81223\|pir\|\|JS0172 chlorophyll a/b-binding |
| | | | | | | | protein precursor - green alga (Dunaliella salina) |
| | | | | | | | >gi\|167987\|gb\|AAA33278.1\| (M23531) major |
| | | | | | | | chlorophyll binding protein [Dunaliella salina |
| 1474 | LIB3602-012-Q6-K1-D1 | 115770 | BLASTX | 672 | 2.00E−70 | 79 | CHLOROPHYLL A-B BINDING PROTEIN OF |
| | | | | | | | LHCII TYPE I PRECURSOR (CAB) (LHCP) |
| | | | | | | | >gi\|100533\|pir\|\|S07448 chlorophyll a/b-binding |
| | | | | | | | protein - swollen duckweed |
| | | | | | | | >gi\|168290\|gb\|AAA33392.1\| (M12152) chlorophyll |
| | | | | | | | a/b apoprotein [Lemna gibba] |
| 1475 | LIB3602-086-Q6-K1-A11 | 115770 | BLASTX | 544 | 8.00E−70 | 81 | CHLOROPHYLL A-B BINDING PROTEIN OF |
| | | | | | | | LHCII TYPE I PRECURSOR (CAB) (LHCP) |
| | | | | | | | >gi\|100533\|pir\|\|S07448 chlorophyll a/b-binding |
| | | | | | | | protein - swollen duckweed |
| | | | | | | | >gi\|168290\|gb\|AAA33392.1\| (M12152) chlorophyll |
| | | | | | | | a/b apoprotein [Lemna gibba] |
| 1476 | LIB3602-018-Q6-K1-C8 | 115770 | BLASTX | 650 | 5.00E−68 | 78 | CHLOROPHYLL A-B BINDING PROTEIN OF |
| | | | | | | | LHCII TYPE I PRECURSOR (CAB) (LHCP) |
| | | | | | | | >gi\|100533\|pir\|\|S07448 chlorophyll a/b-binding |
| | | | | | | | protein - swollen duckweed |
| | | | | | | | >gi\|168290\|gb\|AAA33392.1\| (M12152) chlorophyll |
| | | | | | | | a/b apoprotein [Lemna gibba] |
| 1477 | LIB3602-048-Q6-K1QA-E1 | 115770 | BLASTX | 606 | 7.00E−63 | 73 | CHLOROPHYLL A-B BINDING PROTEIN OF |
| | | | | | | | LHCII TYPE I PRECURSOR (CAB) (LHCP) |
| | | | | | | | >gi\|100533\|pir\|\|S07448 chlorophyll a/b-binding |
| | | | | | | | protein - swollen duckweed |
| | | | | | | | >gi\|168290\|gb\|AAA33392.1\| (M12152) chlorophyll |
| | | | | | | | a/b apoprotein [Lemna gibba] |
| 1478 | LIB3602-048-Q6-K1QA-C9 | 6682246 | BLASTX | 404 | 3.00E−39 | 93 | (AC016661) putative 40S ribosomal protein S23 |
| | | | | | | | [Arabidopsis thaliana] |
| 1479 | LIB3602-101-Q1-K1-F10 | 6682246 | BLASTX | 347 | 1.00E−32 | 91 | (AC016661) putative 40S ribosomal protein S23 |
| | | | | | | | [Arabidopsis thaliana] |
| 1480 | LIB3602-077-Q6-K6-A4 | 6682246 | BLASTX | 284 | 2.00E−25 | 91 | (AC016661) putative 40S ribosomal protein S23 |
| | | | | | | | [Arabidopsis thaliana] |
| 1481 | LIB3602-074-Q1-K1-C5 | 1350995 | BLASTX | 714 | 2.00E−75 | 68 | 40S RIBOSOMAL PROTEIN S4 |
| | | | | | | | >gi\|1362730\|pir\|\|A56537 ribosomal protein S4, |
| | | | | | | | cytosolic - chicken >gi\|402296\|gb\|AAB59946.1\| |
| | | | | | | | (L24368) ribosomal protein S4 [Gallus gallus] |
| 1482 | LIB3602-023-Q6-K1-G1 | 1350995 | BLASTX | 674 | 8.00E−71 | 70 | 40S RIBOSOMAL PROTEIN S4 |
| | | | | | | | >gi\|1362730\|pir\|\|A56537 ribosomal protein S4, |
| | | | | | | | cytosolic - chicken >gi\|402296\|gb\|AAB59946.1\| |
| | | | | | | | (L24368) ribosomal protein S4 [Gallus gallus] |
| 1483 | LIB3602-061-Q6-K1-D8 | 1350995 | BLASTX | 535 | 1.00E−54 | 62 | 40S RIBOSOMAL PROTEIN S4 |
| | | | | | | | >gi\|1362730\|pir\|\|A56537 ribosomal protein S4, |
| | | | | | | | cytosolic - chicken >gi\|402296\|gb\|AAB59946.1\| |
| | | | | | | | (L24368) ribosomal protein S4 [Gallus gallus] |
| 1484 | LIB3602-046-Q6-K1-H5 | 7485005 | BLASTX | 382 | 1.00E−36 | 59 | helicase homolog T6H20.10 - Arabidopsis thaliana |
| | | | | | | | (fragment) >gi\|5541663\|emb\|CAB51169.1\| |
| | | | | | | | (AL096859) putative helicase, fragment |
| | | | | | | | [Arabidopsis thaliana] |
| 1485 | LIB3602-112-Q1-K1-B9 | 7485005 | BLASTX | 319 | 3.00E−29 | 66 | helicase homolog T6H20.10 - Arabidopsis thaliana |
| | | | | | | | (fragment) >gi\|5541663\|emb\|CAB51169.1\| |
| | | | | | | | (AL096859) putative helicase, fragment |
| | | | | | | | [Arabidopsis thaliana] |
| 1486 | LIB3602-094-Q6-K6-B9 | 7485005 | BLASTX | 310 | 4.00E−28 | 64 | helicase homolog T6H20.10 - Arabidopsis thaliana |
| | | | | | | | (fragment) >gi\|5541663\|emb\|CAB51169.1\| |
| | | | | | | | (AL096859) putative helicase, fragment |
| | | | | | | | [Arabidopsis thaliana] |
| 1487 | LIB3602-076-Q6-K6-D10 | 6587821 | BLASTX | 230 | 6.00E−19 | 57 | (AC010924) Contains similarity to gb\|AF181686 |
| | | | | | | | membrane protein TMS1d from Drosophila |
| | | | | | | | melanogaster. ESTs gb\|R64994, gb\|AI994832, |
| | | | | | | | gb\|Z47674 come from this gene. [Arabidopsis |
| | | | | | | | thaliana] |
| 1488 | LIB3602-070-Q1-K1-A9 | 6587821 | BLASTX | 203 | 1.00E−15 | 56 | (AC010924) Contains similarity to gb\|AF181686 |
| | | | | | | | membrane protein TMS1d from Drosophila |
| | | | | | | | melanogaster. ESTs gb\|R64994, gb\|AI994832, |
| | | | | | | | gb\|Z47674 come from this gene. [Arabidopsis |
| | | | | | | | thaliana] |
| 1489 | LIB3602-010-Q6-K1-E4 | 6587821 | BLASTX | 190 | 3.00E−14 | 36 | (AC010924) Contains similarity to gb\|AF181686 |
| | | | | | | | membrane protein TMS1d from Drosophila |
| | | | | | | | melanogaster. ESTs gb\|R64994, gb\|AI994832, |
| | | | | | | | gb\|Z47674 come from this gene. [Arabidopsis |
| | | | | | | | thaliana] |
| 1490 | LIB3602-116-Q1-K1-A5 | 4139217 | BLASTN | 61 | 9.00E−26 | 92 | Chlamydomonas reinhardtii light harvesting |
| | | | | | | | complex II protein precursor (Lhcb3) mRNA, |
| | | | | | | | complete cds |
| 1491 | LIB3602-004-Q1-K1-B3 | 4139217 | BLASTN | 51 | 2.00E−19 | 92 | Chlamydomonas reinhardtii light harvesting |
| | | | | | | | complex II protein precursor (Lhcb3) mRNA, |
| | | | | | | | complete cds |
| 1492 | LIB3602-063-Q1-K6-B8 | 4139217 | BLASTN | 42 | 5.00E−14 | 96 | Chlamydomonas reinhardtii light harvesting |
| | | | | | | | complex II protein precursor (Lhcb3) mRNA, |
| | | | | | | | complete cds |
| 1493 | LIB3602-009-Q6-K1-D7 | 4262250 | BLASTX | 691 | 8.00E−73 | 78 | (AC006200) putative fructose-bisphosphate |
| | | | | | | | aldolase, plastidic form [Arabidopsis thaliana] |
| 1494 | LIB3602-004-Q1-K1-C1 | 4262250 | BLASTX | 563 | 5.00E−63 | 73 | (AC006200) putative fructose-bisphosphate |
| | | | | | | | aldolase, plastidic form [Arabidopsis thaliana] |
| 1495 | LIB3602-043-Q6-K1-A8 | 4262250 | BLASTX | 473 | 3.00E−47 | 72 | (AC006200) putative fructose-bisphosphate |
| | | | | | | | aldolase, plastidic form [Arabidopsis thaliana] |
| 1496 | LIB3602-009-Q6-K1-B1 | 6996529 | BLASTX | 556 | 5.00E−57 | 71 | (AJ271785) enolase [Lupinus luteus] |
| 1497 | LIB3602-037-Q6-K1-G2 | 6996529 | BLASTX | 249 | 4.00E−21 | 85 | (AJ271785) enolase [Lupinus luteus] |
| 1498 | LIB3602-015-Q6-K1-H10 | 6996529 | BLASTX | 211 | 2.00E−16 | 89 | (AJ271785) enolase [Lupinus luteus] |
| 1499 | LIB3602-023-Q6-K1-A3 | 2708624 | BLASTX | 615 | 6.00E−64 | 66 | (AF036618) acetyl-CoA synthetase [Arabidopsis |
| | | | | | | | thaliana] |
| 1500 | LIB3602-108-Q1-K1-G5 | 2708624 | BLASTX | 595 | 1.00E−61 | 69 | (AF036618) acetyl-CoA synthetase [Arabidopsis |
| | | | | | | | thaliana] |
| 1501 | LIB3602-119-Q1-K1-F11 | 2708624 | BLASTX | 166 | 5.00E−12 | 71 | (AF036618) acetyl-CoA synthetase [Arabidopsis |
| | | | | | | | thaliana] |
| 1502 | LIB3602-063-Q1-K6-F4 | 131174 | BLASTX | 272 | 4.00E−24 | 77 | PHOTOSYSTEM I REACTION CENTRE |
| | | | | | | | SUBUNIT IV PRECURSOR (PHOTOSYSTEM I |
| | | | | | | | 8.1 KD PROTEIN) (P30 PROTEIN) (PSI-E) |
| | | | | | | | >gi\|81245\|pir\|\|S04134 photosystem I chain IV |
| | | | | | | | precursor - Chlamydomonas reinhardtii |
| | | | | | | | >gi\|18180\|emb\|CAA31850.1\| (X13496) P30 |
| | | | | | | | precursor protein [Chlamydomo |
| 1503 | LIB3602-070-Q1-K1-E6 | 131174 | BLASTX | 268 | 2.00E−23 | 78 | PHOTOSYSTEM I REACTION CENTRE |
| | | | | | | | SUBUNIT IV PRECURSOR (PHOTOSYSTEM I |
| | | | | | | | 8.1 KD PROTEIN) (P30 PROTEIN) (PSI-E) |
| | | | | | | | >gi\|81245\|pir\|\|S04134 photosystem I chain IV |
| | | | | | | | precursor - Chlamydomonas reinhardtii |
| | | | | | | | >gi\|18180\|emb\|CAA31850.1\| (X13496) P30 |
| | | | | | | | precursor protein [Chlamydomo |
| 1504 | LIB3602-021-Q6-K1-A5 | 131174 | BLASTX | 219 | 6.00E−18 | 78 | PHOTOSYSTEM I REACTION CENTRE |
| | | | | | | | SUBUNIT IV PRECURSOR (PHOTOSYSTEM I |
| | | | | | | | 8.1 KD PROTEIN) (P30 PROTEIN) (PSI-E) |
| | | | | | | | >gi\|81245\|pir\|\|S04134 photosystem I chain IV |
| | | | | | | | precursor - Chlamydomonas reinhardtii |
| | | | | | | | >gi\|18180\|emb\|CAA31850.1\| (X13496) P30 |
| | | | | | | | precursor protein [Chlamydomo |
| 1505 | LIB3602-066-Q1-K6-D9 | 1345698 | BLASTX | 528 | 9.00E−54 | 68 | CHLOROPHYLL A-B BINDING PROTEIN 151 |
| | | | | | | | PRECURSOR (LHCII TYPE II CAB-151) (LHCP) |
| | | | | | | | >gi\|99601\|pir\|\|S20917 chlorophyll a/b-binding |
| | | | | | | | protein - upland cotton |
| | | | | | | | >gi\|452314\|emb\|CAA38025.1\| (X54090) |
| | | | | | | | chlorophyll ab binding protein [Gossypium |
| | | | | | | | hirsutum] |
| 1506 | LIB3602-065-Q1-K6-B9 | 1345698 | BLASTX | 418 | 2.00E−48 | 71 | CHLOROPHYLL A-B BINDING PROTEIN 151 |
| | | | | | | | PRECURSOR (LHCII TYPE II CAB-151) (LHCP) |
| | | | | | | | >gi\|99601\|pir\|\|S20917 chlorophyll a/b-binding |
| | | | | | | | protein - upland cotton |
| | | | | | | | >gi\|452314\|emb\|CAA38025.1\| (X54090) |
| | | | | | | | chlorophyll ab binding protein [Gossypium |
| | | | | | | | hirsutum] |
| 1507 | LIB3602-053-Q6-K1-C1 | 1345698 | BLASTX | 301 | 4.00E−27 | 78 | CHLOROPHYLL A-B BINDING PROTEIN 151 |
| | | | | | | | PRECURSOR (LHCII TYPE II CAB-151) (LHCP) |
| | | | | | | | >gi\|99601\|pir\|\|S20917 chlorophyll a/b-binding |
| | | | | | | | protein - upland cotton |
| | | | | | | | >gi\|452314\|emb\|CAA38025.1\| (X54090) |
| | | | | | | | chlorophyll ab binding protein [Gossypium |
| | | | | | | | hirsutum] |
| 1508 | LIB3602-069-Q1-K1-D2 | 7329685 | BLASTX | 306 | 1.00E−27 | 69 | (AL162295) transketolase-like protein [Arabidopsis |
| | | | | | | | thaliana] |
| 1509 | LIB3602-086-Q6-K1-G1 | 7329685 | BLASTX | 306 | 1.00E−27 | 69 | (AL162295) transketolase-like protein [Arabidopsis |
| | | | | | | | thaliana] |
| 1510 | LIB3602-026-Q6-K1-E6 | 7329685 | BLASTX | 231 | 6.00E−19 | 68 | (AL162295) transketolase-like protein [Arabidopsis |
| | | | | | | | thaliana] |
| 1511 | LIB3602-010-Q6-K1-D3 | 115519 | BLASTX | 667 | 5.00E−70 | 85 | CALMODULIN>gi\|169306\|gb\|AAA21424.1\| |
| | | | | | | | (M83535) calmodulin [Phytophthora infestans] |
| 1512 | LIB3602-039-Q6-K1-E10 | 115519 | BLASTX | 634 | 4.00E−66 | 87 | CALMODULIN >gi\|169306\|gb\|AAA21424.1\| |
| | | | | | | | (M83535) calmodulin [Phytophthora infestans] |
| 1513 | LIB3602-019-Q6-K1-C5 | 115519 | BLASTX | 349 | 5.00E−33 | 84 | CALMODULIN >gi\|169306\|gb\|AAA21424.1\| |
| | | | | | | | (M83535) calmodulin [Phytophthora infestans] |
| 1514 | LIB3602-004-Q1-K1-C4 | 100573 | BLASTX | 363 | 2.00E−34 | 48 | chlorophyll a/b-binding protein CP29 precursor - |
| | | | | | | | barley >gi\|18958\|emb\|CAA44777.1\| (X63052) |
| | | | | | | | Precursor of CP29, core chlorophyll a/b binding |
| | | | | | | | (CAB) protein of photosystem II (PSII) [Hordeum |
| | | | | | | | vulgare] >gi\|445122\|prf\|\|1908428A chlorophyll a/b- |
| | | | | | | | binding protein [ |
| 1515 | LIB3602-021-Q6-K1-B4 | 100573 | BLASTX | 293 | 1.00E−26 | 64 | chlorophyll a/b-binding protein CP29 precursor - |
| | | | | | | | barley >gi\|18958\|emb\|CAA44777.1\| (X63052) |
| | | | | | | | Precursor of CP29, core chlorophyll a/b binding |
| | | | | | | | (CAB) protein of photosystem II (PSII) [Hordeum |
| | | | | | | | vulgare] >gi\|445122\|prf\|\|1908428A chlorophyll a/b- |
| | | | | | | | binding protein [ |
| 1516 | LIB3602-052-Q6-K1-B10 | 100573 | BLASTX | 185 | 6.00E−14 | 60 | chlorophyll a/b-binding protein CP29 precursor - |
| | | | | | | | barley >gi\|18958\|emb\|CAA44777.1\| (X63052) |
| | | | | | | | Precursor of CP29, core chlorophyll a/b binding |
| | | | | | | | (CAB) protein of photosystem II (PSII) [Hordeum |
| | | | | | | | vulgare] >gi\|445122\|prf\|\|1908428A chlorophyll a/b- |
| | | | | | | | binding protein [ |
| 1517 | LIB3602-085-Q6-K1-D12 | 548770 | BLASTX | 629 | 1.00E−65 | 71 | 60S RIBOSOMAL PROTEIN L3 |
| | | | | | | | >gi\|481228\|pir\|\|S38359 ribosomal protein L3.e, |
| | | | | | | | cytosolic - rice >gi\|303853\|dbj\|BAA02155.1\| |
| | | | | | | | (D12630) ribosomal protein L3 [Oryza sativa] |
| 1518 | LIB3602-013-Q6-K1-B12 | 548770 | BLASTX | 607 | 4.00E−63 | 75 | 60S RIBOSOMAL PROTEIN L3 |
| | | | | | | | >gi\|481228\|pir\|\|S38359 ribosomal protein L3.e, |
| | | | | | | | cytosolic - rice >gi\|303853\|dbj\|BAA02155.1\| |
| | | | | | | | (D12630) ribosomal protein L3 [Oryza sativa] |
| 1519 | LIB3602-112-Q1-K1-H12 | 548770 | BLASTX | 259 | 3.00E−45 | 59 | 60S RIBOSOMAL PROTEIN L3 |
| | | | | | | | >gi\|481228\|pir\|\|S38359 ribosomal protein L3.e, |
| | | | | | | | cytosolic - rice >gi\|303853\|dbj\|BAA02155.1\| |
| | | | | | | | (D12630) ribosomal protein L3 [Oryza sativa] |
| 1520 | LIB3602-069-Q1-K1-A12 | 1663720 | BLASTX | 528 | 1.00E−53 | 57 | (U49976) beta-type carbonic anhydrase beta-CA1 |
| | | | | | | | [Coccomyxa sp. PA] |
| 1521 | LIB3602-068-Q1-K1-A12 | 1663720 | BLASTX | 519 | 1.00E−52 | 56 | (U49976) beta-type carbonic anhydrase beta-CA1 |
| | | | | | | | [Coccomyxa sp. PA] |
| 1522 | LIB3602-062-Q6-K1-G1 | 1663720 | BLASTX | 216 | 4.00E−17 | 53 | (U49976) beta-type carbonic anhydrase beta-CA1 |
| | | | | | | | [Coccomyxa sp. PA] |
| 1523 | LIB3602-064-Q1-K6-G5 | 6440928 | BLASTX | 790 | 2.00E−84 | 71 | (AU066544) 60S ribosomal protein L2 |
| | | | | | | | [Chlamydomonas sp.] |
| 1524 | LIB3602-055-Q6-K1-E11 | 6440928 | BLASTX | 636 | 2.00E−66 | 80 | (AU066544) 60S ribosomal protein L2 |
| | | | | | | | [Chlamydomonas sp.] |
| 1525 | LIB3602-116-Q1-K1-C10 | 6440928 | BLASTX | 487 | 1.00E−59 | 67 | (AU066544) 60S ribosomal protein L2 |
| | | | | | | | [Chlamydomonas sp.] |
| 1526 | LIB3602-074-Q1-K1-B2 | 5734785 | BLASTX | 879 | 8.00E−95 | 81 | (AC007980) phosphoprotein phosphatase |
| | | | | | | | [Arabidopsis thaliana] |
| 1527 | LIB3602-107-Q1-K1-B5 | 5734785 | BLASTX | 852 | 1.00E−91 | 84 | (AC007980) phosphoprotein phosphatase |
| | | | | | | | [Arabidopsis thaliana] |
| 1528 | LIB3602-062-Q6-K1-H9 | 5734785 | BLASTX | 409 | 2.00E−75 | 73 | (AC007980) phosphoprotein phosphatase |
| | | | | | | | [Arabidopsis thaliana] |
| 1529 | LIB3602-028-Q6-K1-C4 | 131199 | BLASTX | 201 | 2.00E−15 | 42 | PHOTOSYSTEM I REACTION CENTRE |
| | | | | | | | SUBUNIT VI PRECURSOR (LIGHT- |
| | | | | | | | HARVESTING COMPLEX I 11 KD PROTEIN) |
| | | | | | | | (PSI-H) >gi\|81470\|pir\|\|S00453 photosystem I |
| | | | | | | | protein psaH precursor - spinach |
| | | | | | | | >gi\|21287\|emb\|CAA34749.1\| (X16858) psaH |
| | | | | | | | [Spinacia oleracea] |
| 1530 | LIB3602-014-Q6-K1-F2 | 131199 | BLASTX | 179 | 8.00E−13 | 40 | PHOTOSYSTEM I REACTION CENTRE |
| | | | | | | | SUBUNIT VI PRECURSOR (LIGHT- |
| | | | | | | | HARVESTING COMPLEX I 11 KD PROTEIN) |
| | | | | | | | (PSI-H) >gi\|81470\|pir\|\|S00453 photosystem I |
| | | | | | | | protein psaH precursor - spinach |
| | | | | | | | >gi\|21287\|emb\|CAA34749.1\| (X16858) psaH |
| | | | | | | | [Spinacia oleracea] |
| 1531 | LIB3602-108-Q1-K1-G10 | 131199 | BLASTX | 149 | 1.00E−11 | 37 | PHOTOSYSTEM I REACTION CENTRE |
| | | | | | | | SUBUNIT VI PRECURSOR (LIGHT- |
| | | | | | | | HARVESTING COMPLEX I 11 KD PROTEIN) |
| | | | | | | | (PSI-H) >gi\|81470\|pir\|\|S00453 photosystem I |
| | | | | | | | protein psaH precursor - spinach |
| | | | | | | | >gi\|21287\|emb\|CAA34749.1\| (X16858) psaH |
| | | | | | | | [Spinacia oleracea] |
| 1532 | LIB3602-039-Q6-K1-G3 | 7630009 | BLASTX | 172 | 1.00E−12 | 63 | (AL132960) 40S ribosomal protein S21 homolog |
| | | | | | | | [Arabidopsis thaliana] |
| 1533 | LIB3602-026-Q6-K1-C6 | 7630009 | BLASTX | 165 | 2.00E−11 | 60 | (AL132960) 40S ribosomal protein S21 homolog |
| | | | | | | | [Arabidopsis thaliana] |
| 1534 | LIB3602-012-Q6-K1-A9 | 7630009 | BLASTX | 153 | 4.00E−10 | 60 | (AL132960) 40S ribosomal protein S21 homolog |
| | | | | | | | [Arabidopsis thaliana] |
| 1535 | LIB3602-077-Q6-K6-D7 | 6647427 | BLASTX | 547 | 7.00E−56 | 73 | ACETYL-COENZYME A SYNTHETASE |
| | | | | | | | (ACETATE-COA LIGASE) (ACYL- |
| | | | | | | | ACTIVATING ENZYME) >gi\|7437830\|pir\|\|T03473 |
| | | | | | | | acetate - CoA ligase (EC 6.2.1.1) - Rhodobacter |
| | | | | | | | capsulatus >gi\|3128274\|gb\|AAC16126.1\| |
| | | | | | | | (AF010496) acetyl-coenzyme a synthetase |
| | | | | | | | [Rhodobacter capsulatus] |
| 1536 | LIB3602-090-Q6-K6-E5 | 6647427 | BLASTX | 280 | 1.00E−24 | 69 | ACETYL-COENZYME A SYNTHETASE |
| | | | | | | | (ACETATE--COA LIGASE) (ACYL- |
| | | | | | | | ACTIVATING ENZYME) >gi\|7437830\|pir\|\|T03473 |
| | | | | | | | acetate - CoA ligase (EC 6.2.1.1) - Rhodobacter |
| | | | | | | | capsulatus >gi\|3128274\|gb\|AAC16126.1\| |
| | | | | | | | (AF010496) acetyl-coenzyme a synthetase |
| | | | | | | | [Rhodobacter capsulatus] |
| 1537 | LIB3602-023-Q6-K1-E3 | 6647427 | BLASTX | 256 | 8.00E−22 | 69 | ACETYL-COENZYME A SYNTHETASE |
| | | | | | | | (ACETATE-COA LIGASE) (ACYL- |
| | | | | | | | ACTIVATING ENZYME) >gi\|7437830\|pir\|\|T03473 |
| | | | | | | | acetate - CoA ligase (EC 6.2.1.1) - Rhodobacter |
| | | | | | | | capsulatus >gi\|3128274\|gb\|AAC16126.1\| |
| | | | | | | | (AF010496) acetyl-coenzyme a synthetase |
| | | | | | | | [Rhodobacter capsulatus] |
| 1538 | LIB3602-010-Q6-K1-G9 | 6325315 | BLASTX | 174 | 3.00E−12 | 36 | putative mitochondrial carrier protein; Ymc1p |
| | | | | | | | >gi\|1084942\|pir\|\|S54080 carrier protein YMC1, |
| | | | | | | | mitochondrial - yeast (Saccharomyces cerevisiae) |
| | | | | | | | >gi\|805039\|emb\|CAA89176.1\| (Z49219) Ymc1p |
| | | | | | | | [Saccharomyces cerevisiae] |
| | | | | | | | >gi\|1314127\|emb\|CAA95003.1\| (Z71255) Ymc1p |
| | | | | | | | [S |
| 1539 | LIB3602-040-Q6-K1-F3 | 6325315 | BLASTX | 173 | 5.00E−12 | 38 | putative mitochondrial carrier protein; Ymc1p |
| | | | | | | | >gi\|1084942\|pir\|\|S54080 carrier protein YMC1, |
| | | | | | | | mitochondrial - yeast (Saccharomyces cerevisiae) |
| | | | | | | | >gi\|805039\|emb\|CAA89176.1\| (Z49219) Ymc1p |
| | | | | | | | [Saccharomyces cerevisiae] |
| | | | | | | | >gi\|1314127\|emb\|CAA95003.1\| (Z71255) Ymc1p |
| | | | | | | | [S |
| 1540 | LIB3602-070-Q1-K1-A6 | 6325315 | BLASTX | 170 | 1.00E−11 | 37 | putative mitochondrial carrier protein; Ymc1p |
| | | | | | | | >gi\|1084942\|pir\|\|S54080 carrier protein YMC1, |
| | | | | | | | mitochondrial - yeast (Saccharomyces cerevisiae) |
| | | | | | | | >gi\|805039\|emb\|CAA89176.1\| (Z49219) Ymc1p |
| | | | | | | | [Saccharomyces cerevisiae] |
| | | | | | | | >gi\|1314127\|emb\|CAA95003.1\| (Z71255) Ymc1p |
| | | | | | | | [S |
| 1541 | LIB3602-094-Q6-K6-D7 | 6093778 | BLASTX | 733 | 1.00E−77 | 76 | PROTEASOME COMPONENT C3 (MACROPAIN |
| | | | | | | | SUBUNIT C3) (MULTICATALYTIC |
| | | | | | | | ENDOPEPTIDASE COMPLEX SUBUNIT C3) |
| | | | | | | | >gi\|2511574\|emb\|CAA73619.1\| (Y13176) |
| | | | | | | | multicatalytic endopeptidase [Arabidopsis thaliana] |
| | | | | | | | >gi\|3421075\|gb\|AAC32056.1\| (AF043520) 20S |
| | | | | | | | proteasome subunit PAB1 [Ar |
| 1542 | LIB3602-088-Q6-K6-G6 | 6093778 | BLASTX | 709 | 7.00E−75 | 76 | PROTEASOME COMPONENT C3 (MACROPAIN |
| | | | | | | | SUBUNIT C3) (MULTICATALYTIC |
| | | | | | | | ENDOPEPTIDASE COMPLEX SUBUNIT C3) |
| | | | | | | | >gi\|2511574\|emb\|CAA73619.1\| (Y13176) |
| | | | | | | | multicatalytic endopeptidase [Arabidopsis thaliana] |
| | | | | | | | >gi\|3421075\|gb\|AAC32056.1\| (AF043520) 20S |
| | | | | | | | proteasome subunit PAB1 [Ar |
| 1543 | LIB3602-118-Q1-K1-G6 | 6093778 | BLASTX | 545 | 2.00E−68 | 76 | PROTEASOME COMPONENT C3 (MACROPAIN |
| | | | | | | | SUBUNIT C3) (MULTICATALYTIC |
| | | | | | | | ENDOPEPTIDASE COMPLEX SUBUNIT C3) |
| | | | | | | | >gi\|2511574\|emb\|CAA73619.1\| (Y13176) |
| | | | | | | | multicatalytic endopeptidase [Arabidopsis thaliana] |
| | | | | | | | >gi\|3421075\|gb\|AAC32056.1\| (AF043520) 20S |
| | | | | | | | proteasome subunit PAB1 [Ar |
| 1544 | LIB3602-005-Q1-K1-A9 | 4557867 | BLASTX | 607 | 7.00E−63 | 59 | sulfite oxidase |
| | | | | | | | >gi\|1711606\|sp\|P51687\|SUOX_HUMAN SULFITE |
| | | | | | | | OXIDASE PRECURSOR >gi\|1362942\|pir\|\|S55874 |
| | | | | | | | sulfite oxidase (EC 1.8.3.1) precursor, |
| | | | | | | | mitochondrial - human >gi\|508502\|gb\|AAA74886.1\| |
| | | | | | | | (L31573) sulfite oxidase [Homo sapiens] |
| | | | | | | | >gi\|1098062\|prf\|\|2115221A su |
| 1545 | LIB3602-053-Q6-K1-A10 | 4557867 | BLASTX | 594 | 2.00E−61 | 60 | sulfite oxidase |
| | | | | | | | >gi\|1711606\|sp\|P51687\|SUOX_HUMAN SULFITE |
| | | | | | | | OXIDASE PRECURSOR >gi\|1362942\|pir\|\|S55874 |
| | | | | | | | sulfite oxidase (EC 1.8.3.1) precursor, |
| | | | | | | | mitochondrial - human >gi\|508502\|gb\|AAA74886.1\| |
| | | | | | | | (L31573) sulfite oxidase [Homo sapiens] |
| | | | | | | | >gi\|1098062\|prf\|\|2115221A su |
| 1546 | LIB3602-105-Q1-K1-C8 | 4557867 | BLASTX | 172 | 6.00E−12 | 45 | sulfite oxidase |
| | | | | | | | >gi\|1711606\|sp\|P51687\|SUOX_HUMAN SULFITE |
| | | | | | | | OXIDASE PRECURSOR >gi\|1362942\|pir\|\|S55874 |
| | | | | | | | sulfite oxidase (EC 1.8.3.1) precursor, |
| | | | | | | | mitochondrial - human >gi\|508502\|gb\|AAA74886.1\| |
| | | | | | | | (L31573) sulfite oxidase [Homo sapiens] |
| | | | | | | | >gi\|1098062\|prf\|\|2115221A su |
| 1547 | LIB3602-118-Q1-K1-A3 | 2688826 | BLASTX | 185 | 1.00E−13 | 52 | (U95179) putative NifU protein [Prunus armeniaca] |
| 1548 | LIB3602-095-Q6-K6-F3 | 2688826 | BLASTX | 185 | 2.00E−13 | 52 | (U95179) putative NifU protein [Prunus armeniaca] |
| 1549 | LIB3602-120-Q1-K1-D7 | 2688826 | BLASTX | 179 | 9.00E−13 | 50 | (U95179) putative NifU protein [Prunus armeniaca] |
| 1550 | LIB3602-046-Q6-K1-A2 | 2583111 | BLASTX | 456 | 2.00E−45 | 58 | (AC002387) putative dihydrodipicolinate synthase |
| | | | | | | | [Arabidopsis thaliana] |
| 1551 | LIB3602-012-Q6-K1-A8 | 2583111 | BLASTX | 430 | 4.00E−42 | 53 | (AC002387) putative dihydrodipicolinate synthase |
| | | | | | | | [Arabidopsis thaliana] |
| 1552 | LIB3602-028-Q6-K1-C2 | 2583111 | BLASTX | 425 | 1.00E−41 | 70 | (AC002387) putative dihydrodipicolinate synthase |
| | | | | | | | [Arabidopsis thaliana] |
| 1553 | LIB3602-056-Q6-K1-H9 | 1350996 | BLASTX | 515 | 3.00E−52 | 66 | 40S RIBOSOMAL PROTEIN S4 |
| | | | | | | | >gi\|453574\|dbj\|BAA05485.1\| (D26473) Ribosomal |
| | | | | | | | Protein [Cricetulus griseus] |
| 1554 | LIB3602-116-Q1-K1-B12 | 1350996 | BLASTX | 338 | 5.00E−47 | 66 | 40S RIBOSOMAL PROTEIN S4 |
| | | | | | | | >gi\|453574\|dbj\|BAA05485.1\| (D26473) Ribosomal |
| | | | | | | | Protein [Cricetulus griseus] |
| 1555 | LIB3602-056-Q6-K1-H10 | 1350996 | BLASTX | 236 | 2.00E−24 | 58 | 40S RIBOSOMAL PROTEIN S4 |
| | | | | | | | >gi\|453574\|dbj\|BAA05485.1\| (D26473) Ribosomal |
| | | | | | | | Protein [Cricetulus griseus] |
| 1556 | LIB3602-032-Q6-K1-C6 | 7443230 | BLASTX | 245 | 9.00E−21 | 52 | probable chlorophyll a/b-binding protein - rice |
| | | | | | | | >gi\|3075488\|gb\|AAC14566.1\| (AF058796) |
| | | | | | | | chlorophyll a/b-binding protein [Oryza sativa] |
| 1557 | LIB3602-019-Q6-K1-F11 | 7443230 | BLASTX | 239 | 7.00E−20 | 56 | probable chlorophyll a/b-binding protein - rice |
| | | | | | | | >gi\|3075488\|gb\|AAC14566.1\| (AF058796) |
| | | | | | | | chlorophyll a/b-binding protein [Oryza sativa] |
| 1558 | LIB3602-059-Q6-K1-H2 | 7443230 | BLASTX | 227 | 1.00E−18 | 56 | probable chlorophyll a/b-binding protein - rice |
| | | | | | | | >gi\|3075488\|gb\|AAC14566.1\| (AF058796) |
| | | | | | | | chlorophyll a/b-binding protein [Oryza sativa] |
| 1559 | LIB3602-111-Q1-K1-G6 | 2500380 | BLASTX | 260 | 6.00E−35 | 84 | 60S RIBOSOMAL PROTEIN L44 |
| | | | | | | | >gi\|2119128\|pir\|\|JC4923 ribosomal protein L36a.e, |
| | | | | | | | cytosolic - upland cotton |
| | | | | | | | >gi\|1553129\|gb\|AAB08726.1\| (U64677) ribosomal |
| | | | | | | | protein L44 isoform a [Gossypium hirsutum] |
| | | | | | | | >gi\|1553131\|gb\|AAB08727.1\| (U64678) ribosomal |
| | | | | | | | protein L44 isofor |
| 1560 | LIB3602-119-Q1-K1-D5 | 2500380 | BLASTX | 218 | 8.00E−31 | 76 | 60S RIBOSOMAL PROTEIN L44 |
| | | | | | | | >gi\|2119128\|pir\|\|JC4923 ribosomal protein L36a.e, |
| | | | | | | | cytosolic - upland cotton |
| | | | | | | | >gi\|1553129\|gb\|AAB08726.1\| (U64677) ribosomal |
| | | | | | | | protein L44 isoform a [Gossypium hirsutum] |
| | | | | | | | >gi\|1553131\|gb\|AAB08727.1\| (U64678) ribosomal |
| | | | | | | | protein L44 isofor |
| 1561 | LIB3602-065-Q1-K6-A12 | 2500380 | BLASTX | 176 | 1.00E−22 | 63 | 60S RIBOSOMAL PROTEIN L44 |
| | | | | | | | >gi\|2119128\|pir\|\|JC4923 ribosomal protein L36a.e, |
| | | | | | | | cytosolic - upland cotton |
| | | | | | | | >gi\|1553129\|gb\|AAB08726.1\| (U64677) ribosomal |
| | | | | | | | protein L44 isoform a [Gossypium hirsutum] |
| | | | | | | | >gi\|1553131\|gb\|AAB08727.1\| (U64678) ribosomal |
| | | | | | | | protein L44 isofor |
| 1562 | LIB3602-089-Q6-K6-G11 | 7436330 | BLASTX | 444 | 8.00E−44 | 57 | probable H+-transporting ATPase (EC 3.6.1.35) |
| | | | | | | | 18K chain - Arabidopsis thaliana |
| | | | | | | | >gi\|4914450\|emb\|CAB43690.1\| (AL050398) H+- |
| | | | | | | | transporting ATPase-like protein [Arabidopsis |
| | | | | | | | thaliana] >gi\|7270157\|emb\|CAB79970.1\| |
| | | | | | | | (AL161581) H+-transporting ATPase-like protein |
| | | | | | | | [Ar |
| 1563 | LIB3602-095-Q6-K6-G11 | 7436330 | BLASTX | 443 | 1.00E−43 | 56 | probable H+-transporting ATPase (EC 3.6.1.35) |
| | | | | | | | 18K chain - Arabidopsis thaliana |
| | | | | | | | >gi\|4914450\|emb\|CAB43690.1\| (AL050398) H+- |
| | | | | | | | transporting ATPase-like protein [Arabidopsis |
| | | | | | | | thaliana] >gi\|7270157\|emb\|CAB79970.1\| |
| | | | | | | | (AL161581) H+-transporting ATPase-like protein |
| | | | | | | | [Ar |
| 1564 | LIB3602-112-Q1-K1-G11 | 7436330 | BLASTX | 443 | 1.00E−43 | 56 | probable H+-transporting ATPase (EC 3.6.1.35) |
| | | | | | | | 18K chain - Arabidopsis thaliana |
| | | | | | | | >gi\|4914450\|emb\|CAB43690.1\| (AL050398) H+- |
| | | | | | | | transporting ATPase-like protein [Arabidopsis |
| | | | | | | | thaliana] >gi\|7270157\|emb\|CAB79970.1\| |
| | | | | | | | (AL161581) H+-transporting ATPase-like protein |
| | | | | | | | [Ar |
| 1565 | LIB3602-089-Q6-K6-B9 | 6522577 | BLASTX | 310 | 3.00E−28 | 64 | (AL133292) putative helicase [Arabidopsis |
| | | | | | | | thaliana] |
| 1566 | LIB3602-095-Q6-K6-B9 | 6522577 | BLASTX | 310 | 3.00E−28 | 64 | (AL133292) putative helicase [Arabidopsis |
| | | | | | | | thaliana] |
| 1567 | LIB3602-116-Q1-K1-B9 | 6522577 | BLASTX | 195 | 7.00E−26 | 69 | (AL133292) putative helicase [Arabidopsis |
| | | | | | | | thaliana] |
| 1568 | LIB3602-085-Q6-K1-C3 | 4775288 | BLASTX | 285 | 6.00E−36 | 61 | (AJ238628) putative ferritin [Chlorella |
| | | | | | | | protothecoides] |
| 1569 | LIB3602-008-Q6-K1-B6 | 4775288 | BLASTX | 297 | 1.00E−26 | 61 | (AJ238628) putative ferritin [Chlorella |
| | | | | | | | protothecoides] |
| 1570 | LIB3602-112-Q1-K1-C5 | 4775288 | BLASTX | 192 | 3.00E−14 | 55 | (AJ238628) putative ferritin [Chlorella |
| | | | | | | | protothecoides] |
| 1571 | LIB3602-022-Q6-K1-G8 | 421766 | BLASTX | 213 | 7.00E−17 | 63 | chlorophyll a/b-binding protein I-20 precursor - |
| | | | | | | | Chlamydomonas reinhardtii |
| | | | | | | | >gi\|18125\|emb\|CAA46235.1\| (X65119) light |
| | | | | | | | harvesting complex protein I-20 [Chlamydomonas |
| | | | | | | | reinhardtii] |
| 1572 | LIB3602-094-Q6-K6-F1 | 421766 | BLASTX | 208 | 3.00E−16 | 72 | chlorophyll a/b-binding protein I-20 precursor - |
| | | | | | | | Chlamydomonas reinhardtii |
| | | | | | | | >gi\|18125\|emb\|CAA46235.1\| (X65119) light |
| | | | | | | | harvesting complex protein I-20 [Chlamydomonas |
| | | | | | | | reinhardtii] |
| 1573 | LIB3602-104-Q1-K1-E4 | 421766 | BLASTX | 182 | 3.00E−13 | 68 | chlorophyll a/b-binding protein I-20 precursor - |
| | | | | | | | Chlamydomonas reinhardtii |
| | | | | | | | >gi\|18125\|emb\|CAA46235.1\| (X65119) light |
| | | | | | | | harvesting complex protein I-20 [Chlamydomonas |
| | | | | | | | reinhardtii] |
| 1574 | LIB3602-120-Q1-K1-D12 | 1346750 | BLASTX | 665 | 6.00E−92 | 92 | SERINE/THREONINE PROTEIN |
| | | | | | | | PHOSPHATASE PP1 ISOZYME 1 |
| | | | | | | | >gi\|2654372\|emb\|CAA82263.1\| (Z28627) protein |
| | | | | | | | phosphatase 1 [Acetabularia cliftonii] |
| 1575 | LIB3602-107-Q1-K1-A2 | 1346750 | BLASTX | 720 | 3.00E−76 | 86 | SERINE/THREONINE PROTEIN |
| | | | | | | | PHOSPHATASE PP1 ISOZYME 1 |
| | | | | | | | >gi\|2654372\|emb\|CAA82263.1\| (Z28627) protein |
| | | | | | | | phosphatase 1 [Acetabularia cliftonii] |
| 1576 | LIB3602-063-Q1-K6-C10 | 1346750 | BLASTX | 705 | 2.00E−74 | 88 | SERINE/THREONINE PROTEIN |
| | | | | | | | PHOSPHATASE PP1 ISOZYME 1 |
| | | | | | | | >gi\|2654372\|emb\|CAA82263.1\| (Z28627) protein |
| | | | | | | | phosphatase 1 [Acetabularia cliftonii] |
| 1577 | LIB3602-090-Q6-K6-H5 | 5080820 | BLASTX | 346 | 2.00E−32 | 35 | (AC007258) Putative ABC transporter [Arabidopsis |
| | | | | | | | thaliana] |
| 1578 | LIB3602-114-Q1-K1-H5 | 5080820 | BLASTX | 346 | 3.00E−32 | 35 | (AC007258) Putative ABC transporter [Arabidopsis |
| | | | | | | | thaliana] |
| 1579 | LIB3602-040-Q6-K1-F5 | 5080820 | BLASTX | 209 | 2.00E−16 | 37 | (AC007258) Putative ABC transporter [Arabidopsis |
| | | | | | | | thaliana] |
| 1580 | LIB3602-036-Q6-K1-A9 | 3334123 | BLASTX | 274 | 1.00E−37 | 39 | ATP SYNTHASE GAMMA CHAIN, |
| | | | | | | | MITOCHONDRIAL PRECURSOR |
| | | | | | | | >gi\|1655480\|dbj\|BAA13599.1\| (D88374) gamma |
| | | | | | | | subunit of mitochondrial F1-ATPase [Arabidopsis |
| | | | | | | | thaliana] >gi\|2924787\|gb\|AAC04916.1\| |
| | | | | | | | (AC002334) mitochondrial F1-ATPase, gamma |
| | | | | | | | subunit (ATP3_ARATH) [Arabidopsis t |
| 1581 | LIB3602-030-Q6-K1-H11 | 3334123 | BLASTX | 304 | 2.00E−27 | 42 | ATP SYNTHASE GAMMA CHAIN, |
| | | | | | | | MITOCHONDRIAL PRECURSOR |
| | | | | | | | >gi\|1655480\|dbj\|BAA13599.1\| (D88374) gamma |
| | | | | | | | subunit of mitochondrial F1-ATPase [Arabidopsis |
| | | | | | | | thaliana] >gi\|2924787\|gb\|AAC04916.1\| |
| | | | | | | | (AC002334) mitochondrial F1-ATPase, gamma |
| | | | | | | | subunit (ATP3_ARATH) [Arabidopsis t |
| 1582 | LIB3602-120-Q1-K1-B7 | 3334123 | BLASTX | 178 | 1.00E−12 | 34 | ATP SYNTHASE GAMMA CHAIN, |
| | | | | | | | MITOCHONDRIAL PRECURSOR |
| | | | | | | | >gi\|1655480\|dbj\|BAA13599.1\| (D88374) gamma |
| | | | | | | | subunit of mitochondrial F1-ATPase [Arabidopsis |
| | | | | | | | thaliana] >gi\|2924787\|gb\|AAC04916.1\| |
| | | | | | | | (AC002334) mitochondrial F1-ATPase, gamma |
| | | | | | | | subunit (ATP3_ARATH) [Arabidopsis t |
| 1583 | LIB3602-019-Q6-K1-F5 | 133883 | BLASTX | 445 | 3.00E−44 | 85 | 40S RIBOSOMAL PROTEIN S20 (S22) |
| | | | | | | | >gi\|85846\|pir\|\|A37974 ribosomal protein S20, |
| | | | | | | | cytosolic - African clawed frog |
| | | | | | | | >gi\|214758\|gb\|AAA49953.1\| (M34706) ribosomal |
| | | | | | | | protein S22, 40S subunit [Xenopus laevis] |
| 1584 | LIB3602-004-Q1-K1-G10 | 133883 | BLASTX | 255 | 3.00E−32 | 70 | 40S RIBOSOMAL PROTEIN S20 (S22) |
| | | | | | | | >gi\|85846\|pir\|\|A37974 ribosomal protein S20, |
| | | | | | | | cytosolic - African clawed frog |
| | | | | | | | >gi\|214758\|gb\|AAA49953.1\| (M34706) ribosomal |
| | | | | | | | protein S22, 40S subunit [Xenopus laevis] |
| 1585 | LIB3602-114-Q1-K1-D4 | 133883 | BLASTX | 169 | 3.00E−12 | 91 | 40S RIBOSOMAL PROTEIN S20 (S22) |
| | | | | | | | >gi\|85846\|pir\|\|A37974 ribosomal protein S20, |
| | | | | | | | cytosolic - African clawed frog |
| | | | | | | | >gi\|214758\|gb\|AAA49953.1\| (M34706) ribosomal |
| | | | | | | | protein S22, 40S subunit [Xenopus laevis] |
| 1586 | LIB3602-003-Q1-K1-B11 | 7573429 | BLASTX | 244 | 2.00E−20 | 43 | (AL163816) putative protein [Arabidopsis thaliana] |
| 1587 | LIB3602-056-Q6-K1-C9 | 7573429 | BLASTX | 169 | 1.00E−11 | 40 | (AL163816) putative protein [Arabidopsis thaliana] |
| 1588 | LIB3602-003-Q1-K1-B5 | 7573429 | BLASTX | 152 | 1.00E−09 | 39 | (AL163816) putative protein [Arabidopsis thaliana] |
| 1589 | LIB3602-026-Q6-K1-B10 | 5758304 | BLASTX | 479 | 6.00E−48 | 77 | (AF161704) 40S ribosomal protein S17 |
| | | | | | | | [Lycopersicon esculentum] |
| 1590 | LIB3602-105-Q1-K1-H7 | 5758304 | BLASTX | 419 | 7.00E−41 | 73 | (AF161704) 40S ribosomal protein S17 |
| | | | | | | | [Lycopersicon esculentum] |
| 1591 | LIB3602-065-Q1-K6-D4 | 5758304 | BLASTX | 325 | 6.00E−30 | 77 | (AF161704) 40S ribosomal protein S17 |
| | | | | | | | [Lycopersicon esculentum] |
| 1592 | LIB3602-002-P1-K6-H6 | 7447940 | BLASTX | 289 | 1.00E−25 | 47 | hypothetical protein slr1503 - Synechocystis sp. |
| | | | | | | | (strain PCC 6803) >gi\|1653089\|dbj\|BAA18005.1\| |
| | | | | | | | (D90911) hypothetical protein [Synechocystis sp.] |
| 1593 | LIB3602-025-Q6-K1-F8 | 7447940 | BLASTX | 189 | 5.00E−14 | 44 | hypothetical protein slr1503 - Synechocystis sp. |
| | | | | | | | (strain PCC 6803) >gi\|1653089\|dbj\|BAA18005.1\| |
| | | | | | | | (D90911) hypothetical protein [Synechocystis sp.] |
| 1594 | LIB3602-047-Q6-K1-H5 | 7447940 | BLASTX | 167 | 8.00E−12 | 58 | hypothetical protein slr1503 - Synechocystis sp. |
| | | | | | | | (strain PCC 6803) >gi\|1653089\|dbj\|BAA18005.1\| |
| | | | | | | | (D90911) hypothetical protein [Synechocystis sp.] |
| 1595 | LIB3602-054-Q6-K1-D5 | 6015437 | BLASTN | 38 | 1.00E−12 | 100 | Homo sapiens PEX1 mRNA, complete cds |
| 1596 | LIB3602-054-Q6-K1-E5 | 6015437 | BLASTN | 37 | 3.00E−11 | 100 | Homo sapiens PEX1 mRNA, complete cds |
| 1597 | LIB3602-052-Q6-K1-B7 | 6015437 | BLASTN | 37 | 5.00E−11 | 100 | Homo sapiens PEX1 mRNA, complete cds |
| 1598 | LIB3602-037-Q6-K1-D2 | 5360595 | BLASTX | 301 | 4.00E−27 | 42 | (AB022330) nClpP5 [Arabidopsis thaliana] |
| 1599 | LIB3602-046-Q6-K1-H11 | 5360595 | BLASTX | 259 | 3.00E−22 | 61 | (AB022330) nClpP5 [Arabidopsis thaliana] |
| 1600 | LIB3602-046-Q6-K1-H12 | 5360595 | BLASTX | 255 | 9.00E−22 | 60 | (AB022330) nClpP5 [Arabidopsis thaliana] |
| 1601 | LIB3602-041-Q6-K1-G8 | 543696 | BLASTX | 656 | 1.00E−68 | 63 | translation elongation factor EF-3 homolog - |
| | | | | | | | Chlorella virus CVK2 >gi\|454301\|dbj\|BAA03956.1\| |
| | | | | | | | (D16505) translation elongation factor-3 [Chlorella |
| | | | | | | | virus] |
| 1602 | LIB3602-073-Q1-K1-F2 | 543696 | BLASTX | 488 | 8.00E−58 | 61 | translation elongation factor EF-3 homolog - |
| | | | | | | | Chlorella virus CVK2 >gi\|454301\|dbj\|BAA03956.1\| |
| | | | | | | | (D16505) translation elongation factor-3 [Chlorella |
| | | | | | | | virus] |
| 1603 | LIB3602-095-Q6-K6-A1 | 543696 | BLASTX | 244 | 8.00E−21 | 55 | translation elongation factor EF-3 homolog - |
| | | | | | | | Chlorella virus CVK2 >gi\|454301\|dbj\|BAA03956.1\| |
| | | | | | | | (D16505) translation elongation factor-3 [Chlorella |
| | | | | | | | virus] |
| 1604 | LIB3602-015-Q6-K1-D10 | 7499991 | BLASTX | 277 | 3.00E−24 | 35 | hypothetical protein F27C1.7 - Caenorhabditis |
| | | | | | | | elegans >gi\|1703605\|gb\|AAB37654.1\| (U80441) |
| | | | | | | | coded for by C. elegans cDNA yk63g10.5; coded |
| | | | | | | | for by C. elegans cDNA yk105c10.5; coded for by |
| | | | | | | | C. elegans cDNA yk167c12.5; coded for by C. elegans |
| | | | | | | | cDNA yk167c12.3; co |
| 1605 | LIB3602-050-Q6-K1-H6 | 7499991 | BLASTX | 234 | 2.00E−19 | 40 | hypothetical protein F27C1.7 - Caenorhabditis |
| | | | | | | | elegans >gi\|1703605\|gb\|AAB37654.1\| (U80441) |
| | | | | | | | coded for by C. elegans cDNA yk63g10.5; coded |
| | | | | | | | for by C. elegans cDNA yk105c10.5; coded for by |
| | | | | | | | C. elegans cDNA yk167c12.5; coded for by C. elegans |
| | | | | | | | cDNA yk167c12.3; co |
| 1606 | LIB3602-074-Q1-K1-E12 | 7499991 | BLASTX | 214 | 8.00E−17 | 38 | hypothetical protein F27C1.7 - Caenorhabditis |
| | | | | | | | elegans >gi\|1703605\|gb\|AAB37654.1\| (U80441) |
| | | | | | | | coded for by C. elegans cDNA yk63g10.5; coded |
| | | | | | | | for by C. elegans cDNA yk105c10.5; coded for by |
| | | | | | | | C. elegans cDNA yk167c12.5; coded for by C. elegans |
| | | | | | | | cDNA yk167c12.3; co |
| 1607 | LIB3602-053-Q6-K1-D5 | 1245180 | BLASTX | 415 | 2.00E−40 | 47 | (U49236) arginine deiminase [Giardia intestinalis] |
| 1608 | LIB3602-053-Q6-K1-D11 | 1245180 | BLASTX | 413 | 4.00E−40 | 47 | (U49236) arginine deiminase [Giardia intestinalis] |
| 1609 | LIB3602-037-Q6-K1-H9 | 1245180 | BLASTX | 199 | 3.00E−15 | 45 | (U49236) arginine deiminase [Giardia intestinalis] |
| 1610 | LIB3602-027-Q6-K1-F3 | 730449 | BLASTX | 390 | 2.00E−37 | 59 | 60S RIBOSOMAL PROTEIN L13-1 (COLD |
| | | | | | | | INDUCED PROTEIN C24A) |
| | | | | | | | >gi\|480647\|pir\|\|S37132 ribosomal protein L13.A - |
| | | | | | | | rape >gi\|398918\|emb\|CAA80341.1\| (Z22618) cold |
| | | | | | | | induced protein (BnC24A) [Brassica napus] |
| 1611 | LIB3602-009-Q6-K1-A2 | 730449 | BLASTX | 342 | 6.00E−32 | 55 | 60S RIBOSOMAL PROTEIN L13-1 (COLD |
| | | | | | | | INDUCED PROTEIN C24A) |
| | | | | | | | >gi\|480647\|pir\|\|S37132 ribosomal protein L13.A - |
| | | | | | | | rape >gi\|398918\|emb\|CAA80341.1\| (Z22618) cold |
| | | | | | | | induced protein (BnC24A) [Brassica napus] |
| 1612 | LIB3602-066-Q1-K6-D2 | 730449 | BLASTX | 296 | 1.00E−26 | 54 | 60S RIBOSOMAL PROTEIN L13-1 (COLD |
| | | | | | | | INDUCED PROTEIN C24A) |
| | | | | | | | >gi\|480647\|pir\|\|S37132 ribosomal protein L13.A - |
| | | | | | | | rape >gi\|398918\|emb\|CAA80341.1\| (Z22618) cold |
| | | | | | | | induced protein (BnC24A) [Brassica napus] |
| 1613 | LIB3602-045-Q6-K1-B12 | 6552397 | BLASTX | 187 | 8.00E−14 | 73 | (AB030493) thiamin biosynthetic enzyme [Glycine |
| | | | | | | | max] |
| 1614 | LIB3602-005-Q1-K1-G9 | 6552397 | BLASTX | 184 | 2.00E−13 | 68 | (AB030493) thiamin biosynthetic enzyme [Glycine |
| | | | | | | | max] |
| 1615 | LIB3602-050-Q6-K1-G8 | 6552397 | BLASTX | 183 | 2.00E−13 | 76 | (AB030493) thiamin biosynthetic enzyme [Glycine |
| | | | | | | | max] |
| 1616 | LIB3602-076-Q6-K6-A3 | 6714365 | BLASTX | 183 | 3.00E−13 | 47 | (AC012562) unknown protein [Arabidopsis |
| | | | | | | | thaliana] |
| 1617 | LIB3602-069-Q1-K1-B1 | 6714365 | BLASTX | 181 | 6.00E−13 | 47 | (AC012562) unknown protein [Arabidopsis |
| | | | | | | | thaliana] |
| 1618 | LIB3602-003-Q1-K1-A4 | 6714365 | BLASTX | 157 | 3.00E−10 | 50 | (AC012562) unknown protein [Arabidopsis |
| | | | | | | | thaliana] |
| 1619 | LIB3602-089-Q6-K6-G9 | 3293539 | BLASTX | 469 | 9.00E−47 | 60 | (AF072709) unknown [Streptomyces lividans] |
| 1620 | LIB3602-095-Q6-K6-G9 | 3293539 | BLASTX | 469 | 1.00E−46 | 60 | (AF072709) unknown [Streptomyces lividans] |
| 1621 | LIB3602-112-Q1-K1-G9 | 3293539 | BLASTX | 469 | 1.00E−46 | 60 | (AF072709) unknown [Streptomyces lividans] |
| 1622 | LIB3602-088-Q6-K6-H3 | 6456166 | BLASTX | 549 | 4.00E−56 | 74 | (AC011622) GMP synthase [Arabidopsis thaliana] |
| 1623 | LIB3602-104-Q1-K1-E6 | 6456166 | BLASTX | 548 | 5.00E−56 | 73 | (AC011622) GMP synthase [Arabidopsis thaliana] |
| 1624 | LIB3602-061-Q6-K1-H12 | 6456166 | BLASTX | 455 | 3.00E−45 | 75 | (AC011622) GMP synthase [Arabidopsis thaliana] |
| 1625 | LIB3602-111-Q1-K1-C10 | 7447845 | BLASTX | 553 | 9.00E−57 | 76 | hypothetical protein T16L1.170 - Arabidopsis |
| | | | | | | | thaliana >gi\|3549670\|emb\|CAA20581.1\| |
| | | | | | | | (AL031394) putative protein [Arabidopsis thaliana] |
| | | | | | | | >gi\|7270317\|emb\|CAB80085.1\| (AL161584) |
| | | | | | | | putative protein [Arabidopsis thaliana] |
| 1626 | LIB3602-006-Q1-K1-B10 | 7447845 | BLASTX | 362 | 3.00E−34 | 73 | hypothetical protein T16L1.170 - Arabidopsis |
| | | | | | | | thaliana >gi\|3549670\|emb\|CAA20581.1\| |
| | | | | | | | (AL031394) putative protein [Arabidopsis thaliana] |
| | | | | | | | >gi\|7270317\|emb\|CAB80085.1\| (AL161584) |
| | | | | | | | putative protein [Arabidopsis thaliana] |
| 1627 | LIB3602-043-Q6-K1-E11 | 7447845 | BLASTX | 299 | 4.00E−27 | 57 | hypothetical protein T16L1.170 - Arabidopsis |
| | | | | | | | thaliana >gi\|3549670\|emb\|CAA20581.1\| |
| | | | | | | | (AL031394) putative protein [Arabidopsis thaliana] |
| | | | | | | | >gi\|7270317\|emb\|CAB80085.1\| (AL161584) |
| | | | | | | | putative protein [Arabidopsis thaliana] |
| 1628 | LIB3602-066-Q1-K6-C3 | 7019650 | BLASTX | 278 | 2.00E−24 | 63 | (AL132954) ribosomal protein L35-like |
| | | | | | | | [Arabidopsis thaliana] |
| 1629 | LIB3602-117-Q1-K1-C8 | 7019650 | BLASTX | 157 | 9.00E−13 | 63 | (AL132954) ribosomal protein L35-like |
| | | | | | | | [Arabidopsis thaliana] |
| 1630 | LIB3602-044-Q6-K1-D10 | 7019650 | BLASTX | 145 | 1.00E−10 | 51 | (AL132954) ribosomal protein L35-like |
| | | | | | | | [Arabidopsis thaliana] |
| 1631 | LIB3602-044-Q6-K1-H9 | 2501354 | BLASTX | 509 | 1.00E−51 | 65 | TRANSKETOLASE 7 (TK) |
| | | | | | | | >gi\|1084441\|pir\|\|S54301 transketolase (EC 2.2.1.1) |
| | | | | | | | 7 - Craterostigma plantagineum |
| | | | | | | | >gi\|664903\|emb\|CAA86609.1\| (Z46648) |
| | | | | | | | transketolase [Craterostigma plantagineum] |
| 1632 | LIB3602-055-Q6-K1-H2 | 2501354 | BLASTX | 479 | 5.00E−48 | 61 | TRANSKETOLASE 7 (TK) |
| | | | | | | | >gi\|1084441\|pir\|\|S54301 transketolase (EC 2.2.1.1) |
| | | | | | | | 7 - Craterostigma plantagineum |
| | | | | | | | >gi\|664903\|emb\|CAA86609.1\| (Z46648) |
| | | | | | | | transketolase [Craterostigma plantagineum] |
| 1633 | LIB3602-044-Q6-K1-H10 | 2501354 | BLASTX | 219 | 1.00E−17 | 45 | TRANSKETOLASE 7 (TK) |
| | | | | | | | >gi\|1084441\|pir\|\|S54301 transketolase (EC 2.2.1.1) |
| | | | | | | | 7 - Craterostigma plantagineum |
| | | | | | | | >gi\|664903\|emb\|CAA86609.1\| (Z46648) |
| | | | | | | | transketolase [Craterostigma plantagineum] |
| 1634 | LIB3602-030-Q6-K1-G6 | 7023084 | BLASTX | 183 | 2.00E−13 | 35 | (AK001677) unnamed protein product [Homo |
| | | | | | | | sapiens] |
| 1635 | LIB3602-093-Q6-K6-C6 | 7023084 | BLASTX | 161 | 1.00E−10 | 37 | (AK001677) unnamed protein product [Homo |
| | | | | | | | sapiens] |
| 1636 | LIB3602-113-Q1-K1-G1 | 7023084 | BLASTX | 161 | 1.00E−10 | 37 | (AK001677) unnamed protein product [Homo |
| | | | | | | | sapiens] |
| 1637 | LIB3602-109-Q1-K1-E10 | 7485750 | BLASTX | 413 | 3.00E−40 | 52 | hypothetical protein F19B15.90 - Arabidopsis |
| | | | | | | | thaliana >gi\|4972052\|emb\|CAB43920.1\| |
| | | | | | | | (AL078470) putative protein [Arabidopsis thaliana] |
| | | | | | | | >gi\|7269804\|emb\|CAB79664.1\| (AL161574) |
| | | | | | | | putative protein [Arabidopsis thaliana] |
| 1638 | LIB3602-078-Q6-K6-B7 | 7485750 | BLASTX | 269 | 3.00E−23 | 60 | hypothetical protein F19B15.90 - Arabidopsis |
| | | | | | | | thaliana >gi\|4972052\|emb\|CAB43920.1\| |
| | | | | | | | (AL078470) putative protein [Arabidopsis thaliana] |
| | | | | | | | >gi\|7269804\|emb\|CAB79664.1\| (AL161574) |
| | | | | | | | putative protein [Arabidopsis thaliana] |
| 1639 | LIB3602-068-Q1-K1-E7 | 7485750 | BLASTX | 263 | 1.00E−22 | 60 | hypothetical protein F19B15.90 - Arabidopsis |
| | | | | | | | thaliana >gi\|4972052\|emb\|CAB43920.1\| |
| | | | | | | | (AL078470) putative protein [Arabidopsis thaliana] |
| | | | | | | | >gi\|7269804\|emb\|CAB79664.1\| (AL161574) |
| | | | | | | | putative protein [Arabidopsis thaliana] |
| 1640 | LIB3602-094-Q6-K6-E12 | 1902882 | BLASTX | 202 | 1.00E−15 | 47 | (AB001833) ribosomal protein YL16 homolog |
| | | | | | | | [Schizosaccharomyces pombe] |
| 1641 | LIB3602-091-Q6-K6-E12 | 1902882 | BLASTX | 193 | 2.00E−14 | 50 | (AB001833) ribosomal protein YL16 homolog |
| | | | | | | | [Schizosaccharomyces pombe] |
| 1642 | LIB3602-040-Q6-K1-A2 | 1902882 | BLASTX | 148 | 1.00E−09 | 60 | (AB001833) ribosomal protein YL16 homolog |
| | | | | | | | [Schizosaccharomyces pombe] |
| 1643 | LIB3602-115-Q1-K1-C1 | 7441176 | BLASTX | 330 | 1.00E−30 | 60 | ribosomal protein L35, cytosolic - Arabidopsis |
| | | | | | | | thaliana >gi\|3355468\|gb\|AAC27830.1\| (AC004218) |
| | | | | | | | 60S ribosomal protein L35 [Arabidopsis thaliana] |
| 1644 | LIB3602-013-Q6-K1-D1 | 7441176 | BLASTX | 238 | 3.00E−20 | 59 | ribosomal protein L35, cytosolic - Arabidopsis |
| | | | | | | | thaliana >gi\|3355468\|gb\|AAC27830.1\| (AC004218) |
| | | | | | | | 60S ribosomal protein L35 [Arabidopsis thaliana] |
| 1645 | LIB3602-070-Q1-K1-E5 | 7441176 | BLASTX | 235 | 8.00E−20 | 59 | ribosomal protein L35, cytosolic - Arabidopsis |
| | | | | | | | thaliana >gi\|3355468\|gb\|AAC27830.1\| (AC004218) |
| | | | | | | | 60S ribosomal protein L35 [Arabidopsis thaliana] |
| 1646 | LIB3602-108-Q1-K1-G1 | 3885884 | BLASTX | 340 | 6.00E−32 | 65 | (AF093630) 60S ribosomal protein L21 [Oryza |
| | | | | | | | sativa] |
| 1647 | LIB3602-106-Q1-K1-B3 | 3885884 | BLASTX | 223 | 6.00E−18 | 41 | (AF093630) 60S ribosomal protein L21 [Oryza |
| | | | | | | | sativa] |
| 1648 | LIB3602-051-Q6-K1-D12 | 3885884 | BLASTX | 150 | 5.00E−10 | 47 | (AF093630) 60S ribosomal protein L21 [Oryza |
| | | | | | | | sativa] |
| 1649 | LIB3602-020-Q6-K1-B9 | 2851583 | BLASTX | 242 | 3.00E−20 | 58 | ALPHA-AMYLASE TYPE B ISOZYME |
| | | | | | | | PRECURSOR (1,4-ALPHA-D-GLUCAN |
| | | | | | | | GLUCANOHYDROLASE) (AMY2-2) (HIGH PI |
| | | | | | | | ALPHA-AMYLASE) >gi\|1070490\|pir\|\|ALBHB |
| | | | | | | | alpha-amylase (EC 3.2.1.1) B precursor 6-4 - barley |
| | | | | | | | >gi\|166995\|gb\|AA98790.1\| (K02637) Hordeum |
| | | | | | | | vulgare alpha-amylase type |
| 1650 | LIB3602-074-Q1-K1-D2 | 2851583 | BLASTX | 212 | 1.00E−16 | 54 | ALPHA-AMYLASE TYPE B ISOZYME |
| | | | | | | | PRECURSOR (1,4-ALPHA-D-GLUCAN |
| | | | | | | | GLUCANOHYDROLASE) (AMY2-2) (HIGH PI |
| | | | | | | | ALPHA-AMYLASE) >gi\|1070490\|pir\|\|ALBHB |
| | | | | | | | alpha-amylase (EC 3.2.1.1) B precursor 6-4 - barley |
| | | | | | | | >gi\|166995\|gb\|AAA98790.1\| (K02637) Hordeum |
| | | | | | | | vulgare alpha-amylase type |
| 1651 | LIB3602-032-Q6-K1-A10 | 2851583 | BLASTX | 189 | 4.00E−14 | 56 | ALPHA-AMYLASE TYPE B ISOZYME |
| | | | | | | | PRECURSOR (1,4-ALPHA-D-GLUCAN |
| | | | | | | | GLUCANOHYDROLASE) (AMY2-2) (HIGH PI |
| | | | | | | | ALPHA-AMYLASE) >gi\|1070490\|pir\|\|ALBHB |
| | | | | | | | alpha-amylase (EC 3.2.1.1) B precursor 6-4 - barley |
| | | | | | | | >gi\|166995\|gb\|AAA98790.1\| (K02637) Hordeum |
| | | | | | | | vulgare alpha-amylase type |
| 1652 | LIB3602-087-Q6-K1-A4 | 132167 | BLASTX | 795 | 5.00E−85 | 87 | RIBULOSE BISPHOSPHATE |
| | | | | | | | CARBOXYLASE/OXYGENASE ACTIVASE, |
| | | | | | | | CHLOROPLAST PRECURSOR (RUBISCO |
| | | | | | | | ACTIVASE) >gi\|320539\|pir\|\|A45507 ribulose- |
| | | | | | | | bisphosphate carboxylase activase (EC 6.3.4.—) |
| | | | | | | | precursor - Chlamydomonas reinhardtii |
| | | | | | | | >gi\|167434\|gb\|AAA33091.1\| (M62962) ribulose |
| 1653 | LIB3602-084-Q6-K1-A4 | 132167 | BLASTX | 774 | 2.00E−82 | 81 | RIBULOSE BISPHOSPHATE |
| | | | | | | | CARBOXYLASE/OXYGENASE ACTIVASE, |
| | | | | | | | CHLOROPLAST PRECURSOR (RUBISCO |
| | | | | | | | ACTIVASE) >gi\|320539\|pir\|\|A45507 ribulose- |
| | | | | | | | bisphosphate carboxylase activase (EC 6.3.4.—) |
| | | | | | | | precursor - Chlamydomonas reinhardtii |
| | | | | | | | >gi\|167434\|gb\|AAA33091.1\| (M62962) ribulose |
| 1654 | LIB3602-050-Q6-K1-C1 | 132167 | BLASTX | 760 | 5.00E−81 | 96 | RIBULOSE BISPHOSPHATE |
| | | | | | | | CARBOXYLASE/OXYGENASE ACTIVASE, |
| | | | | | | | CHLOROPLAST PRECURSOR (RUBISCO |
| | | | | | | | ACTIVASE) >gi\|320539\|pir\|\|A45507 ribulose- |
| | | | | | | | bisphosphate carboxylase activase (EC 6.3.4.—) |
| | | | | | | | precursor - Chlamydomonas reinhardtii |
| | | | | | | | >gi\|167434\|gb\|AAA33091.1\| (M62962) ribulose |
| 1655 | LIB3602-095-Q6-K6-A5 | 117971 | BLASTX | 429 | 5.00E−42 | 79 | CYTOCHROME C >gi\|322374\|pir\|\|S29514 |
| | | | | | | | cytochrome c - Chlamydomonas reinhardtii |
| | | | | | | | >gi\|167413\|gb\|AAA33084.1\| (M35173) |
| | | | | | | | apocytochrome c (cyc) [Chlamydomonas |
| | | | | | | | reinhardtii ] >gi\|2660500\|emb\|CAB16954.1\| |
| | | | | | | | (Z99829) cytochrome c [Chlamydomonas |
| | | | | | | | reinhardtii] >gi\|226382\|prf\| |
| 1656 | LIB3602-117-Q1-K1-D12 | 117971 | BLASTX | 396 | 2.00E−38 | 82 | CYTOCHROME C >gi\|322374\|pir\|\|S29514 |
| | | | | | | | cytochrome c - Chlamydomonas reinhardtii |
| | | | | | | | >gi\|167413\|gb\|AAA33084.1\| (M35173) |
| | | | | | | | apocytochrome c (cyc) [Chlamydomonas |
| | | | | | | | reinhardtii] >gi\|2660500\|emb\|CAB16954.1\| |
| | | | | | | | (Z99829) cytochrome c [Chlamydomonas |
| | | | | | | | reinhardtii] >gi\|226382\|prf\| |
| 1657 | LIB3602-013-Q6-K1-C1 | 117971 | BLASTX | 246 | 1.00E−20 | 85 | CYTOCHROME C >gi\|322374\|pir\|\|S29514 |
| | | | | | | | cytochrome c - Chlamydomonas reinhardtii |
| | | | | | | | >gi\|167413\|gb\|AAA33084.1\| (M35173) |
| | | | | | | | apocytochrome c (cyc) [Chlamydomonas |
| | | | | | | | reinhardtii] >gi\|2660500\|emb\|CAB16954.1\| |
| | | | | | | | (Z99829) cytochrome c [Chlamydomonas |
| | | | | | | | reinhardtii] >gi\|226382\|prf\| |
| 1658 | LIB3602-014-Q6-K1-H9 | 3913425 | BLASTX | 387 | 3.00E−37 | 69 | PUTATIVE PRE-MRNA SPLICING FACTOR |
| | | | | | | | ATP-DEPENDENT RNA HELICASE |
| | | | | | | | >gi\|2275203\|gb\|AAB63825.1\| (AC002337) putative |
| | | | | | | | pre-mRNA splicing factor RNA helicase |
| | | | | | | | [Arabidopsis thaliana] |
| 1659 | LIB3602-035-Q1-K1-G10 | 3913425 | BLASTX | 371 | 2.00E−35 | 67 | PUTATIVE PRE-MRNA SPLICING FACTOR |
| | | | | | | | ATP-DEPENDENT RNA HELICASE |
| | | | | | | | >gi\|2275203\|gb\|AAB63825.1\| (AC002337) putative |
| | | | | | | | pre-mRNA splicing factor RNA helicase |
| | | | | | | | [Arabidopsis thaliana] |
| 1660 | LIB3602-034-Q6-K1-G10 | 3913425 | BLASTX | 312 | 1.00E−28 | 63 | PUTATIVE PRE-MRNA SPLICING FACTOR |
| | | | | | | | ATP-DEPENDENT RNA HELICASE |
| | | | | | | | >gi\|2275203\|gb\|AAB63825.1\| (AC002337) putative |
| | | | | | | | pre-mRNA splicing factor RNA helicase |
| | | | | | | | [Arabidopsis thaliana] |
| 1661 | LIB3602-118-Q1-K1-G11 | 167427 | BLASTN | 46 | 1.00E−16 | 90 | C. reinhardtii oxygen-evolving enhancer protein 2 |
| | | | | | | | (OEE2) mRNA, complete cds |
| 1662 | LIB3602-094-Q6-K6-F12 | 167427 | BLASTN | 46 | 2.00E−16 | 90 | C. reinhardtii oxygen-evolving enhancer protein 2 |
| | | | | | | | (OEE2) mRNA, complete cds |
| 1663 | LIB3602-114-Q1-K1-D11 | 167427 | BLASTN | 46 | 3.00E−16 | 90 | C. reinhardtii oxygen-evolving enhancer protein 2 |
| | | | | | | | (OEE2) mRNA, complete cds |
| 1664 | LIB3602-109-Q1-K1-G1 | 7487122 | BLASTX | 265 | 7.00E−25 | 43 | hypothetical protein T17F15.230 - Arabidopsis |
| | | | | | | | thaliana >gi\|4678338\|emb\|CAB41149.1\| |
| | | | | | | | (AL049658) putative protein [Arabidopsis thaliana] |
| 1665 | LIB3602-058-Q6-K1-B11 | 7487122 | BLASTX | 197 | 5.00E−15 | 41 | hypothetical protein T17F15.230 - Arabidopsis |
| | | | | | | | thaliana >gi\|4678338\|emb\|CAB41149.1\| |
| | | | | | | | (AL049658) putative protein [Arabidopsis thaliana] |
| 1666 | LIB3602-012-Q6-K1-B10 | 7487122 | BLASTX | 198 | 6.00E−15 | 28 | hypothetical protein T17F15.230 - Arabidopsis |
| | | | | | | | thaliana >gi\|4678338\|emb\|CAB41149.1\| |
| | | | | | | | (AL049658) putative protein [Arabidopsis thaliana] |
| 1667 | LIB3602-076-Q6-K6-C2 | 6752882 | BLASTX | 317 | 6.00E−29 | 53 | (AF220200) nascent polypeptide associated |
| | | | | | | | complex alpha chain [Pinus taeda] |
| 1668 | LIB3602-105-Q1-K1-H4 | 6752882 | BLASTX | 309 | 5.00E−28 | 52 | (AF220200) nascent polypeptide associated |
| | | | | | | | complex alpha chain [Pinus taeda] |
| 1669 | LIB3602-050-Q6-K1-A5 | 6752882 | BLASTX | 280 | 1.00E−24 | 78 | (AF220200) nascent polypeptide associated |
| | | | | | | | complex alpha chain [Pinus taeda] |
| 1670 | LIB3602-023-Q6-K1-E2 | 4107276 | BLASTX | 440 | 5.00E−47 | 66 | (X98506) acetyl-CoA synthetase [Solanum |
| | | | | | | | tuberosum] |
| 1671 | LIB3602-089-Q6-K6-F4 | 4107276 | BLASTX | 179 | 9.00E−13 | 69 | (X98506) acetyl-CoA synthetase [Solanum |
| | | | | | | | tuberosum] |
| 1672 | LIB3602-056-Q6-K1-F3 | 4107276 | BLASTX | 178 | 9.00E−13 | 76 | (X98506) acetyl-CoA synthetase [Solanum |
| | | | | | | | tuberosum] |
| 1673 | LIB3602-060-Q6-K1-C7 | 421985 | BLASTX | 311 | 2.00E−28 | 79 | ribosomal protein L17.1, cytosolic - barley |
| 1674 | LIB3602-015-Q6-K1-C3 | 421985 | BLASTX | 275 | 3.00E−24 | 79 | ribosomal protein L17.1, cytosolic - barley |
| 1675 | LIB3602-036-Q6-K1-B8 | 421985 | BLASTX | 245 | 1.00E−20 | 76 | ribosomal protein L17.1, cytosolic - barley |
| 1676 | LIB3602-003-Q1-K1-C8 | 5019305 | BLASTX | 252 | 2.00E−21 | 33 | (AJ011563) carnitine/acyl carnitine carrier |
| | | | | | | | [Emericella nidulans] |
| 1677 | LIB3602-039-Q6-K1-C7 | 5019305 | BLASTX | 251 | 3.00E−21 | 35 | (AJ011563) carnitine/acyl carnitine carrier |
| | | | | | | | [Emericella nidulans] |
| 1678 | LIB3602-003-Q1-K1-C9 | 5019305 | BLASTX | 178 | 9.00E−13 | 33 | (AJ011563) carnitine/acyl carnitine carrier |
| | | | | | | | [Emericella nidulans] |
| 1679 | LIB3602-034-Q6-K1-C6 | 5174741 | BLASTX | 394 | 5.00E−38 | 52 | ubiquitin carboxyl-terminal esterase L3 (ubiquitin |
| | | | | | | | thiolesterase) |
| | | | | | | | >gi\|136682\|sp\|P15374\|UBL3_HUMAN |
| | | | | | | | UBIQUITIN CARBOXYL-TERMINAL |
| | | | | | | | HYDROLASE ISOZYME L3 (UCH-L3) |
| | | | | | | | (UBIQUITIN THIOLESTERASE L3) |
| | | | | | | | >gi\|108014\|pir\|\|A40085 ubiquitin thiolesterase (EC |
| | | | | | | | 3.1.2.15) L3 - huma |
| 1680 | LIB3602-104-Q1-K1-D11 | 5174741 | BLASTX | 393 | 8.00E−38 | 52 | ubiquitin carboxyl-terminal esterase L3 (ubiquitin |
| | | | | | | | thiolesterase) |
| | | | | | | | >gi\|136682\|sp\|P15374\|UBL3_HUMAN |
| | | | | | | | UBIQUITIN CARBOXYL-TERMINAL |
| | | | | | | | HYDROLASE ISOZYME L3 (UCH-L3) |
| | | | | | | | (UBIQUITIN THIOLESTERASE L3) |
| | | | | | | | >gi\|108014\|pir\|\|A40085 ubiquitin thiolesterase (EC |
| | | | | | | | 3.1.2.15) L3 - huma |
| 1681 | LIB3602-035-Q1-K1-C6 | 5174741 | BLASTX | 356 | 1.00E−33 | 54 | ubiquitin carboxyl-terminal esterase L3 (ubiquitin |
| | | | | | | | thiolesterase) |
| | | | | | | | >gi\|136682\|sp\|P15374\|UBL3_HUMAN |
| | | | | | | | UBIQUITIN CARBOXYL-TERMINAL |
| | | | | | | | HYDROLASE ISOZYME L3 (UCH-L3) |
| | | | | | | | (UBIQUITIN THIOLESTERASE L3) |
| | | | | | | | >gi\|108014\|pir\|\|A40085 ubiquitin thiolesterase (EC |
| | | | | | | | 3.1.2.15) L3 - huma |
| 1682 | LIB3602-050-Q6-K1-B11 | 7446976 | BLASTX | 589 | 6.00E−61 | 73 | 1-deoxy-D-xylulose-5-phosphate synthase precursor - |
| | | | | | | | Chlamydomonas reinhardtii |
| | | | | | | | >gi\|4185881\|emb\|CAA07554.1\| (AJ007559) 1- |
| | | | | | | | deoxy-D-xylulose-5-phosphate synthase |
| | | | | | | | [Chlamydomonas reinhardtii] |
| 1683 | LIB3602-109-Q1-K1-E5 | 7446976 | BLASTX | 429 | 1.00E−58 | 69 | 1-deoxy-D-xylulose-5-phosphate synthase precursor - |
| | | | | | | | Chlamydomonas reinhardtii |
| | | | | | | | >gi\|4185881\|emb\|CAA07554.1\| (AJ007559) 1- |
| | | | | | | | deoxy-D-xylulose-5-phosphate synthase |
| | | | | | | | [Chlamydomonas reinhardtii] |
| 1684 | LIB3602-020-Q6-K1-H8 | 7446976 | BLASTX | 521 | 7.00E−53 | 65 | 1-deoxy-D-xylulose-5-phosphate synthase precursor - |
| | | | | | | | Chlamydomonas reinhardtii |
| | | | | | | | >gi\|4185881\|emb\|CAA07554.1\| (AJ007559) 1- |
| | | | | | | | deoxy-D-xylulose-5-phosphate synthase |
| | | | | | | | [Chlamydomonas reinhardtii] |
| 1685 | LIB3602-064-Q1-K6-A2 | 238318 | BLASTX | 578 | 2.00E−59 | 58 | (S62652) branched-chain alpha-keto acid |
| | | | | | | | dehydrogenase E1 alpha subunit [human, Peptide, |
| | | | | | | | 443 aa] [Homo sapiens] |
| 1686 | LIB3602-076-Q6-K6-H5 | 238318 | BLASTX | 544 | 2.00E−55 | 55 | (S62652) branched-chain alpha-keto acid |
| | | | | | | | dehydrogenase E1 alpha subunit [human, Peptide, |
| | | | | | | | 443 aa] [Homo sapiens] |
| 1687 | LIB3602-022-Q6-K1-A10 | 238318 | BLASTX | 177 | 1.00E−12 | 40 | (S62652) branched-chain alpha-keto acid |
| | | | | | | | dehydrogenase E1 alpha subunit [human, Peptide, |
| | | | | | | | 443 aa] [Homo sapiens] |
| 1688 | LIB3602-012-Q6-K1-C3 | 7485188 | BLASTX | 484 | 2.00E−48 | 58 | hypothetical protein - Arabidopsis thaliana |
| | | | | | | | >gi\|2245118\|emb\|CAB10540.1\| (Z97343) |
| | | | | | | | hypothetical protein [Arabidopsis thaliana] |
| | | | | | | | >gi\|7268512\|emb\|CAB78763.1\| (AL161546) |
| | | | | | | | hypothetical protein [Arabidopsis thaliana] |
| 1689 | LIB3602-032-Q6-K1-H9 | 7485188 | BLASTX | 389 | 2.00E−37 | 57 | hypothetical protein - Arabidopsis thaliana |
| | | | | | | | >gi\|2245118\|emb\|CAB10540.1\| (Z97343) |
| | | | | | | | hypothetical protein [Arabidopsis thaliana] |
| | | | | | | | >gi\|7268512\|emb\|CAB78763.1\| (AL161546) |
| | | | | | | | hypothetical protein [Arabidopsis thaliana] |
| 1690 | LIB3602-027-Q6-K1-A10 | 7485188 | BLASTX | 384 | 8.00E−37 | 60 | hypothetical protein - Arabidopsis thaliana |
| | | | | | | | >gi\|2245118\|emb\|CAB10540.1\| (Z97343) |
| | | | | | | | hypothetical protein [Arabidopsis thaliana] |
| | | | | | | | >gi\|7268512\|emb\|CAB78763.1\| (AL161546) |
| | | | | | | | hypothetical protein [Arabidopsis thaliana] |
| 1691 | LIB3602-054-Q6-K1-A2 | 3915826 | BLASTX | 207 | 3.00E−16 | 51 | 60S RIBOSOMAL PROTEIN L5 |
| 1692 | LIB3602-053-Q6-K1-H11 | 3915826 | BLASTX | 159 | 1.00E−10 | 55 | 60S RIBOSOMAL PROTEIN L5 |
| 1693 | LIB3602-052-Q6-K1-C9 | 3915826 | BLASTX | 145 | 6.00E−09 | 53 | 60S RIBOSOMAL PROTEIN L5 |
| 1694 | LIB3602-088-Q6-K6-C5 | 7435750 | BLASTX | 362 | 3.00E−34 | 51 | cell division control protein 48, AAA family |
| | | | | | | | (cdc48-2) homolog - Archaeoglobus fulgidus |
| | | | | | | | >gi\|2648442\|gb\|AAB89157.1\| (AE000959) cell |
| | | | | | | | division control protein 48, AAA family (cdc48-2) |
| | | | | | | | [Archaeoglobus fulgidus] |
| 1695 | LIB3602-118-Q1-K1-C5 | 7435750 | BLASTX | 348 | 1.00E−32 | 52 | cell division control protein 48, AAA family |
| | | | | | | | (cdc48-2) homolog - Archaeoglobus fulgidus |
| | | | | | | | >gi\|2648442\|gb\|AAB89157.1\| (AE000959) cell |
| | | | | | | | division control protein 48, AAA family (cdc48-2) |
| | | | | | | | [Archaeoglobus fulgidus] |
| 1696 | LIB3602-090-Q6-K6-F2 | 7435750 | BLASTX | 331 | 1.00E−30 | 48 | cell division control protein 48, AAA family |
| | | | | | | | (cdc48-2) homolog - Archaeoglobus fulgidus |
| | | | | | | | >gi\|2648442\|gb\|AAB89157.1\| (AE000959) cell |
| | | | | | | | division control protein 48, AAA family (cdc48-2) |
| | | | | | | | [Archaeoglobus fulgidus] |
| 1697 | LIB3602-104-Q1-K1-H9 | 7384996 | BLASTX | 185 | 2.00E−13 | 49 | (AF153422) lacZ [Cloning vector pTG8] |
| 1698 | LIB3602-100-Q1-K1-C11 | 7384996 | BLASTX | 161 | 6.00E−11 | 43 | (AF153422) lacZ [Cloning vector pTG8] |
| 1699 | LIB3602-055-Q6-K1-H7 | 7384996 | BLASTX | 157 | 2.00E−10 | 37 | (AF153422) lacZ [Cloning vector pTG8] |
| 1700 | LIB3602-093-Q6-K6-B7 | 416222 | BLASTX | 585 | 2.00E−60 | 98 | (D16504) alpha-tubulin [Chlorella vulgaris] |
| 1701 | LIB3602-104-Q1-K1-B7 | 416222 | BLASTX | 554 | 9.00E−57 | 99 | (D16504) alpha-tubulin [Chlorella vulgaris] |
| 1702 | LIB3602-117-Q1-K1-B7 | 416222 | BLASTX | 263 | 1.00E−43 | 100 | (D16504) alpha-tubulin [Chlorella vulgaris] |
| 1703 | LIB3602-002-P1-K6-E5 | 6056208 | BLASTX | 440 | 2.00E−43 | 50 | (AC009400) putative ATPase [Arabidopsis |
| | | | | | | | thaliana] |
| 1704 | LIB3602-065-Q1-K6-D5 | 6056208 | BLASTX | 191 | 2.00E−14 | 50 | (AC009400) putative ATPase [Arabidopsis |
| | | | | | | | thaliana] |
| 1705 | LIB3602-111-Q1-K1-A1 | 6056208 | BLASTX | 187 | 5.00E−14 | 50 | (AC009400) putative ATPase [Arabidopsis |
| | | | | | | | thaliana] |
| 1706 | LIB3602-029-Q6-K1-C10 | 1351122 | BLASTX | 502 | 1.00E−50 | 68 | THIAZOLE BIOSYNTHETIC ENZYME |
| | | | | | | | PRECURSOR (STRESS-INDUCIBLE PROTEIN |
| | | | | | | | STI35) >gi\|280494\|pir\|\|B37767 stress-inducible |
| | | | | | | | protein sti35 - fungus (Fusarium oxysporum) |
| | | | | | | | >gi\|168164\|gb\|AAA33341.1\| (M33643) STI35 |
| | | | | | | | protein [Fusarium oxysporum] |
| | | | | | | | >gi\|6045153\|dbj\|BAA85305.1\| ( |
| 1707 | LIB3602-088-Q6-K6-G1 | 1351122 | BLASTX | 477 | 1.00E−47 | 67 | THIAZOLE BIOSYNTHETIC ENZYME |
| | | | | | | | PRECURSOR (STRESS-INDUCIBLE PROTEIN |
| | | | | | | | STI35) >gi\|280494\|pir\|\|B37767 stress-inducible |
| | | | | | | | protein sti35 - fungus (Fusarium oxysporum) |
| | | | | | | | >gi\|168164\|gb\|AAA33341.1\| (M33643) STI35 |
| | | | | | | | protein [Fusarium oxysporum] |
| | | | | | | | >gi\|6045153\|dbj\|BAA85305.1\| ( |
| 1708 | LIB3602-103-Q1-K1-G10 | 1351122 | BLASTX | 391 | 1.00E−37 | 66 | THIAZOLE BIOSYNTHETIC ENZYME |
| | | | | | | | PRECURSOR (STRESS-INDUCIBLE PROTEIN |
| | | | | | | | STI35) >gi\|280494\|pir\|\|B37767 stress-inducible |
| | | | | | | | protein sti35 - fungus (Fusarium oxysporum) |
| | | | | | | | >gi\|168164\|gb\|AAA33341.1\| (M33643) STI35 |
| | | | | | | | protein [Fusarium oxysporum] |
| | | | | | | | >gi\|6045153\|dbj\|BAA85305.1\| ( |
| 1709 | LIB3602-062-Q6-K1-A9 | 3806118 | BLASTX | 182 | 1.00E−13 | 79 | (AF097201) 40S ribosomal protein S8 |
| | | | | | | | [Schizophyllum commune] |
| 1710 | LIB3602-062-Q6-K1-A10 | 3806118 | BLASTX | 181 | 1.00E−13 | 75 | (AF097201) 40S ribosomal protein S8 |
| | | | | | | | [Schizophyllum commune] |
| 1711 | LIB3602-007-Q1-K1-E11 | 3806118 | BLASTX | 165 | 9.00E−12 | 84 | (AF097201) 40S ribosomal protein S8 |
| | | | | | | | [Schizophyllum commune] |
| 1712 | LIB3602-113-Q1-K1-G6 | 132099 | BLASTX | 189 | 3.00E−14 | 92 | RIBULOSE BISPHOSPHATE CARBOXYLASE |
| | | | | | | | SMALL CHAIN PW9 PRECURSOR (RUBISCO |
| | | | | | | | SMALL SUBUNIT PW9) >gi\|68091\|pir\|\|RKWTS9 |
| | | | | | | | ribulose-bisphosphate carboxylase (EC 4.1.1.39) |
| | | | | | | | small chain precursor (clone pW9) - wheat |
| | | | | | | | >gi\|170771\|gb\|AAA34302.1\| (M37477) ribulose- |
| | | | | | | | 1,5-bisphos |
| 1713 | LIB3602-092-Q6-K6-G6 | 132099 | BLASTX | 185 | 8.00E−14 | 97 | RIBULOSE BISPHOSPHATE CARBOXYLASE |
| | | | | | | | SMALL CHAIN PW9 PRECURSOR (RUBISCO |
| | | | | | | | SMALL SUBUNIT PW9) >gi\|68091\|pir\|\|RKWTS9 |
| | | | | | | | ribulose-bisphosphate carboxylase (EC 4.1.1.39) |
| | | | | | | | small chain precursor (clone pW9) - wheat |
| | | | | | | | >gi\|170771\|gb\|AAA34302.1\| (M37477) ribulose- |
| | | | | | | | 1,5-bisphos |
| 1714 | LIB3602-120-Q1-K1-G6 | 132099 | BLASTX | 172 | 2.00E−12 | 100 | RIBULOSE BISPHOSPHATE CARBOXYLASE |
| | | | | | | | SMALL CHAIN PW9 PRECURSOR (RUBISCO |
| | | | | | | | SMALL SUBUNIT PW9) >gi\|68091\|pir\|\|RKWTS9 |
| | | | | | | | ribulose-bisphosphate carboxylase (EC 4.1.1.39) |
| | | | | | | | small chain precursor (clone pW9) - wheat |
| | | | | | | | >gi\|170771\|gb\|AAA34302.1\| (M37477) ribulose- |
| | | | | | | | 1,5-bisphos |
| 1715 | LIB3602-109-Q1-K1-F3 | 6554488 | BLASTX | 343 | 4.00E−32 | 62 | (AC012394) putative small nuclear |
| | | | | | | | ribonucleoprotein Sm D3 [Arabidopsis thaliana] |
| | | | | | | | >gi\|6573711\|gb\|AAF17631.1\|AC009978_7 |
| | | | | | | | (AC009978) T23E18.23 [Arabidopsis thaliana] |
| 1716 | LIB3602-074-Q1-K1-G10 | 6554488 | BLASTX | 327 | 4.00E−30 | 61 | (AC012394) putative small nuclear |
| | | | | | | | ribonucleoprotein Sm D3 [Arabidopsis thaliana] |
| | | | | | | | >gi\|6573711\|gb\|AAF17631.1\|AC009978_7 |
| | | | | | | | (AC009978) T23E18.23 [Arabidopsis thaliana] |
| 1717 | LIB3602-107-Q1-K1-A9 | 6554488 | BLASTX | 319 | 3.00E−29 | 62 | (AC012394) putative small nuclear |
| | | | | | | | ribonucleoprotein Sm D3 [Arabidopsis thaliana] |
| | | | | | | | >gi\|6573711\|gb\|AAF17631.1\|AC009978_7 |
| | | | | | | | (AC009978) T23E18.23 [Arabidopsis thaliana] |
| 1718 | LIB3602-074-Q1-K1-H11 | 417103 | BLASTX | 627 | 3.00E−65 | 99 | HISTONE H3.2, MINOR >gi\|282871\|pir\|\|S24346 |
| | | | | | | | histone H3.3-like protein - Arabidopsis thaliana |
| | | | | | | | >gi\|16324\|emb\|CAA42957.1\| (X60429) histone |
| | | | | | | | H3.3 like protein [Arabidopsis thaliana] |
| | | | | | | | >gi\|404825\|emb\|CAA42958.1\| (X60429) histone |
| | | | | | | | H3.3 like protein [Arabidopsis thal |
| 1719 | LIB3602-087-Q6-K1-E12 | 417103 | BLASTX | 425 | 1.00E−58 | 98 | HISTONE H3.2, MINOR >gi\|282871\|pir\|\|S24346 |
| | | | | | | | histone H3.3-like protein - Arabidopsis thaliana |
| | | | | | | | >gi\|16324\|emb\|CAA42957.1\| (X60429) histone |
| | | | | | | | H3.3 like protein [Arabidopsis thaliana] |
| | | | | | | | >gi\|404825\|emb\|CAA42958.1\| (X60429) histone |
| | | | | | | | H3.3 like protein [Arabidopsis thal |
| 1720 | LIB3602-007-Q1-K1-C5 | 417103 | BLASTX | 539 | 6.00E−55 | 94 | HISTONE H3.2, MINOR >gi\|282871\|pir\|\|S24346 |
| | | | | | | | histone H3.3-like protein - Arabidopsis thaliana |
| | | | | | | | >gi\|16324\|emb\|CAA42957.1\| (X60429) histone |
| | | | | | | | H3.3 like protein [Arabidopsis thaliana] |
| | | | | | | | >gi\|404825\|emb\|CAA42958.1\| (X60429) histone |
| | | | | | | | H3.3 like protein [Arabidopsis thal |
| 1721 | LIB3602-073-Q1-K1-G3 | 4512679 | BLASTX | 595 | 2.00E−61 | 80 | (AC006931) 60S ribosomal protein L11B |
| | | | | | | | [Arabidopsis thaliana] |
| 1722 | LIB3602-017-Q6-K1-E1 | 4512679 | BLASTX | 484 | 1.00E−48 | 84 | (AC006931) 60S ribosomal protein L11B |
| | | | | | | | [Arabidopsis thaliana] |
| 1723 | LIB3602-009-Q6-K1-H9 | 4512679 | BLASTX | 309 | 3.00E−28 | 82 | (AC006931) 60S ribosomal protein L11B |
| | | | | | | | [Arabidopsis thaliana] |
| 1724 | LIB3602-040-Q6-K1-H4 | 7488407 | BLASTX | 308 | 4.00E−28 | 55 | ubiquinol-cytochrome-c reductase (EC 1.10.2.2) |
| | | | | | | | 14K chain - Arabidopsis thaliana |
| | | | | | | | >gi\|4049349\|emb\|CAA22574.1\| (AL034567) |
| | | | | | | | ubiquinol-cytochrome c reductase-like protein |
| | | | | | | | [Arabidopsis thaliana] |
| | | | | | | | >gi\|7270151\|emb\|CAB79964.1\| (AL161581) |
| | | | | | | | ubiquinol-cytochrome c redu |
| 1725 | LIB3602-108-Q1-K1-H11 | 7488407 | BLASTX | 277 | 2.00E−24 | 53 | ubiquinol-cytochrome-c reductase (EC 1.10.2.2) |
| | | | | | | | 14K chain - Arabidopsis thaliana |
| | | | | | | | >gi\|4049349\|emb\|CAA22574.1\| (AL034567) |
| | | | | | | | ubiquinol-cytochrome c reductase-like protein |
| | | | | | | | [Arabidopsis thaliana] |
| | | | | | | | >gi\|7270151\|emb\|CAB79964.1\| (AL161581) |
| | | | | | | | ubiquinol-cytochrome c redu |
| 1726 | LIB3602-046-Q6-K1-D4 | 7488407 | BLASTX | 236 | 1.00E−19 | 63 | ubiquinol- cytochrome-c reductase (EC 1.10.2.2) |
| | | | | | | | 14K chain - Arabidopsis thaliana |
| | | | | | | | >gi\|4049349\|emb\|CAA22574.1\| (AL034567) |
| | | | | | | | ubiquinol-cytochrome c reductase-like protein |
| | | | | | | | [Arabidopsis thaliana] |
| | | | | | | | >gi\|7270151\|emb\|CAB79964.1\| (AL161581) |
| | | | | | | | ubiquinol-cytochrome c redu |
| 1727 | LIB3602-040-Q6-K1-F1 | 131183 | BLASTX | 315 | 8.00E−29 | 63 | PHOTOSYSTEM I REACTION CENTRE |
| | | | | | | | SUBUNIT III PRECURSOR (LIGHT- |
| | | | | | | | HARVESTING COMPLEX I 17 KD PROTEIN) |
| | | | | | | | (PSI-F) (P21 PROTEIN) >gi\|81244\|pir\|\|S04133 |
| | | | | | | | photosystem I chain III precursor - Chlamydomonas |
| | | | | | | | reinhardtii >gi\|18184\|emb\|CAA31849.1\| (X13495) |
| | | | | | | | P21 precursor prote |
| 1728 | LIB3602-045-Q6-K1-B8 | 131183 | BLASTX | 290 | 7.00E−26 | 61 | PHOTOSYSTEM I REACTION CENTRE |
| | | | | | | | SUBUNIT III PRECURSOR (LIGHT- |
| | | | | | | | HARVESTING COMPLEX I 17 KD PROTEIN) |
| | | | | | | | (PSI-F) (P21 PROTEIN) >gi\|81244\|pir\|\|S04133 |
| | | | | | | | photosystem I chain III precursor - Chlamydomonas |
| | | | | | | | reinhardtii >gi\|18184\|emb\|CAA31849.1\| (X13495) |
| | | | | | | | P21 precursor prote |
| 1729 | LIB3602-114-Q1-K1-C1 | 131183 | BLASTX | 288 | 1.00E−25 | 62 | PHOTOSYSTEM I REACTION CENTRE |
| | | | | | | | SUBUNIT III PRECURSOR (LIGHT- |
| | | | | | | | HARVESTING COMPLEX I 17 KD PROTEIN) |
| | | | | | | | (PSI-F) (P21 PROTEIN) >gi\|81244\|pir\|\|S04133 |
| | | | | | | | photosystem I chain III precursor - Chlamydomonas |
| | | | | | | | reinhardtii >gi\|18184\|emb\|CAA31849.1\| (X13495) |
| | | | | | | | P21 precursor prote |
| 1730 | LIB3602-019-Q6-K1-H12 | 3914658 | BLASTX | 331 | 1.00E−30 | 47 | 50S RIBOSOMAL PROTEIN L24, |
| | | | | | | | CHLOROPLAST PRECURSOR |
| | | | | | | | >gi\|1694974\|emb\|CAA70851.1\| (Y09635) plastid |
| | | | | | | | ribosomal protein [Arabidopsis thaliana] |
| 1731 | LIB3602-025-Q6-K1-F11 | 3914658 | BLASTX | 331 | 1.00E−30 | 47 | 50S RIBOSOMAL PROTEIN L24, |
| | | | | | | | CHLOROPLAST PRECURSOR |
| | | | | | | | >gi\|1694974\|emb\|CAA70851.1\| (Y09635) plastid |
| | | | | | | | ribosomal protein [Arabidopsis thaliana] |
| 1732 | LIB3602-083-Q6-K6-B7 | 3914658 | BLASTX | 331 | 2.00E−30 | 47 | 50S RIBOSOMAL PROTEIN L24, |
| | | | | | | | CHLOROPLAST PRECURSOR |
| | | | | | | | >gi\|1694974\|emb\|CAA70851.1\| (Y09635) plastid |
| | | | | | | | ribosomal protein [Arabidopsis thaliana] |
| 1733 | LIB3602-073-Q1-K1-E6 | 7428008 | BLASTX | 565 | 5.00E−58 | 54 | calcium-dependent protein kinase (EC 2.7.1.—), |
| | | | | | | | calmodulin-independent - maize (fragment) |
| | | | | | | | >gi\|639722\|gb\|AAA61682.1\| (L27484) calcium- |
| | | | | | | | dependent protein kinase [Zea mays] |
| 1734 | LIB3602-093-Q6-K6-F10 | 7428008 | BLASTX | 288 | 2.00E−25 | 45 | calcium-dependent protein kinase (EC 2.7.1.—), |
| | | | | | | | calmodulin-independent - maize (fragment) |
| | | | | | | | >gi\|639722\|gb\|AAA61682.1\| (L27484) calcium- |
| | | | | | | | dependent protein kinase [Zea mays] |
| 1735 | LIB3602-119-Q1-K1-F10 | 7428008 | BLASTX | 273 | 5.00E−24 | 45 | calcium-dependent protein kinase (EC 2.7.1.—), |
| | | | | | | | calmodulin-independent - maize (fragment) |
| | | | | | | | >gi\|639722\|gb\|AAA61682.1\| (L27484) calcium- |
| | | | | | | | dependent protein kinase [Zea mays] |
| 1736 | LIB3602-040-Q6-K1-A5 | 5902365 | BLASTX | 662 | 1.00E−69 | 86 | (AC009322) Putative splicing factor Prp8 |
| | | | | | | | [Arabidopsis thaliana] |
| 1737 | LIB3602-054-Q6-K1-F12 | 5902365 | BLASTX | 177 | 1.00E−12 | 50 | (AC009322) Putative splicing factor Prp8 |
| | | | | | | | [Arabidopsis thaliana] |
| 1738 | LIB3602-056-Q6-K1-D12 | 2492861 | BLASTX | 561 | 1.00E−57 | 77 | GLUTAMATE-1-SEMIALDEHYDE 2,1- |
| | | | | | | | AMINOMUTASE PRECURSOR (GSA) |
| | | | | | | | (GLUTAMATE-1-SEMIALDEHYDE |
| | | | | | | | AMINOTRANSFERASE) (GSA-AT) |
| | | | | | | | >gi\|629466\|pir\|\|S43787 glutamate-1-semialdehyde |
| | | | | | | | 2,1-aminomutase (EC 5.4.3.8) - Chlamydomonas |
| | | | | | | | reinhardtii >gi\|424151\|gb\|AAA18861.1\| (U03632) |
| | | | | | | | gluta |
| 1739 | LIB3602-044-Q6-K1-G6 | 2492861 | BLASTX | 555 | 6.00E−57 | 76 | GLUTAMATE-1-SEMIALDEHYDE 2,1- |
| | | | | | | | AMINOMUTASE PRECURSOR (GSA) |
| | | | | | | | (GLUTAMATE-1-SEMIALDEHYDE |
| | | | | | | | AMINOTRANSFERASE) (GSA-AT) |
| | | | | | | | >gi\|629466\|pir\|\|S43787 glutamate-1-semialdehyde |
| | | | | | | | 2,1-aminomutase (EC 5.4.3.8) - Chlamydomonas |
| | | | | | | | reinhardtii >gi\|424151\|gb\|AAA18861.1\| (U03632) |
| | | | | | | | gluta |
| 1740 | LIB3602-035-Q1-K1-B11 | 6647650 | BLASTX | 504 | 8.00E−54 | 62 | NADH-UBIQUINONE OXIDOREDUCTASE 24 KD |
| | | | | | | | SUBUNIT PRECURSOR |
| | | | | | | | >gi\|7427765\|pir\|\|T01091 NADH dehydrogenase |
| | | | | | | | (ubiquinone) (EC 1.6.5.3) 24K chain homolog - |
| | | | | | | | Arabidopsis thaliana |
| | | | | | | | >gi\|3892051\|gb\|AAC78260.1\|AAC78260 |
| | | | | | | | (AC002330) predicted NADH dehydrogenase 24 kD |
| | | | | | | | subunit [ |
| 1741 | LIB3602-034-Q6-K1-B11 | 6647650 | BLASTX | 490 | 3.00E−49 | 61 | NADH-UBIQUINONE OXIDOREDUCTASE 24 KD |
| | | | | | | | SUBUNIT PRECURSOR |
| | | | | | | | >gi\|7427765\|pir\|\|T01091 NADH dehydrogenase |
| | | | | | | | (ubiquinone) (EC 1.6.5.3) 24K chain homolog - |
| | | | | | | | Arabidopsis thaliana |
| | | | | | | | >gi\|3892051\|gb\|AAC78260.1\|AAC78260 |
| | | | | | | | (AC002330) predicted NADH dehydrogenase 24 kD |
| | | | | | | | subunit [ |
| 1742 | LIB3602-106-Q1-K1-B10 | 7438140 | BLASTX | 521 | 8.00E−53 | 67 | acetyl-CoA carboxylase (EC 6.4.1.2), biotin |
| | | | | | | | carboxylase chain precursor - soybean |
| | | | | | | | >gi\|3219361\|gb\|AAC23573.1\| (AF068249) biotin |
| | | | | | | | carboxylase precursor [Glycine max] |
| 1743 | LIB3602-100-Q1-K1-B10 | 7438140 | BLASTX | 485 | 1.00E−48 | 65 | acetyl-CoA carboxylase (EC 6.4.1.2), biotin |
| | | | | | | | carboxylase chain precursor - soybean |
| | | | | | | | >gi\|3219361\|gb\|AAC23573.1\| (AF068249) biotin |
| | | | | | | | carboxylase precursor [Glycine max] |
| 1744 | LIB3602-085-Q6-K1-F9 | 115575 | BLASTX | 447 | 4.00E−44 | 51 | PHOSPHOENOLPYRUVATE CARBOXYLASE 1 |
| | | | | | | | (PEPCASE) >gi\|68032\|pir\|\|QYIX1 |
| | | | | | | | phosphoenolpyruvate carboxylase (EC 4.1.1.31) 1 - |
| | | | | | | | common ice plant >gi\|19536\|emb\|CAA31956.1\| |
| | | | | | | | (X13660) phosphoenolpyruvate carboxylase |
| | | | | | | | (AA 1-966) |
| | | | | | | | [Mesembryanthemum crystallinum] |
| | | | | | | | >gi\|22559\|e |
| 1745 | LIB3602-022-Q6-K1-A2 | 115575 | BLASTX | 338 | 1.00E−31 | 50 | PHOSPHOENOLPYRUVATE CARBOXYLASE 1 |
| | | | | | | | (PEPCASE) >gi\|68032\|pir\|\|QYIX1 |
| | | | | | | | phosphoenolpyruvate carboxylase (EC 4.1.1.31) 1 - |
| | | | | | | | common ice plant >gi\|19536\|emb\|CAA31956.1\| |
| | | | | | | | (X13660) phosphoenolpyruvate carboxylase |
| | | | | | | | (AA 1-966) |
| | | | | | | | [Mesembryanthemum crystallinum] |
| | | | | | | | >gi\|22559\|e |
| 1746 | LIB3602-078-Q6-K6-C7 | 7437844 | BLASTX | 382 | 2.00E−36 | 46 | probable long-chain-fatty-acid--CoA ligase (EC |
| | | | | | | | 6.2.1.3) isoform 1 - rape |
| | | | | | | | >gi\|1617268\|emb\|CAA96523.1\| (Z72153) acyl CoA |
| | | | | | | | synthetase [Brassica napus] |
| 1747 | LIB3602-038-Q6-K1-B7 | 7437844 | BLASTX | 301 | 4.00E−27 | 47 | probable long-chain-fatty-acid--CoA ligase (EC |
| | | | | | | | 6.2.1.3) isoform 1 - rape |
| | | | | | | | >gi\|1617268\|emb\|CAA96523.1\| (Z72153) acyl CoA |
| | | | | | | | synthetase [Brassica napus] |
| 1748 | LIB3602-040-Q6-K1-G4 | 166985 | BLASTX | 532 | 3.00E−54 | 60 | (J04202) alpha-amylase [Hordeum vulgare] |
| 1749 | LIB3602-056-Q6-K1-E11 | 166985 | BLASTX | 500 | 1.00E−50 | 64 | (J04202) alpha-amylase [Hordeum vulgare] |
| 1750 | LIB3602-085-Q6-K1-D6 | 5708095 | BLASTX | 524 | 3.00E−53 | 59 | (AJ245502) ATP synthase gamma chain, |
| | | | | | | | chloroplast precursor [Arabidopsis thaliana] |
| 1751 | LIB3602-063-Q1-K6-E7 | 5708095 | BLASTX | 493 | 1.00E−49 | 55 | (AJ245502) ATP synthase gamma chain, |
| | | | | | | | chloroplast precursor [Arabidopsis thaliana] |
| 1752 | LIB3602-014-Q6-K1-H2 | 7481092 | BLASTX | 273 | 7.00E−24 | 52 | probable oxidoreductase - Streptomyces coelicolor |
| | | | | | | | >gi\|2815335\|emb\|CAA16459.1\| (AL021529) |
| | | | | | | | SC10A5.27c, possible oxidoreductase, len: 326 aa; |
| | | | | | | | so me similarity to many members of the SHORT- |
| | | | | | | | CHAIN DEHYDROGEN ASES/REDUCTASES |
| | | | | | | | FAMILY (SDR) eg. YGFF_ECOLI P52037 hy |
| 1753 | LIB3602-058-Q6-K1-G9 | 7481092 | BLASTX | 191 | 2.00E−14 | 40 | probable oxidoreductase - Streptomyces coelicolor |
| | | | | | | | >gi\|2815335\|emb\|CAA16459.1\| (AL021529) |
| | | | | | | | SC10A5.27c, possible oxidoreductase, len: 326 aa; |
| | | | | | | | so me similarity to many members of the SHORT- |
| | | | | | | | CHAIN DEHYDROGEN ASES/REDUCTASES |
| | | | | | | | FAMILY (SDR) eg. YGFF_ECOLI P52037 hy |
| 1754 | LIB3602-073-Q1-K1-A3 | 5902400 | BLASTX | 419 | 7.00E−41 | 51 | (AC008148) Unknown protein [Arabidopsis |
| | | | | | | | thaliana] |
| 1755 | LIB3602-010-Q6-K1-G11 | 5902400 | BLASTX | 410 | 7.00E−40 | 50 | (AC008148) Unknown protein [Arabidopsis |
| | | | | | | | thaliana] |
| 1756 | LIB3602-019-Q6-K1-G6 | 6403490 | BLASTX | 375 | 9.00E−36 | 51 | (AC010871) putative SCO1 protein [Arabidopsis |
| | | | | | | | thaliana] |
| 1757 | LIB3602-057-Q6-K1-E4 | 6403490 | BLASTX | 321 | 2.00E−29 | 60 | (AC010871) putative SCO1 protein [Arabidopsis |
| | | | | | | | thaliana] |
| 1758 | LIB3602-041-Q6-K1-D10 | 322391 | BLASTX | 346 | 2.00E−32 | 51 | chlorophyll a/b-binding protein Lhcb5 - Scotch pine |
| | | | | | | | >gi\|22750\|emb\|CAA78900.1\| (Z16408) Lhcb5 |
| | | | | | | | protein [Pinus sylvestris] |
| | | | | | | | >gi\|1093837\|prf\|\|2104448A Lhcb5 gene [Pinus |
| | | | | | | | sylvestris] |
| 1759 | LIB3602-072-Q1-K1-D5 | 322391 | BLASTX | 346 | 3.00E−32 | 51 | chlorophyll a/b-binding protein Lhcb5 - Scotch pine |
| | | | | | | | >gi\|22750\|emb\|CAA78900.1\| (Z16408) Lhcb5 |
| | | | | | | | protein [Pinus sylvestris] |
| | | | | | | | >gi\|1093837\|prf\|\|2104448A Lhcb5 gene [Pinus |
| | | | | | | | sylvestris] |
| 1760 | LIB3602-047-Q6-K1-F10 | 2494745 | BLASTX | 385 | 5.00E−37 | 81 | GLUTAMINE SYNTHETASE, CHLOROPLAST |
| | | | | | | | PRECURSOR (GLUTAMATE-AMMONIA |
| | | | | | | | LIGASE) (GS2) >gi\|7437923\|pir\|\|T08090 probable |
| | | | | | | | glutamate-ammonia ligase (EC 6.3.1.2), chloroplast - |
| | | | | | | | Chlamydomonas reinhardtii |
| | | | | | | | >gi\|1353878\|gb\|AAB01818.1\| (U46208) glutamine |
| | | | | | | | synthetase [Chlamy |
| 1761 | LIB3602-090-Q6-K6-D5 | 2494745 | BLASTX | 256 | 2.00E−22 | 78 | GLUTAMINE SYNTHETASE, CHLOROPLAST |
| | | | | | | | PRECURSOR (GLUTAMATE-AMMONIA |
| | | | | | | | LIGASE) (GS2) >gi\|7437923\|pir\|\|T08090 probable |
| | | | | | | | glutamate-ammonia ligase (EC 6.3.1.2), chloroplast - |
| | | | | | | | Chlamydomonas reinhardtii |
| | | | | | | | >gi\|1353878\|gb\|AAB01818.1\| (U46208) glutamine |
| | | | | | | | synthetase [Chlamy |
| 1762 | LIB3602-062-Q6-K1-B4 | 6671953 | BLASTX | 401 | 8.00E−39 | 62 | (AC016795) putative pre-rRNA processing protein, |
| | | | | | | | 5′ partial [Arabidopsis thaliana] |
| 1763 | LIB3602-062-Q6-K1-B5 | 6671953 | BLASTX | 245 | 1.00E−20 | 53 | (AC016795) putative pre-rRNA processing protein, |
| | | | | | | | 5′ partial [Arabidopsis thaliana] |
| 1764 | LIB3602-074-Q1-K1-E4 | 1085669 | BLASTX | 197 | 6.00E−15 | 38 | methylcrotonoyl-CoA carboxylase (EC 6.4.1.4) |
| | | | | | | | biotin-binding chain - tomato (fragment) |
| | | | | | | | >gi\|497181\|gb\|AAA19157.1\| (U07745) biotin- |
| | | | | | | | containing subunit of methylcrotonyl-CoA |
| | | | | | | | carboxylase [Lycopersicon esculentum] |
| 1765 | LIB3602-087-Q6-K1-H6 | 1085669 | BLASTX | 197 | 7.00E−15 | 38 | methylcrotonoyl-CoA carboxylase (EC 6.4.1.4) |
| | | | | | | | biotin-binding chain - tomato (fragment) |
| | | | | | | | >gi\|497181\|gb\|AAA19157.1\| (U07745) biotin- |
| | | | | | | | containing subunit of methylcrotonyl-CoA |
| | | | | | | | carboxylase [Lycopersicon esculentum] |
| 1766 | LIB3602-077-Q6-K6-H8 | 1730560 | BLASTX | 226 | 3.00E−18 | 63 | ALPHA-GLUCAN PHOSPHORYLASE, H |
| | | | | | | | ISOZYME (STARCH PHOSPHORYLASE H) |
| | | | | | | | >gi\|7433824\|pir\|\|T12091 starch phosphorylase (EC |
| | | | | | | | 2.4.1.1) H, cytosolic isoform - fava bean |
| | | | | | | | >gi\|510932\|emb\|CAA84494.1\| (Z35117) alpha 1,4- |
| | | | | | | | glucan phosphorylase type H [Vicia faba] |
| 1767 | LIB3602-088-Q6-K6-D10 | 1730560 | BLASTX | 226 | 3.00E−18 | 63 | ALPHA-GLUCAN PHOSPHORYLASE, H |
| | | | | | | | ISOZYME (STARCH PHOSPHORYLASE H) |
| | | | | | | | >gi\|7433824\|pir\|\|T12091 starch phosphorylase (EC |
| | | | | | | | 2.4.1.1) H, cytosolic isoform - fava bean |
| | | | | | | | >gi\|510932\|emb\|CAA84494.1\| (Z35117) alpha 1,4- |
| | | | | | | | glucan phosphorylase type H [Vicia faba] |
| 1768 | LIB3602-016-Q6-K1-D10 | 7487536 | BLASTX | 332 | 8.00E−31 | 48 | hypothetical protein T29H11.60 - Arabidopsis |
| | | | | | | | thaliana >gi\|4678346\|emb\|CAB41156.1\| |
| | | | | | | | (AL049659) putative protein [Arabidopsis thaliana] |
| 1769 | LIB3602-017-Q6-K1-B4 | 7487536 | BLASTX | 182 | 3.00E−13 | 72 | hypothetical protein T29H11.60 - Arabidopsis |
| | | | | | | | thaliana >gi\|4678346\|emb\|CAB41156.1\| |
| | | | | | | | (AL049659) putative protein [Arabidopsis thaliana] |
| 1770 | LIB3602-025-Q6-K1-A12 | 6468487 | BLASTX | 352 | 4.00E−33 | 68 | (AJ251083) putative cytosolic malate |
| | | | | | | | dehydrogenase [Beta vulgaris] |
| 1771 | LIB3602-037-Q6-K1-C12 | 6468487 | BLASTX | 235 | 2.00E−19 | 71 | (AJ251083) putative cytosolic malate |
| | | | | | | | dehydrogenase [Beta vulgaris] |
| 1772 | LIB3602-050-Q6-K1-B10 | 167408 | BLASTN | 38 | 1.00E−11 | 91 | C. reinhardtii encoding chlorophyll a/b-binding |
| | | | | | | | protein (cabII-1) gene, complete cds |
| 1773 | LIB3602-019-Q6-K1-A6 | 167408 | BLASTN | 33 | 9.00E−09 | 89 | C. reinhardtii encoding chlorophyll a/b-binding |
| | | | | | | | protein (cabII-1) gene, complete cds |
| 1774 | LIB3602-011-Q6-K1-D11 | 4115942 | BLASTX | 212 | 1.00E−16 | 50 | (AF118223) contains similarity to eukaryotic |
| | | | | | | | protein kinase domains (Pfam: PF00069, |
| | | | | | | | score = 238.4, E = 1e−67, N = 1) and EF |
| | | | | | | | hand domains |
| | | | | | | | (Pfam: PF00036, score = 109.0, E = 8.9e−29, N = 5) |
| | | | | | | | [Arabidopsis thaliana] |
| | | | | | | | >gi\|7267229\|emb\|CAB80836.1\| (AL161501) |
| | | | | | | | putative calcium |
| 1775 | LIB3602-053-Q6-K1-B11 | 4115942 | BLASTX | 153 | 1.00E−09 | 49 | (AF118223) contains similarity to eukaryotic |
| | | | | | | | protein kinase domains (Pfam: PF00069, |
| | | | | | | | score = 238.4, E = 1e−67, N = 1) and EF |
| | | | | | | | hand domains |
| | | | | | | | (Pfam: PF00036, score = 109.0, E = 8.9e−29, N = 5) |
| | | | | | | | [Arabidopsis thaliana] |
| | | | | | | | >gi\|7267229\|emb\|CAB80836.1\| (AL161501) |
| | | | | | | | putative calcium |
| 1776 | LIB3602-064-Q1-K6-F10 | 7485715 | BLASTX | 288 | 1.00E−25 | 59 | hypothetical protein F18E5.180 - Arabidopsis |
| | | | | | | | thaliana >gi\|3080400\|emb\|CAA18720.1\| |
| | | | | | | | (AL022603) putative protein [Arabidopsis thaliana] |
| | | | | | | | >gi\|4455264\|emb\|CAB36800.1\| (AL035527) |
| | | | | | | | putative protein [Arabidopsis thaliana] |
| | | | | | | | >gi\|7268953\|emb\|CAB81263.1\| (AL161555) puta |
| 1777 | LIB3602-059-Q6-K1-B7 | 7485715 | BLASTX | 173 | 2.00E−12 | 50 | hypothetical protein F18E5.180 - Arabidopsis |
| | | | | | | | thaliana >gi\|3080400\|emb\|CAA18720.1\| |
| | | | | | | | (AL022603) putative protein [Arabidopsis thaliana] |
| | | | | | | | >gi\|4455264\|emb\|CAB36800.1\| (AL035527) |
| | | | | | | | putative protein [Arabidopsis thaliana] |
| | | | | | | | >gi\|7268953\|emb\|CAB81263.1\| (AL161555) puta |
| 1778 | LIB3602-053-Q6-K1-G7 | 4514360 | BLASTX | 234 | 2.00E−19 | 63 | (AB013377) BsaA [Bacillus halodurans] |
| 1779 | LIB3602-055-Q6-K1-D11 | 4514360 | BLASTX | 216 | 3.00E−17 | 68 | (AB013377) BsaA [Bacillus halodurans] |
| 1780 | LIB3602-109-Q1-K1-H8 | 2232057 | BLASTX | 230 | 9.00E−19 | 59 | (AF000177) CaSm [Homo sapiens] |
| | | | | | | | >gi\|5262854\|emb\|CAB45865.1\| (AJ238094) Lsm1 |
| | | | | | | | protein [Homo sapiens] |
| 1781 | LIB3602-083-Q6-K6-H8 | 2232057 | BLASTX | 230 | 1.00E−18 | 59 | (AF000177) CaSm [Homo sapiens] |
| | | | | | | | >gi\|5262854\|emb\|CAB45865.1\| (AJ238094) Lsm1 |
| | | | | | | | protein [Homo sapiens] |
| 1782 | LIB3602-025-Q6-K1-C4 | 2462781 | BLASTX | 617 | 4.00E−64 | 67 | (U73175) carbamoyl phosphate synthetase small |
| | | | | | | | subunit [Arabidopsis thaliana] |
| 1783 | LIB3602-002-P1-K6-H9 | 2462781 | BLASTX | 254 | 1.00E−21 | 62 | (U73175) carbamoyl phosphate synthetase small |
| | | | | | | | subunit [Arabidopsis thaliana] |
| 1784 | LIB3602-093-Q6-K6-C8 | 7381458 | BLASTX | 472 | 4.00E−47 | 56 | (AF210322) UMP synthase [Oryza sativa] |
| 1785 | LIB3602-104-Q1-K1-C8 | 7381458 | BLASTX | 437 | 5.00E−43 | 57 | (AF210322) UMP synthase [Oryza sativa] |
| 1786 | LIB3602-104-Q1-K1-G9 | 1419687 | BLASTX | 425 | 1.00E−41 | 50 | (X98825) 40-kDa V-ATPase subunit [Manduca |
| | | | | | | | sexta] |
| 1787 | LIB3602-093-Q6-K6-G9 | 1419687 | BLASTX | 424 | 2.00E−41 | 50 | (X98825) 40-kDa V-ATPase subunit [Manduca |
| | | | | | | | sexta] |
| 1788 | LIB3602-095-Q6-K6-H1 | 2117676 | BLASTX | 692 | 7.00E−73 | 65 | superoxide dismutase (EC 1.15.1.1) (Fe) precursor - |
| | | | | | | | Chlamydomonas reinhardtii |
| | | | | | | | >gi\|726293\|gb\|AAB04944.1\| (U22416) superoxide |
| | | | | | | | dismutase precursor [Chlamydomonas reinhardtii] |
| 1789 | LIB3602-077-Q6-K6-F8 | 2117676 | BLASTX | 421 | 1.00E−42 | 66 | superoxide dismutase (EC 1.15.1.1) (Fe) precursor - |
| | | | | | | | Chlamydomonas reinhardtii |
| | | | | | | | >gi\|726293\|gb\|AAB04944.1\| (U22416) superoxide |
| | | | | | | | dismutase precursor [Chlamydomonas reinhardtii] |
| 1790 | LIB3602-059-Q6-K1-B1 | 6466939 | BLASTX | 177 | 1.00E−12 | 49 | (AC009176) unknown protein, 5′ partial |
| | | | | | | | [Arabidopsis thaliana] |
| 1791 | LIB3602-059-Q6-K1-B2 | 6466939 | BLASTX | 175 | 3.00E−12 | 47 | (AC009176) unknown protein, 5′ partial |
| | | | | | | | [Arabidopsis thaliana] |
| 1792 | LIB3602-067-Q1-K1-A8 | 6911856 | BLASTX | 248 | 2.00E−30 | 88 | (AL138649) putative protein [Arabidopsis thaliana] |
| 1793 | LIB3602-084-Q6-K1-G11 | 6911856 | BLASTX | 193 | 8.00E−26 | 81 | (AL138649) putative protein [Arabidopsis thaliana] |
| 1794 | LIB3602-072-Q1-K1-B8 | 130264 | BLASTX | 231 | 2.00E−19 | 77 | PLASTOCYANIN PRECURSOR (PC6-2) |
| | | | | | | | >gi\|99419\|pir\|\|A36569 plastocyanin precursor - |
| | | | | | | | Chlamydomonas reinhardtii |
| | | | | | | | >gi\|167401\|gb\|AAA33078.1\| (J05524) |
| | | | | | | | apoplastocyanin (PC6-2) precursor |
| | | | | | | | [Chlamydomonas reinhardtii] |
| | | | | | | | >gi\|167430\|gb\|AAA33089.1\| (L07282) plastocyanin |
| | | | | | | | [Chlam |
| 1795 | LIB3602-040-Q6-K1-F8 | 130264 | BLASTX | 227 | 6.00E−19 | 78 | PLASTOCYANIN PRECURSOR (PC6-2) |
| | | | | | | | >gi\|99419\|pir\|\|A36569 plastocyanin precursor - |
| | | | | | | | Chlamydomonas reinhardtii |
| | | | | | | | >gi\|167401\|gb\|AAA33078.1\| (J05524) |
| | | | | | | | apoplastocyanin (PC6-2) precursor |
| | | | | | | | [Chlamydomonas reinhardtii] |
| | | | | | | | >gi\|167430\|gb\|AAA33089.1\| (L07282) plastocyanin |
| | | | | | | | [Chlam |
| 1796 | LIB3602-060-Q6-K1-H1 | 4530100 | BLASTX | 345 | 3.00E−32 | 43 | (AF063418) elongation factor 1-alpha [Enchytraeus |
| | | | | | | | sp. ‘Enc’] |
| 1797 | LIB3602-063-Q1-K6-F10 | 4530100 | BLASTX | 272 | 8.00E−24 | 42 | (AF063418) elongation factor 1-alpha [Enchytraeus |
| | | | | | | | sp. ‘Enc’] |
| 1798 | LIB3602-059-Q6-K1-G2 | 6907102 | BLASTX | 218 | 2.00E−17 | 66 | (AP001129) ESTs |
| | | | | | | | AU032852(S15362), AU070591(S5037) correspond |
| | | | | | | | to a region of the predicted gene.; Similar to |
| | | | | | | | Caenorhabditis elegans cosmid T10C6. (Z93388) |
| | | | | | | | [Oryza sativa] |
| 1799 | LIB3602-059-Q6-K1-G3 | 6907102 | BLASTX | 189 | 5.00E−14 | 45 | (AP001129) ESTs |
| | | | | | | | AU032852(S15362), AU070591(S5037) correspond |
| | | | | | | | to a region of the predicted gene.; Similar to |
| | | | | | | | Caenorhabditis elegans cosmid T10C6. (Z93388) |
| | | | | | | | [Oryza sativa] |
| 1800 | LIB3602-070-Q1-K1-D8 | 7340289 | BLASTX | 251 | 4.00E−21 | 44 | (AF245222) OP1 [Cucumis sativus] |
| 1801 | LIB3602-076-Q6-K6-B9 | 7340289 | BLASTX | 251 | 4.00E−21 | 44 | (AF245222) OP1 [Cucumis sativus] |
| 1802 | LIB3602-070-Q1-K1-A5 | 4972728 | BLASTX | 529 | 9.00E−54 | 57 | (AF132171) unknown [Drosophila melanogaster] |
| | | | | | | | >gi\|7300952\|gb\|AAF56091.1\| (AE003741) |
| | | | | | | | BcDNA: LD21504 gene product [Drosophila |
| | | | | | | | melanogaster] |
| 1803 | LIB3602-001-P1-K6-F12 | 4972728 | BLASTX | 382 | 1.00E−36 | 55 | (AF132171) unknown [Drosophila melanogaster] |
| | | | | | | | >gi\|7300952\|gb\|AAF56091.1\| (AE003741) |
| | | | | | | | BcDNA: LD21504 gene product [Drosophila |
| | | | | | | | melanogaster] |
| 1804 | LIB3602-045-Q6-K1-D11 | 914284 | BLASTX | 232 | 4.00E−19 | 84 | light-harvesting complex b type 3, Lhcb3 [Ginkgo |
| | | | | | | | biloba, 3-4 week old seedlings, Peptide Partial, 132 |
| | | | | | | | aa] |
| 1805 | LIB3602-058-Q6-K1-C10 | 914284 | BLASTX | 232 | 4.00E−19 | 84 | light-harvesting complex b type 3, Lhcb3 [Ginkgo |
| | | | | | | | biloba, 3-4 week old seedlings, Peptide Partial, 132 |
| | | | | | | | aa] |
| 1806 | LIB3602-076-Q6-K6-C11 | 4731372 | BLASTX | 191 | 4.00E−14 | 46 | (AF133838) papain-like cysteine protease |
| | | | | | | | [Sandersonia aurantiaca] |
| 1807 | LIB3602-112-Q1-K1-H2 | 4731372 | BLASTX | 175 | 3.00E−12 | 44 | (AF133838) papain-like cysteine protease |
| | | | | | | | [Sandersonia aurantiaca] |
| 1808 | LIB3602-050-Q6-K1-A12 | 6599226 | BLASTX | 346 | 2.00E−32 | 46 | (AL133613) hypothetical protein [Homo sapiens] |
| 1809 | LIB3602-050-Q6-K1-A11 | 6599226 | BLASTX | 305 | 1.00E−27 | 42 | (AL133613) hypothetical protein [Homo sapiens] |
| 1810 | LIB3602-115-Q1-K1-D7 | 7433571 | BLASTX | 636 | 2.00E−66 | 67 | probable |
| | | | | | | | phosphoribosylaminoimidazolecarboxamide |
| | | | | | | | formyltransferase (EC 2.1.2.3) - Arabidopsis |
| | | | | | | | thaliana >gi\|3033398\|gb\|AAC12842.1\| (AC004238) |
| | | | | | | | putative |
| | | | | | | | phosphoribosylaminoimidazolecarboxamide |
| | | | | | | | formyltransferase [Arabidopsis thaliana] |
| 1811 | LIB3602-115-Q1-K1-A7 | 7433571 | BLASTX | 550 | 2.00E−56 | 65 | probable |
| | | | | | | | phosphoribosylaminoimidazolecarboxamide |
| | | | | | | | formyltransferase (EC 2.1.2.3) - Arabidopsis |
| | | | | | | | thaliana >gi\|3033398\|gb\|AAC12842.1\| (AC004238) |
| | | | | | | | putative |
| | | | | | | | phosphoribosylaminoimidazolecarboxamide |
| | | | | | | | formyltransferase [Arabidopsis thaliana] |
| 1812 | LIB3602-005-Q1-K1-C11 | 3915430 | BLASTX | 280 | 1.00E−24 | 59 | HYPOTHETICAL 11.3 KD PROTEIN IN RIBE- |
| | | | | | | | PYKF INTERGENIC REGION PRECURSOR |
| | | | | | | | >gi\|7459798\|pir\|\|C64924 hypothetical protein b1667 - |
| | | | | | | | >gi\|1549278\|gb\|AAB47943.1\| |
| | | | | | | | (U68703) hypothetical protein [Escherichia coli] |
| | | | | | | | >gi\|1787956\|gb\|AAC74737.1\| (AE000262) o |
| 1813 | LIB3602-078-Q6-K6-C3 | 3915430 | BLASTX | 267 | 3.00E−23 | 60 | HYPOTHETICAL 11.3 KD PROTEIN IN RIBE- |
| | | | | | | | PYKF INTERGENIC REGION PRECURSOR |
| | | | | | | | >gi\|7459798\|pir\|\|C64924 hypothetical protein b1667 - |
| | | | | | | | Escherichia coli >gi\|1549278\|gb\|AAB47943.1\| |
| | | | | | | | (U68703) hypothetical protein [Escherichia coli] |
| | | | | | | | >gi\|1787956\|gb\|AAC74737.1\| (AE000262) o |
| 1814 | LIB3602-109-Q1-K1-B2 | 7484357 | BLASTX | 515 | 9.00E−63 | 83 | carbonate dehydratase (EC 4.2.1.1) 2 beta, |
| | | | | | | | mitochondrial - Chlamydomonas reinhardtii |
| | | | | | | | >gi\|1737488\|gb\|AAC49888.1\| (U80805) beta- |
| | | | | | | | carbonic anhydrase [Chlamydomonas reinhardtii] |
| 1815 | LIB3602-040-Q6-K1-F9 | 7484357 | BLASTX | 233 | 3.00E−19 | 74 | carbonate dehydratase (EC 4.2.1.1) 2 beta, |
| | | | | | | | mitochondrial - Chlamydomonas reinhardtii |
| | | | | | | | >gi\|1737488\|gb\|AAC49888.1\| (U80805) beta- |
| | | | | | | | carbonic anhydrase [Chlamydomonas reinhardtii] |
| 1816 | LIB3602-004-Q1-K1-D9 | 7298333 | BLASTX | 185 | 2.00E−13 | 32 | (AE003652) CG17905 gene product [Drosophila |
| | | | | | | | melanogaster] |
| 1817 | LIB3602-085-Q6-K1-E3 | 7298333 | BLASTX | 171 | 9.00E−12 | 29 | (AE003652) CG17905 gene product [Drosophila |
| | | | | | | | melanogaster] |
| 1818 | LIB3602-029-Q6-K1-A4 | 7435330 | BLASTX | 412 | 4.00E−40 | 48 | alpha-amylase (EC 3.2.1.1) - Arabidopsis thaliana |
| | | | | | | | >gi\|4455243\|emb\|CAB36742.1\| (AL035523) alpha- |
| | | | | | | | amylase-like protein [Arabidopsis thaliana] |
| | | | | | | | >gi\|7269350\|emb\|CAB79409.1\| (AL161562) alpha- |
| | | | | | | | amylase-like protein [Arabidopsis thaliana] |
| 1819 | LIB3602-051-Q6-K1-F9 | 7435330 | BLASTX | 401 | 7.00E−39 | 50 | alpha-amylase (EC 3.2.1.1) - Arabidopsis thaliana |
| | | | | | | | >gi\|4455243\|emb\|CAB36742.1\| (AL035523) alpha- |
| | | | | | | | amylase-like protein [Arabidopsis thaliana] |
| | | | | | | | >gi\|7269350\|emb\|CAB79409.1\| (AL161562) alpha- |
| | | | | | | | amylase-like protein [Arabidopsis thaliana] |
| 1820 | LIB3602-108-Q1-K1-F10 | 7443228 | BLASTX | 188 | 6.00E−18 | 63 | chlorophyll a/b-binding protein F24G24.140 - |
| | | | | | | | Arabidopsis thaliana |
| | | | | | | | >gi\|4538963\|emb\|CAB39787.1\| (AL049488) |
| | | | | | | | chlorophyll a/b-binding protein-like [Arabidopsis |
| | | | | | | | thaliana] |
| | | | | | | | >gi\|4741958\|gb\|AAD28776.1\|AF134129_1 |
| | | | | | | | (AF134129) Lhcb5 protein [Arabidopsis thaliana] |
| | | | | | | | >gi\|7 |
| 1821 | LIB3602-052-Q6-K1-E6 | 7443228 | BLASTX | 163 | 4.00E−11 | 59 | chlorophyll a/b-binding protein F24G24.140 - |
| | | | | | | | Arabidopsis thaliana |
| | | | | | | | >gi\|4538963\|emb\|CAB39787.1\| (AL049488) |
| | | | | | | | chlorophyll a/b-binding protein-like [Arabidopsis |
| | | | | | | | thaliana] |
| | | | | | | | >gi\|4741958\|gb\|AAD28776.1\|AF134129_1 |
| | | | | | | | (AF134129) Lhcb5 protein [Arabidopsis thaliana] |
| | | | | | | | >gi\|7 |
| 1822 | LIB3602-014-Q6-K1-H11 | 7442279 | BLASTX | 295 | 2.00E−26 | 60 | probable acyl carrier protein T8F5.6 - Arabidopsis |
| | | | | | | | thaliana >gi\|3335337\|gb\|AAC27139.1\| (AC004512) |
| | | | | | | | Similar to acyl carrier protein, mitochondrial |
| | | | | | | | precursor (ACP) NADH-ubiquinone oxidoreductase |
| | | | | | | | 9.6 KD subunit (MYACP-1), gb\|L23574 from |
| | | | | | | | A. thaliana. ESTs gb\| |
| 1823 | LIB3602-044-Q6-K1-E5 | 7442279 | BLASTX | 283 | 5.00E−25 | 67 | probable acyl carrier protein T8F5.6 - Arabidopsis |
| | | | | | | | thaliana >gi\|3335337\|gb\|AAC27139.1\| (AC004512) |
| | | | | | | | Similar to acyl carrier protein, mitochondrial |
| | | | | | | | precursor (ACP) NADH-ubiquinone oxidoreductase |
| | | | | | | | 9.6 KD subunit (MYACP-1), gb\|L23574 from |
| | | | | | | | A. thaliana. ESTs gb\| |
| 1824 | LIB3602-113-Q1-K1-E4 | 4558549 | BLASTX | 710 | 5.00E−75 | 71 | (AC007138) putative SecA-type chloroplast protein |
| | | | | | | | transport factor [Arabidopsis thaliana] |
| | | | | | | | >gi\|7268223\|emb\|CAB77750.1\| (AL161492) |
| | | | | | | | putative SecA-type chloroplast protein transport |
| | | | | | | | factor [Arabidopsis thaliana] |
| 1825 | LIB3602-114-Q1-K1-F7 | 4558549 | BLASTX | 700 | 8.00E−74 | 71 | (AC007138) putative SecA-type chloroplast protein |
| | | | | | | | transport factor [Arabidopsis thaliana] |
| | | | | | | | >gi\|7268223\|emb\|CAB77750.1\| (AL161492) |
| | | | | | | | putative SecA-type chloroplast protein transport |
| | | | | | | | factor [Arabidopsis thaliana] |
| 1826 | LIB3602-103-Q1-K1-F7 | 3107919 | BLASTX | 792 | 1.00E−84 | 95 | (AB013098) actin [Nannochloris bacillaris] |
| 1827 | LIB3602-109-Q1-K1-G10 | 3107919 | BLASTX | 672 | 1.00E−70 | 90 | (AB013098) actin [Nannochloris bacillaris] |
| 1828 | LIB3602-084-Q6-K1-H9 | 6984130 | BLASTX | 688 | 4.00E−73 | 82 | (AF227620) 60S ribosomal protein L10 [Euphorbia |
| | | | | | | | esula] |
| 1829 | LIB3602-007-Q1-K1-G2 | 6984130 | BLASTX | 264 | 4.00E−46 | 94 | (AF227620) 60S ribosomal protein L10 [Euphorbia |
| | | | | | | | esula] |
| 1830 | LIB3602-072-Q1-K1-A8 | 3122234 | BLASTX | 477 | 1.00E−47 | 63 | EUKARYOTIC TRANSLATION INITIATION |
| | | | | | | | FACTOR 2 BETA SUBUNIT (EIF-2-BETA) (P38) |
| | | | | | | | >gi\|7450843\|pir\|\|T06992 translation initiation factor |
| | | | | | | | eIF-2 beta chain - wheat |
| | | | | | | | >gi\|2306768\|gb\|AAB65774.1\| (U87163) eIF-2 beta |
| | | | | | | | subunit [Triticum aestivum] |
| 1831 | LIB3602-109-Q1-K1-D12 | 3122234 | BLASTX | 210 | 8.00E−31 | 59 | EUKARYOTIC TRANSLATION INITIATION |
| | | | | | | | FACTOR 2 BETA SUBUNIT (EIF-2-BETA) (P38) |
| | | | | | | | >gi\|7450843\|pir\|\|T06992 translation initiation factor |
| | | | | | | | eIF-2 beta chain - wheat |
| | | | | | | | >gi\|2306768\|gb\|AAB65774.1\| (U87163) eIF-2 beta |
| | | | | | | | subunit [Triticum aestivum] |
| 1832 | LIB3602-034-Q6-K1-C7 | 6523090 | BLASTX | 296 | 1.00E−26 | 71 | (AL133315) transcription factor Hap5a |
| | | | | | | | [Arabidopsis thaliana] |
| 1833 | LIB3602-035-Q1-K1-C7 | 6523090 | BLASTX | 291 | 5.00E−26 | 71 | (AL133315) transcription factor Hap5a |
| | | | | | | | [Arabidopsis thaliana] |
| 1834 | LIB3602-027-Q6-K1-E5 | 3122707 | BLASTX | 292 | 4.00E−26 | 58 | 50S RIBOSOMAL PROTEIN L24 |
| | | | | | | | >gi\|2446901\|dbj\|BAA22460.1\| (AB000111) 50S |
| | | | | | | | ribosomal protein L24 [Synechococcus sp.] |
| 1835 | LIB3602-109-Q1-K1-B7 | 3122707 | BLASTX | 246 | 9.00E−21 | 58 | 50S RIBOSOMAL PROTEIN L24 |
| | | | | | | | >gi\|2446901\|dbj\|BAA22460.1\| (AB000111) 50S |
| | | | | | | | ribosomal protein L24 [Synechococcus sp.] |
| 1836 | LIB3602-062-Q6-K1-B2 | 1173071 | BLASTX | 181 | 3.00E−13 | 41 | 60S ACIDIC RIBOSOMAL PROTEIN P2 (MINOR |
| | | | | | | | ALLERGEN ALT A 6) (ALT A VI) |
| | | | | | | | >gi\|1085614\|pir\|\|S43109 acidic ribosomal protein |
| | | | | | | | P2 - Alternaria alternata |
| | | | | | | | >gi\|467617\|emb\|CAA55066.1\| (X78222) minor |
| | | | | | | | allergen, ribosomal protein [Alternaria alternata] |
| 1837 | LIB3602-057-Q6-K1-D10 | 1173071 | BLASTX | 157 | 2.00E−10 | 40 | 60S ACIDIC RIBOSOMAL PROTEIN P2 (MINOR |
| | | | | | | | ALLERGEN ALT A 6) (ALT A VI) |
| | | | | | | | >gi\|1085614\|pir\|\|S43109 acidic ribosomal protein |
| | | | | | | | P2 - Alternaria alternata |
| | | | | | | | >gi\|467617\|emb\|CAA55066.1\| (X78222) minor |
| | | | | | | | allergen, ribosomal protein [Alternaria alternata] |
| 1838 | LIB3602-010-Q6-K1-G10 | 1173070 | BLASTX | 201 | 2.00E−15 | 52 | 50S RIBOSOMAL PROTEIN L9 |
| | | | | | | | >gi\|485508\|pir\|\|S33614 ribosomal protein L9 - |
| | | | | | | | Synechocystis sp. (strain PCC 6803) |
| | | | | | | | >gi\|217101\|dbj\|BAA38819.1\| (D10716) 50S |
| | | | | | | | ribosomal protein L9 [Synechocystis sp.] |
| | | | | | | | >gi\|1653258\|dbj\|BAA18173.1\| (D90912) 50S |
| | | | | | | | ribosomal protein L9 [Syne |
| 1839 | LIB3602-011-Q6-K1-B7 | 1173070 | BLASTX | 176 | 2.00E−12 | 38 | 50S RIBOSOMAL PROTEIN L9 |
| | | | | | | | >gi\|485508\|pir\|\|S33614 ribosomal protein L9 - |
| | | | | | | | Synechocystis sp. (strain PCC 6803) |
| | | | | | | | >gi\|217101\|dbj\|BAA38819.1\| (D10716) 50S |
| | | | | | | | ribosomal protein L9 [Synechocystis sp.] |
| | | | | | | | >gi\|1653258\|dbj\|BAA18173.1\| (D90912) 50S |
| | | | | | | | ribosomal protein L9 [Syne |
| 1840 | LIB3602-056-Q6-K1-C1 | 2765356 | BLASTX | 556 | 4.00E−57 | 80 | (Y13865) chlorophyll a/b-binding protein [Beta |
| | | | | | | | vulgaris] |
| 1841 | LIB3602-013-Q6-K1-E5 | 2765356 | BLASTX | 535 | 1.00E−54 | 80 | (Y13865) chlorophyll a/b-binding protein [Beta |
| | | | | | | | vulgaris] |
| 1842 | LIB3602-076-Q6-K6-D3 | 3024019 | BLASTX | 586 | 2.00E−60 | 74 | INITIATION FACTOR 5A (EIF-5A) (EIF-4D) |
| | | | | | | | >gi\|2225879\|dbj\|BAA20876.1\| (AB004823) |
| | | | | | | | eukaryotic initiation factor 5A2 [Solanum |
| | | | | | | | tuberosum] >gi\|2225887\|dbj\|BAA20880.1\| |
| | | | | | | | (AB004827) eukaryotic initiation factor 5A1 |
| | | | | | | | [Solanum tuberosum] |
| 1843 | LIB3602-061-Q6-K1-F3 | 3024019 | BLASTX | 353 | 3.00E−33 | 66 | INITIATION FACTOR 5A (EIF-5A) (EIF-4D) |
| | | | | | | | >gi\|2225879\|dbj\|BAA20876.1\| (AB004823) |
| | | | | | | | eukaryotic initiation factor 5A2 [Solanum |
| | | | | | | | tuberosum] >gi\|2225887\|dbj\|BAA20880.1\| |
| | | | | | | | (AB004827) eukaryotic initiation factor 5A1 |
| | | | | | | | [Solanum tuberosum] |
| 1844 | LIB3602-007-Q1-K1-E10 | 543867 | BLASTX | 431 | 3.00E−42 | 52 | ATP SYNTHASE GAMMA CHAIN, |
| | | | | | | | MITOCHONDRIAL PRECURSOR |
| | | | | | | | >gi\|1076684\|pir\|\|A47493 H+-transporting ATP |
| | | | | | | | synthase (EC 3.6.1.34) gamma chain precursor, |
| | | | | | | | mitochondrial - sweet potato |
| | | | | | | | >gi\|303626\|dbj\|BAA03526.1\| (D14699) F1-ATPase |
| | | | | | | | gammma subunit [Ipomoea batatas] |
| 1845 | LIB3602-072-Q1-K1-D4 | 543867 | BLASTX | 385 | 6.00E−37 | 60 | ATP SYNTHASE GAMMA CHAIN, |
| | | | | | | | MITOCHONDRIAL PRECURSOR |
| | | | | | | | >gi\|1076684\|pir\|\|A47493 H+-transporting ATP |
| | | | | | | | synthase (EC 3.6.1.34) gamma chain precursor, |
| | | | | | | | mitochondrial - sweet potato |
| | | | | | | | >gi\|303626\|dbj\|BAA03526.1\| (D14699) F1-ATPase |
| | | | | | | | gammma subunit [Ipomoea batatas] |
| 1846 | LIB3602-032-Q6-K1-F1 | 3293547 | BLASTX | 298 | 8.00E−27 | 46 | (AF072709) putative oxidoreductase [Streptomyces |
| | | | | | | | lividans] |
| 1847 | LIB3602-023-Q6-K1-F1 | 3293547 | BLASTX | 215 | 5.00E−17 | 33 | (AF072709) putative oxidoreductase [Streptomyces |
| | | | | | | | lividans] |
| 1848 | LIB3602-032-Q6-K1-B4 | 7024455 | BLASTX | 588 | 7.00E−61 | 77 | (AB034696) vacuolar-pyrophosphatase like protein |
| | | | | | | | [Arabidopsis thaliana] |
| 1849 | LIB3602-050-Q6-K1-C4 | 7024455 | BLASTX | 238 | 7.00E−20 | 77 | (AB034696) vacuolar-pyrophosphatase like protein |
| | | | | | | | [Arabidopsis thaliana] |
| 1850 | LIB3602-118-Q1-K1-F12 | 5052206 | BLASTX | 216 | 4.00E−17 | 38 | (AF100198) putative NAD(P)H steroid |
| | | | | | | | dehydrogenase [Mus musculus] |
| | | | | | | | >gi\|7619723\|emb\|CAA15948.2\| (AL021127) |
| | | | | | | | NAD(P)H steroid dehydrogenase [Mus musculus] |
| 1851 | LIB3602-105-Q1-K1-C4 | 5052206 | BLASTX | 211 | 2.00E−16 | 38 | (AF100198) putative NAD(P)H steroid |
| | | | | | | | dehydrogenase [Mus musculus] |
| | | | | | | | >gi\|7619723\|emb\|CAA15948.2\| (AL021127) |
| | | | | | | | NAD(P)H steroid dehydrogenase [Mus musculus] |
| 1852 | LIB3602-070-Q1-K1-F7 | 7297921 | BLASTX | 674 | 9.00E−71 | 63 | (AE003635) CG5353 gene product [alt 1] |
| | | | | | | | [Drosophila melanogaster] |
| | | | | | | | >gi\|7297922\|gb\|AAF53167.1\| (AE003635) CG5353 |
| | | | | | | | gene product [alt 2] [Drosophila melanogaster] |
| 1853 | LIB3602-012-Q6-K1-B7 | 7297921 | BLASTX | 493 | 1.00E−49 | 56 | (AE003635) CG5353 gene product [alt 1] |
| | | | | | | | [Drosophila melanogaster] |
| | | | | | | | >gi\|7297922\|gb\|AAF53167.1\| (AE003635) CG5353 |
| | | | | | | | gene product [alt 2] [Drosophila melanogaster] |
| 1854 | LIB3602-058-Q6-K1-H3 | 2129459 | BLASTX | 612 | 1.00E−63 | 76 | chaperonin 60 alpha chain precursor, chloroplast - |
| | | | | | | | Chlamydomonas reinhardtii |
| 1855 | LIB3602-017-Q6-K1-G3 | 2129459 | BLASTX | 214 | 7.00E−17 | 62 | chaperonin 60 alpha chain precursor, chloroplast - |
| | | | | | | | Chlamydomonas reinhardtii |
| 1856 | LIB3602-076-Q6-K6-G5 | 3776005 | BLASTX | 386 | 5.00E−37 | 83 | (AJ010466) RNA helicase [Arabidopsis thaliana] |
| 1857 | LIB3602-076-Q6-K6-H1 | 3776005 | BLASTX | 149 | 2.00E−09 | 83 | (AJ010466) RNA helicase [Arabidopsis thaliana] |
| 1858 | LIB3602-066-Q1-K6-G2 | 2911276 | BLASTX | 174 | 4.00E−12 | 70 | (U63631) LMW heat shock protein [Fragaria x |
| | | | | | | | ananassa] |
| 1859 | LIB3602-106-Q1-K1-B1 | 2911276 | BLASTX | 174 | 4.00E−12 | 70 | (U63631) LMW heat shock protein [Fragaria x |
| | | | | | | | ananassa] |
| 1860 | LIB3602-100-Q1-K1-G3 | 7484780 | BLASTX | 447 | 4.00E−44 | 46 | 26S proteasome regulatory particle chain RPN7 |
| | | | | | | | homolog F6I7.30 - Arabidopsis thaliana |
| | | | | | | | >gi\|4678261\|emb\|CAB41122.1\| (AL049657) |
| | | | | | | | putative proteasome regulatory subunit [Arabidopsis |
| | | | | | | | thaliana] >gi\|7269333\|emb\|CAB79392.1\| |
| | | | | | | | (AL161562) putative proteasome regulatory |
| 1861 | LIB3602-106-Q1-K1-G3 | 7484780 | BLASTX | 419 | 7.00E−41 | 45 | 26S proteasome regulatory particle chain RPN7 |
| | | | | | | | homolog F6I7.30 - Arabidopsis thaliana |
| | | | | | | | >gi\|4678261\|emb\|CAB41122.1\| (AL049657) |
| | | | | | | | putative proteasome regulatory subunit [Arabidopsis |
| | | | | | | | thaliana] >gi\|7269333\|emb\|CAB79392.1\| |
| | | | | | | | (AL161562) putative proteasome regulatory |
| 1862 | LIB3602-100-Q1-K1-H7 | 2494116 | BLASTX | 352 | 4.00E−33 | 52 | (AC002376) Similar to Synechocystis hypothetical |
| | | | | | | | protein (gb\|D90915). [Arabidopsis thaliana] |
| 1863 | LIB3602-106-Q1-K1-H7 | 2494116 | BLASTX | 297 | 1.00E−27 | 51 | (AC002376) Similar to Synechocystis hypothetical |
| | | | | | | | protein (gb\|D90915). [Arabidopsis thaliana] |
| 1864 | LIB3602-026-Q6-K1-H2 | 7262687 | BLASTX | 147 | 5.00E−09 | 45 | (AC012188) Contains similarity to an unknown |
| | | | | | | | protein from Arabidopsis thaliana gb\|AC004136.2. |
| | | | | | | | ESTs gb\|Z47683, gb\|Z47682, gb\|AA597850, |
| | | | | | | | gb\|Z29736, gb\|Z29735, gb\|AA042623 come from |
| | | | | | | | this gene |
| 1865 | LIB3602-036-Q6-K1-C9 | 7262687 | BLASTX | 147 | 6.00E−09 | 45 | (AC012188) Contains similarity to an unknown |
| | | | | | | | protein from Arabidopsis thaliana gb\|AC004136.2. |
| | | | | | | | ESTs gb\|Z47683, gb\|Z47682, gb\|AA597850, |
| | | | | | | | gb\|Z29736, gb\|Z29735, gb\|AA042623 come from |
| | | | | | | | this gene |
| 1866 | LIB3602-084-Q6-K1-E12 | 4049408 | BLASTX | 278 | 2.00E−24 | 52 | (Y10224) L-ascorbate oxidase [Cucumis melo] |
| 1867 | LIB3602-095-Q6-K6-B2 | 4049408 | BLASTX | 166 | 3.00E−11 | 54 | (Y10224) L-ascorbate oxidase [Cucumis melo] |
| 1868 | LIB3602-034-Q6-K1-E8 | 5921663 | BLASTX | 745 | 4.00E−79 | 76 | (AF162279) 10-formyltetrahydrofolate synthetase |
| | | | | | | | [Arabidopsis thaliana] |
| 1869 | LIB3602-035-Q1-K1-E8 | 5921663 | BLASTX | 665 | 1.00E−71 | 76 | (AF162279) 10-formyltetrahydrofolate synthetase |
| | | | | | | | [Arabidopsis thaliana] |
| 1870 | LIB3602-003-Q1-K1-G9 | 6010630 | BLASTX | 184 | 2.00E−13 | 41 | (AF179401) TspO [Sinorhizobium meliloti] |
| 1871 | LIB3602-050-Q6-K1-E7 | 6010630 | BLASTX | 167 | 2.00E−11 | 48 | (AF179401) TspO [Sinorhizobium meliloti] |
| 1872 | LIB3602-114-Q1-K1-F8 | 280386 | BLASTX | 318 | 5.00E−43 | 90 | ubiquitin/ribosomal protein CEP52 - Arabidopsis |
| | | | | | | | thaliana >gi\|166930\|gb\|AAA32904.1\| (J05507) |
| | | | | | | | ubiquitin extension protein (UBQ1) [Arabidopsis |
| | | | | | | | thaliana] >gi\|166932\|gb\|AAA32905.1\| (J05508) |
| | | | | | | | ubiquitin extension protein (UBQ2) [Arabidopsis |
| | | | | | | | thaliana] >gi\|488626 |
| 1873 | LIB3602-116-Q1-K1-C3 | 280386 | BLASTX | 170 | 3.00E−15 | 60 | ubiquitin/ribosomal protein CEP52 - Arabidopsis |
| | | | | | | | thaliana >gi\|166930\|gb\|AAA32904.1\| (J05507) |
| | | | | | | | ubiquitin extension protein (UBQ1) [Arabidopsis |
| | | | | | | | thaliana] >gi\|166932\|gb\|AAA32905.1\| (J05508) |
| | | | | | | | ubiquitin extension protein (UBQ2) [Arabidopsis |
| | | | | | | | thaliana] >gi\|488626 |
| 1874 | LIB3602-013-Q6-K1-G6 | 82080 | BLASTX | 445 | 4.00E−44 | 66 | chlorophyll a/b-binding protein type III precursor - |
| | | | | | | | tomato >gi\|226872\|prf\|\|1609235A chlorophyll a/b |
| | | | | | | | binding protein [Lycopersicon esculentum] |
| 1875 | LIB3602-058-Q6-K1-F8 | 82080 | BLASTX | 352 | 4.00E−33 | 69 | chlorophyll a/b-binding protein type III precursor - |
| | | | | | | | tomato >gi\|226872\|prf\|\|1609235A chlorophyll a/b |
| | | | | | | | binding protein [Lycopersicon esculentum] |
| 1876 | LIB3602-067-Q1-K1-F5 | 7437318 | BLASTX | 649 | 8.00E−68 | 73 | peptidylprolyl isomerase (EC 5.2.1.8) 1 - |
| | | | | | | | Chlamydomonas reinhardtii |
| | | | | | | | >gi\|2959712\|gb\|AAC05639.1\| (AF052206) |
| | | | | | | | cyclophilin 1 [Chlamydomonas reinhardtii] |
| 1877 | LIB3602-085-Q6-K1-C10 | 7437318 | BLASTX | 642 | 5.00E−67 | 72 | peptidylprolyl isomerase (EC 5.2.1.8) 1 - |
| | | | | | | | Chlamydomonas reinhardtii |
| | | | | | | | >gi\|2959712\|gb\|AAC05639.1\| (AF052206) |
| | | | | | | | cyclophilin 1 [Chlamydomonas reinhardtii] |
| 1878 | LIB3602-093-Q6-K6-G3 | 1173194 | BLASTX | 242 | 2.00E−23 | 59 | 30S RIBOSOMAL PROTEIN S13, |
| | | | | | | | CHLOROPLAST PRECURSOR (CS13) |
| | | | | | | | >gi\|2119093\|pir\|\|S59594 ribosomal protein S13 |
| | | | | | | | precursor, choroplast - Arabidopsis thaliana |
| | | | | | | | >gi\|16767\|emb\|CAA79013.1\| (Z17611) chloroplast |
| | | | | | | | 30S ribosomal protein S13 [Arabidopsis thaliana] |
| | | | | | | | >gi\|662869\| |
| 1879 | LIB3602-048-Q6-K1QA-H7 | 1173194 | BLASTX | 205 | 5.00E−16 | 62 | 30S RIBOSOMAL PROTEIN S13, |
| | | | | | | | CHLOROPLAST PRECURSOR (CS13) |
| | | | | | | | >gi\|2119093\|pir\|\|S59594 ribosomal protein S13 |
| | | | | | | | precursor, choroplast - Arabidopsis thaliana |
| | | | | | | | >gi\|16767\|emb\|CAA79013.1\| (Z17611) chloroplast |
| | | | | | | | 30S ribosomal protein S13 [Arabidopsis thaliana] |
| | | | | | | | >gi\|662869\| |
| 1880 | LIB3602-055-Q6-K1-C3 | 6822076 | BLASTX | 382 | 9.00E−37 | 58 | (AL132957) synaptobrevin-like protein |
| | | | | | | | [Arabidopsis thaliana] |
| 1881 | LIB3602-100-Q1-K1-A11 | 6822076 | BLASTX | 322 | 1.00E−29 | 50 | (AL132957) synaptobrevin-like protein |
| | | | | | | | [Arabidopsis thaliana] |
| 1882 | LIB3602-083-Q6-K6-H9 | 7439994 | BLASTX | 171 | 8.00E−12 | 37 | glycine-rich RNA-binding protein - garden pea |
| | | | | | | | >gi\|1778374\|gb\|AAB71417.1\| (U81287) glycine- |
| | | | | | | | rich RNA-binding protein PsGRBP [Pisum sativum] |
| 1883 | LIB3602-109-Q1-K1-H9 | 7439994 | BLASTX | 146 | 7.00E−09 | 36 | glycine-rich RNA-binding protein - garden pea |
| | | | | | | | >gi\|1778374\|gb\|AAB71417.1\| (U81287) glycine- |
| | | | | | | | rich RNA-binding protein PsGRBP [Pisum sativum] |
| 1884 | LIB3602-043-Q6-K1-F3 | 6175164 | BLASTX | 376 | 6.00E−36 | 60 | (AC011437) putative tryptophanyl-tRNA synthetase |
| | | | | | | | [Arabidopsis thaliana] |
| 1885 | LIB3602-112-Q1-K1-E4 | 6175164 | BLASTX | 148 | 3.00E−17 | 42 | (AC011437) putative tryptophanyl-tRNA synthetase |
| | | | | | | | [Arabidopsis thaliana] |
| 1886 | LIB3602-049-Q6-K1-C5 | 7381225 | BLASTX | 240 | 6.00E−20 | 49 | (AF139187) root border cell-specific protein [Pisum |
| | | | | | | | sativum] |
| 1887 | LIB3602-048-Q6-K1QA-E4 | 7381225 | BLASTX | 212 | 9.00E−17 | 53 | (AF139187) root border cell-specific protein [Pisum |
| | | | | | | | sativum] |
| 1888 | LIB3602-118-Q1-K1-F9 | 7492651 | BLASTX | 154 | 5.00E−10 | 83 | probable histone h3 variant - fission yeast |
| | | | | | | | (Schizosaccharomyces pombe) |
| | | | | | | | >gi\|5531479\|emb\|CAB50980.1\| (AL096851) |
| | | | | | | | probable histone h3 variant [Schizosaccharomyces |
| | | | | | | | pombe] >gi\|7630185\|dbj\|BAA94760.1\| (AB041724) |
| | | | | | | | CENP-A-like protein SpCENP-A |
| | | | | | | | [Schizosaccharomyces |
| 1889 | LIB3602-095-Q6-K6-E8 | 7492651 | BLASTX | 149 | 3.00E−09 | 80 | probable histone h3 variant - fission yeast |
| | | | | | | | (Schizosaccharomyces pombe) |
| | | | | | | | >gi\|5531479\|emb\|CAB50980.1\| (AL096851) |
| | | | | | | | probable histone h3 variant [Schizosaccharomyces |
| | | | | | | | pombe] >gi\|7630185\|dbj\|BAA94760.1\| (AB041724) |
| | | | | | | | CENP-A-like protein SpCENP-A |
| | | | | | | | [Schizosaccharomyces |
| 1890 | LIB3602-034-Q6-K1-F8 | 485512 | BLASTX | 308 | 6.00E−28 | 63 | glutathione peroxidase (EC 1.11.1.9) - sweet orange |
| 1891 | LIB3602-035-Q1-K1-F8 | 485512 | BLASTX | 308 | 7.00E−28 | 63 | glutathione peroxidase (EC 1.11.1.9) - sweet orange |
| 1892 | LIB3602-068-Q1-K1-H7 | 7438196 | BLASTX | 243 | 3.00E−43 | 74 | probable sulfate adenylyltransferase (EC 2.7.7.4) |
| | | | | | | | ATS1 - Chlamydomonas reinhardtii |
| | | | | | | | >gi\|1336213\|gb\|AAB01234.1\| (U57088) ATP |
| | | | | | | | sulfurylase Ats1 [Chlamydomonas reinhardtii] |
| 1893 | LIB3602-008-Q6-K1-D8 | 7438196 | BLASTX | 418 | 7.00E−41 | 60 | probable sulfate adenylyltransferase (EC 2.7.7.4) |
| | | | | | | | ATS1 - Chlamydomonas reinhardtii |
| | | | | | | | >gi\|1336213\|gb\|AAB01234.1\| (U57088) ATP |
| | | | | | | | sulfurylase Ats1 [Chlamydomonas reinhardtii] |
| 1894 | LIB3602-036-Q6-K1-C10 | 7451353 | BLASTX | 683 | 1.00E−71 | 75 | SAR DNA-binding protein-1 - garden pea |
| | | | | | | | >gi\|3132696\|gb\|AAC16330.1\| (AF061962) SAR |
| | | | | | | | DNA-binding protein-1 [Pisum sativum] |
| 1895 | LIB3602-058-Q6-K1-G12 | 7451353 | BLASTX | 307 | 3.00E−28 | 68 | SAR DNA-binding protein-1 - garden pea |
| | | | | | | | >gi\|3132696\|gb\|AAC16330.1\| (AF061962) SAR |
| | | | | | | | DNA-binding protein-1 [Pisum sativum] |
| 1896 | LIB3602-017-Q6-K1-F7 | 6272283 | BLASTX | 468 | 1.00E−46 | 62 | (AJ250770) plastidial phosphoglucomutase [Pisum |
| | | | | | | | sativum] |
| 1897 | LIB3602-101-Q1-K1-H8 | 6272283 | BLASTX | 451 | 9.00E−45 | 57 | (AJ250770) plastidial phosphoglucomutase [Pisum |
| | | | | | | | sativum] |
| 1898 | LIB3602-081-Q6-K6-G3 | 4567256 | BLASTX | 164 | 2.00E−11 | 90 | (AC007070) 60S ribosomal protein L39 |
| | | | | | | | [Arabidopsis thaliana] |
| 1899 | LIB3602-082-Q6-K6-B3 | 4567256 | BLASTX | 164 | 2.00E−11 | 90 | (AC007070) 60S ribosomal protein L39 |
| | | | | | | | [Arabidopsis thaliana] |
| 1900 | LIB3602-047-Q6-K1-E6 | 401237 | BLASTX | 489 | 3.00E−49 | 60 | UBIQUITIN-ACTIVATING ENZYME E1 2 |
| | | | | | | | >gi\|170684\|gb\|AAA34265.1\| (M90663) ubiquitin |
| | | | | | | | activating enyme [Triticum aestivum] |
| 1901 | LIB3602-090-Q6-K6-A1 | 401237 | BLASTX | 265 | 6.00E−23 | 64 | UBIQUITIN-ACTIVATING ENZYME E1 2 |
| | | | | | | | >gi\|170684\|gb\|AAA34265.1\| (M90663) ubiquitin |
| | | | | | | | activating enyme [Triticum aestivum] |
| 1902 | LIB3602-039-Q6-K1-A6 | 1065365 | BLASTX | 184 | 3.00E−13 | 57 | Alpha-1,4 Glycan-4-Glucanohydrolase (Alpha- |
| | | | | | | | Amylase, High Pi Isozyme (Amy2)) (E.C.3.2.1.1) |
| | | | | | | | >gi\|4699831\|pdb\|1AVA\|A Chain A, Amy2BASI |
| | | | | | | | PROTEIN-Protein Complex From Barley Seed |
| | | | | | | | >gi\|4699832\|pdb\|1AVA\|B Chain B, Amy2BASI |
| | | | | | | | PROTEIN-Protein Complex From Barley Seed > |
| 1903 | LIB3602-050-Q6-K1-E8 | 1065365 | BLASTX | 180 | 3.00E−13 | 54 | Alpha-1,4 Glycan-4-Glucanohydrolase (Alpha- |
| | | | | | | | Amylase, High Pi Isozyme (Amy2)) (E.C.3.2.1.1) |
| | | | | | | | >gi\|4699831\|pdb\|1AVA\|A Chain A, Amy2BASI |
| | | | | | | | PROTEIN-Protein Complex From Barley Seed |
| | | | | | | | >gi\|4699832\|pdb\|1AVA\|B Chain B, Amy2BASI |
| | | | | | | | PROTEIN-Protein Complex From Barley Seed > |
| 1904 | LIB3602-094-Q6-K6-B2 | 595775 | BLASTX | 199 | 5.00E−15 | 36 | (U13869) lacZ alpha peptide [unidentified cloning |
| | | | | | | | vector] |
| 1905 | LIB3602-022-Q6-K1-H10 | 595775 | BLASTX | 150 | 1.00E−09 | 42 | (U13869) lacZ alpha peptide [unidentified cloning |
| | | | | | | | vector] |
| 1906 | LIB3602-065-Q1-K6-C4 | 6094085 | BLASTX | 697 | 2.00E−73 | 72 | 60S RIBOSOMAL PROTEIN L5 |
| | | | | | | | >gi\|7440744\|pir\|\|T08009 probable ribosomal protein |
| | | | | | | | L5 - green alga (Dunaliella salina) |
| | | | | | | | >gi\|2599104\|gb\|AAB84056.1\| (AF028833) 60S |
| | | | | | | | ribosomal protein [Dunaliella salina] |
| 1907 | LIB3602-080-Q6-K6-H9 | 6094085 | BLASTX | 489 | 4.00E−49 | 58 | 60S RIBOSOMAL PROTEIN L5 |
| | | | | | | | >gi\|7440744\|pir\|\|T08009 probable ribosomal protein |
| | | | | | | | L5 - green alga (Dunaliella salina) |
| | | | | | | | >gi\|2599104\|gb\|AAB84056.1\| (AF028833) 60S |
| | | | | | | | ribosomal protein [Dunaliella salina] |
| 1908 | LIB3602-018-Q6-K1-C4 | 4762038 | BLASTX | 190 | 5.00E−14 | 34 | (AL031909) probable surface antigen [Leishmania |
| | | | | | | | major] |
| 1909 | LIB3602-049-Q6-K1-D9 | 4762038 | BLASTX | 164 | 4.00E−11 | 31 | (AL031909) probable surface antigen [Leishmania |
| | | | | | | | major] |
| 1910 | LIB3602-092-Q6-K6-C5 | 6466950 | BLASTX | 824 | 2.00E−88 | 75 | (AC009176) putative ribulose-1,5-bisphosphate |
| | | | | | | | carboxylase/oxygenase small subunit N- |
| | | | | | | | methyltransferase I [Arabidopsis thaliana] |
| | | | | | | | >gi\|6648179\|gb\|AAF21177.1\|AC013483_1 |
| | | | | | | | (AC013483) putative ribulose-1,5-bisphosphate |
| | | | | | | | carboxylase/oxygenase small subunit N-methylt |
| 1911 | LIB3602-120-Q1-K1-C5 | 6466950 | BLASTX | 329 | 9.00E−31 | 72 | (AC009176) putative ribulose-1,5-bisphosphate |
| | | | | | | | carboxylase/oxygenase small subunit N- |
| | | | | | | | methyltransferase I [Arabidopsis thaliana] |
| | | | | | | | >gi\|6648179\|gb\|AAF21177.1\|AC013483_1 |
| | | | | | | | (AC013483) putative ribulose-1,5-bisphosphate |
| | | | | | | | carboxylase/oxygenase small subunit N-methylt |
| 1912 | LIB3602-057-Q6-K1-D6 | 129916 | BLASTX | 350 | 2.00E−50 | 51 | PHOSPHOGLYCERATE KINASE, CYTOSOLIC |
| | | | | | | | >gi\|66911\|pir\|\|TVWTGY phosphoglycerate kinase |
| | | | | | | | (EC 2.7.2.3), cytosolic - wheat |
| | | | | | | | >gi\|21835\|emb\|CAA33302.1\| (X15232) |
| | | | | | | | phosphoglycerate kinase (AA 1-401) [Triticum |
| | | | | | | | aestivum] |
| 1913 | LIB3602-032-Q6-K1-F5 | 129916 | BLASTX | 466 | 2.00E−46 | 64 | PHOSPHOGLYCERATE KINASE, CYTOSOLIC |
| | | | | | | | >gi\|66911\|pir\|\|TVWTGY phosphoglycerate kinase |
| | | | | | | | (EC 2.7.2.3), cytosolic - wheat |
| | | | | | | | >gi\|21835\|emb\|CAA33302.1\| (X15232) |
| | | | | | | | phosphoglycerate kinase (AA 1-401) [Triticum |
| | | | | | | | aestivum] |
| 1914 | LIB3602-041-Q6-K1-A5 | 4586054 | BLASTX | 320 | 2.00E−29 | 41 | (AC007020) unknown protein [Arabidopsis |
| | | | | | | | thaliana] |
| 1915 | LIB3602-013-Q6-K1-H3 | 4586054 | BLASTX | 191 | 2.00E−14 | 45 | (AC007020) unknown protein [Arabidopsis |
| | | | | | | | thaliana] |
| 1916 | LIB3602-092-Q6-K6-H3 | 7471624 | BLASTX | 163 | 8.00E−11 | 58 | conserved hypothetical protein - Deinococcus |
| | | | | | | | radiodurans (strain R1) |
| | | | | | | | >gi\|6458170\|gb\|AAF10062.1\|AE001907_8 |
| | | | | | | | (AE001907) conserved hypothetical protein |
| | | | | | | | [Deinococcus radiodurans] |
| 1917 | LIB3602-113-Q1-K1-H5 | 7471624 | BLASTX | 163 | 8.00E−11 | 58 | conserved hypothetical protein - Deinococcus |
| | | | | | | | radiodurans (strain R1) |
| | | | | | | | >gi\|6458170\|gb\|AAF10062.1\|AE001907_8 |
| | | | | | | | (AE001907) conserved hypothetical protein |
| | | | | | | | [Deinococcus radiodurans] |
| 1918 | LIB3602-100-Q1-K1-H1 | 5734741 | BLASTX | 434 | 9.00E−43 | 54 | (AC007651) Similar to Ubiquitin Conjugating |
| | | | | | | | Enzyme [Arabidopsis thaliana] |
| 1919 | LIB3602-105-Q1-K1-H10 | 5734741 | BLASTX | 282 | 8.00E−25 | 39 | (AC007651) Similar to Ubiquitin Conjugating |
| | | | | | | | Enzyme [Arabidopsis thaliana] |
| 1920 | LIB3602-036-Q6-K1-H6 | 7431451 | BLASTX | 375 | 1.00E−35 | 57 | rf2 nuclear restorer protein - maize |
| | | | | | | | >gi\|1421730\|gb\|AAC49371.1\| (U43082) RF2 [Zea |
| | | | | | | | mays] |
| 1921 | LIB3602-030-Q6-K1-H10 | 7431451 | BLASTX | 342 | 5.00E−32 | 57 | rf2 nuclear restorer protein - maize |
| | | | | | | | >gi\|1421730\|gb\|AAC49371.1\| (U43082) RF2 [Zea |
| | | | | | | | mays] |
| 1922 | LIB3602-043-Q6-K1-C2 | 7445551 | BLASTX | 203 | 2.00E−15 | 55 | ABC-type transport protein T18B16.180 - |
| | | | | | | | Arabidopsis thaliana |
| | | | | | | | >gi\|2828296\|emb\|CAA16710.1\| (AL021687) RNase |
| | | | | | | | L inhibitor-like protein [Arabidopsis thaliana] |
| | | | | | | | >gi\|17268716\|emb\|CAB78923.1\| (AL161550) RNase |
| | | | | | | | L inhibitor-like protein [Arabidopsis thaliana] |
| 1923 | LIB3602-061-Q6-K1-B4 | 7445551 | BLASTX | 196 | 7.00E−15 | 78 | ABC-type transport protein T18B16.180 - |
| | | | | | | | Arabidopsis thaliana |
| | | | | | | | >gi\|2828296\|emb\|CAA16710.1\| (AL021687) RNase |
| | | | | | | | L inhibitor-like protein [Arabidopsis thaliana] |
| | | | | | | | >gi\|7268716\|emb\|CAB78923.1\| (AL161550) RNase |
| | | | | | | | L inhibitor-like protein [Arabidopsis thaliana] |
| 1924 | LIB3602-070-Q1-K1-C5 | 6671365 | BLASTX | 639 | 1.00E−66 | 61 | (AF216497) P-glycoprotein [Gossypium hirsutum] |
| 1925 | LIB3602-021-Q6-K1-C10 | 6671365 | BLASTX | 161 | 4.00E−11 | 63 | (AF216497) P-glycoprotein [Gossypium hirsutum] |
| 1926 | LIB3602-063-Q1-K6-F8 | 7413536 | BLASTX | 194 | 2.00E−14 | 41 | (AL162972) putative protein [Arabidopsis thaliana] |
| 1927 | LIB3602-072-Q1-K1-G7 | 7413536 | BLASTX | 194 | 2.00E−14 | 41 | (AL162972) putative protein [Arabidopsis thaliana] |
| 1928 | LIB3602-087-Q6-K1-E5 | 7486255 | BLASTX | 293 | 4.00E−26 | 41 | hypothetical protein F27G19.20 - Arabidopsis |
| | | | | | | | thaliana >gi\|4972067\|emb\|CAB43874.1\| |
| | | | | | | | (AL078467) putative protei [Arabidopsis thaliana] |
| | | | | | | | >gi\|7269596\|emb\|CAB81392.1\| (AL161571) |
| | | | | | | | putative protein [Arabidopsis thaliana] |
| 1929 | LIB3602-029-Q6-K1-H2 | 7486255 | BLASTX | 221 | 1.00E−17 | 32 | hypothetical protein F27G19.20 - Arabidopsis |
| | | | | | | | thaliana >gi\|4972067\|emb\|CAB43874.1\| |
| | | | | | | | (AL078467) putative protei [Arabidopsis thaliana] |
| | | | | | | | >gi\|7269596\|emb\|CAB81392.1\| (AL161571) |
| | | | | | | | putative protein [Arabidopsis thaliana] |
| 1930 | LIB3602-109-Q1-K1-C4 | 3261517 | BLASTN | 34 | 3.00E−09 | 97 | Mycobacterium tuberculosis H37Rv complete |
| | | | | | | | genome; segment 143/162 |
| 1931 | LIB3602-083-Q6-K6-C4 | 3261517 | BLASTN | 34 | 4.00E−09 | 97 | Mycobacterium tuberculosis H37Rv complete |
| | | | | | | | genome; segment 143/162 |
| 1932 | LIB3602-052-Q6-K1-E7 | 6539249 | BLASTX | 520 | 7.00E−53 | 69 | (AC011765) geranylgeranyl reductase [Arabidopsis |
| | | | | | | | thaliana] |
| 1933 | LIB3602-012-Q6-K1-H2 | 6539249 | BLASTX | 477 | 7.00E−48 | 65 | (AC011765) geranylgeranyl reductase [Arabidopsis |
| | | | | | | | thaliana] |
| 1934 | LIB3602-022-Q6-K1-D9 | 541954 | BLASTX | 301 | 3.00E−27 | 45 | ubiquitin/ribosomal protein S27a fusion protein - |
| | | | | | | | white lupine >gi\|438111\|emb\|CAA80334.1\| |
| | | | | | | | (Z22613) ubiquitin extension protein [Lupinus |
| | | | | | | | albus] |
| 1935 | LIB3602-037-Q6-K1-B12 | 541954 | BLASTX | 186 | 5.00E−14 | 43 | ubiquitin/ribosomal protein S27a fusion protein - |
| | | | | | | | white lupine >gi\|438111\|emb\|CAA80334.1\| |
| | | | | | | | (Z22613) ubiquitin extension protein [Lupinus |
| | | | | | | | albus] |
| 1936 | LIB3602-073-Q1-K1-H5 | 6091731 | BLASTX | 489 | 5.00E−49 | 48 | (AC010797) hypothetical protein [Arabidopsis |
| | | | | | | | thaliana] |
| 1937 | LIB3602-102-Q1-K1-F5 | 6091731 | BLASTX | 408 | 1.00E−39 | 48 | (AC010797) hypothetical protein [Arabidopsis |
| | | | | | | | thaliana] |
| 1938 | LIB3602-108-Q1-K1-E3 | 730652 | BLASTX | 401 | 5.00E−55 | 81 | 40S RIBOSOMAL PROTEIN S2 (STRINGS OF |
| | | | | | | | PEARLS PROTEIN) >gi\|1085158\|pir\|\|S50325 |
| | | | | | | | ribosomal protein S2 - fruit fly (Drosophila |
| | | | | | | | melanogaster) >gi\|515971\|gb\|AAC34198.1\| |
| | | | | | | | (U01334) ribosomal protein S2 [Drosophila |
| | | | | | | | melanogaster] >gi\|515972\|gb\|AAA87053.1\| |
| | | | | | | | (U01335) ri |
| 1939 | LIB3602-083-Q6-K6-D3 | 730652 | BLASTX | 464 | 3.00E−46 | 66 | 40S RIBOSOMAL PROTEIN S2 (STRINGS OF |
| | | | | | | | PEARLS PROTEIN) >gi\|1085158\|pir\|\|S50325 |
| | | | | | | | ribosomal protein S2 - fruit fly (Drosophila |
| | | | | | | | melanogaster) >gi\|515971\|gb\|AAC34198.1\| |
| | | | | | | | (U01334) ribosomal protein S2 [Drosophila |
| | | | | | | | melanogaster] >gi\|515972\|gb\|AAA87053.1\| |
| | | | | | | | (U01335) ri |
| 1940 | LIB3602-108-Q1-K1-F7 | 464705 | BLASTX | 183 | 3.00E−15 | 88 | 40S RIBOSOMAL PROTEIN S13 |
| | | | | | | | >gi\|419802\|pir\|\|S30146 ribosomal protein S13, |
| | | | | | | | cytosolic - maize >gi\|288059\|emb\|CAA44311.1\| |
| | | | | | | | (X62455) cytoplasmatic ribosomal protein S13 |
| | | | | | | | [Zea mays] |
| 1941 | LIB3602-047-Q6-K1-A5 | 464705 | BLASTX | 187 | 5.00E−14 | 87 | 40S RIBOSOMAL PROTEIN S13 |
| | | | | | | | >gi\|419802\|pir\|\|S30146 ribosomal protein S13, |
| | | | | | | | cytosolic - maize >gi\|288059\|emb\|CAA44311.1\| |
| | | | | | | | (X62455) cytoplasmatic ribosomal protein S13 |
| | | | | | | | [Zea mays] |
| 1942 | LIB3602-009-Q6-K1-A9 | 1173055 | BLASTX | 474 | 1.00E−47 | 77 | 60S RIBOSOMAL PROTEIN L11 (L5) |
| | | | | | | | >gi\|541961\|pir\|\|S42497 ribosomal protein L11.e - |
| | | | | | | | alfalfa >gi\|1076504\|pir\|\|S51819 ribosomal protein |
| | | | | | | | L11, cytosolic - alfalfa |
| | | | | | | | >gi\|463252\|emb\|CAA55090.1\| (X78284) RL5 |
| | | | | | | | ribosomal protein [Medicago sativa] |
| 1943 | LIB3602-050-Q6-K1-F6 | 1173055 | BLASTX | 207 | 8.00E−17 | 70 | 60S RIBOSOMAL PROTEIN L11 (L5) |
| | | | | | | | >gi\|541961\|pir\|\|S42497 ribosomal protein L11.e - |
| | | | | | | | alfalfa >gi\|1076504\|pir\|\|S51819 ribosomal protein |
| | | | | | | | L11, cytosolic - alfalfa |
| | | | | | | | >gi\|463252\|emb\|CAA55090.1\| (X78284) RL5 |
| | | | | | | | ribosomal protein [Medicago sativa] |
| 1944 | LIB3602-006-Q1-K1-G11 | 567893 | BLASTX | 208 | 3.00E−16 | 43 | (L37382) beta-galactosidase-complementation |
| | | | | | | | protein [unidentified cloning vector] |
| 1945 | LIB3602-045-Q6-K1-G10 | 567893 | BLASTX | 204 | 8.00E−16 | 40 | (L37382) beta-galactosidase-complementation |
| | | | | | | | protein [unidentified cloning vector] |
| 1946 | LIB3602-026-Q6-K1-F10 | 7469882 | BLASTX | 181 | 5.00E−13 | 40 | hypothetical protein sll0408 - Synechocystis sp. |
| | | | | | | | (strain PCC 6803) >gi\|1001111\|dbj\|BAA10250.1\| |
| | | | | | | | (D64001) hypothetical protein [Synechocystis sp.] |
| 1947 | LIB3602-042-Q6-K1-H4 | 7469882 | BLASTX | 181 | 5.00E−13 | 40 | hypothetical protein sll0408 - Synechocystis sp. |
| | | | | | | | (strain PCC 6803) >gi\|1001111\|dbj\|BAA10250.1\| |
| | | | | | | | (D64001) hypothetical protein [Synechocystis sp.] |
| 1948 | LIB3602-072-Q1-K1-G2 | 7488914 | BLASTX | 681 | 1.00E−71 | 69 | embryogenic callus protein 181 - carrot (fragment) |
| | | | | | | | >gi\|3551247\|dbj\|BAA32822.1\| (AB012703) 181 |
| | | | | | | | [Daucus carota] |
| 1949 | LIB3602-065-Q1-K6-G2 | 7488914 | BLASTX | 457 | 2.00E−45 | 65 | embryogenic callus protein 181 - carrot (fragment) |
| | | | | | | | >gi\|3551247\|dbj\|BAA32822.1\| (AB012703) 181 |
| | | | | | | | [Daucus carota] |
| 1950 | LIB3602-081-Q6-K6-B1 | 6652882 | BLASTX | 501 | 1.00E−50 | 83 | (AF123392) 26S proteasome AAA-ATPase subunit |
| | | | | | | | RPT3 [Arabidopsis thaliana] |
| 1951 | LIB3602-115-Q1-K1-A4 | 6652882 | BLASTX | 326 | 5.00E−30 | 77 | (AF123392) 26S proteasome AAA-ATPase subunit |
| | | | | | | | RPT3 [Arabidopsis thaliana] |
| 1952 | LIB3602-070-Q1-K1-H10 | 7431102 | BLASTX | 277 | 3.00E−24 | 41 | plant metabolite dehydrogenase homolog ytbE - |
| | | | | | | | Bacillus subtilis >gi\|2293328\|gb\|AAC00406.1\| |
| | | | | | | | (AF008220) putative morphine dehydrogenase |
| | | | | | | | [Bacillus subtilis] >gi\|2635370\|emb\|CAB14865.1\| |
| | | | | | | | (Z99118) similar to plant metabolite dehydrogenase |
| | | | | | | | [Bacillus subtilis] |
| 1953 | LIB3602-102-Q1-K1-D9 | 7431102 | BLASTX | 256 | 7.00E−22 | 39 | plant metabolite dehydrogenase homolog ytbE - |
| | | | | | | | Bacillus subtilis >gi\|2293328\|gb\|AAC00406.1\| |
| | | | | | | | (AF008220) putative morphine dehydrogenase |
| | | | | | | | [Bacillus subtilis] >gi\|2635370\|emb\|CAB14865.1\| |
| | | | | | | | (Z99118) similar to plant metabolite dehydrogenase |
| | | | | | | | [Bacillus subtilis] |
| 1954 | LIB3602-013-Q6-K1-A5 | 2499807 | BLASTX | 426 | 1.00E−41 | 47 | GLUTAMATE 5-KINASE (GAMMA- |
| | | | | | | | GLUTAMYL KINASE) (GK) |
| | | | | | | | >gi\|7434577\|pir\|\|S75182 glutamate 5-kinase (EC |
| | | | | | | | 2.7.2.11) - Synechocystis sp. (strain PCC 6803) |
| | | | | | | | >gi\|1652172\|dbj\|BAA17096.1\| (D90903) glutamate |
| | | | | | | | 5-kinase [Synechocystis sp.] |
| 1955 | LIB3602-005-Q1-K1-H5 | 2499807 | BLASTX | 172 | 1.00E−12 | 51 | GLUTAMATE 5-KINASE (GAMMA- |
| | | | | | | | GLUTAMYL KINASE) (GK) |
| | | | | | | | >gi\|7434577\|pir\|\|S75182 glutamate 5-kinase (EC |
| | | | | | | | 2.7.2.11) - Synechocystis sp. (strain PCC 6803) |
| | | | | | | | >gi\|1652172\|dbj\|BAA17096.1\| (D90903) glutamate |
| | | | | | | | 5-kinase [Synechocystis sp.] |
| 1956 | LIB3602-056-Q6-K1-A2 | 7487663 | BLASTX | 152 | 1.00E−09 | 34 | hypothetical protein T4L20.280 - Arabidopsis |
| | | | | | | | thaliana >gi\|3096939\|emb\|CAA18849.1\| |
| | | | | | | | (AL023094) putative protein [Arabidopsis thaliana] |
| | | | | | | | >gi\|7270421\|emb\|CAB80187.1\| (AL161586) |
| | | | | | | | putative protein [Arabidopsis thaliana] |
| 1957 | LIB3602-039-Q6-K1-F8 | 7487663 | BLASTX | 150 | 2.00E−09 | 34 | hypothetical protein T4L20.280 - Arabidopsis |
| | | | | | | | thaliana >gi\|3096939\|emb\|CAA18849.1\| |
| | | | | | | | (AL023094) putative protein [Arabidopsis thaliana] |
| | | | | | | | >gi\|7270421\|emb\|CAB80187.1\| (AL161586) |
| | | | | | | | putative protein [Arabidopsis thaliana] |
| 1958 | LIB3602-018-Q6-K1-C1 | 4139215 | BLASTN | 49 | 2.00E−18 | 92 | Chlamydomonas reinhardtii light harvesting |
| | | | | | | | complex II protein precursor (Lhcb2) mRNA, |
| | | | | | | | complete cds |
| 1959 | LIB3602-061-Q6-K1-F8 | 4139215 | BLASTN | 44 | 2.00E−15 | 92 | Chlamydomonas reinhardtii light harvesting |
| | | | | | | | complex II protein precursor (Lhcb2) mRNA, |
| | | | | | | | complete cds |
| 1960 | LIB3602-018-Q6-K1-B11 | 6630729 | BLASTX | 298 | 6.00E−27 | 71 | (AL132958) putative protein [Arabidopsis thaliana] |
| 1961 | LIB3602-036-Q6-K1-A7 | 6630729 | BLASTX | 205 | 9.00E−16 | 47 | (AL132958) putative protein [Arabidopsis thaliana] |
| 1962 | LIB3602-101-Q1-K1-G11 | 7547401 | BLASTX | 202 | 6.00E−30 | 84 | (S66866) cytochrome c1 precursor [Solanum |
| | | | | | | | tuberosum] |
| 1963 | LIB3602-101-Q1-K1-G6 | 7547401 | BLASTX | 154 | 1.00E−21 | 72 | (S66866) cytochrome c1 precursor [Solanum |
| | | | | | | | tuberosum] |
| 1964 | LIB3602-070-Q1-K1-B3 | 7469888 | BLASTX | 335 | 5.00E−31 | 48 | hypothetical protein sll0424 - Synechocystis sp. |
| | | | | | | | (strain PCC 6803) >gi\|1653565\|dbj\|BAA18478.1\| |
| | | | | | | | (D90914) hypothetical protein [Synechocystis sp.] |
| 1965 | LIB3602-081-Q6-K6-F2 | 7469888 | BLASTX | 319 | 3.00E−29 | 43 | hypothetical protein sll0424 - Synechocystis sp. |
| | | | | | | | (strain PCC 6803) >gi\|1653565\|dbj\|BAA18478.1\| |
| | | | | | | | (D90914) hypothetical protein [Synechocystis sp.] |
| 1966 | LIB3602-061-Q6-K1-G1 | 7500560 | BLASTX | 306 | 1.00E−27 | 51 | hypothetical protein F35G2.2 - Caenorhabditis |
| | | | | | | | elegans >gi\|3876766\|emb\|CAA93466.1\| (Z69637) |
| | | | | | | | predicted using Genefinder; Similarity to E. coli |
| | | | | | | | hypothetical protein YCAC (SW: YCAC_ECOLI); |
| | | | | | | | cDNA EST yk555d12.3 comes from this gene |
| | | | | | | | [Caenorhabditis elegans] |
| 1967 | LIB3602-026-Q6-K1-B9 | 7500560 | BLASTX | 214 | 5.00E−17 | 48 | hypothetical protein F35G2.2 - Caenorhabditis |
| | | | | | | | elegans >gi\|3876766\|emb\|CAA93466.1\| (Z69637) |
| | | | | | | | predicted using Genefinder; Similarity to E. coli |
| | | | | | | | hypothetical protein YCAC (SW: YCAC_ECOLI); |
| | | | | | | | cDNA EST yk555d12.3 comes from this gene |
| | | | | | | | [Caenorhabditis elegans] |
| 1968 | LIB3602-063-Q1-K6-H4 | 7486624 | BLASTX | 354 | 3.00E−33 | 46 | hypothetical protein F7N22.3 - Arabidopsis thaliana |
| | | | | | | | >gi\|3047064\|gb\|AAC13578.1\| (AF058825) contains |
| | | | | | | | similarity to peptidyl-prolyl cis-trans isomerase |
| | | | | | | | (Pfam: pro_isomerase.hmm, score: 23.86 and 28.41 |
| | | | | | | | [Arabidopsis thaliana] |
| 1969 | LIB3602-109-Q1-K1-F2 | 7486624 | BLASTX | 329 | 2.00E−30 | 58 | hypothetical protein F7N22.3 - Arabidopsis thaliana |
| | | | | | | | >gi\|3047064\|gb\|AAC13578.1\| (AF058825) contains |
| | | | | | | | similarity to peptidyl-prolyl cis-trans isomerase |
| | | | | | | | (Pfam: pro_isomerase.hmm, score: 23.86 and 28.41 |
| | | | | | | | [Arabidopsis thaliana] |
| 1970 | LIB3602-064-Q1-K6-B6 | 3947719 | BLASTX | 195 | 7.00E−15 | 90 | (AJ012653) ribosomal protein S28 [Prunus persica] |
| | | | | | | | >gi\|3947721\|emb\|CAA10102.1\| (AJ012654) |
| | | | | | | | ribosomal protein S28 [Prunus persica] |
| | | | | | | | >gi\|3947723\|emb\|CAA10103.1\| (AJ012655) |
| | | | | | | | ribosomal protein S28 [Prunus persica] |
| 1971 | LIB3602-063-Q1-K6-H5 | 3947719 | BLASTX | 191 | 2.00E−14 | 97 | (AJ012653) ribosomal protein S28 [Prunus persica] |
| | | | | | | | >gi\|3947721\|emb\|CAA10102.1\| (AJ012654) |
| | | | | | | | ribosomal protein S28 [Prunus persica] |
| | | | | | | | >gi\|3947723\|emb\|CAA10103.1\| (AJ012655) |
| | | | | | | | ribosomal protein S28 [Prunus persica] |
| 1972 | LIB3602-010-Q6-K1-C1 | 5902598 | BLASTX | 484 | 1.00E−48 | 69 | (AF110787) light harvesting complex a protein |
| | | | | | | | [Volvox carteri f. nagariensis] |
| 1973 | LIB3602-077-Q6-K6-B7 | 5902598 | BLASTX | 307 | 1.00E−27 | 48 | (AF110787) light harvesting complex a protein |
| | | | | | | | [Volvox carteri f. nagariensis] |
| 1974 | LIB3602-068-Q1-K1-C1 | 549810 | BLASTX | 575 | 4.00E−59 | 70 | GTP-BINDING PROTEIN YPTV3 |
| | | | | | | | >gi\|486945\|pir\|\|S36366 GTP-binding protein yptV3 - |
| | | | | | | | Volvox carteri >gi\|409164\|gb\|AAA34252.1\| |
| | | | | | | | (L08129) GTP-binding protein [Volvox carteri] |
| 1975 | LIB3602-060-Q6-K1-A10 | 549810 | BLASTX | 402 | 6.00E−39 | 69 | GTP-BINDING PROTEIN YPTV3 |
| | | | | | | | >gi\|486945\|pir\|\|S36366 GTP-binding protein yptV3 - |
| | | | | | | | Volvox carteri >gi\|409164\|gb\|AAA34252.1\| |
| | | | | | | | (L08129) GTP-binding protein [Volvox carteri] |
| 1976 | LIB3602-001-P1-K6-G10 | 4204304 | BLASTX | 629 | 2.00E−65 | 72 | (AC003027) lcl\|prt_seq No definition line found |
| | | | | | | | [Arabidopsis thaliana] |
| 1977 | LIB3602-021-Q6-K1-A6 | 4204304 | BLASTX | 228 | 6.00E−19 | 80 | (AC003027) lcl\|prt_seq No definition line found |
| | | | | | | | [Arabidopsis thaliana] |
| 1978 | LIB3602-089-Q6-K6-A9 | 6934302 | BLASTX | 679 | 2.00E−71 | 98 | (AF221858) polyubiquitin [Euphorbia esula] |
| 1979 | LIB3602-032-Q6-K1-F9 | 6934302 | BLASTX | 327 | 2.00E−30 | 98 | (AF221858) polyubiquitin [Euphorbia esula] |
| 1980 | LIB3602-052-Q6-K1-B5 | 115769 | BLASTX | 301 | 3.00E−27 | 54 | CHLOROPHYLL A-B BINDING PROTEIN OF |
| | | | | | | | LHCII TYPE I (CAB) (LHCP) |
| | | | | | | | >gi\|81190\|pir\|\|S00653 chlorophyll a/b-binding |
| | | | | | | | protein precursor - Euglena gracilis (fragment) |
| | | | | | | | >gi\|829262\|emb\|CAA29821.1\| (X06602) |
| | | | | | | | chlorophyll a/b protein (128 AA) [Euglena gracilis] |
| 1981 | LIB3602-022-Q6-K1-H2 | 115769 | BLASTX | 274 | 5.00E−24 | 55 | CHLOROPHYLL A-B BINDING PROTEIN OF |
| | | | | | | | LHCII TYPE I (CAB) (LHCP) |
| | | | | | | | >gi\|81190\|pir\|\|S00653 chlorophyll a/b-binding |
| | | | | | | | protein precursor - Euglena gracilis (fragment) |
| | | | | | | | >gi\|829262\|emb\|CAA29821.1\| (X06602) |
| | | | | | | | chlorophyll a/b protein (128 AA) [Euglena gracilis] |
| 1982 | LIB3602-070-Q1-K1-A1 | 7108521 | BLASTX | 578 | 2.00E−59 | 65 | (AF127564) ubiquitin-protein ligase 1 [Arabidopsis |
| | | | | | | | thaliana] |
| 1983 | LIB3602-067-Q1-K1-H12 | 7108521 | BLASTX | 520 | 5.00E−55 | 68 | (AF127564) ubiquitin-protein ligase 1 [Arabidopsis |
| | | | | | | | thaliana] |
| 1984 | LIB3602-006-Q1-K1-E6 | 5739404 | BLASTX | 173 | 5.00E−12 | 40 | (AF170026) core subunit of photosystem II |
| | | | | | | | precursor [Chlamydomonas reinhardtii] |
| 1985 | LIB3602-084-Q6-K1-F7 | 5739404 | BLASTX | 156 | 5.00E−10 | 33 | (AF170026) core subunit of photosystem II |
| | | | | | | | precursor [Chlamydomonas reinhardtii] |
| 1986 | LIB3602-030-Q6-K1-A11 | 7486848 | BLASTX | 383 | 8.00E−37 | 60 | hypothetical protein T10P11.1 - Arabidopsis |
| | | | | | | | thaliana >gi\|2262136\|gb\|AAC78251.1\|AAC78251 |
| | | | | | | | (AC002330) predicted protein of unknown function |
| | | | | | | | [Arabidopsis thaliana] |
| | | | | | | | >gi\|4263520\|gb\|AAD15346.1\| (AC004044) |
| | | | | | | | predicted protein of unknown function [Arabidopsis |
| | | | | | | | thalia |
| 1987 | LIB3602-063-Q1-K6-E10 | 7486848 | BLASTX | 375 | 8.00E−36 | 69 | hypothetical protein T10P11.1 - Arabidopsis |
| | | | | | | | thaliana >gi\|2262136\|gb\|AAC78251.1\|AAC78251 |
| | | | | | | | (AC002330) predicted protein of unknown function |
| | | | | | | | [Arabidopsis thaliana] |
| | | | | | | | >gi\|4263520\|gb\|AAD15346.1\| (AC004044) |
| | | | | | | | predicted protein of unknown function [Arabidopsis |
| | | | | | | | thalia |
| 1988 | LIB3602-090-Q6-K6-E11 | 1173218 | BLASTX | 340 | 7.00E−32 | 81 | 40S RIBOSOMAL PROTEIN S15A |
| | | | | | | | >gi\|440824\|gb\|AAA61608.1\| (L27461) ribosomal |
| | | | | | | | protein S15 [Arabidopsis thaliana] |
| | | | | | | | >gi\|2150130\|gb\|AAB58750.1\| (AF001412) |
| | | | | | | | cytoplasmic ribosomal protein S15a [Arabidopsis |
| | | | | | | | thaliana] |
| 1989 | LIB3602-013-Q6-K1-F6 | 1173218 | BLASTX | 284 | 2.00E−25 | 79 | 40S RIBOSOMAL PROTEIN S15A |
| | | | | | | | >gi\|440824\|gb\|AAA61608.1\| (L27461) ribosomal |
| | | | | | | | protein S15 [Arabidopsis thaliana] |
| | | | | | | | >gi\|2150130\|gb\|AAB58750.1\| (AF001412) |
| | | | | | | | cytoplasmic ribosomal protein S15a [Arabidopsis |
| | | | | | | | thaliana] |
| 1990 | LIB3602-070-Q1-K1-G12 | 7489195 | BLASTX | 261 | 2.00E−22 | 47 | ribosomal protein L10, chloroplast - common |
| | | | | | | | tobacco >gi\|3298441\|dbj\|BAA31511.1\| (AB010879) |
| | | | | | | | chloroplast ribosomal protein L10 [Nicotiana |
| | | | | | | | tabacum] |
| 1991 | LIB3602-067-Q1-K1-B1 | 7489195 | BLASTX | 253 | 2.00E−21 | 46 | ribosomal protein L10, chloroplast - common |
| | | | | | | | tobacco >gi\|3298441\|dbj\|BAA31511.1\| (AB010879) |
| | | | | | | | chloroplast ribosomal protein L10 [Nicotiana |
| | | | | | | | tabacum] |
| 1992 | LIB3602-016-Q6-K1-F11 | 7076783 | BLASTX | 438 | 3.00E−43 | 54 | (AL132975) methionyl-tRNA synthetase |
| | | | | | | | (AtcpMetRS) [Arabidopsis thaliana] |
| 1993 | LIB3602-072-Q1-K1-B9 | 7076783 | BLASTX | 162 | 2.00E−14 | 53 | (AL132975) methionyl-tRNA synthetase |
| | | | | | | | (AtcpMetRS) [Arabidopsis thaliana] |
| 1994 | LIB3602-014-Q6-K1-G8 | 586145 | BLASTX | 605 | 1.00E−62 | 69 | UBIQUINOL-CYTOCHROME C REDUCTASE |
| | | | | | | | IRON-SULFUR SUBUNIT, MITOCHONDRIAL |
| | | | | | | | PRECURSOR (RIESKE IRON-SULFUR |
| | | | | | | | PROTEIN) (RISP) >gi\|488848\|emb\|CAA55894.1\| |
| | | | | | | | (X79332) Rieske iron sulphur protein [Solanum |
| | | | | | | | tuberosum] |
| 1995 | LIB3602-072-Q1-K1-F3 | 586145 | BLASTX | 550 | 3.00E−56 | 67 | UBIQUINOL-CYTOCHROME C REDUCTASE |
| | | | | | | | IRON-SULFUR SUBUNIT, MITOCHONDRIAL |
| | | | | | | | PRECURSOR (RIESKE IRON-SULFUR |
| | | | | | | | PROTEIN) (RISP) >gi\|488848\|emb\|CAA55894.1\| |
| | | | | | | | (X79332) Rieske iron sulphur protein [Solanum |
| | | | | | | | tuberosum] |
| 1996 | LIB3602-094-Q6-K6-A6 | 7488696 | BLASTX | 186 | 1.00E−13 | 36 | polyphosphoinositide binding protein Ssh2 - |
| | | | | | | | soybean >gi\|2739046\|gb\|AAB94599.1\| (AF024652) |
| | | | | | | | polyphosphoinositide binding protein Ssh2p |
| | | | | | | | [Glycine max] |
| 1997 | LIB3602-047-Q6-K1-C5 | 7488696 | BLASTX | 162 | 7.00E−11 | 32 | polyphosphoinositide binding protein Ssh2 - |
| | | | | | | | soybean >gi\|2739046\|gb\|AAB94599.1\| (AF024652) |
| | | | | | | | polyphosphoinositide binding protein Ssh2p |
| | | | | | | | [Glycine max] |
| 1998 | LIB3602-114-Q1-K1-B10 | 4185133 | BLASTX | 189 | 7.00E−14 | 45 | (AC005724) putative C3HC4-type RING zinc |
| | | | | | | | finger protein [Arabidopsis thaliana] |
| 1999 | LIB3602-090-Q6-K6-B10 | 4185133 | BLASTX | 185 | 2.00E−13 | 45 | (AC005724) putative C3HC4-type RING zinc |
| | | | | | | | finger protein [Arabidopsis thaliana] |
| 2000 | LIB3602-011-Q6-K1-E6 | 7290666 | BLASTX | 330 | 1.00E−30 | 59 | (AE003436) CG5941 gene product [Drosophila |
| | | | | | | | melanogaster] |
| 2001 | LIB3602-112-Q1-K1-D12 | 7290666 | BLASTX | 147 | 2.00E−09 | 54 | (AE003436) CG5941 gene product [Drosophila |
| | | | | | | | melanogaster] |
| 2002 | LIB3602-069-Q1-K1-G4 | 7291506 | BLASTX | 192 | 3.00E−14 | 42 | (AE003459) CG3800 gene product [Drosophila |
| | | | | | | | melanogaster] |
| 2003 | LIB3602-086-Q6-K1-D2 | 7291506 | BLASTX | 192 | 3.00E−14 | 42 | (AE003459) CG3800 gene product [Drosophila |
| | | | | | | | melanogaster] |
| 2004 | LIB3602-104-Q1-K1-F5 | 266685 | BLASTX | 163 | 7.00E−11 | 72 | DIHYDROLIPOAMIDE |
| | | | | | | | ACETYLTRANSFERASE COMPONENT OF |
| | | | | | | | PYRUVATE DEHYDROGENASE COMPLEX |
| | | | | | | | (E2) (PDC-E2) (70 KD MITOCHONDRIAL |
| | | | | | | | AUTOANTIGEN OF PRIMARY BILIARY |
| | | | | | | | CIRRHOSIS) (PBC) >gi\|111580\|pir\|\|S21766 |
| | | | | | | | dihydrolipoamide S-acetyltransferase (EC 2.3.1.12) - |
| | | | | | | | rat (fragment) |
| 2005 | LIB3602-104-Q1-K1-H6 | 266685 | BLASTX | 163 | 7.00E−11 | 72 | DIHYDROLIPOAMIDE |
| | | | | | | | ACETYLTRANSFERASE COMPONENT OF |
| | | | | | | | PYRUVATE DEHYDROGENASE COMPLEX |
| | | | | | | | (E2) (PDC-E2) (70 KD MITOCHONDRIAL |
| | | | | | | | AUTOANTIGEN OF PRIMARY BILIARY |
| | | | | | | | CIRRHOSIS) (PBC) >gi\|111580\|pir\|\|S21766 |
| | | | | | | | dihydrolipoamide S-acetyltransferase (EC 2.3.1.12) - |
| | | | | | | | rat (fragment) |
| 2006 | LIB3602-012-Q6-K1-G1 | 3015514 | BLASTX | 533 | 3.00E−54 | 55 | (U72351) ADPG pyrophosphorylase small subunit |
| | | | | | | | [Arabidopsis thaliana] |
| | | | | | | | >gi\|7242894\|dbj\|BAA92523.1\| (AB039889) |
| | | | | | | | glucose-1-phosphate adenylyltransferase |
| | | | | | | | [Arabidopsis thaliana] |
| 2007 | LIB3602-066-Q1-K6-C1 | 3015514 | BLASTX | 522 | 6.00E−53 | 55 | (U72351) ADPG pyrophosphorylase small subunit |
| | | | | | | | [Arabidopsis thaliana] |
| | | | | | | | >gi\|7242894\|dbj\|BAA92523.1\| (AB039889) |
| | | | | | | | glucose-1-phosphate adenylyltransferase |
| | | | | | | | [Arabidopsis thaliana] |
| 2008 | LIB3602-107-Q1-K1-A10 | 118024 | BLASTX | 209 | 2.00E−16 | 72 | CYTOCHROME C >gi\|65518\|pir\|\|CCSP |
| | | | | | | | cytochrome c - spinach |
| 2009 | LIB3602-120-Q1-K1-G8 | 118024 | BLASTX | 204 | 4.00E−16 | 82 | CYTOCHROME C >gi\|65518\|pir\|\|CCSP |
| | | | | | | | cytochrome c - spinach |
| 2010 | LIB3602-022-Q6-K1-D5 | 7299915 | BLASTN | 45 | 5.00E−16 | 89 | Drosophila melanogaster genomic scaffold |
| | | | | | | | 142000013386035 section 31 of 105, complete |
| | | | | | | | sequence |
| 2011 | LIB3602-068-Q1-K1-F7 | 7299915 | BLASTN | 43 | 2.00E−14 | 88 | Drosophila melanogaster genomic scaffold |
| | | | | | | | 142000013386035 section 31 of 105, complete |
| | | | | | | | sequence |
| 2012 | LIB3602-083-Q6-K6-A3 | 7629994 | BLASTX | 278 | 8.00E−25 | 73 | (AL132960) 60S RIBOSOMAL PROTEIN L36 |
| | | | | | | | homolog [Arabidopsis thaliana] |
| 2013 | LIB3602-035-Q1-K1-D7 | 7629994 | BLASTX | 252 | 1.00E−21 | 71 | (AL132960) 60S RIBOSOMAL PROTEIN L36 |
| | | | | | | | homolog [Arabidopsis thaliana] |
| 2014 | LIB3602-039-Q6-K1-B10 | 7498202 | BLASTX | 327 | 5.00E−30 | 52 | hypothetical protein D2030.5 - Caenorhabditis |
| | | | | | | | elegans >gi\|3875398\|emb\|CAA98118.1\| (Z73906) |
| | | | | | | | Similarity to B. subtilis YQJC protein |
| | | | | | | | (TR: G1303954); cDNA EST EMBL: T01187 comes |
| | | | | | | | from this gene [Caenorhabditis elegans] |
| 2015 | LIB3602-004-Q1-K1-B8 | 7498202 | BLASTX | 322 | 2.00E−29 | 51 | hypothetical protein D2030.5 - Caenorhabditis |
| | | | | | | | elegans >gi\|3875398\|emb\|CAA98118.1\| (Z73906) |
| | | | | | | | Similarity to B. subtilis YQJC protein |
| | | | | | | | (TR: G1303954); cDNA EST EMBL: T01187 comes |
| | | | | | | | from this gene [Caenorhabditis elegans] |
| 2016 | LIB3602-052-Q6-K1-G1 | 7492314 | BLASTX | 303 | 2.00E−27 | 51 | pop-interacting protein 1 - fission yeast |
| | | | | | | | (Schizosaccharomyces pombe) |
| | | | | | | | >gi\|5853264\|gb\|AAD54393.1\|AF179228_1 |
| | | | | | | | (AF179228) pop-interacting protein 1 |
| | | | | | | | [Schizosaccharomyces pombe] |
| | | | | | | | >gi\|6073753\|emb\|CAB58559.1\| (Z98977) pop- |
| | | | | | | | interacting protein 1. [Schizosaccharomyce |
| 2017 | LIB3602-115-Q1-K1-B11 | 7492314 | BLASTX | 302 | 3.00E−27 | 51 | pop-interacting protein 1 - fission yeast |
| | | | | | | | (Schizosaccharomyces pombe) |
| | | | | | | | >gi\|5853264\|gb\|AAD54393.1\|AF179228_1 |
| | | | | | | | (AF179228) pop-interacting protein 1 |
| | | | | | | | [Schizosaccharomyces pombe] |
| | | | | | | | >gi\|6073753\|emb\|CAB58559.1\| (Z98977) pop- |
| | | | | | | | interacting protein 1. [Schizosaccharomyce |
| 2018 | LIB3602-022-Q6-K1-C5 | 6015158 | BLASTX | 364 | 1.00E−34 | 62 | FK506-BINDING PROTEIN (FKBP) (PEPTIDYL- |
| | | | | | | | PROLYL CIS-TRANS ISOMERASE) (PPIASE) |
| | | | | | | | >gi\|7492278\|pir\|\|T40724 peptidyl-prolyl cis-trans |
| | | | | | | | isomerase - fission yeast (Schizosaccharomyces |
| | | | | | | | pombe) >gi\|2879879\|emb\|CAA17020.1\| |
| | | | | | | | (AL021816) peptidyl-prolyl cis-trans isomerase; |
| 2019 | LIB3602-101-Q1-K1-F5 | 6015158 | BLASTX | 253 | 8.00E−28 | 67 | FK506-BINDING PROTEIN (FKBP) (PEPTIDYL- |
| | | | | | | | PROLYL CIS-TRANS ISOMERASE) (PPIASE) |
| | | | | | | | >gi\|7492278\|pir\|\|T40724 peptidyl-prolyl cis-trans |
| | | | | | | | isomerase - fission yeast (Schizosaccharomyces |
| | | | | | | | pombe) >gi\|2879879\|emb\|CAA17020.1\| |
| | | | | | | | (AL021816) peptidyl-prolyl cis-trans isomerase; |
| 2020 | LIB3602-107-Q1-K1-A12 | 2500365 | BLASTX | 176 | 2.00E−12 | 36 | PROBABLE 60 RIBOSOMAL PROTEIN L14 |
| | | | | | | | (HYDROXYPROLINE RICH GLYCOPROTEIN |
| | | | | | | | HRGP1) |
| 2021 | LIB3602-006-Q1-K1-E4 | 2500365 | BLASTX | 171 | 6.00E−12 | 38 | PROBABLE 60 RIBOSOMAL PROTEIN L14 |
| | | | | | | | (HYDROXYPROLINE RICH GLYCOPROTEIN |
| | | | | | | | HRGP1) |
| 2022 | LIB3602-067-Q1-K1-F3 | 7485991 | BLASTX | 282 | 8.00E−25 | 44 | hypothetical protein F22O13.29 - Arabidopsis |
| | | | | | | | thaliana >gi\|3063467\|gb\|AAC14055.1\| (AC003981) |
| | | | | | | | F22O13.29 [Arabidopsis thaliana] |
| | | | | | | | >gi\|6996317\|emb\|CAB75510.1\| (AJ251088) ABI3- |
| | | | | | | | interacting protein 3, AIP3 [Arabidopsis thaliana] |
| 2023 | LIB3602-084-Q6-K1-H7 | 7485991 | BLASTX | 177 | 2.00E−16 | 45 | hypothetical protein F22O13.29 - Arabidopsis |
| | | | | | | | thaliana >gi\|3063467\|gb\|AAC14055.1\| (AC003981) |
| | | | | | | | F22O13.29 [Arabidopsis thaliana] |
| | | | | | | | >gi\|6996317\|emb\|CAB75510.1\| (AJ251088) ABI3- |
| | | | | | | | interacting protein 3, AIP3 [Arabidopsis thaliana] |
| 2024 | LIB3602-001-P1-K6-H8 | 585338 | BLASTX | 755 | 3.00E−80 | 72 | ADENYLATE KINASE B (ATP-AMP |
| | | | | | | | TRANSPHOSPHORYLASE) |
| | | | | | | | >gi\|391879\|dbj\|BAA01181.1\| (D10335) adenylate |
| | | | | | | | kinase-b [Oryza sativa] |
| 2025 | LIB3602-037-Q6-K1-D7 | 585338 | BLASTX | 376 | 5.00E−36 | 64 | ADENYLATE KINASE B (ATP-AMP |
| | | | | | | | TRANSPHOSPHORYLASE) |
| | | | | | | | >gi\|391879\|dbj\|BAA01181.1\| (D10335) adenylate |
| | | | | | | | kinase-b [Oryza sativa] |
| 2026 | LIB3602-118-Q1-K1-D12 | 7488897 | BLASTX | 292 | 5.00E−26 | 57 | hypothetical protein - castor bean |
| | | | | | | | >gi\|1632831\|emb\|CAA89698.1\| (Z49698) orf |
| | | | | | | | [Ricinus communis] |
| 2027 | LIB3602-084-Q6-K1-A2 | 7488897 | BLASTX | 196 | 6.00E−15 | 71 | hypothetical protein - castor bean |
| | | | | | | | >gi\|1632831\|emb\|CAA89698.1\| (Z49698) orf |
| | | | | | | | [Ricinus communis] |
| 2028 | LIB3602-045-Q6-K1-F2 | 7362767 | BLASTX | 402 | 5.00E−39 | 54 | (AL162651) 60S RIBOSOMAL PROTEIN L7A |
| | | | | | | | protein [Arabidopsis thaliana] |
| 2029 | LIB3602-012-Q6-K1-B12 | 7362767 | BLASTX | 152 | 9.00E−10 | 57 | (AL162651) 60S RIBOSOMAL PROTEIN L7A |
| | | | | | | | protein [Arabidopsis thaliana] |
| 2030 | LIB3602-114-Q1-K1-B1 | 6006684 | BLASTX | 247 | 9.00E−21 | 44 | (AF089106) unknown [Homo sapiens] |
| 2031 | LIB3602-088-Q6-K6-D4 | 6006684 | BLASTX | 236 | 2.00E−19 | 44 | (AF089106) unknown [Homo sapiens] |
| 2032 | LIB3602-100-Q1-K1-E11 | 2398681 | BLASTX | 390 | 1.00E−37 | 61 | (Y14798) 3-deoxy-D-arabino-heptulosonate 7- |
| | | | | | | | phosphate synthase [Morinda citrifolia] |
| 2033 | LIB3602-066-Q1-K6-E6 | 2398681 | BLASTX | 370 | 4.00E−35 | 58 | (Y14798) 3-deoxy-D-arabino-heptulosonate 7- |
| | | | | | | | phosphate synthase [Morinda citrifolia] |
| 2034 | LIB3602-034-Q6-K1-F11 | 6714302 | BLASTX | 189 | 4.00E−14 | 67 | (AC013354) F15H18.8 [Arabidopsis thaliana] |
| 2035 | LIB3602-035-Q1-K1-F11 | 6714302 | BLASTX | 171 | 5.00E−12 | 68 | (AC013354) F15H18.8 [Arabidopsis thaliana] |
| 2036 | LIB3602-068-Q1-K1-E6 | 6687300 | BLASTN | 38 | 8.00E−12 | 92 | Cyanophora paradoxa partial mRNA for 60S |
| | | | | | | | ribosomal protein L7 |
| 2037 | LIB3602-036-Q6-K1-D10 | 6687300 | BLASTN | 33 | 7.00E−09 | 93 | Cyanophora paradoxa partial mRNA for 60S |
| | | | | | | | ribosomal protein L7 |
| 2038 | LIB3602-106-Q1-K1-G2 | 3747049 | BLASTN | 86 | 3.00E−40 | 87 | Zea mays ribosomal protein L26 mRNA, partial cds |
| 2039 | LIB3602-005-Q1-K1-F8 | 3747049 | BLASTN | 61 | 3.00E−25 | 89 | Zea mays ribosomal protein L26 mRNA, partial cds |
| 2040 | LIB3602-047-Q6-K1-G12 | 4507873 | BLASTX | 280 | 1.00E−24 | 40 | von Hippel-Lindau binding protein 1 |
| | | | | | | | >gi\|3212112\|emb\|CAA76761.1\| (Y17394) prefoldin |
| | | | | | | | subunit 3 [Homo sapiens] |
| 2041 | LIB3602-030-Q6-K1-H6 | 4507873 | BLASTX | 251 | 3.00E−21 | 39 | von Hippel-Lindau binding protein 1 |
| | | | | | | | >gi\|3212112\|emb\|CAA76761.1\| (Y17394) prefoldin |
| | | | | | | | subunit 3 [Homo sapiens] |
| 2042 | LIB3602-069-Q1-K1-G7 | 3860277 | BLASTX | 579 | 1.00E−59 | 62 | (AC005824) 60S ribosomal protein L10A |
| | | | | | | | [Arabidopsis thaliana] |
| 2043 | LIB3602-025-Q6-K1-G3 | 3860277 | BLASTX | 415 | 1.00E−40 | 70 | (AC005824) 60S ribosomal protein L10A |
| | | | | | | | [Arabidopsis thaliana] |
| 2044 | LIB3602-011-Q6-K1-C8 | 2499932 | BLASTX | 298 | 9.00E−27 | 59 | ADENINE PHOSPHORIBOSYLTRANSFERASE |
| | | | | | | | 1 (APRT) >gi\|7433931\|pir\|\|T06263 adenine |
| | | | | | | | phosphoribosyltransferase (EC 2.4.2.7) APT1 - |
| | | | | | | | wheat >gi\|726305\|gb\|AAA80609.1\| (U22442) |
| | | | | | | | adenine phosphoribosyltransferase form 1 [Triticum |
| | | | | | | | aestivum] |
| 2045 | LIB3602-084-Q6-K1-G3 | 2499932 | BLASTX | 166 | 9.00E−20 | 66 | ADENINE PHOSPHORIBOSYLTRANSFERASE |
| | | | | | | | 1 (APRT) >gi\|7433931\|pir\|\|T06263 adenine |
| | | | | | | | phosphoribosyltransferase (EC 2.4.2.7) APT1 - |
| | | | | | | | wheat >gi\|726305\|gb\|AAA80609.1\| (U22442) |
| | | | | | | | adenine phosphoribosyltransferase form 1 [Triticum |
| | | | | | | | aestivum] |
| 2046 | LIB3602-114-Q1-K1-F11 | 7487365 | BLASTX | 408 | 1.00E−39 | 57 | hypothetical protein T23J7.140 - Arabidopsis |
| | | | | | | | thaliana >gi\|4741198\|emb\|CAB41864.1\| |
| | | | | | | | (AL049746) putative protein [Arabidopsis thaliana] |
| 2047 | LIB3602-118-Q1-K1-G2 | 7487365 | BLASTX | 286 | 1.00E−35 | 60 | hypothetical protein T23J7.140 - Arabidopsis |
| | | | | | | | thaliana >gi\|4741198\|emb\|CAB41864.1\| |
| | | | | | | | (AL049746) putative protein [Arabidopsis thaliana] |
| 2048 | LIB3602-013-Q6-K1-B4 | 3387899 | BLASTX | 789 | 2.00E−84 | 88 | (AF070540) putative nuclear protein [Homo |
| | | | | | | | sapiens] |
| 2049 | LIB3602-087-Q6-K1-G7 | 3387899 | BLASTX | 597 | 9.00E−62 | 67 | (AF070540) putative nuclear protein [Homo |
| | | | | | | | sapiens] |
| 2050 | LIB3602-010-Q6-K1-F11 | 464621 | BLASTX | 465 | 2.00E−46 | 64 | 60S RIBOSOMAL PROTEIN L6 (YL16-LIKE) |
| | | | | | | | >gi\|280374\|pir\|\|S28586 ribosomal protein ML16, |
| | | | | | | | cytosolic - common ice plant |
| | | | | | | | >gi\|19539\|emb\|CAA49175.1\| (X69378) ribosomal |
| | | | | | | | protein YL16 [Mesembryanthemum crystallinum] |
| 2051 | LIB3602-039-Q6-K1-G8 | 464621 | BLASTX | 433 | 1.00E−42 | 65 | 60S RIBOSOMAL PROTEIN L6 (YL16-LIKE) |
| | | | | | | | >gi\|280374\|pir\|\|S28586 ribosomal protein ML16, |
| | | | | | | | cytosolic - common ice plant |
| | | | | | | | >gi\|19539\|emb\|CAA49175.1\| (X69378) ribosomal |
| | | | | | | | protein YL16 [Mesembryanthemum crystallinum] |
| 2052 | LIB3602-058-Q6-K1-E4 | 6446570 | BLASTX | 449 | 2.00E−44 | 71 | (AU066541) hypothetical protein [Chlamydomonas |
| | | | | | | | sp.] |
| 2053 | LIB3602-046-Q6-K1-G7 | 6446570 | BLASTX | 446 | 3.00E−44 | 70 | (AU066541) hypothetical protein [Chlamydomonas |
| | | | | | | | sp.] |
| 2054 | LIB3602-042-Q6-K1-E3 | 7445817 | BLASTX | 356 | 2.00E−33 | 59 | ABC-type transport protein slr0864 - Synechocystis |
| | | | | | | | sp. (strain PCC 6803) |
| | | | | | | | >gi\|1652892\|dbj\|BAA17810.1\| (D90909) ABC |
| | | | | | | | transporter [Synechocystis sp.] |
| 2055 | LIB3602-003-Q1-K1-C4 | 7445817 | BLASTX | 332 | 9.00E−31 | 61 | ABC-type transport protein slr0864 - Synechocystis |
| | | | | | | | sp. (strain PCC 6803) |
| | | | | | | | >gi\|1652892\|dbj\|BAA17810.1\| (D90909) ABC |
| | | | | | | | transporter [Synechocystis sp.] |
| 2056 | LIB3602-118-Q1-K1-E9 | 18163 | BLASTN | 39 | 3.00E−12 | 85 | C. reinhardtii psb3 mRNA for OEE3 protein of |
| | | | | | | | photosystem II (oxygen-evolving protein) |
| 2057 | LIB3602-035-Q1-K1-E4 | 18163 | BLASTN | 37 | 6.00E−11 | 97 | C. reinhardtii psb3 mRNA for OEE3 protein of |
| | | | | | | | photosystem II (oxygen-evolving protein) |
| 2058 | LIB3602-065-Q1-K6-F12 | 1361925 | BLASTX | 283 | 6.00E−25 | 36 | translation elongation factor aEF-1 alpha chain- |
| | | | | | | | Desulfurococcus mobilis |
| 2059 | LIB3602-071-Q1-K1-B5 | 1361925 | BLASTX | 257 | 7.00E−22 | 37 | translation elongation factor aEF-1 alpha chain - |
| | | | | | | | Desulfurococcus mobilis |
| 2060 | LIB3602-026-Q6-K1-C9 | 5442410 | BLASTX | 304 | 1.00E−32 | 52 | (AF159254) ascorbate peroxidase [Zantedeschia |
| | | | | | | | aethiopica] |
| 2061 | LIB3602-005-Q1-K1-G1 | 5442410 | BLASTX | 298 | 9.00E−27 | 41 | (AF159254) ascorbate peroxidase [Zantedeschia |
| | | | | | | | aethiopica] |
| 2062 | LIB3602-029-Q6-K1-B12 | 2739219 | BLASTX | 248 | 5.00E−21 | 92 | (AJ001161) rpS28 [Hordeum vulgare] |
| 2063 | LIB3602-030-Q6-K1-F5 | 2739219 | BLASTX | 248 | 5.00E−21 | 92 | (AJ001161) rpS28 [Hordeum vulgare] |
| 2064 | LIB3602-090-Q6-K6-C8 | 6633821 | BLASTX | 180 | 7.00E−13 | 39 | (AC009519) F1N19.23 [Arabidopsis thaliana] |
| 2065 | LIB3602-091-Q6-K6-C8 | 6633821 | BLASTX | 180 | 7.00E−13 | 39 | (AC009519) F1N19.23 [Arabidopsis thaliana] |
| 2066 | LIB3602-001-P1-K6-B1 | 6320203 | BLASTX | 262 | 2.00E−22 | 38 | Ydl001wp >gi\|1077511\|pir\|\|S50981 probable |
| | | | | | | | membrane protein YDL001w - yeast |
| | | | | | | | (Saccharomyces cerevisiae) |
| | | | | | | | >gi\|642805\|emb\|CAA88060.1\| (Z48008) unknown |
| | | | | | | | [Saccharomyces cerevisiae] |
| 2067 | LIB3602-002-P1-K6-C4 | 6320203 | BLASTX | 194 | 1.00E−14 | 34 | Ydl001wp >gi\|1077511\|pir\|\|S50981 probable |
| | | | | | | | membrane protein YDL001w - yeast |
| | | | | | | | (Saccharomyces cerevisiae) |
| | | | | | | | >gi\|642805\|emb\|CAA88060.1\| (Z48008) unknown |
| | | | | | | | [Saccharomyces cerevisiae] |
| 2068 | LIB3602-065-Q1-K6-D12 | 7488076 | BLASTX | 532 | 3.00E−54 | 67 | probable clathrin-associated protein F23E12.30 - |
| | | | | | | | Arabidopsis thaliana >gi\|2231702\|gb\|AAB96889.1\| |
| | | | | | | | (U92086) clathrin assembly protein AP19 homolog |
| | | | | | | | [Arabidopsis thaliana] |
| | | | | | | | >gi\|3080409\|emb\|CAA18728.1\| (AL022604) |
| | | | | | | | clathrin assembly protein AP19 homolog [Arabidop |
| 2069 | LIB3602-004-Q1-K1-H1 | 7488076 | BLASTX | 190 | 4.00E−14 | 64 | probable clathrin-associated protein F23E12.30 - |
| | | | | | | | Arabidopsis thaliana >gi\|2231702\|gb\|AAB96889.1\| |
| | | | | | | | (U92086) clathrin assembly protein AP19 homolog |
| | | | | | | | [Arabidopsis thaliana] |
| | | | | | | | >gi\|3080409\|emb\|CAA18728.1\| (AL022604) |
| | | | | | | | clathrin assembly protein AP19 homolog [Arabidop |
| 2070 | LIB3602-070-Q1-K1-F10 | 6321354 | BLASTX | 430 | 4.00E−42 | 43 | Ygl084cp >gi\|1723878\|sp\|P53154\|YGI4_YEAST |
| | | | | | | | HYPOTHETICAL 65.3 KD PROTEIN IN MAD1- |
| | | | | | | | SCY1 INTERGENIC REGION |
| | | | | | | | >gi\|2132541\|pir\|\|S64091 probable membrane |
| | | | | | | | protein YGL084c - yeast (Saccharomyces |
| | | | | | | | cerevisiae) >gi\|1322607\|emb\|CAA96789.1\| |
| | | | | | | | (Z72606) ORF YGL084c [Saccharomy |
| 2071 | LIB3602-054-Q6-K1-E2 | 6321354 | BLASTX | 209 | 2.00E−16 | 35 | Ygl084cp >gi\|1723878\|sp\|P53154\|YGI4_YEAST |
| | | | | | | | HYPOTHETICAL 65.3 KD PROTEIN IN MAD1- |
| | | | | | | | SCY1 INTERGENIC REGION |
| | | | | | | | >gi\|2132541\|pir\|\|S64091 probable membrane |
| | | | | | | | protein YGL084c - yeast (Saccharomyces |
| | | | | | | | cerevisiae) >gi\|1322607\|emb\|CAA96789.1\| |
| | | | | | | | (Z72606) ORF YGL084c [Saccharomy |
| 2072 | LIB3602-062-Q6-K1-H5 | 1707012 | BLASTX | 692 | 6.00E−73 | 72 | (U78721) putative tyrosyl-tRNA synthetase |
| | | | | | | | [Arabidopsis thaliana] |
| 2073 | LIB3602-062-Q6-K1-H6 | 1707012 | BLASTX | 301 | 5.00E−27 | 64 | (U78721) putative tyrosyl-tRNA synthetase |
| | | | | | | | [Arabidopsis thaliana] |
| 2074 | LIB3602-107-Q1-K1-H1 | 7543908 | BLASTX | 600 | 4.00E−62 | 66 | (AL163572) putative protein [Arabidopsis thaliana] |
| 2075 | LIB3602-079-Q6-K6-H1 | 7543908 | BLASTX | 236 | 2.00E−19 | 75 | (AL163572) putative protein [Arabidopsis thaliana] |
| 2076 | LIB3602-089-Q6-K6-E6 | 1170507 | BLASTX | 550 | 3.00E−56 | 92 | EUKARYOTIC INITIATION FACTOR 4A-3 (EIF- |
| | | | | | | | 4A-3) >gi\|100276\|pir\|\|S22579 translation initiation |
| | | | | | | | factor eIF-4A - curled-leaved tobacco |
| | | | | | | | >gi\|19699\|emb\|CAA43514.1\| (X61206) nicotiana |
| | | | | | | | eukaryotic translation initiation factor 4A |
| | | | | | | | [Nicotiana plumbaginifolia] |
| 2077 | LIB3602-118-Q1-K1-H8 | 1170507 | BLASTX | 403 | 7.00E−55 | 95 | EUKARYOTIC INITIATION FACTOR 4A-3 (EIF- |
| | | | | | | | 4A-3) >gi\|100276\|pir\|\|S22579 translation initiation |
| | | | | | | | factor eIF-4A - curled-leaved tobacco |
| | | | | | | | >gi\|19699\|emb\|CAA43514.1\| (X61206) nicotiana |
| | | | | | | | eukaryotic translation initiation factor 4A |
| | | | | | | | [Nicotiana plumbaginifolia] |
| 2078 | LIB3602-010-Q6-K1-G3 | 7378766 | BLASTX | 704 | 2.00E−74 | 75 | (AJ277097) putative kinetochore protein [Hordeum |
| | | | | | | | vulgare] |
| 2079 | LIB3602-100-Q1-K1-B6 | 7378766 | BLASTX | 575 | 3.00E−59 | 72 | (AJ277097) putative kinetochore protein [Hordeum |
| | | | | | | | vulgare] |
| 2080 | LIB3602-100-Q1-K1-C6 | 3881976 | BLASTX | 173 | 3.00E−24 | 74 | (AJ012409) hypothetical protein [Homo sapiens] |
| 2081 | LIB3602-108-Q1-K1-C6 | 3881976 | BLASTX | 162 | 4.00E−23 | 70 | (AJ012409) hypothetical protein [Homo sapiens] |
| 2082 | LIB3602-065-Q1-K6-D11 | 5817608 | BLASTX | 349 | 8.00E−33 | 46 | (AF137288) putative translation initiation factor 2B |
| | | | | | | | beta subunit [Nicotiana tabacum] |
| 2083 | LIB3602-111-Q1-K1-H6 | 5817608 | BLASTX | 197 | 2.00E−15 | 45 | (AF137288) putative translation initiation factor 2B |
| | | | | | | | beta subunit [Nicotiana tabacum] |
| 2084 | LIB3602-100-Q1-K1-C8 | 7431833 | BLASTX | 308 | 7.00E−44 | 67 | NAD(P)+ transhydrogenase (B-specific) (EC |
| | | | | | | | 1.6.1.1) precursor, mitochondrial - human |
| | | | | | | | >gi\|1110520\|gb\|AAC51914.1\| (U40490) |
| | | | | | | | nicotinamide nucleotide transhydrogenase [Homo |
| | | | | | | | sapiens] |
| 2085 | LIB3602-001-P1-K6-A11 | 7431833 | BLASTX | 245 | 2.00E−20 | 67 | NAD(P)+ transhydrogenase (B-specific) (EC |
| | | | | | | | 1.6.1.1) precursor, mitochondrial - human |
| | | | | | | | >gi\|1110520\|gb\|AAC51914.1\| (U40490) |
| | | | | | | | nicotinamide nucleotide transhydrogenase [Homo |
| | | | | | | | sapiens] |
| 2086 | LIB3602-067-Q1-K1-G4 | 465847 | BLASTX | 697 | 2.00E−73 | 67 | HYPOTHETICAL 66.5 KD PROTEIN F02A9.5 IN |
| | | | | | | | CHROMOSOME III >gi\|280542\|pir\|\|S28313 |
| | | | | | | | hypothetical protein F02A9.5 - Caenorhabditis |
| | | | | | | | elegans >gi\|3875448\|emb\|CAA79618.1\| (Z19555) |
| | | | | | | | predicted using Genefinder; similar to Propionyl- |
| | | | | | | | CoA carboxylase beta chain; cDNA EST |
| 2087 | LIB3602-104-Q1-K1-F2 | 465847 | BLASTX | 141 | 4.00E−09 | 66 | HYPOTHETICAL 66.5 KD PROTEIN F02A9.5 IN |
| | | | | | | | CHROMOSOME III >gi\|280542\|pir\|\|S28313 |
| | | | | | | | hypothetical protein F02A9.5 - Caenorhabditis |
| | | | | | | | elegans >gi\|3875448\|emb\|CAA79618.1\| (Z19555) |
| | | | | | | | predicted using Genefinder; similar to Propionyl- |
| | | | | | | | CoA carboxylase beta chain; cDNA EST |
| 2088 | LIB3602-086-Q6-K1-F2 | 7469487 | BLASTX | 178 | 1.00E−12 | 37 | hypothetical protein - Synechocystis sp. (strain PCC |
| | | | | | | | 6803) >gi\|1653572\|dbj\|BAA18485.1\| (D90914) |
| | | | | | | | sterol-C-methyltransferase [Synechocystis sp.] |
| 2089 | LIB3602-067-Q1-K1-E12 | 7469487 | BLASTX | 177 | 2.00E−12 | 42 | hypothetical protein - Synechocystis sp. (strain PCC |
| | | | | | | | 6803) >gi\|1653572\|dbj\|BAA18485.1\| (D90914) |
| | | | | | | | sterol-C-methyltransferase [Synechocystis sp.] |
| 2090 | LIB3602-022-Q6-K1-F3 | 266947 | BLASTX | 225 | 3.00E−18 | 47 | 60S ACIDIC RIBOSOMAL PROTEIN P1 |
| | | | | | | | >gi\|71168\|- Chlamydomonas reinhardtii |
| | | | | | | | >gi\|18211\|emb\|CAA47042.1\| (X66411) ribosomal |
| | | | | | | | protein P1 [Chlamydomonas reinhardtii] |
| 2091 | LIB3602-042-Q6-K1-F8 | 266947 | BLASTX | 148 | 3.00E−09 | 40 | 60S ACIDIC RIBOSOMAL PROTEIN P1 |
| | | | | | | | >gi\|71168\|pir\|\|R6KM1C acidic ribosomal protein |
| | | | | | | | P1, cytosolic - Chlamydomonas reinhardtii |
| | | | | | | | >gi\|18211\|emb\|CAA47042.1\| (X66411) ribosomal |
| | | | | | | | protein P1 [Chlamydomonas reinhardtii] |
| 2092 | LIB3602-069-Q1-K1-E7 | 4218951 | BLASTX | 399 | 2.00E−38 | 80 | (AF081796) fructose-1,6-bisphosphatase precursor |
| | | | | | | | [Brassica napus] |
| 2093 | LIB3602-086-Q6-K1-F3 | 4218951 | BLASTX | 210 | 2.00E−16 | 65 | (AF081796) fructose-1,6-bisphosphatase precursor |
| | | | | | | | [Brassica napus] |
| 2094 | LIB3602-041-Q6-K1-C4 | 6815060 | BLASTX | 894 | 1.00E−96 | 96 | (AP001080) EST C26994(C50622) corresponds to |
| | | | | | | | a region of the predicted gene.; Similar to ADP- |
| | | | | | | | ribosylation factor. (D17760) [Oryza sativa] |
| | | | | | | | >gi\|7242921\|dbj\|BAA92519.1\| (AP001383) EST |
| | | | | | | | C26994(C50622) corresponds to a region of the |
| | | | | | | | predicted gene.; Similar to |
| 2095 | LIB3602-058-Q6-K1-B8 | 6815060 | BLASTX | 634 | 3.00E−66 | 97 | (AP001080) EST C26994(C50622) corresponds to |
| | | | | | | | a region of the predicted gene.; Similar to ADP- |
| | | | | | | | ribosylation factor. (D17760) [Oryza sativa] |
| | | | | | | | >gi\|7242921\|dbj\|BAA92519.1\| (AP001383) EST |
| | | | | | | | C26994(C50622) corresponds to a region of the |
| | | | | | | | predicted gene.; Similar to |
| 2096 | LIB3602-060-Q6-K1-G3 | 4138855 | BLASTX | 489 | 4.00E−49 | 54 | (AF098072) IMMUTANS [Arabidopsis thaliana] |
| 2097 | LIB3602-017-Q6-K1-C2 | 4138855 | BLASTX | 475 | 2.00E−47 | 53 | (AF098072) IMMUTANS [Arabidopsis thaliana] |
| 2098 | LIB3602-112-Q1-K1-A4 | 4759280 | BLASTX | 194 | 1.00E−14 | 68 | U5 snRNP-specific protein, 116 kD |
| | | | | | | | >gi\|434759\|dbj\|BAA04699.1\| (D21163) similar to |
| | | | | | | | human elongation factor 2 mRNA (HSEF2). [Homo |
| | | | | | | | sapiens] |
| 2099 | LIB3602-089-Q6-K6-A4 | 4759280 | BLASTX | 190 | 5.00E−14 | 66 | U5 snRNP-specific protein, 116 kD |
| | | | | | | | >gi\|434759\|dbj\|BAA04699.1\| (D21163) similar to |
| | | | | | | | human elongation factor 2 mRNA (HSEF2). [Homo |
| | | | | | | | sapiens] |
| 2100 | LIB3602-074-Q1-K1-E1 | 3687251 | BLASTX | 183 | 2.00E−13 | 42 | (AC005169) putative ribosomal protein L28 |
| | | | | | | | [Arabidopsis thaliana] |
| 2101 | LIB3602-087-Q6-K1-A2 | 3687251 | BLASTX | 177 | 1.00E−12 | 40 | (AC005169) putative ribosomal protein L28 |
| | | | | | | | [Arabidopsis thaliana] |
| 2102 | LIB3602-050-Q6-K1-C2 | 7489246 | BLASTX | 374 | 9.00E−36 | 55 | oxoglutarate/malate translocator - potato |
| | | | | | | | >gi\|1486472\|emb\|CAA68164.1\| (X99853) |
| | | | | | | | oxoglutarate malate translocator [Solanum |
| | | | | | | | tuberosum] |
| 2103 | LIB3602-063-Q1-K6-D5 | 7489246 | BLASTX | 357 | 1.00E−33 | 49 | oxoglutarate/malate translocator - potato |
| | | | | | | | >gi\|1486472\|emb\|CAA68164.1\| (X99853) |
| | | | | | | | oxoglutarate malate translocator [Solanum |
| | | | | | | | tuberosum] |
| 2104 | LIB3602-012-Q6-K1-A12 | 7435806 | BLASTX | 356 | 2.00E−33 | 48 | cysteine proteinase mir3 (EC 3.4.22.—) - maize |
| | | | | | | | >gi\|2425066\|gb\|AAB88263.1\| (AF019147) cysteine |
| | | | | | | | proteinase Mir3 [Zea mays] |
| 2105 | LIB3602-036-Q6-K1-F11 | 7435806 | BLASTX | 255 | 1.00E−21 | 42 | cysteine proteinase mir3 (EC 3.4.22.—) - maize |
| | | | | | | | >gi\|2425066\|gb\|AAB88263.1\| (AF019147) cysteine |
| | | | | | | | proteinase Mir3 [Zea mays] |
| 2106 | LIB3602-064-Q1-K6-B11 | 3334112 | BLASTX | 279 | 2.00E−24 | 60 | ACYL-COA-BINDING PROTEIN (ACBP) |
| | | | | | | | >gi\|7441625\|pir\|\|T09844 acyl-CoA-binding protein - |
| | | | | | | | castor bean >gi\|1938236\|emb\|CAA70200.1\| |
| | | | | | | | (Y08996) acyl-CoA-binding protein [Ricinus |
| | | | | | | | communis] |
| 2107 | LIB3602-011-Q6-K1-H5 | 3334112 | BLASTX | 278 | 2.00E−24 | 60 | ACYL-COA-BINDING PROTEIN (ACBP) |
| | | | | | | | >gi\|7441625\|pir\|\|T09844 acyl-CoA-binding protein - |
| | | | | | | | castor bean >gi\|1938236\|emb\|CAA70200.1\| |
| | | | | | | | (Y08996) acyl-CoA-binding protein [Ricinus |
| | | | | | | | communis] |
| 2108 | LIB3602-078-Q6-K6-F9 | 6498428 | BLASTX | 684 | 6.00E−72 | 69 | (AP000815) Similar to Arabidopsis thaliana |
| | | | | | | | chromosome II BAC T27A16 sequence; |
| | | | | | | | hypothetical protein. (AC005496) [Oryza sativa] |
| 2109 | LIB3602-072-Q1-K1-E5 | 6498428 | BLASTX | 648 | 1.00E−67 | 68 | (AP000815) Similar to Arabidopsis thaliana |
| | | | | | | | chromosome II BAC T27A16 sequence; |
| | | | | | | | hypothetical protein. (AC005496) [Oryza sativa] |
| 2110 | LIB3602-041-Q6-K1-A4 | 7549645 | BLASTX | 199 | 1.00E−15 | 82 | (AC023912) ribosomal protein L29, putative |
| | | | | | | | [Arabidopsis thaliana] |
| 2111 | LIB3602-010-Q6-K1-H9 | 7549645 | BLASTX | 199 | 2.00E−15 | 82 | (AC023912) ribosomal protein L29, putative |
| | | | | | | | [Arabidopsis thaliana] |
| 2112 | LIB3602-074-Q1-K1-C9 | 3153873 | BLASTX | 829 | 6.00E−89 | 77 | (AF065393) putative G-binding protein [Homo |
| | | | | | | | sapiens] |
| 2113 | LIB3602-107-Q1-K1-B12 | 3153873 | BLASTX | 773 | 2.00E−82 | 79 | (AF065393) putative G-binding protein [Homo |
| | | | | | | | sapiens] |
| 2114 | LIB3602-027-Q6-K1-A6 | 7271018 | BLASTX | 274 | 5.00E−24 | 54 | (AJ390506) ribosomal protein L16 [Candida |
| | | | | | | | albicans] |
| 2115 | LIB3602-049-Q6-K1-C12 | 7271018 | BLASTX | 202 | 1.00E−15 | 50 | (AJ390506) ribosomal protein L16 [Candida |
| | | | | | | | albicans] |
| 2116 | LIB3602-041-Q6-K1-A3 | 7469371 | BLASTX | 654 | 2.00E−68 | 60 | hypothetical protein - Synechocystis sp. (strain PCC |
| | | | | | | | 6803) >gi\|1001311\|dbj\|BAA10798.1\| (D64006) |
| | | | | | | | hypothetical protein [Synechocystis sp.] |
| 2117 | LIB3602-041-Q6-K1-A8 | 7469371 | BLASTX | 199 | 4.00E−15 | 52 | hypothetical protein - Synechocystis sp. (strain PCC |
| | | | | | | | 6803) >gi\|1001311\|dbj\|BAA10798.1\| (D64006) |
| | | | | | | | hypothetical protein [Synechocystis sp.] |
| 2118 | LIB3602-091-Q6-K6-B11 | 7484843 | BLASTX | 397 | 3.00E−38 | 66 | cell division protein ftsY homolog F4I18.25 - |
| | | | | | | | Arabidopsis thaliana >gi\|3386617\|gb\|AAC28547.1\| |
| | | | | | | | (AC004665) putative signal recognition particle |
| | | | | | | | receptor (alpha subunit) [Arabidopsis thaliana] |
| 2119 | LIB3602-094-Q6-K6-B11 | 7484843 | BLASTX | 285 | 3.00E−25 | 54 | cell division protein ftsY homolog F4I18.25 - |
| | | | | | | | Arabidopsis thaliana >gi\|3386617\|gb\|AAC28547.1\| |
| | | | | | | | (AC004665) putative signal recognition particle |
| | | | | | | | receptor (alpha subunit) [Arabidopsis thaliana] |
| 2120 | LIB3602-093-Q6-K6-H4 | 729396 | BLASTX | 278 | 2.00E−24 | 36 | ELONGATION FACTOR 1-ALPHA (EF-1- |
| | | | | | | | ALPHA) (ELONGATION FACTOR TU) (EF-TU) |
| | | | | | | | >gi\|581023\|emb\|CAA51984.1\| (X73582) elongation |
| | | | | | | | factor 1-alpha [Desulfurococcus mobilis] |
| 2121 | LIB3602-101-Q1-K1-C3 | 729396 | BLASTX | 226 | 2.00E−18 | 37 | ELONGATION FACTOR 1-ALPHA (EF-1- |
| | | | | | | | ALPHA) (ELONGATION FACTOR TU) (EF-TU) |
| | | | | | | | >gi\|581023\|emb\|CAA51984.1\| (X73582) elongation |
| | | | | | | | factor 1-alpha [Desulfurococcus mobilis] |
| 2122 | LIB3602-038-Q6-K1-D11 | 2494208 | BLASTX | 265 | 5.00E−23 | 58 | DYNEIN BETA CHAIN, FLAGELLAR OUTER |
| | | | | | | | ARM >gi\|7484362\|pir\|\|T08030 dynein beta heavy |
| | | | | | | | chain - Chlamydomonas reinhardtii |
| | | | | | | | >gi\|514215\|gb\|AAA19956.1\| (U02963) dynein beta |
| | | | | | | | heavy chain [Chlamydomonas reinhardtii] |
| 2123 | LIB3602-038-Q6-K1-D12 | 2494208 | BLASTX | 153 | 5.00E−10 | 40 | DYNEIN BETA CHAIN, FLAGELLAR OUTER |
| | | | | | | | ARM >gi\|7484362\|pir\|\|T08030 dynein beta heavy |
| | | | | | | | chain - Chlamydomonas reinhardtii |
| | | | | | | | >gi\|514215\|gb\|AAA19956.1\| (U02963) dynein beta |
| | | | | | | | heavy chain [Chlamydomonas reinhardtii] |
| 2124 | LIB3602-072-Q1-K1-D11 | 4836948 | BLASTX | 526 | 2.00E−53 | 64 | (AC006085) Similar to human CGI-33 protein |
| | | | | | | | [Arabidopsis thaliana] |
| 2125 | LIB3602-028-Q6-K1-B7 | 4836948 | BLASTX | 367 | 7.00E−35 | 63 | (AC006085) Similar to human CGI-33 protein |
| | | | | | | | [Arabidopsis thaliana] |
| 2126 | LIB3602-047-Q6-K1-F2 | 3023740 | BLASTX | 367 | 7.00E−35 | 75 | FERREDOXIN >gi\|7430737\|pir\|\|A68399 ferredoxin |
| | | | | | | | [2Fe-2S] - Chlorella fusca >gi\|2914662\|pdb\|1AWD\| |
| | | | | | | | Ferredoxin [2fe-2s] Oxidized Form From Chlorella |
| | | | | | | | Fusca |
| 2127 | LIB3602-029-Q6-K1-A12 | 3023740 | BLASTX | 340 | 9.00E−32 | 82 | FERREDOXIN >gi\|7430737\|pir\|\|A68399 ferredoxin |
| | | | | | | | [2Fe-2S] - Chlorella fusca >gi\|2914662\|pdb\|1AWD\| |
| | | | | | | | Ferredoxin [2fe-2s] Oxidized Form From Chlorella |
| | | | | | | | Fusca |
| 2128 | LIB3602-003-Q1-K1-D5 | 417744 | BLASTX | 635 | 3.00E−66 | 74 | ADENOSYLHOMOCYSTEINASE (S- |
| | | | | | | | ADENOSYL-L-HOMOCYSTEINE |
| | | | | | | | HYDROLASE) (ADOHCYASE) |
| | | | | | | | >gi\|169663\|gb\|AAA33856.1\| (M81885) S- |
| | | | | | | | adenosylhomocysteine hydrolase [Petroselinum |
| | | | | | | | crispum] |
| 2129 | LIB3602-115-Q1-K1-G10 | 417744 | BLASTX | 492 | 7.00E−62 | 71 | ADENOSYLHOMOCYSTEINASE (S- |
| | | | | | | | ADENOSYL-L-HOMOCYSTEINE |
| | | | | | | | HYDROLASE) (ADOHCYASE) |
| | | | | | | | >gi\|169663\|gb\|AAA33856.1\| (M81885) S- |
| | | | | | | | adenosylhomocysteine hydrolase [Petroselinum |
| | | | | | | | crispum] |
| 2130 | LIB3602-064-Q1-K6-E11 | 1172811 | BLASTX | 479 | 4.00E−48 | 89 | 60S RIBOSOMAL PROTEIN L10-1 (PUTATIVE |
| | | | | | | | TUMOR SUPRESSOR SC34) |
| | | | | | | | >gi\|1076751\|pir\|S49575 ribosomal protein L10.e, |
| | | | | | | | cytosolic - rice >gi\|575355\|emb\|CAA57339.1\| |
| | | | | | | | (X81691) putative tumor suppresser [Oryza sativa] |
| 2131 | LIB3602-117-Q1-K1-E9 | 1172811 | BLASTX | 195 | 1.00E−27 | 87 | 60S RIBOSOMAL PROTEIN L10-1 (PUTATIVE |
| | | | | | | | TUMOR SUPRESSOR SC34) |
| | | | | | | | >gi\|1076751\|pir\|\|S549575 ribosomal protein L10.e, |
| | | | | | | | cytosolic - rice >gi\|575355\|emb\|CAA57339.1\| |
| | | | | | | | (X81691) putative tumor suppresser [Oryza sativa] |
| 2132 | LIB3602-040-Q6-K1-B11 | 6319773 | BLASTX | 235 | 2.00E−19 | 36 | Probable Na+/Pi symporter; Pho89p |
| | | | | | | | >gi\|586363\|sp\|P38361\|YB81_YEAST PUTATIVE |
| | | | | | | | PHOSPHATE-REPRESSIBLE PHOSPHATE |
| | | | | | | | PERMEASE YBR29C >gi\|626777\|pir\|\|S46178 |
| | | | | | | | phosphate-repressible phosphate permease homolog |
| | | | | | | | YBR296c - yeast (Saccharomyces cerevisiae) |
| | | | | | | | >gi\|536756\|emb\|CA |
| 2133 | LIB3602-053-Q6-K1-H12 | 6319773 | BLASTX | 221 | 6.00E−18 | 36 | Probable Na+/Pi symporter; Pho89p |
| | | | | | | | >gi\|586363\|sp\|P38361\|YB8I_YEAST PUTATIVE |
| | | | | | | | PHOSPHATE-REPRESSIBLE PHOSPHATE |
| | | | | | | | PERMEASE YBR29C >gi\|626777\|pir\|\|S46178 |
| | | | | | | | phosphate-repressible phosphate permease homolog |
| | | | | | | | YBR296c - yeast (Saccharomyces cerevisiae) |
| | | | | | | | >gi\|536756\|emb\|CA |
| 2134 | LIB3602-106-Q1-K1-F10 | 7322532 | BLASTX | 206 | 7.00E−16 | 45 | (AC024756) contains similarity to |
| | | | | | | | SW: GLRX_RABIT [Caenorhabditis elegans] |
| 2135 | LIB3602-100-Q1-K1-F10 | 7322532 | BLASTX | 205 | 7.00E−16 | 46 | (AC024756) contains similarity to |
| | | | | | | | SW: GLRX_RABIT [Caenorhabditis elegans] |
| 2136 | LIB3602-047-Q6-K1-C6 | 5714433 | BLASTX | 325 | 5.00E−30 | 41 | (AF120112) chloroplast SRP receptor homolog, |
| | | | | | | | alpha subunit CPFTSY; chloroplast signal |
| | | | | | | | recognition particle receptor alpha homolog |
| | | | | | | | CPFTSY [Arabidopsis thaliana] |
| 2137 | LIB3602-066-Q1-K6-E3 | 5714433 | BLASTX | 202 | 2.00E−15 | 44 | (AF120112) chloroplast SRP receptor homolog, |
| | | | | | | | alpha subunit CPFTSY; chloroplast signal |
| | | | | | | | recognition particle receptor alpha homolog |
| | | | | | | | CPFTSY [Arabidopsis thaliana] |
| 2138 | LIB3602-047-Q6-K1-F4 | 6644196 | BLASTX | 193 | 2.00E−14 | 86 | (AF207690) chlorophyll a/b-binding protein |
| | | | | | | | [Daucus carota] |
| 2139 | LIB3602-059-Q6-K1-H9 | 6644196 | BLASTX | 193 | 2.00E−14 | 86 | (AF207690) chlorophyll a/b-binding protein |
| | | | | | | | [Daucus carota] |
| 2140 | LIB3602-065-Q1-K6-H11 | 4835754 | BLASTX | 624 | 6.00E−65 | 76 | (AC007202) Is a member of the PF\|00162 |
| | | | | | | | Phosphoglycerate kinase family. ESTs gb\|N38721, |
| | | | | | | | gb\|T22178, gb\|R90345, gb\|R90715, gb\|T21140, |
| | | | | | | | gb\|T46295, gb\|H37082, gb\|T46076, gb\|N37132, |
| | | | | | | | gb\|AA597649, gb\|AI100648 and gb\|Z48462 come |
| | | | | | | | from this gene. [Arabidopsis tha> |
| 2141 | LIB3602-057-Q6-K1-F2 | 4835754 | BLASTX | 542 | 2.00E−55 | 67 | (AC007202) Is a member of the PF\|00162 |
| | | | | | | | Phosphoglycerate kinase family. ESTs gb\|N38721, |
| | | | | | | | gb\|T22178, gb\|R90345, gb\|R90715, gb\|T21140, |
| | | | | | | | gb\|T46295, gb\|H37082, gb\|T46076, gb\|N37132, |
| | | | | | | | gb\|AA597649, gb\|AI100648 and gb\|Z48462 come |
| | | | | | | | from this gene. [Arabidopsis tha> |
| 2142 | LIB3602-083-Q6-K6-E4 | 7385201 | BLASTX | 632 | 8.00E−66 | 70 | (AF243182) beta-ketoacyl-ACP synthetase I |
| | | | | | | | [Glycine max] |
| 2143 | LIB3602-062-Q6-K1-E3 | 7331195 | BLASTX | 149 | 3.00E−09 | 33 | (AF236108) putative purple acid phosphatase |
| | | | | | | | precursor [Glycine max] |
| 2144 | LIB3602-068-Q1-K1-H1 | 5441551 | BLASTX | 362 | 2.00E−34 | 63 | (AJ388528) Ribosomal protein [Canis familiaris] |
| 2145 | LIB3602-063-Q1-K6-A2 | 7300833 | BLASTX | 314 | 1.00E−28 | 37 | (AE003738) CG5383 gene product [Drosophila |
| | | | | | | | melanogaster] |
| 2146 | LIB3602-038-Q6-K1-F5 | 7487011 | BLASTX | 193 | 2.00E−14 | 36 | hypothetical protein T14P8.20 - Arabidopsis |
| | | | | | | | thaliana >gi\|3193300\|gb\|AAC19284.1\| (AF069298) |
| | | | | | | | T14P8.20 gene product [Arabidopsis thaliana] |
| | | | | | | | >gi\|7269000\|emb\|CAB80733.1\| (AL161494) |
| | | | | | | | contains EST gb: AI998867.1 [Arabidopsis thaliana] |
| 2147 | LIB3602-042-Q6-K1-E7 | 5478530 | BLASTX | 261 | 2.00E−22 | 40 | (AF130441) UVB-resistance protein UVR8 |
| | | | | | | | [Arabidopsis thaliana] |
| 2148 | LIB3602-074-Q1-K1-A11 | 7108615 | BLASTX | 380 | 4.00E−41 | 63 | AF130845) homogentisate 1,2-dioxygenase |
| | | | | | | | [Arabidopsis thaliana] |
| 2149 | LIB3602-086-Q6-K1-G7 | 6912654 | BLASTX | 456 | 3.00E−45 | 56 | splicing factor 3b, subunit 1, 155 kD |
| | | | | | | | >gi\|4033735\|gb\|AAC97189.1\| (AF054284) |
| | | | | | | | spliceosomal protein SAP 155 [Homo sapiens] |
| 2150 | LIB3602-104-Q1-K1-G1 | 477724 | BLASTX | 221 | 1.00E−17 | 73 | acetyl-CoA synthetase homolog - Escherichia coli |
| | | | | | | | (fragment) >gi\|148105\|gb\|AAA24715.1\| (M87509) |
| | | | | | | | acetyl-CoA synthetase [Escherichia coli] |
| 2151 | LIB3602-051-Q6-K1-B3 | 4874275 | BLASTX | 403 | 4.00E−39 | 53 | (AC007354) Similar to gb\|L26291 clathrin- |
| | | | | | | | associated protein unc-101 from Caenorhabditis |
| | | | | | | | elegans and is a member of the PF\|00928 Adapter |
| | | | | | | | complexes medium subunit family. [Arabidopsis |
| | | | | | | | thaliana] |
| 2152 | LIB3602-086-Q6-K1-A12 | 7485223 | BLASTX | 309 | 5.00E−28 | 62 | hypothetical protein A_IG002N01.18 - Arabidopsis |
| | | | | | | | thaliana >gi\|2191138\|gb\|AAB61025.1\| (AF007269) |
| | | | | | | | A_IG002N01.18 gene product [Arabidopsis |
| | | | | | | | thaliana]>gi\|7267612\|emb\|CAB80924.1\| |
| | | | | | | | (AL161491) hypothetical protein [Arabidopsis |
| | | | | | | | thaliana] |
| 2153 | LIB3602-015-Q6-K1-F6 | 5729802 | BLASTX | 601 | 3.00E−62 | 87 | similar to S. pombe dim1+ |
| | | | | | | | >gi\|6225270\|sp\|O14834\|DIM1_HUMAN DIM1 |
| | | | | | | | PROTEIN HOMOLOG >gi\|6730460\|pdb\|1QGV\|A |
| | | | | | | | Chain A, Human Spliceosomal Protein U5-15 kd |
| | | | | | | | >gi\|2565275\|gb\|AAB81950.1\| (AF023611) Dim1p |
| | | | | | | | homolog [Homo sapiens] |
| | | | | | | | >gi\|6572636\|gb\|AAF17332.1\|AF146373_1 (AF1 |
| 2154 | LIB3602-045-Q6-K1-H6 | 4506729 | BLASTX | 162 | 4.00E−11 | 35 | ribosomal protein S5 |
| | | | | | | | >gi\|1173267\|sp\|P46782\|RS5_HUMAN 40S |
| | | | | | | | RIBOSOMAL PROTEIN S5 |
| | | | | | | | >gi\|1362935\|pir\|\|S55916 ribosomal protein S5, |
| | | | | | | | cytosolic - human >gi\|550021\|gb\|AAA85658.1\| |
| | | | | | | | (U14970) ribosomal protein S5 [Homo sapiens] |
| | | | | | | | >gi\|1096942\|prf\|\|2113200E ribosomal protei |
| 2155 | LIB3602-020-Q6-K1-C11 | 6681153 | BLASTX | 255 | 9.00E−22 | 50 | dolichyl-di-phosphooligosaccharide-protein |
| | | | | | | | glycotransferase >gi\|2662377\|dbj\|BAA23671.1\| |
| | | | | | | | (D89063) oligosaccharyltransferase [Mus musculus] |
| 2156 | LIB3602-023-Q6-K1-A4 | 6041851 | BLASTX | 155 | 6.00E−10 | 56 | (AC009853) unknown protein [Arabidopsis |
| | | | | | | | thaliana] |
| 2157 | LIB3602-006-Q1-K1-F8 | 7293667 | BLASTX | 232 | 7.00E−19 | 40 | (AE003514) CG6597 gene product [Drosophila |
| | | | | | | | melanogaster] |
| 2158 | LIB3602-019-Q6-K1-C11 | 6652878 | BLASTX | 645 | 1.00E−67 | 92 | (AF123390) 26S proteasome AAA-ATPase subunit |
| | | | | | | | RPT1a [Arabidopsis thaliana] |
| 2159 | LIB3602-076-Q6-K6-B2 | 6322518 | BLASTX | 197 | 6.00E−15 | 59 | Clathrin-associated protein, small subunit; Aps2p |
| | | | | | | | >gi\|231554\|sp\|Q00381\|AP17_YEAST CLATHRIN |
| | | | | | | | COAT ASSEMBLY PROTEIN AP17 (CLATHRIN |
| | | | | | | | COAT ASSOCIATED PROTEIN AP17) |
| | | | | | | | (PLASMA MEMBRANE ADAPTOR AP-2 17 KD |
| | | | | | | | PROTEIN) (HA2 17 KD SUBUNIT) (CLATHRIN |
| | | | | | | | ASSEMBLY PROTEIN 2 SMA |
| 2160 | LIB3602-001-P1-K6-E2 | 2497984 | BLASTX | 218 | 3.00E−17 | 31 | MITOCHONDRIAL |
| | | | | | | | CARNITINE/ACYLCARNITINE CARRIER |
| | | | | | | | PROTEIN (CARNITINE/ACYLCARNITINE |
| | | | | | | | TRANSLOCASE) (CAC) |
| | | | | | | | >gi\|1842211\|emb\|CAA66410.1\| (X97831) |
| | | | | | | | carnitine/acylcarnitine carrier protein [Rattus |
| | | | | | | | norvegicus] |
| 2161 | LIB3602-089-Q6-K6-B2 | 7025513 | BLASTX | 167 | 2.00E−11 | 54 | (AF233593) ascorbate oxidase AO1 [Cucumis |
| | | | | | | | melo] |
| 2162 | LIB3602-101-Q1-K1-G1 | 6840800 | BLASTX | 247 | 1.00E−30 | 60 | ADENOSINE KINASE (AK) (ADENOSINE 5′- |
| | | | | | | | PHOSPHOTRANSFERASE) |
| 2163 | LIB3602-077-Q6-K6-A5 | 6525009 | BLASTX | 169 | 1.00E−11 | 46 | (AF198626) copper chaperone homolog CCH |
| | | | | | | | [Oryza sativa] |
| 2164 | LIB3602-077-Q6-K6-G5 | 4836473 | BLASTX | 218 | 3.00E−17 | 45 | (AF123257) 17.6 kD class I small heat shock |
| | | | | | | | protein [Lycopersicon esculentum] |
| 2165 | LIB3602-038-Q6-K1-F6 | 7340070 | BLASTX | 412 | 9.00E−45 | 70 | (AF220552) ribosomal protein L17 [Paralichthys |
| | | | | | | | olivaceus] |
| 2166 | LIB3602-050-Q6-K1-C5 | 6735351 | BLASTX | 433 | 1.00E−42 | 57 | (AL137081) pelota-like protein [Arabidopsis |
| | | | | | | | thaliana] |
| 2167 | LIB3602-003-Q1-K1-E7 | 7302653 | BLASTX | 191 | 3.00E−14 | 52 | (AE003800) Dgp-1 gene product [Drosophila |
| | | | | | | | melanogaster] |
| 2168 | LIB3602-090-Q6-K6-F9 | 6325229 | BLASTX | 204 | 1.00E−15 | 54 | acetoacetyl CoA thiolase; Erg10p |
| | | | | | | | >gi\|730943\|sp\|P41338\|THIL_YEAST ACETYL- |
| | | | | | | | COA ACETYLTRANSFERASE |
| | | | | | | | (ACETOACETYL-COA THIOLASE) |
| | | | | | | | >gi\|1071833\|pir\|\|A55654 acetyl-CoA C- |
| | | | | | | | acetyltransferase (EC 2.3.1.9), cytosolic - yeast |
| | | | | | | | (Saccharomyces cerevisiae) >gi\|311089\|gb\|AAA623 |
| 2169 | LIB3602-087-Q6-K1-B6 | 2500430 | BLASTX | 467 | 6.00E−49 | 70 | 40S RIBOSOMAL PROTEIN S16 |
| 2170 | LIB3602-051-Q6-K1-D4 | 7437805 | BLASTX | 602 | 5.00E−68 | 77 | phenylalanine-tRNA ligase homolog T22F8.180 - |
| | | | | | | | Arabidopsis thaliana |
| | | | | | | | >gi\|4914440\|emb\|CAB43643.1\| (AL050351) |
| | | | | | | | phenylalanyl-trna synthetase-like protein |
| | | | | | | | [Arabidopsis thaliana] |
| | | | | | | | >gi\|7270911\|emb\|CAB80591.1\| (AL161594) |
| | | | | | | | phenylalanyl-trna synthetase-like protein [Ar |
| 2171 | LIB3602-028-Q6-K1-B3 | 5923675 | BLASTX | 347 | 2.00E−32 | 42 | (AC009326) putative mRNA capping enzyme, RNA |
| | | | | | | | guanylyltransferase [Arabidopsis thaliana] |
| 2172 | LIB3602-029-Q6-K1-B2 | 7491269 | BLASTX | 149 | 3.00E−09 | 44 | hypothetical protein SPAC8C9.11 - fission yeast |
| | | | | | | | (Schizosaccharomyces pombe) |
| | | | | | | | >gi\|2408094\|emb\|CAB16299.1\| (Z99168) |
| | | | | | | | hypothetical protein [Schizosaccharomyces pombe] |
| 2173 | LIB3602-068-Q1-K1-B11 | 7267629 | BLASTX | 229 | 9.00E−19 | 54 | (AL161491) putative CAAX prenyl protease |
| | | | | | | | [Arabidopsis thaliana] |
| 2174 | LIB3602-066-Q1-K6-H9 | 4558669 | BLASTX | 328 | 4.00E−30 | 50 | (AC007063) putative nucleotide-sugar transporter |
| | | | | | | | [Arabidopsis thaliana] |
| 2175 | LIB3602-023-Q6-K1-F4 | 2996012 | BLASTX | 627 | 3.00E−65 | 68 | (AF054455) cytosolic phosphoglucose isomerase; |
| | | | | | | | PgiC [Leavenworthia crassa] |
| 2176 | LIB3602-090-Q6-K6-B1 | 7259294 | BLASTX | 254 | 2.00E−21 | 42 | (AB030200) expression of this gene product |
| | | | | | | | inhibits the growth of E. coli [Mus musculus] |
| 2177 | LIB3602-084-Q6-K1-B5 | 6755114 | BLASTX | 331 | 1.00E−30 | 48 | peroxisomal membrane protein 20 |
| | | | | | | | >gi\|6166491\|gb\|AAF04855.1\|AF197951_1 |
| | | | | | | | (AF197951) thioredoxin peroxidase PMP20 [Mus |
| | | | | | | | musculus] |
| 2178 | LIB3602-063-Q1-K6-C8 | 3183373 | BLASTX | 225 | 3.00E−18 | 48 | HYPOTHETICAL 28.3 KD PROTEIN C17A5.05C |
| | | | | | | | IN CHROMOSOME I >gi\|7490843\|pir\|\|T37820 |
| | | | | | | | hypothetical protein SPAC17A5.05c - fission yeast |
| | | | | | | | (Schizosaccharomyces pombe) |
| | | | | | | | >gi\|2370477\|emb\|CAB11505.1\| (Z98849) |
| | | | | | | | hypothetical protein [Schizosaccharomyces pombe] |
| 2179 | LIB3602-109-Q1-K1-H6 | 7329687 | BLASTX | 496 | 4.00E−55 | 83 | (AL162295) ribosomal protein S13-like |
| | | | | | | | [Arabidopsis thaliana] |
| 2180 | LIB3602-011-Q6-K1-G7 | 6751711 | BLASTX | 383 | 1.00E−36 | 50 | (AC018848) putative aspartate aminotransferase |
| | | | | | | | [Arabidopsis thaliana] |
| 2181 | LIB3602-012-Q6-K1-E8 | 6751712 | BLASTX | 287 | 2.00E−25 | 47 | (AC018848) unknown protein [Arabidopsis |
| | | | | | | | thaliana] |
| 2182 | LIB3602-011-Q6-K1-B5 | 2706555 | BLASTX | 593 | 2.00E−61 | 67 | (Y15942) succinate dehydrogenase iron-sulfur |
| | | | | | | | subunit [Agaricus bisporus] |
| 2183 | LIB3602-010-Q6-K1-E8 | 7302498 | BLASTX | 304 | 8.00E−28 | 62 | (AE003796) FK506 [symbol] gene product [Drosophila |
| | | | | | | | melanogaster |
| 2184 | LIB3602-039-Q6-K1-C6 | 7546515 | BLASTX | 474 | 2.00E−47 | 72 | Chain A, Human Translation Initiation Factor Eif1a |
| 2185 | LIB3602-048-Q6-K1QA-E3 | 6573706 | BLASTX | 465 | 2.00E−46 | 56 | (AC009978) T23E18.6 [Arabidopsis thaliana] |
| 2186 | LIB3602-115-Q1-K1-H10 | 6573708 | BLASTX | 234 | 2.00E−19 | 66 | (AC009978) T23E18.8 [Arabidopsis thaliana] |
| 2187 | LIB3602-113-Q1-K1-E6 | 6735373 | BLASTX | 447 | 3.00E−54 | 58 | (AL137082) putative protein [Arabidopsis thaliana] |
| 2188 | LIB3602-057-Q6-K1-G7 | 6323420 | BLASTX | 197 | 5.00E−15 | 76 | Ribosomal protein S29A (S36A) (YS29); Rps29ap |
| | | | | | | | >gi\|730461\|sp\|P41057\|R29A_YEAST 40S |
| | | | | | | | RIBOSOMAL PROTEIN S29-A (S36) (YS29) |
| | | | | | | | >gi\|626904\|pir\|\|S48503 ribosomal protein S29.e.A, |
| | | | | | | | cytosolic - yeast (Saccharomyces cerevisiae) |
| | | | | | | | >gi\|287628\|dbj\|BAA03507.1\| (D14676) ribos |
| 2189 | LIB3602-051-Q6-K1-D8 | 7385055 | BLASTX | 193 | 2.00E−14 | 42 | (AF216387) beta-adaptin-like protein C |
| | | | | | | | [Arabidopsis thaliana] |
| 2190 | LIB3602-116-Q1-K1-E4 | 7304097 | BLASTX | 355 | 2.00E−33 | 54 | (AE003838) CG8711 gene product [Drosophila |
| | | | | | | | melanogaster] |
| 2191 | LIB3602-027-Q6-K1-F1 | 3687243 | BLASTX | 219 | 1.00E−17 | 82 | (AC005169) 40S ribosomal protein S30 |
| | | | | | | | [Arabidopsis thaliana] |
| 2192 | LIB3602-078-Q6-K6-B10 | 7446384 | BLASTX | 327 | 4.00E−30 | 41 | probable protein kinase - tomato |
| | | | | | | | >gi\|237025 [symbol] 722.1\| (Y13273) putative |
| | | | | | | | protein kin [symbol] icon esculentum] |
| | | | | | | | >gi\|5669642\|gb\|AAD [symbol] 406.1\|AF096250_1 |
| | | | | | | | (AF096250) ethylene-responsive protein kinase |
| | | | | | | | TCTR1 [Lycopersicon esculentum] |
| 2193 | LIB3602-117-Q1-K1-G11 | 7487986 | BLASTX | 281 | 6.00E−35 | 49 | outer envelope membrane protein homolog |
| | | | | | | | T6H20.230 - Arabidopsis thaliana |
| | | | | | | | >gi\|5541685\|emb\|CAB51191.1\| (AL096859) |
| | | | | | | | chloroplast import-associated channel homolog |
| | | | | | | | [Arabidopsis thaliana] |
| 2194 | LIB3602-009-Q6-K1-D5 | 3283368 | BLASTX | 317 | 6.00E−29 | 56 | (AF067082) mannitol dehydrogenase [Apium |
| | | | | | | | graveolens] |
| 2195 | LIB3602-058-Q6-K1-B1 | 7076772 | BLASTX | 283 | 4.00E−25 | 47 | (AL138658) dynamin-like protein 4 (ADL4) |
| | | | | | | | [Arabidopsis thaliana] |
| 2196 | LIB3602-093-Q6-K6-H8 | 7294336 | BLASTX | 429 | 4.00E−42 | 58 | (AE003532) Prosbeta2 gene product [Drosophila |
| | | | | | | | melanogaster] |
| 2197 | LIB3602-073-Q1-K1-C2 | 7076779 | BLASTX | 529 | 8.00E−54 | 55 | (AL132975) glycoprotein-like [Arabidopsis |
| | | | | | | | thaliana] |
| 2198 | LIB3602-116-Q1-K1-D11 | 2129893 | BLASTX | 191 | 1.00E−21 | 87 | preprotein translocase secA precursor - garden pea |
| 2199 | LIB3602-011-Q6-K1-D4 | 7445481 | BLASTX | 336 | 3.00E−31 | 54 | hypothetical protein F17M5.230 - Arabidopsis |
| | | | | | | | thaliana>gi\|4490314\|emb\|CAB38805.1\| |
| | | | | | | | (AL035678) putative protein [Arabidopsis thaliana] |
| | | | | | | | >gi\|7270295\|emb\|CAB80064.1\| (AL161583) |
| | | | | | | | putative protein [Arabidopsis thaliana] |
| 2200 | LIB3602-045-Q6-K1-H1 | 5730023 | BLASTX | 629 | 1.00E−65 | 95 | RuvB (E coli homolog)-like 2 |
| | | | | | | | >gi\|4587311\|dbj\|BAA76708.1\| (AB024301) RuvB- |
| | | | | | | | like DNA helicase TIP49b [Homo sapiens] |
| | | | | | | | >gi\|5020422\|gb\|AAD38073.1\|AF155138_1 |
| | | | | | | | (AF155138) RUVBL2 protein [Homo sapiens] |
| | | | | | | | >gi\|5326998\|emb\|CAB46270.1\| (Y18417) |
| | | | | | | | erythrocyte cytosolic prote |
| 2201 | LIB3602-059-Q6-K1-H3 | 5080810 | BLASTX | 457 | 2.00E−45 | 46 | (AC007258) Very similar to helicases [Arabidopsis |
| | | | | | | | thaliana] |
| 2202 | LIB3602-092-Q6-K6-B8 | 6449503 | BLASTN | 35 | 5.00E−10 | 92 | Genomic Sequence For Arabidopsis thaliana Clone |
| | | | | | | | F15F15, Chromosome V, complete sequence |
| 2203 | LIB3602-040-Q6-K1-D3 | 2760327 | BLASTX | 481 | 3.00E−48 | 72 | (AC002130) F1N21.12 [Arabidopsis thaliana] |
| 2204 | LIB3602-042-Q6-K1-A11 | 1708463 | BLASTX | 205 | 8.00E−16 | 31 | IAA-AMINO ACID HYDROLASE |
| | | | | | | | >gi\|887785\|gb\|AAB60293.1\| (U23794) ILR1 |
| | | | | | | | [Arabidopsis thaliana] |
| 2205 | LIB3602-026-Q6-K1-G3 | 586059 | BLASTX | 697 | 2.00E−73 | 74 | LYSYL--TRNA SYNTHETASE (LYSINE-TRNA |
| | | | | | | | LIGASE) (LYSRS) >gi\|543180\|pir\|\|S43187 lysine-- |
| | | | | | | | tRNA ligase (EC 6.1.1.6) - long-tailed hamster |
| | | | | | | | >gi\|469122\|emb\|CAA83505.1\| (Z31711) Lysyl |
| | | | | | | | tRNA Synthetase [Cricetulus longicaudatus] |
| 2206 | LIB3602-068-Q1-K1-A7 | 3980394 | BLASTX | 177 | 2.00E−12 | 41 | (AC004561) putative zinc transporter [Arabidopsis |
| | | | | | | | thaliana] |
| 2207 | LIB3602-032-Q6-K1-C1 | 6553927 | BLASTX | 190 | 2.00E−14 | 86 | (AC012329) putative alpha NAC [Arabidopsis |
| | | | | | | | thaliana] >gi\|6561948\|emb\|CAB62452.1\| |
| | | | | | | | (AL132964) alpha NAC-like protein [Arabidopsis |
| | | | | | | | thaliana] |
| 2208 | LIB3602-087-Q6-K1-G11 | 7329665 | BLASTX | 329 | 9.00E−34 | 43 | (AL162351) putative protein [Arabidopsis thaliana] |
| 2209 | LIB3602-054-Q6-K1-H12 | 4557765 | BLASTX | 475 | 1.00E−47 | 59 | 5-methyltetrahydrofolate-homocysteine |
| | | | | | | | methyltransferase >gi\|2160699\|gb\|AAB58906.1\| |
| | | | | | | | (U75743) methionine synthase [Homo sapiens] |
| 2210 | LIB3602-091-Q6-K6-B9 | 4826686 | BLASTX | 388 | 3.00E−37 | 57 | DEAD/H (Asp-Glu-Ala-Asp/His) box polypeptide 1 |
| | | | | | | | >gi\|6919862\|sp\|Q92499\|DDX1_HUMAN DEAD |
| | | | | | | | BOX PROTEIN 1 (DEAD BOX PROTEIN- |
| | | | | | | | RETINOBLASTOMA) (DBP-RB) |
| | | | | | | | >gi\|3123574\|emb\|CAA49992.1\| (X70649) member |
| | | | | | | | of DEAD box protein family [Homo sapiens] |
| 2211 | LIB3602-116-Q1-K1-A3 | 4506701 | BLASTX | 235 | 2.00E−26 | 89 | ribosomal protein S23 |
| | | | | | | | >gi\|730647\|sp\|P39028\|RS23_HUMAN 40S |
| | | | | | | | RIBOSOMAL PROTEIN S23 |
| | | | | | | | >gi\|543449\|pir\|\|S41955 ribosomal protein S23, |
| | | | | | | | cytosolic - rat >gi\|631360\|pir\|\|S42105 ribosomal |
| | | | | | | | protein S23, cytosolic - human |
| | | | | | | | >gi\|414349\|dbj\|BAA03400.1\| (D14530) ribosomal |
| | | | | | | | pro |
| 2212 | LIB3602-072-Q1-K1-E8 | 6322576 | BLASTX | 186 | 1.00E−13 | 32 | Yjr116wp >gi\|1352917\|sp\|P47153\|YJ86_YEAST |
| | | | | | | | HYPOTHETICAL 32.0 KD PROTEIN IN NNF1- |
| | | | | | | | STE24 INTERGENIC REGION |
| | | | | | | | >gi\|1078275\|pir\|\|S57139 probable membrane |
| | | | | | | | protein YJR116w - yeast (Saccharomyces |
| | | | | | | | cerevisiae) >gi\|1015835\|emb\|CAA89646.1\| |
| | | | | | | | (Z49616) ORF YJR116w [Saccharom |
| 2213 | LIB3602-056-Q6-K1-G11 | 6566272 | BLASTX | 607 | 4.00E−63 | 79 | (AB008016) similar to ribosomal protein S2 |
| | | | | | | | [Arabidopsis thaliana] |
| 2214 | LIB3602-087-Q6-K1-H2 | 4874265 | BLASTX | 595 | 2.00E−61 | 67 | (AC007354) EST gb\|F13926 comes from this gene. |
| | | | | | | | [Arabidopsis thaliana] |
| 2215 | LIB3602-059-Q6-K1-E6 | 2982297 | BLASTX | 584 | 3.00E−60 | 68 | (AF051233) KIAA0107-like protein [Picea |
| | | | | | | | mariana] |
| 2216 | LIB3602-054-Q6-K1-E11 | 7435895 | BLASTX | 145 | 6.00E−09 | 51 | multicatalytic endopeptidase complex (EC |
| | | | | | | | 3.4.99.46) beta chain - Arabidopsis thaliana |
| | | | | | | | >gi\|2511594\|emb\|CAA74028.1\| (Y13694) |
| | | | | | | | multicatalytic endopeptidase complex, proteasome |
| | | | | | | | precursor, beta subunit [Arabidopsis thaliana] |
| | | | | | | | >gi\|2827525\|emb\|CAA16533.1\| (AL02163 |
| 2217 | LIB3602-100-Q1-K1-G11 | 322577 | BLASTX | 294 | 3.00E−26 | 49 | Raf protein kinase homolog CTR1 - Arabidopsis |
| | | | | | | | thaliana |
| 2218 | LIB3602-040-Q6-K1-C4 | 7573420 | BLASTX | 414 | 1.00E−40 | 66 | (AL163816) translation releasing factor RF-1-like |
| | | | | | | | protein [Arabidopsis thaliana] |
| 2219 | LIB3602-055-Q6-K1-F7 | 2462761 | BLASTX | 434 | 9.00E−43 | 56 | (AC002292) Highly similar to auxin-induced |
| | | | | | | | protein (aldo/keto reductase family) [Arabidopsis |
| | | | | | | | thaliana] |
| 2220 | LIB3602-094-Q6-K6-E1 | 7294700 | BLASTX | 150 | 3.00E−09 | 59 | (AE003544) CG7351 gene product [Drosophila |
| | | | | | | | melanogaster] |
| 2221 | LIB3602-092-Q6-K6-F10 | 5281319 | BLASTX | 197 | 8.00E−15 | 34 | (AF134814) ceruloplasmin [Ovis aries] |
| 2222 | LIB3602-088-Q6-K6-F1 | 4587520 | BLASTX | 159 | 2.00E−10 | 45 | (AC007060) Strong similarity to gb\|X71057 protein |
| | | | | | | | kinase from Nicotiana tabacum and contains |
| | | | | | | | PF\|00069 eukaryotic protein kinase domain. |
| | | | | | | | [Arabidopsis thaliana] |
| 2223 | LIB3602-002-P1-K6-D10 | 3928758 | BLASTX | 520 | 1.00E−52 | 62 | (AB007987) Lipoic acid synthase [Arabidopsis |
| | | | | | | | thaliana] >gi\|4454462\|gb\|AAD20909.1\| |
| | | | | | | | (AC006234) lipoic acid synthase (LIP1) |
| | | | | | | | [Arabidopsis thaliana] |
| 2224 | LIB3602-049-Q6-K1-C11 | 2501356 | BLASTX | 406 | 1.00E−39 | 59 | TRANSKETOLASE, CHLOROPLAST |
| | | | | | | | PRECURSOR (TK) >gi\|1658322\|emb\|CAA90427.1\| |
| | | | | | | | (Z50099) transketolase precursor [Solanum |
| | | | | | | | tuberosum] |
| 2225 | LIB3602-104-Q1-K1-H1 | 2494112 | BLASTX | 667 | 6.00E−70 | 66 | (AC002376) Match to Arabidopsis ATHKCP |
| | | | | | | | (gb\|L40948). ESTs gb\|ATTS0764, gb\|R90646, |
| | | | | | | | gb\|AA389809, gb\|ATTS2615 come from this gene. |
| | | | | | | | [Arabidopsis thaliana] |
| | | | | | | | >gi\|3126868\|gb\|AAC15999.1\| (AF061570) |
| | | | | | | | potassium channel beta subunit homolog |
| | | | | | | | [Arabidopsis thaliana] |
| 2226 | LIB3602-038-Q6-K1-B12 | 6503290 | BLASTX | 479 | 5.00E−48 | 56 | (AC011713) Similar to gb\|U21855 CCR4- |
| | | | | | | | associated factor 1 (CAF1) from Mus musculus. |
| | | | | | | | ESTs gb\|AAA394972, gb\|AA585812 and |
| | | | | | | | gb\|H77015 come from this gene. [Arabidopsis |
| | | | | | | | thaliana] |
| 2227 | LIB3602-111-Q1-K1-H3 | 2760362 | BLASTX | 190 | 4.00E−14 | 37 | (AF016511) 15.9 kDa subunit of RNA polymerase |
| | | | | | | | II [Arabidopsis thaliana] |
| 2228 | LIB3602-004-Q1-K1-F12 | 3122387 | BLASTX | 816 | 2.00E−87 | 67 | WD-40 REPEAT PROTEIN MSI1 |
| | | | | | | | >gi\|2394229\|gb\|AAB70242.1\| (AF016846) WD-40 |
| | | | | | | | repeat protein [Arabidopsis thaliana] |
| 2229 | LIB3602-032-Q6-K1-B8 | 7076763 | BLASTX | 615 | 6.00E−64 | 72 | (AL138658) citrate synthase-like protein |
| | | | | | | | [Arabidopsis thaliana] |
| 2230 | LIB3602-058-Q6-K1-B2 | 5566579 | BLASTN | 35 | 8.00E−10 | 92 | Lepilemur dorsalis genomic fragment, RAPD with |
| | | | | | | | primer operon OPH4, clone A8b |
| 2231 | LIB3602-001-P1-K6-D3 | 4666287 | BLASTX | 251 | 2.00E−21 | 50 | (D85764) cytosolic monodehydroascorbate |
| | | | | | | | reductase [Oryza sativa] |
| 2232 | LIB3602-056-Q6-K1-C11 | 3980378 | BLASTX | 147 | 3.00E−09 | 46 | (AC004561) putative RNA-binding protein |
| | | | | | | | [Arabidopsis thaliana] |
| 2233 | LIB3602-037-Q6-K1-H12 | 3687239 | BLASTX | 491 | 2.00E−49 | 66 | (AC005169) putative clathrin assembly protein |
| | | | | | | | [Arabidopsis thaliana] |
| 2234 | LIB3602-040-Q6-K1-A6 | 5002553 | BLASTX | 239 | 6.00E−20 | 42 | (AF074603) NonF [Streptomyces griseus subsp. |
| | | | | | | | griseus] |
| 2235 | LIB3602-025-Q6-K1-D4 | 6403492 | BLASTX | 373 | 1.00E−35 | 43 | (AC010871) unknown protein [Arabidopsis |
| | | | | | | | thaliana] |
| 2236 | LIB3602-012-Q6-K1-D8 | 994736 | BLASTX | 145 | 3.00E−09 | 74 | (M18327) LacOPZ-alpha peptide from pUC9; |
| | | | | | | | putative [unidentified cloning vector] |
| | | | | | | | >gi\|994738\|gb\|AAA75563.1\| (M18328) LacOPZ- |
| | | | | | | | alpha peptide from pUC9; putative [Cloning vector |
| | | | | | | | pBGS9+] >gi\|994740\|gb\|AAA75565.1\| (M18329) |
| | | | | | | | LacOPZ-alpha peptide from pUC9; putative |
| 2237 | LIB3602-006-Q1-K1-D11 | 6690272 | BLASTX | 216 | 5.00E−17 | 50 | (AF114386) putative protease HhoA precursor |
| | | | | | | | [Arabidopsis thaliana] |
| 2238 | LIB3602-023-Q6-K1-A2 | 4507277 | BLASTX | 289 | 9.00E−26 | 38 | serine/threonine kinase 2 |
| | | | | | | | >gi\|1709347\|sp\|P51957\|STK2_HUMAN |
| | | | | | | | SERINE/THREONINE-PROTEIN KINASE NRK2 |
| | | | | | | | (SERINE/THREONINE KINASE 2) |
| | | | | | | | >gi\|7427997\|pir\|\|I78885 serine/threonine-specific |
| | | | | | | | protein kinase (EC 2.7.1.—) STK2 - human |
| | | | | | | | >gi\|348245\|gb\|AAA36658.1\| (L20321) prote |
| 2239 | LIB3602-052-Q6-K1-D6 | 7471680 | BLASTX | 153 | 7.00E−10 | 37 | conserved hypothetical protein - Deinococcus |
| | | | | | | | radiodurans (strain R1) |
| | | | | | | | >gi\|6458157\|gb\|AAF10050.1\|AE001906_6 |
| | | | | | | | (AE001906) conserved hypothetical protein |
| | | | | | | | [Deinococcus radiodurans] |
| 2240 | LIB3602-019-Q6-K1-H3 | 3201626 | BLASTX | 143 | 5.00E−09 | 37 | (AC004669) putative protein kinase [Arabidopsis |
| | | | | | | | thaliana] |
| 2241 | LIB3602-022-Q6-K1-G3 | 6970044 | BLASTX | 481 | 3.00E−48 | 61 | (AF195243) apospory-associated protein C; APOC |
| | | | | | | | [Chlamydomonas reinhardtii] |
| 2242 | LIB3602-066-Q1-K6-F9 | 7208213 | BLASTX | 147 | 4.00E−09 | 41 | (AF160800) constitutive photomorphogenic 11 |
| | | | | | | | [Oryza sativa subsp. indica] |
| 2243 | LIB3602-045-Q6-K1-B1 | 7241508 | BLASTN | 43 | 4.00E−15 | 88 | Sequence 36 from Patent EP0892047 |
| 2244 | LIB3602-103-Q1-K1-E6 | 2462752 | BLASTX | 345 | 9.00E−41 | 56 | (AC002292) Phosphatidylinositol 3-kinase |
| | | | | | | | [Arabidopsis thaliana] |
| 2245 | LIB3602-085-Q6-K1-D8 | 2493046 | BLASTX | 193 | 2.00E−14 | 55 | ATP SYNTHASE DELTA′ CHAIN, |
| | | | | | | | MITOCHONDRIAL PRECURSOR |
| | | | | | | | >gi\|82297\|pir\|\|A41740 H+-transporting ATP |
| | | | | | | | synthase (EC 3.6.1.34) delta′ chain precursor - sweet |
| | | | | | | | potato >gi\|217938\|dbj\|BAA01511.1\| (D10660) |
| | | | | | | | mitochondrial F1-ATPase delta subunit [Ipomoea |
| | | | | | | | batatas] |
| 2246 | LIB3602-079-Q6-K6-C5 | 7303555 | BLASTX | 338 | 2.00E−31 | 48 | (AE003824) CG8983 gene product [Drosophila |
| | | | | | | | melanogaster] |
| 2247 | LIB3602-013-Q6-K1-G3 | 484466 | BLASTX | 345 | 3.00E−32 | 41 | glucan endo-1,3-beta-D-glucosidase (EC 3.2.1.39) |
| | | | | | | | precursor - Bacillus circulans (strain IAM1165) |
| | | | | | | | >gi\|452882\|gb\|AAC60453.1\| (S67033) beta-1,3- |
| | | | | | | | glucanase {EC 3.2.1.39} [Bacillus circulans, |
| | | | | | | | IAM1165, Peptide, 877 aa] |
| | | | | | | | >gi\|601878\|dbj\|BAA04469.1\| (D17519) beta-1, |
| 2248 | LIB3602-017-Q6-K1-A8 | 7362989 | BLASTN | 34 | 2.00E−09 | 82 | Homo sapiens partial mRNA, clone c4-1b12 |
| 2249 | LIB3602-035-Q1-K1-A7 | 2500295 | BLASTX | 275 | 6.00E−24 | 50 | 50S RIBOSOMAL PROTEIN L21 |
| | | | | | | | >gi\|7428682\|pir\|\|S75901 ribosomal protein L21 - |
| | | | | | | | Synechocystis sp. (strain PCC 6803) |
| | | | | | | | >gi\|1653446\|dbj\|BAA18360.1\| (D90913) 50S |
| | | | | | | | ribosomal protein L21 [Synechocystis sp.] |
| 2250 | LIB3602-049-Q6-K1-A4 | 2501188 | BLASTX | 194 | 3.00E−15 | 71 | THIAZOLE BIOSYNTHETIC ENZYME |
| | | | | | | | PRECURSOR (ARA6) >gi\|2129750\|pir\|\|S71191 |
| | | | | | | | thiamin biosynthesis protein thi4 - Arabidopsis |
| | | | | | | | thaliana >gi\|1113783\|gb\|AAC97124.1\| (U17589) |
| | | | | | | | Thi1 protein [Arabidopsis thaliana] |
| 2251 | LIB3602-018-Q6-K1-D11 | 400989 | BLASTX | 150 | 9.00E−10 | 56 | 50S RIBOSOMAL PROTEIN L24, |
| | | | | | | | CHLOROPLAST PRECURSOR (CL24) |
| | | | | | | | >gi\|322771\|pir\|\|A45113 ribosomal protein L24 |
| | | | | | | | precursor - common tobacco |
| | | | | | | | >gi\|170273\|gb\|AAA34086.1\| (M87838) ribosomal |
| | | | | | | | protein L24 [Nicotiana tabacum] |
| | | | | | | | >gi\|170324\|gb\|AAA34114.1\| (M87839) ribosomal |
| | | | | | | | prote |
| 2252 | LIB3602-086-Q6-K1-E1 | 6598752 | BLASTX | 300 | 6.00E−27 | 64 | (AC007290) putative chromosome associated |
| | | | | | | | protein [Arabidopsis thaliana] |
| 2253 | LIB3602-059-Q6-K1-E8 | 7434997 | BLASTX | 268 | 3.00E−23 | 32 | lysosomal acid lipase - human |
| | | | | | | | >gi\|505053\|gb\|AAB60328.1\| (U08464) lysosomal |
| | | | | | | | acid lipase [Homo sapiens] |
| | | | | | | | >gi\|506431\|emb\|CAA83495.1\| (Z31690) lysosomal |
| | | | | | | | acid lipase [Homo sapiens] |
| 2254 | LIB3602-026-Q6-K1-H10 | 7462772 | BLASTX | 170 | 9.00E−12 | 30 | hypothetical protein TM0484 - Thermotoga |
| | | | | | | | maritima (strain MSB8) |
| | | | | | | | >gi\|4980995\|gb\|AAD35569.1\|AE001726_3 |
| | | | | | | | (AE001726) pyrimidine precursor biosynthesis |
| | | | | | | | enzyme, putative [Thermotoga maritima] |
| 2255 | LIB3602-042-Q6-K1-B1 | 2443886 | BLASTX | 363 | 2.00E−34 | 64 | (AC002294) Unknown protein [Arabidopsis |
| | | | | | | | thaliana] |
| 2256 | LIB3602-028-Q6-K1-D7 | 133891 | BLASTX | 222 | 7.00E−18 | 50 | 40S RIBOSOMAL PROTEIN S26 (DS31) |
| | | | | | | | >gi\|71054\|pir\|\|R3FF26 ribosomal protein S26 - fruit |
| | | | | | | | fly (Drosophila melanogaster) |
| | | | | | | | >gi\|8494\|emb\|CAA32463.1\| (X14247) ribosomal |
| | | | | | | | protein S31 [Drosophila melanogaster] |
| | | | | | | | >gi\|4469169\|emb\|CAB38441.1\| (X13625) |
| | | | | | | | ribosomal protein S31 |
| 2257 | LIB3602-119-Q1-K1-B6 | 2500243 | BLASTX | 151 | 3.00E−10 | 72 | 50S RIBOSOMAL PROTEIN L6 |
| | | | | | | | >gi\|2127652\|pir\|\|S62827 ribosomal protein L6 - |
| | | | | | | | Mycoplasma pneumoniae (strain ATCC 29342) |
| | | | | | | | >gi\|2146617\|pir\|\|S73977 ribosomal protein L6 - |
| | | | | | | | Mycoplasma pneumoniae (SGC3) (ATCC 29342) |
| | | | | | | | >gi\|1215708\|gb\|AAC43701.1\| (U34795) RplF |
| | | | | | | | [Mycoplasma |
| 2258 | LIB3602-074-Q1-K1-D5 | 476608 | BLASTN | 37 | 4.00E−11 | 91 | Drosophila melanogaster ribosomal protein L19 |
| | | | | | | | mRNA |
| 2259 | LIB3602-032-Q6-K1-C3 | 7572912 | BLASTX | 150 | 2.00E−09 | 38 | (AL163763) putative protein [Arabidopsis thaliana] |
| 2260 | LIB3602-048-Q6-K1QA-E2 | 1707018 | BLASTX | 287 | 2.00E−25 | 52 | (U78721) putative related to microbial divalent |
| | | | | | | | cation tolerance proteins [Arabidopsis thaliana] |
| 2261 | LIB3602-059-Q6-K1-A2 | 6630731 | BLASTX | 425 | 1.00E−41 | 45 | (AL132958) RNA helicase-like protein |
| | | | | | | | [Arabidopsis thaliana] |
| 2262 | LIB3602-074-Q1-K1-A9 | 1703457 | BLASTX | 288 | 2.00E−25 | 51 | CATION-TRANSPORTING ATPASE CA1 |
| | | | | | | | >gi\|1103646\|emb\|CAA63790.1\| (X93592) CA1 |
| | | | | | | | [Dunaliella bioculata] |
| 2263 | LIB3602-085-Q6-K1-E4 | 6630733 | BLASTX | 510 | 1.00E−64 | 71 | (AL132958) Cytochrom P450-like protein |
| | | | | | | | [Arabidopsis thaliana] |
| 2264 | LIB3602-119-Q1-K1-H10 | 1350947 | BLASTX | 156 | 1.00E−11 | 78 | 40S RIBOSOMAL PROTEIN S18 |
| | | | | | | | >gi\|1076216\|pir\|\|S51145 ribosomal protein S18.e, |
| | | | | | | | cytosolic - Chlamydomonas reinhardtii |
| | | | | | | | >gi\|624957\|emb\|CAA58668.1\| (X83693) ribosomal |
| | | | | | | | protein S18 [Chlamydomonas reinhardtii] |
| | | | | | | | >gi\|1587084\|prf\|\|2205351A ribosomal protein S18 |
| | | | | | | | [Chlamyd |
| 2265 | LIB3602-103-Q1-K1-E1 | 7209723 | BLASTX | 244 | 2.00E−20 | 43 | (AB034912) WD-repeat like sequence [Homo |
| | | | | | | | sapiens] |
| 2266 | LIB3602-014-Q6-K1-A2 | 2511590 | BLASTX | 301 | 2.00E−27 | 58 | (Y13692) multicatalytic endopeptidase complex, |
| | | | | | | | proteasome component, beta subunit [Arabidopsis |
| | | | | | | | thaliana] >gi\|3421111\|gb\|AAC32070.1\| (AF043534) |
| | | | | | | | 20S proteasome beta subunit PBD1 [Arabidopsis |
| | | | | | | | thaliana] |
| 2267 | LIB3602-007-Q1-K1-H12 | 7488326 | BLASTX | 315 | 1.00E−28 | 56 | SEN1 protein homolog T20K24.14 - Arabidopsis |
| | | | | | | | thaliana >gi\|3176714\|gb\|AAD12029.1\| (AC002392) |
| | | | | | | | putative DNA2-NAM7 helicase family protein |
| | | | | | | | [Arabidopsis thaliana] |
| 2268 | LIB3602-039-Q6-K1-D5 | 7485815 | BLASTX | 279 | 1.00E−24 | 60 | hypothetical protein F19I3.9 - Arabidopsis thaliana |
| | | | | | | | >gi\|3033382\|gb\|AAC12826.1\| (AC004238) |
| | | | | | | | unknown protein [Arabidopsis thaliana] |
| 2269 | LIB3602-035-Q1-K1-E5 | 7297653 | BLASIX | 232 | 4.00E−19 | 40 | (AE003628) CG4968 gene product [Drosophila |
| | | | | | | | melanogaster] |
| 2270 | LIB3602-118-Q1-K1-H9 | 7484927 | BLASTX | 146 | 6.00E−09 | 57 | DNA-3-methyladenine glycosidase I homolog |
| | | | | | | | F18B3.160 - Arabidopsis thaliana |
| | | | | | | | >gi\|4835239\|emb\|CAB42917.1\| (AL049862) |
| | | | | | | | putative DNA-3-methyladenine glycosidase |
| | | | | | | | [Arabidopsis thaliana] |
| 2271 | LIB3602-039-Q6-K1-B2 | 5669636 | BLASTX | 328 | 3.00E−30 | 44 | (AF096247) ethylene-responsive elongation factor |
| | | | | | | | EF-Ts precursor [Lycopersicon esculentum] |
| 2272 | LIB3602-077-Q6-K6-G10 | 5669638 | BLASTX | 417 | 1.00E−40 | 74 | (AF096248) ethylene-responsive RNA helicase |
| | | | | | | | [Lycopersicon esculentum] |
| 2273 | LIB3602-039-Q6-K1-E12 | 7459497 | BLASTX | 194 | 1.00E−14 | 32 | hypothetical protein sll0886 - Synechocystis sp. |
| | | | | | | | (strain PCC 6803) >gi\|1001204\|dbj\|BAA10444.1\| |
| | | | | | | | (D64003) hypothetical protein [Synechocystis sp.] |
| 2274 | LIB3602-012-Q6-K1-C11 | 404015 | BLASTX | 174 | 9.00E−24 | 59 | (U02032) ribosomal protein L23a [Homo sapiens] |
| 2275 | LIB3602-094-Q6-K6-F8 | 1172599 | BLASTX | 354 | 3.00E−33 | 54 | PROTEASOME COMPONENT C5 |
| | | | | | | | (MULTICATALYTIC ENDOPEPTIDASE |
| | | | | | | | COMPLEX SUBUNIT C5) (TAS-F22/FAFP98) |
| | | | | | | | >gi\|600387\|emb\|CAA47753.1\| (X67338) |
| | | | | | | | proteosome subunit [Arabidopsis thaliana] |
| 2276 | LIB3602-084-Q6-K1-H1 | 1942873 | BLASTX | 210 | 2.00E−16 | 47 | Thioredoxin H (Oxidized Form), Nmr, 23 Structures |
| 2277 | LIB3602-061-Q6-K1-E4 | 7512679 | BLASTX | 484 | 1.00E−48 | 68 | hypothetical protein DKFZp434N241.1 - human |
| | | | | | | | (fragment) >gi\|5911980\|emb\|CAB55960.1\| |
| | | | | | | | (AL117492) hypothetical protein [Homo sapiens] |
| 2278 | LIB3602-040-Q6-K1-E7 | 7488346 | BLASTX | 316 | 6.00E−29 | 62 | spliceosome-associated protein SAP62 homolog |
| | | | | | | | T26B15.16 - Arabidopsis thaliana |
| | | | | | | | >gi\|3298548\|gb\|AAC25942.1\| (AC004681) putative |
| | | | | | | | spliceosome-associated protein [Arabidopsis |
| | | | | | | | thaliana] |
| 2279 | LIB3602-077-Q6-K6-H12 | 7484782 | BLASTX | 286 | 3.00E−25 | 42 | 4-alpha-glucanotransferase homolog T20B5.4 - |
| | | | | | | | Arabidopsis thaliana >gi\|2623298\|gb\|AAB86444.1\| |
| | | | | | | | (AC002409) 4-alpha-glucanotransferase |
| | | | | | | | [Arabidopsis thaliana] |
| 2280 | LIB3602-034-Q6-K1-E4 | 732535 | BLASTN | 39 | 5.00E−13 | 90 | Volvox carteri oxygen-evolving enhancer protein 3 |
| | | | | | | | mRNA, complete cds |
| 2281 | LIB3602-108-Q1-K1-D1 | 7484784 | BLASTX | 219 | 1.00E−17 | 33 | ABC-type transport protein homolog F12C20.5 - |
| | | | | | | | Arabidopsis thaliana >gi\|3426037\|gb\|AAC32236.1\| |
| | | | | | | | (AC005168) putative ABC transporter [Arabidopsis |
| | | | | | | | thaliana] |
| 2282 | LIB3602-111-Q1-K1-B8 | 3043694 | BLASTX | 182 | 3.00E−23 | 38 | (AB011157) KIAA0585 protein [Homo sapiens] |
| 2283 | LIB3602-109-Q1-K1-D4 | 2492772 | BLASTX | 178 | 9.00E−23 | 73 | ALCOHOL DEHYDROGENASE CLASS III |
| | | | | | | | (GLUTATHIONE-DEPENDENT |
| | | | | | | | FORMALDEHYDE DEHYDROGENASE) (FDH) |
| | | | | | | | (FALDH) (GSH-FDH) |
| | | | | | | | >gi\|1498024\|gb\|AAB06322.1\| (U63931) |
| | | | | | | | glutathione-dependent formaldehyde dehydrogenase |
| | | | | | | | [Arabidopsis thaliana] |
| 2284 | LIB3602-039-Q6-K1-F2 | 7431747 | BLASTX | 176 | 2.00E−12 | 52 | probable acyl-CoA dehydrogenase - Mycobacterium |
| | | | | | | | tuberculosis (strain H37RV) |
| | | | | | | | >gi\|2911026\|emb\|CAA17519.1\| (AL021958) fadE9 |
| | | | | | | | [Mycobacterium tuberculosis] |
| 2285 | LIB3602-085-Q6-K1-F10 | 7485823 | BLASTX | 162 | 9.00E−11 | 45 | hypothetical protein F1C12.70 - Arabidopsis |
| | | | | | | | thaliana >gi\|2982432\|emb\|CAA18240.1\| |
| | | | | | | | (AL022224) putative protein [Arabidopsis thaliana] |
| | | | | | | | >gi\|7268810\|emb\|CAB79015.1\| (AL161552) |
| | | | | | | | putative protein [Arabidopsis thaliana] |
| 2286 | LIB3602-041-Q6-K1-A10 | 7459661 | BLASTX | 242 | 4.00E−20 | 42 | transcription initiation factor IIA gamma chain |
| | | | | | | | homolog T22A6.270 - Arabidopsis thaliana |
| | | | | | | | >gi\|2826882\|emb\|CAA11524.1\| (AJ223634) |
| | | | | | | | transcription factor IIA small subunit [Arabidopsis |
| | | | | | | | thaliana] >gi\|5051786\|emb\|CAB45079.1\| |
| | | | | | | | (AL078637) transcription factor IIA s |
| 2287 | LIB3602-020-Q6-K1-F8 | 4689382 | BLASTX | 315 | 7.00E−29 | 57 | (AF139466) chlorophyll a/b binding protein CP29 |
| | | | | | | | [Vigna radiata] |
| 2288 | LIB3602-048-Q6-K1QA-C6 | 2811131 | BLASTX | 181 | 5.00E−13 | 66 | (AF013216) isocitrate lyase [Myxococcus xanthus] |
| 2289 | LIB3602-111-Q1-K1-D11 | 5103845 | BLASTX | 279 | 2.00E−32 | 49 | (AC007591) Strong similarity to gb\|AF096285 |
| | | | | | | | serine-threonine kinase receptor-associated protein |
| | | | | | | | from Mus musculus and contains 5 PF\|00400 |
| | | | | | | | WD40, G-beta repeat domains. EST gb\|F14050 |
| | | | | | | | comes from this gene. [Arabidopsis thaliana] |
| 2290 | LIB3602-086-Q6-K1-B5 | 7487272 | BLASTX | 171 | 8.00E−12 | 32 | hypothetical protein T20K18.240 - Arabidopsis |
| | | | | | | | thaliana >gi\|4586265\|emb\|CAB41006.1\| |
| | | | | | | | (AL049640) putative protein [Arabidopsis thaliana] |
| | | | | | | | >gi\|7267991\|emb\|CAB78331.1\| (AL161535) |
| | | | | | | | putative protein [Arabidopsis thaliana] |
| 2291 | LIB3602-001-P1-K6-C3 | 3746062 | BLASTX | 150 | 2.00E−09 | 45 | (AC005311) hypothetical protein [Arabidopsis |
| | | | | | | | thaliana] |
| 2292 | LIB3602-084-Q6-K1-H3 | 7452056 | BLASTX | 160 | 1.00E−10 | 67 | hypothetical protein T29H11.100 - Arabidopsis |
| | | | | | | | thaliana >gi\|4678350\|emb\|CAB41160.1\| |
| | | | | | | | (AL049659) putative protein [Arabidopsis thaliana] |
| 2293 | LIB3602-088-Q6-K6-H4 | 6166507 | BLASTX | 168 | 8.00E−12 | 56 | (AF198092) RP42 [Mus musculus] |
| 2294 | LIB3602-036-Q6-K1-D3 | 7406456 | BLASTX | 256 | 9.00E−22 | 38 | (AL162875) 3-oxoacyl-[acyl-carrier-protein] |
| | | | | | | | synthase-like protein [Arabidopsis thaliana] |
| 2295 | LIB3602-092-Q6-K6-C8 | 131196 | BLASTX | 499 | 3.00E−50 | 69 | PHOTOSYSTEM I REACTION CENTRE |
| | | | | | | | SUBUNIT VI PRECURSOR (LIGHT- |
| | | | | | | | HARVESTING COMPLEX I 11 KD PROTEIN) |
| | | | | | | | (PSI-H) >gi\|82412\|pir\|\|S05012 photosystem I |
| | | | | | | | protein psaH precursor - barley |
| | | | | | | | >gi\|19093\|emb\|CAA34218.1\| (X16092) 10.2 kDa |
| | | | | | | | photosystem I polypeptide [Hordeum vulga |
| 2296 | LIB3602-032-Q6-K1-A7 | 7484752 | BLASTX | 355 | 2.00E−33 | 54 | L-ascorbate peroxidase (EC 1.11.1.11), cytosolic - |
| | | | | | | | cucumber >gi\|1669585\|dbj\|BAA13671.1\| (D88649) |
| | | | | | | | cytosolic ascorbate peroxidase [Cucumis sativus] |
| 2297 | LIB3602-019-Q6-K1-G8 | 6714305 | BLASTX | 183 | 2.00E−13 | 38 | (AC013354) F15H18.4 [Arabidopsis thaliana] |
| 2298 | LIB3602-053-Q6-K1-H10 | 7379607 | BLASTX | 381 | 2.00E−36 | 59 | (AL162754) putative |
| | | | | | | | amidophosphoribosyltransferase [Neisseria |
| | | | | | | | meningitidis] |
| 2299 | LIB3602-078-Q6-K6-C4 | 267073 | BLASTX | 669 | 4.00E−72 | 93 | TUBULIN BETA-2/BETA-3 CHAIN |
| | | | | | | | >gi\|320184\|pir\|\|JQ1587 tubulin beta chain - |
| | | | | | | | Arabidopsis thaliana >gi\|166898\|gb\|AAA32881.1\| |
| | | | | | | | (M84700) beta-2 tubulin [Arabidopsis thaliana] |
| | | | | | | | >gi\|166900\|gb\|AAA32882.1\| (M84701) beta-3 |
| | | | | | | | tubulin [Arabidopsis thaliana] |
| 2300 | LIB3602-003-Q1-K1-F11 | 4885539 | BLASTX | 234 | 3.00E−19 | 51 | protein-L-isoaspartate (D-aspartate) O- |
| | | | | | | | methyltransferase >gi\|1332399\|dbj\|BAA05028.1\| |
| | | | | | | | (D25545) PIMT isozyme I [Homo sapiens] |
| | | | | | | | >gi\|1332403\|dbj\|BAA05030.1\| (D25547) PIMT |
| | | | | | | | isozyme I [Homo sapiens] |
| 2301 | LIB3602-078-Q6-K6-G4 | 4929769 | BLASTX | 170 | 9.00E−12 | 68 | (AF151908) CGI-150 protein [Homo sapiens] |
| 2302 | LIB3602-021-Q6-K1-A2 | 1710560 | BLASTX | 166 | 9.00E−12 | 93 | 60S RIBOSOMAL PROTEIN L40 (CEP52) |
| 2303 | LIB3602-006-Q1-K1-D6 | 5107214 | BLASTX | 196 | 1.00E−14 | 63 | (AF150113) small zinc finger-like protein [Oryza |
| | | | | | | | sativa] |
| 2304 | LIB3602-009-Q6-K1-D6 | 549986 | BLASTX | 228 | 1.00E−18 | 46 | (U13149) possible apospory-associated protein |
| | | | | | | | [Pennisetum ciliare] |
| 2305 | LIB3602-101-Q1-K1-E8 | 7486379 | BLASTX | 376 | 5.00E−36 | 57 | hypothetical protein F3L17.100 - Arabidopsis |
| | | | | | | | thaliana >gi\|5262764\|emb\|CAB45912.1\| |
| | | | | | | | (AL080283) putative protein [Arabidopsis thaliana] |
| | | | | | | | >gi\|7270056\|emb\|CAB79871.1\| (AL161579) |
| | | | | | | | putative protein [Arabidopsis thaliana] |
| 2306 | LIB3602-118-Q1-K1-E11 | 3617770 | BLASTX | 358 | 1.00E−33 | 56 | (Y14329) threonyl-tRNA synthetase [Arabidopsis |
| | | | | | | | thaliana] |
| 2307 | LIB3602-111-Q1-K1-G9 | 1351669 | BLASTX | 206 | 5.00E−16 | 36 | HYPOTHETICAL 44.3 KD PROTEIN C1F7.07C |
| | | | | | | | IN CHROMOSOME I >gi\|2130298\|pir\|\|S62579 |
| | | | | | | | hypothetical protein SPAC1F7.07c - fission yeast |
| | | | | | | | (Schizosaccharomyces pombe) |
| | | | | | | | >gi\|7492832\|pir\|\|T38101 probable plasma |
| | | | | | | | membrane iron permease - fission yeast |
| | | | | | | | (Schizosaccharomyces |
| 2308 | LIB3602-046-Q6-K1-E8 | 1350778 | BLASTX | 547 | 5.00E−56 | 66 | 60S ACIDIC RIBOSOMAL PROTEIN P0 (L10E) |
| | | | | | | | >gi\|2119118\|pir\|\|I50151 acidic ribosomal |
| | | | | | | | phosphoprotein - chicken |
| | | | | | | | >gi\|453474\|gb\|AAC38020.1\| (L28704) acidic |
| | | | | | | | ribosomal phosphoprotein [Gallus gallus] |
| 2309 | LIB3602-048-Q6-K1QA-G3 | 6470348 | BLASTX | 305 | 1.00E−27 | 52 | (AF002248) PSI light-harvesting antenna |
| | | | | | | | chlorophyll a/b-binding protein [Pisum sativum] |
| 2310 | LIB3602-092-Q6-K6-A11 | 4574139 | BLASTX | 545 | 8.00E−57 | 58 | (AF073697) cysteine synthase [Oryza sativa] |
| 2311 | LIB3602-015-Q6-K1-B8 | 7431577 | BLASTX | 537 | 9.00E−55 | 62 | probable pyruvate dehydrogenase (lipoamide) (EC |
| | | | | | | | 1.2.4.1) E1 beta chain - Synechocystis sp. (strain |
| | | | | | | | PCC 6803) >gi\|1652524\|dbj\|BAA17445.1\| |
| | | | | | | | (D90906) pyruvate dehydrogenase E1 beta subunit |
| | | | | | | | [Synechocystis sp.] |
| 2312 | LIB3602-041-Q6-K1-C8 | 7469613 | BLASTX | 264 | 1.00E−22 | 61 | hypothetical protein - Synechocystis sp. (strain PCC |
| | | | | | | | 6803) >gi\|1001629\|dbj\|BAA10360.1\| (D64002) |
| | | | | | | | hypothetical protein [Synechocystis sp.] |
| 2313 | LIB3602-092-Q6-K6-E2 | 2493650 | BLASTX | 268 | 4.00E−23 | 79 | RUBISCO SUBUNIT BINDING-PROTEIN BETA |
| | | | | | | | SUBUNIT (60 KD CHAPERONIN BETA |
| | | | | | | | SUBUNIT) (CPN-60 BETA) |
| | | | | | | | >gi\|1167858\|emb\|CAA93139.1\| (Z68903) |
| | | | | | | | chaperonin [Secale cereale] |
| 2314 | LIB3602-055-Q6-K1-H11 | 21309 | BLASTX | 150 | 8.00E−10 | 45 | (X57955) 28 kD RNA binding protein [Spinacia |
| | | | | | | | oleracea] |
| 2315 | LIB3602-005-Q1-K1-D7 | 5103820 | BLASTX | 359 | 8.00E−34 | 34 | (AC007591) Similar to gb\|Z70524 PDR5-like ABC |
| | | | | | | | transporter from Spirodela polyrrhiza and is a |
| | | | | | | | member of the PF\|00005 ABC transporter family. |
| | | | | | | | ESTs gb\|N97039 and gb\|T43169 come from this |
| | | | | | | | gene. [Arabidopsis thaliana] |
| 2316 | LIB3602-115-Q1-K1-B2 | 586845 | BLASTX | 235 | 3.00E−19 | 42 | PROBABLE MALTOSE O- |
| | | | | | | | ACETYLTRANSFERASE (MALTOSE |
| | | | | | | | TRANSACETYLASE) >gi\|2126884\|pir\|\|I39920 |
| | | | | | | | probable O-acetyltransferase (EC 2.3.1.—) yyal - |
| | | | | | | | Bacillus subtilis >gi\|438465\|gb\|AAA64343.1\| |
| | | | | | | | (L16865) Probable operon with orfF. Possible |
| | | | | | | | alternative initiation codon, b |
| 2317 | LIB3602-037-Q6-K1-E3 | 1350930 | BLASTX | 513 | 6.00E−52 | 84 | 40S RIBOSOMAL PROTEIN S13 |
| 2318 | LIB3602-060-Q6-K1-F6 | 4914332 | BLASTX | 164 | 4.00E−11 | 57 | (AC005489) F14N23.18 [Arabidopsis thaliana] |
| 2319 | LIB3602-030-Q6-K1-G8 | 6093612 | BLASTX | 563 | 6.00E−58 | 71 | PYRUVATE DEHYDROGENASE E1 |
| | | | | | | | COMPONENT BETA SUBUNIT, |
| | | | | | | | MITOCHONDRIAL PRECURSOR (PDHE1-B) |
| | | | | | | | >gi\|520478\|gb\|AAA52225.1\| (U09137) pyruvate |
| | | | | | | | dehydrogenase E1 beta subunit [Arabidopsis |
| | | | | | | | thaliana] >gi\|1090498\|prf\|\|2019230A pyruvate |
| | | | | | | | dehydrogenase [Arabidopsis thaliana] |
| 2320 | LIB3602-074-Q1-K1-F6 | 5911363 | BLASTX | 210 | 2.00E−16 | 50 | (AF159699) putative host factor [Nicotiana |
| | | | | | | | tabacum] |
| 2321 | LIB3602-103-Q1-K1-H10 | 7469637 | BLASTX | 145 | 1.00E−16 | 62 | hypothetical protein - Synechocystis sp. (strain PCC |
| | | | | | | | 6803) >gi\|1001676\|dbj\|BAA10411.1\| (D64002) |
| | | | | | | | hypothetical protein [Synechocystis sp.] |
| 2322 | LIB3602-102-Q1-K1-H8 | 3327210 | BLASTX | 161 | 1.00E−10 | 30 | (AB014598) KIAA0698 protein [Homo sapiens] |
| 2323 | LIB3602-107-Q1-K1-H2 | 7544040 | BLASTX | 696 | 2.00E−73 | 67 | (AL163641) acetyl/propionyl CoA carboxylase, |
| | | | | | | | beta subunit [Streptomyces coelicolor A3(2)] |
| 2324 | LIB3602-088-Q6-K6-E12 | 7428213 | BLASTX | 233 | 5.00E−19 | 45 | endopeptidase Clp ATP-binding chain C - |
| | | | | | | | Synechococcus sp. (strain PCC 7942) |
| | | | | | | | >gi\|755163\|gb\|AAB67745.1\| (U16134) ClpC |
| | | | | | | | [Synechococcus sp.] |
| 2325 | LIB3602-026-Q6-K1-H11 | 81679 | BLASTX | 199 | 4.00E−15 | 61 | acyl carrier protein precursor (clone 34C02) - rape |
| 2326 | LIB3602-048-Q6-K1QA- | 6730727 | BLASTX | 492 | 1.00E−49 | 60 | (AC018849) putative acetylornithine transaminase |
| | B11 | | | | | | [Arabidopsis thaliana] |
| 2327 | LIB3602-055-Q6-K1-H9 | 3913791 | BLASTX | 269 | 2.00E−23 | 48 | GLUTAMATE--CYSTEINE LIGASE |
| | | | | | | | PRECURSOR (GAMMA-GLUTAMYLCYSTEINE |
| | | | | | | | SYNTHETASE) (GAMMA-ECS) (GCS) |
| | | | | | | | >gi\|7489004\|pir\|\|T04332 glutamate-cysteine ligase |
| | | | | | | | (EC 6.3.2.2) - tomato >gi\|2407615\|gb\|AAB71230.1\| |
| | | | | | | | (AF017983) gamma-glutamylcysteine synthetase |
| | | | | | | | [Lycopersicon escul |
| 2328 | LIB3602-005-Q1-K1-H11 | 266567 | BLASTX | 329 | 3.00E−30 | 48 | MITOCHONDRIAL PROCESSING PEPTIDASE |
| | | | | | | | ALPHA SUBUNIT PRECURSOR (ALPHA-MPP) |
| | | | | | | | (UBIQUINOL-CYTOCHROME C REDUCTASE |
| | | | | | | | SUBUNIT II) >gi\|421956\|pir\|\|S23558 mitochondrial |
| | | | | | | | processing peptidase (EC 3.4.99.41) alpha chain |
| | | | | | | | precursor - potato >gi\|21493\|emb\|CAA46990.1\| |
| | | | | | | | (X66284) |
| 2329 | LIB3602-063-Q1-K6-G10 | 6984214 | BLASTX | 481 | 2.00E−68 | 62 | (AF228639) lipoamide dehydrogenase precursor |
| | | | | | | | [Arabidopsis thaliana] |
| 2330 | LIB3602-087-Q6-K1-F7 | 7499051 | BLASTX | 328 | 4.00E−47 | 62 | hypothetical protein F13H8.7 - Caenorhabditis |
| | | | | | | | elegans >gi\|722377\|gb\|AAC46683.1\| (U23139) |
| | | | | | | | highly similar to beta-ureidopropionase |
| | | | | | | | (SP: BUP_RAT) [Caenorhabditis elegans] |
| 2331 | LIB3602-028-Q6-K1-H12 | 7488521 | BLASTX | 163 | 6.00E−11 | 43 | cysteine proteinase inhibitor BCPI-2-turnip |
| | | | | | | | >gi\|1256424\|gb\|AAA96316.1\| (U51119) cysteine |
| | | | | | | | proteinase inhibitor [Brassica rapa] |
| 2332 | LIB3602-076-Q6-K6-C3 | 6984216 | BLASTX | 238 | 6.00E−29 | 52 | (AF228640) lipoamide dehydrogenase precursor |
| | | | | | | | [Arabidopsis thaliana] |
| 2333 | LIB3602-076-Q6-K6-H12 | 4884860 | BLASTX | 157 | 8.00E−11 | 60 | (AF133118) nucleic acid binding protein [Oryza |
| | | | | | | | sativa] |
| 2334 | LIB3602-050-Q6-K1-E12 | 7488367 | BLASTX | 203 | 1.00E−15 | 37 | thiamin pyrophosphokinase homolog F16B22.24 - |
| | | | | | | | Arabidopsis thaliana >gi\|3341695\|gb\|AAC27477.1\| |
| | | | | | | | (AC003672) putative thiamin pyrophosphokinase |
| | | | | | | | [Arabidopsis thaliana] |
| 2335 | LIB3602-101-Q1-K1-A3 | 7106101 | BLASTX | 358 | 2.00E−42 | 67 | (AL157917) ap-1 golgi-associated clathrin assembly |
| | | | | | | | protein complex component [Schizosaccharomyces |
| | | | | | | | pombe] |
| 2336 | LIB3602-111-Q1-K1-E7 | 1749716 | BLASTX | 359 | 6.00E−34 | 63 | (D89254) similar to Saccharomyces cerevisiae |
| | | | | | | | dihydroxy-acid dehydratase precursor, SWISS- |
| | | | | | | | PROT Accession Number P39522 |
| | | | | | | | [Schizosaccharomyces pombe] |
| 2337 | LIB3602-073-Q1-K1-G6 | 2398531 | BLASTX | 263 | 1.00E−22 | 70 | (Y13726) Transcription factor [Arabidopsis |
| | | | | | | | thaliana] |
| 2338 | LIB3602-088-Q6-K6-A3 | 7077174 | BLASTX | 163 | 2.00E−11 | 60 | (AB035256) eukaryotic polypeptide chain release |
| | | | | | | | factor 3 [Oryctolagus cuniculus] |
| 2339 | LIB3602-050-Q6-K1-H2 | 7290447 | BLASTX | 166 | 2.00E−11 | 49 | (AE003429) CG2934 gene product [Drosophila |
| | | | | | | | melanogaster] |
| 2340 | LIB3602-109-Q1-K1-E11 | 7442647 | BLASTX | 212 | 6.00E−22 | 37 | hypothetical protein F14M4.17 - Arabidopsis |
| | | | | | | | thaliana >gi\|3522943\|gb\|AAC34225.1\| (AC004411) |
| | | | | | | | putative ABC transporter [Arabidopsis thaliana] |
| 2341 | LIB3602-067-Q1-K1-A9 | 115385 | BLASTX | 243 | 3.00E−20 | 48 | CHLOROPHYLL A-B BINDING PROTEIN 4 |
| | | | | | | | PRECURSOR (LHCI TYPE III CAB-4) (LHCP) |
| | | | | | | | >gi\|166646\|gb\|AAA32760.1\| (M63931) light- |
| | | | | | | | harvesting chlorophyll a/b binding protein |
| | | | | | | | [Arabidopsis thaliana] |
| | | | | | | | >gi\|6522530\|emb\|CAB61973.1\| (AL132955) |
| | | | | | | | CHLOROPHYLL A-B BINDING PROTEIN 4 PR |
| 2342 | LIB3602-011-Q6-K1-G9 | 7488383 | BLASTX | 202 | 2.00E−15 | 40 | transcription factor IID 30K chain homolog |
| | | | | | | | F28M20.90 - Arabidopsis thaliana |
| | | | | | | | >gi\|2072626\|emb\|CAA73389.1\| (Y12904) |
| | | | | | | | hypothetical protein [Arabidopsis thaliana] |
| | | | | | | | >gi\|3281856\|emb\|CAA19751.1\| (AL031004) |
| | | | | | | | Transcription factor II homolog [Arabidopsis |
| | | | | | | | thaliana] >gi\| |
| 2343 | LIB3602-023-Q6-K1-F8 | 7489278 | BLASTX | 254 | 1.00E−21 | 36 | thioredoxin-like protein CDSP32, chloroplast - |
| | | | | | | | potato >gi\|2582822\|emb\|CAA71103.1\| (Y09987) |
| | | | | | | | CDSP32 protein (Chloroplast Drought-induced |
| | | | | | | | Stress Protein of 32 kDa) [Solanum tuberosum] |
| 2344 | LIB3602-107-Q1-K1-G9 | 2129726 | BLASTX | 467 | 2.00E−46 | 51 | DNA-directed RNA polymerase (EC 2.7.7.6) II |
| | | | | | | | 35.5K chain A - Arabidopsis thaliana |
| | | | | | | | >gi\|514318\|gb\|AAB03741.1\| (L34770) RNA |
| | | | | | | | polymerase II third largest subunit [Arabidopsis |
| | | | | | | | thaliana] |
| | | | | | | | >gi\|4544370\|gb\|AAD22281.1\|AC006920_5 |
| | | | | | | | (AC006920) DNA-directed RNA polymerase |
| 2345 | LIB3602-017-Q6-K1-A1 | 7488371 | BLASTX | 329 | 1.00E−30 | 66 | thioredoxin reductase homolog T32G6.20 - |
| | | | | | | | Arabidopsis thaliana >gi\|2618704\|gb\|AAB84351.1\| |
| | | | | | | | (AC002510) putative thioredoxin reductase |
| | | | | | | | [Arabidopsis thaliana] |
| 2346 | LIB3602-080-Q6-K6-A6 | 4887543 | BLASTX | 195 | 1.00E−14 | 75 | (AJ012278) ATP-dependent Clp protease subunit |
| | | | | | | | ClpP [Arabidopsis thaliana] |
| | | | | | | | >gi\|5360579\|dbj\|BAA82065.1\| (AB022326) nClpP1 |
| | | | | | | | [Arabidopsis thaliana] |
| 2347 | LIB3602-045-Q6-K1-F6 | 7469824 | BLASTX | 195 | 1.00E−14 | 32 | hypothetical protein - Synechocystis sp. (strain PCC |
| | | | | | | | 6803) >gi\|1653953\|dbj\|BAA18863.1\| (D90917) |
| | | | | | | | hypothetical protein [Synechocystis sp.] |
| 2348 | LIB3602-030-Q6-K1-E1 | 7485867 | BLASTX | 211 | 1.00E−16 | 37 | hypothetical protein F20D22.10 - Arabidopsis |
| | | | | | | | thaliana >gi\|3142297\|gb\|AAC16748.1\| (AC002411) |
| | | | | | | | Contains similarity to serine/threonine protein |
| | | | | | | | phosphatase gb\|X83099 from S. cerevisiae. |
| | | | | | | | [Arabidopsis thaliana] |
| 2349 | LIB3602-085-Q6-K1-C8 | 7302133 | BLASTX | 251 | 3.00E−21 | 35 | (AE003781) CG1416 gene product [Drosophila |
| | | | | | | | melanogaster] |
| 2350 | LIB3602-055-Q6-K1-D12 | 2351580 | BLASTX | 317 | 5.00E−29 | 60 | (U82433) thymidine diphospho-glucose 4-6- |
| | | | | | | | dehydratase homolog [Prunus armeniaca] |
| 2351 | LIB3602-077-Q6-K6-C11 | 7462403 | BLASTX | 464 | 4.00E−46 | 56 | heat shock protein HslV - Thermotoga maritima |
| | | | | | | | (strain MSB8) |
| | | | | | | | >gi\|4981034\|gb\|AAD35606.1\|AE001728_7 |
| | | | | | | | (AE001728) heat shock protein HslV [Thermotoga |
| | | | | | | | maritima] |
| 2352 | LIB3602-040-Q6-K1-A9 | 2494744 | BLASTX | 400 | 8.00E−39 | 85 | GLUTAMINE SYNTHETASE CYTOSOLIC |
| | | | | | | | ISOZYME (GLUTAMATE--AMMONIA LIGASE) |
| | | | | | | | (GS1) >gi\|7437922\|pir\|\|T08088 glutamate-- |
| | | | | | | | ammonia ligase (EC 6.3.1.2), cytosolic - |
| | | | | | | | Chlamydomonas reinhardtii |
| | | | | | | | >gi\|1353876\|gb\|AAB01817.1\| (U46207) glutamine |
| | | | | | | | synthetase [Chlamydomonas reinhard |
| 2353 | LIB3602-012-Q6-K1-B3 | 122805 | BLASTX | 180 | 7.00E−13 | 32 | 6-HYDROXY-D-NICOTINE OXIDASE (6- |
| | | | | | | | HDNO) >gi\|580720\|emb\|CAA29416.1\| (X05999) 6- |
| | | | | | | | hydroxy-D-nicotine oxidase [Arthrobacter oxidans] |
| 2354 | LIB3602-049-Q6-K1-B9 | 99772 | BLASTX | 209 | 2.00E−16 | 68 | ubiquitin/ribosomal protein S27a.2 - Arabidopsis |
| | | | | | | | thaliana >gi\|166936\|gb\|AAA32907.1\| (J05540) |
| | | | | | | | ubiquitin extension protein (UBQ6) [Arabidopsis |
| | | | | | | | thaliana] >gi\|3522953\|gb\|AAC34235.1\| |
| | | | | | | | (AC004411) ubiquitin extension protein (UBQ6) |
| | | | | | | | [Arabidopsis thaliana] |
| 2355 | LIB3602-103-Q1-K1-F9 | 6679160 | BLASTX | 216 | 3.00E−17 | 39 | nucleoredoxin >gi\|1854550\|emb\|CAA63408.1\| |
| | | | | | | | (X92750) red-1 [Mus musculus] |
| 2356 | LIB3602-023-Q6-K1-D9 | 7594543 | BLASTX | 300 | 6.00E−27 | 41 | (AL163972) putative protein [Arabidopsis thaliana] |
| 2357 | LIB3602-087-Q6-K1-F10 | 7489244 | BLASTX | 275 | 1.00E−32 | 57 | hypothetical protein R1 - potato |
| | | | | | | | >gi\|3287270\|emb\|CAA70725.1\| (Y09533) involved |
| | | | | | | | in starch metabalism [Solanum tuberosum] |
| 2358 | LIB3602-118-Q1-K1-H4 | 7019967 | BLASTX | 156 | 3.00E−10 | 53 | (AK000099) unnamed protein product [Homo |
| | | | | | | | sapiens] |
| 2359 | LIB3602-115-Q1-K1-B7 | 1729938 | BLASTX | 185 | 2.00E−13 | 45 | THIOREDOXIN-LIKE PROTEIN SLR0233 |
| | | | | | | | >gi\|7430831\|pir\|\|S76386 thioredoxin M-1 - |
| | | | | | | | Synechocystis sp. (strain PCC 6803) |
| | | | | | | | >gi\|1001610\|dbj\|BAA10238.1\| (D64000) |
| | | | | | | | thioredoxin M [Synechocystis sp.] |
| 2360 | LIB3602-093-Q6-K6-A4 | 7225945 | BLASTX | 175 | 2.00E−12 | 41 | (AE002426) queuine tRNA-ribosyltransferase |
| | | | | | | | [Neisseria meningitidis] |
| 2361 | LIB3602-023-Q6-K1-F12 | 595780 | BLASTX | 159 | 2.00E−10 | 43 | (U13871) lacZ alpha peptide [unidentified cloning |
| | | | | | | | vector] |
| 2362 | LIB3602-008-Q6-K1-E11 | 7023065 | BLASTX | 606 | 7.00E−63 | 66 | (AK001667) unnamed protein product [Homo |
| | | | | | | | sapiens] |
| 2363 | LIB3602-111-Q1-K1-B3 | 6651403 | BLASTX | 159 | 1.00E−16 | 29 | (AF180734) dynamin-like protein 5 [Arabidopsis |
| | | | | | | | thaliana] |
| 2364 | LIB3602-018-Q6-K1-G8 | 6562305 | BLASTX | 259 | 3.00E−22 | 42 | (AL133421) putative protein [Arabidopsis thaliana] |
| 2365 | LIB3602-014-Q6-K1-F7 | 6562306 | BLASTX | 219 | 1.00E−17 | 45 | (AL133421) diacylglycerol kinase (ATDGK1) |
| | | | | | | | [Arabidopsis thaliana] |
| 2366 | LIB3602-016-Q6-K1-C10 | 1729930 | BLASTX | 204 | 8.00E−16 | 43 | THREONINE DEHYDRATASE BIOSYNTHETIC |
| | | | | | | | (THREONINE DEAMINASE) |
| | | | | | | | >gi\|1113117\|gb\|AAA83215.1\| (U40630) L- |
| | | | | | | | threonine deaminase [Burkholderia cepacia] |
| 2367 | LIB3602-115-Q1-K1-H4 | 7432656 | BLASTX | 158 | 3.00E−10 | 40 | NADH dehydrogenase (ubiquinone) (EC 1.6.5.3) - |
| | | | | | | | fission yeast (Schizosaccharomyces pombe) |
| | | | | | | | >gi\|2414635\|emb\|CAB16382.1\| (Z99260) putative |
| | | | | | | | nadh-dehydrogenase [Schizosaccharomyces pombe] |
| 2368 | LIB3602-012-Q6-K1-F5 | 7489245 | BLASTX | 241 | 2.00E−20 | 50 | kinesin heavy chain-like protein (clone PKCBP) - |
| | | | | | | | potato >gi\|1369852\|gb\|AAB37756.1\| (L46702) |
| | | | | | | | kinesin heavy chain-like protein [Solanum |
| | | | | | | | tuberosum] |
| 2369 | LIB3602-034-Q6-K1-B9 | 7428204 | BLASTX | 353 | 4.00E−33 | 51 | carboxypeptidase D (EC 3.4.16.6) precursor - barley |
| | | | | | | | >gi\|1731990\|emb\|CAA70815.1\| (Y09602) serine |
| | | | | | | | carboxypeptidase II, CP-MII [Hordeum vulgare] |
| 2370 | LIB3602-007-Q1-K1-A11 | 2498140 | BLASTX | 181 | 6.00E−13 | 41 | APAG PROTEIN (CORD PROTEIN) |
| | | | | | | | >gi\|1003021\|gb\|AAA79339.1\| (U24176) CorD |
| | | | | | | | [Salmonella typhimurium] |
| 2371 | LIB3602-076-Q6-K6-F10 | 7433551 | BLASTX | 749 | 1.00E−79 | 71 | glycine hydroxymethyltransferase (EC 2.1.2.1) |
| | | | | | | | F20D10.50 - Arabidopsis thaliana |
| | | | | | | | >gi\|4467099\|emb\|CAB37533.1\| (AL035538) |
| | | | | | | | glycine hydroxymethyltransferase like protein |
| | | | | | | | [Arabidopsis thaliana] |
| | | | | | | | >gi\|6899945\|emb\|CAB71289.1\| (AJ271726) serine |
| | | | | | | | hydroxymethyl transfer |
| 2372 | LIB3602-001-P1-K6-F1 | 4006886 | BLASTX | 284 | 4.00E−25 | 53 | (Z99708) putative protein [Arabidopsis thaliana] |
| | | | | | | | >gi\|7270635\|emb\|CAB80352.1\| (AL161590) |
| | | | | | | | putative protein [Arabidopsis thaliana] |
| 2373 | LIB3602-041-Q6-K1-E7 | 6562319 | BLASTX | 173 | 5.00E−12 | 35 | (AL133421) putative protein [Arabidopsis thaliana] |
| 2374 | LIB3602-064-Q1-K6-B10 | 1350977 | BLASTX | 144 | 2.00E−12 | 71 | PROBABLE 40S RIBOSOMAL PROTEIN S30 |
| 2375 | LIB3602-002-P1-K6-F7 | 2924772 | BLASTX | 203 | 1.00E−15 | 48 | (AC002334) unknown protein [Arabidopsis |
| | | | | | | | thaliana] |
| 2376 | LIB3602-011-Q6-K1-C5 | 2506275 | BLASTX | 199 | 3.00E−19 | 52 | CHAPERONIN CPN60-1, MITOCHONDRIAL |
| | | | | | | | PRECURSOR (HSP60-1) |
| 2377 | LIB3602-084-Q6-K1-G5 | 6692692 | BLASTX | 310 | 4.00E−28 | 54 | (AC007592) F12K11.10 [Arabidopsis thaliana] |
| 2378 | LIB3602-047-Q6-K1-E3 | 5523979 | BLASTX | 252 | 2.00E−21 | 54 | (AF104025) polyprotein [Bovine viral diarrhea |
| | | | | | | | virus-2] |
| 2379 | LIB3602-077-Q6-K6-D2 | 7485688 | BLASTX | 283 | 3.00E−25 | 60 | hypothetical protein F18A5.110 - Arabidopsis |
| | | | | | | | thaliana >gi\|4455301\|emb\|CAB36836.1\| |
| | | | | | | | (AL035528) putative protein [Arabidopsis thaliana] |
| | | | | | | | >gi\|7268076\|emb\|CAB78414.1\| (AL161537) |
| | | | | | | | putative protein [Arabidopsis thaliana] |
| 2380 | LIB3602-100-Q1-K1-E6 | 7298579 | BLASTX | 431 | 3.00E−51 | 56 | (AE003662) CG9987 gene product [Drosophila |
| | | | | | | | melanogaster] |
| 2381 | LIB3602-095-Q6-K6-E4 | 3327204 | BLASTX | 465 | 3.00E−46 | 52 | (AB014595) KIAA0695 protein [Homo sapiens] |
| 2382 | LIB3602-095-Q6-K6-D5 | 3327206 | BLASTX | 199 | 4.00E−15 | 37 | (AB014596) KIAA0696 protein [Homo sapiens] |
| 2383 | LIB3602-036-Q6-K1-E10 | 7300338 | BLASTX | 181 | 5.00E−13 | 41 | (AE003721) CG7217 gene product [Drosophila |
| | | | | | | | melanogaster] |
| 2384 | LIB3602-066-Q1-K6-E11 | 7488307 | BLASTX | 162 | 8.00E−11 | 47 | ribosomal protein L14 homolog T24A18.40 - |
| | | | | | | | Arabidopsis thaliana |
| | | | | | | | >gi\|4490705\|emb\|CAB38839.1\| (AL035680) |
| | | | | | | | ribosomal protein L14-like protein [Arabidopsis |
| | | | | | | | thaliana] >gi\|7269562\|emb\|CAB79564.1\| |
| | | | | | | | (AL161566) ribosomal protein L14-like protein |
| | | | | | | | [Arabidopsis thaliana |
| 2385 | LIB3602-040-Q6-K1-E12 | 5757449 | BLASTX | 157 | 2.00E−10 | 73 | (AB021315) L41 ribosomal protein [Coprinus |
| | | | | | | | cinereus] |
| 2386 | LIB3602-013-Q6-K1-D9 | 7437876 | BLASTX | 285 | 1.00E−25 | 64 | probable acid - CoA ligase (EC 6.2.1.—) fadD35 - |
| | | | | | | | Mycobacterium tuberculosis (strain H37RV) |
| | | | | | | | >gi\|2113938\|emb\|CAB08923.1\| (Z95556) fadD35 |
| | | | | | | | [Mycobacterium tuberculosis] |
| 2387 | LIB3602-042-Q6-K1-D5 | 2499954 | BLASTX | 331 | 1.00E−30 | 54 | URACIL PHOSPHORIBOSYLTRANSFERASE |
| | | | | | | | (UMP PYROPHOSPHORYLASE) (UPRTASE) |
| | | | | | | | >gi\|7427938\|pir\|\|S74616 uracil |
| | | | | | | | phosphoribosyltransferase (EC 2.4.2.9) upp - |
| | | | | | | | Synechocystis sp. (strain PCC 6803) |
| | | | | | | | >gi\|1651841\|dbj\|BAA16768.1\| (D90900) uracil |
| | | | | | | | phosphoribosyltransferase [Synec |
| 2388 | LIB3602-012-Q6-K1-F7 | 81235 | BLASTX | 731 | 2.00E−77 | 76 | chlorophyll a/b-binding protein - Chlamydomonas |
| | | | | | | | reinhardtii |
| 2389 | LIB3602-055-Q6-K1-C1 | 5902062 | BLASTX | 364 | 1.00E−34 | 62 | polymerase (RNA) III (DNA directed) (155 kD) |
| | | | | | | | >gi\|3122765\|sp\|O14802\|RPC1_HUMAN DNA- |
| | | | | | | | DIRECTED RNA POLYMERASE III LARGEST |
| | | | | | | | SUBUNIT (RPC155) >gi\|2460208\|gb\|AAB86536.1\| |
| | | | | | | | (AF021351) RNA polymerase III largest subunit |
| | | | | | | | [Homo sapiens] |
| 2390 | LIB3602-091-Q6-K6-H1 | 5454054 | BLASTX | 162 | 9.00E−11 | 35 | splicing factor, arginine/serine-rich 7 (35 kD) |
| | | | | | | | >gi\|3929380\|sp\|Q16629\|SFR7_HUMAN SPLICING |
| | | | | | | | FACTOR, ARGININE/SERINE-RICH 7 |
| | | | | | | | (SPLICING FACTOR 9G8) |
| | | | | | | | >gi\|2136194\|pir\|\|A57198 splicing factor, |
| | | | | | | | arginine/serine-rich 7 - human |
| | | | | | | | >gi\|506402\|gb\|AAA35495.1\| (L22253) 9G8 sp |
| 2391 | LIB3602-065-Q1-K6-G12 | 3982576 | BLASTX | 803 | 7.00E−86 | 76 | (AF023140) imidazoleglycerol phosphate |
| | | | | | | | dehydratase [Thlaspi goesingense] |
| 2392 | LIB3602-083-Q6-K6-G7 | 6226702 | BLASTX | 555 | 8.00E−57 | 62 | 40S RIBOSOMAL PROTEIN S8 |
| | | | | | | | >gi\|7440237\|pir$$ ribosomal protein S8 - |
| | | | | | | | maize >gi\|14980$$AB06330.1\| (U64436) |
| | | | | | | | ribosomal protein S8 [Zea mays] |
| 2393 | LIB3602-003-Q1-K1-E4 | 2464901 | BLASTX | 232 | 4.00E−19 | 41 | (Z99708) putative protein [Arabidopsis thaliana] |
| | | | | | | | >gi\|7270628\|emb\|CAB80345.1\| (AL161590) |
| | | | | | | | putative protein [Arabidopsis thaliana] |
| 2394 | LIB3602-009-Q6-K1-C9 | 3372671 | BLASTX | 162 | 5.00E−11 | 54 | (AF061286) gamma-adaptin 1 [Arabidopsis |
| | | | | | | | thaliana] |
| | | | | | | | >gi\|4704741\|gb\|AAD28247.1\|AF124524_1 |
| | | | | | | | $$124524$$tin 1 [Arabidopsis |
| | | | | | | | thaliana] |
| 2395 | LIB3602-041-Q6-K1-C1 | 7512095 | BLASTX | 175 | 3.00E−12 | 33 | scavenger receptor cysteine-rich protein precursor - |
| | | | | | | | sea urchin (Strongylocentrotus purpuratus) |
| | | | | | | | >gi\|4165053\|gb\|AAD08654.1\| (AF064259) |
| | | | | | | | scavenger receptor cysteine-rich protein type 12 |
| | | | | | | | precursor [Strongylocentrotus purpuratus] |
| 2396 | LIB3602-074-Q1-K1-B5 | 6225655 | BLASTX | 328 | 3.00E−30 | 51 | MALATE SYNTHASE |
| | | | | | | | >gi\|2811132\|gb\|AAB97829.1\| (AF013216) Mls |
| | | | | | | | [Myxococcus xanthus] |
| 2397 | LIB3602-078-Q6-K6-B4 | 7297250 | BLASTX | 292 | 6.00E−26 | 61 | (AE003617) CG5261 gene product [alt 2] |
| | | | | | | | [Drosophila melanogaster] |
| 2398 | LIB3602-101-Q1-K1-D3 | 7436983 | BLASTX | 404 | 3.00E−50 | 79 | probable imidazoleglycerol-phosphate dehydratase |
| | | | | | | | (EC 4.2.1.19) - wheat (fragment) |
| | | | | | | | >gi\|551331\|gb\|AAA93197.1\| (U02690) |
| | | | | | | | imidazoleglycerolphosphate dehydratase [Triticum |
| | | | | | | | aestivum] |
| 2399 | LIB3602-001-P1-K6-G7 | 6056371 | BLASTX | 653 | 3.00E−68 | 77 | (AC009894) nucleolar protein [Arabidopsis |
| | | | | | | | thaliana] |
| 2400 | LIB3602-051-Q6-K1-B1 | 2116650 | BLASTX | 757 | 9.00E−84 | 94 | (AB004044) alpha-1-antitrypsin [Cercopithecus |
| | | | | | | | aethiops] |
| 2401 | LIB3602-092-Q6-K6-C2 | 6900314 | BLASTX | 238 | 1.00E−19 | 81 | (AJ250663) putative proteinase inhibitor [Hordeum |
| | | | | | | | vulgare] |
| 2402 | LIB3602-062-Q6-K1-F10 | 113324 | BLASTX | 271 | 1.00E−23 | 57 | ACYLPHOSPHATASE, MUSCLE TYPE |
| | | | | | | | ISOZYME (ACYLPHOSPHATE |
| | | | | | | | PHOSPHOHYDROLASE) (ISOZYME TU1) |
| | | | | | | | >gi\|67797\|pir\|\|QPTK acylphosphatase (EC 3.6.1.7) |
| | | | | | | | Tu1, skeletal muscle - turkey |
| | | | | | | | >gi\|223821\|prf\|\|1001154A acylphosphatase |
| | | | | | | | [Meleagris gallopavo] |
| 2403 | LIB3602-115-Q1-K1-D12 | 3293555 | BLASTX | 712 | 3.00E−75 | 70 | (AF072931) chlorophyll a/b binding protein |
| | | | | | | | [Medicago sativa] |
| 2404 | LIB3602-062-Q6-K1-D4 | 7225376 | BLASTX | 177 | 7.00E−13 | 64 | (AE002373) 50S ribosomal protein L18 [Neisseria |
| | | | | | | | meningitidis] >gi\|7378885\|emb\|CAB83428.1\| |
| | | | | | | | (AL162752) 50S ribosomal protein L18 [Neisseria |
| | | | | | | | meningitidis] |
| 2405 | LIB3602-016-Q6-K1-E8 | 1707981 | BLASTX | 259 | 3.00E−22 | 59 | GLUTAREDOXIN >gi\|1076561\|pir\|\|S54825 |
| | | | | | | | glutaredoxin - castor bean |
| 2406 | LIB3602-014-Q6-K1-B10 | 6754812 | BLASTX | 179 | 9.00E−13 | 51 | NADH dehydrogenase (ubiquinone) 1 alpha |
| | | | | | | | subcomplex 2 (8 kDa) |
| | | | | | | | >gi\|4836511\|gb\|AAD30475.1\|AF124786_1 |
| | | | | | | | (AF124786) NADH-ubiquinone oxidoreductase B8 |
| | | | | | | | subunit [Mus musculus] |
| 2407 | LIB3602-095-Q6-K6-B5 | 1408302 | BLASTX | 348 | 1.00E−43 | 43 | (U61987) phosphoprotein phosphatase A |
| | | | | | | | [Dictyostelium discoideum] |
| 2408 | LIB3602-061-Q6-K1-H3 | 7510484 | BLASTX | 151 | 1.00E−09 | 44 | hypothetical protein Y75B8A.26 - Caenorhabditis |
| | | | | | | | elegans >gi\|3980042\|emb\|CAA22110.1\| |
| | | | | | | | (AL033514) similar to ABC transporters; ABC |
| | | | | | | | transporter transmembrane region. [Caenorhabditis |
| | | | | | | | elegans] |
| 2409 | LIB3602-025-Q6-K1-G6 | 5381319 | BLASTX | 658 | 5.00E−69 | 79 | (AF091621) ubiquitin-conjugating enzyme E2 |
| | | | | | | | [Catharanthus roseus] |
| 2410 | LIB3602-069-Q1-K1-A6 | 2565436 | BLASTX | 562 | 1.00E−57 | 58 | (AF028842) DegP protease precursor [Arabidopsis |
| | | | | | | | thaliana] |
| 2411 | LIB3602-094-Q6-K6-C8 | 4587571 | BLASTX | 277 | 2.00E−24 | 50 | (AC006550) Belongs to the PF\|01027 |
| | | | | | | | Uncharacterized protein family UPF0005 with 7 |
| | | | | | | | transmembrane domains. [Arabidopsis thaliana] |
| 2412 | LIB3602-045-Q6-K1-C7 | 7484375 | BLASTX | 310 | 3.00E−28 | 49 | envelope protein LIP-36G2, low CO2 inducible - |
| | | | | | | | Chlamydomonas reinhardtii |
| | | | | | | | >gi\|2459575\|gb\|AAB71744.1\| (U75346) envelope |
| | | | | | | | protein [Chlamydomonas reinhardtii] |
| 2413 | LIB3602-061-Q6-K1-G7 | 4775576 | BLASTX | 591 | 4.00E−61 | 67 | (AJ238785) MUS1 protein [Zea mays] |
| 2414 | LIB3602-076-Q6-K6-B6 | 2499966 | BLASTX | 162 | 1.00E−10 | 50 | PHOTOSYSTEM I REACTION CENTRE |
| | | | | | | | SUBUNIT IV A PRECURSOR (PSI-E A) |
| | | | | | | | >gi\|7443146\|pir\|\|T16962 photosystem I chain PSI-E - |
| | | | | | | | wood tobacco >gi\|632722\|gb\|AAB31704.1\| |
| | | | | | | | (S72356) photosystem I subunit PSI-E [Nicotiana |
| | | | | | | | sylvestris, leaves, Peptide Chloroplast, 141 aa] |
| 2415 | LIB3602-002-P1-K6-B7 | 3025293 | BLASTX | 259 | 3.00E−22 | 83 | RAS-RELATED PROTEIN YPTC6 |
| | | | | | | | >gi\|1363476\|pir\|\|JC4108 GTP-binding protein |
| | | | | | | | yptC6 - Chlamydomonas reinhardtii |
| | | | | | | | >gi\|806728\|gb\|AAA82729.1\| (U13169) YptC6 |
| | | | | | | | [Chlamydomonas reinhardtii] |
| 2416 | LIB3602-115-Q1-K1-G6 | 6652886 | BLASTX | 255 | 7.00E−22 | 80 | (AF123394) 26S proteasome AAA-ATPase subunit |
| | | | | | | | RPT5a [Arabidopsis thaliana] |
| | | | | | | | >gi\|7596759\|gb\|AAF64530.1\| (AC009606) 26S |
| | | | | | | | proteasome AAA-ATPase subunit RPT5a |
| | | | | | | | [Arabidopsis thaliana] |
| 2417 | LIB3602-052-Q6-K1-F6 | 7489990 | BLASTX | 441 | 1.00E−43 | 63 | 40s ribosomal protein s4-2 - fission yeast |
| | | | | | | | (Schizosaccharomyces pombe) |
| | | | | | | | >gi\|6018736\|emb\|CAB57920.1\| (AL121794) 40s |
| | | | | | | | ribosomal protein s4-2 [Schizosaccharomyces |
| | | | | | | | pombe] |
| 2418 | LIB3602-076-Q6-K6-F7 | 3121830 | BLASTX | 725 | 9.00E−77 | 67 | CHROMATIN ASSEMBLY FACTOR 1 P55 |
| | | | | | | | SUBUNIT (CAF-1 P55 SUBUNIT) (DCAF-1) |
| | | | | | | | (NUCLEOSOME REMODELING FACTOR 55 KD |
| | | | | | | | SUBUNIT) (NURF-55) |
| | | | | | | | >gi\|1407828\|gb\|AAB37257.1\| (U62388) chromatin |
| | | | | | | | assembly factor 1 p55 subunit [Drosophila |
| | | | | | | | melanogaster] >gi\|7299974\|gb\|AAF55146.1\| (A |
| 2419 | LIB3602-052-Q6-K1-C5 | 3123614 | BLASTX | 678 | 2.00E−71 | 79 | (Y17225) cyclin-dependent protein kinase p34cdc2 |
| | | | | | | | [Lycopersicon esculentum] |
| 2420 | LIB3602-106-Q1-K1-F1 | 3121836 | BLASTX | 295 | 2.00E−26 | 74 | PHOSPHATIDATE CYTIDYLYLTRANSFERASE |
| | | | | | | | (CDP-DIGLYCERIDE SYNTHETASE) (CDP- |
| | | | | | | | DIGLYCERIDE PYROPHOSPHORYLASE) |
| | | | | | | | (CDP-DIACYLGLYCEROL SYNTHASE) (CDS) |
| | | | | | | | (CTP: PHOSPHATIDATE |
| | | | | | | | CYTIDYLYLTRANSFERASE) (CDP-DAG |
| | | | | | | | SYNTHASE) >gi\|2181182\|emb\|CAA63969.1\| |
| | | | | | | | (X94306) CDP-diacylglycerol syn |
| 2421 | LIB3602-027-Q6-K1-H1 | 2213584 | BLASTX | 192 | 2.00E−14 | 50 | (AC000348) T7N9.4 [Arabidopsis thaliana] |
| 2422 | LIB3602-112-Q1-K1-F9 | 6729509 | BLASTX | 345 | 3.00E−32 | 68 | (AL132966) diaminopimelate epimerase-like |
| | | | | | | | protein [Arabidopsis thaliana] |
| 2423 | LIB3602-067-Q1-K1-D8 | 710550 | BLASTX | 214 | 8.00E−17 | 39 | (L40632) ankyrin 3 [Mus musculus] |
| 2424 | LIB3602-010-Q6-K1-H2 | 710551 | BLASTX | 246 | 1.00E−20 | 37 | (L40632) ankyrin 3 [Mus musculus] |
| 2425 | LIB3602-047-Q6-K1-H2 | 6652880 | BLASTX | 704 | 2.00E−74 | 93 | (AF123391) 26S proteasome AAA-ATPase subunit |
| | | | | | | | RPT2a [Arabidopsis thaliana] |
| 2426 | LIB3602-027-Q6-K1-G10 | 4406787 | BLASTX | 160 | 1.00E−10 | 59 | (AC006532) unknown protein [Arabidopsis |
| | | | | | | | thaliana] |
| 2427 | LIB3602-059-Q6-K1-H1 | 5926716 | BLASTX | 412 | 4.00E−40 | 57 | (AB005903) AtPH1 [Arabidopsis thaliana] |
| 2428 | LIB3602-077-Q6-K6-C6 | 7484346 | BLASTX | 728 | 4.00E−77 | 68 | phytoene synthase (EC 2.5.1.—) - green alga |
| | | | | | | | (Dunaliella bardawil) >gi\|1916990\|gb\|AAB51287.1\| |
| | | | | | | | (U91900) phytoene synthase [Dunaliella bardawil] |
| 2429 | LIB3602-027-Q6-K1-A3 | 6539563 | BLASTX | 325 | 6.00E−30 | 47 | (AP000836) Similar to ISP42-like protein |
| | | | | | | | (AF051231) [Oryza sativa] |
| 2430 | LIB3602-003-Q1-K1-F3 | 1906804 | BLASTX | 254 | 9.00E−22 | 46 | (D87750) farnesyl cysteine carboxyl |
| | | | | | | | methyltransferase [Xenopus laevis] |
| 2431 | LIB3602-057-Q6-K1-F5 | 6755196 | BLASTX | 151 | 3.00E−19 | 66 | proteasome (prosome, macropain) subunit, alpha |
| | | | | | | | type 4 >gi\|5757663\|gb\|AAD50538.1\|AF060093_1 |
| | | | | | | | (AF060093) proteasome subunit C9 [Mus |
| | | | | | | | musculus] |
| 2432 | LIB3602-003-Q1-K1-D2 | 4874296 | BLASTX | 146 | 3.00E−11 | 51 | (AC007443) putative chlorophyll a/b binding |
| | | | | | | | protein [Arabidopsis thaliana] |
| 2433 | LIB3602-019-Q6-K1-D6 | 3894197 | BLASTX | 249 | 5.00E−21 | 68 | (AC005662) hypothetical protein [Arabidopsis |
| | | | | | | | thaliana] |
| 2434 | LIB3602-030-Q6-K1-D4 | 7448559 | BLASTX | 312 | 2.00E−28 | 63 | glutamate N-acetyltransferase (EC 2.3.1.35) - |
| | | | | | | | Synechocystis sp. (strain PCC 6803) |
| | | | | | | | >gi\|1653293\|dbj\|BAA18208.1\| (D90912) ornithine |
| | | | | | | | acetyltransferase [Synechocystis sp.] |
| 2435 | LIB3602-013-Q6-K1-C11 | 2688839 | BLASTX | 144 | 8.00E−09 | 60 | (AF003347) ATP phosphoribosyltransferase |
| | | | | | | | [Thlaspi goesingense] |
| 2436 | LIB3602-062-Q6-K1-H7 | 7630021 | BLASTX | 289 | 1.00E−25 | 35 | (AL132960) pasticcino 1 [Arabidopsis thaliana] |
| 2437 | LIB3602-070-Q1-K1-D7 | 1173223 | BLASTX | 152 | 6.00E−13 | 64 | 40S RIBOSOMAL PROTEIN S11 |
| | | | | | | | >gi\|454848\|gb\|AAC14469.1\| (L28831) ribosomal |
| | | | | | | | protein S11 [Glycine max] |
| 2438 | LIB3602-055-Q6-K1-D6 | 4506589 | BLASTX | 267 | 3.00E−23 | 39 | RNA 3′-terminal phosphate cyclase |
| | | | | | | | >gi\|2500648\|sp\|O00442\|RTC1_HUMAN RNA 3′- |
| | | | | | | | TERMINAL PHOSPHATE CYCLASE 1 (RNA-3′- |
| | | | | | | | PHOSPHATE CYCLASE 1) (RNA CYCLASE 1) |
| | | | | | | | >gi\|2125812\|emb\|CAA72364.1\| (Y11651) |
| | | | | | | | phosphate cyclase [Homo sapiens] |
| 2439 | LIB3602-076-Q6-K6-H9 | 7404436 | BLASTX | 156 | 4.00E−10 | 56 | 60S RIBOSOMAL PROTEIN L6 |
| | | | | | | | >gi\|7490046\|pir\|\|T41499 60s ribosomal protein 16 - |
| | | | | | | | fission yeast (Schizosaccharomyces pombe) |
| | | | | | | | >gi\|3821337\|emb\|CAA21874.1\| (AL033127) 60s |
| | | | | | | | ribosomal protein 16 [Schizosaccharomyces pombe] |
| 2440 | LIB3602-087-Q6-K1-C3 | 7485401 | BLASTX | 145 | 5.00E−09 | 62 | hypothetical protein F12C20.6 - Arabidopsis |
| | | | | | | | thaliana >gi\|3426051\|gb\|AAC32250.1\| (AC005168) |
| | | | | | | | putative Na+ dependent ileal bile acid transporter |
| | | | | | | | [Arabidopsis thaliana] |
| 2441 | LIB3602-016-Q6-K1-A4 | 3157934 | BLASTX | 523 | 3.00E−53 | 66 | (AC002131) Similar to hypothetical protein |
| | | | | | | | F09E5.8 gb\|U37429 from C. elegans. ESTs |
| | | | | | | | gb\|T42019 and gb\|N97000 come from this gene. |
| | | | | | | | [Arabidopsis thaliana] |
| 2442 | LIB3602-019-Q6-K1-E9 | 267002 | BLASTX | 261 | 2.00E−22 | 50 | SULFITE REDUCTASE (FERREDOXIN) |
| | | | | | | | >gi\|66219\|pir\|\|RDYCS7 sulfite reductase |
| | | | | | | | (ferredoxin) (EC 1.8.7.1) - Synechococcus sp |
| | | | | | | | >gi\|38930\|emb\|CAA77809.1\| (Z11755) ferredoxin- |
| | | | | | | | sulfite reductase [Synechococcus PCC6301] |
| 2443 | LIB3602-026-Q6-K1-A4 | 7480951 | BLASTX | 591 | 4.00E−61 | 64 | probable long-chain-fatty-acid-CoA ligase - |
| | | | | | | | Streptomyces coelicolor |
| | | | | | | | >gi\|4455741\|emb\|CAB36604.1\| (AL035478) |
| | | | | | | | putative long-chain-fatty-acid-CoA ligase |
| | | | | | | | [Streptomyces coelicolor A3(2)] |
| 2444 | LIB3602-059-Q6-K1-D4 | 6729515 | BLASTX | 244 | 2.00E−20 | 44 | (AL132966) protein kinase-like protein |
| | | | | | | | [Arabidopsis thaliana] |
| 2445 | LIB3602-067-Q1-K1-C4 | 4836870 | BLASTX | 408 | 1.00E−39 | 56 | (AC007260) 50S Ribosomal protein L13 |
| | | | | | | | [Arabidopsis thaliana] |
| | | | | | | | >gi\|7572938\|emb\|CAA60775.1\| (X87333) |
| | | | | | | | ribosomal protein L13 [Arabidopsis thaliana] |
| 2446 | LIB3602-043-Q6-K1-F8 | 7447850 | BLASTX | 351 | 4.00E−33 | 80 | hypothetical protein F3L17.50 - Arabidopsis |
| | | | | | | | thaliana >gi\|5262759\|emb\|CAB45907.1\| |
| | | | | | | | (AL080283) putative protein [Arabidopsis thaliana] |
| | | | | | | | >gi\|7270051\|emb\|CAB79866.1\| (AL161579) |
| | | | | | | | putative protein [Arabidopsis thaliana] |
| 2447 | LIB3602-042-Q6-K1-H3 | 7413562 | BLASTX | 230 | 9.00E−19 | 53 | (AL162973) 60S ribosomal protein-like |
| | | | | | | | [Arabidopsis thaliana] |
| 2448 | LIB3602-048-Q6-K1QA-G9 | 7630237 | BLASTX | 352 | 4.00E−33 | 44 | (AP001859) ESTs |
| | | | | | | | AU082761(S5084), D42006(S5084) correspond to a |
| | | | | | | | region of the predicted gene. ˜Similar to |
| | | | | | | | Caenorhabditis elegans chromosome 3, cosmid |
| | | | | | | | C17G10; alcohol dehydrogenase/ribitol |
| | | | | | | | dehydrogenase. (U28739) [Oryza sativa] |
| 2449 | LIB3602-061-Q6-K1-E5 | 3122785 | BLASTX | 591 | 4.00E−61 | 86 | 40S RIBOSOMAL PROTEIN S14 |
| | | | | | | | >gi\|7440341\|pir\|\|T07974 ribosomal protein S14 - |
| | | | | | | | yellow lupine >gi\|2565340\|gb\|AAB81972.1\| |
| | | | | | | | (AF026079) ribosomal protein S14 [Lupinus luteus] |
| 2450 | LIB3602-047-Q6-K1-A6 | 6524184 | BLASTX | 318 | 4.00E−29 | 49 | (AC011914) putative mutT protein [Arabidopsis |
| | | | | | | | thaliana] |
| 2451 | LIB3602-011-Q6-K1-G8 | 5262856 | BLASTX | 281 | 9.00E−25 | 60 | (AJ238095) Lsm3 protein [Homo sapiens] |
| | | | | | | | >gi\|5919147\|gb\|AAD56227.1\|AF182289_1 |
| | | | | | | | (AF182289) U6 snRNA-associated Sm-like protein |
| | | | | | | | LSm3 [Homo sapiens] |
| 2452 | LIB3602-034-Q6-K1-B7 | 5757653 | BLASTX | 149 | 2.00E−09 | 43 | (AF060088) proteasome subunit C2 [Mus |
| | | | | | | | musculus] |
| 2453 | LIB3602-066-Q1-K6-F12 | 5007086 | BLASTX | 449 | 2.00E−44 | 59 | (AF155334) NADP-specific isocitrate |
| | | | | | | | dehydrogenase [Oryza sativa] |
| 2454 | LIB3602-026-Q6-K1-G2 | 730536 | BLASTX | 179 | 2.00E−13 | 84 | 60S RIBOSOMAL PROTEIN L23 |
| | | | | | | | >gi\|7440849\|pir\|\|T03693 ribosomal protein L17 - |
| | | | | | | | common tobacco >gi\|310933\|gb\|AAA34113.1\| |
| | | | | | | | (L18915) 60S ribosomal protein subunit L17 |
| | | | | | | | [Nicotiana tabacum] |
| 2455 | LIB3602-059-Q6-K1-B4 | 7492749 | BLASTX | 281 | 9.00E−25 | 45 | probable nadh-cytochrome b5 reductase - fission |
| | | | | | | | yeast (Schizosaccharomyces pombe) |
| | | | | | | | >gi\|3560228\|emb\|CAA20696.1\| (AL031530) |
| | | | | | | | putative nadh-cytochrome b5 reductase |
| | | | | | | | [Schizosaccharomyces pombe] |
| 2456 | LIB3602-040-Q6-K1-F10 | 416583 | BLASTX | 241 | 4.00E−20 | 75 | ACETYL-COENZYME A SYNTHETASE |
| | | | | | | | (ACETATE--COA LIGASE) (ACYL- |
| | | | | | | | ACTIVATING ENZYME) |
| | | | | | | | >gi\|7437823\|pir\|\|D65215 acetate - CoA ligase (EC |
| | | | | | | | 6.2.1.1) - Escherichia coli |
| | | | | | | | >gi\|396404\|gb\|AAC43163.1\| (U00006) acetyl--CoA |
| | | | | | | | sythetase [Escherichia coli] |
| | | | | | | | >gi\|1790505\|gb\|AAC77039.1\| ( |
| 2457 | LIB3602-092-Q6-K6-A2 | 551288 | BLASTX | 710 | 5.00E−75 | 88 | (Z33611) phosphoglycerate mutase [Zea mays] |
| 2458 | LIB3602-066-Q1-K6-B11 | 3599489 | BLASTX | 162 | 9.00E−11 | 40 | (AF085148) 3-oxoacyl-[acyl-carrier-protein] |
| | | | | | | | synthase [Capsicum chinense] |
| 2459 | LIB3602-085-Q6-K1-H3 | 7021222 | BLASTX | 219 | 5.00E−25 | 56 | (AF059510) 3-methylcrotonyl-CoA carboxylase |
| | | | | | | | non-biotinylated subunit [Arabidopsis thaliana] |
| | | | | | | | >gi\|7021224\|gb\|AAF35259.1\|AF059511_1 |
| | | | | | | | (AF059511) 3-methylcrotonyl-CoA carboxylase |
| | | | | | | | nonbiotinylated subunit [Arabidopsis thaliana] |
| 2460 | LIB3602-029-Q6-K1-E2 | 1703091 | BLASTX | 169 | 9.00E−12 | 71 | ACYL CARRIER PROTEIN, MITOCHONDRIAL |
| | | | | | | | PRECURSOR (ACP) (NADH-UBIQUINONE |
| | | | | | | | OXIDOREDUCTASE 9.6 KD SUBUNIT) |
| | | | | | | | (MTACP-1) >gi\|7442278\|pir\|\|T01589 probable acyl |
| | | | | | | | carrier protein F16B22.11 - Arabidopsis thaliana |
| | | | | | | | >gi\|903689\|gb\|AAB96840.1\| (L23574) acyl carrier |
| | | | | | | | protein pr |
| 2461 | LIB3602-027-Q6-K1-E4 | 7469427 | BLASTX | 177 | 1.00E−12 | 33 | hypothetical protein - Synechocystis sp. (strain PCC |
| | | | | | | | 6803) >gi\|1001449\|dbj\|BAA10074.1\| (D63999) |
| | | | | | | | hypothetical protein [Synechocystis sp.] |
| 2462 | LIB3602-011-Q6-K1-A7 | 7431396 | BLASTX | 242 | 3.00E−20 | 61 | xanthine dehydrogenase homolog T11I11.140 - |
| | | | | | | | Arabidopsis thaliana |
| | | | | | | | >gi\|5123707\|emb\|CAB45451.1\| (AL079347) |
| | | | | | | | xanthine dehydrogenase [Arabidopsis thaliana] |
| | | | | | | | >gi\|7270441\|emb\|CAB80207.1 \| (AL161586) |
| | | | | | | | xanthine dehydrogenase [Arabidopsis thaliana] |
| 2463 | LIB3602-002-P1-K6-G12 | 7406448 | BLASTX | 147 | 6.00E−09 | 40 | (AL162875) putative protein [Arabidopsis thaliana] |
| 2464 | LIB3602-042-Q6-K1-A7 | 2853277 | BLASTX | 152 | 7.00E−10 | 60 | (U82756) WD splicing factor Hprp4p [Homo |
| | | | | | | | sapiens] |
| 2465 | LIB3602-070-Q1-K1-G3 | 7431559 | BLASTX | 502 | 1.00E−50 | 73 | hypothetical protein - Synechocystis sp. (strain PCC |
| | | | | | | | 6803) >gi\|1653680\|dbj\|BAA18592.1\| (D90915) |
| | | | | | | | pyruvate dehydrogenase E1 component, alpha |
| | | | | | | | subunit [Synechocystis sp.] |
| 2466 | LIB3602-025-Q6-K1-E2 | 132076 | BLASTX | 523 | 4.00E−53 | 67 | RIBULOSE BISPHOSPHATE CARBOXYLASE |
| | | | | | | | SMALL CHAIN 1 PRECURSOR (RUBISCO |
| | | | | | | | SMALL SUBUNIT 1) >gi\|68109\|pir\|\|RKKMS1 |
| | | | | | | | ribulose-bisphosphate carboxylase (EC 4.1.1.39) |
| | | | | | | | small chain 1 precursor - Chlamydomonas |
| | | | | | | | reinhardtii |
| 2467 | LIB3602-046-Q6-K1-B9 | 7486523 | BLASTX | 231 | 3.00E−19 | 45 | hypothetical protein F6H11.40 - Arabidopsis |
| | | | | | | | thaliana >gi\|2827702\|emb\|CAA16675.1\| |
| | | | | | | | (AL021684) predicted protein [Arabidopsis |
| | | | | | | | thaliana] |
| 2468 | LIB3602-109-Q1-K1-F4 | 7298362 | BLASTX | 152 | 1.00E−09 | 51 | (AE003653) ApepP gene product [Drosophila |
| | | | | | | | melanogaster] |
| 2469 | LIB3602-030-Q6-K1-H3 | 6066383 | BLASTX | 414 | 2.00E−40 | 51 | (AJ011926) Mg-protoporphyrin IX [Hordeum |
| | | | | | | | vulgare] |
| 2470 | LIB3602-112-Q1-K1-B4 | 1905979 | BLASTX | 219 | 2.00E−17 | 46 | (U81052) oligosaccharyltransferase subunit |
| | | | | | | | homolog [Mus musculus] |
| 2471 | LIB3602-041-Q6-K1-H6 | 167433 | BLASTN | 34 | 3.00E−09 | 89 | C. reinhardtii ribulose 1,5-bisphosphate |
| | | | | | | | carboxylase/oxygenase activase mRNA, complete |
| | | | | | | | cds |
| 2472 | LIB3602-025-Q6-K1-A2 | 7485629 | BLASTX | 365 | 1.00E−34 | 57 | hypothetical protein F17K2.15 - Arabidopsis |
| | | | | | | | thaliana >gi\|2979552\|gb\|AAC06161.1\| (AC003680) |
| | | | | | | | unknown protein [Arabidopsis thaliana] |
| 2473 | LIB3602-015-Q6-K1-G7 | 129699 | BLASTX | 582 | 5.00E−60 | 71 | PROLIFERATING CELL NUCLEAR ANTIGEN |
| | | | | | | | (PCNA) (CYCLIN) >gi\|99946\|pir\|\|S14414 |
| | | | | | | | proliferating cell nuclear antigen - soybean |
| | | | | | | | (fragment) >gi\|18726\|emb\|CAA39239.1\| (X55706) |
| | | | | | | | proliferating cell nuclear antigen [Glycine max] |
| 2474 | LIB3602-046-Q6-K1-B4 | 3122952 | BLASTX | 144 | 8.00E−09 | 32 | TIPD PROTEIN >gi\|7489899\|pir\|\|T08602 protein |
| | | | | | | | TipD - slime mold (Dictyostelium discoideum) |
| | | | | | | | >gi\|2407788\|gb\|AAB70659.1\| (AF019236) TipD |
| | | | | | | | [Dictyostelium discoideum] |
| 2475 | LIB3602-025-Q6-K1-B7 | 2773404 | BLASTX | 324 | 8.00E−30 | 46 | (AF041255) pyridoxal kinase [Sus scrofa] |
| 2476 | LIB3602-118-Q1-K1-D4 | 7380038 | BLASTX | 167 | 7.00E−21 | 60 | (AL162755) putative GTP-binding protein |
| | | | | | | | [Neisseria meningitidis] |
| 2477 | LIB3602-010-Q6-K1-A3 | 7487074 | BLASTX | 556 | 5.00E−57 | 61 | hypothetical protein T16H5.240 - Arabidopsis |
| | | | | | | | thaliana (fragment) >gi\|3250697\|emb\|CAA19705.1\| |
| | | | | | | | (AL024486) putative protein [Arabidopsis thaliana] |
| | | | | | | | >gi\|7268784\|emb\|CAB78990.1\| (AL161551) |
| | | | | | | | putative protein [Arabidopsis thaliana] |
| 2478 | LIB3602-062-Q6-K1-F2 | 7021047 | BLASTX | 174 | 3.00E−12 | 41 | (AK000759) unnamed protein product [Homo |
| | | | | | | | sapiens] |
| 2479 | LIB3602-108-Q1-K1-B4 | 7503535 | BLASTX | 181 | 4.00E−13 | 34 | hypothetical protein F46B3.9 - Caenorhabditis |
| | | | | | | | elegans >gi\|3877232\|emb\|CAB04398.1\| (Z81540) |
| | | | | | | | predicted using Genefinder [Caenorhabditis |
| | | | | | | | elegans] |
| 2480 | LIB3602-070-Q1-K1-C8 | 7630059 | BLASTX | 227 | 2.00E−18 | 33 | (AL353013) putative protein [Arabidopsis thaliana] |
| 2481 | LIB3602-090-Q6-K6-A3 | 6226735 | BLASTX | 199 | 4.00E−15 | 37 | PUTATIVE ACETYLTRANSFERASE |
| | | | | | | | C18B11.09C IN CHROMOSOME I |
| | | | | | | | >gi\|7492354\|pir\|\|T37905 probable acetyltransferase - |
| | | | | | | | fission yeast (Schizosaccharomyces pombe) |
| | | | | | | | >gi\|4150918\|emb\|CAA90593.1\| (Z50728) putative |
| | | | | | | | acetyltransferase [Schizosaccharomyces pombe] |
| 2482 | LIB3602-052-Q6-K1-B8 | 7486331 | BLASTX | 175 | 2.00E−12 | 52 | hypothetical protein F28M20.60 - Arabidopsis |
| | | | | | | | thaliana >gi\|3281853\|emb\|CAA19748.1\| |
| | | | | | | | (AL031004) putative protein [Arabidopsis thaliana] |
| | | | | | | | >gi\|7270078\|emb\|CAB79893.1\| (AL161579) |
| | | | | | | | putative protein [Arabidopsis thaliana] |
| 2483 | LIB3602-086-Q6-K1-B7 | 6970282 | BLASTX | 323 | 1.00E−29 | 64 | (AC021666) unknown protein [Arabidopsis |
| | | | | | | | thaliana] |
| 2484 | LIB3602-016-Q6-K1-A6 | 1707992 | BLASTX | 280 | 1.00E−24 | 51 | SERINE HYDROXYMETHYLTRANSFERASE |
| | | | | | | | (SERINE METHYLASE) (GLYCINE |
| | | | | | | | HYDROXYMETHYLTRANSFERASE) (SHMT) |
| | | | | | | | >gi\|485099\|gb\|AAB53830.1\| (U00048) Contains |
| | | | | | | | similarity to Pfam domain: PF00464 (SHMT), |
| | | | | | | | Score = 954.1, E-value = 9.9e−287, N = 1 |
| | | | | | | | [Caenorhabditis elegans] |
| 2485 | LIB3602-114-Q1-K1-F4 | 226788 | BLASTX | 166 | 3.00E−11 | 35 | erythrocyte ankyrin [Homo sapiens] |
| 2486 | LIB3602-100-Q1-K1-C4 | 7019397 | BLASTX | 150 | 2.00E−09 | 34 | GDP-mannose pyrophosphorylase B |
| | | | | | | | >gi\|5052351\|gb\|AAD38516.1\|AF135421_1 |
| | | | | | | | (AF135421) GDP-mannose pyrophosphorylase B |
| | | | | | | | [Homo sapiens] |
| 2487 | LIB3602-008-Q6-K1-G8 | 6056388 | BLASTX | 790 | 2.00E−84 | 94 | (AC009324) 26S proteasome ATPase subunit |
| | | | | | | | [Arabidopsis thaliana] |
| 2488 | LIB3602-085-Q6-K1-H6 | 4263697 | BLASTX | 185 | 9.00E−14 | 46 | (AC006223) 60S ribosomal protein L27 |
| | | | | | | | [Arabidopsis thaliana] |
| 2489 | LIB3602-029-Q6-K1-G2 | 7484385 | BLASTX | 267 | 4.00E−23 | 68 | L-ascorbate peroxidase (EC 1.11.1.11) precursor - |
| | | | | | | | Chlamydomonas reinhardtii |
| | | | | | | | >gi\|2887338\|emb\|CAA11265.1\| (AJ223325) |
| | | | | | | | ascorbate peroxidase [Chlamydomonas reinhardtii] |
| 2490 | LIB3602-115-Q1-K1-E2 | 7466728 | BLASTX | 173 | 4.00E−12 | 39 | probable iron-sulfur-binding protein b0947 - |
| | | | | | | | Escherichia coli >gi\|1787179\|gb\|AAC74033.1\| |
| | | | | | | | (AE000196) orf, hypothetical protein [Escherichia |
| | | | | | | | coli] |
| 2491 | LIB3602-100-Q1-K1-A4 | 7487059 | BLASTX | 180 | 5.00E−13 | 38 | hypothetical protein T16B24.22 - Arabidopsis |
| | | | | | | | thaliana >gi\|3402689\|gb\|AAC28992.1\| (AC004697) |
| | | | | | | | unknown protein [Arabidopsis thaliana] |
| 2492 | LIB3602-059-Q6-K1-C8 | 6978917 | BLASTX | 327 | 2.00E−30 | 60 | (AC021665) putative histidinol-phosphate |
| | | | | | | | aminotransferase [Arabidopsis thaliana] |
| 2493 | LIB3602-040-Q6-K1-H7 | 6807962 | BLASTX | 461 | 4.00E−46 | 72 | (AL137410) hypothetical protein [Homo sapiens] |
| 2494 | LIB3602-115-Q1-K1-B9 | 7340654 | BLASTX | 550 | 3.00E−63 | 68 | (AL162506) fructose-bisphosphate aldolase-like |
| | | | | | | | protein [Arabidopsis thaliana] |
| 2495 | LIB3602-029-Q6-K1-G11 | 7630068 | BLASTX | 206 | 6.00E−16 | 71 | (AL353032) v-ATPase subunit D (vATPD) |
| | | | | | | | [Arabidopsis thaliana] |
| 2496 | LIB3602-066-Q1-K6-C5 | 122090 | BLASTX | 426 | 1.00E−41 | 98 | HISTONE H3 >gi\|81287\|pir\|\|S00940 histone H3 - |
| | | | | | | | Volvox carteri >gi\|2119017\|pir\|\|S59581 histone H3 |
| | | | | | | | (clones CH-II and CH-III) - Chlamydomonas |
| | | | | | | | reinhardtii >gi\|21985\|emb\|CAA30035.1\| (X06963) |
| | | | | | | | put. histone H3 [Volvox carteri] |
| | | | | | | | >gi\|21988\|emb\|CAA30037.1\| (X06964) pu |
| 2497 | LIB3602-037-Q6-K1-G8 | 7206844 | BLASTX | 187 | 6.00E−14 | 50 | (AC006834) contains similarity to NADH- |
| | | | | | | | ubiquinone oxidoreductase 18 KD subunits |
| | | | | | | | [Caenorhabditis elegans] |
| 2498 | LIB3602-052-Q6-K1-A7 | 6165265 | BLASTX | 341 | 7.00E−32 | 62 | (AJ250733) translation elongation factor 1-alpha |
| | | | | | | | [Dreissena polymorpha] |
| 2499 | LIB3602-118-Q1-K1-E1 | 7576173 | BLASTX | 158 | 6.00E−14 | 33 | (AL163912) putative protein [Arabidopsis thaliana] |
| 2500 | LIB3602-108-Q1-K1-D10 | 4689334 | BLASTX | 271 | 8.00E−29 | 63 | (AF132555) BcDNA.GM07659 [Drosophila |
| | | | | | | | melanogaster] |
| 2501 | LIB3602-030-Q6-K1-D2 | 7630065 | BLASTX | 580 | 7.00E−60 | 77 | (AL353032) ribosomal protein L11-like |
| | | | | | | | [Arabidopsis thaliana] |
| 2502 | LIB3602-053-Q6-K1-B1 | 6056399 | BLASTX | 188 | 8.00E−14 | 61 | (AC009324) AP2 domain containing protein |
| | | | | | | | RAP2.12 [Arabidopsis thaliana] |
| 2503 | LIB3602-111-Q1-K1-C12 | 7485448 | BLASTX | 273 | 8.00E−24 | 44 | hypothetical protein F13M23.170 - Arabidopsis |
| | | | | | | | thaliana >gi\|4455246\|emb\|CAB36745.1\| |
| | | | | | | | (AL035523) putative protein [Arabidopsis thaliana] |
| | | | | | | | >gi\|7269353\|emb\|CAB79412.1\| (AL161562) |
| | | | | | | | putative protein [Arabidopsis thaliana] |
| 2504 | LIB3602-038-Q6-K1-H11 | 167253 | BLASTX | 849 | 2.00E−91 | 94 | (M74441) ribulose 1,5-bisphosphate carboxylase |
| | | | | | | | large subunit [Chlorella-like algae] |
| 2505 | LIB3602-042-Q6-K1-C2 | 4689328 | BLASTX | 408 | 1.00E−39 | 59 | (AF132552) BcDNA.GM01838 [Drosophila |
| | | | | | | | melanogaster] >gi\|7301261\|gb\|AAF56391.1\| |
| | | | | | | | (AE003750) OstStt3 gene product [Drosophila |
| | | | | | | | melanogaster] |
| 2506 | LIB3602-039-Q6-K1-G11 | 4585973 | BLASTX | 241 | 5.00E−20 | 66 | (AC005287) translation initiation factor |
| | | | | | | | [Arabidopsis thaliana] |
| 2507 | LIB3602-018-Q6-K1-H1 | 4587598 | BLASTX | 246 | 1.00E−22 | 47 | (AC006951) 3-oxoacyl carrier protein synthase |
| | | | | | | | [Arabidopsis thaliana] |
| 2508 | LIB3602-087-Q6-K1-A10 | 65068 | BLASTX | 274 | 5.00E−24 | 50 | (X05025) ribosomal protein L14 [Xenopus laevis] |
| 2509 | LIB3602-051-Q6-K1-G7 | 1220453 | BLASTX | 383 | 9.00E−37 | 47 | (M79328) alpha-amylase [Solanum tuberosum] |
| 2510 | LIB3602-041-Q6-K1-B12 | 7473394 | BLASTX | 361 | 1.00E−34 | 65 | probable acyl-CoA dehydrogenase - Deinococcus |
| | | | | | | | radiodurans (strain R1) |
| | | | | | | | >gi\|6460476\|gb\|AAF12182.1\|AE001862_8 |
| | | | | | | | (AE001862) acyl-CoA dehydrogenase, putative |
| | | | | | | | [Deinococcus radiodurans] |
| 2511 | LIB3602-072-Q1-K1-B12 | 7437759 | BLASTX | 510 | 2.00E−51 | 51 | arginine - tRNA ligase (EC 6.1.1.19) - Arabidopsis |
| | | | | | | | thaliana >gi\|2632105\|emb\|CAB11468.1\| (Z98760) |
| | | | | | | | arginyl-tRNA synthetase [Arabidopsis thaliana] |
| | | | | | | | >gi\|4539426\|emb\|CAB38959.1\| (AL049171) |
| | | | | | | | arginyl-tRNA synthetase [Arabidopsis thaliana] |
| | | | | | | | >gi\|7269482\|emb\|CAB79485.1 |
| 2512 | LIB3602-104-Q1-K1-H8 | 3421104 | BLASTX | 450 | 1.00E−44 | 71 | (AF043531) 20S proteasome beta subunit PBB2 |
| | | | | | | | [Arabidopsis thaliana] |
| 2513 | LIB3602-004-Q1-K1-E10 | 3212610 | BLASTX | 386 | 5.00E−37 | 57 | Chain A, Sulfite Oxidase From Chicken Liver |
| | | | | | | | >gi\|3212611\|pdb\|1SOX\|B Chain B, Sulfite Oxidase |
| | | | | | | | From Chicken Liver |
| 2514 | LIB3602-003-Q1-K1-G7 | 3041702 | BLASTX | 205 | 8.00E−16 | 30 | TRIACYLGLYCEROL LIPASE, GASTRIC |
| | | | | | | | PRECURSOR (GASTRIC LIPASE) (GL) |
| | | | | | | | >gi\|2204113\|emb\|CAA74198.1\| (Y13899) |
| | | | | | | | triacylglycerol lipase [Canis familiaris] |
| 2515 | LIB3602-064-Q1-K6-A9 | 2500381 | BLASTX | 270 | 1.00E−26 | 65 | 60S RIBOSOMAL PROTEIN L44 P (L41) (L41 P- |
| | | | | | | | TYPE) |
| 2516 | LIB3602-079-Q6-K6-B4 | 3025188 | BLASTX | 285 | 4.00E−25 | 52 | HYPOTHETICAL 11.9 KD PROTEIN SLR1846 |
| | | | | | | | >gi\|7448385\|pir\|\|S75164 hypothetical protein |
| | | | | | | | slr1846 - Synechocystis sp. (strain PCC 6803) |
| | | | | | | | >gi\|1652154\|dbj\|BAA17078.1\| (D90903) |
| | | | | | | | hypothetical protein [Synechocystis sp.] |
| 2517 | LIB3602-059-Q6-K1-G8 | 4587605 | BLASTX | 224 | 5.00E−18 | 41 | (AC006951) putative mutT domain protein |
| | | | | | | | [Arabidopsis thaliana] |
| 2518 | LIB3602-089-Q6-K6-H5 | 21983 | BLASTN | 64 | 3.00E−27 | 97 | Volvox carteri H3-I and H4-I genes for histones H3 |
| | | | | | | | and H4 |
| 2519 | LIB3602-030-Q6-K1-C7 | 4263711 | BLASTX | 515 | 3.00E−52 | 63 | (AC006223) putative CCR4-associated factor |
| | | | | | | | [Arabidopsis thaliana] |
| 2520 | LIB3602-084-Q6-K1-G12 | 6056404 | BLASTX | 409 | 1.00E−39 | 50 | (AC009525) Similar to cullin proteins [Arabidopsis |
| | | | | | | | thaliana] |
| 2521 | LIB3602-039-Q6-K1-B9 | 4263714 | BLASTX | 540 | 4.00E−55 | 59 | (AC006223) unknown protein [Arabidopsis |
| | | | | | | | thaliana] |
| 2522 | LIB3602-105-Q1-K1-E2 | 2583129 | BLASTX | 621 | 1.00E−64 | 64 | (AC002387) putative methionine aminopeptidase |
| | | | | | | | [Arabidopsis thaliana] |
| 2523 | LIB3602-113-Q1-K1-B2 | 1931647 | BLASTX | 573 | 6.00E−59 | 56 | (U95973) endomembrane protein EMP70 precusor |
| | | | | | | | isolog [Arabidopsis thaliana] |
| 2524 | LIB3602-105-Q1-K1-H5 | 6649217 | BLASTX | 199 | 4.00E−15 | 50 | (AF165422) suppressor of K+ transport growth |
| | | | | | | | defect-like protein [Mesembryanthemum |
| | | | | | | | crystallinum] |
| 2525 | LIB3602-101-Q1-K1-D5 | 7573355 | BLASTX | 156 | 3.00E−10 | 61 | (AL163812) diaminopimelate decarboxylase-like |
| | | | | | | | protein [Arabidopsis thaliana] |
| 2526 | LIB3602-094-Q6-K6-F11 | 6119520 | BLASTX | 517 | 2.00E−52 | 50 | (AC011560) unknown protein [Arabidopsis |
| | | | | | | | thaliana] |
| 2527 | LIB3602-017-Q6-K1-D10 | 1583601 | BLASTX | 200 | 2.00E−15 | 76 | zeta carotene desaturase [Capsicum annuum] |
| 2528 | LIB3602-051-Q6-K1-C11 | 3122638 | BLASTX | 345 | 4.00E−49 | 66 | PP1/PP2A PHOSPHATASES PLEIOTROPIC |
| | | | | | | | REGULATOR PRL1 >gi\|1076381\|pir\|\|S49820 |
| | | | | | | | PRL1 protein - Arabidopsis thaliana |
| | | | | | | | >gi\|577733\|emb\|CAA58031.1\| (X82824) PRL1 |
| | | | | | | | [Arabidopsis thaliana] |
| | | | | | | | >gi\|577735\|emb\|CAA58032.1\| (X82825) PRL1 |
| | | | | | | | [Arabidopsis thaliana] >gi\|2244947\|emb\|CA |
| 2529 | LIB3602-046-Q6-K1-G12 | 2501449 | BLASTX | 228 | 1.00E−18 | 47 | UBIQUITIN-LIKE PROTEIN SMT3 |
| | | | | | | | >gi\|7446816\|pir\|\|T04102 smt3 protein - rice |
| | | | | | | | >gi\|1668773\|emb\|CAA67922.1\| (X99608) |
| | | | | | | | ubiquitin-like protein [Oryza sativa] |
| 2530 | LIB3602-106-Q1-K1-C4 | 1323548 | BLASTN | 70 | 1.00E−30 | 90 | Chlamydomonas reinhardtii carbonic anhydrase |
| | | | | | | | precursor (beta-CA1) mRNA, nuclear gene |
| | | | | | | | encoding mitochondrial protein, complete cds |
| 2531 | LIB3602-103-Q1-K1-H4 | 6321942 | BLASTX | 300 | 5.00E−27 | 61 | ribosomal protein (weak similarity); Imp3p |
| | | | | | | | >gi\|418596\|sp\|P32899\|IMP3_YEAST U3 SMALL |
| | | | | | | | NUCLEOLAR RIBONUCLEOPROTEIN |
| | | | | | | | PROTEIN IMP3 >gi\|485540\|pir\|\|S33911 probable |
| | | | | | | | ribosomal protein YHR148w - yeast |
| | | | | | | | (Saccharomyces cerevisiae) |
| | | | | | | | >gi\|4014\|emb\|CAA49237.1\| (X69480) uOR |
| 2532 | LIB3602-057-Q6-K1-E2 | 6119523 | BLASTX | 275 | 6.00E−24 | 32 | (AC011560) putative amino acid transporter |
| | | | | | | | [Arabidopsis thaliana] |
| 2533 | LIB3602-052-Q6-K1-C11 | 7431230 | BLASTX | 184 | 1.00E−13 | 50 | malate dehydrogenase (oxaloacetate- |
| | | | | | | | decarboxylating) (NADP+) (EC 1.1.1.40) - tomato |
| | | | | | | | (fragment) >gi\|804817\|gb\|AAA66051.1\| (L36647) |
| | | | | | | | malic enzyme [Lycopersicon esculentum] |
| 2534 | LIB3602-109-Q1-K1-G7 | 3264759 | BLASTX | 444 | 5.00E−44 | 70 | (AF071889) 40S ribosomal protein S8 [Prunus |
| | | | | | | | armeniaca] |
| 2535 | LIB3602-068-Q1-K1-G3 | 7475184 | BLASTX | 260 | 3.00E−22 | 35 | hypothetical protein ymaE - Bacillus subtilis |
| | | | | | | | >gi\|2634109\|emb\|CAB13609.1\| (Z99113) ymaE |
| | | | | | | | [Bacillus subtilis] |
| 2536 | LIB3602-044-Q6-K1-H6 | 4506489 | BLASTX | 411 | 5.00E−40 | 57 | replication factor C (activator 1) 3 (38 kD) |
| | | | | | | | >gi\|3915601\|sp\|P40938\|AC13_HUMAN |
| | | | | | | | ACTIVATOR 1 38 KD SUBUNIT |
| | | | | | | | (REPLICATION FACTOR C 38 KD SUBUNIT) |
| | | | | | | | (A1 38 KD SUBUNIT) (RF-C 38 KD SUBUNIT) |
| | | | | | | | (RFC38) >gi\|7444557\|pir\|\|T09573 replication factor |
| | | | | | | | C 38K chain - human >gi\| |
| 2537 | LIB3602-040-Q6-K1-D11 | 7486039 | BLASTX | 321 | 2.00E−29 | 40 | hypothetical protein F23F1.14 - Arabidopsis |
| | | | | | | | thaliana >gi\|3420057\|gb\|AAC31858.1\| (AC004680) |
| | | | | | | | putative ABC transporter [Arabidopsis thaliana] |
| 2538 | LIB3602-004-Q1-K1-F10 | 7446679 | BLASTX | 152 | 3.00E−15 | 49 | peptide methionine sulfoxide reductase - |
| | | | | | | | Methanobacterium thermoautotrophicum (strain |
| | | | | | | | Delta H) >gi\|2621609\|gb\|AAB85041.1\| (AE000836) |
| | | | | | | | peptide methionine sulfoxide reductase |
| | | | | | | | [Methanobacterium thermoautotrophicum] |
| 2539 | LIB3602-014-Q6-K1-D10 | 6716785 | BLASTX | 632 | 7.00E−66 | 90 | (AF220539) 40s ribosomal protein S23 [Euphorbia |
| | | | | | | | esula] |
| 2540 | LIB3602-057-Q6-K1-D4 | 4826972 | BLASTX | 315 | 1.00E−28 | 44 | RNA binding motif protein 8 |
| | | | | | | | >gi\|4455129\|gb\|AAD21089.1\| (AF127761) |
| | | | | | | | ribonucleoprotein RBM8 [Homo sapiens] |
| | | | | | | | >gi\|6841450\|gb\|AAF29078.1\|AF161463_1 |
| | | | | | | | (AF161463) HSPC114 [Homo sapiens] |
| 2541 | LIB3602-016-Q6-K1-E6 | 2274859 | BLASTX | 318 | 4.00E−29 | 74 | (AJ000016) Cks1 protein [Arabidopsis thaliana] |
| | | | | | | | >gi\|4510420\|gb\|AAD21506.1\| (AC006929) putative |
| | | | | | | | cyclin-dependent kinase regulatory subunit |
| | | | | | | | [Arabidopsis thaliana] |
| 2542 | LIB3602-041-Q6-K1-G1 | 2160174 | BLASTX | 572 | 8.00E−59 | 62 | (AC000132) Identical to A. thaliana PUR2 |
| | | | | | | | (gb\|X74766). ESTs gb\|ATTS3927, gb\|N96446 come |
| | | | | | | | from this gene. [Arabidopsis thaliana] |
| 2543 | LIB3602-106-Q1-K1-H11 | 5281051 | BLASTX | 296 | 2.00E−26 | 49 | (AL080318) stress-induced protein sti1-like protein |
| | | | | | | | [Arabidopsis thaliana] |
| | | | | | | | >gi\|7267942\|emb\|CAB78283.1\| (AL161534) stress- |
| | | | | | | | induced protein sti1-like protein [Arabidopsis |
| | | | | | | | thaliana] |
| 2544 | LIB3602-062-Q6-K1-G6 | 7288201 | BLASTX | 320 | 3.00E−29 | 68 | (AF108451) phosphoglycerate kinase precursor |
| | | | | | | | [Phaeodactylum tricornutum] |
| 2545 | LIB3602-066-Q1-K6-E1 | 7305087 | BLASTX | 394 | 6.00E−38 | 54 | glutamine fructose-6-phosphate transaminase 2 |
| | | | | | | | >gi\|6226821\|sp\|Q9Z2Z9\|GFA2_MOUSE |
| | | | | | | | GLUCOSAMINE-FRUCTOSE-6-PHOSPHATE |
| | | | | | | | AMINOTRANSFERASE [ISOMERIZING] 2 |
| | | | | | | | (HEXOSEPHOSPHATE AMINOTRANSFERASE |
| | | | | | | | 2) (D-FRUCTOSE-6-PHOSPHATE |
| | | | | | | | AMIDOTRANSFERASE 2) (GFAT 2) (GFAT2) |
| | | | | | | | >gi\|4239879 |
| 2546 | LIB3602-053-Q6-K1-A9 | 7490623 | BLASTX | 185 | 1.00E−13 | 45 | heat shock protein 70 family - fission yeast |
| | | | | | | | (Schizosaccharomyces pombe) |
| | | | | | | | >gi\|2992154\|dbj\|BAA25322.1\| (AB012387) heat |
| | | | | | | | shock protein [Schizosaccharomyces pombe] |
| | | | | | | | >gi\|3581889\|emb\|CAA20787.1\| (AL031540) heat |
| | | | | | | | shock protein 70 family [Schizosaccharomyces |
| | | | | | | | pombe] |
| 2547 | LIB3602-016-Q6-K1-F3 | 4826940 | BLASTX | 156 | 8.00E−15 | 40 | Pro-X carboxypeptidase precursor |
| | | | | | | | >gi\|1172047\|sp\|P42785\|PCP_HUMAN |
| | | | | | | | LYSOSOMAL PRO-X CARBOXYPEPTIDASE |
| | | | | | | | PRECURSOR (PROLYLCARBOXYPEPTIDASE) |
| | | | | | | | (PRCP) (PROLINE CARBOXYPEPTIDASE) |
| | | | | | | | (ANGIOTENSINASE C) (LYSOSOMAL |
| | | | | | | | CARBOXYPEPTIDASE C) |
| | | | | | | | >gi\|476943\|pir\|\|A47352 lysosomal Pro-X |
| 2548 | LIB3602-048-Q6-K1QA- | 3219858 | BLASTX | 245 | 1.00E−20 | 45 | DNA-DIRECTED RNA POLYMERASE II 13.6 KD |
| | D10 | | | | | | POLYPEPTIDE >gi\|2129724\|pir\|\|S71204 |
| | | | | | | | DNA-directed RNA polymerase (EC 2.7.7.6) II |
| | | | | | | | 13.6K chain - Arabidopsis thaliana |
| | | | | | | | >gi\|881501\|gb\|AAB02849.1\| (U28048) RNA |
| | | | | | | | polymerase II 13.6 kDa subunit [Arabidopsis |
| | | | | | | | thaliana] >gi\|4678 |
| 2549 | LIB3602-022-Q6-K1-D12 | 7435799 | BLASTX | 317 | 4.00E−29 | 46 | cysteine proteinase (EC 3.4.22.—) - barley |
| | | | | | | | >gi\|2224810\|emb\|CAB09698.1\| (Z97022) cysteine |
| | | | | | | | proteinase [Hordeum vulgare] |
| 2550 | LIB3602-023-Q6-K1-H2 | 2213612 | BLASTX | 201 | 2.00E−15 | 40 | (AC000103) F21J9.6 [Arabidopsis thaliana] |
| 2551 | LIB3602-010-Q6-K1-E7 | 4512684 | BLASTX | 265 | 7.00E−23 | 61 | (AC006931) unknown protein [Arabidopsis |
| | | | | | | | thaliana] |
| 2552 | LIB3602-042-Q6-K1-A5 | 6692118 | BLASTX | 570 | 1.00E−58 | 89 | (AC007764) F22C12.2 [Arabidopsis thaliana] |
| 2553 | LIB3602-054-Q6-K1-A10 | 5921971 | BLASTX | 469 | 7.00E−47 | 55 | (AF106575) contains similarity to Pfam domain |
| | | | | | | | PF00171 (aldedh), Score = 286.5, E-value = 1.1e−82, |
| | | | | | | | N = 1 [Caenorhabditis elegans] |
| 2554 | LIB3602-062-Q6-K1-H1 | 7267544 | BLASTX | 649 | 7.00E−68 | 64 | (AL161513) isoamylase-like protein [Arabidopsis |
| | | | | | | | thaliana] |
| 2555 | LIB3602-064-Q1-K6-G11 | 4679028 | BLASTX | 145 | 1.00E−08 | 28 | (AF077207) HSPC021 [Homo sapiens] |
| | | | | | | | >gi\|5106781\|gb\|AAD39841.1\| (AF083243) |
| | | | | | | | HSPC025 [Homo sapiens] |
| 2556 | LIB3602-022-Q6-K1-B1 | 6735297 | BLASTX | 290 | 6.00E−26 | 42 | (AL137080) putative protein [Arabidopsis thaliana] |
| 2557 | LIB3602-116-Q1-K1-A11 | 2500352 | BLASTX | 300 | 6.00E−46 | 58 | 60S RIBOSOMAL PROTEIN L10 |
| | | | | | | | >gi\|1129139\|emb\|CAA63831.1\| (X94087) similar to |
| | | | | | | | a gene located in region Xq27-28 of humans |
| | | | | | | | [Euglena gracilis] |
| 2558 | LIB3602-060-Q6-K1-D2 | 464980 | BLASTX | 436 | 5.00E−43 | 55 | UBIQUITIN-CONJUGATING ENZYME E2-17 KD |
| | | | | | | | (UBIQUITIN-PROTEIN LIGASE) |
| | | | | | | | (UBIQUITIN CARRIER PROTEIN) |
| | | | | | | | >gi\|166422\|gb\|AAA18528.1\| (L06967) ubiquitin |
| | | | | | | | carrier protein [Medicago sativa] |
| 2559 | LIB3602-040-Q6-K1-D7 | 6754506 | BLASTX | 188 | 7.00E−14 | 35 | like-glycosyltransferase |
| | | | | | | | >gi\|3954978\|emb\|CAA06945.1\| (AJ006278) |
| | | | | | | | acetylglucosaminyltransferase-like protein [Mus |
| | | | | | | | musculus] |
| 2560 | LIB3602-047-Q6-K1-F9 | 7486019 | BLASTX | 227 | 2.00E−18 | 62 | hypothetical protein F23E12.190 - Arabidopsis |
| | | | | | | | thaliana >gi\|3080425\|emb\|CAA18744.1\| |
| | | | | | | | (AL022604) putative protein [Arabidopsis thaliana] |
| | | | | | | | >gi\|7270477\|emb\|CAB80242.1\| (AL161587) |
| | | | | | | | putative protein [Arabidopsis thaliana] |
| 2561 | LIB3602-076-Q6-K6-B12 | 7428834 | BLASTX | 218 | 4.00E−18 | 68 | probable P-glycoprotein pgp1 - Arabidopsis thaliana |
| | | | | | | | >gi\|3355477\|gb\|AAC27839.1\| (AC004218) putative |
| | | | | | | | ABC transporter [Arabidopsis thaliana] |
| 2562 | LIB3602-038-Q6-K1-G6 | 6580769 | BLASTX | 346 | 2.00E−32 | 67 | (AF088897) formate acetyltransferase [Zymomonas |
| | | | | | | | mobilis] |
| 2563 | LIB3602-034-Q6-K1-E5 | 7020000 | BLASTX | 169 | 1.00E−11 | 41 | (AK000120) unnamed protein product [Homo |
| | | | | | | | sapiens] |
| 2564 | LIB3602-059-Q6-K1-A3 | 421929 | BLASTX | 568 | 1.00E−58 | 85 | polyubiquitin - tomato |
| | | | | | | | >gi\|312160\|emb\|CAA51679.1\| (X73156) ubiquitin |
| | | | | | | | [Lycopersicon esculentum] |
| 2565 | LIB3602-092-Q6-K6-B3 | 296593 | BLASTN | 260 | 1.00E−144 | 94 | H. vulgare pZE40 gene |
| 2566 | LIB3602-063-Q1-K6-E5 | 3450955 | BLASTX | 267 | 2.00E−23 | 72 | (AF020736) ATPase homolog [Homo sapiens] |
| 2567 | LIB3602-048-Q6-K1QA-D7 | 2341033 | BLASTX | 451 | 1.00E−44 | 55 | (AC000104) Similar to Babesia aldo-keto reductase |
| | | | | | | | (gb\|M93122). [Arabidopsis thaliana] |
| 2568 | LIB3602-023-Q6-K1-A8 | 7488984 | BLASTX | 163 | 6.00E−11 | 39 | dem protein - tomato |
| | | | | | | | >gi\|2190419\|emb\|CAA73973.1\| (Y13632) dem |
| | | | | | | | [Lycopersicon esculentum]. |
| 2569 | LIB3602-011-Q6-K1-D8 | 3024430 | BLASTX | 321 | 2.00E−29 | 43 | PYRROLINE-5-CARBOXYLATE REDUCTASE |
| | | | | | | | (P5CR) (P5C REDUCTASE) |
| | | | | | | | >gi\|1928962\|gb\|AAC14482.1\| (U92287) pyrroline- |
| | | | | | | | 5-carboxylate reductase [Actinidia deliciosa] |
| 2570 | LIB3602-101-Q1-K1-B3 | 1351139 | BLASTX | 224 | 4.00E−35 | 51 | SUCROSE SYNTHASE ISOFORM I (SUCROSE- |
| | | | | | | | UDP GLUCOSYLTRANSFERASE 1) |
| | | | | | | | (SUSY*DC1) >gi\|480971\|pir\|\|S37560 sucrose |
| | | | | | | | synthase (EC 2.4.1.13) - carrot |
| | | | | | | | >gi\|406317\|emb\|CAA53081.1\| (X75332) sucrose |
| | | | | | | | synthase [Daucus carota] |
| | | | | | | | >gi\|2760539\|emb\|CAA76056.1\| (Y16090) sucrose |
| | | | | | | | synt |
| 2571 | LIB3602-111-Q1-K1-G11 | 2493127 | BLASTX | 860 | 1.00E−92 | 96 | VACUOLAR ATP SYNTHASE SUBUNIT B |
| | | | | | | | ISOFORM 1 (V-ATPASE B SUBUNIT) |
| | | | | | | | >gi\|1303677\|dbj\|BAA09099.1\| (D50530) adenosine |
| | | | | | | | triphosphatase B subunit [Acetabularia acetabulum] |
| 2572 | LIB3602-011-Q6-K1-D3 | 4508082 | BLASTX | 329 | 2.00E−30 | 83 | (AC005882) Putative RNA polymerase II subunit |
| | | | | | | | Rpb10 [Arabidopsis thaliana] |
| 2573 | LIB3602-072-Q1-K1-D12 | 3182922 | BLASTX | 615 | 7.00E−64 | 72 | ADP-RIBOSYLATION FACTOR 1 |
| | | | | | | | >gi\|1654142\|gb\|AAB17725.1\| (U38470) small GTP- |
| | | | | | | | binding protein ARF [Brassica rapa] |
| 2574 | LIB3602-071-Q1-K1-D10 | 6503190 | BLASTX | 184 | 2.00E−13 | 40 | (AF200688) tyrosine kinase ZAK1 [Dictyostelium |
| | | | | | | | discoideum] |
| 2575 | LIB3602-016-Q6-K1-B11 | 4309741 | BLASTX | 233 | 3.00E−19 | 38 | (AC006439) hypothetical protein [Arabidopsis |
| | | | | | | | thaliana] |
| 2576 | LIB3602-053-Q6-K1-D10 | 1076202 | BLASTX | 217 | 3.00E−17 | 44 | calcium-stimulated protein kinase - |
| | | | | | | | Chlamydomonas eugametos |
| | | | | | | | >gi\|806542\|emb\|CAA89202.1\| (Z49233) calcium- |
| | | | | | | | stimulated protein kinase [Chlamydomonas |
| | | | | | | | eugametos] |
| 2577 | LIB3602-113-Q1-K1-A10 | 1071912 | BLASTX | 410 | 3.00E−51 | 49 | cysteine synthase (EC 4.2.99.8) cpACS1 - |
| | | | | | | | Arabidopsis thaliana >gi\|572517\|emb\|CAA57344.1\| |
| | | | | | | | (X81698) cysteine synthase [Arabidopsis thaliana] |
| 2578 | LIB3602-054-Q6-K1-A7 | 82056 | BLASTX | 258 | 4.00E−22 | 36 | protein kinase, calcium-dependent (EC 2.7.1.—) - |
| | | | | | | | carrot (fragment) |
| 2579 | LIB3602-109-Q1-K1-H10 | 7440340 | BLASTX | 415 | 4.00E−43 | 79 | ribosomal protein S14, cytosolic - Arabidopsis |
| | | | | | | | thaliana >gi\|4886269\|emb\|CAB43407.1\| |
| | | | | | | | (AL050300) putative ribosomal protein S14 |
| | | | | | | | [Arabidopsis thaliana] |
| 2580 | LIB3602-005-Q1-K1-D5 | 2854202 | BLASTX | 243 | 2.00E−20 | 35 | (AF045646) contains similarity to enoly-CoA |
| | | | | | | | hydratases [Caenorhabditis elegans] |
| 2581 | LIB3602-086-Q6-K1-F10 | 2854203 | BLASTX | 197 | 7.00E−15 | 40 | (AF045646) contains similarity to the human |
| | | | | | | | polyposis locus-encoded protein (SW: Q00765) |
| | | | | | | | [Caenorhabditis elegans] |
| 2582 | LIB3602-053-Q6-K1-C2 | 99600 | BLASTX | 301 | 4.00E−27 | 78 | chlorophyll a/b-binding protein - upland cotton |
| 2583 | LIB3602-029-Q6-K1-A11 | 462079 | BLASTX | 393 | 7.00E−38 | 64 | FERREDOXIN PRECURSOR |
| | | | | | | | >gi\|7427604\|pir\|\|FEKM ferredoxin [2Fe—2S] |
| | | | | | | | precursor, chloroplast - Chlamydomonas reinhardtii |
| | | | | | | | >gi\|409202\|gb\|AAA33085.1\| (L10349) ferredoxin |
| | | | | | | | [Chlamydomonas reinhardtii] |
| | | | | | | | >gi\|1009714\|gb\|AAC49171.1\| (U29516) ferredoxin |
| | | | | | | | precursor [Chlamydom |
| 2584 | LIB3602-032-Q6-K1-A11 | 7488014 | BLASTX | 296 | 1.00E−26 | 63 | photosystem II protein W homolog T5F17.110 - |
| | | | | | | | Arabidopsis thaliana |
| | | | | | | | >gi\|7269721\|emb\|CAB81454.1\| (AL161573) |
| | | | | | | | photosystem II protein W-like [Arabidopsis |
| | | | | | | | thaliana] |
| 2585 | LIB3602-060-Q6-K1-H12 | 5729913 | BLASTX | 316 | 6.00E−29 | 49 | mannosidase, alpha, class 1A, member 2 |
| | | | | | | | >gi\|3127047\|gb\|AAC26169.1\| (AF027156) alpha |
| | | | | | | | 1,2-mannosidase IB [Homo sapiens] |
| 2586 | LIB3602-074-Q1-K1-A7 | 3182986 | BLASTX | 222 | 8.00E−18 | 34 | PUTATIVE TRANSLATION INITIATION |
| | | | | | | | FACTOR EIF-2B SUBUNIT 2 (EIF-2B GDP-GTP |
| | | | | | | | EXCHANGE FACTOR) >gi\|7447866\|pir\|\|D69504 |
| | | | | | | | translation initiation factor eIF-2B, subunit delta |
| | | | | | | | (eif2BD) homolog - Archaeoglobus fulgidus |
| | | | | | | | >gi\|2648498\|gb\|AAB89217.1\| (AE000962) |
| | | | | | | | translatio |
| 2587 | LIB3602-085-Q6-K1-C7 | 6646754 | BLASTX | 446 | 5.00E−44 | 50 | (AC013258) unknown protein [Arabidopsis |
| | | | | | | | thaliana] |
| 2588 | LIB3602-051-Q6-K1-E4 | 7486079 | BLASTX | 317 | 3.00E−29 | 56 | hypothetical protein F24J7.100 - Arabidopsis |
| | | | | | | | thaliana >gi\|2853081\|emb\|CAA16931.1\| |
| | | | | | | | (AL021768) ATP binding protein-like [Arabidopsis |
| | | | | | | | thaliana] >gi\|7268750\|emb\|CAB78956.1\| |
| | | | | | | | (AL161551) ATP binding protein-like [Arabidopsis |
| | | | | | | | thaliana] |
| 2589 | LIB3602-014-Q6-K1-A10 | 4585874 | BLASTX | 487 | 3.00E−49 | 75 | (AC005850) 60s ribosomal protein L3 [Arabidopsis |
| | | | | | | | thaliana] |
| 2590 | LIB3602-102-Q1-K1-H7 | 5669169 | BLASTX | 150 | 1.00E−16 | 75 | (AF157017) photosystem I reaction center subunit |
| | | | | | | | V precursor [Tortula ruralis] |
| 2591 | LIB3602-014-Q6-K1-C2 | 5903045 | BLASTX | 550 | 3.00E−56 | 67 | (AC008016) Similar to gb\|AF108945 signal |
| | | | | | | | peptidase 18 kDa subunit from Homo sapiens. |
| | | | | | | | ESTs gb\|H76629, gb\|H76949 and gb\|H76216 come |
| | | | | | | | from this gene. [Arabidopsis thaliana] |
| 2592 | LIB3602-102-Q1-K1-G5 | 7459514 | BLASTX | 246 | 1.00E−20 | 37 | conserved hypothetical protein aq_1088 - Aquifex |
| | | | | | | | aeolicus >gi\|2983568\|gb\|AAC07141.1\| (AE000722) |
| | | | | | | | hypothetical protein [Aquifex aeolicus] |
| 2593 | LIB3602-055-Q6-K1-H1 | 7529742 | BLASTX | 246 | 1.00E−20 | 61 | (AL163527) 3-isopropylmalate dehydratase-like |
| | | | | | | | protein (small subunit) [Arabidopsis thaliana] |
| 2594 | LIB3602-001-P1-K6-D2 | 4886307 | BLASTX | 816 | 2.00E−87 | 84 | (AJ242588) 1-deoxy-d-xylulose-5-phosphate |
| | | | | | | | reductoisomerase [Arabidopsis thaliana] |
| 2595 | LIB3602-030-Q6-K1-B2 | 7492644 | BLASTX | 178 | 9.00E−13 | 29 | probable helicase - fission yeast |
| | | | | | | | (Schizosaccharomyces pombe) |
| | | | | | | | >gi\|3116120\|emb\|CAA18870.1\| (AL023287) SNF2 |
| | | | | | | | family dna repair protein by similarity |
| | | | | | | | [Schizosaccharomyces pombe] |
| 2596 | LIB3602-023-Q6-K1-G3 | 7487137 | BLASTX | 220 | 1.00E−17 | 67 | hypothetical protein T18B16.20 - Arabidopsis |
| | | | | | | | thaliana >gi\|2832633\|emb\|CAA16762.1\| |
| | | | | | | | (AL021711) putative protein [Arabidopsis thaliana] |
| | | | | | | | >gi\|7268700\|emb\|CAB78907.1\| (AL161550) |
| | | | | | | | putative protein [Arabidopsis thaliana] |
| 2597 | LIB3602-092-Q6-K6-G7 | 7447935 | BLASTX | 756 | 2.00E−80 | 86 | probable potassium channel beta chain - rice |
| | | | | | | | >gi\|1197587\|gb\|AAC50046.1\| (U46758) potassium |
| | | | | | | | channel beta subunit protein [Oryza sativa] |
| 2598 | LIB3602-049-Q6-K1-F7 | 3928891 | BLASTX | 415 | 1.00E−40 | 72 | (AF093617) chlorophyll a/b binding protein |
| | | | | | | | [Acetabularia acetabulum] |
| 2599 | LIB3602-040-Q6-K1-G11 | 7484622 | BLASTX | 525 | 2.00E−53 | 74 | L-ascorbate peroxidase (EC 1.11.1.11) precursor, |
| | | | | | | | stromal - common ice plant |
| | | | | | | | >gi\|3202026\|gb\|AAC19394.1\| (AF069316) stromal |
| | | | | | | | L-ascorbate peroxidase precursor |
| | | | | | | | [Mesembryanthemum crystallinum] |
| 2600 | LIB3602-064-Q1-K6-A8 | 5903046 | BLASTX | 243 | 3.00E−20 | 64 | (AC008016) F6D8.19 [Arabidopsis thaliana] |
| 2601 | LIB3602-025-Q6-K1-D12 | 1402884 | BLASTX | 660 | 3.00E−69 | 72 | (X98130) unknown [Arabidopsis thaliana] |
| | | | | | | | >gi\|1495265\|emb\|CAA66120.1\| (X97488) beta- |
| | | | | | | | transducin like protein [Arabidopsis thaliana] |
| 2602 | LIB3602-053-Q6-K1-F10 | 7486400 | BLASTX | 324 | 5.00E−30 | 55 | hypothetical protein F4B14.80 - Arabidopsis |
| | | | | | | | thaliana >gi\|3805847\|emb\|CAA21467.1\| |
| | | | | | | | (AL031986) putative protein [Arabidopsis thaliana] |
| | | | | | | | >gi\|7270533\|emb\|CAB81490.1\| (AL161588) |
| | | | | | | | putative protein [Arabidopsis thaliana] |
| 2603 | LIB3602-094-Q6-K6-E7 | 131225 | BLASTX | 210 | 2.00E−16 | 40 | PHOTOSYSTEM I REACTION CENTRE |
| | | | | | | | SUBUNIT XI PRECURSOR (SUBUNIT V) (PSI- |
| | | | | | | | L) >gi\|100605\|pir\|\|A39759 photosystem I 18K |
| | | | | | | | protein precursor - barley |
| | | | | | | | >gi\|167087\|gb\|AAA62700.1\| (M61146) |
| | | | | | | | photosystem I hydrophobic protein [Hordeum |
| | | | | | | | vulgare] |
| 2604 | LIB3602-022-Q6-K1-G2 | 7493530 | BLASTX | 254 | 1.00E−21 | 43 | trp-asp repeat protein - fission yeast |
| | | | | | | | (Schizosaccharomyces pombe) |
| | | | | | | | >gi\|5706505\|emb\|CAB52267.1\| (AL109739) trp-asp |
| | | | | | | | repeat protein [Schizosaccharomyces pombe] |
| 2605 | LIB3602-057-Q6-K1-G1 | 6094048 | BLASTX | 226 | 7.00E−36 | 75 | 60S RIBOSOMAL PROTEIN L30 |
| | | | | | | | >gi\|2879811\|emb\|CAA11256.1\| (AJ223316) |
| | | | | | | | ribosomal protein L30 [Lupinus luteus] |
| 2606 | LIB3602-027-Q6-K1-C3 | 4193382 | BLASTX | 311 | 3.00E−28 | 86 | (AF083336) ribosomal protein S27 [Arabidopsis |
| | | | | | | | thaliana] >gi\|4193384\|gb\|AAD10030.1\| (AF083337) |
| | | | | | | | ribosomal protein S27 [Arabidopsis thaliana] |
| 2607 | LIB3602-006-Q1-K1-B8 | 6319146 | BLASTX | 219 | 2.00E−17 | 89 | (AF193345) H2A protein [Oryza sativa] |
| 2608 | LIB3602-103-Q1-K1-D3 | 1708781 | BLASTX | 643 | 3.00E−67 | 73 | (X97729) LMP7-like protein [Botryllus schlosseri] |
| 2609 | LIB3602-013-Q6-K1-H9 | 7295382 | BLASTX | 148 | 2.00E−09 | 54 | (AE003564) CG10467 gene product [Drosophila |
| | | | | | | | melanogaster] |
| 2610 | LIB3602-054-Q6-K1-D1 | 6850936 | BLASTX | 169 | 7.00E−12 | 80 | (AJ271667) putative proteasome regulatory subunit |
| | | | | | | | [Cicer arietinum] |
| 2611 | LIB3602-044-Q6-K1-E12 | 7294816 | BLASTX | 540 | 3.00E−55 | 67 | (AE003547) hay gene product [Drosophila |
| | | | | | | | melanogaster] |
| 2612 | LIB3602-069-Q1-K1-B7 | 7529717 | BLASTX | 706 | 2.00E−74 | 72 | (AL132969) fructose bisphosphate aldolase-like |
| | | | | | | | protein [Arabidopsis thaliana] |
| 2613 | LIB3602-005-Q1-K1-F2 | 7384808 | BLASTX | 335 | 5.00E−31 | 57 | (AB040503) cysteine synthase [Allium tuberosum] |
| 2614 | LIB3602-042-Q6-K1-C6 | 7296277 | BLASTX | 200 | 3.00E−15 | 36 | (AE003590) CG2761 gene product [Drosophila |
| | | | | | | | melanogaster] |
| 2615 | LIB3602-006-Q1-K1-D9 | 128825 | BLASTX | 415 | 2.00E−40 | 51 | NADH-UBIQUINONE OXIDOREDUCTASE 75 KD |
| | | | | | | | SUBUNIT PRECURSOR (COMPLEX I- |
| | | | | | | | 75 KD) (CI-75 KD) >gi\|108830\|pir\|\|A33552 NADH |
| | | | | | | | dehydrogenase (ubiquinone) (EC 1.6.5.3) 75K chain |
| | | | | | | | precursor - bovine >gi\|163414\|gb\|AAA30662.1\| |
| | | | | | | | (J02877) NADH: ubiquinone reductase precursor |
| | | | | | | | [Bos |
| 2616 | LIB3602-014-Q6-K1-F10 | 7025849 | BLASTX | 166 | 2.00E−11 | 38 | AC024128) hypothetical protein [Arabidopsis |
| | | | | | | | thaliana] |
| 2617 | LIB3602-066-Q1-K6-F7 | 7493333 | BLASTX | 308 | 5.00E−28 | 50 | rna binding protein - fission yeast |
| | | | | | | | (Schizosaccharomyces pombe) |
| | | | | | | | >gi\|3184100\|emb\|CAA19313.1\| (AL023777) |
| | | | | | | | putative RNA-binding protein |
| | | | | | | | [Schizosaccharomyces pombe] |
| 2618 | LIB3602-064-Q1-K6-B4 | 6721114 | BLASTX | 341 | 8.00E−32 | 59 | (AC007396) T4O12.18 [Arabidopsis thaliana] |
| 2619 | LIB3602-049-Q6-K1-E9 | 4102582 | BLASTX | 198 | 4.00E−15 | 38 | (AF013115) CAO [Arabidopsis thaliana] |
| 2620 | LIB3602-014-Q6-K1-E11 | 2501296 | BLASTX | 211 | 1.00E−16 | 61 | DNA GYRASE SUBUNIT B |
| | | | | | | | >gi\|7469301\|pir\|\|S77162 DNA topoisomerase (ATP- |
| | | | | | | | hydrolyzing) (EC 5.99.1.3) chain B - Synechocystis |
| | | | | | | | sp. (strain PCC 6803) |
| | | | | | | | >gi\|1652801\|dbj\|BAA17720.1\| (D90908) DNA |
| | | | | | | | gyrase B subunit [Synechocystis sp.] |
| 2621 | LIB3602-058-Q6-K1-A3 | 2088652 | BLASTX | 281 | 8.00E−25 | 37 | (AF002109) 26S proteasome regulatory subunit |
| | | | | | | | [Arabidopsis thaliana] |
| | | | | | | | >gi\|2351376\|gb\|AAD03463.1\| (U54561) translation |
| | | | | | | | initiation factor eIF2 p47 subunit homolog |
| | | | | | | | [Arabidopsis thaliana] |
| 2622 | LIB3602-073-Q1-K1-B5 | 3915037 | BLASTX | 355 | 2.00E−33 | 59 | SUCROSE SYNTHASE 2 (SUCROSE-UDP |
| | | | | | | | GLUCOSYLTRANSFERASE 2) |
| | | | | | | | >gi\|7433834\|pir\|\|T06497 probable sucrose synthase |
| | | | | | | | (EC 2.4.1.13) 2 - garden pea |
| | | | | | | | >gi\|2570067\|emb\|CAA04512.1\| (AJ001071) second |
| | | | | | | | sucrose synthase [Pisum sativum] |
| 2623 | LIB3602-012-Q6-K1-D2 | 6225722 | BLASTX | 202 | 2.00E−15 | 36 | MRP PROTEIN HOMOLOG |
| | | | | | | | >gi\|7446831\|pir\|\|G70364 conserved hypothetical |
| | | | | | | | protein aq_737 - Aquifex aeolicus |
| | | | | | | | >gi\|2983325\|gb\|AAC06915.1\| (AE000705) |
| | | | | | | | hypothetical protein [Aquifex aeolicus] |
| 2624 | LIB3602-059-Q6-K1-H6 | 7440317 | BLASTX | 166 | 2.00E−17 | 66 | ribosomal protein S14 - mouse |
| 2625 | LIB3602-022-Q6-K1-C3 | 6358788 | BLASTX | 449 | 2.00E−44 | 54 | (AC010852) unknown protein [Arabidopsis |
| | | | | | | | thaliana] |
| 2626 | LIB3602-021-Q6-K1-C8 | 4581114 | BLASTX | 349 | 3.00E−33 | 69 | (AC005825) putative HesB-like protein |
| | | | | | | | [Arabidopsis thaliana] |
| 2627 | LIB3602-018-Q6-K1-A3 | 3914161 | BLASTX | 175 | 2.00E−12 | 46 | CYTOCHROME P450 55A2 (CYTOCHROME |
| | | | | | | | P450NOR1) >gi\|1107453\|dbj\|BAA11408.1\| |
| | | | | | | | (D78511) cytochrome P450nor1 [Cylindrocarpon |
| | | | | | | | lichenicola] |
| 2628 | LIB3602-092-Q6-K6-H5 | 2492612 | BLASTX | 399 | 2.00E−38 | 73 | 1-AMINOCYCLOPROPANE-1-CARBOXYLATE |
| | | | | | | | OXIDASE (ACC OXIDASE) (ETHYLENE- |
| | | | | | | | FORMING ENZYME) (EFE) |
| | | | | | | | >gi\|1076734\|pir\|\|S52712 1-aminocyclopropane-1- |
| | | | | | | | carboxylate oxidase (ACC oxidase) - rice |
| | | | | | | | >gi\|755773\|emb\|CAA59749.1\| (X85747) 1- |
| | | | | | | | aminocyclopropane-1-carboxylate oxidase (A |
| 2629 | LIB3602-079-Q6-K6-B8 | 7025861 | BLASTX | 226 | 3.00E−18 | 29 | (AC024128) putative transporter [Arabidopsis |
| | | | | | | | thaliana] |
| 2630 | LIB3602-064-Q1-K6-G4 | 3913437 | BLASTX | 279 | 2.00E−24 | 75 | PUTATIVE PRE-MRNA SPLICING FACTOR |
| | | | | | | | ATP-DEPENDENT RNA HELICASE |
| | | | | | | | >gi\|1402875\|emb\|CAA66825.1\| (X98130) RNA |
| | | | | | | | helicase [Arabidopsis thaliana] |
| | | | | | | | >gi\|1495271\|emb\|CAA66613.1\| (X97970) RNA |
| | | | | | | | helicase [Arabidopsis thaliana] |
| 2631 | LIB3602-071-Q1-K1-C12 | 3264790 | BLASTX | 241 | 5.00E−20 | 85 | (AF070942) ribulose-phosphate 3-epimerase transit |
| | | | | | | | form [Expression vector pFL505] |
| 2632 | LIB3602-120-Q1-K1-C12 | 6225908 | BLASTX | 144 | 4.00E−09 | 81 | 60S RIBOSOMAL PROTEIN L37A |
| | | | | | | | >gi\|7441205\|pir\|\|C71614 ribosomal protein L37A |
| | | | | | | | PFB0455w - malaria parasite (Plasmodium |
| | | | | | | | falciparum) >gi\|3845189\|gb\|AAC71880.1\| |
| | | | | | | | (AE001396) ribosomal protein L37A [Plasmodium |
| | | | | | | | falciparum] |
| 2633 | LIB3602-018-Q6-K1-F12 | 5114047 | BLASTX | 351 | 4.00E−33 | 48 | (AF085356) putative RNA helicase [Homo sapiens] |
| 2634 | LIB3602-079-Q6-K6-C6 | 2144171 | BLASTX | 156 | 5.00E−10 | 40 | capreomycin acetyltransferase (EC 2.3.1.—) - |
| | | | | | | | Streptomyces capreolus |
| | | | | | | | >gi\|1223838\|gb\|AAA92035.1\| (U13077) |
| | | | | | | | capreomycin acetyltransferase [Streptomyces |
| | | | | | | | capreolus] >gi\|1586532\|prf\|\|2204233A capreomycin |
| | | | | | | | acetyltransferase [Streptomyces capreolus] |
| 2635 | LIB3602-089-Q6-K6-G4 | 2960216 | BLASTX | 395 | 3.00E−44 | 66 | (AJ223384) 26S proteasome regulatory ATPase |
| | | | | | | | subunit 10b (S10b) [Manduca sexta] |
| 2636 | LIB3602-114-Q1-K1-F5 | 7486220 | BLASTX | 258 | 5.00E−22 | 43 | hypothetical protein F27B13.100 - Arabidopsis |
| | | | | | | | thaliana >gi\|4914410\|emb\|CAB43661.1\| |
| | | | | | | | (AL050352) putative protein [Arabidopsis thaliana] |
| | | | | | | | >gi\|7269885\|emb\|CAB79744.1\| (AL161575) |
| | | | | | | | putative protein [Arabidopsis thaliana] |
| 2637 | LIB3602-045-Q6-K1-B4 | 4758714 | BLASTX | 243 | 2.00E−20 | 40 | microsomal glutathione S-transferase 3 |
| | | | | | | | >gi\|7387731\|sp\|O14880\|GST3_HUMAN |
| | | | | | | | MICROSOMAL GLUTATHIONE S- |
| | | | | | | | TRANSFERASE 3 (MICROSOMAL GST-3) |
| | | | | | | | (MICROSOMAL GST-III) |
| | | | | | | | >gi\|2583081\|gb\|AAB82609.1\| (AF026977) |
| | | | | | | | microsomal glutathione S-transferase 3 [Homo |
| | | | | | | | sapiens] |
| 2638 | LIB3602-017-Q6-K1-H9 | 4206112 | BLASTX | 208 | 1.00E−16 | 100 | (AF097662) alpha tubulin [Mesembryanthemum |
| | | | | | | | crystallinum] |
| 2639 | LIB3602-078-Q6-K6-E2 | 4586592 | BLASTX | 687 | 3.00E−72 | 68 | (AB025000) multicatalytic endopeptidase complex |
| | | | | | | | [Cicer arietinum] |
| 2640 | LIB3602-119-Q1-K1-E8 | 7296284 | BLASTX | 255 | 1.00E−37 | 77 | (AE003591) CG5605 gene product [alt 1] |
| | | | | | | | [Drosophila melanogaster] |
| | | | | | | | >gi\|7296285\|gb\|AAF51575.1\| (AE003591) CG5605 |
| | | | | | | | gene product [alt 2] [Drosophila melanogaster] |
| 2641 | LIB3602-120-Q1-K1-B3 | 100596 | BLASTX | 157 | 3.00E−10 | 100 | metallothionein-like protein - barley |
| | | | | | | | >gi\|234815\|gb\|AAB19699.1\| (S53707) B22EL8 |
| | | | | | | | [Hordeum vulgare = barley, aleurone cells, Peptide, |
| | | | | | | | 115 aa] >gi\|228182\|prf\|\|1718305A B22E protein |
| | | | | | | | [Hordeum vulgare] |
| 2642 | LIB3602-014-Q6-K1-G9 | 7543911 | BLASTX | 477 | 6.00E−48 | 70 | (AL163572) ubiquinol- cytochrome-c reductase- |
| | | | | | | | like protein [Arabidopsis thaliana] |
| 2643 | LIB3602-078-Q6-K6-A7 | 7505775 | BLASTX | 226 | 8.00E−19 | 81 | hypothetical protein K11D2.3 - Caenorhabditis |
| | | | | | | | elegans |
| 2644 | LIB3602-105-Q1-K1-A8 | 117519 | BLASTX | 642 | 6.00E−70 | 76 | PHYTOENE DEHYDROGENASE PRECURSOR |
| | | | | | | | (PHYTOENE DESATURASE) |
| | | | | | | | >gi\|99945\|pir\|\|A39597 phytoene dehydrogenase (EC |
| | | | | | | | 1.3.—.—) - soybean >gi\|170044\|gb\|AAA34001.1\| |
| | | | | | | | (M64704) phytoene desaturase [Glycine max] |
| 2645 | LIB3602-041-Q6-K1-F4 | 6598624 | BLASTX | 189 | 3.00E−14 | 68 | (AC006837) putative ubiquinol-cytochrome c |
| | | | | | | | reductase [Arabidopsis thaliana] |
| 2646 | LIB3602-102-Q1-K1-A10 | 5679842 | BLASTX | 206 | 5.00E−16 | 51 | (AJ243961) 11332.6 [Oryza sativa] |
| 2647 | LIB3602-052-Q6-K1-F8 | 3024020 | BLASTX | 495 | 6.00E−50 | 75 | INITIATION FACTOR 5A-3 (EIF-5A) (EIF-4D) |
| | | | | | | | >gi\|7443511\|pir\|\|T07133 translation initiation factor |
| | | | | | | | eIF-5A3 - potato >gi\|2225881\|dbj\|BAA20877.1\| |
| | | | | | | | (AB004824) eukaryotic initiation factor 5A3 |
| | | | | | | | [Solanum tuberosum] |
| 2648 | LIB3602-076-Q6-K6-A6 | 1213515 | BLASTN | 38 | 2.00E−11 | 89 | Gadus morhua ribosomal protein L22 mRNA, |
| | | | | | | | partial cds |
| 2649 | LIB3602-044-Q6-K1-F11 | 1708311 | BLASTX | 681 | 9.00E−72 | 83 | CHLOROPLAST STROMA 70 KD HEAT |
| | | | | | | | SHOCK-RELATED PROTEIN |
| | | | | | | | >gi\|7441859\|pir\|\|T09119 dnaK-type molecular |
| | | | | | | | chaperone HSP80 precursor, chloroplast - spinach |
| | | | | | | | (fragment) >gi\|170094\|gb\|AAA18570.1\| (M99565) |
| | | | | | | | 80 kDa heat shock protein [Spinacia oleracea] |
| 2650 | LIB3602-004-Q1-K1-C9 | 6069644 | BLASTX | 157 | 2.00E−10 | 35 | (AP000616) similar to Bacillus subtilis genome; |
| | | | | | | | unknown protein (Z99110) [Oryza sativa] |
| 2651 | LIB3602-047-Q6-K1-H9 | 7302323 | BLASTX | 419 | 5.00E−41 | 59 | (AE003790) CG3358 gene product [Drosophila |
| | | | | | | | melanogaster] |
| 2652 | LIB3602-062-Q6-K1-D3 | 7267302 | BLASTX | 510 | 1.00E−51 | 61 | (AL161503) UV-damaged DNA binding factor-like |
| | | | | | | | protein [Arabidopsis thaliana] |
| 2653 | LIB3602-104-Q1-K1-G11 | 1363492 | BLASTX | 353 | 4.00E−33 | 42 | outer envelope membrane protein OEP75 precursor - |
| | | | | | | | garden pea >gi\|576507\|gb\|AAA53275.1\| (L36858) |
| | | | | | | | outer membrane protein [Pisum sativum] |
| | | | | | | | >gi\|633607\|emb\|CAA58720.1\| (X83767) |
| | | | | | | | chloroplastic outer envelope membrane protein |
| | | | | | | | (OEP75) [Pisum sativum] |
| 2654 | LIB3602-019-Q6-K1-B2 | 7435702 | BLASTX | 243 | 1.00E−20 | 50 | hypothetical protein - Synechocystis sp. (strain PCC |
| | | | | | | | 6803) >gi\|1653655\|dbj\|BAA18567.1\| (D90915) |
| | | | | | | | ATP-dependent Clp protease proteolytic subunit |
| | | | | | | | [Synechocystis sp.] |
| 2655 | LIB3602-003-Q1-K1-G1 | 2982322 | BLASTX | 471 | 5.00E−47 | 66 | (AF051246) probable proteasome subunit [Picea |
| | | | | | | | mariana] |
| 2656 | LIB3602-079-Q6-K6-F12 | 1345838 | BLASTX | 774 | 2.00E−82 | 75 | PHYTOENE DEHYDROGENASE PRECURSOR |
| | | | | | | | (PHYTOENE DESATURASE) |
| | | | | | | | >gi\|2130143\|pir\|S65060 phytoene desaturase |
| | | | | | | | precursor - maize >gi\|1051180\|gb\|AAC12846.1\| |
| | | | | | | | (U37285) phytoene desaturase [Zea mays] |
| 2657 | LIB3602-005-Q1-K1-A6 | 5921735 | BLASTX | 305 | 2.00E−27 | 57 | 10 KD CHAPERONIN (PROTEIN CPN10) |
| | | | | | | | (PROTEIN GROES) >gi\|1519241\|gb\|AAB07452.1\| |
| | | | | | | | (U65890) 10 kDa chaperonin [Brassica napus] |
| 2658 | LIB3602-029-Q6-K1-G3 | 6831610 | BLASTX | 377 | 5.00E−36 | 57 | 60S RIBOSOMAL PROTEIN L13 (BBC1 |
| | | | | | | | PROTEIN HOMOLOG) |
| | | | | | | | >gi\|2662188\|dbj\|BAA23724.1\| (AB009086) BBC1 |
| | | | | | | | protein [Chlamydomonas sp.] |
| 2659 | LIB3602-008-Q6-K1-A4 | 4584358 | BLASTX | 198 | 5.00E−15 | 45 | (AC006420) unknown protein [Arabidopsis |
| | | | | | | | thaliana] |
| 2660 | LIB3602-029-Q6-K1-A9 | 4557467 | BLASTX | 167 | 2.00E−11 | 35 | Cockayne syndrome 1 protein |
| | | | | | | | >gi\|3121917\|sp\|Q13216\|CSA_HUMAN |
| | | | | | | | COCKAYNE SYNDROME WD-REPEAT |
| | | | | | | | PROTEIN CSA >gi\|1362773\|pir\|\|A57090 CSA |
| | | | | | | | protein - human >gi\|975302\|gb\|AAA82605.1\| |
| | | | | | | | (U28413) CSA protein [Homo sapiens] |
| 2661 | LIB3602-015-Q6-K1-C6 | 121985 | BLASTX | 342 | 7.00E−32 | 71 | HISTONE H2A-IV >gi\|99437\|pir\|\|JQ0796 histone |
| | | | | | | | H2A.IV - Volvox carteri |
| | | | | | | | >gi\|170658\|gb\|AAA34249.1\| (M31922) histone |
| | | | | | | | H2A-IV [Volvox carteri] |
| 2662 | LIB3602-087-Q6-K1-A12 | 3914899 | BLASTX | 233 | 4.00E−19 | 57 | 40S RIBOSOMAL PROTEIN S4 |
| | | | | | | | >gi\|7440142\|pir\|\|T01187 ribosomal protein S4 type |
| | | | | | | | I - maize >gi\|2331301\|gb\|AAB66899.1\| (AF013487) |
| | | | | | | | ribosomal protein S4 type I [Zea mays] |
| 2663 | LIB3602-061-Q6-K1-C10 | 266944 | BLASTX | 633 | 4.00E−66 | 70 | 60S RIBOSOMAL PROTEIN L2 (L8) |
| | | | | | | | (RIBOSOMAL PROTEIN TL2) |
| | | | | | | | >gi\|71078\|pir\|\|R5TOL8 ribosomal protein L8, |
| | | | | | | | cytosolic - tomato >gi\|19343\|emb\|CAA45863.1\| |
| | | | | | | | (X64562) ribosomal protein L2 [Lycopersicon |
| | | | | | | | esculentum] |
| 2664 | LIB3602-071-Q1-K1-B9 | 463856 | BLASTN | 59 | 3.00E−24 | 97 | Chlamydomonas reinhardtii 21gr ribosomal protein |
| | | | | | | | S14 (CRY1) gene, complete cds |
| 2665 | LIB3602-080-Q6-K6-G8 | 2500129 | BLASTX | 366 | 1.00E−34 | 72 | THIOREDOXIN REDUCTASE I (NADPH- |
| | | | | | | | DEPENDENT THIOREDOXIN REDUCTASE 1) |
| | | | | | | | (NTR 1) >gi\|468526\|emb\|CAA80656.1\| (Z23109) |
| | | | | | | | Thioredoxin reductase [Arabidopsis thaliana] |
| 2666 | LIB3602-068-Q1-K1-G1 | 7416846 | BLASTX | 190 | 5.00E−14 | 51 | (AB025969) NAD-dependent sorbitol |
| | | | | | | | dehydrogenase [Prunus persica] |
| 2667 | LIB3602-086-Q6-K1-E2 | 6633812 | BLASTX | 163 | 6.00E−11 | 36 | (AC009519) F1N19.9 [Arabidopsis thaliana] |
| 2668 | LIB3602-045-Q6-K1-H8 | 7023297 | BLASTX | 153 | 8.00E−10 | 37 | (AK001798) unnamed protein product [Homo |
| | | | | | | | sapiens] |
| 2669 | LIB3602-028-Q6-K1-F2 | 7076802 | BLASTX | 563 | 7.00E−58 | 64 | (AL132975) mannose-1-phosphate |
| | | | | | | | guanylyltransferase-like protein [Arabidopsis |
| | | | | | | | thaliana] |
| 2670 | LIB3602-022-Q6-K1-D6 | 5823571 | BLASTX | 265 | 6.00E−23 | 72 | (AL049730) Ribosomal protein L7Ae-like |
| | | | | | | | [Arabidopsis thaliana] |
| | | | | | | | >gi\|7267962\|emb\|CAB78303.1\| (AL161534) |
| | | | | | | | Ribosomal protein L7Ae-like [Arabidopsis thaliana] |
| 2671 | LIB3602-020-Q6-K1-B5 | 6093872 | BLASTX | 268 | 8.00E−24 | 73 | 60S RIBOSOMAL PROTEIN L15-2 |
| | | | | | | | >gi\|2982318\|gb\|AAC32144.1\| (AF051244) probable |
| | | | | | | | 60S ribosomal protein L15 [Picea mariana] |
| 2672 | LIB3602-073-Q1-K1-B6 | 6277254 | BLASTX | 148 | 4.00E−09 | 59 | (AB034249) geranylgeranyl pyrophosphate |
| | | | | | | | synthase [Croton sublyratus] |
| 2673 | LIB3602-035-Q1-K1-H10 | 6434960 | BLASTX | 324 | 9.00E−30 | 41 | (AF145311) 26S proteasome regulatory complex |
| | | | | | | | subunit p39A [Drosophila melanogaster] |
| | | | | | | | >gi\|7301037\|gb\|AAF56173.1\| (AE003744) Rpn4 |
| | | | | | | | gene product [alt 1] [Drosophila melanogaster] |
| | | | | | | | >gi\|7301038\|gb\|AAF56174.1\| (AE003744) Rpn4 |
| | | | | | | | gene product [alt 2] [Drosophila melan |
| 2674 | LIB3602-008-Q6-K1-D2 | 6633814 | BLASTX | 174 | 2.00E−12 | 63 | (AC009519) F1N19.11 [Arabidopsis thaliana] |
| 2675 | LIB3602-046-Q6-K1-H4 | 7486788 | BLASTX | 279 | 1.00E−24 | 59 | hypothetical protein M3E9.70 - Arabidopsis |
| | | | | | | | thaliana >gi\|2982456\|emb\|CAA18220.1\| |
| | | | | | | | (AL022223) putative protein [Arabidopsis thaliana] |
| | | | | | | | >gi\|726950\|emb\|CAB79505.1\| (AL161565) |
| | | | | | | | putative protein [Arabidopsis thaliana] |
| 2676 | LIB3602-026-Q6-K1-C5 | 4808539 | BLASTX | 185 | 2.00E−13 | 66 | (AF075700) putative glutathione synthetase; |
| | | | | | | | GSHS2 [Medicago truncatula] |
| 2677 | LIB3602-048-Q6-K1QA- | 6714389 | BLASTX | 324 | 5.00E−30 | 61 | (AC012393) hypothetical protein [Arabidopsis |
| | E12 | | | | | | thaliana] |
| 2678 | LIB3602-054-Q6-K1-H1 | 3024001 | BLASTX | 404 | 3.00E−39 | 52 | H(+)/HEXOSE COTRANSPORTER 2 |
| | | | | | | | (GALACTOSE-H+ SYMPORTER) |
| | | | | | | | >gi\|18043\|emb\|CAA47323.1\| (X66855) HUP2 |
| | | | | | | | [Chlorella kessleri] |
| 2679 | LIB3602-105-Q1-K1-E4 | 2655889 | BLASTX | 251 | 3.00E−21 | 31 | (AL009171) 62D9.b [Drosophila melanogaster] |
| 2680 | LIB3602-053-Q6-K1-B3 | 7494794 | BLASTX | 175 | 3.00E−12 | 50 | hypothetical protein B0261.4 - Caenorhabditis |
| | | | | | | | elegans >gi\|1938549\|gb\|AAB52351.1\| (U97016) |
| | | | | | | | similar to drosophila Rlc1 gene product |
| | | | | | | | (NID: g563361) and S. cerevisiae mitochondrial 60S |
| | | | | | | | ribosomal protein L4 (YML4) (NID: g459259) |
| | | | | | | | [Caenorhabditis elegans] |
| 2681 | LIB3602-032-Q6-K1-C11 | 7484997 | BLASTX | 214 | 5.00E−17 | 50 | H+-transporting ATPase (EC 3.6.1.35) 14K chain, |
| | | | | | | | vacuolar - Arabidopsis thaliana |
| | | | | | | | >gi\|3892056\|gb\|AAC78269.1\|AAC78269 |
| | | | | | | | (AC002330) putative vacuolar ATPase [Arabidopsis |
| | | | | | | | thaliana] >gi\|7269022\|emb\|CAB80755.1\| |
| | | | | | | | (AL161494) putative vacuolar ATPase [Arabidopsis |
| | | | | | | | thal |
| 2682 | LIB3602-072-Q1-K1-A4 | 2982311 | BLASTX | 561 | 2.00E−57 | 60 | (AF051240) probable ubiquitin-conjugating enzyme |
| | | | | | | | E2 [Picea mariana] |
| 2683 | LIB3602-021-Q6-K1-E2 | 7229709 | BLASTX | 300 | 2.00E−27 | 65 | (AF237624) 80S ribosomal protein L31 [Perilla |
| | | | | | | | frutescens] |
| 2684 | LIB3602-070-Q1-K1-C7 | 7239500 | BLASTX | 231 | 6.00E−19 | 54 | (AC012654) Contains similarity to the late |
| | | | | | | | embryogenesis abundant protein from Picea glauca |
| | | | | | | | gb\|L47115. ESTs gb\|AI992795 and gb\|T45309 |
| | | | | | | | come from this gene. [Arabidopsis thaliana] |
| 2685 | LIB3602-106-Q1-K1-E10 | 3776023 | BLASTX | 230 | 6.00E−31 | 42 | (AJ010473) RNA helicase [Arabidopsis thaliana] |
| 2686 | LIB3602-022-Q6-K1-F2 | 5924062 | BLASTX | 359 | 5.00E−34 | 51 | (AF158699) unknown [Burkholderia cepacia] |
| 2687 | LIB3602-102-Q1-K1-H11 | 4680713 | BLASTX | 398 | 2.00E−51 | 71 | (AF132971) CGI-37 protein [Homo sapiens] |
| | | | | | | | >gi\|5114055\|gb\|AAD40195.1\| (AF085360) |
| | | | | | | | HSPC031 [Homo sapiens] |
| 2688 | LIB3602-059-Q6-K1-H4 | 7480237 | BLASTX | 145 | 8.00E−09 | 42 | hypothetical protein SCJ4.42c - Streptomyces |
| | | | | | | | coelicolor >gi\|5738802\|emb\|CAB52976.1\| |
| | | | | | | | (AL109950) hypothetical protein [Streptomyces |
| | | | | | | | coelicolor A3(2)] |
| 2689 | LIB3602-084-Q6-K1-F11 | 7248415 | BLASTX | 157 | 4.00E−10 | 34 | (AP001389) ESTs |
| | | | | | | | AU077873(S1878), D40121(S1878) correspond to a |
| | | | | | | | region of the predicted gene. ˜Similar to Arabidopsis |
| | | | | | | | thaliana 60S ribosomal protein L13 (P41127) |
| | | | | | | | [Oryza sativa] |
| 2690 | LIB3602-020-Q6-K1-A1 | 1174745 | BLASTX | 571 | 6.00E−59 | 80 | TRIOSEPHOSPHATE ISOMERASE, |
| | | | | | | | CHLOROPLAST PRECURSOR (TIM) |
| | | | | | | | >gi\|1363523\|pir\|\|S53761 triose-phosphate isomerase |
| | | | | | | | (EC 5.3.1.1) precursor, chloroplast - rye |
| | | | | | | | >gi\|609262\|emb\|CAA83533.1\| (Z32521) |
| | | | | | | | triosephosphate isomerase [Secale cereale] |
| | | | | | | | >gi\|1095494\|prf\|\|2109226B tr |
| 2691 | LIB3602-050-Q6-K1-C9 | 2982305 | BLASTX | 339 | 1.00E−31 | 73 | (AF051237) 60S ribosomal protein L3 [Picea |
| | | | | | | | mariana] |
| 2692 | LIB3602-039-Q6-K1-G9 | 3334374 | BLASTX | 171 | 9.00E−12 | 85 | TRANSCRIPTION INITIATION FACTOR TFIID |
| | | | | | | | (TATA-BOX FACTOR) (TATA SEQUENCE- |
| | | | | | | | BINDING PROTEIN) (TBP) |
| | | | | | | | >gi\|2708495\|gb\|AAC49985.1\| (U95549) TATA- |
| | | | | | | | binding protein [Candida albicans] |
| | | | | | | | >gi\|2708497\|gb\|AAC49986.1\| (U95550) TATA- |
| | | | | | | | binding protein [Candida albicans] |
| 2693 | LIB3602-045-Q6-K1-G11 | 4567249 | BLASTX | 251 | 3.00E−21 | 45 | (AC007070) hypothetical protein [Arabidopsis |
| | | | | | | | thaliana] |
| 2694 | LIB3602-002-P1-K6-E4 | 6174939 | BLASTX | 191 | 6.00E−15 | 81 | 60S RIBOSOMAL PROTEIN L37-B (YL27) |
| | | | | | | | >gi\|2275274\|gb\|AAB63862.1\| (U97370) 60S |
| | | | | | | | ribosomal protein homolog [Schizosaccharomyces |
| | | | | | | | pombe] |
| 2695 | LIB3602-101-Q1-K1-C6 | 121966 | BLASTX | 265 | 7.00E−23 | 69 | HISTONE H2A.1 >gi\|70689\|pir\|\|HSXLA1 histone |
| | | | | | | | H2A.1 - African clawed frog |
| | | | | | | | >gi\|64777\|emb\|CAA26817.1\| (X03018) histone |
| | | | | | | | H2A (aa 1-130) [Xenopus laevis] |
| | | | | | | | >gi\|214284\|gb\|AAA49769.1\| (M21287) histone |
| | | | | | | | H2A [Xenopus lsevis] |
| 2696 | LIB3602-013-Q6-K1-E3 | 6919944 | BLASTX | 477 | 1.00E−47 | 61 | PROBABLE 26S PROTEASOME REGULATORY |
| | | | | | | | SUBUNIT S12 (MOV34 PROTEIN) |
| | | | | | | | >gi\|2351374\|gb\|AAD03464.1\| (U54560) putative |
| | | | | | | | 26S proteasome subunit athMOV34 [Arabidopsis |
| | | | | | | | thaliana] |
| 2697 | LIB3602-073-Q1-K1-F12 | 7469855 | BLASTX | 258 | 5.00E−22 | 37 | hypothetical protein - Synechocystis sp. (strain PCC |
| | | | | | | | 6803) >gi\|1652591\|dbj\|BAA17512.1\| (D90906) |
| | | | | | | | chloroplast import-associated channel IAP75 |
| | | | | | | | [Synechocystis sp.] |
| 2698 | LIB3602-005-Q1-K1-A3 | 7486947 | BLASTX | 140 | 8.00E−09 | 64 | hypothetical protein T13E15.7 - Arabidopsis |
| | | | | | | | thaliana >gi\|2344892\|gb\|AAC31832.1\| (AC002388) |
| | | | | | | | unknown protein [Arabidopsis thaliana] |
| 2699 | LIB3602-062-Q6-K1-E2 | 7487839 | BLASTX | 573 | 5.00E−59 | 63 | hypothetical protein T9A14.170 - Arabidopsis |
| | | | | | | | thaliana >gi\|4490341\|emb\|CAB38623.1\| |
| | | | | | | | (AL035656) putative protein [Arabidopsis thaliana] |
| | | | | | | | >gi\|7270872\|emb\|CAB80552.1\| (AL161594) |
| | | | | | | | putative protein [Arabidopsis thaliana] |
| 2700 | LIB3602-081-Q6-K6-A5 | 118236 | BLASTX | 164 | 5.00E−11 | 45 | DIHYDRODIPICOLINATE SYNTHASE 1 |
| | | | | | | | PRECURSOR (DHDPS) >gi\|68224\|pir\|\|WZWTH7 |
| | | | | | | | dihydrodipicolinate synthase (EC 4.2.1.52) |
| | | | | | | | precursor (clone pDA17) - wheat |
| | | | | | | | >gi\|170680\|gb\|AAA34263.1\| (M60598) |
| | | | | | | | dihydrodipicolinate synthase [Triticum aestivum] |
| 2701 | LIB3602-120-Q1-K1-D10 | 7437314 | BLASTX | 283 | 4.00E−25 | 50 | peptidylprolyl isomerase (EC 5.2.1.8) - |
| | | | | | | | Synechocystis sp. (PCC 6803) |
| | | | | | | | >gi\|1652923\|dbj\|BAA17841.1\| (D90909) |
| | | | | | | | peptidylprolyl cis-trans isomerase [Synechocystis sp.] |
| 2702 | LIB3602-107-Q1-K1-C2 | 1174592 | BLASTX | 606 | 5.00E−63 | 94 | TUBULIN ALPHA-1 CHAIN |
| | | | | | | | >gi\|2119270\|pir\|\|S60233 tubulin alpha-1 chain - |
| | | | | | | | garden pea >gi\|525332\|gb\|AAA79910.1\| (U12589) |
| | | | | | | | alpha-tubulin [Pisum sativum] |
| 2703 | LIB3602-025-Q6-K1-A7 | 3046907 | BLASTX | 300 | 4.00E−27 | 90 | (AF019886) beta-tubulin [Onchocerca volvulus] |
| 2704 | LIB3602-086-Q6-K1-B12 | 4567243 | BLASTX | 285 | 4.00E−25 | 37 | (AC007070) putative synaptobrevin [Arabidopsis |
| | | | | | | | thaliana] |
| 2705 | LIB3602-095-Q6-K6-C1 | 7509168 | BLASTX | 159 | 8.00E−11 | 65 | hypothetical protein W09C5.1 - Caenorhabditis |
| | | | | | | | elegans >gi\|3880592\|emb\|CAB04941.1\| (Z82077) |
| | | | | | | | cDNA EST EMBL: D72613 comes from this gene; |
| | | | | | | | cDNA EST EMBL: D75538 comes from this gene; |
| | | | | | | | cDNA EST EMBL: D74123 comes from this gene; |
| | | | | | | | cDNA EST yk289c10.5 comes from this |
| 2706 | LIB3602-115-Q1-K1-E4 | 3288823 | BLASTX | 236 | 2.00E−19 | 37 | (AF063852) FUS5 [Arabidopsis thaliana] |
| 2707 | LIB3602-005-Q1-K1-D1 | 3288821 | BLASTX | 168 | 3.00E−12 | 76 | (AF063901) alanine: glyoxylate aminotransferase; |
| | | | | | | | transaminase [Arabidopsis thaliana] |
| | | | | | | | >gi\|4733989\|gb\|AAD28669.1\|AC007209_5 |
| | | | | | | | (AC007209) alanine-glyoxylate aminotransferase |
| | | | | | | | [Arabidopsis thaliana] |
| 2708 | LIB3602-054-Q6-K1-C2 | 6382535 | BLASTX | 196 | 6.00E−15 | 54 | (AC011020) putative protein kinase [Arabidopsis |
| | | | | | | | thaliana] |
| 2709 | LIB3602-005-Q1-K1-H1 | 4506221 | BLASTX | 242 | 3.00E−21 | 45 | proteasome (prosome, macropain) 26S subunit, non- |
| | | | | | | | ATPase, 12 >gi\|7451897\|pir\|\|JC6523 26s proteasom |
| | | | | | | | p55 protein - human >gi\|1945611\|dbj\|BAA19749.1\| |
| | | | | | | | (AB003103) 26S proteasome subunit p55 [Homo |
| | | | | | | | sapiens] |
| 2710 | LIB3602-025-Q6-K1-C9 | 6831664 | BLASTX | 697 | 1.00E−73 | 87 | 40S RIBOSOMAL PROTEIN S5 |
| | | | | | | | >gi\|7440226\|pir\|\|T16965 ribosomal protein S5 - |
| | | | | | | | curled-leaved tobacco |
| | | | | | | | >gi\|1620982\|emb\|CAA70084.1\| (Y08860) 40S |
| | | | | | | | ribosomal protein S5 [Nicotiana plumbaginifolia] |
| 2711 | LIB3602-020-Q6-K1-H7 | 6729038 | BLASTX | 314 | 8.00E−29 | 44 | (AC009177) unknown protein [Arabidopsis |
| | | | | | | | thaliana] |
| 2712 | LIB3602-077-Q6-K6-E6 | 6006879 | BLASTX | 306 | 1.00E−27 | 60 | (AC008153) putative eukaryotic translation |
| | | | | | | | initiation factor 3 subunit [Arabidopsis thaliana] |
| 2713 | LIB3602-029-Q6-K1-G10 | 7543738 | BLASTX | 241 | 4.00E−20 | 46 | (AC020580) hypothetical protein [Arabidopsis |
| | | | | | | | thaliana] |
| 2714 | LIB3602-101-Q1-K1-G4 | 115791 | BLASTX | 210 | 1.00E−16 | 72 | CHLOROPHYLL A-B BINDING PROTEIN 2 |
| | | | | | | | PRECURSOR (LHCII TYPE I CAB-2) (LHCP) |
| | | | | | | | >gi\|81770\|pir\|\|S01961 chlorophyll a/b-binding |
| | | | | | | | protein 2 precursor - soybean |
| | | | | | | | >gi\|18548\|emb\|CAA31418.1\| (X12980) chlorophyll |
| | | | | | | | a/b binding preprotein (AA-33 to 223) [Glycine |
| | | | | | | | max] |
| 2715 | LIB3602-039-Q6-K1-D2 | 6598844 | BLASTX | 327 | 4.00E−30 | 44 | (AC010795) hypothetical protein [Arabidopsis |
| | | | | | | | thaliana] |
| 2716 | LIB3602-116-Q1-K1-F1 | 115796 | BLASTX | 183 | 5.00E−14 | 79 | CHLOROPHYLL A-B BINDING PROTEIN |
| | | | | | | | PRECURSOR (LHCII TYPE I CAB) (LHCP) |
| | | | | | | | >gi\|218174\|dbj\|BAA00537.1\| (D00642) type II |
| | | | | | | | light-harvesting chlorophyll a/b-binding protein |
| | | | | | | | [Oryza sativa] |
| 2717 | LIB3602-095-Q6-K6-D3 | 4759036 | BLASTX | 167 | 3.00E−13 | 35 | regucalcin (senescence marker protein-30) |
| | | | | | | | >gi\|3334328\|sp\|Q15493\|SM30_HUMAN |
| | | | | | | | SENESCENCE MARKER PROTEIN-30 (SMP-30) |
| | | | | | | | (REGUCALCIN) (RC) >gi\|2136176\|pir\|\|I52491 |
| | | | | | | | SMP-30 - human >gi\|7429379\|pir\|\|S60035 |
| | | | | | | | senescence marker protein 30 - human |
| | | | | | | | >gi\|1072312\|dbj\|BAA06602 |
| 2718 | LIB3602-046-Q6-K1-B1 | 7488941 | BLASTX | 668 | 3.00E−70 | 82 | adenosylhomocysteinase (EC 3.3.1.1) - parsley |
| | | | | | | | >gi\|169661\|gb\|AAA33855.1\| (M62756) S- |
| | | | | | | | adenosylhomocysteine hydrolase [Petroselinum |
| | | | | | | | crispum] |
| 2719 | LIB3602-045-Q6-K1-G7 | 6598834 | BLASTX | 618 | 2.00E−64 | 78 | (AC010795) putative replication factor C |
| | | | | | | | [Arabidopsis thaliana] |
| 2720 | LIB3602-049-Q6-K1-F12 | 3023500 | BLASTX | 177 | 1.00E−12 | 63 | ATP-DEPENDENT CLP PROTEASE |
| | | | | | | | PROTEOLYTIC SUBUNIT 2 (ENDOPEPTIDASE |
| | | | | | | | CLP 2) >gi\|2351823\|gb\|AAB68677.1\| (U92039) |
| | | | | | | | ATP-dependent Clp protease, proteolytic subunit |
| | | | | | | | [Synechococcus PCC7942] |
| 2721 | LIB3602-014-Q6-K1-C12 | 6598677 | BLASTX | 192 | 2.00E−14 | 36 | (AC007135) hypothetical protein [Arabidopsis |
| | | | | | | | thaliana] |
| 2722 | LIB3602-007-Q1-K1-F7 | 1710008 | BLASTX | 255 | 1.00E−21 | 53 | GTP-BINDING NUCLEAR PROTEIN RAN1B |
| | | | | | | | >gi\|1370205\|emb\|CAA98188.1\| (Z73960) RAN1B |
| | | | | | | | [Lotus japonicus] |
| 2723 | LIB3602-111-Q1-K1-D12 | 2501056 | BLASTX | 323 | 1.00E−29 | 48 | SERYL-TRNA SYNTHETASE (SERINE--TRNA |
| | | | | | | | LIGASE) (SERRS) >gi\|2129737\|pir\|\|S71293 serine- |
| | | | | | | | tRNA ligase (EC 6.1.1.11) - Arabidopsis thaliana |
| | | | | | | | >gi\|1359497\|emb\|CAA94388.1\| (Z70313) seryl- |
| | | | | | | | tRNA Synthetase [Arabidopsis thaliana] |
| 2724 | LIB3602-109-Q1-K1-E8 | 7491321 | BLASTX | 164 | 5.00E−11 | 32 | hypothetical protein SPBC119.09c - fission yeast |
| | | | | | | | (Schizosaccharomyces pombe) |
| | | | | | | | >gi\|2959370\|emb\|CAA17924.1\| (AL022117) |
| | | | | | | | hypothetical protein. [Schizosaccharomyces pombe] |
| 2725 | LIB3602-014-Q6-K1-E3 | 2501062 | BLASTX | 147 | 3.00E−09 | 43 | THREONYL-TRNA SYNTHETASE |
| | | | | | | | (THREONINE--TRNA LIGASE) (THRRS) |
| | | | | | | | >gi\|7437520\|pir\|\|S76615 hypothetical protein - |
| | | | | | | | Synechocystis sp. (strain PCC 6803) |
| | | | | | | | >gi\|1001722\|dbj\|BAA10559.1\| (D64004) threonyl- |
| | | | | | | | tRNA synthetase [Synechocystis sp.] |
| 2726 | LIB3602-117-Q1-K1-H3 | 4210330 | BLASTX | 354 | 2.00E−33 | 55 | (AJ223802) 2-oxoglutarate dehydrogenase, E1 |
| | | | | | | | subunit [Arabidopsis thaliana] |
| 2727 | LIB3602-018-Q6-K1-F1 | 4098989 | BLASTX | 194 | 2.00E−14 | 36 | (U81498) phenylalkylamine binding protein |
| | | | | | | | homolog [Arabidopsis thaliana] |
| 2728 | LIB3602-061-Q6-K1-H2 | 4210334 | BLASTX | 314 | 9.00E−29 | 68 | (AJ223804) 2-oxoglutarate dehydrogenase, E3 |
| | | | | | | | subunit [Arabidopsis thaliana] |
| 2729 | LIB3602-014-Q6-K1-B1 | 4432812 | BLASTX | 204 | 1.00E−15 | 34 | (AC006593) putative cysteinyl-tRNA synthetase |
| | | | | | | | [Arabidopsis thaliana] |
| 2730 | LIB3602-023-Q6-K1-H7 | 5531416 | BLASTX | 355 | 2.00E−33 | 48 | (AJ243758) translocon Tic40 [Pisum sativum] |
| 2731 | LIB3602-028-Q6-K1-C10 | 4049522 | BLASTX | 414 | 2.00E−40 | 75 | (AJ011516) fructose-bisphosphate aldolase |
| | | | | | | | [Scherffelia dubia] |
| 2732 | LIB3602-073-Q1-K1-D5 | 2564066 | BLASTX | 143 | 8.00E−09 | 65 | (D45900) LEDI-3 protein [Lithospermum |
| | | | | | | | erythrorhizon] |
| 2733 | LIB3602-081-Q6-K6-C9 | 2497858 | BLASTX | 209 | 3.00E−16 | 45 | MALATE DEHYDROGENASE, |
| | | | | | | | MITOCHONDRIAL PRECURSOR |
| | | | | | | | >gi\|7431169\|pir\|\|T08077 malate dehydrogenase (EC |
| | | | | | | | 1.1.1.37) precursor, mitochondrial - |
| | | | | | | | Chlamydomonas reinhardtii |
| | | | | | | | >gi\|1145722\|gb\|AAA84971.1\| (U40212) malate |
| | | | | | | | dehydrogenase [Chlamydomonas reinhardtii] |
| 2734 | LIB3602-100-Q6-K1-B11 | 6056206 | BLASTX | 211 | 6.00E−20 | 53 | (AC009400) putative kinesin-like centromere |
| | | | | | | | protein [Arabidopsis thaliana] |
| 2735 | LIB3602-118-Q1-K1-H10 | 2149901 | BLASTX | 186 | 1.00E−13 | 51 | (U94707) unknown [Enterococcus faecalis] |
| 2736 | LIB3602-018-Q6-K1-D2 | 6729045 | BLASTX | 163 | 6.00E−11 | 43 | (AC009177) putative aminopeptidase [Arabidopsis |
| | | | | | | | thaliana] |
| 2737 | LIB3602-062-Q6-K1-C9 | 7447193 | BLASTX | 357 | 1.00E−33 | 43 | conserved hypothetical protein aq_1660 - Aquifex |
| | | | | | | | aeolicus >gi\|2983997\|gb\|AAC07543.1\| (AE000749) |
| | | | | | | | hypothetical protein [Aquifex aeolicus] |
| 2738 | LIB3602-111-Q1-K1-B6 | 132894 | BLASTX | 182 | 3.00E−13 | 58 | CYANELLE 50S RIBOSOMAL PROTEIN L33 |
| | | | | | | | >gi\|71357\|pir\|\|R5KT33 ribosomal protein L33 - |
| | | | | | | | Cyanophora paradoxa cyanelle |
| | | | | | | | >gi\|11401\|emb\|CAA35534.1\| (X17498) L33 |
| | | | | | | | ribosomal protein (AA 1-64) [Cyanophora |
| | | | | | | | paradoxa] >gi\|1016146\|gb\|AAA81233.1\| (U30821) |
| | | | | | | | ribosomal protein L3 |
| 2739 | LIB3602-036-Q6-K1-H9 | 6978417 | BLASTX | 162 | 1.00E−10 | 30 | (AC005941) L5204.2 [Leishmania major] |
| 2740 | LIB3602-010-Q6-K1-A11 | 963025 | BLASTN | 34 | 3.00E−09 | 93 | D. hydei gene for histone H3.3B |
| 2741 | LIB3602-057-Q6-K1-E6 | 3929364 | BLASTX | 551 | 3.00E−56 | 84 | NADH-UBIQUINONE OXIDOREDUCTASE 23 KD |
| | | | | | | | SUBUNIT PRECURSOR (COMPLEX I- |
| | | | | | | | 23 KD) (CI-23 KD) (COMPLEX I-28.5 KD) (CI- |
| | | | | | | | 28.5 KD) >gi\|1076356\|pir\|\|S52380 NADH |
| | | | | | | | dehydrogenase (ubiquinone) (EC 1.6.5.3) chain |
| | | | | | | | TYKY precursor - Arabidopsis thaliana |
| | | | | | | | >gi\|666977\|emb\|CAA59061.1\| (X |
| 2742 | LIB3602-086-Q6-K1-B6 | 226263 | BLASTX | 154 | 8.00E−10 | 35 | chlorophyll a/b binding protein [Glycine max] |
| 2743 | LIB3602-040-Q6-K1-C5 | 2500139 | BLASTX | 162 | 2.00E−11 | 56 | PEPTIDE CHAIN RELEASE FACTOR 1 (RF-1) |
| | | | | | | | >gi\|7428981\|pir\|\|S76914 translation releasing factor |
| | | | | | | | RF-1 - Synechocystis sp. (strain PCC 6803) |
| | | | | | | | >gi\|1653916\|dbj\|BAA18826.1\| (D90917) peptide |
| | | | | | | | chain release factor [Synechocystis sp.] |
| 2744 | LIB3602-042-Q6-K1-G3 | 3283057 | BLASTX | 163 | 5.00E−11 | 50 | (AF054617) one helix protein [Arabidopsis |
| | | | | | | | thaliana] >gi\|7340686\|emb\|CAB82985.1\| |
| | | | | | | | (AL162508) one helix protein (OHP) [Arabidopsis |
| | | | | | | | thaliana] |
| 2745 | LIB3602-016-Q6-K1-H7 | 5107826 | BLASTX | 219 | 1.00E−17 | 38 | (AF149413) similar to malate dehydrogenases; |
| | | | | | | | Pfam PF00390, Score = 1290.5. E = 0, N = 1 |
| | | | | | | | [Arabidopsis thaliana] |
| 2746 | LIB3602-114-Q1-K1-B6 | 6831634 | BLASTX | 206 | 4.00E−16 | 59 | 50S RIBOSOMAL PROTEIN L6 |
| | | | | | | | >gi\|7440700\|pir\|\|C72054 L6 ribosomal protein - |
| | | | | | | | Chlamydia pneumoniae (strain CWL029) |
| | | | | | | | >gi\|4376931\|gb\|AAD18772.1\| (AE001647) L6 |
| | | | | | | | Ribosomal Protein [Chlamydophila pneumoniae] |
| | | | | | | | >gi\|7189048\|gb\|AAF37997.1\| (AE002173) |
| | | | | | | | ribosomal protein L6 [ |
| 2747 | LIB3602-067-Q1-K1-B10 | 6715645 | BLASTX | 248 | 5.00E−21 | 66 | (AC007323) T25K16.8 [Arabidopsis thaliana] |
| 2748 | LIB3602-051-Q6-K1-H12 | 7445539 | BLASTX | 276 | 2.00E−24 | 48 | aldose 1-epimerase - Thermotoga maritima (strain |
| | | | | | | | MSB8) >gi\|4980780\|gb\|AAD35370.1\|AE001710_5 |
| | | | | | | | (AE001710) aldose 1-epimerase [Thermotoga |
| | | | | | | | maritima] |
| 2749 | LIB3602-057-Q6-K1-A12 | 4325370 | BLASTX | 673 | 1.00E−70 | 63 | (AF128396) similar to human phosphotyrosyl |
| | | | | | | | phosphatase activator PTPA (GB: X73478) |
| | | | | | | | [Arabidopsis thaliana] |
| | | | | | | | >gi\|7267538\|emb\|CAB78020.1\| (AL161513) |
| | | | | | | | putative phosphotyrosyl phosphatase activator |
| | | | | | | | protein [Arabidopsis thaliana] |
| 2750 | LIB3602-017-Q6-K1-A11 | 5869803 | BLASTX | 257 | 6.00E−22 | 31 | (X64418) kurz protein [Drosophila melanogaster] |
| 2751 | LIB3602-085-Q6-K1-B5 | 7301290 | BLASTX | 199 | 4.00E−15 | 42 | (AE003751) CG11851 gene product [Drosophila |
| | | | | | | | melanogaster] |
| 2752 | LIB3602-051-Q6-K1-B11 | 7447302 | BLASTX | 165 | 3.00E−11 | 38 | probable glutathione transferase (EC 2.5.1.18), 2,4- |
| | | | | | | | D inducible - soybean |
| | | | | | | | >gi\|2920666\|gb\|AAC18566.1\| (AF048978) 2,4-D |
| | | | | | | | inducible glutathione S-transferase [Glycine max] |
| 2753 | LIB3602-109-Q1-K1-H7 | 1354764 | BLASTX | 145 | 4.00E−09 | 58 | (U56935) beta-1,3-glucanase IIa [Oerskovia |
| | | | | | | | xanthineolytica] |
| 2754 | LIB3602-043-Q6-K1-F5 | 5734642 | BLASTX | 153 | 6.00E−10 | 33 | (AP000391) ESTs C22657(S0014), C22656(S0014) |
| | | | | | | | correspond to a region of the predicted gene.; |
| | | | | | | | Similar to receptor protein kinase, ERECTA |
| | | | | | | | (AC004484) [Oryza sativa] |
| | | | | | | | >gi\|6006357\|dbj\|BAA84787.1\| (AP000559) ESTs |
| | | | | | | | C22657(S0014), C22656(S0014) correspond to a |
| | | | | | | | region |
| 2755 | LIB3602-012-Q6-K1-G7 | 3763918 | BLASTX | 573 | 6.00E−59 | 66 | (AC004450) 3-isopropylmalate dehydratase, small |
| | | | | | | | subunit [Arabidopsis thaliana] |
| 2756 | LIB3602-040-Q6-K1-F6 | 6642738 | BLASTX | 191 | 3.00E−14 | 38 | (AF113007) PRO0066 [Homo sapiens] |
| 2757 | LIB3602-064-Q1-K6-D6 | 4567273 | BLASTX | 383 | 1.00E−36 | 50 | (AC006841) putative vacuolar proton-ATPase |
| | | | | | | | subunit 1 [Arabidopsis thaliana] |
| 2758 | LIB3602-117-Q1-K1-H10 | 3757515 | BLASTX | 204 | 8.00E−16 | 64 | (AC005167) hypothetical protein [Arabidopsis |
| | | | | | | | thaliana] |
| 2759 | LIB3602-034-Q6-K1-B8 | 7385113 | BLASTX | 196 | 8.00E−15 | 43 | (AF222766) ankyrin 1 [Bos taurus] |
| 2760 | LIB3602-047-Q6-K1-A4 | 7487883 | BLASTX | 218 | 2.00E−17 | 36 | hypothetical protein T9J22.21 - Arabidopsis |
| | | | | | | | thaliana >gi\|2739379\|gb\|AAC14502.1\| (AC002505) |
| | | | | | | | hypothetical protein [Arabidopsis thaliana] |
| 2761 | LIB3602-071-Q1-K1-C11 | 2500318 | BLASTX | 258 | 6.00E−22 | 63 | 50S RIBOSOMAL PROTEIN L28 |
| | | | | | | | >gi\|7441051\|pir\|\|S74715 ribosomal protein L28 - |
| | | | | | | | Synechocystis sp. (strain PCC 6803) |
| | | | | | | | >gi\|1651940\|dbj\|BAA16866.1\| (D90901) 50S |
| | | | | | | | ribosomal protein L28 [Synechocystis sp.] |
| 2762 | LIB3602-068-Q1-K1-A10 | 7303420 | BLASTX | 435 | 1.00E−42 | 48 | (AE003821) CG8778 gene product [Drosophila |
| | | | | | | | melanogaster] |
| 2763 | LIB3602-041-Q6-K1-B6 | 7510133 | BLASTX | 231 | 7.00E−19 | 59 | hypothetical protein Y52B11A.3 - Caenorhabditis |
| | | | | | | | elegans >gi\|3881161\|emb\|CAA21721.1\| |
| | | | | | | | (AL032654) similar to Heme-binding domain in |
| | | | | | | | cytochrome b5 and oxidoreductases [Caenorhabditis |
| | | | | | | | elegans] |
| 2764 | LIB3602-078-Q6-K6-E9 | 5281018 | BLASTX | 245 | 3.00E−21 | 63 | (Z97339) OBP33pep like protein [Arabidopsis |
| | | | | | | | thaliana] >gi\|7268299\|emb\|CAB78594.1\| |
| | | | | | | | (AL161541) OBP33pep like protein [Arabidopsis |
| | | | | | | | thaliana] |
| 2765 | LIB3602-077-Q6-K6-A10 | 1709243 | BLASTX | 254 | 7.00E−22 | 70 | NUCLEOSIDE DIPHOSPHATE KINASE NBR-B |
| | | | | | | | (NDK NBR-B) (NDP KINASE NBR-B) |
| | | | | | | | >gi\|4389319\|pdb\|1BE4\|C Chain C, Nucleoside |
| | | | | | | | Diphosphate Kinase Isoform B From Bovine Retina |
| | | | | | | | >gi\|1064897\|emb\|CAA63533.1\| (X92957) |
| | | | | | | | nucleoside-diphosphate kinase NBR-B [Bos taurus] |
| 2766 | LIB3602-032-Q6-K1-H11 | 3970652 | BLASTX | 148 | 2.00E−09 | 48 | (X77499) amino acid permease [Arabidopsis |
| | | | | | | | thaliana] |
| 2767 | LIB3602-072-Q1-K1-E12 | 7489826 | BLASTX | 457 | 2.00E−45 | 51 | starch synthase DULL1 - maize |
| | | | | | | | >gi\|3057120\|gb\|AAC14014.1\| (AF023159) starch |
| | | | | | | | synthase DULL1 [Zea mays] |
| 2768 | LIB3602-066-Q1-K6-F5 | 7295126 | BLASTX | 436 | 7.00E−43 | 54 | (AE003556) CG7197 gene product [Drosophila |
| | | | | | | | melanogaster] |
| 2769 | LIB3602-025-Q6-K1-D1 | 6729017 | BLASTX | 187 | 9.00E−14 | 48 | (AC009177) unknown protein [Arabidopsis |
| | | | | | | | thaliana] |
| 2770 | LIB3602-017-Q6-K1-C4 | 4774163 | BLASTX | 305 | 1.00E−27 | 36 | (AB026909) F1-ATP synthase delta subunit |
| | | | | | | | [Ipomoea batatas] |
| 2771 | LIB3602-100-Q1-K1-D2 | 2498712 | BLASTX | 344 | 4.00E−32 | 47 | ORIGIN RECOGNITION COMPLEX SUBUNIT 2 |
| | | | | | | | (XORC2) >gi\|7512175\|pir\|\|S68447 origin |
| | | | | | | | recognition complex chain orc2 - African clawed |
| | | | | | | | frog >gi\|1177822\|gb\|AAA96391.1\| (U31984) |
| | | | | | | | XORC2 [Xenopus laevis] |
| | | | | | | | >gi\|1586824\|prf\|\|2204391A orc-2-related protein |
| | | | | | | | [Xenopus laevis] |
| 2772 | LIB3602-069-Q1-K1-E1 | 125577 | BLASTX | 318 | 5.00E−29 | 87 | PHOSPHORIBULOKINASE PRECURSOR |
| | | | | | | | (PHOSPHOPENTOKINASE) (PRKASE) (PRK) |
| | | | | | | | >gi\|7434254\|pir\|\|T08167 phosphoribulokinase (EC |
| | | | | | | | 2.7.1.19) precursor - Chlamydomonas reinhardtii |
| | | | | | | | >gi\|167432\|gb\|AAA33090.1\| (M36123) |
| | | | | | | | phosphoribulokinase [Chlamydomonas reinhardtii] |
| | | | | | | | >gi\|710740 |
| 2773 | LIB3602-076-Q6-K6-B1 | 6002102 | BLASTX | 172 | 6.00E−12 | 45 | (AJ249833) Acyl-CoA binding protein (ACBP) |
| | | | | | | | [Digitalis lanata] |
| 2774 | LIB3602-049-Q6-K1-C9 | 3676298 | BLASTX | 218 | 2.00E−17 | 35 | (U97104) membrane transporter [Bactrocera tryoni] |
| 2775 | LIB3602-059-Q6-K1-G6 | 6688812 | BLASTX | 684 | 5.00E−72 | 71 | (AJ248327) L3 Ribosomal protein [Medicago sativa |
| | | | | | | | subsp. x varia] |
| 2776 | LIB3602-119-Q1-K1-E3 | 7447939 | BLASTX | 171 | 9.00E−16 | 53 | hypothetical protein - Synechocystis sp. (strain PCC |
| | | | | | | | 6803) >gi\|1001355\|dbj\|BAA10842.1\| (D64006) |
| | | | | | | | auxin-induced protein [Synechocystis sp.] |
| 2777 | LIB3602-007-Q1-K1-H9 | 320553 | BLASTX | 402 | 8.00E−39 | 62 | anthranilate synthase (EC 4.1.3.27) alpha-2 chain - |
| | | | | | | | Arabidopsis thaliana |
| 2778 | LIB3602-015-Q6-K1-H4 | 2598039 | BLASTX | 255 | 1.00E−21 | 37 | (AJ001273) manganese resistance 1 protein |
| | | | | | | | [Saccharomyces cerevisiae] |
| 2779 | LIB3602-078-Q6-K6-B3 | 124374 | BLASTX | 237 | 2.00E−19 | 34 | ACETOLACTATE SYNTHASE |
| | | | | | | | (ACETOHYDROXY-ACID SYNTHASE) (ALS) |
| | | | | | | | >gi\|96560\|pir\|\|JC1218 acetolactate synthase (EC |
| | | | | | | | 4.1.3.18), FAD-independent - Klebsiella |
| | | | | | | | pneumoniae >gi\|149211\|gb\|AAA25079.1\| (M73842) |
| | | | | | | | acetolactate synthase [Klebsiella pneumoniae] |
| 2780 | LIB3602-071-Q1-K1-G7 | 7487731 | BLASTX | 277 | 3.00E−24 | 47 | hypothetical protein T5L19.160 - Arabidopsis |
| | | | | | | | thaliana >gi\|4539006\|emb\|CAB39627.1\| |
| | | | | | | | (AL049481) putative protein [Arabidopsis thaliana] |
| | | | | | | | >gi\|7267699\|emb\|CAB78126.1\| (AL161516) |
| | | | | | | | putative protein [Arabidopsis thaliana] |
| 2781 | LIB3602-057-Q6-K1-C8 | 7298683 | BLASTX | 160 | 2.00E−14 | 73 | (AE003666) CG2493 gene product [Drosophila |
| | | | | | | | melanogaster] |
| 2782 | LIB3602-023-Q6-K1-D3 | 2130089 | BLASTX | 159 | 2.00E−10 | 55 | 2-oxoglutarate/malate translocator (clone OMT103), |
| | | | | | | | mitochondrial membrane - proso millet |
| | | | | | | | >gi\|1100743\|dbj\|BAA08105.1\| (D45075) 2- |
| | | | | | | | oxoglutarate/malate translocator [Panicum |
| | | | | | | | miliaceum] |
| 2783 | LIB3602-071-Q1-K1-D11 | 6562261 | BLASTX | 154 | 7.00E−10 | 55 | (AL132980) putative protein [Arabidopsis thaliana] |
| 2784 | LIB3602-012-Q6-K1-E7 | 6969002 | BLASTX | 155 | 7.00E−10 | 32 | (AL139079) putative oxidoreductase |
| | | | | | | | [Campylobacter jejuni] |
| 2785 | LIB3602-011-Q6-K1-E10 | 7488455 | BLASTX | 341 | 8.00E−32 | 49 | hydroxymethylpyrimidine kinase (EC 2.7.1.49)/ |
| | | | | | | | thiamin-phosphate pyrophosphorylase (EC 2.5.1.3) - |
| | | | | | | | rape >gi\|2746079\|gb\|AAC31298.1\| (AF015310) |
| | | | | | | | BTH1 [Brassica napus] |
| 2786 | LIB3602-010-Q6-K1-D7 | 7484895 | BLASTX | 172 | 6.00E−12 | 49 | cytochrome P450 homolog F13P17.33 - |
| | | | | | | | Arabidopsis thaliana >gi\|3128210\|gb\|AAC26690.1\| |
| | | | | | | | (AC004077) putative cytochrome P450 |
| | | | | | | | [Arabidopsis thaliana] |
| 2787 | LIB3602-009-Q6-K1-C8 | 6016183 | BLASTX | 478 | 7.00E−48 | 55 | PHOTOSYSTEM II STABILITY/ASSEMBLY |
| | | | | | | | FACTOR HCF136 >gi\|3559807\|emb\|CAA75723.1\| |
| | | | | | | | (Y15628) HCF136 protein [Arabidopsis thaliana] |
| 2788 | LIB3602-114-Q1-K1-B5 | 2190717 | BLASTN | 38 | 1.00E−11 | 82 | Cloning vector pBI-GL, complete sequence |
| 2789 | LIB3602-067-Q1-K1-C10 | 5107528 | BLASTX | 167 | 2.00E−11 | 36 | Probing The Substrate Specificity Of The |
| | | | | | | | Intracellular Brain Platelet-Activating Factor |
| | | | | | | | Acetylhydrolase |
| 2790 | LIB3602-041-Q6-K1-D8 | 6572215 | BLASTX | 247 | 1.00E−20 | 44 | (Z83844) dJ37E16.5 (novel protein similar to |
| | | | | | | | nitrophenylphosphatases from various organisms) |
| | | | | | | | [Homo sapiens] |
| 2791 | LIB3602-050-Q6-K1-C6 | 2829532 | BLASTX | 268 | 3.00E−23 | 43 | HYPOTHETICAL 48.5 KD PROTEIN RV1407 |
| | | | | | | | >gi\|7445108\|pir\|\|D70901 probable fmu protein - |
| | | | | | | | Mycobacterium tuberculosis (strain H37RV) |
| | | | | | | | >gi\|1542915\|emb\|CAB02186.1\| (Z80108) fmu |
| | | | | | | | [Mycobacterium tuberculosis] |
| 2792 | LIB3602-090-Q6-K6-C4 | 127210 | BLASTX | 353 | 3.00E−33 | 69 | METHYLMALONATE-SEMIALDEHYDE |
| | | | | | | | DEHYDROGENASE [ACYLATING] (MMSDH) |
| | | | | | | | >gi\|281523\|pir\|\|B42902 methylmalonate- |
| | | | | | | | semialdehyde dehydrogenase (acylating) (EC |
| | | | | | | | 1.2.1.27) - Pseudomonas aeruginosa (ATCC 15692) |
| | | | | | | | >gi\|151362\|gb\|AAA25891.1\| (M84911) |
| | | | | | | | methylmalonate semialdehyde d |
| 2793 | LIB3602-102-Q1-K1-C5 | 7433831 | BLASTX | 187 | 4.00E−28 | 63 | sucrose synthase (EC 2.4.1.13) T2H3.8 - |
| | | | | | | | Arabidopsis thaliana >gi\|3377802\|gb\|AAC28175.1\| |
| | | | | | | | (AF075597) Similar to sucrose synthase; T2H3.8 |
| | | | | | | | [Arabidopsis thaliana] |
| 2794 | LIB3602-027-Q6-K1-F2 | 6572072 | BLASTX | 154 | 7.00E−10 | 49 | (AL133452) putative protein [Arabidopsis thaliana] |
| 2795 | LIB3602-107-Q1-K1-F6 | 2129826 | BLASTX | 194 | 2.00E−14 | 30 | phragmoplastin 5 - soybean |
| | | | | | | | >gi\|1218004\|gb\|AAC49183.1\| (U36430) SDL5A |
| | | | | | | | [Glycine max] |
| 2796 | LIB3602-100-Q1-K1-D8 | 1708059 | BLASTX | 309 | 8.00E−35 | 51 | GLUTATHIONE REDUCTASE, CHLOROPLAST |
| | | | | | | | PRECURSOR (GR) (GRASE) |
| | | | | | | | >gi\|481593\|pir\|\|S38908 glutathione reductase |
| | | | | | | | (NADPH) (EC 1.6.4.2) - common tobacco |
| | | | | | | | (fragment) >gi\|431955\|emb\|CAA53925.1\| (X76293) |
| | | | | | | | glutathione reductase (NADPH) [Nicotiana |
| | | | | | | | tabacum] |
| 2797 | LIB3602-006-Q1-K1-E11 | 7291436 | BLASTX | 151 | 1.00E−23 | 90 | (AE003458) CG4046 gene product [Drosophila |
| | | | | | | | melanogaster] |
| 2798 | LIB3602-011-Q6-K1-E11 | 4028549 | BLASTX | 170 | 1.00E−11 | 27 | (AF093690) ubiquitin hydrolase B [Dictyostelium |
| | | | | | | | discoideum] |
| 2799 | LIB3602-037-Q6-K1-G10 | 7434563 | BLASTX | 407 | 1.00E−39 | 56 | aspartate kinase (EC 2.7.2.4)/homoserine |
| | | | | | | | dehydrogenase (EC 1.1.1.3) precursor - soybean |
| | | | | | | | (fragment) >gi\|2970556\|gb\|AAC05983.1\| |
| | | | | | | | (AF049708) aspartokinase-homoserine |
| | | | | | | | dehydrogenase [Glycine max] |
| 2800 | LIB3602-071-Q1-K1-E6 | 7021737 | BLASTX | 166 | 3.00E−11 | 60 | (AC024081) hypothetical protein [Arabidopsis |
| | | | | | | | thaliana] |
| 2801 | LIB3602-077-Q6-K6-H11 | 4204793 | BLASTX | 474 | 2.00E−47 | 48 | (U52079) P-glycoprotein [Solanum tuberosum] |
| 2802 | LIB3602-081-Q6-K6-A7 | 6006398 | BLASTN | 41 | 1.00E−13 | 86 | Aspergillus niger mRNA for ribosomal protein S28 |
| | | | | | | | (rps28 gene) |
| 2803 | LIB3602-088-Q6-K6-D9 | 7508349 | BLASTX | 186 | 2.00E−20 | 54 | hypothetical protein T23H2.6 - Caenorhabditis |
| | | | | | | | elegans >gi\|2731374\|gb\|AAC48203.1\| (U80033) |
| | | | | | | | T23H2.6 gene product [Caenorhabditis elegans] |
| 2804 | LIB3602-010-Q6-K1-D12 | 6319917 | BLASTX | 151 | 7.00E−10 | 36 | protein kinase; Ssk22p |
| | | | | | | | >gi\|140530\|sp\|P25390\|SS22_YEAST |
| | | | | | | | SERINE/THREONINE PROTEIN KINASE SSK22 |
| | | | | | | | >gi\|83250\|pir\|\|S19488 probable membrane protein |
| | | | | | | | YCR073c - yeast (Saccharomyces cerevisiae) |
| | | | | | | | >gi\|1907212\|emb\|CAA42271.1\| (X59720) |
| | | | | | | | YCR073c, len: 1314 [Saccharomyces c |
| 2805 | LIB3602-120-Q1-K1-A2 | 1076820 | BLASTX | 149 | 1.00E−09 | 96 | phosphoglycerate mutase (EC 5.4.2.1) - maize |
| 2806 | LIB3602-087-Q6-K1-A5 | 7387793 | BLASTX | 206 | 6.00E−16 | 34 | TRANSLATION INITIATION FACTOR IF-3 |
| 2807 | LIB3602-043-Q6-K1-A10 | 99898 | BLASTX | 210 | 1.00E−16 | 48 | DNA-directed RNA polymerase (EC 2.7.7.6) largest |
| | | | | | | | chain (isoform B1) - soybean (fragment) |
| 2808 | LIB3602-049-Q6-K1-F2 | 4733939 | BLASTX | 492 | 1.00E−49 | 71 | (AF068686) geranylgeranyl hydrogenase [Glycine |
| | | | | | | | max] |
| 2809 | LIB3602-053-Q6-K1-B2 | 337930 | BLASTX | 402 | 4.00E−39 | 67 | (M22146) scar protein [Homo sapiens] |
| 2810 | LIB3602-001-P1-K6-D8 | 7446109 | BLASTX | 265 | 9.00E−23 | 48 | RNA helicase RH16 - Arabidopsis thaliana |
| | | | | | | | >gi\|5123708\|emb\|CAB45452.1\| (AL079347) RNA |
| | | | | | | | helicase (RH16) [Arabidopsis thaliana] |
| | | | | | | | >gi\|7270442\|emb\|CAB80208.1\| (AL161586) RNA |
| | | | | | | | helicase (RH16) [Arabidopsis thaliana] |
| 2811 | LIB3602-091-Q6-K6-F3 | 1262849 | BLASTX | 268 | 3.00E−23 | 76 | (U51633) type 1 light-harvesting chlorophyll a/b- |
| | | | | | | | binding polypeptide [Pinus palustris] |
| 2812 | LIB3602-037-Q6-K1-B9 | 3914594 | BLASTX | 167 | 2.00E−11 | 65 | RIBULOSE BISPHOSPHATE CARBOXYLASE |
| | | | | | | | SMALL CHAIN 6 PRECURSOR (RUBISCO |
| | | | | | | | SMALL SUBUNIT 6) |
| | | | | | | | >gi\|2654375\|emb\|CAA82266.1\| (Z28640) |
| | | | | | | | ribulosebiphosphate carboxylase, small subunit |
| | | | | | | | [Acetabularia cliftonii] |
| 2813 | LIB3602-012-Q6-K1-B2 | 7434369 | BLASTX | 194 | 2.00E−14 | 44 | calcium-dependent protein kinase (EC 2.7.1.—) - |
| | | | | | | | sweet potato >gi\|1552214\|dbj\|BAA13440.1\| |
| | | | | | | | (D87707) calcium dependent protein kinase |
| | | | | | | | [Ipomoea batatas] |
| 2814 | LIB3602-013-Q6-K1-B11 | 7520797 | BLASTX | 228 | 1.00E−18 | 43 | phosphate permease PAB0927 - Pyrococcus abyssi |
| | | | | | | | (strain Orsay) >gi\|5458819\|emb\|CAB50306.1\| |
| | | | | | | | (AJ248287) phosphate permease [Pyrococcus |
| | | | | | | | abyssi] |
| 2815 | LIB3602-001-P1-K6-D7 | 7522579 | BLASTX | 188 | 5.00E−14 | 36 | similar to S. cerevisiae sur1 protein - fission yeast |
| | | | | | | | (Schizosaccharomyces pombe) (fragment) |
| | | | | | | | >gi\|2408057\|emb\|CAB16259.1\| (Z99165) similar to |
| | | | | | | | S. cerevisiae sur1 protein [Schizosaccharomyces |
| | | | | | | | pombe] |
| 2816 | LIB3602-111-Q1-K1-E5 | 6630689 | BLASTX | 453 | 5.00E−54 | 63 | (AP000969) ESTs D39011(R0609), |
| | | | | | | | AU032023(R3215) correspond to a region of the |
| | | | | | | | predicted gene.; Similar to 26S proteasome, non- |
| | | | | | | | ATPase subunit. (Y13071) [Oryza sativa] |
| 2817 | LIB3602-070-Q1-K1-G5 | 6912238 | BLASTX | 334 | 7.00E−31 | 46 | antioxidant enzyme B166 |
| | | | | | | | >gi\|6103724\|gb\|AAF03750.1\|AF110731_1 |
| | | | | | | | (AF110731) antioxidant enzyme B166 [Homo |
| | | | | | | | sapiens] |
| 2818 | LIB3602-078-Q6-K6-D5 | 7489167 | BLASTX | 157 | 2.00E−10 | 94 | nascent polypeptide associated complex alpha chain - |
| | | | | | | | common tobacco (fragment) |
| | | | | | | | >gi\|1658271\|gb\|AAB18266.1\| (U74622) nascent |
| | | | | | | | polypeptide associated complex alpha chain |
| | | | | | | | [Nicotiana tabacum] |
| 2819 | LIB3602-077-Q6-K6-D10 | 628056 | BLASTX | 245 | 1.00E−20 | 70 | FK506-binding protein - Botryllus schlosseri |
| | | | | | | | >gi\|435471\|emb\|CAA53594.1\| (X76006) FK506- |
| | | | | | | | binding protein [Botryllus schlosseri] |
| 2820 | LIB3602-066-Q1-K6-F2 | 6679088 | BLASTX | 505 | 6.00E−51 | 72 | nicotinamide nucleotide transhydrogenase |
| | | | | | | | >gi\|6225774\|sp\|Q61941\|NNTM_MOUSE NAD(P) |
| | | | | | | | TRANSHYDROGENASE, MITOCHONDRIAL |
| | | | | | | | PRECURSOR (PYRIDINE NUCLEOTIDE |
| | | | | | | | TRANSHYDROGENASE) (NICOTINAMIDE |
| | | | | | | | NUCLEOTIDE TRANSHYDROGENASE) |
| | | | | | | | >gi\|1083428\|pir\|\|S54876 NAD(P)+ |
| | | | | | | | transhydrogenase ( |
| 2821 | LIB3602-028-Q6-K1-H1 | 4325041 | BLASTX | 219 | 2.00E−17 | 66 | (AF117339) FtsH-like protein Pftf precursor |
| | | | | | | | [Nicotiana tabacum] |
| 2822 | LIB3602-120-Q1-K1-H2 | 6273391 | BLASTX | 221 | 8.00E−18 | 59 | (AF196333) nonhistone protein 6 [Candida |
| | | | | | | | albicans] |
| 2823 | LIB3602-046-Q6-K1-E7 | 1175367 | BLASTX | 255 | 9.00E−22 | 63 | HYPOTHETICAL 36.7 KD PROTEIN C2F7.02C |
| | | | | | | | IN CHROMOSOME I >gi\|2130371\|pir\|\|S58146 |
| | | | | | | | hypothetical protein SPAC2F7.02c - fission yeast |
| | | | | | | | (Schizosaccharomyces pombe) |
| | | | | | | | >gi\|7491051\|pir\|\|T38550 hypothetical protein |
| | | | | | | | SPAC2F7.02c - fission yeast (Schizosaccharomyces |
| | | | | | | | pombe) |
| 2824 | LIB3602-022-Q6-K1-A4 | 119002 | BLASTX | 247 | 7.00E−21 | 41 | GLUCAN ENDO-1,3-BETA-GLUCOSIDASE A1 |
| | | | | | | | PRECURSOR ((1->3)-BETA-GLUCAN |
| | | | | | | | ENDOHYDROLASE) ((1->3)-BETA- |
| | | | | | | | GLUCANASE A1) >gi\|80085\|pir\|\|JQ0420 beta-1,3- |
| | | | | | | | glucanase A1 precursor - Bacillus circulans |
| | | | | | | | >gi\|142973\|gb\|AAA22474.1\| (M34503) beta-1,3- |
| | | | | | | | glucanase A1 (glcA) [Bacillu |
| 2825 | LIB3602-007-Q1-K1-D8 | 1169128 | BLASTX | 160 | 1.00E−10 | 38 | SERINE/THREONINE-PROTEIN KINASE CTR1 |
| | | | | | | | >gi\|166680\|gb\|AAA32779.1\| (L08789) protein |
| | | | | | | | kinase [Arabidopsis thaliana] |
| | | | | | | | >gi\|166682\|gb\|AAA32780.1\| (L08790) protein |
| | | | | | | | kinase [Arabidopsis thaliana] |
| | | | | | | | >gi\|7340658\|emb\|CAB82938.1\| (AL162506) |
| | | | | | | | SERINE/THREONINE-PROTEIN KINASE C |
| 2826 | LIB3602-103-Q1-KI-E11 | 7298660 | BLASTX | 213 | 8.00E−17 | 45 | (AE003665) CG10756 gene product [Drosophila |
| | | | | | | | melanogaster] |
| 2827 | LIB3602-092-Q6-K6-B7 | 7436158 | BLASTX | 246 | 2.00E−20 | 38 | probable H+-transporting ATP synthase (EC |
| | | | | | | | 3.6.1.34) gamma chain, mitochondrial - Arabidopsis |
| | | | | | | | thaliana |
| 2828 | LIB3602-007-Q1-K1-F5 | 7433647 | BLASTX | 567 | 2.00E−58 | 69 | acetyl-CoA C-acyltransferase (EC 2.3.1.16) |
| | | | | | | | precursor - cucurbit >gi\|1694621\|dbj\|BAA11117.1\| |
| | | | | | | | (D70895) 3-ketoacyl-CoA thiolase [Cucurbita sp.] |
| 2829 | LIB3602-111-Q1-K1-B1 | 6433784 | BLASTX | 257 | 6.00E−22 | 36 | (Y09633) hypothetical protein [Bradyrhizobium |
| | | | | | | | japonicum] |
| 2830 | LIB3602-032-Q6-K1-B10 | 5902586 | BLASTX | 167 | 2.00E−11 | 49 | (AF110781) photosystem I reaction center subunit |
| | | | | | | | PSAN precursor [Volvox carteri f. nagariensis] |
| 2831 | LIB3602-059-Q6-K1-C2 | 2498077 | BLASTX | 167 | 9.00E−12 | 54 | NUCLEOSIDE DIPHOSPHATE KINASE I (NDKI) |
| | | | | | | | (NDP KINASE I) (PP18) |
| | | | | | | | >gi\|1777930\|gb\|AAB40609.1\| (U55019) nucleoside |
| | | | | | | | diphosphate kinase [Saccharum officinarum] |
| 2832 | LIB3602-014-Q6-K1-F11 | 7488451 | BLASTX | 216 | 5.00E−17 | 52 | G box-binding protein homolog GBF1 - rape |
| | | | | | | | >gi\|1399007\|gb\|AAB03379.1\| (U27108) |
| | | | | | | | transcription factor [Brassica napus] |
| 2833 | LIB3602-055-Q6-K1-F1 | 400578 | BLASTX | 216 | 3.00E−17 | 45 | NADH-UBIQUINONE OXIDOREDUCTASE 18 KD |
| | | | | | | | SUBUNIT PRECURSOR (COMPLEX I-18 KD) |
| | | | | | | | (CI-18 KD) (COMPLEX I-AQDQ) (CI- |
| | | | | | | | AQDQ) >gi\|346531\|pir\|\|S28240 NADH |
| | | | | | | | dehydrogenase (ubiquinone) (EC 1.6.5.3) chain CI- |
| | | | | | | | 18(IP) precursor - bovine |
| | | | | | | | >gi\|226\|emb\|CAA44900.1\|(X63215) NADH deh |
| 2834 | LIB3602-057-Q6-K1-F6 | 7226932 | BLASTX | 266 | 5.00E−23 | 50 | (AE002518) tRNA (uracil-5-)-methyltransferase |
| | | | | | | | [Neisseria meningitidis] |
| 2835 | LIB3602-077-Q6-K6-F7 | 5326994 | BLASTX | 310 | 4.00E−28 | 67 | (AJ223326) DNA topoisomerase I [Daucus carota] |
| 2836 | LIB3602-055-Q6-K1-G10 | 6862961 | BLASTX | 357 | 1.00E−33 | 70 | (AC019018) putative deoxyoctulonosic acid |
| | | | | | | | synthetase [Arabidopsis thaliana] |
| 2837 | LIB3602-045-Q6-K1-C6 | 114718 | BLASTX | 179 | 7.00E−13 | 61 | PROBABLE COPPER-TRANSPORTING |
| | | | | | | | ATPASE SYNA >gi\|79650\|pir\|\|S10839 hypothetical |
| | | | | | | | protein 5 - Synechococcus sp. (PCC 6301) |
| | | | | | | | (fragment) >gi\|48026\|emb\|CAA29364.1\| (X05925) |
| | | | | | | | VRF 5 (293 AA) [Synechococcus PCC6301] |
| 2838 | LIB3602-050-Q6-K1-G1 | 6598335 | BLASTX | 234 | 2.00E−19 | 46 | (AC002329) putative glucanase [Arabidopsis |
| | | | | | | | thaliana] |
| 2839 | LIB3602-092-Q6-K6-H11 | 4662633 | BLASTX | 236 | 2.00E−19 | 35 | (AC007267) hypothetical protein [Arabidopsis |
| | | | | | | | thaliana] |
| 2840 | LIB3602-062-Q6-K1-B6 | 4510386 | BLASTX | 262 | 1.00E−22 | 53 | (AC007017) unknown protein [Arabidopsis |
| | | | | | | | thaliana] |
| 2841 | LIB3602-029-Q6-K1-F1 | 6706420 | BLASTX | 710 | 4.00E−75 | 80 | (AL133248) 40S ribosomal protein S2 homolog |
| | | | | | | | [Arabidopsis thaliana] |
| 2842 | LIB3602-044-Q6-K1-B8 | 6587806 | BLASTX | 187 | 8.00E−14 | 40 | (AC010924) Contains similarity to gb\|M82916 |
| | | | | | | | MRS2 protein from Saccharomyces cerivisae. ESTs |
| | | | | | | | gb\|N96043, gb\|AI998651, gb\|AA585850, |
| | | | | | | | gb\|T42027 come from this gene. [Arabidopsis |
| | | | | | | | thaliana] |
| 2843 | LIB3602-037-Q6-K1-A6 | 1076668 | BLASTX | 418 | 5.00E−41 | 75 | NADH dehydrogenase (ubiquinone) (EC 1.6.5.3) |
| | | | | | | | flavoprotein 1 precursor - potato |
| | | | | | | | >gi\|639834\|emb\|CAA58823.1\| (X83999) NADH |
| | | | | | | | dehydrogenase [Solanum tuberosum] |
| 2844 | LIB3602-080-Q6-K6-F2 | 3024121 | BLASTX | 147 | 2.00E−09 | 60 | S-ADENOSYLMETHIONINE SYNTHETASE |
| | | | | | | | (METHIONINE ADENOSYLTRANSFERASE) |
| | | | | | | | (ADOMET SYNTHETASE) |
| | | | | | | | >gi\|1724104\|gb\|AAB38500.1\| (U79767) methionine |
| | | | | | | | adenosyltransferase [Mesembryanthemum |
| | | | | | | | crystallinum] |
| 2845 | LIB3602-058-Q6-K1-D12 | 7300654 | BLASTN | 33 | 6.00E−09 | 90 | Drosophila melanogaster genomic scaffold |
| | | | | | | | 142000013386035 section 57 of 105, complete |
| | | | | | | | sequence |
| 2846 | LIB3602-077-Q6-K6-D3 | 3334490 | BLASTX | 173 | 2.00E−12 | 66 | DNA-DIRECTED RNA POLYMERASES I, II, |
| | | | | | | | AND III 7.3 KD POLYPEPTIDE (ABC10-ALPHA) |
| | | | | | | | >gi\|7490493\|pir\|\|T39794 DNA-directed RNA |
| | | | | | | | polymerases i, ii, and iii 7.3 kd polypeptide(abc10- |
| | | | | | | | alpha) - fission yeast (Schizosaccharomyces pombe) |
| | | | | | | | >gi\|2529253\|dbj\|BAA22807.1\| (D8963 |
| 2847 | LIB3602-111-Q1-K1-A6 | 7447234 | BLASTX | 181 | 5.00E−13 | 77 | probable iron-sulfur cofactor synthesis protein |
| | | | | | | | F6H11.150 - Arabidopsis thaliana |
| | | | | | | | >gi\|2827713\|emb\|CAA16686.1\| (AL021684) |
| | | | | | | | pyridoxal-phosphate-dependent aminotransferase - |
| | | | | | | | like protein [Arabidopsis thaliana] |
| | | | | | | | >gi\|6686815\|emb\|CAB64727.1\| (AJ243393) nifS- |
| | | | | | | | like p |
| 2848 | LIB3602-057-Q6-K1-E3 | 7437271 | BLASTX | 396 | 4.00E−38 | 64 | UDPglucose 4-epimerase (EC 5.1.3.2) (clone |
| | | | | | | | GEPI42) - guar >gi\|3021355\|emb\|CAA06338.1\| |
| | | | | | | | (AJ005081) UDP-galactose 4-epimerase |
| | | | | | | | [Cyamopsis tetragonoloba] |
| 2849 | LIB3602-030-Q6-K1-A10 | 2982272 | BLASTX | 256 | 6.00E−22 | 44 | (AF051220) hypothetical protein [Picea mariana] |
| 2850 | LIB3602-054-Q6-K1-G10 | 6735326 | BLASTX | 377 | 4.00E−36 | 70 | (AL137081) phenylalanine-tRNA synthetase-like |
| | | | | | | | protein [Arabidopsis thaliana] |
| 2851 | LIB3602-108-Q1-K1-C11 | 322386 | BLASTX | 158 | 1.00E−10 | 69 | chlorophyll a/b-binding protein - green alga |
| | | | | | | | (Pyrobotrys stellata) >gi\|18254\|emb\|CAA49209.1\| |
| | | | | | | | (X69434) a/b binding protein [Pyrobotrys stellata] |
| 2852 | LIB3602-065-Q1-K6-A2 | 2661048 | BLASTX | 147 | 5.00E−09 | 36 | (AF035293) similar to lysophospholipase [Homo |
| | | | | | | | sapiens] |
| 2853 | LIB3602-114-Q1-K1-E11 | 1345933 | BLASTX | 350 | 2.00E−34 | 69 | CITRATE SYNTHASE, GLYOXYSOMAL |
| | | | | | | | PRECURSOR (GCS) >gi\|1084323\|pir\|\|S53007 |
| | | | | | | | citrate synthase - cucurbit |
| | | | | | | | >gi\|975633\|dbj\|BAA07328.1\| (D38132) |
| | | | | | | | glyoxysomal citrate synthase [Cucurbita sp.] |
| 2854 | LIB3602-021-Q6-K1-G6 | 4139218 | BLASTX | 319 | 1.00E−29 | 68 | (AF104631) light harvesting complex II protein |
| | | | | | | | precursor [Chlamydomonas reinhardtii] |
| 2855 | LIB3602-114-Q1-K1-D5 | 2499503 | BLASTX | 478 | 1.00E−48 | 63 | PHOSPHOGLYCERATE KINASE |
| 2856 | LIB3602-103-Q1-K1-A5 | 7506763 | BLASTX | 350 | 2.00E−45 | 72 | hypothetical protein R166.2 - Caenorhabditis |
| | | | | | | | elegans >gi\|3879192\|emb\|CAA90663.1\| (Z50795) |
| | | | | | | | weak similarity with yeast cat8 regulatory protein |
| | | | | | | | (Swiss Prot accession number P39113); cDNA EST |
| | | | | | | | EMBL: Z14554 comes from this gene; cDNA EST |
| | | | | | | | EMBL: T02057 comes from t |
| 2857 | LIB3602-103-Q1-K1-A10 | 4389317 | BLASTX | 226 | 1.00E−18 | 66 | Chain A, Nucleoside Diphosphate Kinase Isoform B |
| | | | | | | | From Bovine Retina >gi\|4389318\|pdb\|1BE4\|B |
| | | | | | | | Chain B, Nucleoside Diphosphate Kinase Isoform B |
| | | | | | | | From Bovine Retina |
| 2858 | LIB3602-027-Q6-K1-A5 | 4914683 | BLASTX | 239 | 8.00E−20 | 33 | (AF067728) transactivating protein BRIDGE |
| | | | | | | | [Rattus norvegicus] |
| 2859 | LIB3602-046-Q6-K1-A7 | 7473075 | BLASTX | 305 | 4.00E−28 | 62 | isocitrate lyase - Deinococcus radiodurans (strain |
| | | | | | | | R1) >gi\|6458545\|gb\|AAF10407.1\|AE001937_3 |
| | | | | | | | (AE001937) isocitrate lyase [Deinococcus |
| | | | | | | | radiodurans] |
| 2860 | LIB3602-057-Q6-K1-D3 | 7436714 | BLASTX | 524 | 3.00E−53 | 69 | acetolactate synthase (EC 4.1.3.18) - |
| | | | | | | | Chlamydomonas reinhardtii |
| | | | | | | | >gi\|2906139\|gb\|AAC03784.1\| (AF047459) |
| | | | | | | | acetolactate synthase [Chlamydomonas reinhardtii] |
| 2861 | LIB3602-101-Q1-K1-H11 | 3882355 | BLASTX | 159 | 1.00E−10 | 72 | (U92460) 12-oxophytodienoate reductase OPR1 |
| | | | | | | | [Arabidopsis thaliana] |
| | | | | | | | >gi\|6143902\|gb\|AAF04448.1\|AC010718_17 |
| | | | | | | | (AC010718) 12-oxophytodienoate reductase |
| | | | | | | | (OPR1) [Arabidopsis thaliana] |
| 2862 | LIB3602-011-Q6-K1-H11 | 6648019 | BLASTX | 301 | 4.00E−27 | 39 | HYPOTHETICAL 45.2 KD PROTEIN RV2685 |
| | | | | | | | >gi\|7477890\|pir\|\|H70528 probable arsB protein - |
| | | | | | | | Mycobacterium tuberculosis (strain H37RV) |
| | | | | | | | >gi\|2181976\|emb\|CAB09476.1\| (Z96072) arsB |
| | | | | | | | [Mycobacterium tuberculosis] |
| 2863 | LIB3602-055-Q6-K1-E4 | 7362763 | BLASTX | 401 | 7.00E−39 | 57 | (AL162651) dTDP-glucose 4-6-dehydratase |
| | | | | | | | homolog D18 [Arabidopsis thaliana] |
| 2864 | LIB3602-115-Q1-K1-F6 | 7488693 | BLASTX | 545 | 1.00E−55 | 65 | phosphate transport protein G7, mitochondrial - |
| | | | | | | | soybean >gi\|3318611\|dbj\|BAA31582.1\| |
| | | | | | | | (AB016063) mitochondrial phosphate transporter |
| | | | | | | | [Glycine max] |
| 2865 | LIB3602-047-Q6-K1-E11 | 2500074 | BLASTX | 757 | 1.00E−80 | 89 | GTP-BINDING PROTEIN YPTC4 |
| | | | | | | | >gi\|1363474\|pir\|\|JC4106 GTP-binding protein |
| | | | | | | | yptC4 - Chlamydomonas reinhardtii |
| | | | | | | | >gi\|806722\|gb\|AAA82726.1\| (U13167) YptC4 |
| | | | | | | | [Chlamydomonas reinhardtii] |
| 2866 | LIB3602-077-Q6-K6-E11 | 6686798 | BLASTX | 242 | 4.00E−20 | 43 | (AJ242481) FKBP like protein [Arabidopsis |
| | | | | | | | thaliana] |
| 2867 | LIB3602-028-Q6-K1-D2 | 281003 | BLASTX | 148 | 3.00E−09 | 38 | T-complex protein 10c (C3H allele) - mouse |
| 2868 | LIB3602-055-Q6-K1-E9 | 3962377 | BLASTX | 480 | 4.00E−48 | 57 | (AJ002551) heat shock protein 70 [Arabidopsis |
| | | | | | | | thaliana] |
| 2869 | LIB3602-064-Q1-K6-E5 | 2829916 | BLASTX | 160 | 2.00E−10 | 51 | (AC002291) Unknown protein [Arabidopsis |
| | | | | | | | thaliana] |
| 2870 | LIB3602-004-Q1-K1-G11 | 7441896 | BLASTX | 415 | 2.00E−40 | 54 | heat shock protein 90 homolog T22A6.20 - |
| | | | | | | | Arabidopsis thaliana |
| | | | | | | | >gi\|5051761\|emb\|CAB45054.1\| (AL078637) |
| | | | | | | | HSP90-like protein [Arabidopsis thaliana] |
| | | | | | | | >gi\|7269269\|emb\|CAB79329.1\| (AL161561) |
| | | | | | | | HSP90-like protein [Arabidopsis thaliana] |
| 2871 | LIB3602-063-Q1-K6-B1 | 7487954 | BLASTX | 155 | 4.00E−10 | 65 | microfibril-associated protein homolog T15F16.8 - |
| | | | | | | | Arabidopsis thaliana |
| | | | | | | | >gi\|2565010\|gb\|AAB81880.1\|AAB81880 |
| | | | | | | | (AC002983) putative microfibril-associated protein |
| | | | | | | | [Arabidopsis thaliana] |
| | | | | | | | >gi\|3377811\|gb\|AAC28184.1\| (AF076275) contains |
| | | | | | | | similarity to ATP synthase B |
| 2872 | LIB3602-019-Q6-K1-C6 | 2267084 | BLASTX | 149 | 3.00E−15 | 72 | (AF007889) calmodulin [Symbiodinium |
| | | | | | | | microadriaticum] |
| 2873 | LIB3602-068-Q1-K1-A2 | 7471620 | BLASTX | 185 | 2.00E−13 | 41 | conserved hypothetical protein - Deinococcus |
| | | | | | | | radiodurans (strain R1) |
| | | | | | | | >gi\|6458247\|gb\|AAF10133.1\|AE001913_7 |
| | | | | | | | (AE001913) conserved hypothetical protein |
| | | | | | | | [Deinococcus radiodurans] |
| 2874 | LIB3602-088-Q6-K6-H1 | 4416330 | BLASTX | 305 | 1.00E−27 | 70 | (AF105295) S-adenosyl-homocysteine hydrolase |
| | | | | | | | like protein; SAHH-like protein [Alexandrium |
| | | | | | | | fundyense] |
| 2875 | LIB3602-003-Q1-K1-A10 | 464849 | BLASTX | 872 | 5.00E−94 | 93 | TUBULIN ALPHA CHAIN >gi\|486847\|pir\|\|S36232 |
| | | | | | | | tubulin alpha chain - almond |
| | | | | | | | >gi\|20413\|emb\|CAA47635.1\| (X67162) alpha- |
| | | | | | | | tubulin [Prunus dulcis] |
| 2876 | LIB3602-108-Q1-K1-F2 | 461999 | BLASTX | 587 | 8.00E−61 | 81 | ELONGATION FACTOR G, CHLOROPLAST |
| | | | | | | | PRECURSOR (EF-G) |
| 2877 | LIB3602-076-Q6-K6-H11 | 2982245 | BLASTX | 276 | 4.00E−24 | 49 | (AF051205) hypothetical protein [Picea mariana] |
| 2878 | LIB3602-029-Q6-K1-C6 | 322525 | BLASTX | 610 | 2.00E−63 | 69 | omnipotent suppressor protein SUP1 homolog |
| | | | | | | | (clone A18) - Arabidopsis thaliana (fragment) |
| | | | | | | | >gi\|16512\|emb\|CAA49171.1\| (X69374) similar to |
| | | | | | | | yeast omnipotent suppressor protein SUP1 |
| | | | | | | | (SUP45); ORF [Arabidopsis thaliana] |
| 2879 | LIB3602-068-Q1-K1-G12 | 7327817 | BLASTX | 171 | 8.00E−12 | 32 | (AL161946) putative protein [Arabidopsis thaliana] |
| 2880 | LIB3602-007-Q1-K1-G3 | 6651387 | BLASTN | 41 | 7.00E−14 | 84 | Cloning vector pDDB57 complete sequence |
| 2881 | LIB3602-104-Q1-K1-B10 | 7488813 | BLASTX | 278 | 2.00E−24 | 33 | import intermediate-associated 100K protein |
| | | | | | | | precursor - garden pea |
| | | | | | | | >gi\|1495768\|emb\|CAA92823.1\| (Z68506) |
| | | | | | | | chloroplast inner envelope protein, 110 kD |
| | | | | | | | (IEP110) [Pisum sativum] |
| 2882 | LIB3602-050-Q6-K1-C11 | 4375938 | BLASTX | 178 | 9.00E−13 | 44 | (AL031055) dH28H20.1 (similar to membrane |
| | | | | | | | transport protein) [Homo sapiens] |
| 2883 | LIB3602-081-Q6-K6-F7 | 6677681 | BLASTX | 280 | 2.00E−47 | 52 | retinoblastoma binding protein 4 |
| | | | | | | | >gi\|2494893\|sp\|Q60972\|RB48_MOUSE |
| | | | | | | | CHROMATIN ASSEMBLY FACTOR 1 P48 |
| | | | | | | | SUBUNIT (CAF-1 P48 SUBUNIT) |
| | | | | | | | (RETINOBLASTOMA BINDING PROTEIN P48) |
| | | | | | | | (RETINOBLASTOMA-BINDING PROTEIN 4) |
| | | | | | | | >gi\|2137734\|pir\|\|I49366 G1/S transition control |
| | | | | | | | protein-b |
| 2884 | LIB3602-120-Q1-K1-D4 | 3915851 | BLASTX | 202 | 1.00E−15 | 61 | CHLOROPLAST 30S RIBOSOMAL PROTEIN |
| | | | | | | | S10 >gi\|3603070\|gb\|AAC35731.1\| (AF041468) |
| | | | | | | | ribosomal protein S10 [Guillardia theta] |
| 2885 | LIB3602-020-Q6-K1-F3 | 6911549 | BLASTX | 517 | 1.00E−52 | 87 | (AJ249329) heat shock protein 70 [Cucumis |
| | | | | | | | sativus] |
| 2886 | LIB3602-054-Q6-K1-A4 | 4741966 | BLASTX | 206 | 5.00E−16 | 54 | (AF134133) Li13 protein [Arabidopsis thaliana] |
| 2887 | LIB3602-007-Q1-K1-G5 | 7302240 | BLASTX | 344 | 5.00E−32 | 49 | (AE003787) CG10417 gene product [Drosophila |
| | | | | | | | melanogaster] |
| 2888 | LIB3602-051-Q6-K1-G12 | 7304024 | BLASTX | 178 | 1.00E−12 | 41 | (AE003836) CG14750 gene product [Drosophila |
| | | | | | | | melanogaster] |
| 2889 | LIB3602-011-Q6-K1-H1 | 1174844 | BLASTX | 278 | 2.00E−24 | 53 | UBIQUITIN-CONJUGATING ENZYME E2-17 |
| | | | | | | | KD 2 (UBIQUITIN-PROTEIN LIGASE 2) |
| | | | | | | | (UBIQUITIN CARRIER PROTEIN 2) |
| | | | | | | | >gi\|1076426\|pir\|\|S43783 ubiquitin-conjugating |
| | | | | | | | enzyme UBC2 - Arabidopsis thaliana |
| | | | | | | | >gi\|431264\|gb\|AAA32899.1\| (L19353) ubiquitin |
| | | | | | | | conjugating enzyme [Arabidopsi |
| 2890 | LIB3602-079-Q6-K6-H2 | 7542542 | BLASTX | 221 | 1.00E−17 | 91 | (AF238866) LNR42 [Mus musculus] |
| 2891 | LIB3602-035-Q1-K1-B8 | 7301359 | BLASTX | 145 | 7.00E−09 | 41 | (AE003752) BcDNA: LD22548 gene product |
| | | | | | | | [Drosophila melanogaster] |
| 2892 | LIB3602-107-Q1-K1-E10 | 1346802 | BLASTX | 444 | 8.00E−44 | 60 | PROFILIN 1 >gi\|1076516\|pir\|\|S49351 profilin 1 - |
| | | | | | | | kidney bean >gi\|556836\|emb\|CAA57508.1\| |
| | | | | | | | (X81982) profilin [Phaseolus vulgaris] |
| 2893 | LIB3602-102-Q1-K1-D6 | 135099 | BLASTX | 447 | 2.00E−51 | 65 | ASPARTYL-TRNA SYNTHETASE |
| | | | | | | | (ASPARTATE--TRNA LIGASE) (ASPRS) |
| | | | | | | | >gi\|68531\|pir\|\|SYRTDT aspartate-tRNA ligase (EC |
| | | | | | | | 6.1.1.12) - rat >gi\|203066\|gb\|AAA40789.1\| |
| | | | | | | | (J04487) aspartyl-tRNA synthetase [Rattus |
| | | | | | | | norvegicus] >gi\|1773257\|gb\|AAC52981.1\| |
| | | | | | | | (U30812) aspartyl-tRNA sy |
| 2894 | LIB3602-039-Q6-K1-A2 | 6437556 | BLASTX | 343 | 6.00E−32 | 38 | (AC011623) unknown protein [Arabidopsis |
| | | | | | | | thaliana] |
| 2895 | LIB3602-027-Q6-K1-E11 | 3915866 | BLASTX | 404 | 3.00E−39 | 54 | GLUTAMINYL-TRNA SYNTHETASE |
| | | | | | | | (GLUTAMINE- TRNA LIGASE) (GLNRS) |
| | | | | | | | >gi\|7437754\|pir\|\|T09643 glutamine- tRNA ligase |
| | | | | | | | (EC 6.1.1.18) - yellow lupine |
| | | | | | | | >gi\|2995455\|emb\|CAA62901.1\| (X91787) tRNA- |
| | | | | | | | glutamine synthetase [Lupinus luteus] |
| 2896 | LIB3602-085-Q6-K1-B11 | 132917 | BLASTX | 189 | 2.00E−16 | 52 | 60S RIBOSOMAL PROTEIN L35 |
| | | | | | | | >gi\|71362\|pir\|\|R5RT35 ribosomal protein L35 - rat |
| | | | | | | | >gi\|57702\|emb\|CAA36001.1\| (X51705) ribosomal |
| | | | | | | | protein L35 (AA 1-123) [Rattus rattus] |
| 2897 | LIB3602-036-Q6-K1-E6 | 3287875 | BLASTX | 170 | 1.00E−11 | 40 | PROBABLE TETRAACYLDISACCHARIDE 4′- |
| | | | | | | | KINASE (LIPID A 4′-KINASE) |
| | | | | | | | >gi\|290802\|gb\|AAD15238.1\| (L17003) valB gene |
| | | | | | | | product [Francisella tularensis var. novicida] |
| 2898 | LIB3602-038-Q6-K1-E10 | 115827 | BLASTX | 336 | 3.00E−31 | 80 | CHLOROPHYLL A-B BINDING PROTEIN OF |
| | | | | | | | LHCII TYPE I PRECURSOR (CAB) (LHCP) |
| | | | | | | | >gi\|99383\|pir\|\|A31392 chlorophyll a/b-binding |
| | | | | | | | protein - Chlamydomonas reinhardtii |
| | | | | | | | >gi\|167409\|gb\|AAA33082.1\| (M24072) chlorophyll |
| | | | | | | | a/b-binding protein [Chlamydomonas reinhardtii] |
| 2899 | LIB3602-065-Q1-K6-B10 | 7441879 | BLASTX | 627 | 2.00E−65 | 79 | dnaK-type molecular chaperone HSC70-11, |
| | | | | | | | mitochondrial - spinach |
| | | | | | | | >gi\|2654212\|gb\|AAB91473.1\| (AF035458) heat |
| | | | | | | | shock 70 protein [Spinacia oleracea] |
| | | | | | | | >gi\|2773052\|gb\|AAB96660.1\| (AF039084) heat |
| | | | | | | | shock 70 protein [Spinacia oleracea] |
| 2900 | LIB3602-016-Q6-K1-B10 | 7489392 | BLASTX | 216 | 3.00E−17 | 45 | N-carbamyl-L-amino acid amidohydrolase homolog - |
| | | | | | | | barley (fragment) >gi\|2695925\|emb\|CAA10981.1\| |
| | | | | | | | (AJ222776) hypothetical protein [Hordeum vulgare] |
| 2901 | LIB3602-036-Q6-K1-F2 | 1177368 | BLASTN | 33 | 5.00E−09 | 87 | Z. mays mRNA for ribosomal protein L39 |
| 2902 | LIB3602-102-Q1-K1-G6 | 4056502 | BLASTX | 183 | 2.00E−13 | 55 | (AC005896) 40S ribosomal protein S5 [Arabidopsis |
| | | | | | | | thaliana] |
| 2903 | LIB3602-077-Q6-K6-C9 | 7301524 | BLASTX | 631 | 1.00E−65 | 61 | (AE003758) CG6309 gene product [Drosophila |
| | | | | | | | melanogaster] |
| 2904 | LIB3602-017-Q6-K1-E5 | 6681113 | BLASTX | 259 | 3.00E−22 | 36 | cytochrome P450, steroid inducible 3a11 |
| | | | | | | | >gi\|5921911\|sp\|Q64459\|CP3B_MOUSE |
| | | | | | | | CYTOCHROME P450 3A11 (CYPIIIA11) (P- |
| | | | | | | | 450IIIAM1) (P-450UT) >gi\|2117381\|pir\|\|A60564 |
| | | | | | | | cytochrome P450 3A11 - mouse |
| | | | | | | | >gi\|50535\|emb\|CAA42981.1\| (X60452) cytochrome |
| | | | | | | | P-450IIIA [Mus musculus] |
| 2905 | LIB3602-118-Q1-K1-A10 | 6016708 | BLASTX | 216 | 5.00E−17 | 37 | (AC009325) putative protein kinase [Arabidopsis |
| | | | | | | | thaliana] |
| 2906 | LIB3602-078-Q6-K6-H1 | 5852164 | BLASTX | 192 | 2.00E−18 | 43 | (AJ249389) vacuolar ATPase subunit H [Manduca |
| | | | | | | | sexta] |
| 2907 | LIB3602-105-Q1-K1-G11 | 1174853 | BLASTX | 335 | 5.00E−31 | 48 | UBIQUITIN-CONJUGATING ENZYME E2-18 |
| | | | | | | | KD (UBIQUITIN-PROTEIN LIGASE) |
| | | | | | | | (UBIQUITIN CARRIER PROTEIN) (PM42) |
| | | | | | | | >gi\|481811\|pir\|\|S39483 ubiquitin-conjugating |
| | | | | | | | enzyme UBC2-1 - Arabidopsis thaliana |
| | | | | | | | >gi\|22658\|emb\|CAA48378.1\| (X68306) ubiquitin- |
| | | | | | | | conjugating enzyme [Arabidop |
| 2908 | LIB3602-107-Q1-K1-H4 | 3478700 | BLASTX | 236 | 2.00E−19 | 39 | (AF034387) AFT protein [Arabidopsis thaliana] |
| 2909 | LIB3602-061-Q6-K1-B9 | 2791900 | BLASTX | 213 | 8.00E−17 | 47 | (AJ000057) PP7 [Arabidopsis thaliana] |
| 2910 | LIB3602-032-Q6-K1-F2 | 1945479 | BLASTX | 174 | 2.00E−12 | 65 | (U96178) fructose-bisphosphate aldolase |
| | | | | | | | [Onchocerca volvulus] |
| 2911 | LIB3602-102-Q1-K1-H2 | 7513277 | BLASTX | 550 | 3.00E−56 | 63 | probable transmembrane protein TMC - human |
| 2912 | LIB3602-105-Q1-K1-G4 | 6137575 | BLASTX | 185 | 2.00E−13 | 47 | Chain A, Structure And Characterization Of |
| | | | | | | | Ectothiorhodospira Vacuolata Cytochrome B558, A |
| | | | | | | | Prokaryotic Homologue Of Cytochrome B5 |
| | | | | | | | >gi\|5919159\|gb\|AAD56233.1\| (AF183259) |
| | | | | | | | cytochrome b558 [Ectothiorhodospira vacuolata] |
| 2913 | LIB3602-001-P1-K6-E3 | 7449902 | BLASTX | 172 | 2.00E−12 | 58 | probable 12-oxophytodienoate reductase (EC |
| | | | | | | | 1.3.1.42) CPRD8, drought-inducible - cowpea |
| | | | | | | | >gi\|1854443\|dbj\|BAA12160.1\| (D83970) CPRD8 |
| | | | | | | | protein [Vigna unguiculata] |
| 2914 | LIB3602-025-Q6-K1-E7 | 7019666 | BLASTX | 507 | 3.00E−51 | 58 | (AL132954) putative protein [Arabidopsis thaliana] |
| 2915 | LIB3602-062-Q6-K1-G9 | 7485705 | BLASTX | 421 | 4.00E−41 | 50 | hypothetical protein F18B3.240 - Arabidopsis |
| | | | | | | | thaliana >gi\|4835247\|emb\|CAB42925.1\| |
| | | | | | | | (AL049862) putative tRNA synthetase [Arabidopsis |
| | | | | | | | thaliana] |
| 2916 | LIB3602-047-Q6-K1-G10 | 7486272 | BLASTX | 532 | 3.00E−54 | 62 | hypothetical protein F28A21.220 - Arabidopsis |
| | | | | | | | thaliana >gi\|4539400\|emb\|CAB37466.1\| |
| | | | | | | | (AL035526) putative protein [Arabidopsis thaliana] |
| | | | | | | | >gi\|7268675\|emb\|CAB78883.1\| (AL161549) |
| | | | | | | | putative protein [Arabidopsis thaliana] |
| 2917 | LIB3602-018-Q6-K1-B10 | 7486436 | BLASTX | 174 | 3.00E−12 | 28 | hypothetical protein F4I1.34 - Arabidopsis thaliana |
| 2918 | LIB3602-058-Q6-K1-H8 | 6630456 | BLASTX | 298 | 8.00E−27 | 45 | (AC007190) F23N19.15 [Arabidopsis thaliana] |
| 2919 | LIB3602-064-Q1-K6-D1 | 730456 | BLASTX | 218 | 2.00E−17 | 35 | 40S RIBOSOMAL PROTEIN S19 |
| 2920 | LIB3602-049-Q6-K1-G3 | 7259797 | BLASTX | 152 | 1.00E−09 | 29 | (AF216743) epsilon tubulin [Trypanosoma brucei] |
| 2921 | LIB3602-090-Q6-K6-C2 | 683699 | BLASTX | 151 | 2.00E−09 | 58 | (Z48229) orf2 [Saccharomyces cerevisiae] |
| 2922 | LIB3602-015-Q6-K1-B7 | 5453603 | BLASTX | 735 | 6.00E−78 | 70 | chaperonin containing TCP1, subunit 2 (beta) |
| | | | | | | | >gi\|6094436\|sp\|P78371\|TCPB_HUMAN T- |
| | | | | | | | COMPLEX PROTEIN 1, BETA SUBUNIT (TCP- |
| | | | | | | | 1-BETA) (CCT-BETA) |
| | | | | | | | >gi\|2559012\|gb\|AAC96012.1\| (AF026293) |
| | | | | | | | chaperonin containing t-complex polypeptide 1, |
| | | | | | | | beta subunit; CCT-beta [Homo sapie |
| 2923 | LIB3602-015-Q6-K1-B3 | 4105639 | BLASTX | 367 | 8.00E−35 | 74 | (AF049356) phytoene desaturase precursor [Oryza |
| | | | | | | | sativa] |
| 2924 | LIB3602-019-Q6-K1-G1 | 7433407 | BLASTX | 481 | 3.00E−48 | 79 | nifU protein homolog T24H24.11 - Arabidopsis |
| | | | | | | | thaliana >gi\|3377840\|gb\|AAC28213.1\| (AF075598) |
| | | | | | | | contains similarity to E. coli nitrogen fixation NIFU |
| | | | | | | | protein (GB: AE000339) [Arabidopsis thaliana] |
| | | | | | | | >gi\|7267164\|emb\|CAB77876.1\| (AL161499) |
| | | | | | | | putative NifU-like metall |
| 2925 | LIB3602-043-Q6-K1-C1 | 140161 | BLASTX | 172 | 4.00E−12 | 36 | HYPOTHETICAL 20.1 KD PROTEIN IN MOG- |
| | | | | | | | HTGA INTERGENIC REGION (ORF5) |
| | | | | | | | >gi\|1073400\|pir\|\|E56688 protein yaaH - Escherichia |
| | | | | | | | coli >gi\|41756\|emb\|CAA47931.1\| (X67700) ORF5 |
| | | | | | | | [Escherichia coli] >gi\|1786191\|gb\|AAC73121.1\| |
| | | | | | | | (AE000111) orf, hypothetical protein [Escherich |
| 2926 | -LIB3602-045-Q6-K1-F1 | 7491946 | BLASTX | 165 | 3.00E−11 | 41 | hypothetical protein SPCC622.19 - fission yeast |
| | | | | | | | (Schizosaccharomyces pombe) |
| | | | | | | | >gi\|4539271\|emb\|CAA21875.2\| (AL033127) |
| | | | | | | | hypothetical protein [Schizosaccharomyces pombe] |
| 2927 | LIB3602-039-Q6-K1-D4 | 7387549 | BLASTX | 600 | 3.00E−62 | 67 | ACETYLGLUTAMATE KINASE (NAG KINASE) |
| | | | | | | | (AGK) (N-ACETYL-L-GLUTAMATE 5- |
| | | | | | | | PHOSPHOTRANSFERASE) |
| | | | | | | | >gi\|7434572\|pir\|\|S77509 acetylglutamate kinase (EC |
| | | | | | | | 2.7.2.8) - Synechocystis sp. (strain PCC 6803) |
| | | | | | | | >gi\|1652434\|dbj\|BAA17356.1\| (D90905) N- |
| | | | | | | | acetylglutamate kinase [Synechoc |
| 2928 | LIB3602-113-Q1-K1-G12 | 3334201 | BLASTX | 223 | 7.00E−18 | 60 | GLYCINE CLEAVAGE SYSTEM H PROTEIN |
| | | | | | | | PRECURSOR >gi\|744170\|pir\|\|T12561 glycine |
| | | | | | | | cleavage system protein H precursor - common ice |
| | | | | | | | plant >gi\|1724106\|gb\|AAB38501.1\| (U79768) |
| | | | | | | | glycine cleavage system protein H precursor |
| | | | | | | | [Mesembryanthemum crystallinum] |
| 2929 | LIB3602-011-Q6-K1-B10 | 4006910 | BLASTX | 189 | 6.00E−14 | 44 | (Z99708) putative protein [Arabidopsis thaliana] |
| | | | | | | | >gi\|7270601\|emb\|CAB80319.1\| (AL161589) |
| | | | | | | | putative protein [Arabidopsis thaliana] |
| 2930 | LIB3602-111-Q1-K1-A11 | 7433421 | BLASTX | 254 | 1.00E−26 | 62 | 5-methyltetrahydrofolate--homocysteine S- |
| | | | | | | | methyltransferase (EC 2.1.1.13) - rat |
| | | | | | | | >gi\|3978143\|gb\|AAD05384.1\| (AF034214) |
| | | | | | | | methionine synthase; MS [Rattus norvegicus] |
| 2931 | LIB3602-071-Q1-K1-B6 | 6525170 | BLASTX | 515 | 2.00E−52 | 63 | (AF189278) ASF1 [Drosophila melanogaster] |
| | | | | | | | >gi\|7293763\|gb\|AAF49131.1\| (AE003516) asf1 |
| | | | | | | | gene product [Drosophila melanogaster] |
| 2932 | LIB3602-023-Q6-K1-G2 | 4104764 | BLASTX | 147 | 5.00E−09 | 50 | (AF039534) salicylate hydroxylase [Pseudomonas |
| | | | | | | | stutzeri] |
| 2933 | LIB3602-059-Q6-K1-G5 | 7434315 | BLASTX | 278 | 2.00E−24 | 44 | mitogen-activated protein kinase I (EC 2.7.1.—) - |
| | | | | | | | parsley >gi\|2231034\|emb\|CAA73323.1\| (Y12785) |
| | | | | | | | MAP kinase I [Petroselinum crispum] |
| 2934 | LIB3602-079-Q6-K6-F4 | 6692729 | BLASTX | 223 | 7.00E−18 | 46 | (AC012679) unknown protein [Arabidopsis |
| | | | | | | | thaliana] |
| 2935 | LIB3602-028-Q6-K1-H7 | 267147 | BLASTX | 336 | 3.00E−31 | 57 | DNA TOPOISOMERASE I >gi\|479841\|pir\|\|S35521 |
| | | | | | | | DNA topoisomerase (EC 5.99.1.2) I - fruit fly |
| | | | | | | | (Drosophila melanogaster) |
| | | | | | | | >gi\|158643\|gb\|AAA28951.1\| (M74557) |
| | | | | | | | topoisomerase I [Drosophila melanogaster] |
| | | | | | | | >gi\|1772834\|gb\|AAC24158.1\| (U80064) DNA |
| | | | | | | | topoisomerase I [Drosop |
| 2936 | LIB3602-056-Q6-K1-H3 | 7022048 | BLASTX | 182 | 3.00E−13 | 43 | (AK001028) unnamed protein product [Homo |
| | | | | | | | sapiens] |
| 2937 | LIB3602-103-Q1-K1-E8 | 7433412 | BLASTX | 462 | 5.00E−46 | 88 | nifU protein homolog TI0I14.50 - Arabidopsis |
| | | | | | | | thaliana >gi\|2832672\|emb\|CAA16772.1\| |
| | | | | | | | (AL021712) nifU-like protein [Arabidopsis |
| | | | | | | | thaliana] >gi\|7269067\|emb\|CAB79177.1\| |
| | | | | | | | (AL161556) nifU-like protein [Arabidopsis |
| | | | | | | | thaliana] |
| 2938 | LIB3602-027-Q6-K1-E1 | 1352458 | BLASTX | 471 | 5.00E−47 | 54 | INOSINE-5′-MONOPHOSPHATE |
| | | | | | | | DEHYDROGENASE (IMP DEHYDROGENASE) |
| | | | | | | | (IMPDH) (IMPD) >gi\|2117507\|pir\|\|JC4999 IMP |
| | | | | | | | dehydrogenase (EC 1.1.1.205) - Arabidopsis |
| | | | | | | | thaliana >gi\|1100063\|gb\|AAB41940.1\| (L34684) |
| | | | | | | | IMP dehydrogenase [Arabidopsis thaliana] |
| | | | | | | | >gi\|4835762\|gb\|AAD30229. |
| 2939 | LIB3602-095-Q6-K6-A4 | 7484827 | BLASTX | 228 | 2.00E−18 | 46 | brefeldin A-sensitive Golgi protein LDLC homolog |
| | | | | | | | F6I7.50 - Arabidopsis thaliana |
| | | | | | | | >gi\|4678263\|emb\|CAB41124.1\| (AL049657) |
| | | | | | | | brefeldin A-sensitive Golgi protein-like |
| | | | | | | | [Arabidopsis thaliana] |
| | | | | | | | >gi\|7269335\|emb\|CAB79394.1\| (AL161562) |
| | | | | | | | brefeldin A-sensitive Golgi prote |
| 2940 | LIB3602-057-Q6-K1-E8 | 7431479 | BLASTX | 287 | 4.00E−38 | 65 | methylmalonate-semialdehyde dehydrogenase |
| | | | | | | | (acylating) (EC 1.2.1.27) - fruit fly (Drosophila |
| | | | | | | | melanogaster) >gi\|2653397\|emb\|CAA15632.1\| |
| | | | | | | | (AL009147) /prediction = (method: ““genefinder””, |
| | | | | | | | version: ““084””, score: ““86.60””); |
| | | | | | | | /prediction = (method: ““genscan””, versio |
| 2941 | LIB3602-068-Q1-K1-H5 | 6175169 | BLASTX | 150 | 2.00E−09 | 42 | (AC011437) unknown protein [Arabidopsis |
| | | | | | | | thaliana] |
| 2942 | LIB3602-056-Q6-K1-D9 | 5902359 | BLASTX | 183 | 2.00E−13 | 40 | (AC009322) Heat-shock protein [Arabidopsis |
| | | | | | | | thaliana] |
| | | | | | | | >gi\|6453873\|gb\|AAF09057.1\|AC011717_24 |
| | | | | | | | (AC011717) putative heat-shock protein |
| | | | | | | | [Arabidopsis thaliana] |
| 2943 | LIB3602-071-Q1-K1-H11 | 7484809 | BLASTX | 264 | 1.00E−22 | 32 | ankyrin repeat-containing protein 2 - Arabidopsis |
| | | | | | | | thaliana >gi\|5830787\|emb\|CAB54873.1\| |
| | | | | | | | (AL117188) ankyrin repeat-containing protein 2 |
| | | | | | | | [Arabidopsis thaliana] |
| | | | | | | | >gi\|7270496\|emb\|CAB80261.1\| (AL161587) |
| | | | | | | | ankyrin repeat-containing protein 2 [Arabidopsis |
| | | | | | | | thaliana] |
| 2944 | LIB3602-004-Q1-K1-D10 | 4138880 | BLASTN | 38 | 1.00E−11 | 88 | Scenedesmus obliquus plastocyanin (Pcy) gene, |
| | | | | | | | Pcy-1 allele, complete cds |
| 2945 | LIB3602-049-Q6-K1-C1 | 4138881 | BLASTX | 309 | 5.00E−28 | 46 | (AF114235) plastocyanin [Scenedesmus obliquus] |
| 2946 | LIB3602-007-Q1-K1-G8 | 3461884 | BLASTX | 145 | 4.00E−09 | 61 | (AB006082) phosphoribosyl-ATP |
| | | | | | | | pyrophosphohydrolase [Arabidopsis thaliana] |
| | | | | | | | >gi\|3461886\|dbj\|BAA32529.1\| (AB006083) |
| | | | | | | | phosphoribosyl-ATP pyrophosphohydrolase |
| | | | | | | | [Arabidopsis thaliana] |
| 2947 | LIB3602-048-Q6-K1QA-A8 | 4966344 | BLASTX | 157 | 3.00E−10 | 44 | (AC006341) ESTs gb\|F15498, gb\|H37515, |
| | | | | | | | gb\|T41906, gb\|T22448, gb\|W43356 and gb\|T20739 |
| | | | | | | | come from this gene. [Arabidopsis thaliana] |
| 2948 | LIB3602-018-Q6-K1-F3 | 417540 | BLASTX | 449 | 2.00E−44 | 61 | PYRROLINE-5-CARBOXYLATE REDUCTASE |
| | | | | | | | (P5CR) (P5C REDUCTASE) |
| | | | | | | | >gi\|7431797\|pir\|\|T06477 probable pyrroline-5- |
| | | | | | | | carboxylate reductase (EC 1.5.1.2) - garden pea |
| | | | | | | | >gi\|20851\|emb\|CAA44646.1\| (X62842) pyrroline |
| | | | | | | | carboxylate reductase [Pisum sativum] |
| | | | | | | | >gi\|445614\|prf\|\|190936 |
| 2949 | LIB3602-046-Q6-K1-C5 | 7635782 | BLASTX | 280 | 1.00E−24 | 51 | (AL353819) 2,5-diketo-D-gluconic acid reductase |
| | | | | | | | related protein [Neurospora crassa] |
| 2950 | LIB3602-107-Q1-K1-F8 | 7487148 | BLASTX | 484 | 2.00E−48 | 59 | hypothetical protein T18E12.21 - Arabidopsis |
| | | | | | | | thaliana >gi\|3548818\|gb\|AAC34490.1\| (AC005313) |
| | | | | | | | unknown protein [Arabidopsis thaliana] |
| 2951 | LIB3602-102-Q1-K1-H4 | 7206593 | BLASTX | 280 | 6.00E−34 | 59 | (AC006608) similar to synaptic glycoprotein SC2 |
| | | | | | | | [Caenorhabditis elegans] |
| 2952 | LIB3602-117-Q1-K1-B12 | 70640 | BLASTX | 223 | 1.00E−18 | 100 | ubiquitin precursor - Trypanosoma cruzi (fragment) |
| | | | | | | | >gi\|10674\|emb\|CAA30334.1\| (X07451) ubiquitin |
| | | | | | | | fusion protein (78 AA) [Trypanosoma cruzi] |
| 2953 | LIB3602-120-Q1-K1-G10 | 1362064 | BLASTX | 369 | 6.00E−52 | 79 | GTP-binding protein - garden pea |
| | | | | | | | >gi\|871514\|emb\|CAA90079.1\| (Z49899) small G |
| | | | | | | | protein [Pisum sativum] |
| 2954 | LIB3602-095-Q6-K6-E12 | 6094211 | BLASTX | 248 | 7.00E−21 | 73 | PROBABLE SMALL NUCLEAR |
| | | | | | | | RIBONUCLEOPROTEIN G (SNRNP-G) (SM |
| | | | | | | | PROTEIN G) >gi\|3738322\|gb\|AAC63663.1\| |
| | | | | | | | (AC005170) putative small nuclear |
| | | | | | | | ribonucleoprotein G [Arabidopsis thaliana] |
| 2955 | LIB3602-112-Q1-K1-E10 | 3122684 | BLASTX | 368 | 7.00E−35 | 61 | 50S RIBOSOMAL PROTEIN L18 |
| | | | | | | | >gi\|2446905\|dbj\|BAA22464.1\| (AB000111) 50S |
| | | | | | | | ribosomal protein L18 [Synechococcus sp.] |
| 2956 | LIB3602-028-Q6-K1-B2 | 4544445 | BLASTX | 154 | 6.00E−10 | 57 | (AC006592) putative pyrophosphate--fructose-6- |
| | | | | | | | phosphate 1-phosphotransferase [Arabidopsis |
| | | | | | | | thaliana] |
| 2957 | LIB3602-067-Q1-K1-C1 | 5803265 | BLASTX | 227 | 2.00E−18 | 48 | (AP000399) similar to protein arginine N-methyl |
| | | | | | | | transferase 1 (Q63009) [Oryza sativa] |
| 2958 | LIB3602-047-Q6-K1-F6 | 3915072 | BLASTX | 266 | 4.00E−23 | 68 | PROBABLE METHIONYL-TRNA |
| | | | | | | | SYNTHETASE (METHIONINE--TRNA LIGASE) |
| | | | | | | | (METRS) >gi\|7504593\|pir\|\|T22898 hypothetical |
| | | | | | | | protein F58B3.5 - Caenorhabditis elegans |
| | | | | | | | >gi\|3877884\|emb\|CAA97803.1\| (Z73427) Similarity |
| | | | | | | | to Yeast methionyl-tRNA synthetase |
| | | | | | | | (SW: SYMC_YEAST); cDNA EST |
| 2959 | LIB3602-028-Q6-K1-A3 | 2109293 | BLASTX | 322 | 1.00E−29 | 58 | (U97568) serine/threonine protein kinase |
| | | | | | | | [Arabidopsis thaliana] |
| 2960 | LIB3602-065-Q1-K6-D3 | 7488039 | BLASTX | 325 | 4.00E−30 | 55 | probable ABC-type transport protein T23J7.100 - |
| | | | | | | | Arabidopsis thaliana |
| | | | | | | | >gi\|4741194\|emb\|CAB41860.1\| (AL049746) ABC |
| | | | | | | | transporter-like protein [Arabidopsis thaliana] |
| 2961 | LIB3602-080-Q6-K6-F3 | 120715 | BLASTX | 341 | 9.00E−32 | 72 | GLYCERALDEHYDE 3-PHOSPHATE |
| | | | | | | | DEHYDROGENASE (GAPDH) |
| | | | | | | | >gi\|66006\|pir\|\|DEKZGR glyceraldehyde-3- |
| | | | | | | | phosphate dehydrogenase (EC 1.2.1.12) - yeast |
| | | | | | | | (Zygosaccharomyces rouxii) |
| | | | | | | | >gi\|218529\|dbj\|BAA00081.1\| (D00134) |
| | | | | | | | glyceraldehyde-3-phosphate dehydrogenase |
| | | | | | | | [Zygosaccharomyce |
| 2962 | LIB3602-094-Q6-K6-C6 | 3913467 | BLASTX | 158 | 2.00E−10 | 57 | DFRA PROTEIN >gi\|2736191\|gb\|AAB97678.1\| |
| | | | | | | | (AF025847) DfrA [Myxococcus xanthus] |
| 2963 | LIB3602-009-Q6-K1-C3 | 2494266 | BLASTX | 513 | 5.00E−52 | 68 | GTP-BINDING PROTEIN LEPA |
| | | | | | | | >gi\|7443608\|pir\|\|S76959 GTP-binding membrane |
| | | | | | | | protein lepA - Synechocystis sp. (strain PCC 6803) |
| | | | | | | | >gi\|1653961\|dbj\|BAA18871.1\| (D90917) LepA |
| | | | | | | | [Synechocystis sp.] |
| 2964 | LIB3602-037-Q6-K1-H7 | 4966352 | BLASTX | 273 | 6.00E−24 | 42 | (AC006341) >F3O9.11 [Arabidopsis thaliana] |
| 2965 | LIB3602-015-Q6-K1-B10 | 7495172 | BLASTX | 250 | 2.00E−21 | 47 | hypothetical protein C02B10.1 - Caenorhabditis |
| | | | | | | | elegans >gi\|2702376\|gb\|AAB92016.1\| (AF038605) |
| | | | | | | | Similar to acyl-CoA dehydrogenase; coded for by |
| | | | | | | | C. elegans cDNA yk58h2.3; coded for by C. elegans |
| | | | | | | | cDNA yk466c12.3; coded for by C. elegans cDNA |
| | | | | | | | yk258d6.3; coded f |
| 2966 | LIB3602-007-Q1-K1-E1 | 7493715 | BLASTX | 362 | 4.00E−34 | 36 | WD repeat protein - fission yeast |
| | | | | | | | (Schizosaccharomyces pombe) |
| | | | | | | | >gi\|5701965\|emb\|CAB52157.1\| (AL109736) WD |
| | | | | | | | repeat protein [Schizosaccharomyces pombe] |
| 2967 | LIB3602-023-Q6-K1-D11 | 474005 | BLASTN | 37 | 5.00E−11 | 89 | Rice mRNA, partial homologous to ribosomal |
| | | | | | | | protein S5 gene |
| 2968 | LIB3602-041-Q6-K1-G7 | 7019653 | BLASTX | 471 | 4.00E−47 | 57 | (AL132954) spliceosomal-like protein [Arabidopsis |
| | | | | | | | thaliana] >gi\|7019655\|emb\|CAB75756.1\| |
| | | | | | | | (AL132954) spliceosomal-like protein [Arabidopsis |
| | | | | | | | thaliana] |
| 2969 | LIB3602-090-Q6-K6-G1 | 974301 | BLASTN | 67 | 6.00E−29 | 91 | Chlamydomonas reinhardtii ribosomal protein L41 |
| | | | | | | | (RPL41) mRNA, complete cds |
| 2970 | LIB3602-087-Q6-K1-F12 | 7486098 | BLASTX | 189 | 7.00E−14 | 49 | hypothetical protein F24L7.22 - Arabidopsis |
| | | | | | | | thaliana >gi\|2914709\|gb\|AAC04499.1\| (AC003974) |
| | | | | | | | hypothetical protein [Arabidopsis thaliana] |
| 2971 | LIB3602-078-Q6-K6-H8 | 849081 | BLASTX | 565 | 5.00E−58 | 63 | (U17900) cyclophilin B precursor [Orpinomyces sp. |
| | | | | | | | PC-2] |
| 2972 | LIB3602-014-Q6-K1-B4 | 1346031 | BLASTX | 536 | 1.00E−54 | 57 | FARNESYL PYROPHOSPHATE SYNTHETASE |
| | | | | | | | (FPP SYNTHETASE) (FPS) (FARNESYL |
| | | | | | | | DIPHOSPHATE SYNTHETASE) (KIAA0032) |
| | | | | | | | [INCLUDES: |
| | | | | | | | DIMETHYLALLYLTRANSFERASE; |
| | | | | | | | GERANYLTRANSTRANSFERASE] |
| 2973 | LIB3602-104-Q1-K1-C1 | 6094228 | BLASTX | 371 | 2.00E−35 | 71 | ADENOSYLHOMOCYSTEINASE (S- |
| | | | | | | | ADENOSYL-L-HOMOCYSTEINE |
| | | | | | | | HYDROLASE) (ADOHCYASE) |
| | | | | | | | >gi\|1724102\|gb\|AAB38499.1\| (U79766) S- |
| | | | | | | | adenosyl-L-homocystein hydrolase; SAH |
| | | | | | | | [Mesembryanthemum crystallinum] |
| 2974 | LIB3602-058-Q6-K1-F12 | 5803275 | BLASTX | 179 | 7.00E−13 | 42 | (AP000399) EST AU056133(S20320) corresponds |
| | | | | | | | to a region of the predicted gene; similar to |
| | | | | | | | Caenorhabditis elegans cosmid D1054; hypothetical |
| | | | | | | | protein (Z74030) [Oryza sativa] |
| 2975 | LIB3602-005-Q1-K1-G11 | 3834326 | BLASTX | 147 | 6.00E−09 | 53 | (AC005679) Similar to gb\|AF067141 gamma- |
| | | | | | | | glutamyl hydrolase from Arabidopsis thaliana. ESTs |
| | | | | | | | gb\|T46595 and gb\|AI09918 come from this gene |
| 2976 | LIB3602-013-Q6-K1-G4 | 7431450 | BLASTX | 359 | 5.00E−34 | 61 | aldehyde dehydrogenase (NAD+) (EC 1.2.1.3) |
| | | | | | | | T17F15.130 - Arabidopsis thaliana |
| | | | | | | | >gi\|4678328\|emb\|CAB41139.1\| (AL049658) |
| | | | | | | | aldehyde dehydrogenase (NAD+)-like protein |
| | | | | | | | [Arabidopsis thaliana] |
| 2977 | LIB3602-021-Q6-K1-F7 | 1172809 | BLASTX | 355 | 7.00E−34 | 72 | 60S RIBOSOMAL PROTEIN L10 (QM PROTEIN |
| | | | | | | | HOMOLOG) >gi\|7447294\|pir\|\|T02068 probable |
| | | | | | | | transcription factor QM - maize |
| | | | | | | | >gi\|468056\|gb\|AAA17419.1\| (U06108) QM protein |
| | | | | | | | [Zea mays] |
| 2978 | LIB3602-052-Q6-K1-H2 | 3334230 | BLASTX | 342 | 5.00E−32 | 47 | D-HYDANTOINASE |
| | | | | | | | (DIHYDROPYRIMIDINASE) (DHPASE) |
| | | | | | | | >gi\|2828803\|gb\|AAC00209.1\| (U84197) D- |
| | | | | | | | hydantoinase [Pseudomonas putida] |
| 2979 | LIB3602-014-Q6-K1-B7 | 2811057 | BLASTX | 164 | 4.00E−11 | 34 | CHITOSANASE PRECURSOR |
| | | | | | | | >gi\|7474500\|pir\|\|C69608 chitosanase csn - Bacillus |
| | | | | | | | subtilis >gi\|1934630\|gb\|AAB80882.1\| (U93875) |
| | | | | | | | chitosanase precursor [Bacillus subtilis] |
| | | | | | | | >gi\|2108281\|emb\|CAA63455.1\| (X92868) |
| | | | | | | | chitosanase precursor [Bacillus subtilis] |
| | | | | | | | >gi\|2635134\|emb\| |
| 2980 | LIB3602-093-Q6-K6-E2 | 3738312 | BLASTX | 270 | 2.00E−23 | 38 | (AC005309) hypothetical protein [Arabidopsis |
| | | | | | | | thaliana] |
| 2981 | LIB3602-023-Q6-K1-D6 | 1172835 | BLASTX | 338 | 2.00E−31 | 75 | GTP-BINDING NUCLEAR PROTEIN RAN-A1 |
| | | | | | | | >gi\|496268\|gb\|AAA73563.1\| (L16767) GTP- |
| | | | | | | | binding protein [Nicotiana tabacum] |
| 2982 | LIB3602-078-Q6-K6-F11 | 7435073 | BLASTX | 209 | 2.00E−16 | 41 | hypothetical protein T26B15.8 - Arabidopsis |
| | | | | | | | thaliana >gi\|3298540\|gb\|AAC25934.1\| (AC004681) |
| | | | | | | | putative carboxymethylenebutenolidase |
| | | | | | | | [Arabidopsis thaliana] |
| 2983 | LIB3602-080-Q6-K6-D6 | 5902716 | BLASTX | 174 | 1.00E−12 | 47 | ACYL-COA-BINDING PROTEIN HOMOLOG |
| | | | | | | | (ACBP) (DIAZEPAM BINDING INHIBITOR |
| | | | | | | | HOMOLOG) (DBI) >gi\|7497423\|pir\|\|T30954 |
| | | | | | | | hypothetical protein C44E4.6 - Caenorhabditis |
| | | | | | | | elegans >gi\|2088729\|gb\|AAB54171.1\| (AF003140) |
| | | | | | | | Similar to acyl-CoA-binding protein; C44E4.6 |
| | | | | | | | [Caenorhabd |
| 2984 | LIB3602-016-Q6-K1-B5 | 1084468 | BLASTX | 147 | 4.00E−09 | 33 | hydroxymandelonitrile lyase (EC 4.1.2.11) chain A - |
| | | | | | | | sorghum (fragment) |
| 2985 | LIB3602-010-Q6-K1-A6 | 6899925 | BLASTX | 380 | 2.00E−36 | 70 | (AL138651) P-glycoprotein-like proetin |
| | | | | | | | [Arabidopsis thaliana] |
| 2986 | LIB3602-032-Q6-K1-F11 | 7488412 | BLASTX | 229 | 1.00E−18 | 49 | ubiquitin-specific proteinase (EC 3.4.—.—) UBP3 - |
| | | | | | | | Arabidopsis thaliana >gi\|2347098\|gb\|AAB67966.1\| |
| | | | | | | | (U76845) ubiquitin-specific protease [Arabidopsis |
| | | | | | | | thaliana] >gi\|4490742\|emb\|CAB38904.1\| |
| | | | | | | | (AL035708) ubiquitin-specific protease (AtUBP3) |
| | | | | | | | [Arabidopsis thaliana |
| 2987 | LIB3602-102-Q1-K1-E12 | 3482918 | BLASTX | 305 | 1.00E−27 | 71 | (AC003970) Similar to ATP-citrate-lyase |
| | | | | | | | [Arabidopsis thaliana] |
| 2988 | LIB3602-009-Q6-K1-E1 | 7379550 | BLASTX | 160 | 1.00E−10 | 40 | (AL162754) hypothetical protein NMA0833 |
| | | | | | | | [Neisseria meningitidis] |
| 2989 | LIB3602-003-Q1-K1-A5 | 7269961 | BLASTX | 211 | 5.00E−17 | 73 | (AL161577) signal recognition particle receptor-like |
| | | | | | | | protein [Arabidopsis thaliana] |
| 2990 | LIB3602-092-Q6-K6-B1 | 7439981 | BLASTX | 146 | 9.00E−14 | 68 | glycine-rich RNA-binding protein GRP1 wheat |
| | | | | | | | >gi\|974605\|gb\|AAA75104.1\| (U32310) single- |
| | | | | | | | stranded nucleic acid binding protein [Triticum |
| | | | | | | | aestivum] |
| 2991 | LIB3602-026-Q6-K1-G11 | 480616 | BLASTX | 689 | 1.00E−72 | 80 | chlorophyll a/b binding protein - prince's feather |
| | | | | | | | >gi\|398599\|emb\|CAA52750.1\| (X74732) |
| | | | | | | | chlorophyll a/b binding protein [Amaranthus |
| | | | | | | | hypochondriacus] |
| 2992 | LIB3602-092-Q6-K6-G2 | 7486479 | BLASTX | 514 | 6.00E−52 | 73 | hypothetical protein F6E13.19 - Arabidopsis |
| | | | | | | | thaliana >gi\|3212877\|gb\|AAC23428.1\| (AC004005) |
| | | | | | | | putative LEA (late embryogenesis abundant) protein |
| | | | | | | | [Arabidopsis thaliana] |
| 2993 | LIB3602-055-Q6-K1-H10 | 5902394 | BLASTX | 220 | 1.00E−17 | 33 | (AC008148) Putative phosphoglucomutase |
| | | | | | | | [Arabidopsis thaliana] |
| 2994 | LIB3602-093-Q6-K6-G6 | 4574242 | BLASTX | 463 | 2.00E−48 | 78 | (AF108725) ribosomal protein S16 [Tortula ruralis] |
| 2995 | LIB3602-025-Q6-K1-F7 | 6714424 | BLASTX | 554 | 9.00E−57 | 81 | (AC012328) putative ADP-ribosylation factor |
| | | | | | | | [Arabidopsis thaliana] |
| 2996 | LIB3602-025-Q6-K1-B3 | 2129619 | BLASTX | 325 | 6.00E−30 | 73 | ribosome-associated protein p40 homolog - |
| | | | | | | | Arabidopsis thaliana |
| | | | | | | | >gi\|1345503\|emb\|CAA61547.1\| (X89366) 40 kD |
| | | | | | | | protein [Arabidopsis thaliana] |
| | | | | | | | >gi\|2102657\|emb\|CAA71407.1\| (Y10379) unnamed |
| | | | | | | | protein product [Arabidopsis thaliana] |
| 2997 | LIB3602-034-Q6-K1-H11 | 3738327 | BLASTX | 220 | 9.00E−18 | 39 | (AC005170) putative serine carboxypeptidase II |
| | | | | | | | [Arabidopsis thaliana] |
| 2998 | LIB3602-035-Q1-K1-B9 | 3738328 | BLASTX | 397 | 3.00E−38 | 54 | (AC005170) putative serine carboxypeptidase II |
| | | | | | | | [Arabidopsis thaliana] |
| 2999 | LIB3602-022-Q6-K1-F6 | 7489162 | BLASTX | 510 | 1.00E−51 | 68 | kinesin-related protein tck1 - common tobacco |
| | | | | | | | >gi\|1491931\|gb\|AAC49393.1\| (U52078) kinesin- |
| | | | | | | | like protein [Nicotiana tabacum] |
| 3000 | LIB3602-026-Q6-K1-G7 | 7488433 | BLASTX | 245 | 2.00E−20 | 42 | zinc finger protein homolog T10M13.22 - |
| | | | | | | | Arabidopsis thaliana >gi\|3377805\|gb\|AAC28178.1\| |
| | | | | | | | (AF075597) contains similarity to several apoptosis |
| | | | | | | | or programmed cell death proteins such as rat |
| | | | | | | | apoptosis protein RP-8 (GB: M80601) [Arabidopsis |
| | | | | | | | thaliana] >gi\|3912928 |
| 3001 | LIB3602-025-Q6-K1-D9 | 6554202 | BLASTX | 150 | 2.00E−09 | 43 | (AC011661) T23J18.5 [Arabidopsis thaliana] |
| 3002 | LIB3602-082-Q6-K6-E9 | 1170898 | BLASTX | 376 | 1.00E−49 | 77 | MALATE DEHYDROGENASE, |
| | | | | | | | MITOCHONDRIAL PRECURSOR |
| | | | | | | | >gi\|629659\|pir\|\|S44167 malate dehydrogenase (EC |
| | | | | | | | 1.1.1.37), mitochondrial - cider tree |
| | | | | | | | >gi\|473206\|emb\|CAA55383.1\| (X78800) |
| | | | | | | | mitochondrial malate dehydrogenase [Eucalyptus |
| | | | | | | | gunnii] |
| 3003 | LIB3602-052-Q6-K1-B6 | 7190377 | BLASTX | 270 | 1.00E−23 | 38 | (AE002301) ADP, ATP carrier protein [Chlamydia |
| | | | | | | | muridarum] |
| 3004 | LIB3602-062-Q6-K1-C1 | 7297593 | BLASTX | 147 | 4.00E−09 | 32 | (AE003627) CG4747 gene product [Drosophila |
| | | | | | | | melanogaster] |
| 3005 | LIB3602-119-Q1-K1-E11 | 7439992 | BLASTX | 168 | 6.00E−12 | 50 | splicing factor RSZp22 - Arabidopsis thaliana |
| | | | | | | | >gi\|3281869\|emb\|CAA19765.1\| (AL031004) |
| | | | | | | | RSZp22 splicing factor [Arabidopsis thaliana] |
| | | | | | | | >gi\|3435094\|gb\|AAD12769.1\| (AF033586) 9G8- |
| | | | | | | | like SR protein [Arabidopsis thaliana] |
| | | | | | | | >gi\|7270061\|emb\|CAB79876.1\| (AL161579) RSZp |
| 3006 | LIB3602-042-Q6-K1-H2 | 7470311 | BLASTX | 200 | 2.00E−15 | 49 | hypothetical protein slr0919 - Synechocystis sp. |
| | | | | | | | (strain PCC 6803) >gi\|1001227\|dbj\|BAA10467.1\| |
| | | | | | | | (D64003) hypothetical protein [Synechocystis sp.] |
| 3007 | LIB3602-054-Q6-K1-C8 | 3334244 | BLASTX | 378 | 3.00E−36 | 65 | LACTOYLGLUTATHIONE LYASE |
| | | | | | | | (METHYLGLYOXALASE) |
| | | | | | | | (ALDOKETOMUTASE) (GLYOXALASE I) (GLXI) |
| | | | | | | | (KETONE-ALDEHYDE MUTASE) (S-D- |
| | | | | | | | LACTOYLGLUTATHIONE METHYLGLYOXAL |
| | | | | | | | LYASE) >gi\|2113825\|emb\|CAA73691.1\| (Y13239) |
| | | | | | | | Glyoxalase I [Brassica juncea] |
| 3008 | LIB3602-086-Q6-K1-D9 | 6899939 | BLASTX | 269 | 1.00E−23 | 81 | (AL138651) ADP-ribosylation factor-like protein |
| | | | | | | | [Arabidopsis thaliana] |
| 3009 | LIB3602-092-Q6-K6-B5 | 498741 | BLASTN | 325 | 0 | 93 | H. vulgare (pMaW25) mRNA for beta-ketoacyl- |
| | | | | | | | ACP synthase |
| 3010 | LIB3602-048-Q6-K1QA- | 7430935 | BLASTX | 566 | 3.00E−58 | 63 | probable cinnamyl-alcohol dehydrogenase (EC |
| | E10 | | | | | | 1.1.1.195) - tomato |
| 3011 | LIB3602-120-Q1-K1-E6 | 567890 | BLASTX | 187 | 1.00E−13 | 46 | (L37352) beta-galactosidase-complementation |
| | | | | | | | protein [unidentified cloning vector] |
| 3012 | LIB3602-013-Q6-K1-B3 | 228698 | BLASTX | 584 | 3.00E−60 | 58 | CF1 ATP synthase: SUBUNIT = gamma |
| | | | | | | | [Chlamydomonas reinhardtii] |
| 3013 | LIB3602-107-Q1-K1-F10 | 1711381 | BLASTX | 295 | 2.00E−26 | 65 | PHOSPHOSERINE AMINOTRANSFERASE |
| | | | | | | | PRECURSOR (PSAT) >gi\|7434195\|pir\|\|T09156 |
| | | | | | | | phosphoserine aminotransferase - spinach |
| | | | | | | | >gi\|1256204\|dbj\|BAA12206.1\| (D84061) |
| | | | | | | | phosphoserine aminotransferase [Spinacia oleracea] |
| 3014 | LIB3602-119-Q1-K1-E9 | 585876 | BLASTX | 197 | 2.00E−15 | 63 | 60S RIBOSOMAL PROTEIN L23A (L25) |
| | | | | | | | >gi\|1084424\|pir\|\|S48026 ribosomal protein L23a, |
| | | | | | | | cytosolic - common tobacco |
| | | | | | | | >gi\|310935\|gb\|AAA53296.1\| (L18908) 60S |
| | | | | | | | ribosomal protein L25 [Nicotiana tabacum] |
| 3015 | LIB3602-023-Q6-K1-A1 | 1351905 | BLASTX | 203 | 1.00E−15 | 46 | BIFUNCTIONAL |
| | | | | | | | ASPARTOKINASE/HOMOSERINE |
| | | | | | | | DEHYDROGENASE 2, CHLOROPLAST |
| | | | | | | | PRECURSOR (AK-HD 2) (AK-HSDH 2) |
| | | | | | | | [INCLUDES: ASPARTOKINASE; |
| | | | | | | | HOMOSERINE DEHYDROGENASE] |
| | | | | | | | >gi\|7434567\|pir\|\|T02954 aspartate kinase (EC |
| | | | | | | | 2.7.2.4)/homoserine dehydrogenase (EC 1.1.1.3) |
| | | | | | | | precurso |
| 3016 | LIB3602-038-Q6-K1-C6 | 3851612 | BLASTX | 616 | 4.00E−64 | 67 | (AF095937) succinate dehydrogenase Ip subunit |
| | | | | | | | [Gallus gallus] |
| 3017 | LIB3602-111-Q1-K1-D4 | 7450363 | BLASTX | 477 | 5.00E−48 | 76 | hypothetical protein T9E8.170 - Arabidopsis |
| | | | | | | | thaliana >gi\|4584548\|emb\|CAB40778.1\| |
| | | | | | | | (AL049608) putative protein [Arabidopsis thaliana] |
| | | | | | | | >gi\|7268046\|emb\|CAB78385.1\| (AL161536) |
| | | | | | | | putative protein [Arabidopsis thaliana] |
| 3018 | LIB3602-017-Q6-K1-G8 | 7484678 | BLASTX | 524 | 3.00E−53 | 59 | phosphoglucomutase precursor, chloroplast - |
| | | | | | | | spinach >gi\|534982\|emb\|CAA53507.1\| (X75898) |
| | | | | | | | phosphoglucomutase [Spinacia oleracea] |
| 3019 | LIB3602-118-Q1-K1-G5 | 4646217 | BLASTX | 778 | 5.00E−83 | 76 | (AC007290) putative phosphoprotein phosphatase |
| | | | | | | | [Arabidopsis thaliana] |
| 3020 | LIB3602-037-Q6-K1-F2 | 400515 | BLASTX | 225 | 3.00E−18 | 54 | NADH-UBIQUINONE OXIDOREDUCTASE B8 |
| | | | | | | | SUBUNIT (COMPLEX I-B8) (CI-B8) |
| | | | | | | | >gi\|346540\|pir\|\|S28249 NADH dehydrogenase |
| | | | | | | | (ubiquinone) (EC 1.6.5.3) chain CI-B8 - bovine |
| | | | | | | | >gi\|246\|emb\|CAA44904.1\| (X63219) NADH |
| | | | | | | | dehydrogenase [Bos taurus] |
| 3021 | LIB3602-048-Q6-K1QA-B8 | 7019681 | BLASTX | 573 | 5.00E−59 | 75 | (AL 138647) putative protein [Arabidopsis thaliana] |
| 3022 | LIB3602-076-Q6-K6-G9 | 6440857 | BLASTX | 678 | 3.00E−71 | 77 | (AU066528) 40S ribosomal protein S9 |
| | | | | | | | [Chlamydomonas sp.] |
| 3023 | LIB3602-092-Q6-K6-H7 | 904068 | BLASTN | 66 | 3.00E−28 | 92 | Hordeum vulgare (clone ABC167) STS mRNA |
| 3024 | LIB3602-051-Q6-K1-G1 | 2494299 | BLASTX | 411 | 3.00E−40 | 68 | EUKARYOTIC TRANSLATION INITIATION |
| | | | | | | | FACTOR 2 BETA SUBUNIT (EIF-2-BETA) |
| | | | | | | | >gi\|7522212\|pir\|\|T17104 translation initiation factor |
| | | | | | | | eIF-2 beta chain - apple tree (fragment) |
| | | | | | | | >gi\|1732361\|gb\|AAC06384.1\| (U80269) translation |
| | | | | | | | initiation factor 2 beta [Malus domestica] |
| 3025 | LIB3602-017-Q6-K1-D11 | 7492850 | BLASTX | 163 | 5.00E−11 | 62 | probable prohibitin antiproliferative protein - fission |
| | | | | | | | yeast (Schizosaccharomyces pombe) |
| | | | | | | | >gi\|4176556\|emb\|CAA22869.1\| (AL035259) |
| | | | | | | | putative prohibitin [Schizosaccharomyces pombe] |
| 3026 | LIB3602-049-Q6-K1-F5 | 7414558 | BLASTX | 265 | 5.00E−23 | 55 | (AL163003) putative fatty acid synthase |
| | | | | | | | [Streptomyces coelicolor A3(2)] |
| 3027 | LIB3602-004-Q1-K1-B2 | 1172494 | BLASTX | 150 | 3.00E−09 | 50 | PTERIN-4-ALPHA-CARBINOLAMINE |
| | | | | | | | DEHYDRATASE (PHS) (4-ALPHA-HYDROXY- |
| | | | | | | | TETRAHYDROPTERIN DEHYDRATASE) |
| | | | | | | | (PHENYLALANINE HYDROXYLASE- |
| | | | | | | | STIMULATING PROTEIN) (PCD) |
| | | | | | | | >gi\|476742\|gb\|AAA25937.1\| (M88627) |
| | | | | | | | phenylalanine hydroxylase [Pseudomonas |
| | | | | | | | aeruginosa] |
| 3028 | LIB3602-003-Q1-K1-E2 | 6939228 | BLASTX | 452 | 6.00E−45 | 61 | (AC020579) putative |
| | | | | | | | phosphoribosylformylglycinamidine synthase |
| | | | | | | | [Arabidopsis thaliana] |
| 3029 | LIB3602-028-Q6-K1-D4 | 7487199 | BLASTX | 265 | 7.00E−23 | 42 | hypothetical protein T19L18.24 - Arabidopsis |
| | | | | | | | thaliana >gi\|3413717\|gb\|AAC31240.1\| (AC004747) |
| | | | | | | | unknown protein [Arabidopsis thaliana] |
| 3030 | LIB3602-113-Q1-K1-B1 | 7439979 | BLASTX | 371 | 2.00E−35 | 97 | glycine-rich RNA-binding protein, low- |
| | | | | | | | temperature-responsive - barley |
| | | | | | | | >gi\|1229138\|gb\|AAB07749.1\| (U49482) low |
| | | | | | | | temperature-responsive RNA-binding protein |
| | | | | | | | [Hordeum vulgare] |
| 3031 | LIB3602-080-Q6-K6-A7 | 4502327 | BLASTX | 453 | 7.00E−45 | 49 | AU RNA-binding protein/enoyl-Coenzyme A |
| | | | | | | | hydratase precursor >gi\|2135070\|pir\|\|I37195 AU- |
| | | | | | | | specific RNA-binding protein/enoyl-CoA |
| | | | | | | | hydratase (EC 4.2.1.17) homolog - human |
| | | | | | | | >gi\|780241\|emb\|CAA56260.1\| (X79888) AU- |
| | | | | | | | binding protein/Enoyl-CoA hydratase [Homo sapien |
| 3032 | LIB3602-102-Q1-K1-E6 | 6822051 | BLASTX | 328 | 9.00E−46 | 64 | (AL132957) fructose-bisphosphatase precursor |
| | | | | | | | [Arabidopsis thaliana] |
| 3033 | LIB3602-083-Q6-K6-C2 | 3063365 | BLASTX | 452 | 1.00E−44 | 49 | (AB003712) elongation factor-1alpha |
| | | | | | | | [Ampharetidae sp.] |
| 3034 | LIB3602-111-Q1-K1-G12 | 4580461 | BLASTX | 343 | 4.00E−32 | 44 | (AC006081) unknown protein [Arabidopsis |
| | | | | | | | thaliana] |
| 3035 | LIB3602-039-Q6-K1-C5 | 7487355 | BLASTX | 186 | 1.00E−13 | 57 | hypothetical protein T22J18.13 - Arabidopsis |
| | | | | | | | thaliana >gi\|3287688\|gb\|AAC25516.1\| (AC003979) |
| | | | | | | | Contains similarity to ycf37 gene product |
| | | | | | | | gb\|1001425 from Synechocystis sp. genome |
| | | | | | | | gb\|D63999. ESTs gb\|T43026, gb\|R64902, |
| | | | | | | | gb\|Z18169 and gb\|N37374 come from this ge |
| 3036 | LIB3602-060-Q6-K1-C3 | 6513923 | BLASTX | 490 | 3.00E−49 | 67 | (AC011664) putative mitochondrial processing |
| | | | | | | | peptidase [Arabidopsis thaliana] |
| 3037 | LIB3602-091-Q6-K6-A1 | 7503664 | BLASTX | 168 | 2.00E−11 | 32 | hypothetical protein F47C12.1 - Caenorhabditis |
| | | | | | | | elegans >gi\|1397258\|gb\|AAC24388.1\| (U61946) |
| | | | | | | | contains multiple regions of similarity to EGF-type |
| | | | | | | | repeats (PS: PS00022) and sushi (SCR) repeats |
| | | | | | | | [Caenorhabditis elegans] |
| 3038 | LIB3602-077-Q6-K6-F4 | 7019692 | BLASTX | 479 | 7.00E−48 | 61 | (AL 138647) putative protein [Arabidopsis thaliana] |
| 3039 | LIB3602-004-Q1-K1-E6 | 3914557 | BLASTX | 231 | 4.00E−36 | 76 | RAS-RELATED PROTEIN RAB7 (POSSIBLE |
| | | | | | | | APOSPORY-ASSOCIATED PROTEIN) |
| | | | | | | | >gi\|1155265\|gb\|AAA85273.1\| (U40219) possible |
| | | | | | | | apospory-associated protein [Pennisetum ciliare] |
| 3040 | LIB3602-004-Q1-K1-D8 | 6862923 | BLASTX | 258 | 6.00E−22 | 56 | (AC018907) unknown protein [Arabidopsis |
| | | | | | | | thaliana] |
| 3041 | LIB3602-009-Q6-K1-F6 | 2828267 | BLASTX | 262 | 3.00E−23 | 75 | (Y14044) geranylgeranyl reductase [Arabidopsis |
| | | | | | | | thaliana] |
| 3042 | LIB3602-102-Q1-K1-A1 | 7019693 | BLASTX | 304 | 7.00E−28 | 64 | (AL138647) putative protein [Arabidopsis thaliana] |
| 3043 | LIB3602-016-Q6-K1-H4 | 6862921 | BLASTX | 170 | 9.00E−12 | 54 | (AC018907) hypothetical protein [Arabidopsis |
| | | | | | | | thaliana] |

Table 1 Legend
SEQ ID No.: Refers to the sequence identification number of nucleic acids listed in the Sequence Listing.
Clone ID: Refers to an assigned cDNA clone ID number.
NCBI gi: Refers to National Center for Biotechnology Information GenBank Identifier number which is the best match for a given nucleotide sequence.
Method: Refers to the method used in the sequence comparison of the designated nucleotide sequence with the designated GenBank sequence.
BLAST Score: Refers to the BLAST score that is generated by sequence comparison of the designated nucleotide sequence with the designated GenBank sequence using the method referenced in the “Method” column.
E value: The expected number of distinct segment pairs between two sequences with a score above the bit score for BLAST match.
% ident: Refers to the percentage of identically matched nucleotides (or residues) that exist along the length of that portion of the sequences which is aligned by the BLAST comparison to generate the statistical scores presented.
NCBI gi Description: A description of the database entry referenced in the “NCBI gi” column.

1. An isolated nucleic acid molecule comprising a nucleotide sequence of SEQ ID NO: 2 or complement thereof.

2. The isolated nucleic acid molecule of claim 1, wherein said isolated nucleic acid molecule encodes a Chlorella sarokiniana protein.

3. The isolated nucleic acid molecule of claim 2, wherein said Chlorella sarokiniana protein is a homologue of a 60S Ribosomal protein.

4. The isolated nucleic acid molecule of claim 3, wherein said isolated nucleic acid molecule a nucleotide sequence of SEQ ID: 2.

5. (canceled)

6. A transformed cell having an exogenous nucleic acid molecule which comprises:
(A) an exogenous promoter region which functions in said cell to cause the production of a mRNA molecule; which is operably linked to
(B) a structural nucleic acid molecule, wherein said structural nucleic acid molecule comprises a nucleic acid sequence of SEQ ID NO: 2 or complement thereof; which is operably linked to
(C) a 3′ non-translated sequence that functions in said cell to cause termination of transcription and addition of polyadenylated ribonucleotides to a 3′ end of said mRNA molecule.

7. The transformed cell according to claim 6, wherein said cell is selected from the group consisting of an algal cell, a plant cell, a mammalian cell, a bacterial cell, a fungal cell and an insect cell.

8. The transformed cell according to claim 7, wherein said cell is an algal cell.

9. The transformed cell according to claim 8, wherein said cell is a Chlorella sarokiniana cell.

10. The transformed cell according to claim 7, wherein said cell is a plant cell.

11. A substantially purified nucleic acid molecule consisting of a nucleic acid sequence of SEQ ID NO: 2 or complement thereof.

12. A substantially purified nucleic acid molecule comprising a nucleic acid sequence having between 100% and 90% sequence identity with a nucleic acid sequence of SEQ ID NO: 2 or complement thereof.

13. The substantially purified nucleic acid molecule of claim 12, wherein said nucleic acid molecule comprises a nucleic acid sequence having between 100% and 95% sequence identity with a nucleic acid sequence of SEQ ID NO: 2 or complement thereof.

14. The substantially purified nucleic acid molecule of claim 13, wherein said nucleic acid molecule comprises a nucleic acid sequence having between 100% and 98% sequence identity with a nucleic acid sequence of SEQ ID NO: 2 or complement thereof.

15. The substantially purified nucleic acid molecule of claim 14, wherein said nucleic acid molecule comprises a nucleic acid sequence having between 100% and 99% sequence identity with a nucleic acid sequence of SEQ ID NO: 2 or complement thereof.
